(19) United States
(12) Patent Application Publication
(10) Pub. No.: US 2024/0299583 A1
(43) Pub. Date: Sep. 12, 2024

(54) Modified Guide RNAs for Gene Editing

(71) Applicant: Intellia Therapeutics, Inc., Cambridge, MA (US)

(72) Inventors: Sabin Mulepati, Somerville, MA (US); Lindsey Jean Stretz, Cambridge, MA (US); Michelle Young, Cambridge, MA (US); Sung Hee Choi, Cambridge, MA (US); Rubina Giare Parmar, Acton, MA (US); Weijun Chen, Shrewsbury, MA (US); Eun Soo Yoon, Cambridge, MA (US)

(73) Assignee: Intellia Therapeutics, Inc., Cambridge, MA (US)

(21) Appl. No.: 18/652,176

(22) Filed: May 1, 2024

Related U.S. Application Data
(63) Continuation of application No. PCT/US2022/079121, filed on Nov. 2, 2022.
(60) Provisional application No. 63/352,161, filed on Jun. 14, 2022.
(60) Provisional application No. 63/275,426, filed on Nov. 3, 2021.

(51) Int. Cl. A61K 48/00; C12N 9/22; C12N 9/78; C12N 15/11; C12N 15/88; C12N 15/90

(52) U.S. Cl. CPC A61K 48/005; C12N 9/22; C12N 9/78; C12N 15/11; C12N 15/88; C12N 15/907; C12Y 305/04005; C12N 2310/20; C12N 2310/531

(57) ABSTRACT

This disclosure relates to modified guide RNAs having improved in vitro and in vivo activity in gene editing methods. This disclosure also relates to N. meningitidis Cas9 (NmeCas9) gene editing systems with modified guide RNAs.

This application is a Continuation of International Application No. PCT/US2022/079121 filed on Nov. 2, 2022, which claims the benefit of priority to U.S. Provisional Application No. 63/275,426 filed on Nov. 3, 2021, and U.S. Provisional Application No. 63/352,161 filed on Jun. 14, 2022, the contents all of which are incorporated by reference in their entirety.

The instant application contains a Sequence Listing which has been submitted electronically in XML format and is hereby incorporated by reference in its entirety. Said XML copy, created on Nov. 1, 2022, is named 01155-0048-00PCT_ST26 and is 1,429,694 bytes in size.

This disclosure relates to the field of gene editing using CRISPR/Cas9 systems, a part of the prokaryotic immune system that recognizes and cuts exogenous genetic elements.

The CRISPR/Cas9 system relies on a single nuclease, termed CRISPR-associated protein 9 (Cas9), which induces site-specific breaks in DNA. Cas9 is guided to specific DNA sequences by small RNA molecules termed guide RNA (gRNA). A complete guide RNA comprises tracrRNA (trRNA) and crisprRNA (crRNA). A crRNA comprising a guide region may also be referred to as a gRNA, with the understanding that to form a complete gRNA it should be or become associated covalently or noncovalently with a trRNA. The trRNA and crRNA may be contained within a single guide RNA (sgRNA) or in two separate RNA molecules of a dual guide RNA (dgRNA). Cas9 in combination with gRNA is termed the Cas9 ribonucleoprotein complex (RNP).

CRISPR/Cas9 systems exist in various bacterial species, and can have different properties, including with respect to gRNA length and degree of sequence-specificity in cleavage. Neisseria meningitidis Cas9 (NmeCas9) has an advantageously low off-target cleavage rate but uses relatively long gRNAs, which complicates in vitro gRNA synthesis.

Oligonucleotides, and in particular RNA, are sometimes degraded in cells and in serum by non-enzymatic, endonuclease or exonuclease cleavage. Oligonucleotides can be synthesized with modifications at various positions to reduce or prevent such degradation. Given the cyclic nature and imperfect yield of oligonucleotide synthesis, shortening the gRNA while retaining or even improving its activity would be desirable, e.g., so that the gRNA can be obtained in greater yield, or compositions comprising the gRNA have greater homogeneity or fewer incomplete or erroneous products. Additionally, improved methods and compositions for preventing such degradation, improving stability of gRNAs and enhancing gene editing efficiency is desired, especially for therapeutic applications. The present disclosure aims to meet one or more of these needs, provide other benefits, or at least provide the public with a useful choice.

### SUMMARY

The present disclosure relates to gene editing using Neisseria meningitidis CRISPR/Cas9 systems. NmeCas9 is smaller than Streptococcus pyogenes Cas9 (SpyCas9), allowing NmeCas9 to be suitable for messenger RNA (mRNA)-based delivery methods. However, NmeCas9 forms an RNP with a gRNA that is longer than a SpyCas9 guide RNA. Conventionally used gRNA for NmeCas9 has a length of 145 or more nucleotides (Ibraheim et al. Genome Biology (2018) 19:137) and shortening the gRNA while retaining or even improving its activity would be desirable for preventing degradation and improving stability of gRNAs and enhancing gene editing efficiency.

In some embodiments, genome editing tools are provided comprising guide RNA (gRNA) with one or more shortened regions as described herein. The shortened regions described herein may facilitate synthesis of the gRNA with greater yield or homogeneity, or may improve the stability of the gRNA and the gRNA/Cas9 complex, or improve the activity of Cas9 to cleave target DNA.

In some embodiments, crisprRNA (crRNA) or tracrRNA (trRNA) with one or more shortened regions or substitutions as described herein are provided. In some embodiments, a dual guide RNA (dgRNA) comprises the modified crRNA or modified trRNA. In some embodiments, a single guide RNA (sgRNA) comprises the modified crRNA or modified trRNA. The shortened regions or substitutions described herein may facilitate synthesis of the gRNA with greater yield or homogeneity or may improve the stability of the gRNA and the gRNA/Cas9 complex, or improve the activity of NmeCas9 to cleave target DNA. Compared to NmeCas9 145-mer sgRNAs, synthesis of the presently disclosed gRNAs may increase crude yield of a gRNA. Similarly, gRNA sample purity as measured by the proportion of full length product, e.g., crude purity, can be increased. Guide RNA can be obtained in greater yield, or compositions comprising the gRNA can have greater homogeneity or fewer incomplete or erroneous products. Guide RNA purity may be assessed using ion-pair reversed-phase high performance liquid chromatography (IP-RP-HPLC) and ion exchange HPLC methods, e.g., as in Kanavarioti et al, Sci Rep 9, 1019 (2019) (doi:10.1038/s41598-018-37642-z). Using UV spectroscopy at a wavelength of 260 nm, crude purity and final purity can be determined by the ratio of absorbance of the main peak to the cumulative absorbance of all peaks in the chromatogram. Synthetic yield is determined as the ratio of the absorbance at 260 nm of the final sample compared to the theoretical absorbance of input materials.

The following embodiments are encompassed.

In some embodiments, a guide RNA (gRNA) is provided, the guide RNA comprising a guide region and a conserved region, the conserved region comprising one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

In some embodiments, a guide RNA (gRNA) is provided, the guide RNA comprising a guide region and a conserved region, the conserved region comprising one or more of:
(a) a shortened repeat/anti-repeat region, wherein the shortened repeat/anti-repeat region lacks 18-22 nucleotides relative to SEQ ID NO: 500, wherein
   (i) nucleotides 37-48 and 53-64 are deleted; and
   (ii) nucleotide 36 is linked to nucleotide 65 by 6-10 nucleotides; or
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500; or
(c) a shortened hairpin 2 region, wherein the shortened hairpin 2 lacks 18 nucleotides, wherein nucleotides 113-121 and 126-134 are deleted relative to SEQ ID NO: 500; and
wherein nucleotides 144-145 are deleted relative to SEQ ID NO: 500;
wherein at least 10 nucleotides are modified nucleotides.

The guide RNA (gRNA) of the previous embodiment comprising a guide region and a conserved region, the conserved region comprising:
(a) a shortened repeat/anti-repeat region, wherein the shortened repeat/anti-repeat region lacks 18-22 nucleotides, wherein
   (i) nucleotides 37-48 and 53-64 are deleted relative to SEQ ID NO: 500; and
   (ii) nucleotide 36 is linked to nucleotide 65 by 6-10 nucleotides;
(b) a shortened hairpin 1 region, wherein the shortened hairpin 1 lacks 2 nucleotides relative to SEQ ID NO: 500, wherein nucleotides 86 and 91 are deleted or nucleotides 85 and 92 are deleted;
(c) a shortened hairpin 2 region, wherein the shortened hairpin 2 lacks 18 nucleotides, wherein nucleotides 113-121 and 126-134 are deleted relative to SEQ ID NO: 500; and
(d) wherein nucleotides 144-145 are deleted relative to SEQ ID NO: 500;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 500. In further embodiments, nucleotide 36 is linked to nucleotide 65 by a sequence comprising the nucleotide sequence UGAAAC. In further embodiments, nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 950).

In some embodiments, the gRNA comprises a 5′ end modification. In some embodiments, the gRNA comprises a 3′ end modification. In some embodiments, the gRNA comprises a 5′ end modification and a 3′ end modification. In some embodiments, the gRNA comprises a modification in the upper stem region of the repeat/anti-repeat region. In some embodiments, the gRNA comprises a modification in the hairpin 1 region. In some embodiments, the gRNA comprises a modification in the hairpin 2 region.

In some embodiments, any of the foregoing modification is a modified nucleotide is selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide, optionally wherein the gRNA comprises at least two modifications independently selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide.

In some embodiments, the 5′ end modification comprises a modified nucleotide selected from (i) 2′-O-methyl (2′-OMe) modified nucleotide, (ii) 2′-O-(2-methoxyethyl) (2′-O-moe) modified nucleotide, (iii) a 2′-fluoro (2′-F) modified nucleotide, (iv) a phosphorothioate (PS) linkage between nucleotides, or (v) an inverted abasic modified nucleotide, optionally, wherein the gRNA comprises at least two 5′ end modifications independently selected from (i)-(v).

In some embodiments, the 3′ end modification comprises a modified nucleotide selected from (i) 2′-O-methyl (2′-OMe) modified nucleotide, (ii) 2′-O-(2-methoxyethyl) (2′-O-moe) modified nucleotide, (iii) a 2′-fluoro (2′-F) modified nucleotide, (iv) a phosphorothioate (PS) linkage between nucleotides, or (v) an inverted abasic modified nucleotide, optionally, wherein the gRNA comprises at least two 3′ end modifications independently selected from (i)-(v).

In some embodiments, the 5′ end modification comprises:
i. a modification of one or more of the first 1-4 nucleotides, wherein the modification is a PS linkage, inverted abasic nucleotide, 2′-OMe, 2′-O-moe, or 2′-F;
ii. a modification to the first nucleotide with 2′-OMe, 2′-O-moe, or 2′-F, and an optional one or two PS linkages to the next nucleotide or the first nucleotide of the 3′ tail;
iii. a modification to the first or second nucleotide with 2′-OMe, 2′-O-moe, or 2′-F, and optionally one or more PS linkages;
iv. a modification to the first, second, or third nucleotides with 2′-OMe, 2′-O-moe, or 2′-F, and optionally one or more PS linkages; or
v. a modification to the first, second, third, or forth nucleotides with 2′-OMe, 2′-O-moe, or 2′-F, and optionally one or more PS linkages,
   optionally, wherein the gRNA comprises at least two 5′ end modifications independently selected from (i)-(v).

The gRNA of any one of the preceding claims, wherein the 3′ end modification comprises:
i. a modification of one or more of the last 1-4 nucleotides, wherein the modification is a PS linkage, inverted abasic nucleotide, 2′-OMe, 2′-O-moe, or 2′-F;
ii. a modification to the last nucleotide with 2′-OMe, 2′-O-moe, or 2′-F, and an optional one or two PS linkages to the next nucleotide or the first nucleotide of the 3′ tail;
iii. a modification to the last or second to last nucleotide with 2′-OMe, 2′-O-moe, or 2′-F, and optionally one or more PS linkages;
iv. a modification to the last, second to last, or third to last nucleotides with 2′-OMe, 2′-O-moe, or 2′-F, and optionally one or more PS linkages; or
v. a modification to the last, second to last, third to last, or fourth to last nucleotides with 2′-OMe, 2′-O-moe, or 2′-F, and optionally one or more PS linkages,
   optionally wherein the gRNA comprises at least two 3′ end modifications independently selected from (i)-(v).

In some embodiments, the modification in the repeat/anti-repeat region, the hairpin 1 region, or the hairpin 2 region comprises a modified nucleotide selected from (i) 2′-O-methyl (2′-OMe) modified nucleotide, (ii) 2′-O-(2-methoxyethyl) (2′-O-moe) modified nucleotide, (iii) a 2′-fluoro (2′-F) modified nucleotide, or (iv) a phosphorothioate (PS) linkage between nucleotides, optionally wherein the modification in the repeat/anti-repeat region, the hairpin 1 region, or the hairpin 2 region comprises at least two modifications independently selected from (i)-(iv).

In some embodiments, the modification in the repeat/anti-repeat region, the hairpin 1 region, or the hairpin 2 region comprises a modified nucleotide selected from (i) 2′-O-methyl (2′-OMe) modified nucleotide, (ii) a 2′-fluoro (2′-F) modified nucleotide, or (iii) a phosphorothioate (PS) linkage between nucleotides, optionally wherein the repeat/anti-repeat region, the hairpin 1 region, or the hairpin 2 region comprises at least two modifications independently selected from (i)-(iii).

In some embodiments, the modification in the repeat/anti-repeat region, the hairpin 1 region, or the hairpin 2 region comprises a modified nucleotide selected from (i) 2′-O-methyl (2′-OMe) modified nucleotide, or (ii) a phosphorothioate (PS) linkage between nucleotides, optionally wherein the repeat/anti-repeat region, the hairpin 1 region, or the hairpin 2 region comprises at least two modifications independently selected from (i) and (ii).

In some embodiments, a composition comprising a gRNA associated with a lipid nanoparticle (LNP) disclosed herein is provided. In some embodiments, an LNP composition comprising a gRNA disclosed herein is provided. In some embodiments, the composition further comprises a nuclease or an mRNA which encodes the nuclease.

The following additional embodiments are provided herein.

Embodiment 1 is a guide RNA (gRNA) comprising a guide region and a conserved region, the conserved region comprising one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

Embodiment 2 is the gRNA of Embodiment 1, wherein the gRNA is a single-guide RNA (sgRNA) and wherein the gRNA comprises (a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 500; and
(ii) nucleotide 36 is linked to nucleotide 65 by at least 2 nucleotides.

Embodiment 3 is the gRNA of Embodiment 1 or 2, wherein the guide region has (i) an insertion of one nucleotide or a deletion of 1-4 nucleotides within positions 1-24 relative to SEQ ID NO: 500, or (ii) a length of 24 nucleotides.

Embodiment 4 is the gRNA of Embodiment 3, wherein the guide region has a length of 25, 24, 23, 22, 21, or 20 nucleotides, optionally wherein the guide region has a length of 25, 24, 23, or 22 nucleotides.

Embodiment 5 is the gRNA of Embodiment 4, wherein the guide region has a length of 23-24 nucleotides.

Embodiment 6 is the gRNA of any one of Embodiments 1-5, wherein the gRNA further comprises a 3′ tail.

Embodiment 7 is the gRNA of Embodiment 6, wherein the 3′ tail comprises 1, 2, 3, 4, 5, 6, 7, 8, 9, or 10 nucleotides.

Embodiment 8 is the gRNA of Embodiment 7, wherein the 3′ tail comprises 1, 2, 3, 4, or 5 nucleotides.

Embodiment 9 is the gRNA of any one of Embodiments 6-8, wherein the 3′ tail terminates with a nucleotide comprising a uracil or modified uracil.

Embodiment 10 is the gRNA of any one of Embodiments 6-9, wherein the 3′ tail is 1 nucleotide in length.

Embodiment 11 is the gRNA of any one of Embodiments 6-10, wherein the 3′ tail consists of a nucleotide comprising a uracil or a modified uracil.

Embodiment 12 is the gRNA of any one of Embodiments 6-11, wherein the 3′ tail comprises a modification of any one or more of the nucleotides present in the 3′ tail.

Embodiment 13 is the gRNA of any one of Embodiments 6-12, wherein the modification of the 3′ tail is one or more of 2′-O-methyl (2′-OMe) modified nucleotide and a phosphorothioate (PS) linkage between nucleotides.

Embodiment 14 is the gRNA of any one of Embodiments 6-13, wherein the 3′ tail is fully modified.

Embodiment 15 is the gRNA of any one of Embodiments 1-14, wherein the 3′ nucleotide of the gRNA is a nucleotide comprising a uracil or a modified uracil.

Embodiment 16 is the gRNA of any one of Embodiments 1-5, wherein one or more of nucleotides 144 and 145 are deleted relative to SEQ ID NO: 500.

Embodiment 17 is the gRNA of any one of Embodiments 1-5, wherein both nucleotides 144 and 145 are deleted relative to SEQ ID NO: 500.

Embodiment 18 is the gRNA of any one of Embodiments 1-5, wherein the gRNA does not comprise a 3′ tail.

Embodiment 19 is the gRNA of any one of Embodiments 1-18, wherein the shortened repeat/anti-repeat region lacks 2-24 nucleotides.

Embodiment 20 is the gRNA of any one of Embodiments 1-19, wherein the shortened repeat/anti-repeat region has a length of 28, 29, 30, 31, 32, 33, 34, 35, 36, 37, 38, 39, 40, 41, 42, 43, 44, 45, 46, 47, 48, 49, or 50 nucleotides.

Embodiment 21 is the gRNA of any one of Embodiments 1-20, wherein the shortened repeat/anti-repeat region lacks 12-24, optionally 18-24 nucleotides, optionally 20-22 nucleotides.

Embodiment 22 is the gRNA of any one of Embodiments 1-21, wherein the shortened repeat/anti-repeat region has a length of 28, 29, 30, 31, 32, 33, 34, 35, 36, 37, 38, 39, or 40 nucleotides.

Embodiment 23 is the gRNA of any one of Embodiments 1-22, wherein the shortened repeat/anti-repeat region has a length of 28, 29, 30, 31, 32, 33, or 34 nucleotides, or 30, 31, or 32 nucleotides.

Embodiment 24 is the gRNA of any one of Embodiments 1-23, wherein nucleotides 37-64 of SEQ ID NO: 500 form the upper stem, and one or more base pairs of the upper stem of the shortened repeat/anti-repeat region are deleted.

Embodiment 25 is the gRNA of any one of Embodiments 1-24, wherein the upper stem of the shortened repeat/anti-repeat region comprises no more than one, two, three, or four base pairs.

Embodiment 26 is the gRNA of any one of Embodiments 1-25, wherein all of positions 39-48 and all of positions 53-62 of the upper stem of the shortened repeat/anti-repeat region are deleted, and optionally nucleotide 38 or 63 is substituted.

Embodiment 27 is the gRNA of any one of Embodiments 1-26, wherein all of positions 38-48 and all of positions 53-63 of the upper stem of the shortened repeat/anti-repeat region are deleted, and optionally nucleotide 37 or 64 is substituted.

Embodiment 28 is the gRNA of any one of Embodiments 1-27, wherein all of nucleotides 37-48 and 53-64 of the upper stem of the shortened repeat/anti-repeat region are deleted, and optionally nucleotides 36 or 65 is substituted.

Embodiment 29 is the Grna of any one of Embodiments 1-28, wherein the shortened repeat/anti-repeat region has a duplex portion 11 base paired nucleotides in length.

Embodiment 30 is the gRNA of any one of Embodiments 1-29, wherein the shortened repeat/anti-repeat region has a single duplex portion.

Embodiment 31 is the gRNA of any one of Embodiments 1-29, wherein the shortened repeat/anti-repeat region has a first duplex portion and a second duplex portion.

Embodiment 32 is the gRNA of Embodiment 31, wherein the second duplex portion is 2-3 base paired nucleotides in length.

Embodiment 33 is the gRNA of Embodiment 31, wherein the first duplex portion is 11 base paired nucleotides in length and the second duplex portion is 3 base paired nucleotides in length.

Embodiment 34 is the gRNA of any one of Embodiments 1-33, wherein the upper stem of the shortened repeat/anti-repeat region includes one or more substitutions relative to SEQ ID NO: 500.

Embodiment 35 is the gRNA of any one of Embodiments 1-34, wherein one or more of nucleotides 49-52 is substituted relative to SEQ ID NO: 500.

Embodiment 36 is the gRNA of any one of Embodiments 1-33, wherein the shortened repeat/anti-repeat region is unsubstituted.

Embodiment 37 is the gRNA of any one of Embodiments 1-36, wherein the shortened repeat/anti-repeat region has 12-22 modified nucleotides

Embodiment 38 is the gRNA of Embodiment 37, wherein the shortened repeat/anti-repeat region does not comprise a modification at nucleotide 76.

Embodiment 39 is the gRNA of Embodiment 37, wherein the shortened repeat/anti-repeat does not comprise a phosphorothioate (PS) modification at nucleotide 76.

Embodiment 40 is the gRNA of any one of Embodiments 1-39, wherein the shortened hairpin 1 region lacks 2-10 nucleotides, optionally 2-8 or 2-4 nucleotides.

Embodiment 41 is the gRNA of any one of Embodiments 1-40, wherein the shortened hairpin 1 region has a length of 13, 14, 15, 16, 17, 18, 19, 20, or 21 nucleotides.

Embodiment 42 is the gRNA of Embodiment any one of Embodiments 1-41, wherein the shortened hairpin 1 region has a duplex portion 4-8, optionally 7-8 base paired nucleotides in length.

Embodiment 43 is the gRNA of Embodiment any one of Embodiments 1-41, wherein the shortened hairpin 1 region has a single duplex portion.

Embodiment 44 is the gRNA of any one of Embodiments 1-43, wherein one or two base pairs of the shortened hairpin 1 region are deleted.

Embodiment 45 is the gRNA of any one of Embodiments 1-44, wherein the stem of the shortened hairpin 1 region is seven or eight base paired nucleotides in length.

Embodiment 46 is the gRNA of any one of Embodiments 1-45, wherein one or more of positions 85-86 and one or more of nucleotides 91-92 of the shortened hairpin 1 region are deleted.

Embodiment 47 is the gRNA of any one of Embodiments 1-46, wherein nucleotides 86 and 91 or nucleotides 85 and 92 of the shortened hairpin 1 region are deleted.

Embodiment 48 is the gRNA of any one of Embodiments 1-47, wherein one or more of nucleotides 82-95 of the shortened hairpin 1 region is substituted relative to SEQ ID NO: 500.

Embodiment 49 is the gRNA of any one of Embodiments 1-48, wherein one or more of nucleotides 87-90 is substituted relative to SEQ ID NO: 500.

Embodiment 50 is the gRNA of any one of Embodiments 1-48, wherein the shortened hairpin 1 region is unsubstituted.

Embodiment 51 is the gRNA of any one of Embodiments 1-49, wherein the shortened hairpin 1 region has 6-15 modified nucleotides.

Embodiment 52 is the gRNA of any one of Embodiments 1-50, wherein the shortened hairpin 2 region lacks 2-18, optionally 2-16 nucleotides.

Embodiment 53 is the gRNA of any one of Embodiments 1-51, wherein the shortened hairpin 2 region has a length of 24, 25, 26, 27, 28, 29, 30, 31, 32, 33, 34, 35, 36, 37, 38, 39, or 40 nucleotides.

Embodiment 54 is the gRNA of any one of Embodiments 1-52, wherein the shortened hairpin 2 region has a length of 28, 29, 30, 31, 32, 33, or 34 nucleotides.

Embodiment 55 is the gRNA of any one of Embodiments 1-53, wherein one or more of nucleotides 113-121 and one or more of nucleotides 126-134 of the shortened hairpin 2 region are deleted.

Embodiment 56 is the gRNA of any one of Embodiments 1-54, wherein the shortened hairpin 2 region comprises an unpaired region.

Embodiment 57 is the gRNA of any one of Embodiments 1-55, wherein the shortened hairpin 2 region has two duplex portions.

Embodiment 58 is the gRNA of any one of Embodiments 1-56, wherein the shortened hairpin 2 region has a duplex portion of 4 base paired nucleotides in length.

Embodiment 59 is the gRNA of any one of Embodiments 57-58, wherein the shortened hairpin 2 region has a duplex portion of 4-8 base paired nucleotides in length.

Embodiment 60 is the gRNA of any one of Embodiments 57-59, wherein the shortened hairpin 2 region has a duplex portion of 4-6 base paired nucleotides in length.

Embodiment 61 is the gRNA of any one of Embodiments 1-60, wherein nucleotides 109-138 of SEQ ID NO: 500 form the upper stem, and the upper stem of the shortened hairpin 2 region comprises one, two, three, or four base pairs.

Embodiment 62 is the gRNA of any one of Embodiments 1-61, wherein at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted.

Embodiment 63 is the gRNA of any one of Embodiments 1-62, wherein all of positions 113-121 and 126-134 of the shortened hairpin 2 region are deleted.

Embodiment 64 is the gRNA of any one of Embodiments 1-63, wherein one or more of nucleotides 113-134 of the shortened hairpin 2 region is substituted relative to SEQ ID NO: 500.

Embodiment 65 is the gRNA of any one of Embodiments 1-64, wherein one or more of nucleotides 122-125 is substituted relative to SEQ ID NO: 500.

Embodiment 66 is the gRNA of any one of Embodiments 1-64, wherein the shortened hairpin 2 region is unsubstituted.

Embodiment 67 is the gRNA of Embodiment any one of Embodiments 1-66, wherein the shortened hairpin 2 region has 6-15 modified nucleotides.

Embodiment 68 is the gRNA of any one of Embodiments 1-67, wherein the guide region of the gRNA comprises at least two modified nucleotides, optionally at least four modified nucleotides.

Embodiment 69 is the gRNA of any one of Embodiments 1-68, wherein the guide region of the gRNA comprises 1, 2, 3, 4, 5, 6, 7, 8, 9, 10, 11, or 12 modified nucleotides, optionally 1, 2, or 3 modified nucleotides.

Embodiment 70 is the gRNA of any one of Embodiments 1-69, wherein the guide region of the gRNA comprises 4, 5, 6, 7, 8, 9, 10, 11, or 12 modified nucleotides.

Embodiment 71 is the gRNA of any one of Embodiments 1-70, wherein the guide region of the gRNA comprises 6, 7, 8, 9, 10, 11, or 12 modified nucleotides.

Embodiment 72 is the gRNA of any one of Embodiments 1-71, wherein the guide region does not comprise a modified nucleotide 3′ of the first three nucleotides of the guide region.

Embodiment 73 is the gRNA of any one of Embodiments 1-66, wherein the guide region does not comprise a modified nucleotide.

Embodiment 74 is the gRNA of any one of Embodiments 1-72, wherein the gRNA comprises a 5′ end modification.

Embodiment 75 is the gRNA of any one of Embodiments 1-74, wherein the gRNA comprises a 3′ end modification.

Embodiment 76 is the gRNA of any one of Embodiments 1-75, wherein the gRNA comprises a 5′ end modification and a 3′ end modification.

Embodiment 77 is the gRNA of any one of Embodiments 1-76, comprising a modification in the upper stem region of the repeat/anti-repeat region.

Embodiment 78 is the gRNA of any one of Embodiments 1-77, comprising a modification in the hairpin 1 region.

Embodiment 79 is the gRNA of any one of Embodiments 1-78, comprising a modification in the hairpin 2 region.

Embodiment 80 is the gRNA of Embodiment 79, wherein the modification in the hairpin 2 region comprises a modification at 1, 2, 3, or 4 nucleotides of nucleotides 106-109.

Embodiment 81 is the gRNA of Embodiment 80, wherein the modification in the hairpin 2 region comprises a modification at each of nucleotides 106-109.

Embodiment 82 is the gRNA of any one of Embodiments 80 or 81, wherein the modification comprises a 2′-O-methyl (2′-O-Me) modification.

Embodiment 83 is the gRNA of any one of Embodiments 1-82, comprising a 3′ end modification, and comprising a modification in the upper stem region of the repeat/anti-repeat region.

Embodiment 84 is the gRNA of any one of Embodiments 1-83, comprising a 3′ end modification, and a modification in the hairpin 1 region.

Embodiment 85 is the gRNA of any one of Embodiments 1-83, comprising a 3′ end modification, and a modification in the hairpin 2 region.

Embodiment 86 is the gRNA of any one of Embodiments 1-85, comprising a 5′ end modification, and comprising a modification in the upper stem region of the repeat/anti-repeat region.

Embodiment 87 is the gRNA of any one of Embodiments 1-86, comprising a 5′ end modification, and a modification in the hairpin 1 region.

Embodiment 88 is the gRNA of any one of Embodiments 1-87, comprising a 5′ end modification, and a modification in the hairpin 2 region.

Embodiment 89 is the gRNA of any one of Embodiments 1-88, comprising a 5′ end modification, a modification in the upper stem region of the repeat/anti-repeat region, and a 3′ end modification.

Embodiment 90 is the gRNA of any one of Embodiments 1-89, comprising a 5′ end modification, a modification in the hairpin 1 region, and a 3′ end modification.

Embodiment 91 is the gRNA of any one of Embodiments 1-90, comprising a 5′ end modification, a modification in the hairpin 1 region, a modification in the hairpin 2 region, and a 3′ end modification.

Embodiment 92 is the gRNA of any one of Embodiments 1-91, comprising a 5′ end modification, a modification in the repeat/anti-repeat region, a modification in the hairpin 1 region, a modification in the hairpin 2 region, and a 3′ end modification.

Embodiment 93 is the gRNA of any one of Embodiments 1-92, wherein the modification in the repeat/anti-repeat region does not comprise a phosphorothioate (PS) modification at nucleotide 76.

Embodiment 94 is the gRNA of any one of Embodiments 1-93, wherein the modification in the repeat/anti-repeat region does not comprise a modification at nucleotide 76.

Embodiment 95 is the gRNA of any one of Embodiments 74-94, wherein the 5′ end modification comprises a modified nucleotide selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

Embodiment 96 is the gRNA of any one of the Embodiments 74-95, wherein the 3′ end modification comprises a modified nucleotide selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

Embodiment 97 is the gRNA of any one of the Embodiments 74-96, wherein the 5′ end modification comprises any of.
i, a modification of any one or more of the first 1, 2, 3, or 4 nucleotides;
ii. one modified nucleotide;
iii. two modified nucleotides;
iv. three modified nucleotides; and
v. four modified nucleotides.

Embodiment 98 is the gRNA of any one of Embodiments 74-97, wherein the 5′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide; and
v. an inverted abasic modified nucleotide.

Embodiment 99 is the gRNA of any one of Embodiments 74-98, wherein the 3′ end modification comprises any of:
i. a modification of any one or more of the last 4, 3, 2, or 1 nucleotides;
ii. one modified nucleotide;
iii. two modified nucleotides;
iv. three modified nucleotides; and
v. four modified nucleotides.

Embodiment 100 is the gRNA of any one of Embodiments 74-99, wherein the 3′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide; and
v. an inverted abasic modified nucleotide.

Embodiment 101 is the gRNA of any one of Embodiments 74-100, wherein the 5′ end modification comprises at least one PS linkage, and wherein one or more of:
i. there is one PS linkage, and the linkage is between the first and second nucleotides;
ii. there are two PS linkages between the first three nucleotides;
iii. there are PS linkages between any one or more of the first four nucleotides; and
iv. there are PS linkages between any one or more of the first five nucleotides.

Embodiment 102 is the gRNA of Embodiment 101, wherein the 5′ end modification further comprises at least one 2′-OMe, 2′-O-moe, inverted abasic, or 2′-F modified nucleotide.

Embodiment 103 is the gRNA of any one of Embodiments 1-102, wherein the 5′ end modification comprises:
i. a modification of one or more of the first 1-4 nucleotides, wherein the modification is a PS linkage, inverted abasic nucleotide, 2′-OMe, 2′-O-moe, or 2′-F;
ii. a modification to the first nucleotide with 2′-Ome, 2′-O-moe, or 2′-F, and an optional one or two PS linkages to the next nucleotide or the first nucleotide of the 3′ tail;
iii. a modification to the first or second nucleotide with 2′-Ome, 2′-O-moe, or 2′-F, and optionally one or more PS linkages;
iv. a modification to the first, second, or third nucleotides with 2′-Ome, 2′-O-moe, or 2′-F, and optionally one or more PS linkages; or
v. a modification to the first, second, third, or forth nucleotides with 2′-Ome, 2′-O-moe, or 2′-F, and optionally one or more PS linkages.

Embodiment 104 is the gRNA of any one of Embodiments 1-103, wherein the 3′ end modification comprises at least one PS linkage, and wherein one or more of:
i. there is one PS linkage, and the linkage is between the last and second to last nucleotides;
ii. there are two PS linkages between the last three nucleotides; and
iii. there are PS linkages between any one or more of the last four nucleotides.

Embodiment 105 is the gRNA of Embodiment 104, wherein the 3′ end modification further comprises at least one 2′-Ome, 2′-O-moe, inverted abasic, or 2′-F modified nucleotide.

Embodiment 106 is the gRNA of any one of Embodiments 1-105, wherein the 3′ end modification comprises:
i. a modification of one or more of the last 1-4 nucleotides, wherein the modification is a PS linkage, inverted abasic nucleotide, 2′-OMe, 2′-O-moe, or 2′-F;
ii. a modification to the last nucleotide with 2′-OMe, 2′-O-moe, or 2′-F, and an optional one or two PS linkages to the next nucleotide or the first nucleotide of the 3′ tail;
iii. a modification to the last or second to last nucleotide with 2′-OMe, 2′-O-moe, or 2′-F, and optionally one or more PS linkages;
iv. a modification to the last, second to last, or third to last nucleotides with 2′-OMe, 2′-O-moe, or 2′-F, and optionally one or more PS linkages; or
v. a modification to the last, second to last, third to last, or fourth to last nucleotides with 2′-OMe, 2′-O-moe, or 2′-F, and optionally one or more PS linkages.

Embodiment 107 is the gRNA of any one of Embodiments 1-106, wherein the modification in the repeat/anti-repeat region, the hairpin 1 region, or the hairpin 2 region comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, or a phosphorothioate (PS) linkage between nucleotides.

Embodiment 108 is the gRNA of any one of Embodiments 1-106, wherein the modification in the repeat/anti-repeat region, the hairpin 1 region, or the hairpin 2 region comprises a modified nucleotide selected from 2′-O-methyl (2′-OMe) modified nucleotide, a 2′-fluoro (2′-F) modified nucleotide, or a phosphorothioate (PS) linkage between nucleotides.

Embodiment 109 is the gRNA of any one of Embodiments 1-106, wherein the modification in the repeat/anti-repeat region, the hairpin 1 region, or the hairpin 2 region comprises a modified nucleotide selected from 2′-O-methyl (2′-OMe) modified nucleotide or a phosphorothioate (PS) linkage between nucleotides.

Embodiment 110 is the gRNA of any one of Embodiments 1-109, wherein the modification in the repeat/anti-repeat region does not comprise a phosphorothioate modification at nucleotide 76.

Embodiment 111 is the gRNA of any one of Embodiments 1-110, wherein the modification in the repeat/anti-repeat region does not comprise a modification at nucleotide 76.

Embodiment 112 is the gRNA of any one of Embodiments 1-111, wherein at least 20%, 30%, 40%, or 50% of the nucleotides are modified nucleotides.

Embodiment 113 is the gRNA of Embodiment 112, wherein the gRNA comprises modified nucleotides selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or combinations thereof.

Embodiment 114 is the gRNA of any one of Embodiments 1-113, wherein the modification comprises a modification at 1, 2, 3, or 4 nucleotides of nucleotides 106-109.

Embodiment 115 is the gRNA of any one of Embodiments 113 or 114, wherein the modification comprises a modification at each of nucleotides 106-109.

Embodiment 116 is the gRNA of any one of Embodiments 114-115, wherein the modification comprises a 2′-O-methyl modification.

Embodiment 117 is the gRNA of any one of Embodiments 112-116, wherein the gRNA comprises modified nucleotides selected from 2′-O-methyl (2′-Ome) modified nucleotide, a 2′-fluoro (2′-F) modified nucleotide, a phosphorothioate (PS) linkage between nucleotides, or combinations thereof.

Embodiment 118 is the gRNA of any one of Embodiments 1-117, wherein nucleotides 1-3 of the guide region are modified and nucleotides in the guide region other than nucleotides 1-3 are not modified.

Embodiment 119 is the gRNA of any one of Embodiments 1-118, wherein a 3′ tail of nucleotide 144 is present in the gRNA, and comprises 2′-O-Me modified nucleotides at nucleotides 141-144 and two PS linkages between nucleotides 141-142 and 142-143 respectively.

Embodiment 120 is the gRNA of any one of Embodiments 1-119, wherein one or more positions of 49-52, 87-90, or 122-125 is substituted.

Embodiment 121 is a single guide RNA (sgRNA) comprising any one of SEQ ID NOs: 1-19 and 21-42.

Embodiment 122 is the gRNA of any one of Embodiments 1-121, comprising a nucleotide sequence having at least 99, 98, 97, 96, 95, 94, 93, 92, 91, 90, 85, 80, 75, or 70% identity to the nucleotide sequence of any one of SEQ ID Nos: 1-19 and 21-42.

Embodiment 123 is the gRNA of any one of Embodiments 1-121, comprising a nucleotide sequence having at least 99, 98, 97, 96, 95, 94, 93, 92, 91, 90, 85, 80, 75, or 70% identity to the nucleotide sequence of any one of SEQ ID Nos: 1-19 and 21-42, wherein the modification at each nucleotide of the gRNA that corresponds to a nucleotide of the reference sequence identifier in Table 1 is identical to or equivalent to the modification shown in the reference sequence identifier in Table 2.

Embodiment 124 is the gRNA of any one of Embodiments 1-122, comprising a nucleotide sequence having at least 99, 98, 97, 96, 95, 94, 93, 92, 91, or 90% identity to the sequence from X to the 3′ end of the nucleotide sequence of any one of SEQ ID Nos: 1-5, 7, 8, 11, 12, 13, 15, 16, 18, 19, 21, 23, 24, 26, 27, 28, 30, 31, 33, 34, 35, 37, 39, 41, 101-291, 301-494, 931-946, 951, and 952, where X is the first nucleotide of the conserved region.

Embodiment 125 is the gRNA of any one of Embodiments 121-124, further comprising a 3′ tail comprising a 2′-O-Me modified nucleotide.

Embodiment 126 is the gRNA of any one of Embodiments 1-125, wherein the gRNA directs a nuclease to a target sequence for binding.

Embodiment 127 is the gRNA of any one of Embodiments 1-126, wherein the gRNA directs a nuclease to a target sequence for inducing a double-strand break within the target sequence.

Embodiment 128 is the gRNA of any one of Embodiments 1-127, wherein the gRNA directs a nuclease to a target sequence for inducing a single-strand break within the target sequence.

Embodiment 129 is the gRNA of any one of Embodiments 126-129, wherein the nuclease is a Nine Cas9.

Embodiment 130 is the gRNA of any one of Embodiments 1-129, wherein the gRNA comprises a conservative substitution, optionally wherein the conservative substitution maintains at least one base pair.

Embodiment 131 is a composition comprising a gRNA of any one of Embodiments 1-130, associated with a lipid nanoparticle (LNP).

Embodiment 132. An LNP composition comprising a gRNA of any one of Embodiments 1-130.

Embodiment 133 is a composition comprising the gRNA of any one of Embodiments 1-130, or the composition of any one of Embodiments 131-132, further comprising a nuclease or an mRNA which encodes the nuclease.

Embodiment 134 is the composition of Embodiment 133, wherein the nuclease is a Cas protein.

Embodiment 135 is the composition of Embodiment 134, wherein the Cas protein is a Nme Cas9.

Embodiment 136 is the composition of Embodiment 135, wherein the Nine Cas9 is an Nme1 Cas9, an Nme2 Cas9, or an Nme3 Cas9.

Embodiment 137 is the composition of any one of Embodiments 133-136, wherein the nuclease has a double strand cleaving activity.

Embodiment 138 is the composition of any one of Embodiments 133-137, wherein the nuclease has a nickase activity.

Embodiment 139 is the composition of any one of Embodiments 133-138, wherein the nuclease has a dCas DNA binding domain.

Embodiment 140 is the composition of any one of Embodiments 133-139, wherein the nuclease is modified.

Embodiment 141 is the composition of Embodiment 140, wherein the modified nuclease comprises a heterologous functional domain.

Embodiment 142 is the composition of Embodiment 141, wherein the heterologous functional domain is a deaminase.

Embodiment 143 is the composition of Embodiment 142, further comprising a UGI or a mRNA encoding a UGI.

Embodiment 144 is the composition of any one of Embodiments 142-143, wherein the heterologous functional domain is a cytidine deaminase.

Embodiment 145 is the composition of any one of Embodiments 140-144, wherein the modified nuclease comprises a nuclear localization signal (NLS).

Embodiment 146 is the composition of any one of Embodiments 133-145, comprising an mRNA which encodes the nuclease.

Embodiment 147 is the composition of Embodiment 146, wherein the mRNA comprises the sequence of any one of SEQ ID NOs: 636-638.

Embodiment 148 is a pharmaceutical formulation comprising the gRNA of any one of Embodiments 1-130 or the composition of any one of Embodiments 131-147 and a pharmaceutically acceptable carrier.

Embodiment 149 is a method of modifying a target DNA comprising, delivering a Cas protein or a nucleic acid encoding a Cas protein, and any one or more of the following to a cell:
i. the gRNA of any one of Embodiments 1-130;
ii. the composition of any one of Embodiments 131-147; and
iii. the pharmaceutical formulation of Embodiment 148.

Embodiment 150 is the method of Embodiment 149, wherein the method results in an insertion or deletion in a gene.

Embodiment 151 is the method of Embodiment 149 or 150, wherein the method results in at least one base edit.

Embodiment 152 is the method of any one of Embodiments 149-151, further comprising delivering to the cell a template, wherein at least a part of the template incorporates into a target DNA at or near a double strand break site induced by the Cas protein.

Embodiment 153 is the gRNA of any one of Embodiments 1-130, the composition of Embodiments 131-147, or the pharmaceutical formulation of Embodiment 148 for use in preparing a medicament for treating a disease or disorder.

Embodiment 154 is use of the gRNA of any one of Embodiments 1-130, the composition of Embodiments 131-147, or the pharmaceutical formulation of Embodiment 148 in the manufacture of a medicament for treating a disease or disorder.

### FIGURE LEGENDS

FIG. 1 shows mean editing results with standard deviation in HEK-Blue™ cells using truncated gRNAs.

FIG. 2 shows mean percent editing results for dual guide RNA (dgRNA) targeting VEGFA in HEK-Nme2 cells.

FIG. 3 shows the mean percent editing results of chemically modified sgRNA in HEK-Nme2 cells targeting the VEGFA gene at site T47.

FIG. 4 shows the mean percent editing results of modified sgRNA in HEK-293 cells targeting the VEGFA gene at site T47.

FIG. 5 shows mean percent editing at the TTR locus in PMH with increasing doses of Nme2Cas9 mRNA and chemically modified sgRNA.

FIG. 6 shows mean percent editing at PCSK9 locus in PMH with modified sgRNAs.

FIG. 7 shows mean percent editing in PMH of several Nme2Cas9 mRNAs with a modified sgRNA.

FIG. 8A shows mean percent editing at the TTR locus in PMH using varying ratios of sgRNA and Nme2Cas9 mRNA.

FIG. 8B shows mean percent editing at the TTR locus in PMH using varying ratios a pgRNA and Nme2Cas9 mRNA.

FIG. 9 shows mean percent editing at the TTR locus in PMH for pgRNAs with Nme2Cas9 mRNA.

FIG. 10A shows mean percent editing at the VEGFA TS-25 locus in HEK-Nme2 cells for combinations of modified crRNAs and trRNAs with Nme2Cas9 mRNA.

FIG. 10B shows mean percent editing at the VEGFA TS-47 locus in HEK-Nme2 cells for combinations of modified crRNAs and trRNAs with Nme2Cas9 mRNA.

FIG. 11 shows mean percent editing at the VEGFA TS-47 locus in HEK-Nme2 cells dgRNAs consisting of different crRNA and tracrRNA combinations for combinations of modified crRNAs and trRNAs with Nme2Cas9 mRNA.

FIG. 12A shows mean percent editing at TTR exon 1 in PMH for pgRNAs with 2′-OMe modification in the guide sequence.

FIG. 12B shows mean percent editing at TTR exon 3 in PMH for pgRNAs with 2′-OMe modification in the guide sequence.

FIG. 12C shows mean percent editing at TTR exon 1 in PMH for pgRNAs with light 2′-OMe modification in the guide sequence.

FIG. 12D shows mean percent editing at TTR exon 3 in PMH for pgRNAs with light 2′-OMe modification in the guide sequence.

FIG. 13 shows mean editing percentage in at the PCSK9 locus in PMH.

FIG. 14A shows mean editing results at the VEGFA locus in HEK cells treated with mRNA C (SEQ ID NO: 622).

FIG. 14B shows mean editing results at the VEGFA locus in HEK cells treated with mRNA I (SEQ ID NO: 627).

FIG. 14C shows mean editing results at the VEGFA locus in HEK cells treated with mRNA J (SEQ ID NO: 628).

FIG. 14D shows mean editing results at the VEGFA locus in PHH cells treated with mRNA C (SEQ ID NO: 622).

FIG. 14E shows mean editing results at the VEGFA locus in PHH cells treated with mRNA I (SEQ ID NO: 627).

FIG. 14F shows mean editing results at the VEGFA locus in PHH cells treated with mRNA J (SEQ ID NO: 628).

FIG. 15 shows mean percent editing at the mouse TTR locus in PMH cells treated with NmeCas9 constructs designed with 1 or 2 nuclear localization sequences.

FIG. 16 shows mean percent editing at the mouse TTR locus in PMH cells treated with pgRNA and various Nme2Cas9 mRNAs.

FIG. 17 shows fold change in Nme2Cas9 protein expression compared to SpyCas9 protein expression in PMH, PRH, PCH and PHH cells.

FIGS. 18A-18F show fold change in Nme2Cas9 protein expression compared to SpyCas9 protein expression in T cells from 2 donors assayed at 24 hours, 48 hours and 72 hours after treatment.

FIG. 19 shows mean percent editing at the TTR locus in mouse liver treated with sgRNA and Nme2Cas9.

FIG. 20A shows mean percent editing at the TTR locus in mouse liver following treatment with pgRNA and Nme2Cas9.

FIG. 20B shows mean serum TTR protein following treatment with pgRNA and Nme2Cas9.

FIG. 20C shows mean percent TTR knockdown following treatment with pgRNA and Nme2Cas9.

FIG. 20D shows mean percent editing at the TTR locus in mouse liver following treatment with pgRNA and various Nme2Cas9.

FIG. 20E shows serum TTR protein knockdown following treatment with pgRNA and various Nme2Cas9.

FIG. 21 shows mean percent editing in mouse liver following treatment with various Nme2Cas9 constructs.

FIG. 22 shows mean percent editing in mouse liver following treatment with pgRNA and various Nme2Cas9

FIG. 23 shows mean percent editing in mouse liver following treatment with various base editors.

FIG. 24 shows an exemplary schematic of Nme2 sgRNA in a possible secondary structure, including the repeat/anti-repeat region and the hairpin region which includes hairpin 1 and hairpin 2 regions and further indicates the guide region (or targeting region) (denoted with a gray fill with dashed outline), bases not amenable to single or pairwise deletion (denoted with a gray fill with solid outline), bases amenable to single or pairwise deletion (open circles).

FIG. 25 shows an exemplary sgRNA (G021536; SEQ ID NO: 490) in a possible secondary structure. The methylation is shown in bold; phosphorothioate linkages are indicated by ‘*’. Watson-Crick base pairing is indicated by a ‘-’ between nucleotides in duplex portions. Non-Watson-Crick base pairing is indicated by a ‘•’ between nucleotides in duplex portions.

FIG. 26 shows the percent editing at the TTR locus in primary mouse hepatocytes.

FIG. 27 shows serum TTR levels in mice.

FIG. 28 shows percent editing at the TTR locus in mouse liver samples.

FIG. 29 shows serum TTR measurements following treatment in mice.

FIG. 30 shows percent editing at the TTR locus in mouse liver samples.

FIG. 31 shows the mean percent CD3 negative T cells following TRAC editing with Nme1Cas9.

FIG. 32 shows the mean percent CD3 negative T cells following TRAC editing with Nme3Cas9.

FIG. 33 shows the expression of Nme-HiBiT constructs in T cells at 24 hours.

FIG. 34 shows the CD3-negative cell population as a function of NmeCas9 mRNA amount.

FIG. 35 shows the dose response curve for select gRNAs in PCH.

FIG. 36 shows the dose response curve for LNP dilution series in PCH.

FIG. 37 shows an exemplary sgRNA (Guide ID G032572; SEQ ID NO: 951) in a possible secondary structure. The unmodified nucleotides are shown in bold and methylation is shown in light fonts; phosphorothioate linkages are indicated by ‘*’. Watson-Crick base pairing is indicated by a ‘-’ between nucleotides in duplex portions. Non-Watson-Crick base pairing is indicated by a ‘•’ between nucleotides in duplex portions.

FIG. 38 shows an exemplary sgRNA (Guide ID G031771; SEQ ID NO: 952) in a possible secondary structure. The unmodified nucleotides are shown in bold and methylation is shown in light fonts; phosphorothioate linkages are indicated by ‘*’. Watson-Crick base pairing is indicated by a ‘-’ between nucleotides in duplex portions. Non-Watson-Crick base pairing is indicated by a ‘•’ between nucleotides in duplex portions.

FIG. 39 shows serum TTR levels in mice.

FIG. 40 shows percent editing at the TTR locus in mouse liver samples.

FIG. 41 shows the dose response curve for select gRNAs in PMH.

FIG. 42 shows the dose response curve for select gRNAs in PMH.

### DETAILED DESCRIPTION

Provided herein are shortened gRNAs for use in gene editing methods. Examples of sequences of engineered and tested gRNAs are shown in Tables 1-2.

Certain of the gRNAs provided herein are single guide RNAs (sgRNAs) for use in gene editing methods.

Certain of the gRNAs provided herein are dual guide RNAs (dgRNAs) for use in gene editing methods.

This disclosure further provides exemplary uses of these gRNAs to alter the genome of a target nucleic acid in vitro (e.g., cells cultured in vitro for use in ex vivo therapy or other uses of genetically edited cells) or in a cell in a subject such as a human (e.g., for use in in vivo therapy).

TABLE 1
Exemplary Sequences for gRNAs

| | SEQ | |
|---|---|---|
| Length | ID | |
| (nt) | NO: | Sequence |
| 145 | 500 | NNNNNNNNNNNNNNNNNNNNNNNNGUUGUA |
| | | GCUCCCUUUCUCAUUUCGGAAACGAAAUGA |
| | | GAACCGUUGCUACAAUAAGGCCGUCUGAAA |
| | | AGAUGUGCCGCAACGCUCUGCCCCUUAAAG |
| | | CUUCUGCUUUAAGGGGCAUCGUUUA |
| 101 | 1 | (N)₂₀₋₂₅GUUGUAGCUCCCUGAAACCGUU |
| | | GCUACAAUAAGGCCGUCGAAAGAUGUGCCG |
| | | CAACGCUCUGCCUUCUGGCAUCGUU |
| 105 | 2 | (N)₂₀₋₂₅GUUGUAGCUCCCUGAAACCGUU |
| | | GCUACAAUAAGGCCGUCGAAAGAUGUGCCG |
| | | CAACGCUCUGCCUUCUGGCAUCGUUUAUU |
| 107 | 3 | (N)₂₀₋₂₅GUUGUAGCUCCCUGGAAACCCG |
| | | UUGCUACAAUAAGGCCGUCGAAAGAUGUGC |
| | | CGCAACGCUCUGCCUUCUGGCAUCGUUUAU |
| | | U |
| 101 | 4 | mN*mNNNNNNNNmNNNmNNNNNNNNNNNNm |
| | | GUUGmUmAmGmCUCCCmUmGmAmAmAmCmC |
| | | GUUmGmCUAmCAAU*AAGmGmCCmGmUmCm |
| | | GmAmAmAmGmAmUGUGCmCGCmAmAmCmGC |
| | | UCUmGmCCmUmUmCmUGmGCmAmUC*mG*m |
| | | U*mU |
| 97-102 | 5 | (N)₂₀₋₂₅mGUUGmUmAmGmCUCCCmUmGm |
| | | AmAmAmCmCGUUmGmCUAmCAAU*AAGmGm |
| | | CCmGmUmCmGmAmAmAmGmAmUGUGCmCGC |
| | | mAmAmCmGCUCUmGmCCmUmUmCmUGmGCm |
| | | AmUC*mG*mU*mU |
| 77 | 6 | mGUUGmUmAmGmCUCCCmUmGmAmAmAmCm |
| | | CGUUmGmCUAmCAAU*AAGmGmCCmGmUmC |
| | | mGmAmAmAmGmAmUGUGCmCGCmAmAmCmG |
| | | CUCUmGmCCmUmUmCmUGmGCmAmUC*mG* |
| | | mU*mU |
| 101 | 7 | mN*mN*mN*mNmNNNmNmNNmNNmNNNNNm |
| | | NNNNmNNNmGUUGmUmAmGmCUCCCmUmGm |
| | | AmAmAmCmCGUUmGmCUAmCAAU*AAGmGm |
| | | CCmGmUmCmGmAmAmAmGmAmUGUGCmCGm |
| | | CAAmCGCUCUmGmCCmUmUmCmUGGCAUCG |
| | | *mU*mU |
| 97-102 | 8 | (N)₂₀₋₂₅mGUUGmUmAmGmCUCCCmUmGm |
| | | AmAmAmCmCGUUmGmCUAmCAAU*AAGmGm |
| | | CCmGmUmCmGmAmAmAmGmAmUGUGCmCGm |
| | | CAAmCGCUCUmGmCCmUmUmCmUGGCAUCG |
| | | *mU*mU |
| 77 | 9 | mGUUGmUmAmGmCUCCCmUmGmAmAmAmCm |
| | | CGUUmGmCUAmCAAU*AAGmGmCCmGmUmC |
| | | mGmAmAmAmGmAmUGUGCmCGmCAAmCGCU |
| | | CUmGmCCmUmUmCmUGGCAUCG*mU*mU |
| 105 | 10 | (N)₂₀₋₂₅GUUGUAGCUCCCUUCGAAAGAC |
| | | CGUUGCUACAAUAAGGCCGUCGAAAGAUGU |
| | | GCCGCAACGCUCUGCCUUCUGGCAUCGUU |
| 105 | 11 | mN*mN*mN*mNmNNNmNmNNmNNmNNNNNm |
| | | NNNNmNNNmGUUGmUmAmGmCUCCCmUmUm |
| | | CmGmAmAmAmGmAmCmCGUUmGmCUAmCAA |
| | | U*AAGmGmCCmGmUmCmGmAmAmAmGmAmU |
| | | GUGCmCGmCAAmCGCUCUmGmCCmUmUmCm |
| | | UGGCAUCG*mU*mU |
| 101 | 12 | mN*mNNNNNNNNmNNNmNNNNNNNNNNNNm |
| | | GUUGmUmAmGmCUCCCmUmGmAmAmAmCmC |
| | | GUUmGmCUAmCAAUAAGmGmCCmGmUmCmG |
| | | mAmAmAmGmAmUGUGCmCGCmAmAmCmGCU |
| | | CUmGmCCmUmUmCmUGmGCmAmUC*mG*mU |
| | | *mU |
| 97-102 | 13 | (N)₂₀₋₂₅mGUUGmUmAmGmCUCCCmUmGm |
| | | AmAmAmCmCGUUmGmCUAmCAAUAAGmGmC |
| | | CmGmUmCmGmAmAmAmGmAmUGUGCmCGCm |
| | | AmAmCmGCUCUmGmCCmUmUmCmUGmGCmA |
| | | mUC*mG*mU*mU |
| 77 | 14 | mGUUGmUmAmGmCUCCCmUmGmAmAmAmCm |
| | | CGUUmGmCUAmCAAUAAGmGmCCmGmUmCm |
| | | GmAmAmAmGmAmUGUGCmCGCmAmAmCmGC |
| | | UCUmGmCCmUmUmCmUGmGCmAmUC*mG*m |
| | | U*mU |
| 101 | 15 | mN*mN*mN*mNmNNNmNmNNmNNmNNNNNm |
| | | NNNNmNNNmGUUGmUmAmGmCUCCCmUmGm |
| | | AmAmAmCmCGUUmGmCUAmCAAUAAGmGmC |
| | | CmGmUmCmGmAmAmAmGmAmUGUGCmCGmC |
| | | AAmCGCUCUmGmCCmUmUmCmUGGCAUCG* |
| | | mU*mU |
| 97-102 | 16 | (N)₂₀₋₂₅mGUUGmUmAmGmCUCCCmUmGm |
| | | AmAmAmCmCGUUmGmCUAmCAAUAAGmGmC |
| | | CmGmUmCmGmAmAmAmGmAmUGUGCmCGmC |
| | | AAmCGCUCUmGmCCmUmUmCmUGGCAUCG* |
| | | mU*mU |
| 77 | 17 | mGUUGmUmAmGmCUCCCmUmGmAmAmAmCm |
| | | CGUUmGmCUAmCAAUAAGmGmCCmGmUmCm |
| | | GmAmAmAmGmAmUGUGCmCGmCAAmCGCUC |
| | | UmGmCCmUmUmCmUGGCAUCG*mU*mU |
| 105 | 18 | mN*mN*mN*mNmNNNmNmNNmNNmNNNNNm |
| | | NNNNmNNNmGUUGmUmAmGmCUCCCmUmUm |
| | | CmGmAmAmAmGmAmCmCGUUmGmCUAmCAA |
| | | UAAGmGmCCmGmUmCmGmAmAmAmGmAmUG |
| | | UGCmCGmCAAmCGCUCUmGmCCmUmUmCmU |
| | | GGCAUCG*mU*mU |
| 101 | 19 | mN*mNNNNNNNNmNNNmNNNNNNNNNNNNm |
| | | GUUGmUmAmGmCUCCCmUmGmAmAmAmCmC |
| | | GUUmGmCUAmCAAU*AAGmGmCCmGmUmCm |
| | | GmAmAmAmGmAmUGUGCmCGCmAmAmCmGm |
| | | CmUmCmUmGmCCmUmUmCmUGmGCmAmUC* |
| | | mG*mU*mU |
| 97-102 | 21 | (N)₂₀₋₂₅mGUUGmUmAmGmCUCCCmUmGm |
| | | AmAmAmCmCGUUmGmCUAmCAAU*AAGmGm |
| | | CCmGmUmCmGmAmAmAmGmAmUGUGCmCGC |
| | | mAmAmCmGmCmUmCmUmGmCCmUmUmCmUG |
| | | mGCmAmUC*mG*mU*mU |
| 77 | 22 | mGUUGmUmAmGmCUCCCmUmGmAmAmAmCm |
| | | CGUUmGmCUAmCAAU*AAGmGmCCmGmUmC |
| | | mGmAmAmAmGmAmUGUGCmCGCmAmAmCmG |
| | | mCmUmCmUmGmCCmUmUmCmUGmGCmAmUC |
| | | *mG*mU*mU |
| 101 | 23 | mN*mN*mN*mNmNNNmNmNNmNNmNNNNNm |
| | | NNNNmNNNmGUUGmUmAmGmCUCCCmUmGm |
| | | AmAmAmCmCGUUmGmCUAmCAAU*AAGmGm |
| | | CCmGmUmCmGmAmAmAmGmAmUGUGCmCGm |
| | | CAAmCGmCmUmCmUmGmCCmUmUmCmUGGC |
| | | AUCG*mU*mU |
| 97-102 | 24 | (N)₂₀₋₂₅mGUUGmUmAmGmCUCCCmUmGm |
| | | AmAmAmCmCGUUmGmCUAmCAAU*AAGmGm |
| | | CCmGmUmCmGmAmAmAmGmAmUGUGCmCGm |
| | | CAAmCGmCmUmCmUmGmCCmUmUmCmUGGC |
| | | AUCG*mU*mU |
| 77 | 25 | mGUUGmUmAmGmCUCCCmUmGmAmAmAmCm |
| | | CGUUmGmCUAmCAAU*AAGmGmCCmGmUmC |
| | | mGmAmAmAmGmAmUGUGCmCGmCAAmCGmC |
| | | mUmCmUmGmCCmUmUmCmUGGCAUCG*mU* |
| | | mU |
| 105 | 26 | mN*mN*mN*mNmNNNmNmNNmNNmNNNNNm |
| | | NNNNmNNNmGUUGmUmAmGmCUCCCmUmUm |
| | | CmGmAmAmAmGmAmCmCGUUmGmCUAmCAA |
| | | U*AAGmGmCCmGmUmCmGmAmAmAmGmAmU |
| | | GUGCmCGmCAAmCGmCmUmCmUmGmCCmUm |
| | | UmCmUGGCAUCG*mU*mU |
| 101 | 27 | mN*mNNNNNNNNmNNNmNNNNNNNNNNNNm |
| | | GUUGmUmAmGmCUCCCmUmGmAmAmAmCmC |
| | | GUUmGmCUAmCAAUAAGmGmCCmGmUmCmG |
| | | mAmAmAmGmAmUGUGCmCGCmAmAmCmGmC |
| | | mUmCmUmGmCCmUmUmCmUGmGCmAmUC*m |
| | | G*mU*mU |
| 97-102 | 28 | (N)₂₀₋₂₅mGUUGmUmAmGmCUCCCmUmGm |
| | | AmAmAmCmCGUUmGmCUAmCAAUAAGmGmC |
| | | CmGmUmCmGmAmAmAmGmAmUGUGCmCGCm |
| | | AmAmCmGmCmUmCmUmGmCCmUmUmCmUGm |
| | | GCmAmUC*mG*mU*mU |
| 77 | 29 | mGUUGmUmAmGmCUCCCmUmGmAmAmAmCm |
| | | CGUUmGmCUAmCAAUAAGmGmCCmGmUmCm |
| | | GmAmAmAmGmAmUGUGCmCGCmAmAmCmGm |
| | | CmUmCmUmGmCCmUmUmCmUGmGCmAmUC* |
| | | mG*mU*mU |
| 101 | 30 | mN*mN*mN*mNmNNNmNmNNmNNmNNNNNm |
| | | NNNNmNNNmGUUGmUmAmGmCUCCCmUmGm |
| | | AmAmAmCmCGUUmGmCUAmCAAUAAGmGmC |
| | | CmGmUmCmGmAmAmAmGmAmUGUGCmCGmC |
| | | AAmCGmCmUmCmUmGmCCmUmUmCmUGGCA |
| | | UCG*mU*mU |
| 97-102 | 31 | (N)₂₀₋₂₅mGUUGmUmAmGmCUCCCmUmGm |
| | | AmAmAmCmCGUUmGmCUAmCAAUAAGmGmC |
| | | CmGmUmCmGmAmAmAmGmAmUGUGCmCGmC |
| | | AAmCGmCmUmCmUmGmCCmUmUmCmUGGCA |
| | | UCG*mU*mU |
| 77 | 32 | mGUUGmUmAmGmCUCCCmUmGmAmAmAmCm |
| | | CGUUmGmCUAmCAAUAAGmGmCCmGmUmCm |
| | | GmAmAmAmGmAmUGUGCmCGmCAAmCGmCm |
| | | UmCmUmGmCCmUmUmCmUGGCAUCG*mU*m |
| | | U |
| 97-102 | 33 | (N)₂₀₋₂₅mGUUGmUmAmGmCUCCCmUmGm |
| | | AmAmAmCmCGUUmGmCUAmCAAUAAGmGmC |
| | | CmGmUmCmGmAmAmAmGmAmUGUGCmCGCm |
| | | AmAmCmGmCmUmCmUmGmCCmUmUmCmUGm |
| | | GCmAmUC*mG*mU*mU |
| 105 | 34 | mN*mN*mN*mNmNNNmNmNNmNNmNNNNNm |
| | | NNNNmNNNmGUUGmUmAmGmCUCCCmUmUm |
| | | CmGmAmAmAmGmAmCmCGUUmGmCUAmCAA |
| | | UAAGmGmCCmGmUmCmGmAmAmAmGmAmUG |
| | | UGCmCGmCAAmCGmCmUmCmUmGmCCmUmU |
| | | mCmUGGCAUCG*mU*mU |
| 101-106 | 35 | (N)₂₀₋₂₅mGUUGmUmAmGmCUCCCmUmUm |
| | | CmGmAmAmAmGmAmCmCGUUmGmCUAmCAA |
| | | UAAGmGmCCmGmUmCmGmAmAmAmGmAmUG |
| | | UGCmCGmCAAmCGmCmUmCmUmGmCCmUmU |
| | | mCmUGGCAUCG*mU*mU |
| 81 | 36 | mGUUGmUmAmGmCUCCCmUmUmCmGmAmAm |
| | | AmGmAmCmCGUUmGmCUAmCAAUAAGmGmC |
| | | CmGmUmCmGmAmAmAmGmAmUGUGCmCGmC |
| | | AAmCGmCmUmCmUmGmCCmUmUmCmUGGCA |
| | | UCG*mU*mU |
| 101-106 | 37 | (N)₂₀₋₂₅mGUUGmUmAmGmCUCCCmUmUm |
| | | CmGmAmAmAmGmAmCmCGUUmGmCUAmCAA |
| | | U*AAGmGmCCmGmUmCmGmAmAmAmGmAmU |
| | | GUGCmCGmCAAmCGCUCUmGmCCmUmUmCm |
| | | UGGCAUCG*mU*mU |
| 81 | 38 | mGUUGmUmAmGmCUCCCmUmUmCmGmAmAm |
| | | AmGmAmCmCGUUmGmCUAmCAAU*AAGmGm |
| | | CCmGmUmCmGmAmAmAmGmAmUGUGCmCGm |
| | | CAAmCGCUCUmGmCCmUmUmCmUGGCAUCG |
| | | *mU*mU |
| 101-106 | 39 | (N)₂₀₋₂₅mGUUGmUmAmGmCUCCCmUmUm |
| | | CmGmAmAmAmGmAmCmCGUUmGmCUAmCAA |
| | | UAAGmGmCCmGmUmCmGmAmAmAmGmAmUG |
| | | UGCmCGmCAAmCGCUCUmGmCCmUmUmCmU |
| | | GGCAUCG*mU*mU |
| 81 | 40 | mGUUGmUmAmGmCUCCCmUmUmCmGmAmAm |
| | | AmGmAmCmCGUUmGmCUAmCAAUAAGmGmC |
| | | CmGmUmCmGmAmAmAmGmAmUGUGCmCGmC |
| | | AAmCGCUCUmGmCCmUmUmCmUGGCAUCG* |
| | | mU*mU |
| 101-106 | 41 | (N)₂₀₋₂₅mGUUGmUmAmGmCUCCCmUmUm |
| | | CmGmAmAmAmGmAmCmCGUUmGmCUAmCAA |
| | | U*AAGmGmCCmGmUmCmGmAmAmAmGmAmU |
| | | GUGCmCGmCAAmCGmCmUmCmUmGmCCmUm |
| | | UmCmUGGCAUCG*mU*mU |
| 81 | 42 | mGUUGmUmAmGmCUCCCmUmUmCmGmAmAm |
| | | AmGmAmCmCGUUmGmCUAmCAAU*AAGmGm |
| | | CCmGmUmCmGmAmAmAmGmAmUGUGCmCGm |
| | | CAAmCGmCmUmCmUmGmCCmUmUmCmUGGC |
| | | AUCG*mU*mU |

TABLE 2
Exemplary sgRNAs

| | SEQ | | SEQ | |
|---|---|---|---|---|
| Guide | ID | sgRNA unmodified | ID | |
| ID | NO. | sequence | NO. | sgRNA modified sequence |
| G017564 | 101 | GUGUGUCCCUCUCCCCACCCGU | 301 | mG*mUGUGUCCCmUCUmCCCCACCCGUCCmGUUGmUmAmGmCUCCCmU |
| | | CCGUUGUAGCUCCCUGGAAACC | | mGmGmAmAmAmCmCmCGUUmGmCUAmCAAUAAGmGmCCmGmUmCmG |
| | | CGUUGCUACAAUAAGGCCGUCG | | mAmAmAmGmAmUGUGCmCGCAACGCUCUmGmCCmUmUmCmUGGCAUC |
| | | AAAGAUGUGCCGCAACGCUCUG | | GUUUAmU*mU |
| | | CCUUCUGGCAUCGUUUAUU | | |
| G017565 | 102 | GGCCUGGCUGAUGAGGCCGCAC | 302 | mG*mG*mC*CUGGCUGAUGAGGCCGCACAUGUUGUAGCUCCCU*mG*mA |
| | | AUGUUGUAGCUCCCUGAAACCG | | *mA*mA*CCGUUGCUACAAUAAGGCCGUmC*mU*mG*mA*mA*mA*mA* |
| | | UUGCUACAAUAAGGCCGUCUGA | | mGAUGUGCCGCAACGCUCUGCCmU*mU*mC*mUGGCAUCGUUU*mA*m |
| | | AAAGAUGUGCCGCAACGCUCUG | | U*mC |
| | | CCUUCUGGCAUCGUUUAUC | | |
| G017566 | 103 | GGCCUGGCUGAUGAGGCCGCAC | 303 | mG*mG*mC*CUGGCUGAUGAGGCCGCACAUGUUGUAGCUCCCU*mG*mA |
| | | AUGUUGUAGCUCCCUGAAACCG | | *mA*mA*CCGUUGCUACAAUAAGGCCGUmC*mG*mA*mA*mA*mGAUGU |
| | | UUGCUACAAUAAGGCCGUCGAA | | GCCGCAACGCUCUGCCmU*mU*mC*mUGGCAUCGUUU*mA*mU*mC |
| | | AGAUGUGCCGCAACGCUCUGCC | | |
| | | UUCUGGCAUCGUUUAUC | | |
| G020031 | 104 | GUGUGUCCCUCUCCCCACCCGU | 304 | GUGUGUCCCUCUCCCCACCCGUCCGUUGUAGCUCCCUGGAAACCCGUU |
| | | CCGUUGUAGCUCCCUGGAAACC | | GCUACAAUAAGGCCGUCGAAAGAUGUGCCGCAACGCUCUGCCUUCUGG |
| | | CGUUGCUACAAUAAGGCCGUCG | | CAUCGUUUAUU |
| | | AAAGAUGUGCCGCAACGCUCUG | | |
| | | CCUUCUGGCAUCGUUUAUU | | |
| G020032 | 105 | GUGUGUCCCUCUCCCCACCCGU | 305 | mG*mU*mG*UGUCCCUCUCCCCACCCGUCCGUUGUAGCUCCCUGGAAAC |
| | | CCGUUGUAGCUCCCUGGAAACC | | CCGUUGCUACAAUAAGGCCGUCGAAAGAUGUGCCGCAACGCUCUGCCU |
| | | CGUUGCUACAAUAAGGCCGUCG | | UCUGGCAUCGUUU*mA*mU*mU |
| | | AAAGAUGUGCCGCAACGCUCUG | | |
| | | CCUUCUGGCAUCGUUUAUU | | |
| G020033 | 106 | GUGUGUCCCUCUCCCCACCCGU | 306 | mG*mU*mG*UGUCCCUCUCCCCACCCGUCCGUUGUAGCUCCCUGGAAAC |
| | | CCGUUGUAGCUCCCUGGAAACC | | CCGUUGCUACAAUAAGGCCGUCGAAAGAUGUGCCGCAACGCUCUGCCU |
| | | CGUUGCUACAAUAAGGCCGUCG | | UCUGGCAUCGUUUA*mU*mU |
| | | AAAGAUGUGCCGCAACGCUCUG | | |
| | | CCUUCUGGCAUCGUUUAUU | | |
| G020034 | 107 | GUGUGUCCCUCUCCCCACCCGU | 307 | mG*mUGUGUCCCUCUCCCCACCCGUCCGUUGUAGCUCCCUGGAAACCC |
| | | CCGUUGUAGCUCCCUGGAAACC | | GUUGCUACAAUAAGGCCGUCGAAAGAUGUGCCGCAACGCUCUGCCUUC |
| | | CGUUGCUACAAUAAGGCCGUCG | | UGGCAUCGUUUAmU*mU |
| | | AAAGAUGUGCCGCAACGCUCUG | | |
| | | CCUUCUGGCAUCGUUUAUU | | |
| G020035 | 108 | GUGUGUCCCUCUCCCCACCCGU | 308 | mG*mUGUGUCCCUCUCCCCACCCGUCCdGUUGdTdAdGdCUCCCdTdG |
| | | CCGUUGUAGCUCCCUGGAAACC | | dGdAdAdAdCdCdCGdTUdGdCUdAdCAAUAAGdGdCdCdGdUdCdGd |
| | | CGUUGCUACAAUAAGGCCGUCG | | AdAdAdGdAdUGdUGCdCGdCdAdAdCdGCUCUdGdCCdUdUdCdUGd |
| | | AAAGAUGUGCCGCAACGCUCUG | | GCdAdUCGdUdUUAmU*mU |
| | | CCUUCUGGCAUCGUUUAUU | | |
| G020036 | 109 | GUGUGUCCCUCUCCCCACCCGU | 309 | mG*mUGUGUCCCUCUCCCCACCCGUCCGUUGUAGCUCCCUGGAAACCC |
| | | CCGUUGUAGCUCCCUGGAAACC | | GUUGCUACAAUAAGGCCGUCGAAAGAUGUGCCGCAACGCUCUGCCUUC |
| | | CGUUGCUACAAUAAGGCCGUCG | | UGGCAUCGUUUAU*mU |
| | | AAAGAUGUGCCGCAACGCUCUG | | |
| | | CCUUCUGGCAUCGUUUAUU | | |
| G020037 | 110 | GUGUGUCCCUCUCCCCACCCGU | 310 | mG*mUGUGUCCCUCUCCCCACCCGUCCGUUGUAGCUCCCUGGAAACCC |
| | | CCGUUGUAGCUCCCUGGAAACC | | GUUGCUACAAUAAGGCCGUCGAAAGAUGUGCCGCAACGmCmUmCUGCC |
| | | CGUUGCUACAAUAAGGCCGUCG | | UUCUGGCAUCGUUUAmU*mU |
| | | AAAGAUGUGCCGCAACGCUCUG | | |
| | | CCUUCUGGCAUCGUUUAUU | | |
| G020038 | 111 | GUGUGUCCCUCUCCCCACCCGU | 311 | mGmUGUGUCCCUCUCCCCACCCGUCCGUUGUAGCUCCCUGGAAACCCG |
| | | CCGUUGUAGCUCCCUGGAAACC | | UUGCUACAAUAAGGCCGUCGAAAGAUGUGCCGCAACGCUCUGCCUUCU |
| | | CGUUGCUACAAUAAGGCCGUCG | | GGCAUCGUUUAmU*mU |
| | | AAAGAUGUGCCGCAACGCUCUG | | |
| | | CCUUCUGGCAUCGUUUAUU | | |
| G020039 | 112 | GUGUGUCCCUCUCCCCACCCGU | 312 | mG*mUGUGUCCCmUCUmCCCCACCCGUCCmGUUGmUmAmGmCUCCCmU |
| | | CCGUUGUAGCUCCCUGGAAACC | | mGmGmAmAmAmCmCmCGUUmGmCUAmCAAUAAGmGmCCmGmUmCmG |
| | | CGUUGCUACAAUAAGGCCGUCG | | mAmAmAmGmAmUGUGCmCGCAACGCUCUmGmCCmUmUmCmUGGCAUC |
| | | AAAGAUGUGCCGCAACGCUCUG | | GUUUAmU*mU |
| | | CCUUCUGGCAUCGUUUAUU | | |
| G020040 | 113 | GUGUGUCCCUCUCCCCACCCGU | 313 | mG*mUG*UGU*CCCmUCUmCCC*CACCCGUCCmGUUGmUmAmGmCU*C* |
| | | CCGUUGUAGCUCCCUGGAAACC | | C*C*mUmGmGmAmAmAmCmCmCGUUmGmCUAmCA*A*U*A*AG*mGmCC |
| | | CGUUGCUACAAUAAGGCCGUCG | | *mGmUmCmGmAmAmAmGmAmUGUGCmCGCAACG*C*U*C*U*mGmCCm |
| | | AAAGAUGUGCCGCAACGCUCUG | | UmUmCmUG*GCAU*C*G*UUU*AmU*mU |
| | | CCUUCUGGCAUCGUUUAUU | | |
| G020041 | 114 | GUGUGUCCCUCUCCCCACCCGU | 314 | mG*mUGUGUCCCmUCUmCCCCACCCGUCCmGUUGmUmAmGmCUCCCmU |
| | | CCGUUGUAGCUCCCUGGAAACC | | mGmGmAmAmAmCmCmCGUUmGmCUAmCAAU*AAGmGmCCmGmUmCm |
| | | CGUUGCUACAAUAAGGCCGUCG | | GmAmAmAmGmAmUGUGCmCGCAACGCUCUmGmCCmUmUmCmUGGCAU |
| | | AAAGAUGUGCCGCAACGCUCUG | | CGUUU*AmU*mU |
| | | CCUUCUGGCAUCGUUUAUU | | |
| G020042 | 115 | GUGUGUCCCUCUCCCCACCCGU | 315 | mG*mUGUGUCCCmUCUmCCCCACCCGUCCmGUUGmUmAmGmCUCCCmU |
| | | CCGUUGUAGCUCCCUGGAAACC | | mGmGmAmAmAmCmCmCGUUmGmCUAmCAAU*AAGmGmCCmGmUmCm |
| | | CGUUGCUACAAUAAGGCCGUCG | | GmAmAmAmGmAmUGUGCmCGCAACGmCmUmCUmGmCCmUmUmCmUG |
| | | AAAGAUGUGCCGCAACGCUCUG | | GCAUCGUUU*AmU*mU |
| | | CCUUCUGGCAUCGUUUAUU | | |
| G020043 | 116 | GUGUGUCCCUCUCCCCACCCGU | 316 | mG*mUGUGUCCCmUCUmCCCCACCCGUCCmGUUGmUmAmGmCUCCCmU |
| | | CCGUUGUAGCUCCCUGGAAACC | | mGmGmAmAmAmCmCmCGUUmGmCUAmCAAU*AAGmGmCCmGmUmCm |
| | | CGUUGCUACAAUAAGGCCGUCG | | GmAmAmAmGmAmUGUGCmCGCAACGCUCUmGmCCmGmAmAmAGGCA |
| | | AAAGAUGUGCCGCAACGCUCUG | | UCGUUU*AmU*mU |
| | | CCGAAAGGCAUCGUUUAUU | | |
| G020044 | 117 | GUGUGUCCCUCUCCCCACCCGU | 317 | mG*mUGUGUCCCmUCUmCCCCACCCGUCCmGUUGmUmAmGmCUCCCmU |
| | | CCGUUGUAGCUCCCUGGAAACC | | mGmGmAmAmAmCmCmCGUUmGmCUAmCAAU*AAGmGmCCmGmUmCm |
| | | CGUUGCUACAAUAAGGCCGUCG | | GmAmAmAmGmAmUGUGCmCGCAACGCUCUmGmCCmUmUmCmUGGCAU |
| | | AAAGAUGUGCCGCAACGCUCUG | | CGmU*mU |
| | | CCUUCUGGCAUCGUU | | |
| G020045 | 118 | GUGUGUCCCUCUCCCCACCCGU | 318 | GUGUGUCCCUCUCCCCACCCGUCCGUUGUAGCUCCCUGAAACCGUUGC |
| | | CCGUUGUAGCUCCCUGAAACCG | | UACAAUAAGGCCGUCGAAAGAUGUGCCGCAACGCUCUGCCUUCUGGCA |
| | | UUGCUACAAUAAGGCCGUCGAA | | UCGUUUAUU |
| | | AGAUGUGCCGCAACGCUCUGCC | | |
| | | UUCUGGCAUCGUUUAUU | | |
| G020046 | 119 | GUGUGUCCCUCUCCCCACCCGU | 319 | mG*mU*mG*UGUCCCUCUCCCCACCCGUCCGUUGUAGCUCCCUGAAACC |
| | | CCGUUGUAGCUCCCUGAAACCG | | GUUGCUACAAUAAGGCCGUCGAAAGAUGUGCCGCAACGCUCUGCCUUC |
| | | UUGCUACAAUAAGGCCGUCGAA | | UGGCAUCGUUU*mA*mU*mU |
| | | AGAUGUGCCGCAACGCUCUGCC | | |
| | | UUCUGGCAUCGUUUAUU | | |
| G020047 | 120 | GUGUGUCCCUCUCCCCACCCGU | 320 | mG*mU*mG*UGUCCCUCUCCCCACCCGUCCGUUGUAGCUCCCUGAAACC |
| | | CCGUUGUAGCUCCCUGAAACCG | | GUUGCUACAAUAAGGCCGUCGAAAGAUGUGCCGCAACGCUCUGCCUUC |
| | | UUGCUACAAUAAGGCCGUCGAA | | UGGCAUCGUUUA*mU*mU |
| | | AGAUGUGCCGCAACGCUCUGCC | | |
| | | UUCUGGCAUCGUUUAUU | | |
| G020048 | 121 | GUGUGUCCCUCUCCCCACCCGU | 321 | mG*mUGUGUCCCUCUCCCCACCCGUCCGUUGUAGCUCCCUGAAACCGU |
| | | CCGUUGUAGCUCCCUGAAACCG | | UGCUACAAUAAGGCCGUCGAAAGAUGUGCCGCAACGCUCUGCCUUCUG |
| | | UUGCUACAAUAAGGCCGUCGAA | | GCAUCGUUUAmU*mU |
| | | AGAUGUGCCGCAACGCUCUGCC | | |
| | | UUCUGGCAUCGUUUAUU | | |
| G020049 | 122 | GUGUGUCCCUCUCCCCACCCGU | 322 | mG*mUGUGUCCCUCUCCCCACCCGUCCdGUUGdTdAdGdCUCCCdTd |
| | | CCGUUGUAGCUCCCUGAAACCG | | GdAdAdAdCdCGdTUdGdCUdAdCAAUAAGdGdCdCdGdUdCdGdAd |
| | | UUGCUACAAUAAGGCCGUCGAA | | AdAdGdAdUGdUGCdCGdCdAdAdCdGCUCUdGdCCdUdUdCdUGdG |
| | | AGAUGUGCCGCAACGCUCUGCC | | CdAdUCGdUdUUAmU*mU |
| | | UUCUGGCAUCGUUUAUU | | |
| G020050 | 123 | GUGUGUCCCUCUCCCCACCCGU | 323 | mG*mUGUGUCCCUCUCCCCACCCGUCCGUUGUAGCUCCCUGAAACCGU |
| | | CCGUUGUAGCUCCCUGAAACCG | | UGCUACAAUAAGGCCGUCGAAAGAUGUGCCGCAACGCUCUGCCUUCUG |
| | | UUGCUACAAUAAGGCCGUCGAA | | GCAUCGUUUAU*mU |
| | | AGAUGUGCCGCAACGCUCUGCC | | |
| | | UUCUGGCAUCGUUUAUU | | |
| G020051 | 124 | GUGUGUCCCUCUCCCCACCCGU | 324 | mG*mUGUGUCCCUCUCCCCACCCGUCCGUUGUAGCUCCCUGAAACCGU |
| | | CCGUUGUAGCUCCCUGAAACCG | | UGCUACAAUAAGGCCGUCGAAAGAUGUGCCGCAACGmCmUmCUGCCUU |
| | | UUGCUACAAUAAGGCCGUCGAA | | CUGGCAUCGUUUAmU*mU |
| | | AGAUGUGCCGCAACGCUCUGCC | | |
| | | UUCUGGCAUCGUUUAUU | | |
| G020052 | 125 | GUGUGUCCCUCUCCCCACCCGU | 325 | mGmUGUGUCCCUCUCCCCACCCGUCCGUUGUAGCUCCCUGAAACCGUU |
| | | CCGUUGUAGCUCCCUGAAACCG | | GCUACAAUAAGGCCGUCGAAAGAUGUGCCGCAACGCUCUGCCUUCUGG |
| | | UUGCUACAAUAAGGCCGUCGAA | | CAUCGUUUAmUmU |
| | | AGAUGUGCCGCAACGCUCUGCC | | |
| | | UUCUGGCAUCGUUUAUU | | |
| G020053 | 126 | GUGUGUCCCUCUCCCCACCCGU | 326 | mG*mUGUGUCCCUCUCCCCACCCGUCCGUUGUAGCUCCCUGAAACCGU |
| | | CCGUUGUAGCUCCCUGAAACCG | | UGCUACAAUAAGGCCGUCGAAAGAUGUGCCGCAACGCUCUGCCGAAAG |
| | | UUGCUACAAUAAGGCCGUCGAA | | GCAUCGUUUAmU*mU |
| | | AGAUGUGCCGCAACGCUCUGCC | | |
| | | GAAAGGCAUCGUUUAUU | | |
| G020054 | 127 | GUGUGUCCCUCUCCCCACCCGU | 327 | mG*mUGUGUCCCUCUCCCCACCCGUCCGUUGUAGCUCCCUGAAACCGU |
| | | CCGUUGUAGCUCCCUGAAACCG | | UGCUACAAUAAGGCCGUCGAAAGAUGUGCCGCAACGCUCUGCCUUCUG |
| | | UUGCUACAAUAAGGCCGUCGAA | | GCAUCGmU*mU |
| | | AGAUGUGCCGCAACGCUCUGCC | | |
| | | UUCUGGCAUCGUU | | |
| G020055 | 128 | GUGUGUCCCUCUCCCCACCCGU | 328 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAUAAGmGmCCmGmUmCmGmAmA |
| | | UUGCUACAAUAAGGCCGUCGAA | | mAmGmAmUGUGCmCGCAACGCUCUmGmCCmUmUmCmUGGCAUCGUUU |
| | | AGAUGUGCCGCAACGCUCUGCC | | AmU*mU |
| | | UUCUGGCAUCGUUUAUU | | |
| G020056 | 129 | GUGUGUCCCUCUCCCCACCCGU | 329 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAmUAAGmGmCCmGmUmCmGmAm |
| | | UUGCUACAAUAAGGCCGUCGAA | | AmAmGmAmUGUGCmCGCAmACGCUCUmGmCCmUmUmCmUmGmGCAUC |
| | | AGAUGUGCCGCAACGCUCUGCC | | GmUmUUAmU*mU |
| | | UUCUGGCAUCGUUUAUU | | |
| G020057 | 130 | GUGUGUCCCUCUCCCCACCCGU | 330 | mG*mUmGmUmGmUCCCmUmCmUmCCCCmAmCCCGUCCmGUUGmUmAm |
| | | CCGUUGUAGCUCCCUGAAACCG | | GmCUCCCmUmGmAmAmAmCmCGUUmGmCUAmCAAUAAGmGmCCmGm |
| | | UUGCUACAAUAAGGCCGUCGAA | | UmCmGmAmAmAmGmAmUGUGCmCGCAACGCUCUmGmCCmUmUmCmU |
| | | AGAUGUGCCGCAACGCUCUGCC | | GGCAUCGUUUAmU*mU |
| | | UUCUGGCAUCGUUUAUU | | |
| G020058 | 131 | GUGUGUCCCUCUCCCCACCCGU | 331 | mG*mUmGmUmGmUCCCmUmCmUmCCCCmAmCCCGUCCmGUUGmUmAm |
| | | CCGUUGUAGCUCCCUGAAACCG | | GmCUCCCmUmGmAmAmAmCmCGUUmGmCUAmCAAmUAAGmGmCCmG |
| | | UUGCUACAAUAAGGCCGUCGAA | | mUmCmGmAmAmAmGmAmUGUGCmCGCAmACGCUCUmGmCCmUmUmC |
| | | AGAUGUGCCGCAACGCUCUGCC | | mUmGmGCAUCGmUmUUAmU*mU |
| | | UUCUGGCAUCGUUUAUU | | |
| G020059 | 132 | GUGUGUCCCUCUCCCCACCCGU | 332 | mG*mUmGmUmGmUCCCmUmCmUmCCCCmAmCCCGUCCmGUUGmUmAm |
| | | CCGUUGUAGCUCCCUGAAACCG | | GmCUCCCmUmGmAmAmAmCmCGUUmGmCUAmCAAmUAAGmGmCCmG |
| | | UUGCUACAAUAAGGCCGUCGAA | | mUmCmGmAmAmAmGmAmUGUGCmCGCAmACGCUCUmGmCCmGmAmA |
| | | AGAUGUGCCGCAACGCUCUGCC | | mAmGmGCAUCGmUmUUAmU*mU |
| | | GAAAGGCAUCGUUUAUU | | |
| G020060 | 133 | GUGUGUCCCUCUCCCCACCCGU | 333 | mG*mUmGmUmGmUCCCmUmCmUmCCCCmAmCCCGUCCmGUUGmUmAm |
| | | CCGUUGUAGCUCCCUGAAACCG | | GmCUCCCmUmGmAmAmAmCmCGUUmGmCUAmCAAmUAAGmGmCCmG |
| | | UUGCUACAAUAAGGCCGUCGAA | | mUmCmGmAmAmAmGmAmUGUGCmCGCAmACGCUCUmGmCCmUmUmC |
| | | AGAUGUGCCGCAACGCUCUGCC | | mUmGmGCAUCGmU*mU |
| | | UUCUGGCAUCGUU | | |
| G020061 | 134 | GUGUGUCCCUCUCCCCACCCGU | 334 | mG*mUG*UGU*CCCUCUCCC*CACCCGUCCGUUGUAGCU*C*C*C*U |
| | | CCGUUGUAGCUCCCUGAAACCG | | GAAACCGUUGCUACA*A*U*A*AG*GCC*GUCGAAAGAUGUGCCGCA |
| | | UUGCUACAAUAAGGCCGUCGAA | | ACG*C*U*C*U*GCCUUCUG*GCAU*C*G*UUU*AmU*mU |
| | | AGAUGUGCCGCAACGCUCUGCC | | |
| | | UUCUGGCAUCGUUUAUU | | |
| G020062 | 135 | GUGUGUCCCUCUCCCCACCCGU | 335 | mG*mUGUGUCCCUCUCCCCACCCGUCCGUUG*UAGCU*C*C*C*UGA |
| | | CCGUUGUAGCUCCCUGAAACCG | | AACCGUUGCUACA*A*UA*AG*GCC*GUCGAAAGAUG*UGCCGCAAC |
| | | UUGCUACAAUAAGGCCGUCGAA | | G*C*U*C*U*GCC*UUCUGGCAU*C*G*UUU*AmU*mU |
| | | AGAUGUGCCGCAACGCUCUGCC | | |
| | | UUCUGGCAUCGUUUAUU | | |
| G020063 | 136 | GUGUGUCCCUCUCCCCACCCGU | 336 | mG*mU*GUGUCCCUCUCC*CCACCCGUCCGUUGUAGCUCCCUGAAACCG |
| | | CCGUUGUAGCUCCCUGAAACCG | | UUGCUACAAUAAGGCCGUCGAAAGAUGUGCCGCAACGCUCUGCCUUCU |
| | | UUGCUACAAUAAGGCCGUCGAA | | GGCAUCGUUUAmU*mU |
| | | AGAUGUGCCGCAACGCUCUGCC | | |
| | | UUCUGGCAUCGUUUAUU | | |
| G020064 | 137 | GUGUGUCCCUCUCCCCACCCGU | 337 | mG*mU*GUGUCCCUCUCC*CCACCCGUCCGUUG*UAGCU*C*C*C*UGAA |
| | | CCGUUGUAGCUCCCUGAAACCG | | ACCGUUGCUACA*A*UA*AG*GCC*GUCGAAAGAUG*UGCCGCAACG*C* |
| | | UUGCUACAAUAAGGCCGUCGAA | | U*C*U*GCC*UUCUGGCAU*C*G*UUU*AmU*mU |
| | | AGAUGUGCCGCAACGCUCUGCC | | |
| | | UUCUGGCAUCGUUUAUU | | |
| G020065 | 138 | GUGUGUCCCUCUCCCCACCCGU | 338 | mG*mUGUGUCCCUCUCCC*CACCCGUCCGUUGUAGCUCCCUGAAACCGU |
| | | CCGUUGUAGCUCCCUGAAACCG | | UGCUACAAU*AAGGCCGUCGAAAGAUGUGCCGCAACGCUCUGCCUUCU |
| | | UUGCUACAAUAAGGCCGUCGAA | | GGCAUCGUUU*AmU*mU |
| | | AGAUGUGCCGCAACGCUCUGCC | | |
| | | UUCUGGCAUCGUUUAUU | | |
| G020066 | 139 | GUGUGUCCCUCUCCCCACCCGU | 339 | mG*mUGUGUCCCUCUCCC*CACCCGUCCGUUGUAGCUCCCUGAAACCGU |
| | | CCGUUGUAGCUCCCUGAAACCG | | UGCUACAAU*AAGGCCGUCGAAAGAUGUGCCGCAACGCUCUGCCGAAA |
| | | UUGCUACAAUAAGGCCGUCGAA | | GGCAUCGUUU*AmU*mU |
| | | AGAUGUGCCGCAACGCUCUGCC | | |
| | | GAAAGGCAUCGUUUAUU | | |
| G020067 | 140 | GUGUGUCCCUCUCCCCACCCGU | 340 | mG*mUGUGUCCCUCUCCC*CACCCGUCCGUUGUAGCUCCCUGAAACCGU |
| | | CCGUUGUAGCUCCCUGAAACCG | | UGCUACAAU*AAGGCCGUCGAAAGAUGUGCCGCAACGCUCUGCCUUCU |
| | | UUGCUACAAUAAGGCCGUCGAA | | GGCAUCGmU*mU |
| | | AGAUGUGCCGCAACGCUCUGCC | | |
| | | UUCUGGCAUCGUU | | |
| G020068 | 141 | GUGUGUCCCUCUCCCCACCCGU | 341 | mG*mUG*UGU*CCCmUCUmCCC*CACCCGUCCmGUUGmUmAmGmCU*C* |
| | | CCGUUGUAGCUCCCUGAAACCG | | C*C*mUmGmAmAmAmCmCGUUmGmCUAmCA*A*U*A*AG*mGmCC*mGm |
| | | UUGCUACAAUAAGGCCGUCGAA | | UmCmGmAmAmAmGmAmUGUGCmCGCAACG*C*U*C*U*mGmCCmUmUm |
| | | AGAUGUGCCGCAACGCUCUGCC | | CmUG*GCAU*C*G*UUU*AmU*mU |
| | | UUCUGGCAUCGUUUAUU | | |
| G020069 | 142 | GUGUGUCCCUCUCCCCACCCGU | 342 | mG*mUGUGUCCCmUCUmCCCCACCCGUCCmGUUG*mUmAmGmCU*C*C* |
| | | CCGUUGUAGCUCCCUGAAACCG | | C*mUmGmAmAmAmCmCGUUmGmCUAmCA*A*mUA*AG*mGmCC*mGmU |
| | | UUGCUACAAUAAGGCCGUCGAA | | mCmGmAmAmAmGmAmUG*UGCmCGCAmACG*C*U*C*U*mGmCCmUmU |
| | | AGAUGUGCCGCAACGCUCUGCC | | mCmUmGmGCAU*C*G*mUmUU*AmU*mU |
| | | UUCUGGCAUCGUUUAUU | | |
| G020070 | 143 | GUGUGUCCCUCUCCCCACCCGU | 343 | mG*mUmGmUmGmUCCCmUmCmUmCCCCmAmCCCGUCCmGUUG*mUmA |
| | | CCGUUGUAGCUCCCUGAAACCG | | mGmCU*C*C*C*mUmGmAmAmAmCmCGUUmGmCUAmCA*A*mUA*AG*m |
| | | UUGCUACAAUAAGGCCGUCGAA | | GmCC*mGmUmCmGmAmAmAmGmAmUG*UGCmCGCAmACG*C*U*C*U*m |
| | | AGAUGUGCCGCAACGCUCUGCC | | GmCCmUmUmCmUmGmGCAU*C*G*mUmUU*AmU*mU |
| | | UUCUGGCAUCGUUUAUU | | |
| G020071 | 144 | GUGUGUCCCUCUCCCCACCCGU | 344 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmUmUmCmUGGCAUCGUU |
| | | AGAUGUGCCGCAACGCUCUGCC | | U*AmU*mU |
| | | UUCUGGCAUCGUUUAUU | | |
| G020072 | 145 | GUGUGUCCCUCUCCCCACCCGU | 345 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmGmAmAmAGGCAUCGUU |
| | | AGAUGUGCCGCAACGCUCUGCC | | U*AmU*mU |
| | | GAAAGGCAUCGUUUAUU | | |
| G020073 | 146 | GUGUGUCCCUCUCCCCACCCGU | 346 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmUmUmCmUGGCAUCGmU |
| | | AGAUGUGCCGCAACGCUCUGCC | | *mU |
| | | UUCUGGCAUCGUU | | |
| G020361 | 147 | GGCCUGGCUGAUGAGGCCGCAC | 347 | mG*mGC*CUGGCUmGAUmGAGGCCGCACAUmGUUGmUmAmGmCUCCCU |
| | | AUGUUGUAGCUCCCUCGAAAGC | | mCmGmAmAmAmGCCGUUmGmCUAmCAAU*A*AGmGmCCmGmUmCmGm |
| | | CGUUGCUACAAUAAGGCCGUCG | | AmAmAmGmAmUGUGCmCGCAACGCUCUmGmCCmUmUmCmUGGCAUCG |
| | | AAAGAUGUGCCGCAACGCUCUG | | UUU*AmU*mU |
| | | CCUUCUGGCAUCGUUUAUU | | |
| G020711 | 148 | GUGUGUCCCUCUCCCCACCCGU | 348 | GUGUGUCCCUCUCCCCACCCGUCCGUUGUAGCUCCCUGAAACCGUUGC |
| | | CCGUUGUAGCUCCCUGAAACCG | | UACAAUAAGGCCGUCGAAAGAUGUGCCGCAACGCUCUGCCUUCUGGCA |
| | | UUGCUACAAUAAGGCCGUCGAA | | UCGUU |
| | | AGAUGUGCCGCAACGCUCUGCC | | |
| | | UUCUGGCAUCGUU | | |
| G020712 | 149 | GUGUGUCCCUCUCCCCACCCGU | 349 | GUGUGUCCCmUCUmCCCCACCCGUCCmGUUGmUmAmGmCUCCCmUmG |
| | | CCGUUGUAGCUCCCUGAAACCG | | mAmAmAmCmCGUUmGmCUAmCAAU*AAGmGmCCmGmUmCmGmAmAm |
| | | UUGCUACAAUAAGGCCGUCGAA | | AmGmAmUGUGCmCGCAACGCUCUmGmCCmUmUmCmUGGCAUCGUU |
| | | AGAUGUGCCGCAACGCUCUGCC | | |
| | | UUCUGGCAUCGUU | | |
| G020713 | 150 | GUGUGUCCCUCUCCCCACCCGU | 350 | G*UGUGUCCCUCUCCCCACCCGUCCGUUGUAGCUCCCUGAAACCGUUGC |
| | | CCGUUGUAGCUCCCUGAAACCG | | UACAAUAAGGCCGUCGAAAGAUGUGCCGCAACGCUCUGCCUUCUGGCA |
| | | UUGCUACAAUAAGGCCGUCGAA | | UCGU*U |
| | | AGAUGUGCCGCAACGCUCUGCC | | |
| | | UUCUGGCAUCGUU | | |
| G020714 | 151 | GUGUGUCCCUCUCCCCACCCGU | 351 | mGmUGUGUCCCUCUCCCCACCCGUCCGUUGUAGCUCCCUGAAACCGUU |
| | | CCGUUGUAGCUCCCUGAAACCG | | GCUACAAUAAGGCCGUCGAAAGAUGUGCCGCAACGCUCUGCCUUCUGG |
| | | UUGCUACAAUAAGGCCGUCGAA | | CAUCGmUmU |
| | | AGAUGUGCCGCAACGCUCUGCC | | |
| | | UUCUGGCAUCGUU | | |
| G020715 | 152 | GUGUGUCCCUCUCCCCACCCGU | 352 | mGmUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAUAAGmGmCCmGmUmCmGmAmA |
| | | UUGCUACAAUAAGGCCGUCGAA | | mAmGmAmUGUGCmCGCAACGCUCUmGmCCmUmUmCmUGGCAUCGmUm |
| | | AGAUGUGCCGCAACGCUCUGCC | | U |
| | | UUCUGGCAUCGUU | | |
| G020716 | 153 | GUGUGUCCCUCUCCCCACCCGU | 353 | mGmUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAUAAGmGmCCmGmUmCmGmAmA |
| | | UUGCUACAAUAAGGCCGUCGAA | | mAmGmAmUGUGCmCGCAACGCUCUmGmCCmUmUmCmUGGCAUCGmU* |
| | | AGAUGUGCCGCAACGCUCUGCC | | mU |
| | | UUCUGGCAUCGUU | | |
| G020717 | 154 | GUGUGUCCCUCUCCCCACCCGU | 354 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAUAAGmGmCCmGmUmCmGmAmA |
| | | UUGCUACAAUAAGGCCGUCGAA | | mAmGmAmUGUGCmCGCAACGCUCUmGmCCmUmUmCmUGGCAUCGmUm |
| | | AGAUGUGCCGCAACGCUCUGCC | | U |
| | | UUCUGGCAUCGUU | | |
| G020718 | 155 | GUGUGUCCCUCUCCCCACCCGU | 355 | mG*mU*mG*UGUCCCmUCUmCCCCACCCGUCCmGUUGmUmAmGmCUCC |
| | | CCGUUGUAGCUCCCUGAAACCG | | CmUmGmAmAmAmCmCGUUmGmCUAmCAAU*AAGmGmCCmGmUmCmG |
| | | UUGCUACAAUAAGGCCGUCGAA | | mAmAmAmGmAmUGUGCmCGCAACGCUCUmGmCCmUmUmCmUGGCAUC |
| | | AGAUGUGCCGCAACGCUCUGCC | | GmU*mU |
| | | UUCUGGCAUCGUU | | |
| G020719 | 156 | GUGUGUCCCUCUCCCCACCCGU | 356 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmUmUmCmUGGCAUC*mG |
| | | AGAUGUGCCGCAACGCUCUGCC | | *mU*mU |
| | | UUCUGGCAUCGUU | | |
| G020720 | 157 | GUGUGUCCCUCUCCCCACCCGU | 357 | mG*mU*mG*UGUCCCmUCUmCCCCACCCGUCCmGUUGmUmAmGmCUCC |
| | | CCGUUGUAGCUCCCUGAAACCG | | CmUmGmAmAmAmCmCGUUmGmCUAmCAAU*AAGmGmCCmGmUmCmG |
| | | UUGCUACAAUAAGGCCGUCGAA | | mAmAmAmGmAmUGUGCmCGCAACGCUCUmGmCCmUmUmCmUGGCAUC |
| | | AGAUGUGCCGCAACGCUCUGCC | | *mG*mU*mU |
| | | UUCUGGCAUCGUU | | |
| G020721 | 158 | GUGUGUCCCUCUCCCCACCCGU | 358 | mG*mU*GUGUCCCmUCUmCCCCACCCGUCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCAACGCUCUmGmCCmUmUmCmUGGCAUCG* |
| | | AGAUGUGCCGCAACGCUCUGCC | | mU*mU |
| | | UUCUGGCAUCGUU | | |
| G020722 | 159 | GUGUGUCCCUCUCCCCACCCGU | 359 | mG*UGUGUCCCmUCUmCCCCACCCGUCCmGUUGmUmAmGmCUCCCmUm |
| | | CCGUUGUAGCUCCCUGAAACCG | | GmAmAmAmCmCGUUmGmCUAmCAAU*AAGmGmCCmGmUmCmGmAmA |
| | | UUGCUACAAUAAGGCCGUCGAA | | mAmGmAmUGUGCmCGCAACGCUCUmGmCCmUmUmCmUGGCAUCGU*m |
| | | AGAUGUGCCGCAACGCUCUGCC | | U |
| | | UUCUGGCAUCGUU | | |
| G020723 | 160 | GUGUGUCCCUCUCCCCACCCGU | 360 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmGmAmAmAGGCAUCGmU |
| | | AGAUGUGCCGCAACGCUCUGCC | | *mU |
| | | GAAAGGCAUCGUU | | |
| G020724 | 161 | GUGUGUCCCUCUCCCCACCCGU | 361 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGmCmUmCUmGmCCmUmUmCmUGGCAUC |
| | | AGAUGUGCCGCAACGCUCUGCC | | GmU*mU |
| | | UUCUGGCAUCGUU | | |
| G020725 | 162 | GUGUGUCCCUCUCCCCACCCGU | 362 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGC*U*C*UmGmCCmUmUmCmUGGCAUCG |
| | | AGAUGUGCCGCAACGCUCUGCC | | mU*mU |
| | | UUCUGGCAUCGUU | | |
| G020726 | 163 | GUGUGUCCCUCUCCCCACCCGU | 363 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAUAAGmGmCCmGmUmCmGmAmA |
| | | UUGCUACAAUAAGGCCGUCGAA | | mAmGmAmUGUGCmCGCAACGCUCUmGmCCmUmUmCmUGGCAUCGmU* |
| | | AGAUGUGCCGCAACGCUCUGCC | | mU |
| | | UUCUGGCAUCGUU | | |
| G020727 | 164 | GUGUGUCCCUCUCCCCACCCGU | 364 | mG*mUGUGUCCCUCUCCCCACCCGUCCGUUGUAGCUCCCUGAAACCGU |
| | | CCGUUGUAGCUCCCUGAAACCG | | UGCUACAAU*AAGGCCGUCGAAAGAUGUGCCGCAACGCUCUGCCUUCU |
| | | UUGCUACAAUAAGGCCGUCGAA | | GGCAUCGmU*mU |
| | | AGAUGUGCCGCAACGCUCUGCC | | |
| | | UUCUGGCAUCGUU | | |
| G020728 | 165 | GUGUGUCCCUCUCCCCACCCGU | 365 | mG*mU*GUGUCCCmUCUmCC*CCACCCGUCCmGUUGmUmAmGmCUCCC |
| | | CCGUUGUAGCUCCCUGAAACCG | | mUmGmAmAmAmCmCGUUmGmCUAmCAAU*AAGmGmCCmGmUmCmGm |
| | | UUGCUACAAUAAGGCCGUCGAA | | AmAmAmGmAmUGUGCmCGCAACGCUCUmGmCCmUmUmCmUGGCAUCG |
| | | AGAUGUGCCGCAACGCUCUGCC | | mU*mU |
| | | UUCUGGCAUCGUU | | |
| G020729 | 166 | GUGUGUCCCUCUCCCCACCCGU | 366 | mG*mUGUGUCCCmUCUmCCCCACCCGUCCmGUUG*mUmAmGmCU*C*C* |
| | | CCGUUGUAGCUCCCUGAAACCG | | C*mUmGmAmAmAmCmCGUUmGmCUAmCA*A*UA*AG*mGmCC*mGmUm |
| | | UUGCUACAAUAAGGCCGUCGAA | | CmGmAmAmAmGmAmUG*UGCmCGCAACG*C*U*C*U*mGmCC*mUmUmC |
| | | AGAUGUGCCGCAACGCUCUGCC | | mUGGCAU*C*G*mU*mU |
| | | UUCUGGCAUCGUU | | |
| G020730 | 167 | GUGUGUCCCUCUCCCCACCCGU | 367 | mG*mUGUGUCCCmUCUmCCCCACCCGUCCmGUUG*mUmAmGmCU*C*C* |
| | | CCGUUGUAGCUCCCUGAAACCG | | C*mUmGmAmAmAmCmCGUUmGmCUAmCA*A*U*A*AG*mGmCC*mGmU |
| | | UUGCUACAAUAAGGCCGUCGAA | | mCmGmAmAmAmGmAmUG*UGCmCGCAACG*C*U*C*U*mGmCC*mUmUm |
| | | AGAUGUGCCGCAACGCUCUGCC | | CmUGGCAU*C*G*mU*mU |
| | | UUCUGGCAUCGUU | | |
| G020731 | 168 | GUGUGUCCCUCUCCCCACCCGU | 368 | mG*mU*GUGUCCCmUCUmCC*CCACCCGUCCmGUUG*mUmAmGmCU*C* |
| | | CCGUUGUAGCUCCCUGAAACCG | | C*C*mUmGmAmAmAmCmCGUUmGmCUAmCA*A*UA*AG*mGmCC*mGm |
| | | UUGCUACAAUAAGGCCGUCGAA | | UmCmGmAmAmAmGmAmUG*UGCmCGCAACG*C*U*C*U*mGmCC*mUmU |
| | | AGAUGUGCCGCAACGCUCUGCC | | mCmUGGCAU*C*G*mU*mU |
| | | UUCUGGCAUCGUU | | |
| G020732 | 169 | GUGUGUCCCUCUCCCCACCCGU | 369 | mG*mU*GUGUCCCmUCUmCC*CCACCCGUCCmGUUG*mUmAmGmCU*C* |
| | | CCGUUGUAGCUCCCUGAAACCG | | C*C*mUmGmAmAmAmCmCGUUmGmCUAmCA*A*U*A*AG*mGmCC*mGm |
| | | UUGCUACAAUAAGGCCGUCGAA | | UmCmGmAmAmAmGmAmUG*UGCmCGCAACG*C*U*C*U*mGmCC*mUmU |
| | | AGAUGUGCCGCAACGCUCUGCC | | mCmUGGCAU*C*G*mU*mU |
| | | UUCUGGCAUCGUU | | |
| G020733 | 170 | GUGUGUCCCUCUCCCCACCCGU | 370 | mG*mUG*UGU*CCCmUCUmCCC*CACCCGUCCmGUUGmUmAmGmCUCCC |
| | | CCGUUGUAGCUCCCUGAAACCG | | mUmGmAmAmAmCmCGUUmGmCUAmCAAU*AAGmGmCCmGmUmCmGm |
| | | UUGCUACAAUAAGGCCGUCGAA | | AmAmAmGmAmUGUGCmCGCAACGCUCUmGmCCmUmUmCmUGGCAUCG |
| | | AGAUGUGCCGCAACGCUCUGCC | | mU*mU |
| | | UUCUGGCAUCGUU | | |
| G020734 | 171 | GUGUGUCCCUCUCCCCACCCGU | 371 | mG*mUGUGUCCCmUCUmCCCCACCCGUCCmGUUGmUmAmGmCU*C*C*C |
| | | CCGUUGUAGCUCCCUGAAACCG | | *mUmGmAmAmAmCmCGUUmGmCUAmCA*A*U*A*AG*mGmCC*mGmUm |
| | | UUGCUACAAUAAGGCCGUCGAA | | CmGmAmAmAmGmAmUGUGCmCGCAACG*C*U*C*U*mGmCCmUmUmCm |
| | | AGAUGUGCCGCAACGCUCUGCC | | UG*GCAU*C*G*mU*mU |
| | | UUCUGGCAUCGUU | | |
| G020735 | 172 | GUGUGUCCCUCUCCCCACCCGU | 372 | mG*mUG*UGU*CCCmUCUmCCC*CACCCGUCCmGUUGmUmAmGmCU*C* |
| | | CCGUUGUAGCUCCCUGAAACCG | | C*C*mUmGmAmAmAmCmCGUUmGmCUAmCA*A*U*A*AG*mGmCC*mGm |
| | | UUGCUACAAUAAGGCCGUCGAA | | UmCmGmAmAmAmGmAmUGUGCmCGCAACG*C*U*C*U*mGmCCmUmUm |
| | | AGAUGUGCCGCAACGCUCUGCC | | CmUG*GCAU*C*G*mU*mU |
| | | UUCUGGCAUCGUU | | |
| G020736 | 173 | GUGUGUCCCUCUCCCCACCCGU | 373 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmUmUmCmUGGCAUCGmU |
| | | AGAUGUGCCGCAACGCUCUGCC | | *mUmU |
| | | UUCUGGCAUCGUUU | | |
| G020737 | 174 | GUGUGUCCCUCUCCCCACCCGU | 374 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmUmUmCmUGGCAUCGmU |
| | | AGAUGUGCCGCAACGCUCUGCC | | *mUmU*mA |
| | | UUCUGGCAUCGUUUA | | |
| G020738 | 175 | GUGUGUCCCUCUCCCCACCCGU | 375 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCmUmUmCmUGGCAUCGmU |
| | | AGAUGUGCCGCAACGCUCUGCC | | *mUmU*mA*mU |
| | | UUCUGGCAUCGUUUAU | | |
| G020739 | 176 | GUGUGUCCCUCUCCCCACCCGU | 376 | mG*mUGUGUCCCmUCUmCCCCACCCGUCCmGUUGmUmAmGmCUCCCmU |
| | | CCGUUGUAGCUCCCUUGGAAAC | | mUmGmGmAmAmAmCmCmAmCGUUmGmCUAmCAAU*AAGmGmCCmGm |
| | | CACGUUGCUACAAUAAGGCCGU | | UmCmGmAmAmAmGmAmUGUGCmCGCAACGCUCUmGmCCmUmUmCmU |
| | | CGAAAGAUGUGCCGCAACGCUC | | GGCAUCGmU*mU |
| | | UGCCUUCUGGCAUCGUU | | |
| G020740 | 177 | GUGUGUCCCUCUCCCCACCCGU | 377 | mG*mUGUGUCCCmUCUmCCCCACCCGUCCmGUUGmUmAmGmCUCCCmG |
| | | CCGUUGUAGCUCCCGAAACGUU | | mAmAmAmCGUUmGmCUAmCAAU*AAGmGmCCmGmUmCmGmAmAmAm |
| | | GCUACAAUAAGGCCGUCGAAAG | | GmAmUGUGCmCGCAACGCUCUmGmCCmUmUmCmUGGCAUCGmU*mU |
| | | AUGUGCCGCAACGCUCUGCCUU | | |
| | | CUGGCAUCGUU | | |
| G020741 | 178 | GUGUGUCCCUCUCCCCACCCGU | 378 | mG*mUGUGUCCCmUCUmCCCCACCCGUCCmGUUGmUmAmGmCUCCmG |
| | | CCGUUGUAGCUCCGAAACUUGC | | mAmAmAmCUUmGmCUAmCAAU*AAGmGmCCmGmUmCmGmAmAmAmG |
| | | UACAAUAAGGCCGUCGAAAGAU | | mAmUGUGCmCGCAACGCUCUmGmCCmUmUmCmUGGCAUCGmU*mU |
| | | GUGCCGCAACGCUCUGCCUUCU | | |
| | | GGCAUCGUU | | |
| G020742 | 179 | GUGUGUCCCUCUCCCCACCCGU | 379 | mG*mUGUGUCCCmUCUmCC*CCACCCGUCCmGUUG*mUmAmGmCU*C*C |
| | | CCGUUGUAGCUCCCUGAAACCG | | *C*mUmGmAmAmAmCmCGUUmGmCUAmCA*A*U*A*AG*mGmCC*mGmU |
| | | UUGCUACAAUAAGGCCGUCGAA | | mCmGmAmAmAmGmAmUG*UGCmCGCAACG*mCmUmCU*mGmCC*mUm |
| | | AGAUGUGCCGCAACGCUCUGCC | | UmCmUGGCAU*C*G*mU*mU |
| | | UUCUGGCAUCGUU | | |
| G020743 | 180 | GUGUGUCCCUCUCCCCACCCGU | 380 | mG*mU*mG*UGUCCCmUCUmCC*CCACCCGUCCmGUUG*mUmAmGmCU* |
| | | CCGUUGUAGCUCCCUGAAACCG | | C*C*C*mUmGmAmAmAmCmCGUUmGmCUAmCA*A*U*A*AG*mGmCC* |
| | | UUGCUACAAUAAGGCCGUCGAA | | mGmUmCmGmAmAmAmGmAmUG*UGCmCGCAACG*mCmUmCU*mGmCC* |
| | | AGAUGUGCCGCAACGCUCUGCC | | mUmUmCmUGGCAU*C*G*mU*mU |
| | | UUCUGGCAUCGUU | | |
| G020744 | 181 | GUGUGUCCCUCUCCCCACCCGU | 381 | mG*mUGUGUCCCmUCUmCC*CCACCCGUCCmGUUG*mUmAmGmCU*C*C |
| | | CCGUUGUAGCUCCCUGAAACCG | | *C*mUmGmAmAmAmCmCGUUmGmCUAmCA*A*U*A*AG*mGmCC*mGmU |
| | | UUGCUACAAUAAGGCCGUCGAA | | mCmGmAmAmAmGmAmUG*UGCmCGCAACG*mCmUmCU*mGmCC*mGm |
| | | AGAUGUGCCGCAACGCUCUGCC | | AmAmAGGCAU*C*G*mU*mU |
| | | GAAAGGCAUCGUU | | |
| G020745 | 182 | GUGUGUCCCUCUCCCCACCCGU | 382 | mG*mU*mG*UGUCCCmUCUmCC*CCACCCGUCCmGUUG*mUmAmGmCU* |
| | | CCGUUGUAGCUCCCUGAAACCG | | C*C*C*mUmGmAmAmAmCmCGUUmGmCUAmCA*A*U*A*AG*mGmCC* |
| | | UUGCUACAAUAAGGCCGUCGAA | | mGmUmCmGmAmAmAmGmAmUG*UGCmCGCAACG*mCmUmCU*mGmCC* |
| | | AGAUGUGCCGCAACGCUCUGCC | | mGmAmAmAGGCAU*C*G*mU*mU |
| | | GAAAGGCAUCGUU | | |
| G020746 | 183 | GUGUGUCCCUCUCCCCACCCGU | 383 | mG*mUmGmUmGUCmCmCmUmCUmCCCCAmCCCGmUCCmGUUGmUmAm |
| | | CCGUUGUAGCUCCCUGAAACCG | | GmCUCCCmUmGmAmAmAmCmCGUUmGmCUAmCAAU*AAGmGmCCmGm |
| | | UUGCUACAAUAAGGCCGUCGAA | | UmCmGmAmAmAmGmAmUGUGCmCGCAACGCUCUmGmCCmUmUmCmU |
| | | AGAUGUGCCGCAACGCUCUGCC | | GGCAUCGmU*mU |
| | | UUCUGGCAUCGUU | | |
| G020747 | 184 | GUGUGUCCCUCUCCCCACCCGU | 384 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGmUUmGmCUmAmCAAU*AAGmGmCCmGmUmCmGm |
| | | UUGCUACAAUAAGGCCGUCGAA | | AmAmAmGmAmUGUGCmCGCAACGCUCUmGmCCmUmUmCmUGGCAUCG |
| | | AGAUGUGCCGCAACGCUCUGCC | | mU*mU |
| | | UUCUGGCAUCGUU | | |
| G020748 | 185 | GUGUGUCCCUCUCCCCACCCGU | 385 | mG*mUGUGUCCCmUCUmCCCCACCCGUCCmGUUGmUmAmGmCUCCCm |
| | | CCGUUGUAGCUCCCUGAAACCG | | UmGmAmAmAmCmCGUUmGmCUAmCAAU*AAGmGmCmCmGmUmCmGmA |
| | | UUGCUACAAUAAGGCCGUCGAA | | mAmAmGmAmUGmUGCmCGmCAACGCUCUmGmCCmUmUmCmUGGCAUC |
| | | AGAUGUGCCGCAACGCUCUGCC | | GmU*mU |
| | | UUCUGGCAUCGUU | | |
| G020749 | 186 | GUGUGUCCCUCUCCCCACCCGU | 386 | mG*mUGUGUCCCmUCUmCCCCACCCGUCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCmAmAmCmGCUCUmGmCCmUmUmCmUGmGC |
| | | AGAUGUGCCGCAACGCUCUGCC | | mAmUCGmU*mU |
| | | UUCUGGCAUCGUU | | |
| G020750 | 187 | GUGUGUCCCUCUCCCCACCCGU | 387 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGmUUmGmCUmAmCAAU*AAGmGmCmCmGmUmCmG |
| | | UUGCUACAAUAAGGCCGUCGAA | | mAmAmAmGmAmUGmUGCmCGmCmAmAmCmGCUCUmGmCCmUmUmCm |
| | | AGAUGUGCCGCAACGCUCUGCC | | UGmGCmAmUCGmU*mU |
| | | UUCUGGCAUCGUU | | |
| G020751 | 188 | GUGUGUCCCUCUCCCCACCCGU | 388 | mG*mUGUGUCCCmUCUmCCCCACCCGUCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmUmGm |
| | | UUGCUACAAUAAGGCCGUCUGA | | AmAmAmAmGmAmUGUGCmCGCAACGCUCUmGmCCmUmUmCmUGGCAU |
| | | AAAGAUGUGCCGCAACGCUCUG | | CGmU*mU |
| | | CCUUCUGGCAUCGUU | | |
| G020752 | 189 | GUGUGUCCCUCUCCCCACCCGU | 389 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCAACGCUCUmGmCCCmUmUmCmUmGGGCAUC |
| | | AGAUGUGCCGCAACGCUCUGCC | | GmU*mU |
| | | CUUCUGGGCAUCGUU | | |
| G020753 | 190 | GUGUGUCCCUCUCCCCACCCGU | 390 | mG*mUGUGUCCCmUCUmCCCCACCCGUCCmGUUGmUmAmGmCUCCCmU |
| | | CCGUUGUAGCUCCCUGAAACCG | | mGmAmAmAmCmCGUUmGmCUAmCAAU*AAGmGmCCmGmUmCmUmGm |
| | | UUGCUACAAUAAGGCCGUCUGA | | AmAmAmAmGmAmUGUGCmCGCAACGCUCUmGmCCCmUmUmCmUmGG |
| | | AAAGAUGUGCCGCAACGCUCUG | | GCAUCGmU*mU |
| | | CCCUUCUGGGCAUCGUU | | |
| G020754 | 191 | GUGUGUCCCUCUCCCCACCCGU | 391 | mG*mU*mG*UGUCCCmUCUmCCCCACCCGUCCmGUUGmUmAmGmCUCC |
| | | CCGUUGUAGCUCCCUGAAACCG | | CmUmGmAmAmAmCmCGUUmGmCUAmCAAU*AAGmGmCCmGmUmCmU |
| | | UUGCUACAAUAAGGCCGUCUGA | | mGmAmAmAmAmGmAmUGUGCmCGCAACGCUCUmGmCCmUmUmCmUG |
| | | AAAGAUGUGCCGCAACGCUCUG | | GCAUCGmU*mU |
| | | CCUUCUGGCAUCGUU | | |
| G020755 | 192 | GUGUGUCCCUCUCCCCACCCGU | 392 | mG*mU*mG*UGUCCCmUCUmCCCCACCCGUCCmGUUGmUmAmGmCUCC |
| | | CCGUUGUAGCUCCCUGAAACCG | | CmUmGmAmAmAmCmCGUUmGmCUAmCAAU*AAGmGmCCmGmUmCmG |
| | | UUGCUACAAUAAGGCCGUCGAA | | mAmAmAmGmAmUGUGCmCGCAACGCUCUmGmCCCmUmUmCmUmGGGC |
| | | AGAUGUGCCGCAACGCUCUGCC | | AUCGmU*mU |
| | | CUUCUGGGCAUCGUU | | |
| G020756 | 193 | GUGUGUCCCUCUCCCCACCCGU | 393 | mG*mU*mG*UGUCCCmUCUmCCCCACCCGUCCmGUUGmUmAmGmCUCC |
| | | CCGUUGUAGCUCCCUGAAACCG | | CmUmGmAmAmAmCmCGUUmGmCUAmCAAU*AAGmGmCCmGmUmCmU |
| | | UUGCUACAAUAAGGCCGUCUGA | | mGmAmAmAmAmGmAmUGUGCmCGCAACGCUCUmGmCCCmUmUmCmU |
| | | AAAGAUGUGCCGCAACGCUCUG | | mGGGCAUCGmU*mU |
| | | CCCUUCUGGGCAUCGUU | | |
| G020848 | 194 | UGAGGACCGCCCUGGGCCUGGG | 394 | mU*mGA*GGACCGmCCCmUGGGCCUGGGAGmGUUGmUmAmGmCUCCCU |
| | | AGGUUGUAGCUCCCUCGAAAGC | | mCmGmAmAmAmGCCGUUmGmCUAmCAAU*A*AGmGmCCmGmUmCmGm |
| | | CGUUGCUACAAUAAGGCCGUCG | | AmAmAmGmAmUGUGCmCGCAACGCUCUmGmCCmUmUmCmUGGCAUCG |
| | | AAAGAUGUGCCGCAACGCUCUG | | UUU*AmU*mU |
| | | CCUUCUGGCAUCGUUUAUU | | |
| G021237 | 195 | GGGGCCAGCUUCAGACACAAAU | 395 | mG*mGGGCCAGCmUUCmAGACACAAAUACmGUUGmUmAmGmCUCCCm |
| | | ACGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021238 | 196 | UUUUCACAGCCAACGACUCUGG | 396 | mU*mUUUCACAGmCCAmACGACUCUGGCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021239 | 197 | CAUCAGAGGACAUUUGGAUUCU | 397 | mC*mAUCAGAGGmACAmUUUGGAUUCUCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021240 | 198 | CAUGGAACGGGGAAAUGCCAAG | 398 | mC*mAUGGAACGmGGGmAAAUGCCAAGUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021241 | 199 | AAAUCGUACUGGAAGACACUUG | 399 | mA*mAAUCGUACmUGGmAAGACACUUGGCmGUUGmUmAmGmCUCCCm |
| | | GCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021242 | 200 | AAUCGUACUGGAAGACACUUGG | 400 | mA*mAUCGUACUmGGAmAGACACUUGGCAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021243 | 201 | UCUGGCCAUCGCCACUACACCA | 401 | mU*mCUGGCCAUmCGCmCACUACACCAUCmGUUGmUmAmGmCUCCCm |
| | | UCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021244 | 202 | CUGGCCAUCGCCACUACACCAU | 402 | mC*mUGGCCAUCmGCCmACUACACCAUCGmGUUGmUmAmGmCUCCCm |
| | | CGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021245 | 203 | ACCAUCGCAGCCCUGCUCAGCC | 403 | mA*mCCAUCGCAmGCCmCUGCUCAGCCCAmGUUGmUmAmGmCUCCCmU |
| | | CAGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCmAmAmCmGCUCUmGmCCmUmUmCmUGmGC |
| | | AGAUGUGCCGCAACGCUCUGCC | | mAmUC*mG*mU*mU |
| | | UUCUGGCAUCGUU | | |
| G021246 | 204 | UGGACUGGUAUUUGUGUCUGAA | 404 | mU*mGGACUGGUmAUUmUGUGUCUGAAGCmGUUGmUmAmGmCUCCCm |
| | | GCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021247 | 205 | CUGCCUCGGACAGCAUCCAGGA | 405 | mC*mUGCCUCGGmACAmGCAUCCAGGACUmGUUGmUmAmGmCUCCCm |
| | | CUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021248 | 206 | CUACAAGCUUACCCAGAGGCAA | 406 | mC*mUACAAGCUmUACmCCAGAGGCAAAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021249 | 207 | CAGAAUUGAGAGACUCAGCCCA | 407 | mC*mAGAAUUGAmGAGmACUCAGCCCAGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021250 | 208 | GAUCCACAAGCUCCUGACAGGA | 408 | mG*mAUCCACAAmGCUmCCUGACAGGAUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021251 | 209 | AUCCACAAGCUCCUGACAGGAU | 409 | mA*mUCCACAAGmCUCmCUGACAGGAUGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021252 | 210 | GGACUGGUAUUUGUGUCUGAAG | 410 | mG*mGACUGGUAmUUUmGUGUCUGAAGCUmGUUGmUmAmGmCUCCCm |
| | | CUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021253 | 211 | UGCUGAUCCCUGCCAAGCUGAC | 411 | mU*mGCUGAUCCmCUGmCCAAGCUGACUCmGUUGmUmAmGmCUCCCm |
| | | UCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021254 | 212 | CAGCAACCCCCAGAAUUGAGAG | 412 | mC*mAGCAACCCmCCAmGAAUUGAGAGACmGUUGmUmAmGmCUCCCm |
| | | ACGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021255 | 213 | GCUUCUCCUGGUGAAGGGGCUU | 413 | mG*mCUUCUCCUmGGUmGAAGGGGCUUUUmGUUGmUmAmGmCUCCCm |
| | | UUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021256 | 214 | CCAAGUGUCUUCCAGUACGAUU | 414 | mC*mCAAGUGUCmUUCmCAGUACGAUUUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021257 | 215 | CGAUGGCCAGAGUCGUUGGCUG | 415 | mC*mGAUGGCCAmGAGmUCGUUGGCUGUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021258 | 216 | GUUUAAUAAGAAUGCUUCACGG | 416 | mG*mUUUAAUAAmGAAmUGCUUCACGGCAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021259 | 217 | UUGGAGUCAGCUUGGCAGGGAU | 417 | mU*mUGGAGUCAmGCUmUGGCAGGGAUCAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021260 | 218 | GGAAGGAGGGGGUAUAAAAGCC | 418 | mG*mGAAGGAGGmGGGmUAUAAAAGCCCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021261 | 219 | ACAGGAUGGCUUCCCUUCGACU | 419 | mA*mCAGGAUGGmCUUmCCCUUCGACUCUmGUUGmUmAmGmCUCCCm |
| | | CUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021262 | 220 | CUGGACUGGUAUUUGUGUCUGA | 420 | mC*mUGGACUGGmUAUmUUGUGUCUGAAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021263 | 221 | CUCUGAUGGUCAAAGUCCUGGA | 421 | mC*mUCUGAUGGmUCAmAAGUCCUGGAUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021264 | 222 | GAAGGAGUGUACAGAGUAGAAC | 422 | mG*mAAGGAGUGmUACmAGAGUAGAACUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021265 | 223 | AGGUGGUUUUCACAGCCAACGA | 423 | mA*mGGUGGUUUmUCAmCAGCCAACGACUmGUUGmUmAmGmCUCCCm |
| | | CUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021266 | 224 | GCCAACGACUCUGGCCAUCGCC | 424 | mG*mCCAACGACmUCUmGGCCAUCGCCACmGUUGmUmAmGmCUCCCm |
| | | ACGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021267 | 225 | UACAGCACCACGGCUGUCGUCA | 425 | mU*mACAGCACCmACGmGCUGUCGUCAGCmGUUGmUmAmGmCUCCCm |
| | | GCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021268 | 226 | ACAGCACCACGGCUGUCGUCAG | 426 | mA*mCAGCACCAmCGGmCUGUCGUCAGCAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021269 | 227 | UAAACCGUGUUAGCAGCUCAGG | 427 | mU*mAAACCGUGmUUAmGCAGCUCAGGAAmGUUGmUmAmGmCUCCCm |
| | | AAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021270 | 228 | GAAGAUGCCGUGAAGCAUUCUU | 428 | mG*mAAGAUGCCmGUGmAAGCAUUCUUAUmGUUGmUmAmGmCUCCCm |
| | | AUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021271 | 229 | GAUGCCGUGAAGCAUUCUUAUU | 429 | mG*mAUGCCGUGmAAGmCAUUCUUAUUAAmGUUGmUmAmGmCUCCCm |
| | | AAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021272 | 23 | UCCUGGUGAAGGGGCUUUUAUA | 430 | mU*mCCUGGUGAmAGGmGGCUUUUAUACCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021273 | 231 | UUCUCCUGGUGAAGGGGCUUUU | 431 | mU*mUCUCCUGGmUGAmAGGGGCUUUUAUmGUUGmUmAmGmCUCCCm |
| | | AUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021274 | 232 | CUUCUCCUGGUGAAGGGGCUUU | 432 | mC*mUUCUCCUGmGUGmAAGGGGCUUUUAmGUUGmUmAmGmCUCCCm |
| | | UAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021275 | 233 | UCUACAGCAGGGCUGCCUCGGA | 433 | mU*mCUACAGCAmGGGmCUGCCUCGGACAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021276 | 234 | UUUGGUGUCCAGUUCUACUCUG | 434 | mU*mUUGGUGUCmCAGmUUCUACUCUGUAmGUUGmUmAmGmCUCCCm |
| | | UAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021277 | 235 | CAGGGCUGCGAUGGUGUAGUGG | 435 | mC*mAGGGCUGCmGAUmGGUGUAGUGGCGmGUUGmUmAmGmCUCCCm |
| | | CGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021278 | 236 | GAUGCUACUGCUUUGGCAAGAU | 436 | mG*mAUGCUACUmGCUmUUGGCAAGAUCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021279 | 237 | CUGACAGGAUGGCUUCCCUUCG | 437 | mC*mUGACAGGAmUGGmCUUCCCUUCGACmGUUGmUmAmGmCUCCCm |
| | | ACGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021280 | 238 | GCGGAGUCUGGAGAGCUGCACG | 438 | mG*mCGGAGUCUmGGAmGAGCUGCACGGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021281 | 239 | GCCACUACACCAUCGCAGCCCU | 439 | mG*mCCACUACAmCCAmUCGCAGCCCUGCmGUUGmUmAmGmCUCCCmU |
| | | GCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCmAmAmCmGCUCUmGmCCmUmUmCmUGmGC |
| | | AGAUGUGCCGCAACGCUCUGCC | | mAmUC*mG*mU*mU |
| | | UUCUGGCAUCGUU | | |
| G021282 | 240 | CCUACAGCACCACGGCUGUCGU | 440 | mC*mCUACAGCAmCCAmCGGCUGUCGUCAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021283 | 241 | CUACAGCACCACGGCUGUCGUC | 441 | mC*mUACAGCACmCACmGGCUGUCGUCAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021284 | 242 | CCCCCUCCUUCCAACCCAGGCU | 442 | mC*mCCCCUCCUmUCCmAACCCAGGCUGCmGUUGmUmAmGmCUCCCmU |
| | | GCGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCmAmAmCmGCUCUmGmCCmUmUmCmUGmGC |
| | | AGAUGUGCCGCAACGCUCUGCC | | mAmUC*mG*mU*mU |
| | | UUCUGGCAUCGUU | | |
| G021285 | 243 | AAGGGGCUUUUAUACCCCCUCC | 443 | mA*mAGGGGCUUmUUAmUACCCCCUCCUUmGUUGmUmAmGmCUCCCm |
| | | UUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021286 | 244 | GAGCUUGUGGAUCUGUGUGACG | 444 | mG*mAGCUUGUGmGAUmCUGUGUGACGGCmGUUGmUmAmGmCUCCCm |
| | | GCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021287 | 245 | ACCGCGGGGCCAGCUUCAGACA | 445 | mA*mCCGCGGGGmCCAmGCUUCAGACACAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021288 | 246 | AUCGCUCACAGGAUCACUCACC | 446 | mA*mUCGCUCACmAGGmAUCACUCACCGCmGUUGmUmAmGmCUCCCm |
| | | GCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021289 | 247 | UUUUACAGCCACGUCUACAGCA | 447 | mU*mUUUACAGCmCACmGUCUACAGCAGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021290 | 248 | UCCCAUGGUGGGCUUUCUACAA | 448 | mU*mCCCAUGGUmGGGmCUUUCUACAAGCmGUUGmUmAmGmCUCCCm |
| | | GCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021291 | 249 | GUGGUGAGCCCGUGCAGCUCUC | 449 | mG*mUGGUGAGCmCCGmUGCAGCUCUCCAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021292 | 250 | CUCAUCUGUGGUGAGCCCGUGC | 450 | mC*mUCAUCUGUmGGUmGAGCCCGUGCAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021293 | 251 | UUCUACAAACUUCUCAUCUGUG | 451 | mU*mUCUACAAAmCUUmCUCAUCUGUGGUmGUUGmUmAmGmCUCCCm |
| | | GUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021294 | 252 | CUUCUACAAACUUCUCAUCUGU | 452 | mC*mUUCUACAAmACUmUCUCAUCUGUGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021295 | 253 | AUCCGCGAAUUCAUGGAACGGG | 453 | mA*mUCCGCGAAmUUCmAUGGAACGGGGAmGUUGmUmAmGmCUCCCm |
| | | GAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021296 | 254 | CCAAACAACUUGGUGUGUCCAC | 454 | mC*mCAAACAACmUUGmGUGUGUCCACUUmGUUGmUmAmGmCUCCCm |
| | | UUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021297 | 255 | UCUCAAUUCUGGGGGUUGCUGA | 455 | mU*mCUCAAUUCmUGGmGGGUUGCUGACGmGUUGmUmAmGmCUCCCm |
| | | CGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021298 | 256 | GCUACUGCUUUGGCAAGAUCCU | 456 | mG*mCUACUGCUmUUGmGCAAGAUCCUGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021299 | 257 | UUCUUUCUUUAAAAAGUUUGAU | 457 | mU*mUCUUUCUUmUAAmAAAGUUUGAUCGmGUUGmUmAmGmCUCCCm |
| | | CGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | | | CmAmUC*mG*mU*mU |
| | | AGAUGUGCCGCAACGCUCUGCC | | |
| | | UUCUGGCAUCGUU | | |
| G021300 | 258 | CCAUGACCUCCCCAAGAUGGAA | 458 | mC*mCAUGACCUmCCCmCAAGAUGGAAAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021301 | 259 | GCAAGUUGCCAGGAGCACCCAU | 459 | mG*mCAAGUUGCmCAGmGAGCACCCAUGAmGUUGmUmAmGmCUCCCm |
| | | GAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021302 | 260 | AGCAAGUUGCCAGGAGCACCCA | 460 | mA*mGCAAGUUGmCCAmGGAGCACCCAUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021303 | 261 | UCAAGCAAGUUGCCAGGAGCAC | 461 | mU*mCAAGCAAGmUUGmCCAGGAGCACCCmGUUGmUmAmGmCUCCCm |
| | | CCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021304 | 262 | UCUUCCUCAAGCAAGUUGCCAG | 462 | mU*mCUUCCUCAmAGCmAAGUUGCCAGGAmGUUGmUmAmGmCUCCCm |
| | | GAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021305 | 263 | AUCUUCCUCAAGCAAGUUGCCA | 463 | mA*mUCUUCCUCmAAGmCAAGUUGCCAGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021306 | 264 | UGGUCCACUCUGCUUUCUGACC | 464 | mU*mGGUCCACUmCUGmCUUUCUGACCUAmGUUGmUmAmGmCUCCCm |
| | | UAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021307 | 265 | UGAACGGUUGGUCCACUCUGCU | 465 | mU*mGAACGGUUmGGUmCCACUCUGCUUUmGUUGmUmAmGmCUCCCm |
| | | UUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021308 | 266 | CAGAUUGUGUGCUUUUAAUAGC | 466 | mC*mAGAUUGUGmUGCmUUUUAAUAGCUAmGUUGmUmAmGmCUCCCm |
| | | UAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021309 | 267 | UGGGAAAACACUACUGUGCAUC | 467 | mU*mGGGAAAACmACUmACUGUGCAUCUAmGUUGmUmAmGmCUCCCm |
| | | UAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021310 | 268 | AGGAAGAUAGGUCAGAAAGCAG | 468 | mA*mGGAAGAUAmGGUmCAGAAAGCAGAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021311 | 269 | CUGGGAAAACACUACUGUGCAU | 469 | mC*mUGGGAAAAmCACmUACUGUGCAUCUmGUUGmUmAmGmCUCCCm |
| | | CUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021312 | 270 | GGCCUACUUUUCAGUAUUGUGA | 470 | mG*mGCCUACUUmUUCmAGUAUUGUGAUUmGUUGmUmAmGmCUCCCm |
| | | UUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021313 | 271 | UGGCCUACUUUUCAGUAUUGUG | 471 | mU*mGGCCUACUmUUUmCAGUAUUGUGAUmGUUGmUmAmGmCUCCCm |
| | | AUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021314 | 272 | UUAGUCACUAAAGGUUAUAAAA | 472 | mU*mUAGUCACUmAAAmGGUUAUAAAAAAmGUUGmUmAmGmCUCCCm |
| | | AAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021315 | 273 | UUUAGUCACUAAAGGUUAUAAA | 473 | mU*mUUAGUCACmUAAmAGGUUAUAAAAAmGUUGmUmAmGmCUCCCm |
| | | AAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021316 | 274 | CCUGGGUUGGAAGGAGGGGGUA | 474 | mC*mCUGGGUUGmGAAmGGAGGGGGUAUAmGUUGmUmAmGmCUCCCm |
| | | UAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021317 | 275 | CUGGGUUGGAAGGAGGGGGUA | 475 | mC*mUGGGUUGGmAAGmGAGGGGGUAUAAmGUUGmUmAmGmCUCCCm |
| | | UAAGUUGUAGCUCCCUGAAACC | | UmGmAmAmAmCmCGUUmGmCUAmCAAU*AAGmGmCCmGmUmCmGmA |
| | | GUUGCUACAAUAAGGCCGUCGA | | mAmAmGmAmUGUGCmCGCmAmAmCmGCUCUmGmCCmUmUmCmUGmG |
| | | AAGAUGUGCCGCAACGCUCUGC | | CmAmUC*mG*mU*mU |
| | | CUUCUGGCAUCGUU | | |
| G021318 | 276 | GGUAUAAAAGCCCCUUCACCAG | 476 | mG*mGUAUAAAAmGCCmCCUUCACCAGGAmGUUGmUmAmGmCUCCCm |
| | | GAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | | | CmAmUC*mG*mU*mU |
| | | AGAUGUGCCGCAACGCUCUGCC | | |
| | | UUCUGGCAUCGUU | | |
| G021319 | 277 | UUUGCUUGUAAAACAUUGAACG | 477 | mU*mUUGCUUGUmAAAmACAUUGAACGGUmGUUGmUmAmGmCUCCCm |
| | | GUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021320 | 278 | CUUCACCAGGAGAAGCCGUCAC | 478 | mC*mUUCACCAGmGAGmAAGCCGUCACACmGUUGmUmAmGmCUCCCm |
| | | ACGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021321 | 279 | GAGAAGCCGUCACACAGAUCCA | 479 | mG*mAGAAGCCGmUCAmCACAGAUCCACAmGUUGmUmAmGmCUCCCm |
| | | CAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021322 | 280 | UGCCCAGGGUGCUGGAGAAUCC | 480 | mU*mGCCCAGGGmUGCmUGGAGAAUCCAAmGUUGmUmAmGmCUCCCm |
| | | AAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021323 | 281 | GACGUGGCUGUAAAAGUGUUCA | 481 | mG*mACGUGGCUmGUAmAAAGUGUUCAAAmGUUGmUmAmGmCUCCCm |
| | | AAGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021324 | 282 | CAAAAAGACCUCUGAGGGAUCC | 482 | mC*mAAAAAGACmCUCmUGAGGGAUCCUGmGUUGmUmAmGmCUCCCm |
| | | UGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021325 | 283 | AAAAAGACCUCUGAGGGAUCCU | 483 | mA*mAAAAGACCmUCUmGAGGGAUCCUGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021326 | 284 | CUCUGGACUUGUGUGCCCUCCA | 484 | mC*mUCUGGACUmUGUmGUGCCCUCCAGGmGUUGmUmAmGmCUCCCm |
| | | GGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021327 | 285 | UCCUUCCAACCCAGGCUGCUGA | 485 | mU*mCCUUCCAAmCCCmAGGCUGCUGAUCmGUUGmUmAmGmCUCCCm |
| | | UCGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021328 | 286 | CCCCUCCUUCCAACCCAGGCUG | 486 | mC*mCCCUCCUUmCCAmACCCAGGCUGCUmGUUGmUmAmGmCUCCCmU |
| | | CUGUUGUAGCUCCCUGAAACCG | | mGmAmAmAmCmCGUUmGmCUAmCAAU*AAGmGmCCmGmUmCmGmAm |
| | | UUGCUACAAUAAGGCCGUCGAA | | AmAmGmAmUGUGCmCGCmAmAmCmGCUCUmGmCCmUmUmCmUGmGC |
| | | AGAUGUGCCGCAACGCUCUGCC | | mAmUC*mG*mU*mU |
| | | UUCUGGCAUCGUU | | |
| G021329 | 287 | AGCAACCCCCAGAAUUGAGAGA | 487 | mA*mGCAACCCCmCAGmAAUUGAGAGACUmGUUGmUmAmGmCUCCCm |
| | | CUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021330 | 288 | ACCAGGAUCUUGCCAAAGCAGU | 488 | mA*mCCAGGAUCmUUGmCCAAAGCAGUAGmGUUGmUmAmGmCUCCCm |
| | | AGGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021331 | 289 | UUGCCAAAGCAGUAGCAUCCCA | 489 | mU*mUGCCAAAGmCAGmUAGCAUCCCAUUmGUUGmUmAmGmCUCCCm |
| | | UUGUUGUAGCUCCCUGAAACCG | | UmGmAmAmAmCmCGUUmGmCUAmCAAU*AAGmGmCCmGmUmCmGmA |
| | | UUGCUACAAUAAGGCCGUCGAA | | mAmAmGmAmUGUGCmCGCmAmAmCmGCUCUmGmCCmUmUmCmUGmG |
| | | AGAUGUGCCGCAACGCUCUGCC | | CmAmUC*mG*mU*mU |
| | | UUCUGGCAUCGUU | | |
| G021536 | 290 | CCAAGUGUCUUCCAGUACGAUU | 490 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGCUCUmGmCCmUmUm |
| | | AGAUGUGCCGCAACGCUCUGCC | | CmUGGCAUCG*mU*mU |
| | | UUCUGGCAUCGUU | | |
| G021845 | 291 | CUUCACCAGGAGAAGCCGUCAC | 491 | mC*mU*mU*mCmACCmAmGGmAGmAAGCCmGUCAmCACmGUUGmUmA |
| | | ACGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGCUCUmGmCCmUmUm |
| | | AGAUGUGCCGCAACGCUCUGCC | | CmUGGCAUCG*mU*mU |
| | | UUCUGGCAUCGUU | | |
| G020927 | 292 | AUCACGAUGCCUUUAUAGGGCA | 492 | mA*mU*mC*UGCCUUUAUAGGGCACCGUUUUAGAmGmCmUmAmGmAm |
| | | CCGUUUUAGAGCUAGAAAUAGC | | AmAmUmAmGmCAAGUUAAAAUAAGGCUAGUCCGUUAUCAmAmCmUmU |
| | | AAGUUAAAAUAAGGCUAGUCCG | | mGmAmAmAmAmAmGmUmGmGmCmAmCmCmGmAmGmUmCmGmGmUm |
| | | UUAUCAACUUGAAAAAGUGGCA | | GmCmU*mU*mU*mU |
| | | CCGAGUCGGUGCUUUU | | |
| G020928 | 293 | AGUGAUAUCACGAUGCCUUUAU | 493 | mA*mG*mU*CACGAUGCCUUUAUAGGUUUUAGAmGmCmUmAmGmAmA |
| | | AGGUUUUAGAGCUAGAAAUAGC | | mAmUmAmGmCAAGUUAAAAUAAGGCUAGUCCGUUAUCAmAmCmUmU |
| | | AAGUUAAAAUAAGGCUAGUCCG | | mGmAmAmAmAmAmGmUmGmGmCmAmCmCmGmAmGmUmCmGmGmUm |
| | | UUAUCAACUUGAAAAAGUGGCA | | GmCmU*mU*mU*mU |
| | | CCGAGUCGGUGCUUUU | | |
| G020929 | 294 | GGUGCAAGGAAUGAGAACCGUU | 494 | mG*mG*mU*UGCAAGGAAUGAGAACCGUUUUAGAmGmCmUmAmGmAm |
| | | UUAGAGCUAGAAAUAGCAAGUU | | AmAmUmAmGmCAAGUUAAAAUAAGGCUAGUCCGUUAUCAmAmCmUmU |
| | | AAAAUAAGGCUAGUCCGUUAUC | | mGmAmAmAmAmAmGmUmGmGmCmAmCmCmGmAmGmUmCmGmGmUm |
| | | AACUUGAAAAAGUGGCACCGAG | | GmCmU*mU*mU*mU |
| | | UCGGUGCUUUU | | |
| G029377 | 290 | CCAAGUGUCUUCCAGUACGAUU | 931 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAUAAGmGmCCmG |
| | | UUGCUACAAUAAGGCCGUCGAA | | mUmCmGmAmAmAmGmAmUGUGCmCGmCAAmCGCUCUmGmCCmUmUm |
| | | AGAUGUGCCGCAACGCUCUGCC | | CmUGGCAUCG*mU*mU |
| | | UUCUGGCAUCGUU | | |
| G029378 | 290 | CCAAGUGUCUUCCAGUACGAUU | 932 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCmAmAUAAGmGmCC |
| | | UUGCUACAAUAAGGCCGUCGAA | | mGmUmCmGmAmAmAmGmAmUGUGCmCGmCAAmCGCUCUmGmCCmUm |
| | | AGAUGUGCCGCAACGCUCUGCC | | UmCmUGGCAUCG*mU*mU |
| | | UUCUGGCAUCGUU | | |
| G029379 | 290 | CCAAGUGUCUUCCAGUACGAUU | 933 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAmAUAAGmGmCCm |
| | | UUGCUACAAUAAGGCCGUCGAA | | GmUmCmGmAmAmAmGmAmUGUGCmCGmCAAmCGCUCUmGmCCmUmU |
| | | AGAUGUGCCGCAACGCUCUGCC | | mCmUGGCAUCG*mU*mU |
| | | UUCUGGCAUCGUU | | |
| G029380 | 290 | CCAAGUGUCUUCCAGUACGAUU | 934 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCmAmAU*AAGmGmCC |
| | | UUGCUACAAUAAGGCCGUCGAA | | mGmUmCmGmAmAmAmGmAmUGUGCmCGmCAAmCGCUCUmGmCCmUm |
| | | AGAUGUGCCGCAACGCUCUGCC | | UmCmUGGCAUCG*mU*mU |
| | | UUCUGGCAUCGUU | | |
| G029381 | 290 | CCAAGUGUCUUCCAGUACGAUU | 935 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAdTAAGmGmCCmG |
| | | UUGCUACAAUAAGGCCGUCGAA | | mUmCmGmAmAmAmGmAmUGUGCmCGmCAAmCGCUCUmGmCCmUmUm |
| | | AGAUGUGCCGCAACGCUCUGCC | | CmUGGCAUCG*mU*mU |
| | | UUCUGGCAUCGUU | | |
| G029382 | 290 | CCAAGUGUCUUCCAGUACGAUU | 936 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCmGCUCUmGmCCmUmU |
| | | AGAUGUGCCGCAACGCUCUGCC | | mCmUGGCAUCG*mU*mU |
| | | UUCUGGCAUCGUU | | |
| G029383 | 290 | CCAAGUGUCUUCCAGUACGAUU | 937 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGCUCmUmGmCCmUmU |
| | | AGAUGUGCCGCAACGCUCUGCC | | mCmUGGCAUCG*mU*mU |
| | | UUCUGGCAUCGUU | | |
| G029384 | 290 | CCAAGUGUCUUCCAGUACGAUU | 938 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGmCmUmCmUmGmCCm |
| | | AGAUGUGCCGCAACGCUCUGCC | | UmUmCmUGGCAUCG*mU*mU |
| | | UUCUGGCAUCGUU | | |
| G029385 | 290 | CCAAGUGUCUUCCAGUACGAUU | 939 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGmCmUmCUmGmCCmU |
| | | AGAUGUGCCGCAACGCUCUGCC | | mUmCmUGGCAUCG*mU*mU |
| | | UUCUGGCAUCGUU | | |
| G029386 | 290 | CCAAGUGUCUUCCAGUACGAUU | 940 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCmGmCmUmCmUmGmCC |
| | | AGAUGUGCCGCAACGCUCUGCC | | mUmUmCmUGGCAUCG*mU*mU |
| | | UUCUGGCAUCGUU | | |
| G029387 | 290 | CCAAGUGUCUUCCAGUACGAUU | 941 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCmAmAmCmGmCmUmCmUmGm |
| | | AGAUGUGCCGCAACGCUCUGCC | | CmCmUmUmCmUGGCAUCG*mU*mU |
| | | UUCUGGCAUCGUU | | |
| G029388 | 290 | CCAAGUGUCUUCCAGUACGAUU | 942 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGCUCUmGmCCmUmUm |
| | | AGAUGUGCCGCAACGCUCUGCC | | CmUGGCAUmCmG*mU*mU |
| | | UUCUGGCAUCGUU | | |
| G029389 | 290 | CCAAGUGUCUUCCAGUACGAUU | 943 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCGCUCUmGmCCmUmUm |
| | | AGAUGUGCCGCAACGCUCUGCC | | CmUGGCmAmUmCmG*mU*mU |
| | | UUCUGGCAUCGUU | | |
| G029390 | 290 | CCAAGUGUCUUCCAGUACGAUU | 944 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCmAmAmCmGmCmUmCmUmGm |
| | | AGAUGUGCCGCAACGCUCUGCC | | CmCmUmUmCmUGGCmAmUmCmG*mU*mU |
| | | UUCUGGCAUCGUU | | |
| G029391 | 290 | CCAAGUGUCUUCCAGUACGAUU | 945 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCmAmAmCmGmCmUmCmUmGm |
| | | AGAUGUGCCGCAACGCUCUGCC | | CmCmUmUmCmUGGCAUmCmG*mU*mU |
| | | UUCUGGCAUCGUU | | |
| G029392 | 290 | CCAAGUGUCUUCCAGUACGAUU | 946 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAU*AAGmGmCCmG |
| | | UUGCUACAAUAAGGCCGUCGAA | | mUmCmGmAmAmAmGmAmUGUGCmCGmCAAmCmGmCmUmCmUmGmCC |
| | | AGAUGUGCCGCAACGCUCUGCC | | mUmUmCmUGGCAUmCmG*mU*mU |
| | | UUCUGGCAUCGUU | | |
| G032572 | 290 | CCAAGUGUCUUCCAGUACGAUU | 951 | mC*mC*mA*mAmGUGmUmCUmUCmCAGUAmCGAUmUUGmGUUGmUmA |
| | | UGGUUGUAGCUCCCUGAAACCG | | mGmCUCCCmUmGmAmAmAmCmCGUUmGmCUAmCAAUAAGmGmCCmG |
| | | UUGCUACAAUAAGGCCGUCGAA | | mUmCmGmAmAmAmGmAmUGUGCmCGmCAAmCGmCmUmCmUmGmCCm |
| | | AGAUGUGCCGCAACGCUCUGCC | | UmUmCmUGGCAUCG*mU*mU |
| | | UUCUGGCAUCGUU | | |
| G031771 | 295 | CCAAGUGUCUUCCAGUACGAUU | 952 | mC*mC*mA*mAmGUGmUmCUmUCmCAGUAmCGAUmUUGmGUUGmUmA |
| | | UGGUUGUAGCUCCCUUCGAAAG | | mGmCUCCCmUmUmCmGmAmAmAmGmAmCmCGUUmGmCUAmCAAUAA |
| | | ACCGUUGCUACAAUAAGGCCGU | | GmGmCCmGmUmCmGmAmAmAmGmAmUGUGCmCGmCAAmCGmCmUmC |
| | | CGAAAGAUGUGCCGCAACGCUC | | mUmGmCCmUmUmCmUGGCAUCG*mU*mU |
| | | UGCCUUCUGGCAUCGUU | | |

N represents a nucleotide having any base, e.g., A, C, G, or U. (mN*)₃ represents three consecutive nucleotides each having any base, a 2′-OMe, and a 3′ PS linkage to the next nucleotide, respectively. (N)₂₀₋₂₅ represent 20-25, i.e., 20, 21, 22, 23, 24, or 25 consecutive N. A, C, G, and U represent nucleotides having adenine, cytosine, guanine, and uracil bases, respectively.

Nucleotide modifications are indicated in Tables 1-2 as follows: m: 2′-OMe; *: PS linkage; f: 2′-fluoro; (invd): inverted abasic; moe: 2′-moe; e: ENA; d: deoxyribonucleotide (also note that T is always a deoxyribonucleotide); x: UNA. In the sgRNA modified sequences, in certain embodiments, each A, C, G, U, and N is independently a ribose sugar (2′-OH). In certain embodiments, each A, C, G, U, and N is a ribose sugar (2′-OH). Thus, for example, mA represents 2′-O-methyl adenosine; xA represents a UNA nucleotide with an adenine nucleobase; eA represents an ENA nucleotide with an adenine nucleobase; and dA represents an adenosine deoxyribonucleotide.

sgRNA designations are sometimes provided with one or more leading zeroes immediately following the G. This does not affect the meaning of the designation. Thus, for example, G000282, G0282, G00282, and G282 refer to the same sgRNA.

TABLE 3
exemplary NmeCas9 sgRNA (SEQ ID NO: 500)

| 1-24 | 25 | 26 | 27 | 28 | 29 | 30 | 31 |
|---|---|---|---|---|---|---|---|
| NNNNNNNNNNNNNNNNNNNNNNNN | G | U | U | G | U | A | G |

| | | | | | | Lower stem | | | | | | | | | | | |
|---|---|---|---|---|---|---|---|---|---|---|---|---|---|---|---|---|---|
| Guide region | | | | | | Repeat/Anti-Repeat region | | | | | | | | | | | |
| | 32 | 33 | 34 | 35 | 36 | 37 | 38 | 39 | 40 | 41 | 42 | 43 | 44 | 45 | 46 | 47 | 48 |
| | C | U | C | C | C | U | U | U | C | U | C | A | U | U | U | C | G |

| | |
|---|---|
| Lower stem | Upper stem |

Repeat/Anti-Repeat region

| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 | 77 |
|---|---|---|---|---|---|---|---|---|---|---|---|---|---|---|---|---|---|---|---|---|---|---|---|---|---|---|---|---|
| G | A | A | A | C | G | A | A | A | U | G | A | G | A | A | C | C | G | U | U | G | C | U | A | C | A | A | U | A |

| | | |
|---|---|---|
| Loop | Upper stem | Lower Stem |

Repeat/Anti-Repeat region

| 78 | 79 | 80 | 81 | 82 | 83 | 84 | 85 | 86 | 87 | 88 | 89 | 90 | 91 | 92 | 93 | 94 |
|---|---|---|---|---|---|---|---|---|---|---|---|---|---|---|---|---|
| A | G | G | C | C | G | U | C | U | G | A | A | A | A | G | A | U |

| | | |
|---|---|---|
| Stem | Loop | Stem (96: unpaired) |

Hairpin 1

| 95 | 96 | 97 | 98 | 99 | 100 | 101 | 102 | 103 | 104 | 105 | 106 | 107 | 108 |
|---|---|---|---|---|---|---|---|---|---|---|---|---|---|
| G | U | G | C | C | G | C | A | A | C | G | C | U | C |

| | | |
|---|---|---|
| Stem (96: unpaired) | Lower stem | Bulge |

| | |
|---|---|
| Hairpin 1 | Hairpin 2 |

| 109 | 110 | 111 | 112 | 113 | 114 | 115 | 116 | 117 | 118 | 119 | 120 | 121 |
|---|---|---|---|---|---|---|---|---|---|---|---|---|
| U | G | C | C | C | C | U | U | A | A | A | G | C |

Upper Stem
Hairpin 2

| 122 | 123 | 124 | 125 | 126 | 127 | 128 | 129 | 130 | 131 | 132 | 133 | 134 |
|---|---|---|---|---|---|---|---|---|---|---|---|---|
| U | U | C | U | G | C | U | U | U | A | A | G | G |

| | |
|---|---|
| Loop | Upper Stem |

| | | | Hairpin 2 | | | | | | | |
|---|---|---|---|---|---|---|---|---|---|---|
| 135 | 136 | 137 | 138 | 139 | 140 | 141 | 142 | 143 | 144 | 145 |
| G | G | C | A | U | C | G | U | U | U | A |

| | | |
|---|---|---|
| Upper Stem | Bulge | Lower Stem |

| | |
|---|---|
| Hairpin 2 | Tail |

### Definitions

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to,” such that recitation of items in a list is not to the exclusion of other like items that can be substituted or added to the listed items.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means +10%. In certain embodiments, about means +5%, +2%, or +1%. When about is present before a series of numbers or a range, it is understood that “about” can modify each of the numbers in the series or range. At the very least, and not as an attempt to limit the application of the doctrine of equivalents to the scope of the claims, each numerical parameter should at least be construed in light of the number of reported significant digits and by applying ordinary rounding techniques.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20 nucleotide nucleic acid molecule” means that 17, 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has 2, 1, or 0 nucleotide base pairs. When “no more than” or “less than” is present before a series of numbers or a range, it is understood that each of the numbers in the series or range is modified.

As used herein, ranges include both the upper and lower limits.

As used herein, it is understood that when the maximum amount of a value is represented by 100% (e.g., 100% inhibition) that the value is interpreted in light of the method of detection. For example, 100% inhibition is understood as inhibition to a level below the level of detection of the assay.

“Editing efficiency” or “editing percentage” or “percent editing” as used herein is the total number of sequence reads with insertions, deletions, or base changes of nucleotides into the target region of interest over the total number of sequence reads following cleavage or nicking by a Cas RNP.

“Regions” as used herein describes portions of nucleic acids. Regions may also be referred to as “modules” or “domains.” Regions of an sgRNA may perform particular functions, e.g., in directing endonuclease activity of the RNP, for example as described in Briner A E et al., Molecular Cell 56:333-339 (2014), or have predicted structures. Exemplary regions of an sgRNA are described in Table 3.

“Hairpin” or “hairpin structure” as used herein describes a duplex of nucleic acids that is created when a nucleic acid strand folds and forms base pairs with another section of the same strand. A hairpin may form a structure that comprises a loop or a U-shape. In some embodiments, a hairpin may be comprised of an RNA loop. Hairpins can be formed with two complementary sequences in a single nucleic acid molecule bind together, with a folding or wrinkling of the molecule. In some embodiments, hairpins comprise stem or stem loop structures. In some embodiments, a hairpin comprises a loop and a stem. As used herein, when two hairpins are present in a gRNA, a “hairpin region” can refer to hairpin 1 and hairpin 2 and the intervening sequence (e.g., “n”) between hairpin 1 and hairpin 2 of a conserved region of an sgRNA.

As used herein, “form a duplex portion” is understood as being capable of forming an uninterrupted duplex portion or predicted to form an uninterrupted duplex portion, e.g., by base pairing. A duplex portion may comprise two complementary sequences, e.g., a first hairpin stem region and a second hairpin stem region complementary to the first. As used herein, a duplex portion has a length of at least 2 base pairs. A duplex portion optionally comprises 2-10 base pairs, and the two strands that form the duplex portion may be joined, for example, by a nucleotide loop. Base pairing in a duplex can include Watson-Crick base pairing, optionally in combination with base stacking. As used herein, a duplex portion can include a single nucleotide discontinuity on one strand wherein each contiguous nucleotide on one strand is based paired with a nucleotide on the complementary strand which may have a discontinuity of one non-base paired nucleotide, e.g., as in nucleotide 96 of SEQ ID NO: 500 in hairpin 1, wherein the discontinuity is flanked immediately 5′ and 3′ with Watson-Crick base pairs. This is distinct from non-paired nucleotides 36 and 65 in the repeat-anti-repeat region, and non-paired nucleotides 106-108 and 139 in hairpin 2, which constitute a discontinuity resulting in two duplex portions, as defined herein. RNA structures are well known in the art and tools are available for structural prediction of RNAs (see, e.g., Sato et al., Nature Comm. 12:941 (2021); RNAstructure at ma.urmc.rochester.edu/RNAstructureWeb/Servers/Predict1/Predicti.html and RNAfold WebServer at ma.tbi.univie.ac.at/cgi-bin/RNAWebSuite/RNAfold.cgi). Bridging lengths and structural flexibility required to permit a fold and form a loop to allow nucleobases to come into sufficiently close proximity to base pair are well known in the art.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases (which have double strand cleaving activity), Cas nickases (which have single strand cleaving activity), and inactivated forms thereof (“dCas DNA binding agents”). “Cas nuclease”, as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g., via fusion with a FokI domain. In some embodiments, the RNA-guided DNA binding agent has nuclease activity, e.g., cleavase or nickase activity.

Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, amino acid sequences, or nucleic acid sequences encoding the amino acid sequences provided herein are also contemplated. Exemplary open reading frames for Cas9 are provided in Table 4A.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

“Stem loop” as used herein describes a secondary structure of nucleotides that form a base-paired “stem” that ends in a loop of unpaired nucleic acids. A stem may be formed when two regions of the same nucleic acid strand are at least partially complementary in sequence when read in opposite directions. “Loop” as used herein describes a region of nucleotides that do not base pair (i.e., are not complementary) that may cap a stem. A “tetraloop” describes a loop of 4 nucleotides. As used herein, the upper stem of an sgRNA may comprise a tetraloop.

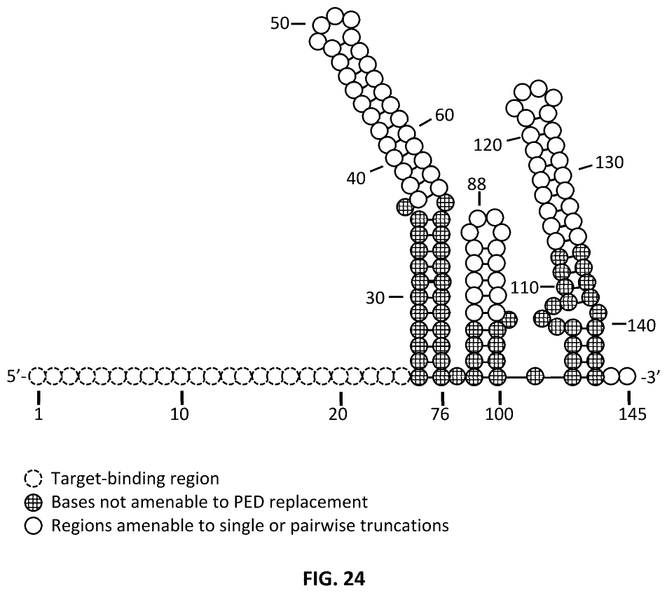

“Guide RNA”, “gRNA”, and “guide” are used herein interchangeably to refer to, the combination of a crRNA (also known as CRISPR RNA) and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally occurring sequence, or a trRNA sequence with modifications or variations compared to naturally-occurring sequences. Guide RNAs can include modified RNAs as described herein. In some embodiments, a guide RNA as used herein does not include a non-nucleotide linker to join two nucleotides within the guide RNA. Unless otherwise clear from the context, guide RNAs described herein are suitable for use with an Nine Cas9, e.g., an Nme1, Nme2, or Nme3 Cas9. For example, FIG. 24 shows an exemplary schematic of Nme2 sgRNA in a possible secondary structure.

As used herein, a nucleotide that is, for example, 6 nucleotides from the 5′ end of a particular sgRNA segment is the sixth nucleotide of that segment, or “nucleotide 6” from the 5′ end, e.g., XXXXXN, where N is the 6^{th} nucleotide from the 5′ end. A range of nucleotides that is located “at or after” 6 nucleotides from the 5′ end begins with the 6^{th} nucleotide and continues down the chain toward the 3′ end. Similarly, a nucleotide that is, for example, 5 nucleotides from the 3′ end of the chain is the 5^{th} nucleotide when counting from the 3′ end of the chain, e.g., NXXXX. A numeric position or range in the guide region refers to the position as determined from the 5′ end unless another point of reference is specified; for example, “nucleotide 5” in a guide region is the 5′ nucleotide from the 5′ end.

The term a “conserved region” refers to a conserved region of an N. meningitidis Cas9 (“NmeCas9”) gRNA as shown in Table 3. The first row shows the numbering of the nucleotides; the second row shows an exemplary sequence (e.g., SEQ ID NO: 500); and the third and fourth rows show the regions. Shortened conserved regions lack at least one nucleotide shown in Table 3, as discussed in detail below.

As used herein, a “shortened” region in a gRNA is a conserved region of a gRNA that lacks at least 1 nucleotide compared to the corresponding conserved region shown in Table 3. Similarly, “shortened” with respect to an sgRNA means that its conserved region comprises fewer nucleotides than the sgRNA conserved region shown in Table 3. Under no circumstances does “shortened” imply any particular limitation on a process or manner of production of the gRNA.

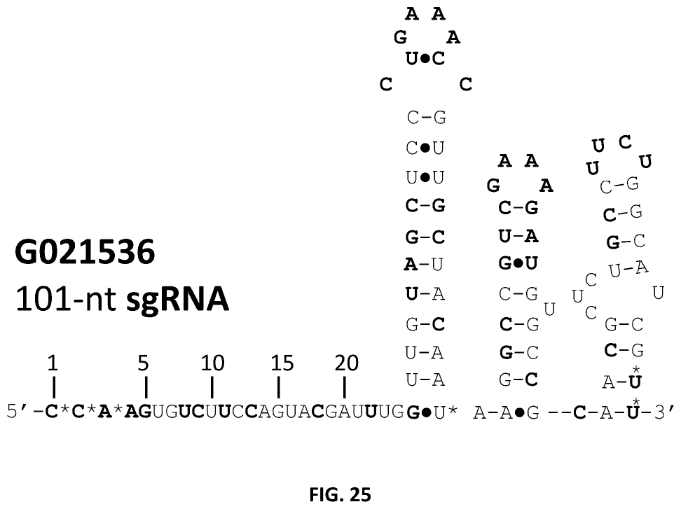

“Substituted” or “substitution” as used herein with respect to a polynucleotide refers to an alteration of a nucleobase that changes its preferred base for Watson-Crick pairing or disrupts a base stacking interaction. When a certain region of a guide RNA is “unsubstituted” as used herein, the sequence of the region can be aligned to that of the corresponding conserved region of a NmeCas9 sgRNA (e.g., SEQ ID NO: 500) or any other gRNAs (e.g., part of SEQ ID NO: 1-19, 21-42, 301-494, and 931-946) with gaps and matches only (i.e., no mismatches), where bases are considered to match if they have the same preferred standard partner base (A, C, G, or T/U) for Watson-Crick pairing or have the paired base stacking interactions as shown in FIG. 25.

As used herein, a “conservative substitution” with respect to a polynucleotide refers to an alteration of a nucleobase means exchanging positions of base paired nucleotides such that base pairings may be maintained. For example, a G-C pair becomes a C-G pair, an A-U pair for a U-A pair, or other natural or modified base pairing.

As used herein, “substituted” and the like, in regard to unpaired nucleotides (e.g., loops of the repeat/anti-repeat, hairpin 1, or hairpin 2 regions, i.e., nucleotides 49-52, 87-90, and 122-125 in SEQ ID NO: 500, respectively, or other unpaired nucleotides) refers to the replacement of one or more nucleotides, e.g., 1, 2, 3, or 4 nucleotides, of the nucleotide sequence with a different nucleotide that does not interfere with the formation of a structure by the unpaired nucleotides (e.g., a bulge or a loop) which may thus permit formation of one or more duplex portions, e.g., in the repeat/anti-repeat, hairpin 1, or hairpin 2 regions.

In some embodiments, a gRNA comprises nucleotides that “match the modification pattern” at corresponding or specified nucleotides of a gRNA described herein. This means that the nucleotides matching the modification pattern have the same modifications (e.g., phosphorothioate, 2′-fluoro, 2′-OMe, etc.) as the nucleotides at the corresponding positions of the gRNA described herein, regardless of whether the nucleobases at those positions match. For example, if in a first gRNA, nucleotides 5 and 6, respectively, have 2′-OMe and phosphorothioate modifications, then this gRNA has the same modification pattern at nucleotides 5 and 6 as a second gRNA that also has 2′-OMe and phosphorothioate modifications at nucleotides 5 and 6, respectively, regardless of whether the nucleobases at positions 5 and 6 are the same or different in the first and second gRNAs. However, a 2′-OMe modification at nucleotide 6 but not nucleotide 7 is not the same modification pattern at nucleotides 6 and 7 as a 2′-OMe modification at nucleotide 7 but not nucleotide 6. Similarly, a modification pattern that matches at least 75% of the modification pattern of a gRNA described herein means that at least 75% of the nucleotides have the same modifications as the corresponding positions of the gRNA described herein. Corresponding positions may be determined by pairwise or structural alignment.

As used herein, a “guide sequence” or “guide region” and the like refer to a sequence within a guide RNA that is complementary to a target sequence and functions to direct a guide RNA to a target sequence for binding or modification (e.g., cleavage) by NmeCas9A “guide sequence” may also be referred to as a “targeting sequence,” or a “spacer sequence.” A guide sequence can be 20-25 nucleotides in length, e.g., in the case of Nine Cas9, e.g., 20-, 21-, 22-, 23-, 24- or 25-nucleotides in length.

Target sequences for RNA-guided DNA binding agents include both the positive and negative strands of genomic DNA (i.e., the sequence given and the reverse complement of the sequence), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, the “5′ end” refers to the first nucleotide of the gRNA, including a dgRNA (typically the 5′ end of the crRNA of the dgRNA) and sgRNA, i.e., the 5′ end of the guide sequence, in which the 5′ position is not linked to another nucleotide.

As used herein, a “5′ end modification” refers to a gRNA comprising a guide region having modifications in one or more of the one (1) to about seven (7) nucleotides, optionally to about four (4) nucleotides at its 5′ end, optionally wherein the first nucleotide (from the 5′ end) of the gRNA is modified.

As used herein, the “3′ end” refers to the end or terminal nucleotide of a gRNA, in which the 3′ position is not linked to another nucleotide. In some embodiments, the 3′ end is in the 3′ tail. In some embodiments, the 3′ end is in the conserved region of a gRNA.

As used herein, a “3′ end modification” refers to a gRNA having modifications in one or more of the one (1) to about seven (7) nucleotides, optionally about four (4) nucleotides, at its 3′ end, optionally wherein the last nucleotide (i.e., the 3′ most nucleotide) of the gRNA is modified. If a 3′ tail is present, the 1 to about 7 nucleotides, optionally about four (4) nucleotides, may be within the 3′ tail. If a 3′ tail is not present, the 1 to about 7 nucleotides, optionally about four (4) nucleotides, may be within the conserved region of a sgRNA.

The “last,” “second to last,” “third to last,” etc., nucleotide refers to the 3′ most, second 3′ most, third 3′ most, etc., nucleotide, respectively in a given sequence. For example, in the sequence 5′-AAACTG-3′, the last, second to last, and third to last nucleotides are G, T, and C, respectively. The phrase “last 3 nucleotides” refers to the last, second to last, and third to last nucleotides; more generally, “last N nucleotides” refers to the last to the Nth to last nucleotides, inclusive. “Third nucleotide from the 3′ end of the 3′ terminus” is equivalent to “third to last nucleotide.” Similarly, “third nucleotide from the 5′ end of the 5′ terminus” is equivalent to “third nucleotide at the 5′ terminus.”

As used herein, a “protective end modification” (such as a protective 5′ end modification or protective 3′ end modification) refers to a modification of one or more nucleotides within seven nucleotides, optionally four nucleotides, of the end of an sgRNA that reduces degradation of the sgRNA, such as exonucleolytic degradation. In some embodiments, a protective end modification comprises modifications of at least two or at least three nucleotides within seven nucleotides, optionally four nucleotides, of the end of the sgRNA. In some embodiments, the modifications comprise phosphorothioate linkages, 2′ modifications such as 2′-OMe or 2′-fluoro, 2′-H (DNA), ENA, UNA, or a combination thereof. In some embodiments, the modifications comprise phosphorothioate linkages and 2′-OMe modifications. In some embodiments, at least three terminal nucleotides are modified, e.g., with phosphorothioate linkages or with a combination of phosphorothioate linkages and 2′-OMe modifications. In some embodiments, at least two terminal nucleotides are modified, e.g., with phosphorothioate linkages or with a combination of phosphorothioate linkages and 2′-OMe modifications. Modifications known to those of skill in the art to reduce exonucleolytic degradation are encompassed.

In some embodiments, a “3′ tail” comprising about 1-10 nucleotides, optionally about 1-4 nucleotides, following the conserved region of a sgRNA at its 3′ end.

Several Cas9 orthologs have been obtained from N. meningitidis (Esvelt et al., NAT. METHODS, vol. 10, 2013, 1116-1121; Hou et al., PNAS, vol. 110, 2013, pages 15644-15649) (Nme1Cas9, Nme2Cas9, and Nme3Cas9). The Nme2Cas9 ortholog functions efficiently in mammalian cells, recognizes an N4CC PAM, and can be used for in vivo editing (Ran et al., NATURE, vol. 520, 2015, pages 186-191; Kim et al., NAT. COMMUN., vol. 8, 2017, pages 14500). Nme2Cas9 has been shown to be naturally resistant to off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Further, NmeCas9 variants are known in the art, see, e.g., Huang et al., Nature Biotech. 2022, doi.org/10.1038/s41587-022-01410-2, which describes Cas9 variants targeting single-nucleotide-pyrimidine PAMs.

As used herein, “NmeCas9” (sometimes referred to as “Cas9”) encompasses NmeCas9, e.g., Nme1Cas9, Nme2Cas9, and Nme3Cas9; the variants of NmeCas9 listed herein, and equivalents thereof. See, e.g., Edraki et al., Mol. Cell 73:714-726, 2019. “Cas nuclease”, also called “Cas protein”, as used herein, encompasses Cas cleavases, Cas nickases which further have RNA-guided DNA cleavases or nickase activity, and dCas DNA binding agents, in which cleavase/nickase activity is inactivated. In some embodiments, NmeCas9 has double strand cleavage activity. In some embodiments, NmeCas9 has nickase activity. In some embodiments, NmeCas9 comprises a dCas DNA binding domain.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions. A modified mRNA comprises at least one nucleotide in which one or more of the phosphate, sugar, or nucleobase differ from that of a standard adenosine, cytidine, guanidine, or uridine nucleotide.

As used herein, a “subject” refers to any member of the animal kingdom. In some embodiments, “subject” refers to humans. In some embodiments, “subject” refers to non-human animals. In some embodiments, “subject” refers to primates. In some embodiment, “subject” refers to non-human primates. In some embodiments, subjects include, but are not limited to, mammals, birds, reptiles, amphibians, fish, insects, or worms. In certain embodiments, the non-human subject is a mammal (e.g., a rodent, a mouse, a rat, a rabbit, a monkey, a dog, a cat, a sheep, cattle, a primate, or a pig). In some embodiments, a subject may be a transgenic animal, genetically-engineered animal, or a clone. In certain embodiments of the present invention the subject is an adult, an adolescent or an infant. In some embodiments, terms “individual” or “patient” are used and are intended to be interchangeable with “subject” wherein the subject is a human subject.

As used herein, “treatment” refers to any administration or application of a therapeutic for disease or disorder in a subject, and includes slowing or arresting disease development or progression, relieving one or more signs or symptoms of the disease, curing the disease, or preventing reoccurrence of one or more symptoms of the disease.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

I. Guide RNAs with One or More Shortened Conserved Regions

Provided herein are guide RNAs (gRNAs) comprising one or more shortened conserved regions.

In some embodiments, a gRNA provided herein comprises a guide region and a conserved region comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the gRNA is from N. meningitidis Cas9 (NmeCas9).

In some embodiments, the conserved region comprises one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500; and
wherein at least 10 nucleotides are modified nucleotides.

In some embodiments, the conserved region comprises:
a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 500; and
(ii) nucleotide 36 is linked to nucleotide 65 by at least 2 nucleotides;
wherein one or both nucleotides 144-145 are optionally deleted relative to SEQ ID NO: 500;
wherein at least 10 nucleotides in the conserved region are modified nucleotides.

In some embodiments, the conserved region comprises:
a shortened hairpin 1 region, wherein the shortened hairpin 1 lacks 2-10, optionally 2-8 nucleotides, wherein
(i) one or more of nucleotides 82-86 and 91-95 is deleted and optionally one or more of positions 82-96 is substituted relative to SEQ ID NO: 500; and
(ii) nucleotide 81 is linked to nucleotide 96 by at least 4 nucleotides;
wherein one or both nucleotides 144-145 are optionally deleted relative to SEQ ID NO: 500;
wherein at least 10 nucleotides in the conserved region are modified nucleotides.

In some embodiments, the conserved region comprises:
a shortened hairpin 2 region, wherein the shortened hairpin 2 lacks 2-18, optionally 2-16 nucleotides, wherein
(i) one or more of nucleotides 113-121 and 126-134 is deleted and optionally one or more of nucleotides 113-134 is substituted relative to SEQ ID NO: 500; and
(ii) nucleotide 112 is linked to nucleotide 135 by at least 4 nucleotides;
wherein one or both nucleotides 144-145 are optionally deleted relative to SEQ ID NO: 500;
wherein at least 10 nucleotides in the conserved region are modified nucleotides.

In some embodiments, the conserved region comprises:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; and
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
wherein one or both nucleotides 144-145 are optionally deleted relative to SEQ ID NO: 500;
wherein at least 10 nucleotides in the conserved region are modified nucleotides.

In some embodiments, the conserved region comprises:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; and
(b) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides in the conserved region are modified nucleotides.

In some embodiments, the conserved region comprises:
(a) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; and
(b) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides in the conserved region are modified nucleotides.

In some embodiments, the conserved region comprises:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides;
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; and
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides of the conserved region are modified nucleotides.

In some embodiment, the gRNA disclosed herein is a sgRNA.

In some embodiments, one or both nucleotides 144-145 are deleted relative to SEQ ID NO: 500.

In some embodiments, at least 10 nucleotides of the conserved region are modified nucleotides.

In some embodiments, a repeat/anti-repeat region of a gRNA is a shortened repeat/anti-repeat region lacking 2-24 nucleotides, e.g., any of the repeat/anti-repeat regions indicated in the numbered embodiments above or Tables 1-2 or described elsewhere herein, which may be combined with any of the shortened hairpin 1 region or hairpin 2 region described herein, including but not limited to combinations indicated in the numbered embodiments above and represented in the sequences of Tables 1-2 or described elsewhere herein. In some embodiments, one or more of positions 49-52, 87-90, or 122-125 is substituted relative to SEQ ID NO: 500. In some embodiments, all of positions 49-52, 87-90, or 122-125 are substituted relative to SEQ ID NO: 500. In some embodiments, the 3′ tail provided in Tables 1-2 or described herein is deleted.

In some embodiments, the shortened repeat/anti-repeat region of the gRNA lacks 18 nucleotides. In some embodiments, the shortened repeat/anti-repeat region of the gRNA lacks 22 nucleotides.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotide 36 is linked to nucleotide 65 by 7 nucleotides. In some embodiments, in the shortened repeat/anti-repeat region of the gRNA, nucleotide 36 is linked to nucleotide 65 by 8 nucleotides. In some embodiments, in the shortened repeat/anti-repeat region of the gRNA, nucleotide 36 is linked to nucleotide 65 by 9 nucleotides. In some embodiments, in the shortened repeat/anti-repeat region of the gRNA, nucleotide 36 is linked to nucleotide 65 by 10 nucleotides.

In some embodiments, in the shortened repeat/anti-repeat region of the gRNA, nucleotides 38-48 and 53-63 are deleted relative to SEQ ID NO: 500. In some embodiments, in the shortened repeat/anti-repeat region of the gRNA, nucleotides 38, 41-48, 53-60, and 63 are deleted relative to SEQ ID NO: 500.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotides 38-48 and 53-63 are deleted relative to SEQ ID NO: 500, and nucleotide 36 is linked to nucleotide 65 by nucleotides 37, 49-52, and 64.

In some embodiments, in the shortened repeat/anti-repeat region of the gRNA, nucleotide 36 is linked to nucleotide 65 by 10 nucleotides. In some embodiments, in the shortened repeat/anti-repeat region of the gRNA, nucleotides 38, 41-48, 53-60, and 63 are deleted relative to SEQ ID NO: 500, and nucleotide 36 is linked to nucleotide 65 by nucleotides 37, 39, 40, 49-52, 61, 62, and 64.

In some embodiments, all of nucleotides 38-48 and nucleotides 53-63 of the upper stem of the shortened repeat/anti-repeat region are deleted relative to SEQ ID NO: 500.

In some embodiments, all of nucleotides 39-48 and nucleotides 53-62 of the upper stem of the shortened repeat/anti-repeat region are deleted relative to SEQ ID NO: 500, and nucleotides 38 and 63 is substituted.

In some embodiments, the shortened repeat/anti-repeat region has 14 modified nucleotides. In some embodiments, the shortened repeat/anti-repeat region has 15 modified nucleotides. In some embodiments, the shortened repeat/anti-repeat region has 16 modified nucleotides. In some embodiments, the shortened repeat/anti-repeat region has 17 modified nucleotides. In some embodiments, the shortened repeat/anti-repeat region has 18 modified nucleotides. In some embodiments, the shortened repeat/anti-repeat region has 19 modified nucleotides. In some embodiments, the shortened repeat/anti-repeat region has 20 modified nucleotides.

In some embodiments, the shortened hairpin 1 region lacks 2 nucleotides. In some embodiments, the shortened hairpin 1 region lacks 21 nucleotides. In some embodiments, the shortened hairpin 1 region lacks 2 nucleotides, and nucleotides 86 and 91 are deleted relative to SEQ ID NO: 500. In some embodiments, the shortened hairpin 1 region lacks 2 nucleotides, and nucleotides 85 and 92 are deleted relative to SEQ ID NO: 500. In some embodiments, in the shortened hairpin 1 region, nucleotide 81 is linked to nucleotide 96 by 12 nucleotides. In some embodiments, in the shortened hairpin 1 region, nucleotide 81 is linked to nucleotide 96 by 12 nucleotides. In some embodiments, in the shortened hairpin 1 region, nucleotides 86 and 91 are deleted relative to SEQ ID NO: 500, and nucleotide 81 is linked to nucleotide 96 by nucleotides 82-85, 87-90, and 92-95. In some embodiments, in the shortened hairpin 1 region, nucleotides 85 and 92 are deleted relative to SEQ ID NO: 500, and nucleotide 81 is linked to nucleotide 96 by nucleotides 82-84, 86-91, and 93-95.

In some embodiments, the shortened hairpin 1 region has a duplex portion of 7 base paired nucleotides in length. In some embodiments, the shortened hairpin 1 region has a duplex portion of 8 base paired nucleotides in length.

In the stem of the shortened hairpin 1 region is seven base paired nucleotides in length. In some embodiments, nucleotides 85-86 and nucleotides 91-92 of the shortened hairpin 1 region are deleted.

In some embodiments, the shortened hairpin 1 region has 13 modified nucleotides.

In some embodiments, the shortened hairpin 2 lacks 18 nucleotides. In some embodiments, the shortened hairpin 2 has 24 nucleotides. In some embodiments, in the shortened hairpin 2 nucleotides 113-121 and 126-134 are deleted relative to SEQ ID NO: 500. In some embodiments, the shortened hairpin 2 lacks 18 nucleotides, and nucleotides 113-121 and 126-134 are deleted relative to SEQ ID NO: 500. In some embodiments, in the shortened hairpin 2 region, nucleotide 112 is linked to nucleotide 135 by 4 nucleotides. In some embodiments, in the shortened hairpin 2 region, nucleotides 113-121 and 126-134 are deleted relative to SEQ ID NO: 500 and nucleotide 112 is linked to nucleotide 135 by nucleotides 122-125.

In some embodiments, the shortened repeat/anti-repeat region has a length of 28 nucleotides. In some embodiments, the shortened repeat/anti-repeat region has a length of 32 nucleotides.

In some embodiments, the upper stem of the shortened repeat/anti-repeat region comprises no more than one base pair. In some embodiments, the upper stem of the shortened repeat/anti-repeat region comprises no more than three base pairs.

In some embodiments, the shortened hairpin 2 region has 8 modified nucleotides.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 500, wherein
   (i) nucleotides 38-48 and 53-63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500; and
(c) a shortened hairpin 2 region, wherein the shortened hairpin 2 lacks 18 nucleotides, wherein nucleotides 113-121 and 126-134 are deleted relative to SEQ ID NO: 500; and wherein nucleotides 144-145 are deleted relative to SEQ ID NO: 500; wherein at least 10 nucleotides are modified nucleotides.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 500, wherein
   (i) nucleotides 38, 41-48, 53-60, and 63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500;
(c) a shortened hairpin 2 region, wherein the shortened hairpin 2 lacks 18 nucleotides, wherein nucleotides 113-121 and 126-134 are deleted relative to SEQ ID NO: 500; and
wherein nucleotides 144-145 are deleted relative to SEQ ID NO: 500;
wherein at least 10 nucleotides are modified nucleotides.

In some embodiments, a guide RNA (gRNA) is provided, the gRNA comprising a guide region and a conserved region, the conserved region comprising one or more of:
(a) a shortened repeat/anti-repeat region, wherein the shortened repeat/anti-repeat region lacks 18-22 nucleotides relative to SEQ ID NO: 500, wherein
   (i) nucleotides 37-48 and 53-64 are deleted; and
   (ii) nucleotide 36 is linked to nucleotide 65 by 6-10 nucleotides; or
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500; or
(c) a shortened hairpin 2 region, wherein the shortened hairpin 2 lacks 18 nucleotides, wherein nucleotides 113-121 and 126-134 are deleted relative to SEQ ID NO: 500; and
wherein nucleotides 144-145 are deleted relative to SEQ ID NO: 500;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 500. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 950).

In some embodiments, the guide RNA (gRNA) of the previous embodiment comprising a guide region and a conserved region, the conserved region comprising:
(a) a shortened repeat/anti-repeat region, wherein the shortened repeat/anti-repeat region lacks 18-22 nucleotides, wherein
   (i) nucleotides 37-48 and 53-64 are deleted relative to SEQ ID NO: 500; and
   (ii) nucleotide 36 is linked to nucleotide 65 by 6-10 nucleotides;
(b) a shortened hairpin 1 region, wherein the shortened hairpin 1 lacks 2 nucleotides relative to SEQ ID NO: 500, wherein nucleotides 86 and 91 are deleted or nucleotides 85 and 92 are deleted;
(c) a shortened hairpin 2 region, wherein the shortened hairpin 2 lacks 18 nucleotides, wherein nucleotides 113-121 and 126-134 are deleted relative to SEQ ID NO: 500; and
(d) wherein nucleotides 144-145 are deleted relative to SEQ ID NO: 500;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 500. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 950).

### A. Shortened Repeat/Anti-Repeat Region

In some embodiments, a gRNA described herein comprises a conserved region comprising a shortened repeat/anti-repeat region. In some embodiments, the repeat-anti-repeat region comprises a hairpin structure between a first portion and a second portion of the repeat-anti-repeat region, wherein the first portion and the second portion of the repeat-anti-repeat region together form a duplex portion.

In some embodiments, a gRNA described herein comprises a conserved region comprising a shortened upper stem region of the repeat/anti-repeat region. In some embodiments, the repeat/anti-repeat region comprises a loop (e.g., a tetraloop).

In some embodiments, the shortened repeat/anti-repeat region lacks 2-24 nucleotides. In some embodiments, (i) one or more of nucleotides 37-48 and 53-64 is deleted and optionally one or more of nucleotides 37-64 is substituted relative to SEQ ID NO: 500; and (ii) nucleotide 36 is linked to nucleotide 65 by at least 4 nucleotides.

In some embodiments, the shortened repeat/anti-repeat region lacks 2-24 nucleotides.

In some embodiments, the shortened repeat/anti-repeat region has a length of 28, 29, 30, 31, 32, 33, 34, 35, 36, 37, 38, 39, 40, 41, 42, 43, 44, 45, 46, 47, 48, 49, or 50 nucleotides.

In some embodiments, the shortened repeat/anti-repeat region lacks 12-24 nucleotides, optionally 18-24 nucleotides. In some embodiments, the shortened repeat/anti-repeat region has a length of 28, 29, 30, 31, 32, 33, 34, 35, 36, 37, 38, 39, or 40 nucleotides. In some embodiments, the shortened repeat/anti-repeat region has a length of 28, 29, 30, 31, 32, 33, or 34 nucleotides. In some embodiments, the shortened repeat/anti-repeat region has a length of 28 nucleotides. In some embodiments, the shortened repeat/anti-repeat region has a length of 29 nucleotides. In some embodiments, the shortened repeat/anti-repeat region has a length of 30 nucleotides. In some embodiments, the shortened repeat/anti-repeat region has a length of 31 nucleotides. In some embodiments, the shortened repeat/anti-repeat region has a length of 32 nucleotides. In some embodiments, the shortened repeat/anti-repeat region has a length of 33 nucleotides. In some embodiments, the shortened repeat/anti-repeat region has a length of 34 nucleotides.

In some embodiments, nucleotides 37-64 of SEQ ID NO: 500 form the upper stem, and one or more base pairs of the upper stem of the shortened repeat/anti-repeat region are deleted. In some embodiments, the upper stem of the shortened repeat/anti-repeat region comprises no more than one, two, three, or four base pairs. In some embodiments, all of positions 38-48 and all of positions 53-63 of the upper stem of the shortened repeat/anti-repeat region are deleted. In some embodiments, all of nucleotides 37-48 and 53-64 of the upper stem of the shortened repeat/anti-repeat region are deleted. As used herein, “base pairs” or “base paired nucleotides” or “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. As used herein, base pairs or base paired nucleotides also include base pairs generated by base stacking, e.g. nucleotides 25 and 76, 33 and 68, 34 and 67, and 37 and 64 in the repeat/anti-repeat region; and nucleotides 78 and 100, and 83 and 94 in the hairpin 1 region.

In some embodiments, one or more of positions 37-48 is deleted. In some embodiments, position 37 is deleted. In some embodiments, position 38 is deleted. In some embodiments, position 39 is deleted. In some embodiments, position 40 is deleted. In some embodiments, position 41 is deleted. In some embodiments, position 42 is deleted. In some embodiments, position 43 is deleted. In some embodiments, position 44 is deleted. In some embodiments, position 45 is deleted. In some embodiments, position 46 is deleted. In some embodiments, position 47 is deleted. In some embodiments, position 48 is deleted.

In some embodiments, one or more of positions 53-63 is deleted. In some embodiments, position 53 is deleted. In some embodiments, position 54 is deleted. In some embodiments, position 55 is deleted. In some embodiments, position 56 is deleted. In some embodiments, position 57 is deleted. In some embodiments, position 58 is deleted. In some embodiments, position 59 is deleted. In some embodiments, position 60 is deleted. In some embodiments, position 61 is deleted. In some embodiments, position 62 is deleted. In some embodiments, position 63 is deleted. In some embodiments, position 64 is deleted.

In some embodiments, the shortened repeat/anti-repeat region has a duplex portion 11 base paired nucleotides in length. In some embodiments, the shortened repeat/anti-repeat region has a single duplex portion.

In some embodiments, one or more of base paired nucleotides in the repeat/anti-repeat region is deleted. In some embodiments, one or more of based paired nucleotides chosen from positions 37 and 53, positions 38 and 54, position 39 and 55, positions 40 and 56, positions 41 and 57, positions 43 and 58, positions 43 and 59, positions 44 and 60, positions 45 and 61, positions 46 and 62, positions 47 and 63, and positions 48 and 64.

In some embodiments, the upper stem region of the repeat/anti-repeat region comprises 1-5 base pairs.

In some embodiments, the upper stem of the shortened repeat/anti-repeat region includes one or more substitution relative to SEQ ID NO: 500.

In some embodiments, one or more substitutions are conservative substitutions that maintain base pairing(s). For example, a G-C pair becomes a C-G pair or other natural or modified base pairing, or an A-U pair becomes a U-A pair or other natural or modified base pairing. In some embodiments, one or more substitutions are conservative substitutions that exchange positions of base paired nucleotides (e.g., a G-C pair becomes a C-G pair, or an A-U pair for becomes a U-A pair).

In some embodiments, one or more of nucleotides 49-52 is substituted relative to SEQ ID NO: 500. In some embodiments, the shortened repeat/anti-repeat region is unsubstituted.

In some embodiments, the shortened repeat/anti-repeat region has 12-22 modified nucleotides.

### B. Shortened Hairpin 1 Region

In some embodiments, a gRNA described herein comprises a conserved region comprising a shortened hairpin 1 region. In some embodiments, the hairpin 1 region comprises a hairpin structure between a first portion and a second portion of the hairpin 1 region, wherein the first portion and the second portion together form a duplex portion.

In some embodiments, a gRNA described herein comprises a conserved region comprising a shortened upper stem region of the hairpin 1 region. In some embodiments, the hairpin 1 comprises a loop (e.g., a tetraloop).

In some embodiments, the shortened hairpin 1 lacks 2-10 nucleotides. In some embodiments, the shortened hairpin 1 lacks 2-8 nucleotides. In some embodiments, the shortened hairpin 1 lacks 2-4 nucleotides. In some embodiments, the shortened hairpin lacks 2 nucleotides. In some embodiments, the shortened hairpin lacks 3 nucleotides. In some embodiments, the shortened hairpin lacks 4 nucleotides. In some embodiments, the shortened hairpin lacks 5 nucleotides. In some embodiments, the shortened hairpin lacks 6 nucleotides. In some embodiments, the shortened hairpin lacks 7 nucleotides. In some embodiments, the shortened hairpin lacks 8 nucleotides. In some embodiments, the shortened hairpin lacks 9 nucleotides. In some embodiments, the shortened hairpin lacks 10 nucleotides. In some embodiments, (i) one or more of nucleotides 82-86 and 91-95 is deleted and optionally one or more of positions 82-95 is substituted relative to SEQ ID NO: 500; and (ii) nucleotide 81 is linked to nucleotide 96 by at least 4 nucleotides.

In some embodiments, wherein the shortened hairpin 1 region lacks 2-10 nucleotides. In some embodiments, wherein the shortened hairpin 1 region has a length of 13, 14, 15, 16, 17, 18, 19, 20 or 21 nucleotides. In some embodiments, wherein the shortened hairpin 1 region has duplex portion 7-8 base paired nucleotides in length. As used herein, nucleotide 96 is not considered to interrupt the duplex portion of hairpin 1 when one or more of base pairs 82 and 95, 83 and 94, 85 and 93, and 86 and 92 are present.

In some embodiments, the shortened hairpin 1 region has a single duplex portion. In some embodiments, in the shortened hairpin 1 region, positions 78 and 100, and positions 83 and 94 have base stacking interactions and do not constitute a discontinuity in the duplex portion.

In some embodiments, one or two base pairs of the shortened hairpin 1 region are deleted. In some embodiments, the stem of the shortened hairpin 1 region comprises one, two, three, four, five, six, seven, or eight base pairs. In some embodiments, the stem of the shortened hairpin 1 region is seven or eight base paired nucleotides in length.

In some embodiments, one or more of positions 85-86 and one or more of nucleotides 91-92 of the shortened hairpin 1 region are deleted. In some embodiments, nucleotides 86 and 91 of the shortened hairpin 1 region are deleted. In some embodiments, nucleotides 85 and 92 of the shortened hairpin 1 region are deleted. In some embodiments, one or more of nucleotides 82-95 of the shortened hairpin 1 region is substituted relative to SEQ ID NO: 500. In some embodiments, one or more of nucleotides 87-91 is substituted relative to SEQ ID NO: 500.

In some embodiments, the shortened hairpin 1 region is unsubstituted. In some embodiments, wherein the shortened hairpin 1 region has 6-15 modified nucleotides.

### C. Shortened Hairpin 2 Region

In some embodiments, a gRNA described herein comprises a conserved region comprising a shortened hairpin 2 region. In some embodiments, the hairpin 2 region comprises a hairpin structure between a first portion and a second portion of the hairpin 2 region, wherein the first portion and the second portion together form a duplex portion.

In some embodiments, (c) a shortened hairpin 2 region, wherein the shortened hairpin 2 lacks 2-16 nucleotides. In some embodiments, (i) one or more of nucleotides 113-121 and 126-134 is deleted and optionally one or more of nucleotides 113-134 is substituted relative to SEQ ID NO: 500; and (ii) nucleotide 112 is linked to nucleotide 135 by at least 4 nucleotides.

In some embodiments, a conserved region of a gRNA described herein comprises a shortened upper stem region of the hairpin 2 region. In some embodiments, the hairpin 1 comprises a loop (e.g., a tetraloop). In some embodiments, the shortened hairpin 2 region lacks 2-16 nucleotides. In some embodiments, the shortened hairpin 2 region has a length of 26, 27, 28, 29, 30, 31, 32, 33, 34, 35, 36, 37, 38, 39, or 40 nucleotides. In some embodiments, the shortened hairpin 2 region has a length of 28, 29, 30, 31, 32, 33 or 34, nucleotides. In some embodiments, one or more of positions 113-121 and one or more of nucleotides 126-134 of the shortened hairpin 2 region are deleted.

The shortened hairpin 2 region comprises an unpaired region The unpaired region, nucleotides 106-108 and nucleotide 139 on the opposite strand, result in a discontinuity of the duplex portion within hairpin 2, providing two duplex portions, nucleotides 102-105 and 140-143, and nucleotides 109-112 and 135-138.

In some embodiments, the shortened hairpin 2 region has two duplex portions. In some embodiments, the shortened hairpin 2 region has one duplex portion of 4 base paired nucleotides in length. In some embodiments, the shortened hairpin 2 region has one duplex portion of 4-8 base paired nucleotides in length. In some embodiments, the shortened hairpin 2 region has one duplex portion of 4-6 base paired nucleotides in length. In some embodiments, the upper stem of the shortened hairpin 2 region comprises one, two, three, or four base pairs. In some embodiments, at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted. In some embodiments, all of positions 113-121 and 126-134 of the shortened hairpin 2 region are deleted.

In some embodiments wherein one or more of nucleotides 113-134 of the shortened hairpin 2 region is substituted relative to SEQ ID NO: 500. In some embodiments one or more of nucleotides 122-125 is substituted relative to SEQ ID NO: 500.

In some embodiments the shortened hairpin 2 region is unsubstituted. In some embodiments the shortened hairpin 2 region has 6-15 modified nucleotides.

### D. 3′ Tail

In some embodiments, the gRNA comprises a 3′ tail. In some embodiments, the 3′ tail is 1-20 nucleotides in length and is linked by a phosphodiester or a phosphorothioate linkage, to the 3′ end of the conserved region of a gRNA. In some embodiments, the 3′ tail comprises 1, 2, 3, 4, 5, 6, 7, 8, 9, or 10 nucleotides. In some embodiments, the 3′ tail comprises 1, 2, 3, 4, or 5 nucleotides. In some embodiments, the 3′ tail comprises 1 or 2 nucleotides.

In some embodiments, the 3′ tail has a length of 1-10 nucleotides, 1-5 nucleotides, 1-4 nucleotides, 1-3 nucleotides, and 1-2 nucleotides. In some embodiments, the 3′ tail comprises about 1, 2, 3, 4, 5, 6, 7, 8, 9, or 10 nucleotides. In some embodiments, the 3′ tail has a length of 1 nucleotide. In some embodiments, the 3′ tail has a length of 2 nucleotides. In some embodiments, the 3′ tail has a length of 3 nucleotides. In some embodiments, the 3′ tail has a length of 4 nucleotides. In some embodiments, the 3′ tail has a length of 1-2, nucleotides.

In some embodiments, the 3′ tail terminates with a nucleotide comprising a uracil or modified uracil. In some embodiments, the 3′ tail is 1 nucleotide in length. In some embodiments, the 3′ tail consists of a nucleotide comprising a uracil or modified uracil. In some embodiments, wherein the 3′ tail comprises a modification of any one or more of the nucleotides present in the 3′ tail. In further embodiments, wherein the modification of the 3′ tail is one or more of 2′-O-methyl (2′-OMe) modified nucleotide and a phosphorothioate (PS) linkage between nucleotides.

In some embodiments, the 3′ tail is fully modified.

In some embodiments, the 3′ nucleotide of the gRNA is a nucleotide comprising a uracil or modified uracil.

In some embodiments, one or more of nucleotides 144 and 145 are deleted relative to SEQ ID NO: 500. In some embodiments, both nucleotides 144 and 145 are deleted relative to SEQ ID NO: 500.

In some embodiments, the gRNA does not comprise a 3′ tail. In some embodiments, the 3′ end of the guide, that does not comprise a 3′ tail, terminates with a nucleotide comprising a uracil or modified uracil. In some embodiments, the 3′ tail consists of a nucleotide comprising a uracil or modified uracil. In some embodiments, the 3′ terminal nucleotide is a modified nucleotide. In some embodiments, the modification of the 3′ end is one or more of 2′-O-methyl (2′-OMe) modified nucleotide and a phosphorothioate (PS) linkage between nucleotide the terminal nucleotide and the penultimate nucleotide.

In some embodiments, the 3′ end, i.e., the end of hairpin 2 with no further tail or the end of the 3′ tail, comprises or further comprises one or more modifications, e.g., a phosphorothioate (PS) linkage between nucleotides, a 2′-OMe modified nucleotide, a 2′-O-moe modified nucleotide, a 2′-F modified nucleotide, an inverted abasic modified nucleotide, and a combination thereof. In some embodiments, the 3′ end comprises or further comprises one or more modifications, e.g., a phosphorothioate (PS) linkage between nucleotides, a 2′-OMe modified nucleotide, a 2′-F modified nucleotide, and a combination thereof. In some embodiments, the 3′ end comprises phosphorothioate (PS) linkage between nucleotides 141 and 142, and 142 and 143; a 2′-OMe modified nucleotide at each of positions 142 and 143.

In some embodiments, the 3′ end, i.e., the end of hairpin 2 with no further tail or the end of the 3′ tail, comprises or further comprises one or more phosphorothioate (PS) linkages between nucleotides. In some embodiments, the 3′ end comprises or further comprises one or more 2′-OMe modified nucleotides. In some embodiments, the 3′ end comprises or further comprises one or more 2′-O-moe modified nucleotides. In some embodiments, the 3′ end comprises or further comprises one or more 2′-F modified nucleotide. In some embodiments, the 3′ end comprises or further comprises one or more an inverted abasic modified nucleotides. In some embodiments, the 3′ end comprises or further comprises one or more protective end modifications. In some embodiments, the 3′ end comprises or further comprises a combination of one or more of a phosphorothioate (PS) linkage between nucleotides, a 2′-OMe modified nucleotide, a 2′-O-moe modified nucleotide, a 2′-F modified nucleotide, and an inverted abasic modified nucleotide.

### E. Guide Sequence

In some embodiments, the gRNA further comprises a guide sequence. In some embodiments, the guide sequence comprises 20, 21, 22, 23, 24, or 25 nucleotides, optionally 22, 23, 24, or 25 nucleotides 5′ to the most 5′ nucleotide of the repeat/anti-repeat region. In some embodiments, the guide sequence comprises 22, 23, 24, 25, or more nucleotides. In some embodiments, the guide sequence has a has a length of 24 nucleotides. In some embodiments, the guide sequence has a length of 23 nucleotides. In some embodiments, the guide sequence has a length of 22 nucleotides. In some embodiments, the guide sequence has a length of 21 nucleotides. In some embodiments, the guide sequence has a length of 20 nucleotides.

In some embodiments, the guide region has (i) an insertion of one nucleotide or a deletion of 1-4 nucleotides within positions 1-24 relative to SEQ ID NO: 500, or (ii) a length of 24 nucleotides.

In some embodiments, the selection of the guide sequence is determined based on target sequences within the gene of interest for editing. For example, in some embodiments, the gRNA comprises a guide sequence that is complementary to target sequences of a gene of interest.

In some embodiments, the target sequence in the gene of interest may be complementary to the guide sequence of the gRNA. In some embodiments, the degree of complementarity or identity between a guide sequence of a gRNA and its corresponding target sequence in the gene of interest may be about 90%, 95%, or 100%. In some embodiments, the guide region of a gRNA and the target region of a gene of interest may be 100% complementary or identical. In other embodiments, the guide sequence of a gRNA and the target sequence of a gene of interest may contain at least one mismatch. For example, the guide sequence of a gRNA and the target sequence of a gene of interest may contain 1, optionally 2, or 3 mismatches, where the total length of the target sequence is at least about 22, 23, 24, or more nucleotides. In some embodiments, the guide sequence of a gRNA and the target region of a gene of interest may contain 1, optionally 2, or 3 mismatches where the guide sequence comprises about 24 nucleotides. In certain embodiments, the guide sequence contains no mismatches, i.e., is fully complementary, to the target sequence. The 5′ terminus may comprise nucleotides that are not considered guide regions (i.e., do not function to direct a Cas9 protein to a target nucleic acid).

II. Modified Guide RNA (gRNA)

Guide RNAs comprising modifications at various positions are disclosed herein. In some embodiments, a position of a gRNA that comprises a modification is modified with any one or more of the following types of modifications. The term “modified gRNA” generally refers to a gRNA having a modification to the chemical structure of one or more of the bases, the sugar, the phosphodiester linkage or backbone portions, including nucleotide phosphates, all as detailed and exemplified herein.

In some embodiments, the guide region of the gRNA comprises at least one modified nucleotide.

In some embodiments, the guide region of the gRNA comprises at least two modified nucleotides, optionally at least four modified nucleotides, wherein each modification, independently, optionally comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the guide region of the gRNA comprises 1, 2, 3, 4, 5, 6, 7, 8, 9, 10, 11, or 12 modified nucleotides. In some embodiments, the guide region of the gRNA comprises 1, 2, or 3 modified nucleotides. In some embodiments, the guide region of the gRNA comprises 4, 5, 6, 7, 8, 9, 10, 11, or 12 modified nucleotides. In some embodiments, the guide region of the gRNA comprises 6, 7, 8, 9, 10, 11, or 12 modified nucleotides.

In some embodiments, the gRNA comprises a 5′ end modification. In some embodiments, the gRNA further comprises a 3′ end modification.

In some embodiments, the guide region does not comprise a modified nucleotide 3′ of the first three nucleotides of the guide region.

In some embodiments, the guide region does not comprise a modified nucleotide.

In some embodiments, wherein the gRNA comprises a 3′ end modification. In some embodiments, the gRNA comprises a modification in the upper stem region of the repeat/anti-repeat region. In some embodiments, the gRNA comprises a modification in the hairpin 1 region. In some embodiments, the gRNA comprises a modification in the hairpin 2 region. In some embodiments, the gRNA comprises a 3′ end modification, and comprising a modification in the upper stem region of the repeat/anti-repeat region. In some embodiments, the gRNA comprises a 3′ end modification, and a modification in the hairpin 1 region. In some embodiments, the gRNA comprises a 3′ end modification, and a modification in the hairpin 2 region. In some embodiments, the gRNA comprises a 5′ end modification, and comprising a modification in the upper stem region of the repeat/anti-repeat region. In some embodiments, the gRNA comprises a 5′ end modification, and a modification in the hairpin 1 region. In some embodiments, the gRNA comprises a 5′ end modification, and a modification in the hairpin 2 region. In some embodiments, the gRNA comprises a 5′ end modification, a modification in the upper stem region of the repeat/anti-repeat region, and a 3′ end modification. In some embodiments, the gRNA comprises a 5′ end modification, a modification in the hairpin 1 region, and a 3′ end modification. In some embodiments, the gRNA comprises a 5′ end modification, a modification in the hairpin 1 region, a modification in the hairpin 2 region, and a 3′ end modification. In some embodiments, the gRNA comprises a 5′ end modification, a modification in the repeat/anti-repeat region, a modification in the hairpin 1 region, a modification in the hairpin 2 region, and a 3′ end modification.

In some embodiments, the gRNA does not comprise a modification at position 76. In some embodiments, the gRNA does not comprise a PS modification at position 76, i.e., a PS modification between nucleotides 76 and 77.

In some embodiments, the gRNA comprises one or more, i.e., 1, 2, 3, or 4 modifications at positions 106-109. In some embodiments, the gRNA comprises modifications at positions 106-109. In some embodiments, the modification comprises a 2′-O-methyl (2′-O-Me) modified nucleotide.

In some embodiments, the gRNA comprises a 2′-O-methyl (2′-O-Me) modified nucleotide. In some embodiments, the gRNA comprises a 2′-O-(2-methoxyethyl) (2′-O-moe) modified nucleotide. In some embodiments, the gRNA comprises a 2′-fluoro (2′-F) modified nucleotide. In some embodiments, the gRNA comprises a phosphorothioate (PS) bond between nucleotides.

In some embodiments, the gRNA comprises a 5′ end modification, a 3′ end modification, or 5′ and 3′ end modification, such as a protective end modification. In some embodiments, the 5′ end modification comprises a phosphorothioate (PS) bond between nucleotides. In some embodiments, the 5′ end modification comprises a 2′-O-methyl (2′-O-Me), 2′-O-(2-methoxyethyl) (2′-O-moe), or 2′-fluoro (2′-F) modified nucleotide. In some embodiments, the 5′ end modification comprises at least one phosphorothioate (PS) bond and one or more of a 2′-O-methyl (2′-O-Me), 2′-O-(2-methoxyethyl) (2′-O-moe), or 2′-fluoro (2′-F) modified nucleotide. The end modification may comprise a phosphorothioate (PS), 2′-O-methyl (2′-O-Me), 2′-O-(2-methoxyethyl) (2′-O-moe), or 2′-fluoro (2′-F) modification. Equivalent end modifications are also encompassed by embodiments described herein. In some embodiments, the gRNA comprises an end modification in combination with a modification of one or more regions of the gRNA.

Exemplary patterns of modifications are shown in Tables 1-2. In certain embodiments, exemplary modifications include patterns of modifications shown in Tables 1-2 in which 3′ tails, when present, are deleted. Additional exemplary patterns are discussed below.

### Types of Chemical Modifications Described Herein

### 2′-O-Methyl Modifications

Modified sugars are believed to control the puckering of nucleotide sugar rings, a physical property that influences oligonucleotide binding affinity for complementary strands, duplex formation, and interaction with nucleases. Substitutions on sugar rings can therefore alter the conformation and puckering of these sugars. For example, 2′-O-methyl (2′-OMe) modifications can increase binding affinity and nuclease stability of oligonucleotides, though as shown in the Examples, the effect of any modification at a given position in an oligonucleotide needs to be empirically determined.

The terms “mA,” “mC,” “mU,” or “mG” may be used to denote a nucleotide that has been modified with 2′-OMe.

A ribonucleotide and a modified 2′-O-methyl ribonucleotide can be depicted as follows:

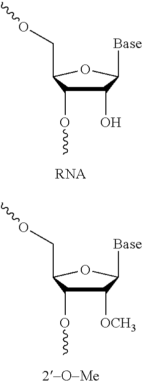

### 2′-O-(2-Methoxyethyl) Modifications

In some embodiments, the modification may be 2′-O-(2-methoxyethyl) (2′-O-moe). A modified 2′-O-moe ribonucleotide can be depicted as follows:

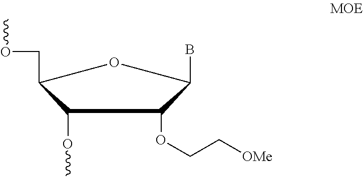

The terms “moeA,” “moeC,” “moeU,” or “moeG” may be used to denote a nucleotide that has been modified with 2′-O-moe.

### 2′-Fluoro Modifications

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fJ,” or “fG” may be used to denote a nucleotide that has been substituted with 2′-F.

A ribonucleotide without and with a 2′-F substitution can be depicted as follows:

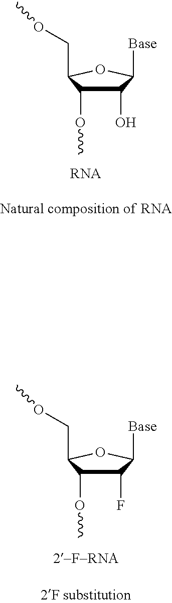

### Phosphorothioate Modifications

A phosphorothioate (PS) linkage or bond refers to a bond where a sulfur is substituted for one nonbridging phosphate oxygen in a phosphodiester linkage, for example between nucleotides. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond. Throughout this application, PS modifications are grouped with the nucleotide whose 3′ carbon is bonded to the phosphorothioate; thus, indicating that a PS modification is at position 1 means that the phosphorothioate is bonded to the 3′ carbon of nucleotide 1 and the 5′ carbon of nucleotide 2.

In this application, the terms “mA*,” “mC*,” “mU*,” or “mG*” may be used to denote a nucleotide that has been substituted with 2′-OMe and that is linked to the next (e.g., 3′) nucleotide with a PS linkage, which may sometimes be referred to as a “PS bond.” Similarly, the terms “fA*,” “fC*,” “fU*,” or “fG*” may be used to denote a nucleotide that has been substituted with 2′-F and that is linked to the next (e.g., 3′) nucleotide with a PS linkage. Equivalents of a PS linkage or bond are encompassed by embodiments described herein.

The diagram below shows the substitution of S- for a nonbridging phosphate oxygen, generating a PS linkage in lieu of a phosphodiester linkage:

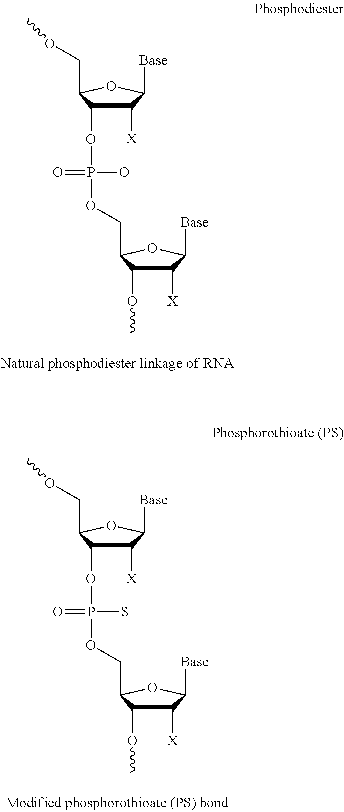

### Inverted Abasic Modifications

Abasic nucleotides refer to those which lack nitrogenous bases. The figure below depicts an oligonucleotide with an abasic (in this case, shown as apurinic; an abasic site could also be an apyrimidinic site, wherein the description of the abasic site is typically in reference to Watson-Crick base pairing—e.g., an apurinic site refers to a site that lacks a nitrogenous base and would typically base pair with a pyrimidinic site) site that lacks a base, wherein the base may be substituted by another moiety at the 1′ position of the furan ring (e.g., a hydroxyl group, as shown below, to form a ribose or deoxyribose site, as shown below, or a hydrogen):

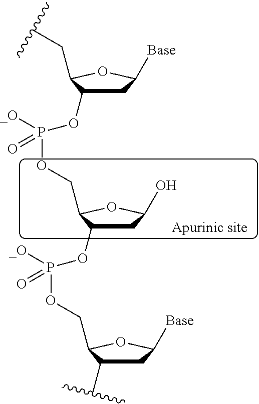

Inverted bases refer to those with linkages that are inverted from the normal 5′ to 3′ linkage (i.e., either a 5′ to 5′ linkage or a 3′ to 3′ linkage). For example:

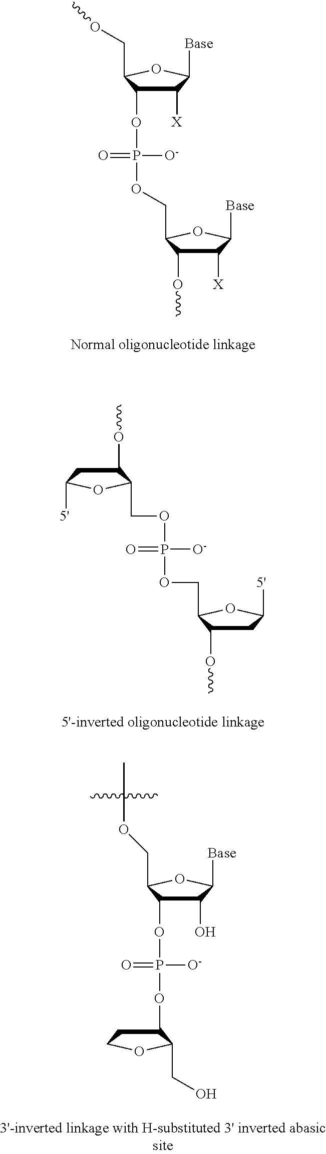

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap. In this application, the terms “invd” indicates an inverted abasic nucleotide linkage.

### Deoxyribonucleotides

A deoxyribonucleotide (in which the sugar comprises a 2′-deoxy position) is considered a modification in the context of a gRNA, in that the nucleotide is modified relative to standard RNA by the substitution of a proton for a hydroxyl at the 2′ position. Unless otherwise indicated, a deoxyribonucleotide modification at a position that is U in an unmodified RNA can also comprise replacement of the U nucleobase with a T.

### Bicyclic Ribose Analog

Exemplary bicyclic ribose analogs include locked nucleic acid (LNA), ENA, bridged nucleic acid (BNA), or another LNA-like modifications. In some instances, a bicyclic ribose analog has 2′ and 4′ positions connected through a linker. The linker can be of the formula —X—(CH₂)ₙ— where n is 1 or 2; X is O, NR, or S; and R is H or C₁₋₃ alkyl, e.g., methyl. Examples of bicyclic ribose analogs include LNAs comprising a 2′-O—CH₂-4′ bicyclic structure (oxy-LNA) (see WO 98/39352 and WO 99/14226); 2′-NH—CH₂-4′ or 2′-N(CH₃)—CH₂-4′ (amino-LNAs) (Singh et al., J. Org. Chem. 63:10035-10039 (1998); Singh et al., J. Org. Chem. 63:6078-6079 (1998)); and 2′-S—CH₂-4′ (thio-LNA) (Singh et al., J. Org. Chem. 63:6078-6079 (1998); Kumar et al., Biorg. Med. Chem. Lett. 8:2219-2222 (1998)).

### ENA

An ENA modification refers to a nucleotide comprising a 2′-O,4′-C-ethylene modification. An exemplary structure of an ENA nucleotide is shown below, in which wavy lines indicate connections to the adjacent nucleotides (or terminal positions as the case may be, with the understanding that if the 3′ terminal nucleotide is an ENA nucleotide, the 3′ position may comprise a hydroxyl rather than phosphate). For further discussion of ENA nucleotides, see, e.g., Koizumi et al., Nucleic Acids Res. 31: 3267-3273 (2003).

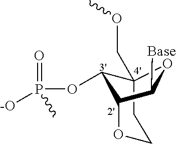

### UNA

A UNA or unlocked nucleic acid modification refers to a nucleotide comprising a 2′,3′-seco-RNA modification, in which the 2′ and 3′ carbons are not bonded directly to each other. An exemplary structure of a UNA nucleotide is shown below, in which wavy lines indicate connections to the adjacent phosphates or modifications replacing phosphates (or terminal positions as the case may be). For further discussion of UNA nucleotides, see, e.g., Snead et al., Molecular Therapy 2: e103, doi:10.1038/mtna.2013.36 (2013).

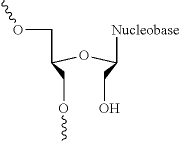

### Base Modifications

A base modification is any modification that alters the structure of a nucleobase or its bond to the backbone, including isomerization (as in pseudouridine). In some embodiments, a base modification includes inosine. In some embodiments, a modification comprises a base modification that reduces RNA endonuclease activity, e.g., by interfering with recognition of a cleavage site by an RNase or by stabilizing an RNA structure (e.g., secondary structure) that decreases accessibility of a cleavage site to an RNase. Exemplary base modifications that can stabilize RNA structures are pseudouridine and 5-methylcytosine. See Peacock et al., J Org Chem. 76: 7295-7300 (2011). In some embodiments, a base modification can increase or decrease the melting temperature (Tm) of a nucleic acid, e.g., by increasing the hydrogen bonding in a Watson-Crick base pair, forming non-canonical base pair, or creating a mismatched base pair.

The above modifications and their equivalents are included within the scope of the embodiments described herein.

### 3′ End Modifications

In some embodiments, the terminal (i.e., last) 1, 2, 3, or 4, optionally 5, 6, or 7 nucleotides in the 3′ end are modified. Throughout, this modification may be referred to as a “3′ end modification”. In some embodiments, the terminal (i.e., last) 1, 2, 3, or 4, optionally 5, 6, or 7 nucleotides in the 3′ end comprise more than one modification. In some embodiments, at least one of the terminal (i.e., last) 1, 2, 3, or 4, optionally 5, 6, or 7 nucleotides in the 3′ end are modified. In some embodiments, at least two of the terminal (i.e., last) 1, 2, 3, or 4, optionally 5, 6, or 7 nucleotides in the 3′ end are modified. In some embodiments, at least three of the terminal (i.e., last) 1, 2, 3, or 4, optionally 5, 6, or 7 nucleotides in the 3′ end are modified. In some embodiments, the modification comprises a PS linkage. In some embodiments, the modification to the 3′ end is a 3′ protective end modification. In some embodiments, the 3′ end modification comprises a 3′ protective end modification.

In some embodiments, the 3′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide, optionally wherein the gRNA comprises at least two 3′ end modifications independently selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide.

In some embodiments, the 3′ end modification comprises or further comprises a 2′-O-methyl (2′-O-Me) modified nucleotide.

In some embodiments, the 3′ end modification comprises or further comprises a 2′-fluoro (2′-F) modified nucleotide.

In some embodiments, the 3′ end modification comprises or further comprises a phosphorothioate (PS) linkage between nucleotides.

In some embodiments, the 3′ end modification comprises or further comprises an inverted abasic modified nucleotide.

In some embodiments, the 3′ end modification comprises or further comprises a 2′-O-methyl (2′-O-Me) modified nucleotide and a phosphorothioate (PS) linkage between nucleotides.

In some embodiments, the 3′ end modification comprises or further comprises a modification of any one or more of the last 1, 2, 3, or 4, optionally 5, 6, or 7 nucleotides. In some embodiments, the 3′ end modification comprises or further comprises one modified nucleotide. In some embodiments, the 3′ end modification comprises or further comprises two modified nucleotides. In some embodiments, the 3′ end modification comprises or further comprises three modified nucleotides. In some embodiments, the 3′ end modification comprises or further comprises four modified nucleotides. In some embodiments, the 3′ end modification comprises or further comprises five modified nucleotides. In some embodiments, the 3′ end modification comprises or further comprises six modified nucleotides. In some embodiments, the 3′ end modification comprises or further comprises seven modified nucleotides.

In some embodiments, the 3′ end modification comprises or further comprises a modification of 1-7 or 14 nucleotides.

In some embodiments, the 3′ end modification comprises or further comprises modifications of 1, 2, 3, or 4, optionally 5, 6, or 7 nucleotides at the 3′ end of the gRNA.

In some embodiments, the 3′ end modification comprises or further comprises modifications of about 1-3, 1-4, or 1-5 nucleotides at the 3′ end of the gRNA.

In some embodiments, the 3′ end modification comprises or further comprises any one or more of the following: a phosphorothioate (PS) linkage between nucleotides, a 2′-O-Me modified nucleotide, a 2′-O-moe modified nucleotide, a 2′-F modified nucleotide, an inverted abasic modified nucleotide, and a combination thereof.

In some embodiments, the 3′ end modification comprises or further comprises 1, 2, 3, or 4, optionally 5, 6, or 7 PS linkages between nucleotides.

In some embodiments, the 3′ end modification comprises or further comprises at least one 2′-O-Me, 2′-O-moe, inverted abasic, or 2′-F modified nucleotide. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides.

In some embodiments, the 3′ end modification comprises or further comprises PS linkages between any one or more of the last four nucleotides. In some embodiments, the 3′ end modification comprises or further comprises PS linkages between any one or more of the last three nucleotides. In some embodiments, the 3′ end modification comprises or further comprises PS linkages between any one or more of the last 2, 3, or 4, optionally 5, 6, or 7 nucleotides.

In some embodiments, the 3′ end modification comprises or further comprises a modification of one or more of the last 1-4, optionally 1-7 nucleotides, wherein the modification is a PS linkage, inverted abasic nucleotide, 2′-OMe, 2′-O-moe, 2′-F, or combinations thereof.

In some embodiments, the 3′ end modification comprises or further comprises a modification to the last nucleotide with 2′-OMe, 2′-O-moe, 2′-F, or combinations thereof, and an optionally one or two PS linkages to the next nucleotide or the first nucleotide of the 3′ end.

In some embodiments, the 3′ end modification comprises or further comprises a modification to the last or second to last nucleotide with 2′-OMe, 2′-O-moe, 2′-F, or combinations thereof, and optionally one or more PS linkages.

In some embodiments, the 3′ end modification comprises or further comprises a modification to the last, second to last, or third to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, or fourth to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, fourth to last, or fifth to last nucleotides with 2′-OMe, 2′-O-moe, 2′-F, or combinations thereof, and optionally one or more PS linkages.

In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified. In some embodiments, the 3′ tail comprises 1-4 nucleotides, optionally 1-2 nucleotides.

In some embodiments, a gRNA is provided comprising a 3′ end modification, wherein the 3′ end modification comprises the 3′ end modification as shown in any one of SEQ ID NOs: In some embodiments, a gRNA is provided comprising a 5′ end modification, wherein the 5′ end modification comprises a 5′ end modification as shown in any one of SEQ ID NOs: 4-9 and 301-494. In some embodiments, a gRNA is provided comprising a 3′ protective end modification.

In some embodiments, the gRNA comprises a 5′ end modification and a 3′ end modification.

### 5′ End Modifications

In some embodiments, the 5′ end is modified, for example, the first 1, 2, 3, or 4, optionally 5, 6, or 7 nucleotides of the gRNA are modified. Throughout, this modification may be referred to as a “5′ end modification”. In some embodiments, the first 1, 2, 3, or 4, optionally 5, 6, or 7 nucleotides of the 5′ end comprise more than one modification. In some embodiments, at least one of the terminal (i.e., first) 1, 2, 3, or 4, optionally 5, 6, or 7 nucleotides at the 5′ end are modified. In some embodiments, at least two of the terminal 1, 2, 3, or 4, optionally 5, 6, or 7 nucleotides at the 5′ end are modified. In some embodiments, at least three of the terminal 1, 2, 3, or 4, optionally 5, 6, or 7 nucleotides at the 5′ end are modified. In some embodiments, the 5′ end modification is a 5′ protective end modification.

In some embodiments, both the 5′ and 3′ ends of the gRNA are modified. In some embodiments, only the 5′ end of the gRNA is modified. In some embodiments, only the 3′ end of the conserved region of a gRNA is modified.

In some embodiments, the gRNA comprises modifications at 1, 2, 3, or 4, optionally 5, 6, or 7 of the first 4 nucleotides, optionally the first 7 nucleotides at a 5′ terminus region of the gRNA. In some embodiments, the gRNA comprises modifications at 1, 2, 3, or 4, optionally 5, 6, or 7 of the 4 terminal nucleotides, optionally 7 terminal nucleotides at a 3′ end. In some embodiments, 1, 2, 3, or 4 of the first 4 nucleotides at the 5′ end, or 1, 2, 3, or 4 of the terminal 4 nucleotides at the 3′ end are modified. In some embodiments, 2, 3, or 4 of the first 4 nucleotides at the 5′ end are linked with phosphorothioate (PS) bonds.

In some embodiments, the modification to the 5′ terminus or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, the gRNA comprises one or more phosphorothioate (PS) linkages between the first one, two, three, four, five, six, or seven nucleotides at the 5′ terminus. In some embodiments, the gRNA comprises one or more PS linkages between the last one, two, three, or four, optionally five, six, or seven nucleotides at the 3′ terminus. In some embodiments, the gRNA comprises one or more PS linkages between both the last one, two, three, or four, optionally five, six, or seven nucleotides at the 3′ terminus and the first one, two, three, or four, optionally five, six, or seven nucleotides from the 5′ end of the 5′ terminus. In some embodiments, in addition to PS linkages, the 5′ and 3′ terminal nucleotides may comprise 2′-O-Me, 2′-O-moe, or 2′-F modified nucleotides.

In some embodiments, the gRNA comprises a 5′ end modification, e.g., wherein the first nucleotide of the guide region is modified. In some embodiments, the gRNA comprises a 5′ end modification, wherein the first nucleotide of the guide region comprises a 5′ protective end modification.

In some embodiments, the 5′ end modification comprises a modified nucleotide selected from a 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, optionally wherein the gRNA comprises at least two 5′ end modifications independently selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide.

In some embodiments, the 5′ end modification comprises or further comprises a 2′-O-methyl (2′-O-Me) modified nucleotide.

In some embodiments, the 5′ end modification comprises or further comprises a 2′-fluoro (2′-F) modified nucleotide.

In some embodiments, the 5′ end modification comprises or further comprises a phosphorothioate (PS) linkage between nucleotides.

In some embodiments, the 5′ end modification comprises or further comprises an inverted abasic modified nucleotide.

In some embodiments, the 5′ end modification comprises or further comprises a 2′-O-methyl (2′-O-Me) modified nucleotide and a phosphorothioate (PS) linkage between nucleotides.

In some embodiments, the 5′ end modification comprises or further comprises a modification of any one or more of nucleotides 1-4, optionally 1-7 of the guide region of a gRNA. In some embodiments, the 5′ end modification comprises or further comprises one modified nucleotide. In some embodiments, the 5′ end modification comprises or further comprises two modified nucleotides. In some embodiments, the 5′ end modification comprises or further comprises three modified nucleotides. In some embodiments, the 5′ end modification comprises or further comprises four modified nucleotides. In some embodiments, the 5′ end modification comprises or further comprises five modified nucleotides. In some embodiments, the 5′ end modification comprises or further comprises six modified nucleotides. In some embodiments, the 5′ end modification comprises or further comprises seven modified nucleotides.

In some embodiments, the 5′ end modification comprises or further comprises a modification of 1-7, 1-5, 1-4, 1-3, or 1-2 nucleotides.

In some embodiments, the 5′ end modification comprises or further comprises modifications of 1, 2, 3, or 4, optionally 5, 6, or 7 nucleotides from the 5′ end. In some embodiments, the 5′ end modification comprises or further comprises modifications of about 1-3, 1-4, 1-5, 1-6, or 1-7 nucleotides from the 5′ end.

In some embodiments, the 5′ end modification comprises or further comprises modifications at the first nucleotide at the 5′ end of the gRNA. In some embodiments, the 5′ end modification comprises or further comprises modifications at the first and second nucleotide from the 5′ end of the gRNA. In some embodiments, the 5′ end modification comprises or further comprises modifications at the first, second, and third nucleotide from the 5′ end of the gRNA. In some embodiments, the 5′ end modification comprises or further comprises modifications at the first, second, third, and fourth nucleotide from the 5′ end of the gRNA. In some embodiments, the 5′ end modification comprises or further comprises modifications at the first, second, third, fourth, and fifth nucleotide from the 5′ end of the gRNA. In some embodiments, the 5′ end modification comprises or further comprises modifications at the first, second, third, fourth, fifth, and sixth nucleotide from the 5′ end of the gRNA. In some embodiments, the 5′ end modification comprises or further comprises modifications at the first, second, third, fourth, fifth, sixth, and seventh nucleotide from the 5′ end of the gRNA.

In some embodiments, the 5′ end modification comprises or further comprises a phosphorothioate (PS) linkage between nucleotides, or a 2′-O-Me modified nucleotide, or a 2′-O-moe modified nucleotide, or a 2′-F modified nucleotide, or an inverted abasic modified nucleotide, or combinations thereof.

In some embodiments, the 5′ end modification comprises or further comprises 1, 2, 3, 4, 5, 6, or 7 PS linkages between nucleotides. In some embodiments, the 5′ end modification comprises or further comprises about 1-2, 1-3, 1-4, 1-5, 1-6, or 1-7 PS linkages between nucleotides.

In some embodiments, the 5′ end modification comprises or further comprises at least one PS linkage, wherein if there is one PS linkage, the linkage is between nucleotides 1 and 2 of the guide region.

In some embodiments, the 5′ end modification comprises or further comprises at least two PS linkages, and the linkages are between nucleotides 1 and 2, and 2 and 3 of the guide region.

In some embodiments, the 5′ end modification comprises or further comprises PS linkages between any one or more of nucleotides 1 and 2, 2 and 3, and 3 and 4 of the guide region.

In some embodiments, the 5′ end modification comprises or further comprises PS linkages between any one or more of nucleotides 1 and 2, 2 and 3, 3 and 4, and 4 and 5 of the guide region.

In some embodiments, the 5′ end modification comprises or further comprises PS linkages between any one or more of nucleotides 1 and 2, 2 and 3, 3 and 4, 4 and 5, and 5 and 6 of the guide region.

In some embodiments, the 5′ end modification comprises or further comprises PS linkages between any one or more of nucleotides 1 and 2, 2 and 3, 3 and 4, 4 and 5, 5 and 6, and 7 and 8 of the guide region.

In some embodiments, the 5′ end modification comprises or further comprises a modification of one or more of nucleotides 1-7 of the guide region, wherein the modification is a PS linkage, inverted abasic nucleotide, 2′-O-Me, 2′-O-moe, 2′-F, or combinations thereof.

In some embodiments, the 5′ end modification comprises or further comprises a modification to the first nucleotide of the guide region with 2′-O-Me, 2′-O-moe, 2′-F, or combinations thereof, and an optional PS linkage to the next nucleotide;

In some embodiments, the 5′ end modification comprises or further comprises a modification to the first or second nucleotide of the guide region with 2′-O-Me, 2′-O-moe, 2′-F, or combinations thereof, and optionally one or more PS linkages between the first and second nucleotide or between the second and third nucleotide.

In some embodiments, the 5′ end modification comprises or further comprises a modification to the first, second, or third nucleotides of the variable region with 2′-O-Me, 2′-O-moe, 2′-F, or combinations thereof, and optionally one or more PS linkages between the first and second nucleotide, between the second and third nucleotide, or between the third and the fourth nucleotide.

In some embodiments, the 5′ end modification comprises or further comprises a modification to the first, second, third, or fourth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, or between the fourth and the fifth nucleotide.

In some embodiments, the 5′ end modification comprises or further comprises a modification to the first, second, third, fourth, or fifth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, between the fourth and the fifth nucleotide, or between the fifth and the sixth nucleotide.

In some embodiments, a gRNA is provided comprising a 5′ end modification, wherein the 5′ end modification comprises a 5′ end modification as shown in any one of SEQ ID NOs: 4-9 and 301-494, 931-946.

In some embodiments, the sgRNA comprises a 5′ end modification comprising a 5′ protective end modification. In some embodiments, a gRNA is provided comprising a 5′ end modification, wherein the 5′ end modification comprises 2′-OMe modified nucleotides at nucleotides 1, 2, and 3 of the guide region.

In some embodiments, a gRNA is provided comprising a 5′ end modification, wherein the 5′ end modification comprises 2′-OMe modified nucleotides at nucleotides 1, 2, and 3 of the guide region and PS linkages between nucleotides 1 and 2, 2 and 3, and 3 and 4 of the guide region.

In some embodiments, a gRNA is provided comprising a 5′ end modification, wherein the 5′ end modification comprises 2′-OMe modified nucleotides at nucleotides 1, 2, 3, 4, and 5 of the guide region.

In some embodiments, a gRNA is provided comprising a 5′ end modification, wherein the 5′ end modification comprises 2′-OMe modified nucleotides at nucleotides 1, 2, 3, 4, and 5 of the guide region and PS linkages between nucleotides 1 and 2, 2 and 3, and 3 and 4 of the guide region.

In some embodiments, a gRNA is provided comprising a 5′ end modification and a 3′ end modification. In some embodiments, the gRNA comprises modified nucleotides at the 5′ and 3′ terminus, and modified nucleotides in one or more other regions described in Table 3.

In some embodiments, the sgRNA comprises modified nucleotides that are not at the 5′ or 3′ ends. Exemplary patterns of modifications are described below and in Table 1.

### Repeat/Anti-Repeat Modifications

In some embodiments, a gRNA is provided comprising a repeat/anti-repeat region modification, wherein the repeat/anti-repeat region modification comprises a modification to any one or more of nucleotides 25-76 in the upper stem region.

In some embodiments, a gRNA is provided comprising a repeat/anti-repeat region modification, wherein the upper stem modification comprises a modification of at least 1, 2, 3, 4, 5, 6, 7, 8, 9, 10, 11, 12, 13, 14, 15, 16, 17, or 18 nucleotides in the repeat/anti-repeat region.

In some embodiments, a gRNA is provided comprising an upper stem modification, wherein the upper stem modification comprises a modification of about 1-18, 1-16, 1-15, 5-18, 5-15, 8-18, 8-15, 10-18, 10-15, or 12-15 nucleotides in the repeat/anti-repeat region.

In some embodiments, a gRNA is provided comprising a repeat/anti-repeat modification, wherein the repeat/anti-repeat modification comprises a 2′-OMe modified nucleotide. In some embodiments, a gRNA is provided comprising a repeat/anti-repeat modification, wherein the repeat/anti-repeat modification comprises a 2′-O-moe modified nucleotide. In some embodiments, a gRNA is provided comprising a repeat/anti-repeat modification, wherein the repeat/anti-repeat modification comprises a 2′-F modified nucleotide.

In some embodiments, a gRNA is provided comprising a repeat/anti-repeat modification, wherein the repeat/anti-repeat modification comprises a 2′-OMe modified nucleotide, a 2′-O-moe modified nucleotide, a 2′-F modified nucleotide, or combinations thereof.

In some embodiments, the sgRNA comprises a repeat/anti-repeat modification as shown in any one of the sequences in Table 1 or 2. In some embodiments, the gRNA does not comprise a modification at position 76 in the repeat/anti-repeat region. In some embodiments, the gRNA does not comprise a PS modification at position 76.

In some embodiments, such a repeat/anti-repeat modification is combined with a 5′ protective end modification, e.g. as shown for the corresponding sequence in Table 1 or 2. In some embodiments, such a repeat/anti-repeat modification is combined with a 3′ protective end modification, e.g. as shown for the corresponding sequence in Table 1 or 2. In some embodiments, such a repeat/anti-repeat modification is combined with 5′ and 3′ end modifications as shown for the corresponding sequence in Table 1 or 2.

In some embodiments, the gRNA comprises a 5′ end modification and a repeat/anti-repeat modification. In some embodiments, the gRNA comprises a 3′ end modification and a repeat/anti-repeat modification. In some embodiments, the gRNA comprises a 5′ end modification, a 3′ end modification and a repeat/anti-repeat modification.

### Hairpin Modifications

In some embodiments, the gRNA comprises a modification in the hairpin region (e.g., hairpin 1 region or hairpin 2 region). In some embodiments, the hairpin region modification comprises at least one modified nucleotide selected from a 2′-O-methyl (2′-OMe) modified nucleotide, a 2′-fluoro (2′-F) modified nucleotide, or combinations thereof.

In some embodiments, the hairpin region modification is in the hairpin 1 region. In some embodiments, the hairpin region modification is in the hairpin 2 region. In some embodiments, modifications are within the hairpin 1 and hairpin 2 regions, optionally wherein a nucleotide between hairpin 1 and 2 is also modified.

In some embodiments, the hairpin modification comprises or further comprises a 2′-O-methyl (2′-OMe) modified nucleotide.

In some embodiments, the hairpin modification comprises or further comprises a 2′-fluoro (2′-F) modified nucleotide.

In some embodiments, the hairpin region modification comprises at least one modified nucleotide selected from a 2′H modified nucleotide (DNA), PS modified nucleotide, a 2′-O-methyl (2′-O-Me) modified nucleotide, a 2′-fluoro (2′-F) modified nucleotide, or combinations thereof.

In some embodiments, the gRNA comprises one or more, i.e., 1, 2, 3, or 4 modifications at positions 106-109 in the hairpin 2 region. In some embodiments, the gRNA comprises modifications at positions 106-109. In some embodiments, the modification comprises a 2′-O-methyl (2′-O-Me) modified nucleotide.

In some embodiments, the gRNA comprises a 3′ end modification, and a modification in the hairpin region. In some embodiments, the 3′ end modification is within the hairpin region, i.e., in hairpin 2.

In some embodiments, the gRNA comprises a 5′ end modification, and a modification in the hairpin region.

In some embodiments, the gRNA comprises a repeat/anti-repeat modification, and a modification in the hairpin region.

In some embodiments, the gRNA comprises a hairpin modification as shown in any one of the sequences in Table 1 or 2. In some embodiments, such a hairpin modification is combined with a 5′ end modification as shown for the corresponding sequence in Table 1 or 2. In some embodiments, such a hairpin modification is combined with a 3′ end modification as shown for the corresponding sequence in Table 1 or 2. In some embodiments, such a hairpin modification is combined with 5′ and 3′ end modifications as shown for the corresponding sequence in Table 1 or 2.

In some embodiments, the gRNA comprises a 3′ end modification, a modification in the hairpin region, a repeat/anti-repeat modification, and a 5′ end modification.

### Exemplary Guide RNAs

In some embodiments, a gRNA comprising a 5′ end modification and one or more modifications in one or more of: the repeat/anti-repeat region; the hairpin 1 region; and the hairpin 2 region is provided, wherein the one or more modification is at least 99, 98, 97, 96, 95, 94, 93, 92, 91, 90, 85, 80, 75, or 70% identity to the modification pattern shown in the reference sequence identifier in Tables 1-2.

In some embodiments, the gRNAs described herein comprise any of the sequences shown in Tables 1-2. In some embodiments, the gRNAs described herein consist of any of the sequences shown in Tables 1-2. In some embodiments, the gRNAs described herein consist of any of the sequences shown in Tables 1-2 with any 3′ tail sequences removed. Further, gRNAs are encompassed that comprise the modifications of any of the sequences shown in Table 1 or 2, and identified therein by SEQ ID NO. That is, the nucleotides may be the same or different, but the modification pattern shown may be the same or similar to a modification pattern of a guide sequence of Tables 1-2. A modification pattern includes the relative position and identity of modifications of the gRNA.

In some embodiments, the modification pattern contains at least 50%, 55%, 60%, 70%, 75%, preferably at least 80%, 85%, 90%, 95%, 96%, 97%, 98%, or 99% of the modifications of any one of the sequences shown in the sequence column of Tables 1-2, or over one or more regions of the sequence. In some embodiments, the modification pattern is at least 50%, 55%, 60%, 70%, 75%, preferably at least 80%, 85%, 90%, 95%, 96%, 97%, 98%, or 99% identical to the modification pattern of any one of the sequences shown in the sequence column of Tables 1-2. In some embodiments, the modification pattern is at least 50%, 55%, 60%, 70%, 75%, preferably at least 80%, 85%, 90%, or 95% identical to the patterns in Tables 1-2 over one or more (e.g., 1, 2, 3, 4, or 5) regions of the sequence shown in Table 3.

For example, in some embodiments, a gRNA is encompassed wherein the modification pattern is least 50%, 55%, 60%, 70%, 75%, preferably at least 80%, 85%, 90%, or 95% identical to the modification pattern of a sequence over the guide sequence. In some embodiments, a gRNA is encompassed wherein the modification pattern is least 50%, 55%, 60%, 70%, 75%, preferably at least 80%, 85%, 90%, or 95% identical over the repeat/anti-repeat region. In some embodiments, a gRNA is encompassed wherein the modification pattern is least 50%, 55%, 60%, 70%, 75%, preferably at least 80%, 85%, 90%, or 95% identical over the hairpin 1 region. In some embodiments, a gRNA is encompassed wherein the modification pattern is least 50%, 55%, 60%, 70%, 75%, 80%, preferably at least 85%, 90%, 95%, 96%, 97%, 98%, and 99% identical over the hairpin 2 region. In some embodiments, a gRNA is encompassed wherein the modification pattern is least 50%, 55%, 60%, 70%, 80%, or 90%, identical over the 3′ tail. In some embodiments, the modification pattern differs from the modification pattern of a sequence of Tables 1-2, or a region as set forth in Table 3, of such a sequence, at 0, 1, 2, 3, 4, 5, or 6 nucleotides. In some embodiments, the gRNA comprises modifications that differ from the modifications of a sequence of Tables 1-2, at 0, 1, 2, 3, 4, 5, or 6 nucleotides. In some embodiments, the gRNA comprises modifications that differ from modifications of a region set forth in Table 3 of a sequence of Tables 1-2, at 0, 1, 2, 3, 4, 5, or 6 nucleotides.

In some embodiments, a gRNA is provided comprising any one of the sequences described in SEQ ID NOs: 1-19, 21-42, 301-494, 931-946, 951, and 952. In some embodiments, a gRNA is provided consisting of any one of the sequences described in SEQ ID NOs: 1-19, 21-42, 301-494, 931-946, 951, and 952. In some embodiments, a gRNA is provided compromising any one of the sequences described in SEQ ID NOs: 1-19, 21-42, 301-494, 931-946, 951, and 952 including the modifications shown in Tables 1-2. In some embodiments, a gRNA is provided consisting of any one of the sequences described in SEQ ID NOs: 1-19, 21-42, 301-494, 931-946, 951, and 952 including the modifications shown in Tables 1-2. In some embodiments, a gRNA is provided comprising or consisting of any one of the sequences described in SEQ ID NOs: 1-19, 21-42, 301-494, 931-946, 951, and 952 including the modifications shown in Tables 1-2, wherein the 3′ tail, when present, is deleted.

In some embodiments, a gRNA is provided comprising any one of the sequences of SEQ ID NOs: 6 or 9 wherein the gRNA further comprises a guide sequence that is complementary to a target sequence, and directs a Cas9 to its target for cleavage. In some embodiments, a gRNA is provided comprising nucleic acids having at least 99, 98, 97, 96, 95, 94, 93, 92, 91, 90, 85, 80, 75, or 70% identity to the nucleic acids of any one of SEQ ID NOs: 6 or 9, wherein the modification pattern is identical to the modification pattern shown in the reference sequence identifier in Tables 1-2.

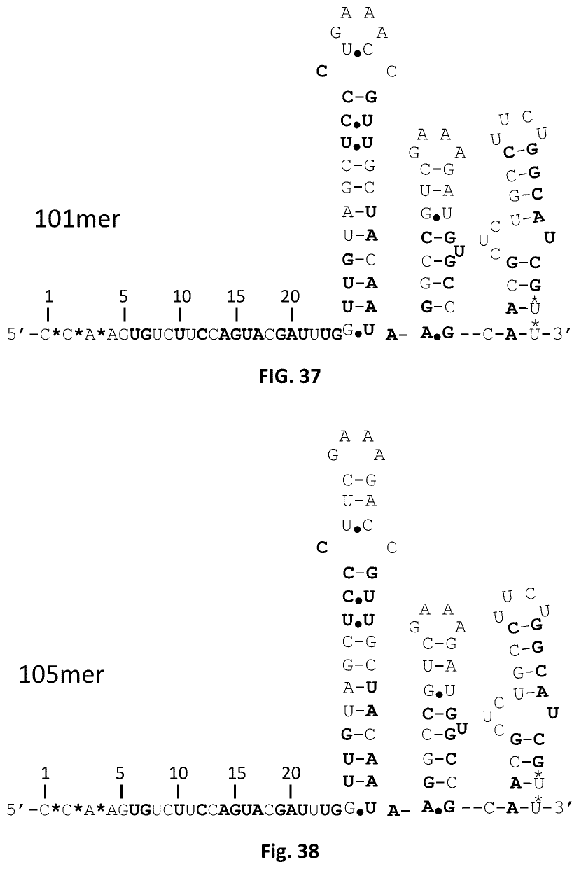

FIGS. 25, 37, and 38 show exemplary sgRNAs in possible secondary structures.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-134 are deleted relative to SEQ ID NO: 500, comprising:
   2′-O-Me modified nucleotides at nucleotides 104, 110, 111, 122-125, 142, and 143,
   PS linkages between nucleotides 141-142 and 142-143, wherein one or both nucleotides 144-145 are optionally deleted relative to SEQ ID NO: 500.

In some embodiments a single guide RNA (sgRNA) is provided, comprising:
a guide region comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide region;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-134 are deleted relative to SEQ ID NO: 500, comprising:
   2′-O-Me modified nucleotides at nucleotides 104, 110, 111, 122-125, 142, and 143;
   PS linkage between nucleotides 141-142 and 142-143,
   wherein one or both nucleotides 144-145 are optionally deleted relative to SEQ ID NO: 500.

In some embodiments a single guide RNA (sgRNA) is provided, comprising:
a guide region comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide region;
a shortened repeat/anti-repeat region, wherein nucleotides 38-47 and 54-63 are deleted relative to SEQ ID NO: 500, comprising:
   2′-O-Me modified nucleotides at nucleotides 25, 29, 30, 31, 32, 37, 48, 49-52, 64, 65, 69, 70, and 73;
a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-134 are deleted relative to SEQ ID NO: 500, comprising:
   2′-O-Me modified nucleotides at nucleotides 104, 110, 111, 122-125, 142, and 143;
   PS linkage between nucleotides 141-142 and 142-143;
   wherein one or both nucleotides 144-145 are optionally deleted relative to SEQ ID NO: 500.

In some embodiments a single guide RNA (sgRNA) comprises:
a guide region comprising:
   2′-O-Me modified nucleotides at the first two nucleotides 1-2;
   PS linkages between nucleotides 1-2; and
   2′-O-Me modified nucleotides at nucleotides 10 and 13 of the guide region;
a shortened repeat/anti-repeat region comprising:
   nucleotides 38-48 and 53-63 are deleted relative to SEQ ID NO: 500;
   2′-O-Me modified nucleotides at nucleotides 25, 29, 30, 31, 32, 37, 49-52, 64, 65, 69, 70, 73;
   a PS linkage between nucleotides 76-77 between the shortened repeat/anti-repeat region and the shortened hairpin 1 region;
a shortened hairpin 1 region comprising:
   nucleotides 86 and 91 are deleted relative to SEQ ID NO: 500;
   2′-O-Me modified nucleotides at nucleotides 80, 81, 83, 84, 85, 87-90, 92-94, 99;
   2′-O-Me modified nucleotide at nucleotide 101 between the shortened hairpin 1 region and the shortened hairpin 2 region;
a shortened hairpin 2 region comprising:
   nucleotides 112-120 and 127-134 are deleted relative to SEQ ID NO: 500;
   2′-O-Me modified nucleotides at nucleotides 102-105, 110, 111, 122-125, 135, 136, 138, 139, 141-143,
   Three PS linkages between nucleotides 140-141, 141-142 and 142-143,
   wherein the sgRNA does not comprise a 3′ tail.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) is provided, comprising:
a guide region comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide region;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 110, 111, 122-125, 142, and 143;
   PS linkage between nucleotides 141-142 and 142-143,
   wherein one or both nucleotides 144-145 are optionally deleted relative to SEQ ID NO: 500.

In some embodiments a single guide RNA (sgRNA) is provided, comprising:
a guide region comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide region;
a shortened repeat/anti-repeat region, wherein nucleotides 38-47 and 54-63 are deleted relative to SEQ ID NO: 500, comprising:
   2′-O-Me modified nucleotides at nucleotides 25, 29, 30, 31, 32, 37, 48, 49-52, 64, 65, 69, 70, and 73;
a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 110, 111, 122-125, 142, and 143;
   PS linkage between nucleotides 141-142 and 142-143;
   wherein one or both nucleotides 144-145 are optionally deleted relative to SEQ ID NO: 500.

In some embodiments a single guide RNA (sgRNA) comprises:
a guide region comprising:
   2′-O-Me modified nucleotides at the first two nucleotides 1-2;
   PS linkages between nucleotides 1-2; and
   2′-O-Me modified nucleotides at nucleotides 10 and 13 of the guide region;
a shortened repeat/anti-repeat region comprising:
   nucleotides 38-48 and 53-63 are deleted relative to SEQ ID NO: 500; 2′-O-Me modified nucleotides at nucleotides 25, 29, 30, 31, 32, 37, 49-52, 64, 65, 69, 70, 73;
   a PS linkage between nucleotides 76-77 between the shortened repeat/anti-repeat region and the shortened hairpin 1 region;
a shortened hairpin 1 region comprising:
   nucleotides 86 and 91 are deleted relative to SEQ ID NO: 500;
   2′-O-Me modified nucleotides at nucleotides 80, 81, 83, 84, 85, 87-90, 92-94, 99;
   2′-O-Me modified nucleotide at nucleotide 101 between the shortened hairpin 1 region and the shortened hairpin 2 region;
a shortened hairpin 2 region comprising:
   nucleotides 112-120 and 127-135 are deleted relative to SEQ ID NO: 500;
   2′-O-Me modified nucleotides at nucleotides 102-105, 110, 111, 122-125, 135, 136, 138, 139, 141-143,
   Three PS linkages between nucleotides 140-141, 141-142 and 142-143,
   wherein the sgRNA does not comprise a 3′ tail.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38, 41-48 and 53-60, and 63 are deleted relative to SEQ ID NO: 500, comprising:
   2′-O-Me modified nucleotides at nucleotides 25, 29, 30, 31, 32, 37, 39-40, 49-52, 61-62, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 106-109, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38-48 and 53-63 are deleted relative to SEQ ID NO: 500, comprising:
   2′-O-Me modified nucleotides at nucleotides 25, 29, 30, 31, 32, 37, 49-52, 64, 65, 69, 70, and 73;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 106-109, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38, 41-48 and 53-60, and 63 are deleted relative to SEQ ID NO: 500, comprising:
   2′-O-Me modified nucleotides at nucleotides 25, 29, 30, 31, 32, 37, 39-40, 49-52, 61-62, 64, 65, 69, 70, and 73;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38, 41-48 and 53-60, and 63 are deleted relative to SEQ ID NO: 500, comprising:
   2′-O-Me modified nucleotides at nucleotides 25, 29, 30, 31, 32, 37, 39-40, 49-52, 61-62, 64, 65, 69, 70, and 73;
   a PS linkage between nucleotides 76-77 between the shortened repeat/anti-repeat region and the shortened hairpin 1 region;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 106-109, 110, 111, 122-125, 142, and 143,
   PS linkages between nucleotides 141-142 and 142-143,
      wherein one or both nucleotides 144-145 are optionally deleted relative to SEQ ID NO: 500.

In some embodiments a single guide RNA (sgRNA) comprises:
a guide sequence comprising:
   2′-O-Me modified nucleotides at the first four nucleotides 1-4;
   PS linkages between nucleotides 1-2, 2-3, and 3-4; and
   2′-O-Me modified nucleotides at nucleotides 5, 8, 9, 11, 13, 18, and 22 of the guide sequence;
a shortened repeat/anti-repeat region, wherein nucleotides 38, 41-48 and 53-60, and 63 are deleted relative to SEQ ID NO: 500, comprising:
   2′-O-Me modified nucleotides at nucleotides 25, 29, 30, 31, 32, 37, 39-40, 49-52, 61-62, 64, 65, 69, 70, and 73;
a shortened hairpin 1 region, wherein nucleotides 86 and 91 are deleted relative to SEQ ID NO: 500, comprising:
   2′-O-Me modified nucleotides at nucleotides 80, 81, 83, 84, 85, 87-90, 92-94, and 99;
2′-O-Me modified nucleotide at nucleotide 101 between the shortened hairpin 1 region and the shortened hairpin 2 region;
a shortened hairpin 2 region, wherein nucleotides 112-120 and 127-135 are deleted relative to SEQ ID NO: 500, comprising:
   2′-O-Me modified nucleotides at nucleotides 104, 106-109, 110, 111, 122-125, 142, and 143,
   PS linkages between nucleotides 141-142 and 142-143,
      wherein one or both nucleotides 144-145 are optionally deleted relative to SEQ ID NO: 500.

### Compositions and Kits

Compositions comprising any of the gRNAs described herein and a carrier, excipient, diluent, or the like are encompassed. In some instances, the excipient or diluent is inert. In some instances, the excipient or diluent is not inert. In certain embodiments, the carrier, excipient, or diluent is non-pyrogenic. In certain embodiments, the carrier, excipient, or diluent is sterile. In some embodiments, a pharmaceutical formulation is provided comprising any of the gRNAs described herein and a pharmaceutically acceptable carrier, excipient, diluent, or the like. In some embodiments, the pharmaceutical formulation further comprises an LNP. In some embodiments, the pharmaceutical formulation further comprises a Cas9 protein or an mRNA encoding a Cas9 protein. In some embodiments, the pharmaceutical formulation comprises any one or more of the gRNAs, an LNP, and a Cas protein or mRNA encoding a Cas protein. In some embodiments, the gRNA is an sgRNA. In some embodiments, the Cas protein is a monomeric Cas protein, e.g., a Cas9 protein. In some embodiments, the Cas protein is an Nine Cas protein. In some embodiments, the Cas protein includes multiple subunits.

Also provided are kits comprising one or more gRNAs, compositions, or pharmaceutical formulations described herein. In some embodiments, a kit further comprises one or more of a solvent, solution, buffer, each separate from the composition or pharmaceutical formulation, instructions, or desiccant.

Compositions Comprising an RNA-Guided DNA Binding Agent or mRNA Encoding RNA-Guided DNA Binding Agent

In some embodiments, compositions or pharmaceutical formulations are provided comprising at least one gRNA, preferably a sgRNA, described herein and an RNA-guided DNA binding agent or a nucleic acid (e.g., an mRNA) encoding an RNA-guided DNA binding agent. In some embodiments, the RNA-guided DNA binding agent is a Cas protein. In some embodiments, the gRNA together with a Cas protein or nucleic acid (e.g., mRNA) encoding Cas protein is called a Cas RNP. In some embodiments, the RNA-guided DNA binding agent is one that functions with the gRNA to direct an RNA-guided DNA binding agent to a target nucleic acid sequence. In some embodiments, the RNA-guided DNA binding agent is a Cas protein from the Type-II CRISPR/Cas system. In some embodiments, the Cas protein is Cas9. In some embodiments, the Cas9 protein is a wild type Cas9. In some embodiments, the Cas9 protein is derived from the Neisseria meningitidis Cas9 (NmeCas9). In some embodiments, compositions are provided comprising at least one gRNA and a nuclease or an mRNA encoding an NmeCas9. In some embodiments, compositions are provided comprising at least one gRNA and a nuclease or an mRNA encoding an NmeCas9. In some embodiments, the Cas induces a double strand break in target DNA. Equivalents of NmeCas9 and its homologs and variants, other Cas proteins disclosed herein are encompassed by the embodiments described herein.

RNA-guided DNA binding agents, including Cas9, encompass modified and variants thereof. Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas, e.g., a nickase Cas9, that induces a nick rather than a double strand break in the target DNA.

In some embodiments, the nuclease, e.g., the RNA-guided DNA binding agent, may be modified to contain only one functional nuclease domain. For example, the RNA-guided DNA binding agent may be modified such that one of the nuclease domains is mutated or fully or partially deleted to reduce its nucleic acid cleavage activity. In some embodiments, a nickase Cas is used having a RuvC domain with reduced activity. In some embodiments, a nickase Cas is used having an inactive RuvC domain. In some embodiments, a nickase Cas is used having an HNH domain with reduced activity. In some embodiments, a nickase Cas is used having an inactive HNH domain.

In some embodiments, a conserved amino acid within an RNA-guided DNA binding agent nuclease domain is substituted to reduce or alter nuclease activity. In some embodiments, a Cas protein may comprise an amino acid substitution in the RuvC or RuvC-like nuclease domain. Exemplary amino acid substitutions in the RuvC or RuvC-like nuclease domain include H588A (based on the N. meningitidis Cas9 protein). In some embodiments, the Cas protein may comprise an amino acid substitution in the HNH or HNH-like nuclease domain. Exemplary amino acid substitutions in the HNH or HNH-like nuclease domain include D16A (based on the NmeCas9 protein).

In some embodiments, the RNP complex described herein comprises a nickase or an mRNA encoding a nickase and a pair of gRNAs (one or both of which may be sgRNAs) that are complementary to the sense and antisense strands of the target sequence, respectively. In this embodiment, the gRNAs (e.g., sgRNAs) direct the nickase to a target sequence and introduce a double stranded break (DSB) by generating a nick on opposite strands of the target sequence (i.e., double nicking). In some embodiments, use of double nicking may improve specificity and reduce off-target effects. In some embodiments, a nickase RNA-guided DNA binding agent is used together with two separate gRNAs (e.g., sgRNAs) that are selected to be in close proximity to produce a double nick in the target DNA.

In some embodiments, chimeric Cas proteins are used, where one domain or region of the protein is replaced by a portion of a different protein. In some embodiments, a Cas nuclease domain may be replaced with a domain from a different nuclease such as Fok1. In some embodiments, a Cas protein may be a modified nuclease.

In some embodiments, the nuclease, e.g., the RNA-guided DNA binding agent, may be modified to induce a point mutation or base change, e.g., a deamination.

In some embodiments, the Cas protein comprises a fusion protein comprising a Cas nuclease (e.g., Cas9), which is a nickase or is catalytically inactive, linked to a heterologous functional domain. In some embodiments, the Cas protein comprises a fusion protein comprising a catalytically inactive Cas nuclease (e.g., Cas9) linked to a heterologous functional domain (see, e.g., WO2014152432). In some embodiments, the catalytically inactive Cas9 is a catalytically inactive N. meningitidis Cas9. In some embodiments, the catalytically inactive Cas comprises mutations that inactivate the Cas. In some embodiments, the heterologous functional domain is a domain that modifies gene expression, histones, or DNA. In some embodiments, the heterologous functional domain is a transcriptional activation domain or a transcriptional repressor domain. In some embodiments, the nuclease is a catalytically inactive Cas nuclease, such as dCas9.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase. A heterologous functional domain such as a deaminase may be part of a fusion protein with a Cas nuclease having nickase activity or a Cas nuclease that is catalytically inactive.

In some embodiments, the target sequence may be adjacent to a PAM. In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Nine Cas9 protein or Nme Cas9 ortholog (Edraki et al., 2019). In some embodiments, the Nine Cas9 PAM may comprise 2, 3, 4, 5, 6, 7, 8, 9, or 10 nucleotides in length. Non-limiting exemplary PAM sequences include NCC, N4GAYW, N4GYTT, N4GTCT, NNNNCC(a), NNNNCAAA (wherein N is defined as any nucleotide, W is defined as either A or T, and R is defined as either A or G; and (a) is a preferred, but not required, A after the second C)). In some embodiments, the PAM sequence may be NCC.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS is preferably fused at the N-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In some embodiments, the NLSs may be fused to the N-terminus of the RNA-guided DNA binding agent sequence. In some embodiments, the NLSs may be fused to only the N-terminus of the RNA-guided DNA binding agent sequence. In some embodiments, the RNA-guided DNA binding agent may have no NLS inserted within the RNA-guided DNA-binding agent sequence. In certain embodiments, may have no NLS C-terminal to the RNA-guided DNA-binding agent sequence.

In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) at the amino terminus. In some embodiments, the RNA-guided DNA-binding agent may be fused with two NLSs, one at the N-terminus and one at the C-terminus. In some embodiments, the RNA-guided DNA-binding agent may be fused with 3 NLSs. In some embodiments, the RNA-guided DNA-binding agent is not fused with an NLS at the C-terminus. In some embodiments, the RNA-guided DNA-binding agent does not include an NLS inserted within the RNA-guided DNA-binding agent sequence. NLS may be fused at the C-terminus of the RNA-guided DNA-binding agent. One or more linkers are optionally included at the fusion site.

In some embodiments, the NLS may be a monopartite sequence, such as, e.g., the SV40 NLS, PKKKRKV (SEQ ID NO: 669) or PKKKRRV (SEQ ID NO: 670). In some embodiments, the NLS may be a bipartite sequence, such as the NLS of nucleoplasmin, KRPAATKKAGQAKKKK (SEQ ID NO: 682). In some embodiments, the NLS sequence may comprise LAAKRSRTT (SEQ ID NO: 671), QAAKRSRTT (SEQ ID NO: 672), PAPAKRERTT (SEQ ID NO: 673), QAAKRPRTT (SEQ ID NO: 674), RAAKRPRTT (SEQ ID NO: 675), AAAKRSWSMAA (SEQ ID NO: 676), AAAKRVWSMAF (SEQ ID NO: 677), AAAKRSWSMAF (SEQ ID NO: 678), AAAKRKYFAA (SEQ ID NO: 679), RAAKRKAFAA (SEQ ID NO: 680), or RAAKRKYFAV (SEQ ID NO: 681). The NLS may be a snurportin-1 importin-β (IBB domain, e.g. an SPN1-impp sequence. See Huber et al., 2002, J. Cell Bio., 156, 467-479. In a specific embodiment, a single PKKKRKV (SEQ ID NO: 669). In some embodiments, the first and second NLS are independently selected from an SV40 NLS, a nucleoplasmin NLS, a bipartite NLS, a c-myc like NLS, and an NLS comprising the sequence KTRAD. In certain embodiments, the first and second NLSs may be the same (e.g., two SV40 NLSs). In certain embodiments, the first and second NLSs may be different.

In some embodiments, the first NLS is a SV40NLS and the second NLS is a nucleoplasmin NLS.

In some embodiments, the SV40 NLS comprises a sequence of SEQ ID NO: 683 or 684. In some embodiments, the nucleoplasmin NLS comprises a sequence of SEQ ID NO: 682. In some embodiments, the bipartite NLS comprises a sequence of SEQ ID NO: 685. In some embodiments, the c-myc like NLS comprises a sequence of SEQ ID NO: 686.

In some embodiments, the RNA-guided DNA binding agent comprises an amino acid sequence with at least 90%, 93%, 95%, 96%, 97%, 98%, 99%, 99.5%, or 100% identity to any one of SEQ ID NOs: 600-603, 605, 607-620, or 707-712 (as shown in Table 4A).

In some embodiments, a polynucleotide encoding the RNA-guided DNA binding agent comprises a nucleotide sequence with at least 90%, 93%, 95%, 96%, 97%, 98%, 99%, 99.5%, or 100% identity to any one of SEQ ID NOs: 621-623, 626-643, 645, 647-668, 701-706, and 713-718 (NmeCas9 mRNA and ORFs as shown in Table 4A).

In some embodiments, the mRNA encoding the RNA-guided DNA binding agent comprises an open reading frame (ORF) comprising a sequence with at least 90%, 93%, 95%, 96%, 97%, 98%, or 99%, or with 100% identity to any one of SEQ ID NOs: 621-623, 626-639, and 713-718 as shown in Table 4A.

### Methods of Use

In some embodiments, any one or more of the gRNAs (e.g., sgRNAs,), compositions, or pharmaceutical formulations described herein is for use in preparing a medicament for treating or preventing a disease or disorder in a subject.

In some embodiments, the invention comprises a method of treating or preventing a disease or disorder in subject comprising administering any one or more of the gRNAs (e.g., sgRNAs), compositions, or pharmaceutical formulations described herein.

In some embodiments, the invention comprises a method or use of modifying a target DNA comprising, administering or delivering any one or more of the gRNAs (e.g., sgRNAs), compositions, or pharmaceutical formulations described herein.

In some embodiments, the invention comprises a method or use for modulation of a target gene comprising, administering or delivering any one or more of the gRNAs (e.g., sgRNAs), compositions, or pharmaceutical formulations described herein. In some embodiments, the modulation is editing of the target gene. In some embodiments, the modulation is a change in expression of the protein encoded by the target gene.

As used herein, a “gene editing” or “genetic modification” is a change at the DNA level, e.g., induced by a gRNA/Cas complex. A gene editing or genetic modification may comprise an insertion, deletion, or substitution (base substitution, e.g., C-to-T, or point mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g., contiguous nucleotides.

In some embodiments, the method or use results in gene editing. In some embodiments, the method or use results in a double-stranded break within the target gene. In some embodiments, the method or use results in formation of indel mutations during non-homologous end joining of the DSB. In some embodiments, the method or use results in an insertion or deletion of nucleotides in a target gene. In some embodiments, the insertion or deletion of nucleotides in a target gene leads to a frameshift mutation or premature stop codon that results in a non-functional protein. In some embodiments, the insertion or deletion of nucleotides in a target gene leads to a knockdown or elimination of target gene expression. In some embodiments, the method or use comprises homology directed repair of a DSB. In some embodiments, the method or use further comprises delivering to the cell a template, wherein at least a part of the template incorporates into a target DNA at or near a double strand break site induced by the nuclease. In some embodiments, the method or use results in a single strand break within the target gene. In some embodiments, the method or use results in a base change, e.g., by deamination, within the target gene. The gene editing typically occurs within or adjacent to the portion of the target gene with which the spacer sequence forms a duplex.

In some embodiments, the method or use results in gene modulation. In some embodiments, the gene modulation is an increase or decrease in gene expression, a change in methylation state of DNA, or modification of a histone subunit. In some embodiments, the method or use results in increased or decreased expression of the protein encoded by the target gene.

The efficacy of gRNAs can be tested in vitro and in vivo. In some embodiments, the invention comprises one or more of the gRNAs, compositions, or pharmaceutical formulations described herein, wherein the gRNA results in gene modulation when provided to a cell together with a Cas nuclease, e.g., Cas9 or mRNA encoding Cas9. In some embodiments, the efficacy of gRNA can be measured in vitro or in vivo.

In some embodiments, the activity of a Cas RNP comprising a gRNA is compared to the activity of a Cas RNP comprising an unmodified sgRNA or a reference sgRNA lacking modifications present in the sgRNA, such as one or more internal linkers, or shortened regions. In some embodiments, the sgRNA do not include an internal linker.

In some embodiments, the efficiency of a gRNA in increasing or decreasing target protein expression is determined by measuring the amount of target protein.

In some embodiments, the efficiency of editing with specific gRNAs is determined by the editing present at the target location in the genome following delivery of a Cas nuclease and the gRNA. In some embodiments, the efficiency of editing with specific gRNAs is measured by next-generation sequencing (NGS). In some embodiments, the editing percentage of the target region of interest is determined. In some embodiments, the total number of sequence reads with sequence alterations, e.g., insertions or deletions (indels), or base changes with no insertion or deletion, of nucleotides into the target region of interest over the total number of sequence reads is measured following delivery of a gRNA and a Cas nuclease.

In some embodiments, the efficiency of editing with specific gRNAs is measured by the presence of sequence alterations, e.g., insertions or deletions, or base substitution, or point mutation of nucleotides introduced by successful gene editing. In some embodiments, activity of a Cas nuclease and gRNAs is tested in biochemical assays. In some embodiments, activity of a Cas nuclease and gRNAs is tested in a cell-free cleavage assay. In some embodiments, activity of a Cas nuclease and gRNAs is tested in Neuro2A cells. In some embodiments, activity of a Cas nuclease and gRNAs is tested in primary cells, e.g., primary hepatocytes.

In some embodiments, the activity of modified gRNAs is measured after in vivo dosing of LNPs comprising modified gRNAs and Cas protein or mRNA encoding Cas protein.

In some embodiments, in vivo efficacy of a gRNA or composition provided herein is determined by editing efficacy measured in DNA extracted from tissue (e.g., liver tissue) after administration of gRNA and a Cas nuclease.

In some embodiments, activation of the subject's immune response is measured by serum concentrations of cytokine(s) following in vivo dosing of sgRNA together with Cas nuclease mRNA or protein (e.g., formulated in an LNP). In some embodiments, the cytokine is interferon-alpha (IFN-alpha), interleukin 6 (IL-6), monocyte chemotactic protein 1 (MCP-1), or tumor necrosis factor alpha (TNF-alpha).

In some embodiments, administration of Cas RNP or Cas nuclease mRNA together with the modified gRNA (e.g., sgRNA) produces lower serum concentration(s) of immune cytokines compared to administration of unmodified sgRNA. In some embodiments, the invention comprises methods comprising administering any one of the gRNAs disclosed herein to a subject, wherein the gRNA elicits a lower concentration of immune cytokines in the subject's serum as compared to a control gRNA that is not similarly modified.

### Delivery of Guide RNA

In some embodiments, the gRNA compositions, compositions, or pharmaceutical formulations disclosed herein, alone or encoded on one or more vectors, are formulated in or administered via a lipid nanoparticle; see e.g., WO2017/173054, the contents of which are hereby incorporated by reference in their entirety.

### Lipids; Formulation; Delivery

Disclosed herein are various embodiments using lipid nucleic acid assembly compositions comprising nucleic acids(s), or composition(s) described herein. In some embodiments, the lipid nucleic acid assembly composition comprises a gRNA described herein, e.g., a gRNA comprising a guide region and a conserved region, the conserved region comprising one or more of: (a) a shortened repeat/anti-repeat region, wherein the shortened repeat/anti-repeat region lacks 2-24 nucleotides, wherein (i) one or more of nucleotides 37-48 and 53-64 is deleted and optionally one or more of nucleotides 37-64 is substituted relative to SEQ ID NO: 500; and (ii) nucleotide 36 is linked to nucleotide 65 by at least 2 nucleotides; or (b) a shortened hairpin 1 region, wherein the shortened hairpin 1 lacks 2-10, optionally 2-8 nucleotides, wherein (i) one or more of nucleotides 82-86 and 91-95 is deleted and optionally one or more of positions 82-96 is substituted relative to SEQ ID NO: 500; and (ii) nucleotide 81 is linked to nucleotide 96 by at least 4 nucleotides; or (c) a shortened hairpin 2 region, wherein the shortened hairpin 2 lacks 2-18, optionally 2-16 nucleotides, wherein (i) one or more of nucleotides 113-121 and 126-134 is deleted and optionally one or more of nucleotides 113-134 is substituted relative to SEQ ID NO: 500; and (ii) nucleotide 112 is linked to nucleotide 135 by at least 4 nucleotides; wherein one or both nucleotides 144-145 are optionally deleted relative to SEQ ID NO: 500; wherein at least 10 nucleotides are modified nucleotides.

As used herein, a “lipid nucleic acid assembly composition” refers to lipid-based delivery compositions, including lipid nanoparticles (LNPs) and lipoplexes. LNP refers to lipid nanoparticles <100 nM. LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e., more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethyl ether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo therapy. In some embodiments, the aqueous solution comprises a gRNA described herein. In some embodiments, the aqueous solution further comprises an mRNA encoding an RNA-guided DNA binding agent, such as Cas9.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides to subjects may be utilized with the guide RNAs and the nucleic acid encoding an RNA-guided nickase and the nucleic acid encoding a cytidine deaminase described herein.

In some embodiments, the aqueous solution comprises a gRNA described herein and optionally further comprises an mRNA encoding an RNA-guided DNA binding agent, such as Cas9. A pharmaceutical formulation comprising the lipid nucleic acid assembly composition may optionally comprise a pharmaceutically acceptable buffer.

In some embodiments, the lipid nucleic acid assembly compositions include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

### Amine Lipids

In some embodiments, lipid nucleic acid assembly compositions comprise an “amine lipid”, which is, for example an ionizable lipid such as Lipid A or its equivalents, including acetal analogs of Lipid A.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

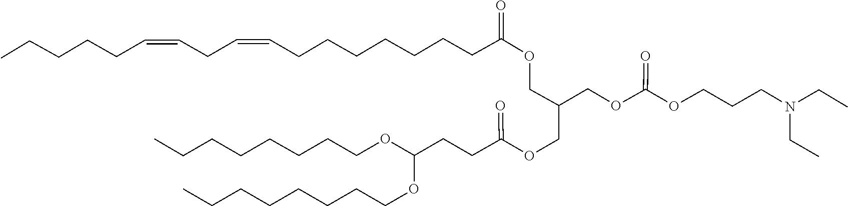 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is an equivalent to Lipid A.

In some embodiments, an amine lipid is an analog of Lipid A. In some embodiments, a Lipid A analog is an acetal analog of Lipid A. In particular lipid nucleic acid assembly compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g., an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO/2020/219876, WO/2020/118041, WO/2020/072605, WO/2019/067992, WO/2017/173054, WO2015/095340, and WO2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week-old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of lipid nucleic acid assembly compositions of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

### Additional Lipids

“Neutral lipids” suitable for use in a lipid nucleic acid assembly composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid nucleic acid assembly composition of the disclosure include, but are not limited to, stealth lipids having a hydrophilic head group linked to a lipid moiety. Stealth lipids suitable for use in a lipid nucleic acid assembly composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (I), wherein n is 45, meaning that the number averaged degree of polymerization comprises about 45 subunits

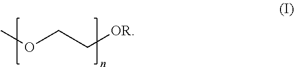

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (e.g., 1,2-dimyristoyl-rac-glycero-3-methylpolyoxyethylene glycol 2000 (PEG2k-DMG) or PEG-DMG (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMG) (cat. #880150P from Avanti Polar Lipids, Alabaster, Alabama, USA), 1,2-distearoyl-sn-glycero-3-phosphoethanolamine-N-[methoxy(polyethylene glycol)-2000](PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be 1,2-dimyristoyl-rac-glycero-3-methylpolyoxyethylene glycol 2000 (PEG2k-DMG). In one embodiment, the PEG lipid may be PEG2k-DMG. In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In preferred embodiments, the PEG lipid includes a glycerol group. In preferred embodiments, the PEG lipid includes a dimyristoylglycerol (DMG) group. In preferred embodiments, the PEG lipid comprises PEG-2k. In preferred embodiments, the PEG lipid is a PEG-DMG. In preferred embodiments, the PEG lipid is a PEG-2k-DMG. In preferred embodiments, the PEG lipid is 1,2-dimyristoyl-rac-glycero-3-methoxypolyethylene glycol2000. In preferred embodiments, the PEG-2k-DMG is 1,2-dimyristoyl-rac-glycero-3-methoxypolyethylene glycol-2000.

LNP Delivery of gRNA

Lipid nanoparticles (LNPs) are a well-known means for delivery of nucleotide and protein cargo, and may be used for delivery of the gRNAs (e.g., sgRNAs), compositions, or pharmaceutical formulations disclosed herein. In some embodiments, the LNPs deliver nucleic acid, protein, or nucleic acid together with protein. As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical and, in more particular embodiments, can comprise an aqueous core, e.g., comprising a substantial portion of RNA molecules), a dispersed phase in an emulsion, micelles, or an internal phase in a suspension (see, e.g., WO2017173054, the contents of which are hereby incorporated by reference in their entirety). Any LNP known to those of skill in the art to be capable of delivering nucleotides to subjects may be utilized.

In some embodiments, the invention comprises a method for delivering any one of the gRNAs disclosed herein to a subject, wherein the gRNA is associated with an LNP. In some embodiments, the gRNA/LNP is also associated with a Cas nuclease or a polynucleotide (e.g., mRNA or DNA) encoding a Cas nuclease.

In some embodiments, the invention comprises a composition comprising any one of the gRNAs disclosed and an LNP. In some embodiments, the composition further comprises a Cas9 or a polynucleotide (e.g., mRNA or DNA) encoding Cas9.

In some embodiments, provided herein is a method for delivering any of the guide RNAs described herein to a cell or a population of cells or a subject, including to a cell or population of cells in a subject in vivo, wherein any one or more of the components is associated with an LNP. In some embodiments, the method further comprises an RNA-guided DNA-binding agent (e.g., Cas9 or a polynucleotide (e.g., mRNA or DNA) encoding Cas9).

In some embodiments, provided herein is a composition comprising any of the guide RNAs described herein or donor construct disclosed herein, alone or in combination, with an LNP. In some embodiments, the composition further comprises an RNA-guided DNA-binding agent (e.g., Cas9 or a polynucleotide (e.g., mRNA or DNA) encoding Cas9).

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). In some embodiments, the LNPs comprise molar ratios of a cationic lipid amine to RNA phosphate (N:P) of about 4.5. In some embodiments, the LNPs comprise is nonyl 8-((7,7-bis(octyloxy)heptyl)(2-hydroxyethyl)amino)octanoate. In some embodiments, the LNPs comprise molar ratios of a cationic lipid amine to RNA phosphate (N:P) of about 4.5-6.5. In some embodiments, the LNPs comprise molar ratios of a cationic lipid amine to RNA phosphate (N:P) of about 4.5. In some embodiments, the LNPs comprise molar ratios of a cationic lipid amine to RNA phosphate (N:P) of about 6.0.

In some embodiments, LNPs associated with the gRNAs disclosed herein are for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or a polynucleotide (e.g., mRNA or DNA) encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or a polynucleotide (e.g., mRNA or DNA) encoding Cas9. (See, e.g., PCT/US2021/029446, incorporated herein by reference)

In some embodiments, the vector comprises one or more nucleotide sequence(s) encoding an mRNA encoding an RNA-guided DNA nuclease, which can be a Cas nuclease, such as NmeCas9. In some embodiments, the vector comprises an mRNA encoding an RNA-guided DNA nuclease, which can be a Cas protein, such as Cas9. In one embodiment, the Cas9 is NmeCas9.

In some embodiments, the components can be introduced as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

In some embodiments, LNPs associated with the gRNAs disclosed herein are for use in preparing a medicament for treating a disease or disorder.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified.

TABLE 4A
Table of Sequences

| SEQ ID | | |
|---|---|---|
| No. | Description | Sequence |
| 600 | Amino acid | MTGAAFKPNPINYILGLDIGIASVGWAMVEIDEEENPIRLIDLGVRVFERAEVPK |
| | sequence for | TGDSLAMARRLARSVRRLTRRRAHRLLRARRLLKREGVLQAADFDENGLIKSLPN |
| | Nme2Cas9 | TPWQLRAAALDRKLTPLEWSAVLLHLIKHRGYLSQRKNEGETADKELGALLKGVA |
| | encoded | NNAHALQTGDFRTPAELALNKFEKESGHIRNQRGDYSHTFSRKDLQAELILLFEK |
| | by mRNA A | QKEFGNPHVSGGLKEGIETLLMTQRPALSGDAVQKMLGHCTFEPAEPKAAKNTYT |
| | | AERFIWLTKLNNLRILEQGSERPLTDTERATLMDEPYRKSKLTYAQARKLLGLED |
| | | TAFFKGLRYGKDNAEASTLMEMKAYHAISRALEKEGLKDKKSPLNLSSELQDEIG |
| | | TAFSLFKTDEDITGRLKDRVQPEILEALLKHISFDKFVQISLKALRRIVPLMEQG |
| | | KRYDEACAEIYGDHYGKKNTEEKIYLPPIPADEIRNPVVLRALSQARKVINGVVR |
| | | RYGSPARIHIETAREVGKSFKDRKEIEKRQEENRKDREKAAAKFREYFPNFVGEP |
| | | KSKDILKLRLYEQQHGKCLYSGKEINLVRLNEKGYVEIDHALPESRTWDDSFNNK |
| | | VLVLGSENQNKGNQTPYEYENGKDNSREWQEFKARVETSREPRSKKQRILLQKED |
| | | EDGFKECNLNDTRYVNRFLCQFVADHILLTGKGKRRVFASNGQITNLLRGFWGLR |
| | | KVRAENDRHHALDAVVVACSTVAMQQKITRFVRYKEMNAFDGKTIDKETGKVLHQ |
| | | KTHFPQPWEFFAQEVMIRVFGKPDGKPEFEEADTPEKLRTLLAEKLSSRPEAVHE |
| | | YVTPLFVSRAPNRKMSGAHKDTLRSAKRFVKHNEKISVKRVWLTEIKLADLENMV |
| | | NYKNGREIELYEALKARLEAYGGNAKQAFDPKDNPFYKKGGQLVKAVRVEKTQES |
| | | GVLLNKKNAYTIADNGDMVRVDVFCKVDKKGKNQYFIVPIYAWQVAENILPDIDC |
| | | KGYRIDDSYTFCFSLHKYDLIAFQKDEKSKVEFAYYINCDSSNGRFYLAWHDKGS |
| | | KEQQFRISTQNLVLIQKYQVNELGKEIRPCRLKKRPPVRSGKRTADGSEFESPKK |
| | | KRKVE |
| 601 | Amino acid | MTGAAFKPNPINYILGLDIGIASVGWAMVEIDEEENPIRLIDLGVRVFERAEVPK |
| | sequence for | TGDSLAMARRLARSVRRLTRRRAHRLLRARRLLKREGVLQAADFDENGLIKSLPN |
| | Nme2Cas9 | TPWQLRAAALDRKLTPLEWSAVLLHLIKHRGYLSQRKNEGETADKELGALLKGVA |
| | encoded | NNAHALQTGDFRTPAELALNKFEKESGHIRNQRGDYSHTFSRKDLQAELILLFEK |
| | by mRNA B | QKEFGNPHVSGGLKEGIETLLMTQRPALSGDAVQKMLGHCTFEPAEPKAAKNTYT |
| | | AERFIWLTKLNNLRILEQGSERPLTDTERATLMDEPYRKSKLTYAQARKLLGLED |
| | | TAFFKGLRYGKDNAEASTLMEMKAYHAISRALEKEGLKDKKSPLNLSSELQDEIG |
| | | TAFSLFKTDEDITGRLKDRVQPEILEALLKHISFDKFVQISLKALRRIVPLMEQG |
| | | KRYDEACAEIYGDHYGKKNTEEKIYLPPIPADEIRNPVVLRALSQARKVINGVVR |
| | | RYGSPARIHIETAREVGKSFKDRKEIEKRQEENRKDREKAAAKFREYFPNFVGEP |
| | | KSKDILKLRLYEQQHGKCLYSGKEINLVRLNEKGYVEIDHALPESRTWDDSFNNK |
| | | VLVLGSENQNKGNQTPYEYENGKDNSREWQEFKARVETSRFPRSKKQRILLQKFD |
| | | EDGEKECNLNDTRYVNRFLCQFVADHILLTGKGKRRVFASNGQITNLLRGFWGLR |
| | | KVRAENDRHHALDAVVVACSTVAMQQKITRFVRYKEMNAFDGKTIDKETGKVLHQ |
| | | KTHFPQPWEFFAQEVMIRVFGKPDGKPEFEEADTPEKLRTLLAEKLSSRPEAVHE |
| | | YVTPLFVSRAPNRKMSGAHKDTLRSAKRFVKHNEKISVKRVWLTEIKLADLENMV |
| | | NYKNGREIELYEALKARLEAYGGNAKQAFDPKDNPFYKKGGQLVKAVRVEKTQES |
| | | GVLLNKKNAYTIADNGDMVRVDVFCKVDKKGKNQYFIVPIYAWQVAENILPDIDC |
| | | KGYRIDDSYTFCFSLHKYDLIAFQKDEKSKVEFAYYINCDSSNGRFYLAWHDKGS |
| | | KEQQFRISTQNLVLIQKYQVNELGKEIRPCRLKKRPPVRSGKRTADGSEFESPKK |
| | | KRKVE |
| 602 | Amino acid | MTGAAFKPNPINYILGLDIGIASVGWAMVEIDEEENPIRLIDLGVRVFERAEVPK |
| | sequence for | TGDSLAMARRLARSVRRLTRRRAHRLLRARRLLKREGVLQAADFDENGLIKSLPN |
| | Nme2Cas9 | TPWQLRAAALDRKLTPLEWSAVLLHLIKHRGYLSQRKNEGETADKELGALLKGVA |
| | encoded | NNAHALQTGDFRTPAELALNKFEKESGHIRNQRGDYSHTFSRKDLQAELILLFEK |
| | by mRNA C | QKEFGNPHVSGGLKEGIETLLMTQRPALSGDAVQKMLGHCTFEPAEPKAAKNTYT |
| | | AERFIWLTKLNNLRILEQGSERPLTDTERATLMDEPYRKSKLTYAQARKLLGLED |
| | | TAFFKGLRYGKDNAEASTLMEMKAYHAISRALEKEGLKDKKSPLNLSSELQDEIG |
| | | TAFSLEKTDEDITGRLKDRVQPEILEALLKHISFDKFVQISLKALRRIVPLMEQG |
| | | KRYDEACAEIYGDHYGKKNTEEKIYLPPIPADEIRNPVVLRALSQARKVINGVVR |
| | | RYGSPARIHIETAREVGKSFKDRKEIEKRQEENRKDREKAAAKFREYFPNFVGEP |
| | | KSKDILKLRLYEQQHGKCLYSGKEINLVRLNEKGYVEIDHALPESRTWDDSFNNK |
| | | VLVLGSENQNKGNQTPYEYENGKDNSREWQEFKARVETSRFPRSKKQRILLQKFD |
| | | EDGEKECNLNDTRYVNRFLCQFVADHILLTGKGKRRVFASNGQITNLLRGFWGLR |
| | | KVRAENDRHHALDAVVVACSTVAMQQKITRFVRYKEMNAFDGKTIDKETG |
| | | KVLHQKTHFPQPWEFFAQEVMIRVFGKPDGKPEFEEADTPEKLRTLLAEKLSSRP |
| | | EAVHEYVTPLFVSRAPNRKMSGAHKDTLRSAKRFVKHNEKISVKRVWLTEIKLAD |
| | | LENMVNYKNGREIELYEALKARLEAYGGNAKQAFDPKDNPFYKKGGQLVKAVRVE |
| | | KTQESGVLLNKKNAYTIADNGDMVRVDVFCKVDKKGKNQYFIVPIYAWQVAENIL |
| | | PDIDCKGYRIDDSYTFCFSLHKYDLIAFQKDEKSKVEFAYYINCDSSNGRFYLAW |
| | | HDKGSKEQQFRISTQNLVLIQKYQVNELGKEIRPCRLKKRPPVRSGKRTADGSEF |
| | | ESPKKKRKVE |
| 603 | Amino acid | MTGAAFKPNPINYILGLDIGIASVGWAMVEIDEEENPIRLIDLGVRVFERAEVPK |
| | sequence for | TGDSLAMARRLARSVRRLTRRRAHRLLRARRLLKREGVLQAADEDENGLIKSLPN |
| | Nme2Cas9 | TPWQLRAAALDRKLTPLEWSAVLLHLIKHRGYLSQRKNEGETADKELGALLKGVA |
| | encoded | NNAHALQTGDFRTPAELALNKFEKESGHIRNQRGDYSHTFSRKDLQAELILLFEK |
| | by mRNA D | QKEFGNPHVSGGLKEGIETLLMTQRPALSGDAVQKMLGHCTFEPAEPKAAKNTYT |
| | | AERFIWLTKLNNLRILEQGSERPLTDTERATLMDEPYRKSKLTYAQARKLLGLED |
| | | TAFFKGLRYGKDNAEASTLMEMKAYHAISRALEKEGLKDKKSPLNLSSELQDEIG |
| | | TAFSLFKTDEDITGRLKDRVQPEILEALLKHISFDKFVQISLKALRRIVPLMEQG |
| | | KRYDEACAEIYGDHYGKKNTEEKIYLPPIPADEIRNPVVLRALSQARKVINGVVR |
| | | RYGSPARIHIETAREVGKSFKDRKEIEKRQEENRKDREKAAAKFREYFPNFVGEP |
| | | KSKDILKLRLYEQQHGKCLYSGKEINLVRLNEKGYVEIDHALPESRTWDDSENNK |
| | | VLVLGSENQNKGNQTPYEYENGKDNSREWQEFKARVETSREPRSKKQRILLQKFD |
| | | EDGEKECNLNDTRYVNRELCQFVADHILLTGKGKRRVFASNGQITNLLRGFWGLR |
| | | KVRAENDRHHALDAVVVACSTVAMQQKITRFVRYKEMNAFDGKTIDKETGKVLHQ |
| | | KTHFPQPWEFFAQEVMIRVFGKPDGKPEFEEADTPEKLRTLLAEKLSSRPEAVHE |
| | | YVTPLFVSRAPNRKMSGAHKDTLRSAKRFVKHNEKISVKRVWLTEIKLADLENMV |
| | | NYKNGREIELYEALKARLEAYGGNAKQAFDPKDNPFYKKGGQLVKAVRVEKTQES |
| | | GVLLNKKNAYTIADNGDMVRVDVFCKVDKKGKNQYFIVPIYAWQVAENILPDIDC |
| | | KGYRIDDSYTFCFSLHKYDLIAFQKDEKSKVEFAYYINCDSSNGRFYLAWHDKGS |
| | | KEQQFRISTQNLVLIQKYQVNELGKEIRPCRLKKRPPVRSGKRTADGSEFESPKK |
| | | KRKVE |
| 604 | Amino acid | MEASPASGPRHLMDPHIFTSNENNGIGRHKTYLCYEVERLDNGTSVKMDQHRGFL |
| | sequence for | HNQAKNLLCGFYGRHAELRELDLVPSLQLDPAQIYRVTWFISWSPCFSWGCAGEV |
| | SpyCas9 base | RAFLQENTHVRLRIFAARIYDYDPLYKEALQMLRDAGAQVSIMTYDEFKHCWDTF |
| | editor encoded | VDHQGCPFQPWDGLDEHSQALSGRLRAILQNQGNSGSETPGTSESATPESDKKYS |
| | by mRNA E | IGLAIGTNSVGWAVITDEYKVPSKKFKVLGNTDRHSIKKNLIGALLFDSGETAEA |
| | | TRLKRTARRRYTRRKNRICYLQEIFSNEMAKVDDSFFHRLEESFLVEEDKKHERH |
| | | PIFGNIVDEVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDDLDNLLAQ |
| | | IGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLV |
| | | KLNREDLLRKQRTFDNGSIPHQIHLGELHAILRRQEDFYPELKDNREKIEKILTF |
| | | RIPYYVGPLARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKAIVDLLFKTN |
| | | RKVTVKQLKEDYFKKIECFDSVEISGVEDRENASLGTYHDLLKIIKDKDELDNEE |
| | | NEDILEDIVLTLTLFEDREMIEERLKTYAHLFDDKVMKQLKRRRYTGWGRLSRKL |
| | | INGIRDKQSGKTILDELKSDGFANRNFMQLIHDDSLTFKEDIQKAQVSGQGDSLH |
| | | EHIANLAGSPAIKKGILQTVKVVDELVKVMGRHKPENIVIEMARENQTTQKGQKN |
| | | SRERMKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNYWRQL |
| | | LNAKLITQRKFDNLTKAERGGLSELDKAGFIKRQLVETRQITKHVAQILDSRMNT |
| | | KYDENDKLIREVKVITLKSKLVSDERKDFQFYKVREINNYHHAHDAYLNAVVGTA |
| | | LIKKYPKLESEFVYGDYKVYDVRKMIAKSEQEIGKATAKYFFYSNIMNFFKTEIT |
| | | LANGEIRKRPLIETNGETGEIVWDKGRDFATVRKVLSMPQVNIVKKTEVQTGGES |
| | | KESILPKRNSDKLIARKKDWDPKKYGGEDSPTVAYSVLVVAKVEKGKSKKLKSVK |
| | | ELLGITIMERSSFEKNPIDELEAKGYKEVKKDLIIKLPKYSLFELENGRKRMLAS |
| | | AGELQKGNELALPSKYVNFLYLASHYEKLKGSPEDNEQKQLFVEQHKHYLDEIIE |
| | | QISEFSKRVILADANLDKVLSAYNKHRDKPIREQAENIIHLETLTNLGAPAAFKY |
| | | FDTTIDRKRYTSTKEVLDATLIHQSITGLYETRIDLSQLGGDGGGSPKKKRKV |
| 605 | Amino acid | MAAFKPNSINYILGLDIGIASVGWAMVEIDEEENPIRLIDLGVRVFERAEVPKTG |
| | sequence for | DSLAMARRLARSVRRLTRRRAHRLLRTRRLLKREGVLQAANFDENGLIKSLPNTP |
| | Nme1Cas9 | WQLRAAALDRKLTPLEWSAVLLHLIKHRGYLSQRKNEGETADKELGALLKGVAGN |
| | encoded | AHALQTGDFRTPAELALNKFEKESGHIRNQRSDYSHTESRKDLQAELILLFEKQK |
| | by mRNA F | EFGNPHVSGGLKEGIETLLMTQRPALSGDAVQKMLGHCTFEPAEPKAAKNTYTAE |
| | | RFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSPELQDEIGTA |
| | | FSLEKTDEDITGRLKDRIQPEILEALLKHISFDKFVQISLKALRRIVPLMEQGKR |
| | | YDEACAEIYGDHYGKKNTEEKIYLPPIPADEIRNPVVLRALSQARKVINGVVRRY |
| | | GSPARIHIETAREVGKSFKDRKEIEKRQEENRKDREKAAAKFREYFPNFVGEPKS |
| | | KDILKLRLYEQQHGKCLYSGKEINLGRLNEKGYVEIDHALPESRTWDDSFNNKVL |
| | | VLGSENQNKGNQTPYEYENGKDNSREWQEFKARVETSRFPRSKKQRILLQKFDED |
| | | GEKERNLNDTRYVNRFLCQFVADRMRLTGKGKKRVFASNGQITNLLRGFWGLRKV |
| | | RAENDRHHALDAVVVACSTVAMQQKITRFVRYKEMNAFDGKTIDKETGEVLHQKT |
| | | HFPQPWEFFAQEVMIRVFGKPDGKPEFEEADTLEKLRTLLAEKLSSRPEAVHEYV |
| | | TPLFVSRAPNRKMSGQGHMETVKSAKRLDEGVSVLRVPLTQLKLKDLEKMVNRER |
| | | EPKLYEALKARLEAHKDDPAKAFAEPFYKYDKAGNRTQQVKAVRVEQVQKTGVWV |
| | | RNHNGIADNATMVRVDVFEKGDKYYLVPIYSWQVAKGILPDRAVVQGKDEEDWQL |
| | | IDDSENFKFSLHPNDLVEVITKKARMEGYEASCHRGTGNINIRIHDLDHKIGKNG |
| | | ILEGIGVKTALSFQKYQIDELGKEIRPCRLKKRPPVRSGKRTADGSEFESPKKKR |
| | | KVE |
| 606 | Amino acid | MTNLSDIIEKETGKQLVIQESILMLPEEVEEVIGNKPESDILVHTAYDESTDENV |
| | sequence | MLLTSDAPEYKPWALVIQDSNGENKIKMLSGGSKRTADGSEFESPKKKRKVE |
| | for UGI | |
| | encoded by | |
| | mRNA G | |
| 607 | Amino acid | MAAFKPNPINYILGLDIGIASVGWAMVEIDEEENPIRLIDLGVRVFERAEVPKTG |
| | sequence for | DSLAMARRLARSVRRLTRRRAHRLLRARRLLKREGVLQAADEDENGLIKSLPNTP |
| | Nme2Cas9 | WQLRAAALDRKLTPLEWSAVLLHLIKHRGYLSQRKNEGETADKELGALLKGVANN |
| | encoded | AHALQTGDFRTPAELALNKFEKESGHIRNQRGDYSHTFSRKDLQAELILLFEKQK |
| | by mRNA H | EFGNPHVSGGLKEGIETLLMTQRPALSGDAVQKMLGHCTFEPAEPKAAKNTYTAE |
| | | RFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSSELQDEIGTA |
| | | FSLEKTDEDITGRLKDRVQPEILEALLKHISFDKFVQISLKALRRIVPLMEQGKR |
| | | YDEACAEIYGDHYGKKNTEEKIYLPPIPADEIRNPVVLRALSQARKVINGVVRRY |
| | | GSPARIHIETAREVGKSFKDRKEIEKRQEENRKDREKAAAKFREYFPNFVGEPKS |
| | | KDILKLRLYEQQHGKCLYSGKEINLVRLNEKGYVEIDHALPESRTWDDSFNNKVL |
| | | VLGSENQNKGNQTPYEYENGKDNSREWQEFKARVETSRFPRSKKQRILLQKEDED |
| | | GEKECNLNDTRYVNRFLCQFVADHILLTGKGKRRVFASNGQITNLLRGFWGLRKV |
| | | RAENDRHHALDAVVVACSTVAMQQKITRFVRYKEMNAFDGKTIDKETGKVLHQKT |
| | | HFPQPWEFFAQEVMIRVFGKPDGKPEFEEADTPEKLRTLLAEKLSSRPEAVHEYV |
| | | TPLFVSRAPNRKMSGAHKDTLRSAKRFVKHNEKISVKRVWLTEIKLADLENMVNY |
| | | KNGREIELYEALKARLEAYGGNAKQAFDPKDNPFYKKGGQLVKAVRVEKTQESGV |
| | | LLNKKNAYTIADNGDMVRVDVFCKVDKSGGGSPKKKRKVSGGSGKNQYFIVPIYA |
| | | WQVAENILPDIDCKGYRIDDSYTFCFSLHKYDLIAFQKDEKSKVEFAYYINCDSS |
| | | NGRFYLAWHDKGSKEQQFRISTQNLVLIQKYQVNELGKEIRPCRLKKRPPVR |
| 608 | Amino acid | MVPKKKRKVAAFKPNPINYILGLDIGIASVGWAMVEIDEEENPIRLIDLGVRVFE |
| | sequence for | RAEVPKTGDSLAMARRLARSVRRLTRRRAHRLLRARRLLKREGVLQAADEDENGL |
| | Nme2Cas9 | IKSLPNTPWQLRAAALDRKLTPLEWSAVLLHLIKHRGYLSQRKNEGETADKELGA |
| | encoded | LLKGVANNAHALQTGDFRTPAELALNKFEKESGHIRNQRGDYSHTFSRKDLQAEL |
| | by mRNA I | ILLFEKQKEFGNPHVSGGLKEGIETLLMTQRPALSGDAVQKMLGHCTFEPAEPKA |
| | | AKNTYTAERFIWLTKLNNLRILEQGSERPLTDTERATLMDEPYRKSKLTYAQARK |
| | | LLGLEDTAFFKGLRYGKDNAEASTLMEMKAYHAISRALEKEGLKDKKSPLNLSSE |
| | | LQDEIGTAFSLFKTDEDITGRLKDRVQPEILEALLKHISEDKFVQISLKALRRIV |
| | | PLMEQGKRYDEACAEIYGDHYGKKNTEEKIYLPPIPADEIRNPVVLRALSQARKV |
| | | INGVVRRYGSPARIHIETAREVGKSFKDRKEIEKRQEENRKDREKAAAKFREYFP |
| | | NFVGEPKSKDILKLRLYEQQHGKCLYSGKEINLVRLNEKGYVEIDHALPFSRTWD |
| | | DSFNNKVLVLGSENQNKGNQTPYEYENGKDNSREWQEFKARVETSRFPRSKKQRI |
| | | LLQKEDEDGEKECNLNDTRYVNRFLCQFVADHILLTGKGKRRVFASNGQITNLLR |
| | | GFWGLRKVRAENDRHHALDAVVVACSTVAMQQKITRFVRYKEMNAFDGKTIDKET |
| | | GKVLHQKTHFPQPWEFFAQEVMIRVFGKPDGKPEFEEADTPEKLRTLLAEKLSSR |
| | | PEAVHEYVTPLFVSRAPNRKMSGAHKDTLRSAKRFVKHNEKISVKRVWLTEIKLA |
| | | DLENMVNYKNGREIELYEALKARLEAYGGNAKQAFDPKDNPFYKKGGQLVKAVRV |
| | | EKTQESGVLLNKKNAYTIADNGDMVRVDVFCKVDKKGKNQYFIVPIYAWQVAENI |
| | | LPDIDCKGYRIDDSYTFCFSLHKYDLIAFQKDEKSKVEFAYYINCDSSNGRFYLA |
| | | WHDKGSKEQQFRISTQNLVLIQKYQVNELGKEIRPCRLKKRPPVRYPYDVPDYAA |
| | | APAAKKKKLD |
| 609 | Amino acid | MAAFKPNPINYILGLDIGIASVGWAMVEIDEEENPIRLIDLGVRVFERAEVPKTG |
| | sequence for | DSLAMARRLARSVRRLTRRRAHRLLRARRLLKREGVLQAADFDENGLIKSLPNTP |
| | Nme2Cas9 | WQLRAAALDRKLTPLEWSAVLLHLIKHRGYLSQRKNEGETADKELGALLKGVANN |
| | encoded | AHALQTGDFRTPAELALNKFEKESGHIRNQRGDYSHTFSRKDLQAELILLFEKQK |
| | by mRNA J | EFGNPHVSGGLKEGIETLLMTQRPALSGDAVQKMLGHCTFEPAEPKAAKNTYTAE |
| | | RFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSSELQDEIGTA |
| | | FSLEKTDEDITGRLKDRVQPEILEALLKHISFDKFVQISLKALRRIVPLMEQGKR |
| | | YDEACAEIYGDHYGKKNTEEKIYLPPIPADEIRNPVVLRALSQARKVINGVVRRY |
| | | GSPARIHIETAREVGKSFKDRKEIEKRQEENRKDREKAAAKFREYEPNFVGEPKS |
| | | KDILKLRLYEQQHGKCLYSGKEINLVRLNEKGYVEIDHALPFSRTWDDSFNNKVL |
| | | VLGSENQNKGNQTPYEYENGKDNSREWQEFKARVETSREPRSKKQRILLQKFDED |
| | | GFKECNLNDTRYVNRFLCQFVADHILLTGKGKRRVFASNGQITNLLRGFWGLRKV |
| | | RAENDRHHALDAVVVACSTVAMQQKITRFVRYKEMNAFDGKTIDKETGKVLHQKT |
| | | HFPQPWEFFAQEVMIRVFGKPDGKPEFEEADTPEKLRTLLAEKLSSRPEAVHEYV |
| | | TPLFVSRAPNRKMSGAHKDTLRSAKRFVKHNEKISVKRVWLTEIKLADLENMVNY |
| | | KNGREIELYEALKARLEAYGGNAKQAFDPKDNPFYKKGGQLVKAVRVEKTQESGV |
| | | LLNKKNAYTIADNGDMVRVDVFCKVDKKGKNQYFIVPIYAWQVAENILPDIDCKG |
| | | YRIDDSYTFCFSLHKYDLIAFQKDEKSKVEFAYYINCDSSNGRFYLAWHDKGSKE |
| | | QQFRISTQNLVLIQKYQVNELGKEIRPCRLKKRPPVR |
| 610 | Amino acid | MAAFKPNPINYILGLDIGIASVGWAMVEIDEEENPIRLIDLGVRVFERAEVPKTG |
| | sequence for | DSLAMARRLARSVRRLTRRRAHRLLRARRLLKREGVLQAADFDENGLIKSLPNTP |
| | Nme2Cas9 | WQLRAAALDRKLTPLEWSAVLLHLIKHRGYLSQRKNEGETADKELGALLKGVANN |
| | encoded | AHALQTGDFRTPAELALNKFEKESGHIRNQRGDYSHTFSRKDLQAELILLFEKQK |
| | by mRNA K | EFGNPHVSGGLKEGIETLLMTQRPALSGDAVQKMLGHCTFEPAEPKAAKNTYTAE |
| | | RFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSSELQDEIGTA |
| | | FSLEKTDEDITGRLKDRVQPEILEALLKHISFDKFVQISLKALRRIVPLMEQGKR |
| | | YDEACAEIYGDHYGKKNTEEKIYLPPIPADEIRNPVVLRALSQARKVINGVVRRY |
| | | GSPARIHIETAREVGKSFKDRKEIEKRQEENRKDREKAAAKFREYFPNFVGEPKS |
| | | KDILKLRLYEQQHGKCLYSGKEINLVRLNEKGYVEIDHALPFSRTWDDSFNNKVL |
| | | VLGSENQNKGNQTPYEYFNGKDNSREWQEFKARVETSRFPRSKKQRILLQKFDED |
| | | GEKECNLNDTRYVNRFLCQFVADHILLTGKGKRRVFASNGQITNLLRGFWGLRKV |
| | | RAENDRHHALDAVVVACSTVAMQQKITRFVRYKEMNAFDGKTIDKETGKVLHQKT |
| | | HFPQPWEFFAQEVMIRVFGKPDGKPEFEEADTPEKLRTLLAEKLSSRPEAVHEYV |
| | | TPLFVSRAPNRKMSGAHKDTLRSAKRFVKHNEKISVKRVWLTEIKLADLENMVNY |
| | | KNGREIELYEALKARLEAYGGNAKQAFDPKDNPFYKKGGQLVKAVRVEKTQESGV |
| | | LLNKKNAYTIADNGDMVRVDVFCKVDKKGKNQYFIVPIYAWQVAENILPDIDCKG |
| | | YRIDDSYTFCFSLHKYDLIAFQKDEKSKVEFAYYINCDSSNGRFYLAWHDKGSKE |
| | | QQFRISTQNLVLIQKYQVNELGKEIRPCRLKKRPPVR |
| 611 | Amino acid | MDGSGGGSPKKKRKVGGSGGGAAFKPNPINYILGLDIGIASVGWAMVEIDEEENP |
| | sequence for | IRLIDLGVRVFERAEVPKTGDSLAMARRLARSVRRLTRRRAHRLLRARRLLKREG |
| | Nme2Cas9 | VLQAADEDENGLIKSLPNTPWQLRAAALDRKLTPLEWSAVLLHLIKHRGYLSQRK |
| | encoded | NEGETADKELGALLKGVANNAHALQTGDFRTPAELALNKFEKESGHIRNQRGDYS |
| | by mRNA L | HTFSRKDLQAELILLFEKQKEFGNPHVSGGLKEGIETLLMTQRPALSGDAVQKML |
| | | GHCTFEPAEPKAAKNTYTAERFIWLTKLNNLRILEQGSERPLTDTERATLMDEPY |
| | | RKSKLTYAQARKLLGLEDTAFFKGLRYGKDNAEASTLMEMKAYHAISRALEKEGL |
| | | KDKKSPLNLSSELQDEIGTAFSLEKTDEDITGRLKDRVQPEILEALLKHISFDKF |
| | | VQISLKALRRIVPLMEQGKRYDEACAEIYGDHYGKKNTEEKIYLPPIPADEIRNP |
| | | VVLRALSQARKVINGVVRRYGSPARIHIETAREVGKSFKDRKEIEKRQEENRKDR |
| | | EKAAAKFREYFPNFVGEPKSKDILKLRLYEQQHGKCLYSGKEINLVRLNEKGYVE |
| | | IDHALPFSRTWDDSFNNKVLVLGSENQNKGNQTPYEYFNGKDNSREWQEFKARVE |
| | | TSRFPRSKKQRILLQKEDEDGFKECNLNDTRYVNRFLCQFVADHILLTGKGKRRV |
| | | FASNGQITNLLRGFWGLRKVRAENDRHHALDAVVVACSTVAMQQKITRFVRYKEM |
| | | NAFDGKTIDKETGKVLHQKTHFPQPWEFFAQEVMIRVFGKPDGKPEFEEADTPEK |
| | | LRTLLAEKLSSRPEAVHEYVTPLFVSRAPNRKMSGAHKDTLRSAKRFVKHNEKIS |
| | | VKRVWLTEIKLADLENMVNYKNGREIELYEALKARLEAYGGNAKQAFDPKDNPFY |
| | | KKGGQLVKAVRVEKTQESGVLLNKKNAYTIADNGDMVRVDVFCKVDKKGKNQYFI |
| | | VPIYAWQVAENILPDIDCKGYRIDDSYTFCFSLHKYDLIAFQKDEKSKVEFAYYI |
| | | NCDSSNGRFYLAWHDKGSKEQQFRISTQNLVLIQKYQVNELGKEIRPCRLKKRPP |
| | | VR |
| 612 | Amino acid | MDGSGGGSPKKKRKVGGSGGGAAFKPNPINYILGLDIGIASVGWAMVEIDEEENP |
| | sequence for | IRLIDLGVRVFERAEVPKTGDSLAMARRLARSVRRLTRRRAHRLLRARRLLKREG |
| | Nme2Cas9 with | VLQAADFDENGLIKSLPNTPWQLRAAALDRKLTPLEWSAVLLHLIKHRGYLSQRK |
| | HiBiT tag | NEGETADKELGALLKGVANNAHALQTGDFRTPAELALNKFEKESGHIRNQRGDYS |
| | encoded by mRNA | HTFSRKDLQAELILLFEKQKEFGNPHVSGGLKEGIETLLMTQRPALSGDAVQKML |
| | M | GHCTFEPAEPKAAKNTYTAERFIWLTKLNNLRILEQGSERPLTDTERATLMDEPY |
| | | RKSKLTYAQARKLLGLEDTAFFKGLRYGKDNAEASTLMEMKAYHAISRALEKEGL |
| | | KDKKSPLNLSSELQDEIGTAFSLFKTDEDITGRLKDRVQPEILEALLKHISFDKF |
| | | VQISLKALRRIVPLMEQGKRYDEACAEIYGDHYGKKNTEEKIYLPPIPADEIRNP |
| | | VVLRALSQARKVINGVVRRYGSPARIHIETAREVGKSFKDRKEIEKRQEENRKDR |
| | | EKAAAKFREYFPNFVGEPKSKDILKLRLYEQQHGKCLYSGKEINLVRLNEKGYVE |
| | | IDHALPESRTWDDSFNNKVLVLGSENQNKGNQTPYEYENGKDNSREWQEFKARVE |
| | | TSRFPRSKKQRILLQKEDEDGFKECNLNDTRYVNRFLCQFVADHILLTGKGKRRV |
| | | FASNGQITNLLRGFWGLRKVRAENDRHHALDAVVVACSTVAMQQKITRFVRYKEM |
| | | NAFDGKTIDKETGKVLHQKTHEPQPWEFFAQEVMIRVFGKPDGKPEFEEADTPEK |
| | | LRTLLAEKLSSRPEAVHEYVTPLFVSRAPNRKMSGAHKDTLRSAKRFVKHNEKIS |
| | | VKRVWLTEIKLADLENMVNYKNGREIELYEALKARLEAYGGNAKQAFDPKDNPFY |
| | | KKGGQLVKAVRVEKTQESGVLLNKKNAYTIADNGDMVRVDVFCKVDKKGKNQYFI |
| | | VPIYAWQVAENILPDIDCKGYRIDDSYTFCFSLHKYDLIAFQKDEKSKVEFAYYI |
| | | NCDSSNGRFYLAWHDKGSKEQQFRISTQNLVLIQKYQVNELGKEIRPCRLKKRPP |
| | | VRSESATPESVSGWRLEKKIS |
| 613 | Amino acid | MDGSGGGSPKKKRKVGGSGGGAAFKPNPINYILGLDIGIASVGWAMVEIDEEENP |
| | sequence for | IRLIDLGVRVFERAEVPKTGDSLAMARRLARSVRRLTRRRAHRLLRARRLLKREG |
| | Nme2Cas9 | VLQAADFDENGLIKSLPNTPWQLRAAALDRKLTPLEWSAVLLHLIKHRGYLSQRK |
| | encoded | NEGETADKELGALLKGVANNAHALQTGDFRTPAELALNKFEKESGHIRNQRGDYS |
| | by mRNA N | HTFSRKDLQAELILLFEKQKEFGNPHVSGGLKEGIETLLMTQRPALSGDAVQKML |
| | | GHCTFEPAEPKAAKNTYTAERFIWLTKLNNLRILEQGSERPLTDTERATLMDEPY |
| | | RKSKLTYAQARKLLGLEDTAFFKGLRYGKDNAEASTLMEMKAYHAISRALEKEGL |
| | | KDKKSPLNLSSELQDEIGTAFSLEKTDEDITGRLKDRVQPEILEALLKHISFDKF |
| | | VQISLKALRRIVPLMEQGKRYDEACAEIYGDHYGKKNTEEKIYLPPIPADEIRNP |
| | | VVLRALSQARKVINGVVRRYGSPARIHIETAREVGKSFKDRKEIEKRQEENRKDR |
| | | EKAAAKFREYFPNFVGEPKSKDILKLRLYEQQHGKCLYSGKEINLVRLNEKGYVE |
| | | IDHALPFSRTWDDSENNKVLVLGSENQNKGNQTPYEYENGKDNSREWQEFKARVE |
| | | TSRFPRSKKQRILLQKEDEDGFKECNLNDTRYVNRFLCQFVADHILLTGKGKRRV |
| | | FASNGQITNLLRGFWGLRKVRAENDRHHALDAVVVACSTVAMQQKITREVRYKEM |
| | | NAFDGKTIDKETGKVLHQKTHFPQPWEFFAQEVMIRVFGKPDGKPEFEEADTPEK |
| | | LRTLLAEKLSSRPEAVHEYVTPLFVSRAPNRKMSGAHKDTLRSAKRFVKHNEKIS |
| | | VKRVWLTEIKLADLENMVNYKNGREIELYEALKARLEAYGGNAKQAFDPKDNPFY |
| | | KKGGQLVKAVRVEKTQESGVLLNKKNAYTIADNGDMVRVDVFCKVDKKGKNQYFI |
| | | VPIYAWQVAENILPDIDCKGYRIDDSYTFCFSLHKYDLIAFQKDEKSKVEFAYYI |
| | | NCDSSNGRFYLAWHDKGSKEQQFRISTQNLVLIQKYQVNELGKEIRPCRLKKRPP |
| | | VRSGKRTADGSGGGSPAAKKKKLD |
| 614 | Amino acid | MDGSGGGSPKKKRKVEDKRPAATKKAGQAKKKKGGSGGGAAFKPNPINYILGLDI |
| | sequence for | GIASVGWAMVEIDEEENPIRLIDLGVRVFERAEVPKTGDSLAMARRLARSVRRLT |
| | Nme2Cas9 | RRRAHRLLRARRLLKREGVLQAADEDENGLIKSLPNTPWQLRAAALDRKLTPLEW |
| | encoded | SAVLLHLIKHRGYLSQRKNEGETADKELGALLKGVANNAHALQTGDERTPAELAL |
| | by mRNA O | NKFEKESGHIRNQRGDYSHTFSRKDLQAELILLFEKQKEFGNPHVSGGLKEGIET |
| | | LLMTQRPALSGDAVQKMLGHCTFEPAEPKAAKNTYTAERFIWLTKLNNLRILEQG |
| | | SERPLTDTERATLMDEPYRKSKLTYAQARKLLGLEDTAFFKGLRYGKDNAEASTL |
| | | MEMKAYHAISRALEKEGLKDKKSPLNLSSELQDEIGTAFSLFKTDEDITGRLKDR |
| | | VQPEILEALLKHISFDKFVQISLKALRRIVPLMEQGKRYDEACAEIYGDHYGKKN |
| | | TEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCL |
| | | YSGKEINLVRLNEKGYVEIDHALPFSRTWDDSENNKVLVLGSENQNKGNQTPYEY |
| | | ENGKDNSREWQEFKARVETSRFPRSKKQRILLQKFDEDGFKECNLNDTRYVNRFL |
| | | CQFVADHILLTGKGKRRVFASNGQITNLLRGFWGLRKVRAENDRHHALDAVVVAC |
| | | STVAMQQKITRFVRYKEMNAFDGKTIDKETGKVLHQKTHFPQPWEFFAQEVMIRV |
| | | FGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLE |
| | | AYGGNAKQAFDPKDNPFYKKGGQLVKAVRVEKTQESGVLLNKKNAYTIADNGDMV |
| | | RVDVFCKVDKKGKNQYFIVPIYAWQVAENILPDIDCKGYRIDDSYTFCFSLHKYD |
| | | LIAFQKDEKSKVEFAYYINCDSSNGRFYLAWHDKGSKEQQFRISTQNLVLIQKYQ |
| | | VNELGKEIRPCRLKKRPPVR |
| 615 | Amino acid | MDGSGGGSPKKKRKVEDKRPAATKKAGQAKKKKGGSGGGAAFKPNPINYILGLDI |
| | sequence for | GIASVGWAMVEIDEEENPIRLIDLGVRVFERAEVPKTGDSLAMARRLARSVRRLT |
| | Nme2Cas9 with | RRRAHRLLRARRLLKREGVLQAADEDENGLIKSLPNTPWQLRAAALDRKLTPLEW |
| | HiBiT tag | SAVLLHLIKHRGYLSQRKNEGETADKELGALLKGVANNAHALQTGDERTPAELAL |
| | encoded by mRNA | NKFEKESGHIRNQRGDYSHTFSRKDLQAELILLFEKQKEFGNPHVSGGLKEGIET |
| | P | LLMTQRPALSGDAVQKMLGHCTFEPAEPKAAKNTYTAERFIWLTKLNNLRILEQG |
| | | SERPLTDTERATLMDEPYRKSKLTYAQARKLLGLEDTAFFKGLRYGKDNAEASTL |
| | | MEMKAYHAISRALEKEGLKDKKSPLNLSSELQDEIGTAFSLFKTDEDITGRLKDR |
| | | VQPEILEALLKHISFDKFVQISLKALRRIVPLMEQGKRYDEACAEIYGDHYGKKN |
| | | TEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCL |
| | | YSGKEINLVRLNEKGYVEIDHALPESRTWDDSENNKVLVLGSENQNKGNQTPYEY |
| | | ENGKDNSREWQEFKARVETSRFPRSKKQRILLQKFDEDGFKECNLNDTRYVNRFL |
| | | CQFVADHILLTGKGKRRVFASNGQITNLLRGFWGLRKVRAENDRHHALDAVVVAC |
| | | STVAMQQKITRFVRYKEMNAFDGKTIDKETGKVLHQKTHFPQPWEFFAQEVMIRV |
| | | FGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLE |
| | | AYGGNAKQAFDPKDNPFYKKGGQLVKAVRVEKTQESGVLLNKKNAYTIADNGDMV |
| | | RVDVFCKVDKKGKNQYFIVPIYAWQVAENILPDIDCKGYRIDDSYTFCFSLHKYD |
| | | LIAFQKDEKSKVEFAYYINCDSSNGRFYLAWHDKGSKEQQFRISTQNLVLIQKYQ |
| | | VNELGKEIRPCRLKKRPPVRSESATPESVSGWRLFKKIS |
| 616 | Amino acid | MDGSGGGSEDKRPAATKKAGQAKKKKGGSGGGAAFKPNPINYILGLDIGIASVGW |
| | sequence for | AMVEIDEEENPIRLIDLGVRVFERAEVPKTGDSLAMARRLARSVRRLTRRRAHRL |
| | Nme2Cas9 | LRARRLLKREGVLQAADEDENGLIKSLPNTPWQLRAAALDRKLTPLEWSAVLLHL |
| | encoded | IKHRGYLSQRKNEGETADKELGALLKGVANNAHALQTGDFRTPAELALNKFEKES |
| | by mRNA Q | GHIRNQRGDYSHTFSRKDLQAELILLFEKQKEFGNPHVSGGLKEGIETLLMTQRP |
| | | ALSGDAVQKMLGHCTFEPAEPKAAKNTYTAERFIWLTKLNNLRILEQGSERPLTD |
| | | TERATLMDEPYRKSKLTYAQARKLLGLEDTAFFKGLRYGKDNAEASTLMEMKAYH |
| | | AISRALEKEGLKDKKSPLNLSSELQDEIGTAFSLEKTDEDITGRLKDRVQPEILE |
| | | ALLKHISFDKFVQISLKALRRIVPLMEQGKRYDEACAEIYGDHYGKKNTEEKIYL |
| | | PPIPADEIRNPVVLRALSQARKVINGVVRRYGSPARIHIETAREVGKSFKDRKEI |
| | | EKRQEENRKDREKAAAKFREYFPNFVGEPKSKDILKLRLYEQQHGKCLYSGKEIN |
| | | LVRLNEKGYVEIDHALPFSRTWDDSFNNKVLVLGSENQNKGNQTPYEYFNGKDNS |
| | | REWQEFKARVETSRFPRSKKQRILLQKFDEDGEKECNLNDTRYVNRFLCQFVADH |
| | | ILLTGKGKRRVFASNGQITNLLRGFWGLRKVRAENDRHHALDAVVVACSTVAMQQ |
| | | KITRFVRYKEMNAFDGKTIDKETGKVLHQKTHFPQPWEFFAQEVMIRVEGKPDGK |
| | | PEFEEADTPEKLRTLLAEKLSSRPEAVHEYVTPLFVSRAPNRKMSGAHKDTLRSA |
| | | KRFVKHNEKISVKRVWLTEIKLADLENMVNYKNGREIELYEALKARLEAYGGNAK |
| | | QAFDPKDNPFYKKGGQLVKAVRVEKTQESGVLLNKKNAYTIADNGDMVRVDVFCK |
| | | VDKKGKNQYFIVPIYAWQVAENILPDIDCKGYRIDDSYTFCFSLHKYDLIAFQKD |
| | | EKSKVEFAYYINCDSSNGRFYLAWHDKGSKEQQFRISTQNLVLIQKYQVNELGKE |
| | | IRPCRLKKRPPVR |
| 617 | Amino acid | MDGSGGGSPKKKRKVEDKRPAATKKAGQAKKKKGGSGGGEASPASGPRHLMDPHI |
| | sequence for | FTSNENNGIGRHKTYLCYEVERLDNGTSVKMDQHRGFLHNQAKNLLCGFYGRHAE |
| | Nme2Cas9 base | LRFLDLVPSLQLDPAQIYRVTWFISWSPCFSWGCAGEVRAFLQENTHVRLRIFAA |
| | editor encoded | RIYDYDPLYKEALQMLRDAGAQVSIMTYDEFKHCWDTFVDHQGCPFQPWDGLDEH |
| | by mRNA R | SQALSGRLRAILQNQGNSGSETPGTSESATPESAAFKPNPINYILGLAIGIASVG |
| | | WAMVEIDEEENPIRLIDLGVRVFERAEVPKTGDSLAMARRLARSVRRLTRRRAHR |
| | | LLRARRLLKREGVLQAADFDENGLIKSLPNTPWQLRAAALDRKLTPLEWSAVLLH |
| | | LIKHRGYLSQRKNEGETADKELGALLKGVANNAHALQTGDFRTPAELALNKFEKE |
| | | SGHIRNQRGDYSHTFSRKDLQAELILLFEKQKEFGNPHVSGGLKEGIETLLMTQR |
| | | PALSGDAVQKMLGHCTFEPAEPKAAKNTYTAERFIWLTKLNNLRILEQGSERPLT |
| | | DTERATLMDEPYRKSKLTYAQARKLLGLEDTAFFKGLRYGKDNAEASTLMEMKAY |
| | | HAISRALEKEGLKDKKSPLNLSSELQDEIGTAFSLEKTDEDITGRLKDRVQPEIL |
| | | EALLKHISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSFKDRKE |
| | | IEKRQEENRKDREKAAAKFREYFPNFVGEPKSKDILKLRLYEQQHGKCLYSGKEI |
| | | NLVRLNEKGYVEIDHALPESRTWDDSFNNKVLVLGSENQNKGNQTPYEYENGKDN |
| | | SREWQEFKARVETSRFPRSKKQRILLQKFDEDGEKECNLNDTRYVNRFLCQFVAD |
| | | HILLTGKGKRRVFASNGQITNLLRGFWGLRKVRAENDRHHALDAVVVACSTVAMQ |
| | | QKITRFVRYKEMNAFDGKTIDKETGKVLHQKTHFPQPWEFFAQEVMIRVFGKPDG |
| | | KPEFEEADTPEKLRTLLAEKLSSRPEAVHEYVTPLFVSRAPNRKMSGAHKDTLRS |
| | | AKRFVKHNEKISVKRVWLTEIKLADLENMVNYKNGREIELYEALKARLEAYGGNA |
| | | KQAFDPKDNPFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKYDLIAFQK |
| | | DEKSKVEFAYYINCDSSNGRFYLAWHDKGSKEQQFRISTQNLVLIQKYQVNELGK |
| | | EIRPCRLKKRPPVRSGKRTADGSEFESPKKKRKVE |
| 618 | Amino acid | MDGSGGGSPKKKRKVEDKRPAATKKAGQAKKKKGGSGGGEASPASGPRHLMDPHI |
| | sequence for | FTSNENNGIGRHKTYLCYEVERLDNGTSVKMDQHRGFLHNQAKNLLCGFYGRHAE |
| | Nme2Cas9 base | LRFLDLVPSLQLDPAQIYRVTWFISWSPCESWGCAGEVRAFLQENTHVRLRIFAA |
| | editor encoded | RIYDYDPLYKEALQMLRDAGAQVSIMTYDEFKHCWDTFVDHQGCPFQPWDGLDEH |
| | by mRNA S | SQALSGRLRAILQNQGNSGSETPGTSESATPESAAFKPNPINYILGLAIGIASVG |
| | | WAMVEIDEEENPIRLIDLGVRVFERAEVPKTGDSLAMARRLARSVRRLTRRRAHR |
| | | LLRARRLLKREGVLQAADFDENGLIKSLPNTPWQLRAAALDRKLTPLEWSAVLLH |
| | | LIKHRGYLSQRKNEGETADKELGALLKGVANNAHALQTGDFRTPAELALNKFEKE |
| | | SGHIRNQRGDYSHTFSRKDLQAELILLFEKQKEFGNPHVSGGLKEGIETLLMTQR |
| | | PALSGDAVQKMLGHCTFEPAEPKAAKNTYTAERFIWLTKLNNLRILEQGSERPLT |
| | | DTERATLMDEPYRKSKLTYAQARKLLGLEDTAFFKGLRYGKDNAEASTLMEMKAY |
| | | HAISRALEKEGLKDKKSPLNLSSELQDEIGTAFSLFKTDEDITGRLKDRVQPEIL |
| | | EALLKHISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSFKDRKE |
| | | IEKRQEENRKDREKAAAKFREYFPNFVGEPKSKDILKLRLYEQQHGKCLYSGKEI |
| | | NLVRLNEKGYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRFLCQFVAD |
| | | HILLTGKGKRRVFASNGQITNLLRGFWGLRKVRAENDRHHALDAVVVACSTVAMQ |
| | | QKITRFVRYKEMNAFDGKTIDKETGKVLHQKTHFPQPWEFFAQEVMIRVFGKPDG |
| | | KPEFEEADTPEKLRTLLAEKLSSRPEAVHEYVTPLFVSRAPNRKMSGAHKDTLRS |
| | | AKRFVKHNEKISVKRVWLTEIKLADLENMVNYKNGREIELYEALKARLEAYGGNA |
| | | KQAFDPKDNPFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKYDLIAFQK |
| | | DEKSKVEFAYYINCDSSNGRFYLAWHDKGSKEQQFRISTQNLVLIQKYQVNELGK |
| | | EIRPCRLKKRPPVR |
| 619 | Amino acid | MEASPASGPRHLMDPHIFTSNENNGIGRHKTYLCYEVERLDNGTSVKMDQHRGEL |
| | sequence for | HNQAKNLLCGFYGRHAELRFLDLVPSLQLDPAQIYRVTWFISWSPCFSWGCAGEV |
| | Nme2Cas9 base | RAFLQENTHVRLRIFAARIYDYDPLYKEALQMLRDAGAQVSIMTYDEFKHCWDTF |
| | editor encoded | VDHQGCPFQPWDGLDEHSQALSGRLRAILQNQGNSGSETPGTSESATPESAAFKP |
| | by mRNA T | NPINYILGLAIGIASVGWAMVEIDEEENPIRLIDLGVRVFERAEVPKTGDSLAMA |
| | | RRLARSVRRLTRRRAHRLLRARRLLKREGVLQAADFDENGLIKSLPNTPWQLRAA |
| | | ALDRKLTPLEWSAVLLHLIKHRGYLSQRKNEGETADKELGALLKGVANNAHALQT |
| | | GDFRTPAELALNKFEKESGHIRNQRGDYSHTFSRKDLQAELILLFEKQKEFGNPH |
| | | VSGGLKEGIETLLMTQRPALSGDAVQKMLGHCTFEPAEPKAAKNTYTAERFIWLT |
| | | KLNNLRILEQGSERPLTDTERATLMDEPYRKSKLTYAQARKLLGLEDTAFFKGLR |
| | | YGKDNAEASTLMEMKAYHAISRALEKEGLKDKKSPLNLSSELQDEIGTAFSLFKT |
| | | DEDITGRLKDRVQPEILEALLKHISFDKFVQISLKALRRIVPLMEQGKRYDEACA |
| | | EIYGDHYGKKNTEEKIYLPPIPADEIRNPVVLRALSQARKVINGVVRRYGSPARI |
| | | HIETAREVGKSFKDRKEIEKRQEENRKDREKAAAKFREYEPNFVGEPKSKDILKL |
| | | RLYEQQHGKCLYSGKEINLVRLNEKGYVEIDHALPFSRTWDDSFNNKVLVLGSEN |
| | | QNKGNQTPYEYENGKDNSREWQEFKARVETSRFPRSKKQRILLQKFDEDGFKECN |
| | | LNDTRYVNRFLCQFVADHILLTGKGKRRVFASNGQITNLLRGFWGLRKVRAENDR |
| | | HHALDAVVVACSTVAMQQKITRFVRYKEMNAFDGKTIDKETGKVLHQKTHFPQPW |
| | | EFFAQEVMIRVFGKPDGKPEFEEADTPEKLRTLLAEKLSSRPEAVHEYVTPLFVS |
| | | RAPNRKMSGAHKDTLRSAKRFVKHNEKISVKRVWLTEIKLADLENMVNYKNGREI |
| | | ELYEALKARLEAYGGNAKQAFDPKDNPFYKKGGQLVKAVRVEKTQESGVLLNKKN |
| | | AYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRIS |
| | | TQNLVLIQKYQVNELGKEIRPCRLKKRPPVRSGKRTADGSEFESPKKKRKVE |
| 620 | Amino acid | MKLGSIEFIKVNKGSGSGSGAPESATESGGTSTESEGSAGTSTESEGSAGSAGST |
| | sequence for | STEEGTSTESEGSAGTSTESEGSAGTSESATESGGTSTESEGSSSTGAAFKPNPI |
| | Nme2Cas9 | NYILGLDIGIASVGWAMVEIDEEENPIRLIDLGVRVFERAEVPKTGDSLAMARRL |
| | encoded | ARSVRRLTRRRAHRLLRARRLLKREGVLQAADFDENGLIKSLPNTPWQLRAAALD |
| | by mRNA U | RKLTPLEWSAVLLHLIKHRGYLSQRKNEGETADKELGALLKGVANNAHALQTGDF |
| | | RTPAELALNKFEKESGHIRNQRGDYSHTFSRKDLQAELILLFEKQKEFGNPHVSG |
| | | GLKEGIETLLMTQRPALSGDAVQKMLGHCTFEPAEPKAAKNTYTAERFIWLTKLN |
| | | NLRILEQGSERPLTDTERATLMDEPYRKSKLTYAQARKLLGLEDTAFFKGLRYGK |
| | | DNAEASTLMEMKAYHAISRALEKEGLKDKKSPLNLSSELQDEIGTAFSLEKTDED |
| | | ITGRLKDRVQPEILEALLKHISFDKFVQISLKALRRIVPLMEQGKRYDEACAEIY |
| | | GDHYGKKNTEEKIYLPPIPADEIRNPVVLRALSQARKVINGVVRRYGSPARIHIE |
| | | TAREVGKSFKDRKEIEKRQEENRKDREKAAAKFREYFPNFVGEPKSKDILKLRLY |
| | | EQQHGKCLYSGKEINLVRLNEKGYVEIDHALPFSRTWDDSFNNKVLVLGSENQNK |
| | | GNQTPYEYENGKDNSREWQEFKARVETSRFPRSKKQRILLQKFDEDGFKECNLND |
| | | TRYVNRFLCQFVADHILLTGKGKRRVFASNGQITNLLRGFWGLRKVRAENDRHHA |
| | | LDAVVVACSTVAMQQKITREVRYKEMNAFDGKTIDKETGKVLHQKTHFPQPWEFF |
| | | AQEVMIRVFGKPDGKPEFEEADTPEKLRTLLAEKLSSRPEAVHEYVTPLFVSRAP |
| | | NRKMSGAHKDTLRSAKRFVKHNEKISVKRVWLTEIKLADLENMVNYKNGREIELY |
| | | EALKARLEAYGGNAKQAFDPKDNPFYKKGGQLVKAVRVEKTQESGVLLNKKNAYT |
| | | IADNGDMVRVDVFCKVDKKGKNQYFIVPIYAWQVAENILPDIDCKGYRIDDSYTF |
| | | CFSLHKYDLIAFQKDEKSKVEFAYYINCDSSNGRFYLAWHDKGSKEQQFRISTQN |
| | | LVLIQKYQVNELGKEIRPCRLKKRPPVRSGKRTADGSEFESPKKKRKVE |
| 621 | mRNA B encoding | GGGAAGCUCAGAAUAAACGCUCAACUUUGGCCGGAUCUGCCACCAUGACCGGUGC |
| | Nme2Cas 9 | CGCCUUCAAGCCCAACCCCAUCAACUACAUCCUGGGCCUGGACAUCGGCAUCGCC |
| | | UCCGUGGGCUGGGCCAUGGUGGAGAUCGACGAGGAGGAGAACCCCAUCCGGCUGA |
| | | UCGACCUGGGCGUGCGGGUGUUCGAGCGGGCCGAGGUGCCCAAGACCGGCGACUC |
| | | CCUGGCCAUGGCCCGGCGGCUGGCCCGGUCCGUGCGGCGGCUGACCCGGCGGCGG |
| | | GCCCACCGGCUGCUGCGGGCCCGGCGGCUGCUGAAGCGGGAGGGCGUGCUGCAGG |
| | | CCGCCGACUUCGACGAGAACGGCCUGAUCAAGUCCCUGCCCAACACCCCCUGGCA |
| | | GCUGCGGGCCGCCGCCCUGGACCGGAAGCUGACCCCCCUGGAGUGGUCCGCCGUG |
| | | CUGCUGCACCUGAUCAAGCACCGGGGCUACCUGUCCCAGCGGAAGAACGAGGGCG |
| | | AGACCGCCGACAAGGAGCUGGGCGCCCUGCUGAAGGGCGUGGCCAACAACGCCCA |
| | | CGCCCUGCAGACCGGCGACUUCCGGACCCCCGCCGAGCUGGCCCUGAACAAGUUC |
| | | GAGAAGGAGUCCGGCCACAUCCGGAACCAGCGGGGCGACUACUCCCACACCUUCU |
| | | CCCGGAAGGACCUGCAGGCCGAGCUGAUCCUGCUGUUCGAGAAGCAGAAGGAGUU |
| | | CGGCAACCCCCACGUGUCCGGCGGCCUGAAGGAGGGCAUCGAGACCCUGCUGAUG |
| | | ACCCAGCGGCCCGCCCUGUCCGGCGACGCCGUGCAGAAGAUGCUGGGCCACUGCA |
| | | CCUUCGAGCCCGCCGAGCCCAAGGCCGCCAAGAACACCUACACCGCCGAGCGGUU |
| | | CAUCUGGCUGACCAAGCUGAACAACCUGCGGAUCCUGGAGCAGGGCUCCGAGCGG |
| | | CCCCUGACCGACACCGAGCGGGCCACCCUGAUGGACGAGCCCUACCGGAAGUCCA |
| | | AGCUGACCUACGCCCAGGCCCGGAAGCUGCUGGGCCUGGAGGACACCGCCUUCUU |
| | | CAAGGGCCUGCGGUACGGCAAGGACAACGCCGAGGCCUCCACCCUGAUGGAGAUG |
| | | AAGGCCUACCACGCCAUCUCCCGGGCCCUGGAGAAGGAGGGCCUGAAGGACAAGA |
| | | AGUCCCCCCUGAACCUGUCCUCCGAGCUGCAGGACGAGAUCGGCACCGCCUUCUC |
| | | CCUGUUCAAGACCGACGAGGACAUCACCGGCCGGCUGAAGGACCGGGUGCAGCCC |
| | | GAGAUCCUGGAGGCCCUGCUGAAGCACAUCUCCUUCGACAAGUUCGUGCAGAUCU |
| | | CCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAG |
| | | AAGAUCUACCUGCCCCCCAUCCCCGCCGACGAGAUCCGGAACCCCGUGGUGCUGC |
| | | GGGCCCUGUCCCAGGCCCGGAAGGUGAUCAACGGCGUGGUGCGGCGGUACGGCUC |
| | | CCCCGCCCGGAUCCACAUCGAGACCGCCCGGGAGGUGGGCAAGUCCUUCAAGGAC |
| | | CGGAAGGAGAUCGAGAAGCGGCAGGAGGAGAACCGGAAGGACCGGGAGAAGGCCG |
| | | CCGCCAAGUUCCGGGAGUACUUCCCCAACUUCGUGGGCGAGCCCAAGUCCAAGGA |
| | | CAUCCUGAAGCUGCGGCUGUACGAGCAGCAGCACGGCAAGUGCCUGUACUCCGGC |
| | | AAGGAGAUCAACCUGGUGCGGCUGAACGAGAAGGGCUACGUGGAGAUCGACCACG |
| | | CCCUGCCCUUCUCCCGGACCUGGGACGACUCCUUCAACAACAAGGUGCUGGUGCU |
| | | GGGCUCCGAGAACCAGAACAAGGGCAACCAGACCCCCUACGAGUACUUCAACGGC |
| | | AAGGACAACUCCCGGGAGUGGCAGGAGUUCAAGGCCCGGGUGGAGACCUCCCGGU |
| | | UCCCCCGGUCCAAGAAGCAGCGGAUCCUGCUGCAGAAGUUCGACGAGGACGGCUU |
| | | CAAGGAGUGCAACCUGAACGACACCCGGUACGUGAACCGCUUCCUGUGCCAGUUC |
| | | GUGGCCGACCACAUCCUGCUGACCGGCAAGGGCAAGCGGCGGGUGUUCGCCUCCA |
| | | ACGGCCAGAUCACCAACCUGCUGCGGGGCUUCUGGGGCCUGCGGAAGGUGCGGGC |
| | | CGAGAACGACCGGCACCACGCCCUGGACGCCGUGGUGGUGGCCUGCUCCACCGUG |
| | | GCCAUGCAGCAGAAGAUCACCCGGUUCGUGCGGUACAAGGAGAUGAACGCCUUCG |
| | | ACGGCAAGACCAUCGACAAGGAGACCGGCAAGGUGCUGCACCAGAAGACCCACUU |
| | | CCCCCAGCCCUGGGAGUUCUUCGCCCAGGAGGUGAUGAUCCGGGUGUUCGGCAAG |
| | | CCCGACGGCAAGCCCGAGUUCGAGGAGGCCGACACCCCCGAGAAGCUGCGGACCC |
| | | UGCUGGCCGAGAAGCUGUCCUCCCGGCCCGAGGCCGUGCACGAGUACGUGACCCC |
| | | CCUGUUCGUGUCCCGGGCCCCCAACCGGAAGAUGUCCGGCGCCCACAAGGACACC |
| | | CUGCGGUCCGCCAAGCGGUUCGUGAAGCACAACGAGAAGAUCUCCGUGAAGCGGG |
| | | UGUGGCUGACCGAGAUCAAGCUGGCCGACCUGGAGAACAUGGUGAACUACAAGAA |
| | | CGGCCGGGAGAUCGAGCUGUACGAGGCCCUGAAGGCCCGGCUGGAGGCCUACGGC |
| | | GGCAACGCCAAGCAGGCCUUCGACCCCAAGGACAACCCCUUCUACAAGAAGGGCG |
| | | GCCAGCUGGUGAAGGCCGUGCGGGUGGAGAAGACCCAGGAGUCCGGCGUGCUGCU |
| | | GAACAAGAAGAACGCCUACACCAUCGCCGACAACGGCGACAUGGUGCGGGUGGAC |
| | | GUGUUCUGCAAGGUGGACAAGAAGGGCAAGAACCAGUACUUCAUCGUGCCCAUCU |
| | | ACGCCUGGCAGGUGGCCGAGAACAUCCUGCCCGACAUCGACUGCAAGGGCUACCG |
| | | GAUCGACGACUCCUACACCUUCUGCUUCUCCCUGCACAAGUACGACCUGAUCGCC |
| | | UUCCAGAAGGACGAGAAGUCCAAGGUGGAGUUCGCCUACUACAUCAACUGCGACU |
| | | CCUCCAACGGCCGGUUCUACCUGGCCUGGCACGACAAGGGCUCCAAGGAGCAGCA |
| | | GUUCCGGAUCUCCACCCAGAACCUGGUGCUGAUCCAGAAGUACCAGGUGAACGAG |
| | | CUGGGCAAGGAGAUCCGGCCCUGCCGGCUGAAGAAGCGGCCCCCCGUGCGGUCCG |
| | | GAAAGCGGACCGCCGACGGCUCCGAGUUCGAGUCCCCCAAGAAGAAGCGGAAGGU |
| | | GGAGUAGCUAGCACCAGCCUCAAGAACACCCGAAUGGAGUCUCUAAGCUACAUAA |
| | | UACCAACUUACACUUUACAAAAUGUUGUCCCCCAAAAUGUAGCCAUUCGUAUCUG |
| | | CUCCUAAUAAAAAGAAAGUUUCUUCACAUUCUCUCGAGAAAAAAAAAAAAUGGAA |
| | | AAAAAAAAAACGGAAAAAAAAAAAAGGUAAAAAAAAAAAAUAUAAAAAAAAAAAA |
| | | CAUAAAAAAAAAAAACGAAAAAAAAAAAACGUAAAAAAAAAAAACUCAAAAAAAA |
| | | AAAAGAUAAAAAAAAAAAACCUAAAAAAAAAAAAUGUAAAAAAAAAAAAUCUAG |
| 622 | mRNA C encoding | GGGAAGCUCAGAAUAAACGCUCAACUUUGGCCGGAUCUGCCACCAUGACCGGUGC |
| | Nme2Cas 9 | CGCCUUCAAGCCCAACCCCAUCAACUACAUCCUGGGCCUGGACAUCGGCAUCGCC |
| | | UCCGUGGGCUGGGCCAUGGUGGAGAUCGACGAGGAGGAGAACCCCAUCCGGCUGA |
| | | UCGACCUGGGCGUGCGGGUGUUCGAGCGGGCCGAGGUGCCCAAGACCGGCGACUC |
| | | CCUGGCCAUGGCCCGGCGGCUGGCCCGGUCCGUGCGGCGGCUGACCCGGCGGCGG |
| | | GCCCACCGGCUGCUGCGGGCCCGGCGGCUGCUGAAGCGGGAGGGCGUGCUGCAGG |
| | | CCGCCGACUUCGACGAGAACGGCCUGAUCAAGUCCCUGCCCAACACCCCCUGGCA |
| | | GCUGCGGGCCGCCGCCCUGGACCGGAAGCUGACCCCCCUGGAGUGGUCCGCCGUG |
| | | CUGCUGCACCUGAUCAAGCACCGGGGCUACCUGUCCCAGCGGAAGAACGAGGGCG |
| | | AGACCGCCGACAAGGAGCUGGGCGCCCUGCUGAAGGGCGUGGCCAACAACGCCCA |
| | | CGCCCUGCAGACCGGCGACUUCCGGACCCCCGCCGAGCUGGCCCUGAACAAGUUC |
| | | GAGAAGGAGUCCGGCCACAUCCGGAACCAGCGGGGCGACUACUCCCACACCUUCU |
| | | CCCGGAAGGACCUGCAGGCCGAGCUGAUCCUGCUGUUCGAGAAGCAGAAGGAGUU |
| | | CGGCAACCCCCACGUGUCCGGCGGCCUGAAGGAGGGCAUCGAGACCCUGCUGAUG |
| | | ACCCAGCGGCCCGCCCUGUCCGGCGACGCCGUGCAGAAGAUGCUGGGCCACUGCA |
| | | CCUUCGAGCCCGCCGAGCCCAAGGCCGCCAAGAACACCUACACCGCCGAGCGGUU |
| | | CAUCUGGCUGACCAAGCUGAACAACCUGCGGAUCCUGGAGCAGGGCUCCGAGCGG |
| | | CCCCUGACCGACACCGAGCGGGCCACCCUGAUGGACGAGCCCUACCGGAAGUCCA |
| | | AGCUGACCUACGCCCAGGCCCGGAAGCUGCUGGGCCUGGAGGACACCGCCUUCUU |
| | | CAAGGGCCUGCGGUACGGCAAGGACAACGCCGAGGCCUCCACCCUGAUGGAGAUG |
| | | AAGGCCUACCACGCCAUCUCCCGGGCCCUGGAGAAGGAGGGCCUGAAGGACAAGA |
| | | AGUCCCCCCUGAACCUGUCCUCCGAGCUGCAGGACGAGAUCGGCACCGCCUUCUC |
| | | CCUGUUCAAGACCGACGAGGACAUCACCGGCCGGCUGAAGGACCGGGUGCAGCCC |
| | | GAGAUCCUGGAGGCCCUGCUGAAGCACAUCUCCUUCGACAAGUUCGUGCAGAUCU |
| | | CCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAG |
| | | AAGAUCUACCUGCCCCCCAUCCCCGCCGACGAGAUCCGGAACCCCGUGGUGCUGC |
| | | GGGCCCUGUCCCAGGCCCGGAAGGUGAUCAACGGCGUGGUGCGGCGGUACGGCUC |
| | | CCCCGCCCGGAUCCACAUCGAGACCGCCCGGGAGGUGGGCAAGUCCUUCAAGGAC |
| | | CGGAAGGAGAUCGAGAAGCGGCAGGAGGAGAACCGGAAGGACCGGGAGAAGGCCG |
| | | CCGCCAAGUUCCGGGAGUACUUCCCCAACUUCGUGGGCGAGCCCAAGUCCAAGGA |
| | | CAUCCUGAAGCUGCGGCUGUACGAGCAGCAGCACGGCAAGUGCCUGUACUCCGGC |
| | | AAGGAGAUCAACCUGGUGCGGCUGAACGAGAAGGGCUACGUGGAGAUCGACCACG |
| | | CCCUGCCCUUCUCCCGGACCUGGGACGACUCCUUCAACAACAAGGUGCUGGUGCU |
| | | GGGCUCCGAGAACCAGAACAAGGGCAACCAGACCCCCUACGAGUACUUCAACGGC |
| | | AAGGACAACUCCCGGGAGUGGCAGGAGUUCAAGGCCCGGGUGGAGACCUCCCGGU |
| | | UCCCCCGGUCCAAGAAGCAGCGGAUCCUGCUGCAGAAGUUCGACGAGGACGGCUU |
| | | CAAGGAGUGCAACCUGAACGACACCCGGUACGUGAACCGCUUCCUGUGCCAGUUC |
| | | GUGGCCGACCACAUCCUGCUGACCGGCAAGGGCAAGCGGCGGGUGUUCGCCUCCA |
| | | ACGGCCAGAUCACCAACCUGCUGCGGGGCUUCUGGGGCCUGCGGAAGGUGCGGGC |
| | | CGAGAACGACCGGCACCACGCCCUGGACGCCGUGGUGGUGGCCUGCUCCACCGUG |
| | | GCCAUGCAGCAGAAGAUCACCCGGUUCGUGCGGUACAAGGAGAUGAACGCCUUCG |
| | | ACGGCAAGACCAUCGACAAGGAGACCGGCAAGGUGCUGCACCAGAAGACCCACUU |
| | | CCCCCAGCCCUGGGAGUUCUUCGCCCAGGAGGUGAUGAUCCGGGUGUUCGGCAAG |
| | | CCCGACGGCAAGCCCGAGUUCGAGGAGGCCGACACCCCCGAGAAGCUGCGGACCC |
| | | UGCUGGCCGAGAAGCUGUCCUCCCGGCCCGAGGCCGUGCACGAGUACGUGACCCC |
| | | CCUGUUCGUGUCCCGGGCCCCCAACCGGAAGAUGUCCGGCGCCCACAAGGACACC |
| | | CUGCGGUCCGCCAAGCGGUUCGUGAAGCACAACGAGAAGAUCUCCGUGAAGCGGG |
| | | UGUGGCUGACCGAGAUCAAGCUGGCCGACCUGGAGAACAUGGUGAACUACAAGAA |
| | | CGGCCGGGAGAUCGAGCUGUACGAGGCCCUGAAGGCCCGGCUGGAGGCCUACGGC |
| | | GGCAACGCCAAGCAGGCCUUCGACCCCAAGGACAACCCCUUCUACAAGAAGGGCG |
| | | GCCAGCUGGUGAAGGCCGUGCGGGUGGAGAAGACCCAGGAGUCCGGCGUGCUGCU |
| | | GAACAAGAAGAACGCCUACACCAUCGCCGACAACGGCGACAUGGUGCGGGUGGAC |
| | | GUGUUCUGCAAGGUGGACAAGAAGGGCAAGAACCAGUACUUCAUCGUGCCCAUCU |
| | | ACGCCUGGCAGGUGGCCGAGAACAUCCUGCCCGACAUCGACUGCAAGGGCUACCG |
| | | GAUCGACGACUCCUACACCUUCUGCUUCUCCCUGCACAAGUACGACCUGAUCGCC |
| | | UUCCAGAAGGACGAGAAGUCCAAGGUGGAGUUCGCCUACUACAUCAACUGCGACU |
| | | CCUCCAACGGCCGGUUCUACCUGGCCUGGCACGACAAGGGCUCCAAGGAGCAGCA |
| | | GUUCCGGAUCUCCACCCAGAACCUGGUGCUGAUCCAGAAGUACCAGGUGAACGAG |
| | | CUGGGCAAGGAGAUCCGGCCCUGCCGGCUGAAGAAGCGGCCCCCCGUGCGGUCCG |
| | | GAAAGCGGACCGCCGACGGCUCCGAGUUCGAGUCCCCCAAGAAGAAGCGGAAGGU |
| | | GGAGUAGCUAGCACCAGCCUCAAGAACACCCGAAUGGAGUCUCUAAGCUACAUAA |
| | | UACCAACUUACACUUUACAAAAUGUUGUCCCCCAAAAUGUAGCCAUUCGUAUCUG |
| | | CUCCUAAUAAAAAGAAAGUUUCUUCACAUUCUCUCGAGAAAAAAAAAAAAUGGAA |
| | | AAAAAAAAAACGGAAAAAAAAAAAAGGUAAAAAAAAAAAAUAUAAAAAAAAAAAA |
| | | CAUAAAAAAAAAAAACGAAAAAAAAAAAACGUAAAAAAAAAAAACUCAAAAAAAA |
| | | AAAAGAUAAAAAAAAAAAACCUAAAAAAAAAAAAUGUAAAAAAAAAAAAGGGAAA |
| | | AAAAAAAAACGCAAAAAAAAAAAACACAAAAAAAAAAAAUGCAAAAAAAAAAAAU |
| | | CGAAAAAAAAAAAAUCUAAAAAAAAAAAACGAAAAAAAAAAAACCCAAAAAAAAA |
| | | AAAGACAAAAAAAAAAAAUAGAAAAAAAAAAAAGUUAAAAAA |
| 623 | mRNA D encoding | GGGAAGCUCAGAAUAAACGCUCAACUUUGGCCGGAUCUGCCACCAUGACCGGUGC |
| | Nme2Cas9 | CGCCUUCAAGCCCAACCCCAUCAACUACAUCCUGGGCCUGGACAUCGGCAUCGCC |
| | | UCCGUGGGCUGGGCCAUGGUGGAGAUCGACGAGGAGGAGAACCCCAUCCGGCUGA |
| | | UCGACCUGGGCGUGCGGGUGUUCGAGCGGGCCGAGGUGCCCAAGACCGGCGACUC |
| | | CCUGGCCAUGGCCCGGCGGCUGGCCCGGUCCGUGCGGCGGCUGACCCGGCGGCGG |
| | | GCCCACCGGCUGCUGCGGGCCCGGCGGCUGCUGAAGCGGGAGGGCGUGCUGCAGG |
| | | CCGCCGACUUCGACGAGAACGGCCUGAUCAAGUCCCUGCCCAACACCCCCUGGCA |
| | | GCUGCGGGCCGCCGCCCUGGACCGGAAGCUGACCCCCCUGGAGUGGUCCGCCGUG |
| | | CUGCUGCACCUGAUCAAGCACCGGGGCUACCUGUCCCAGCGGAAGAACGAGGGCG |
| | | AGACCGCCGACAAGGAGCUGGGCGCCCUGCUGAAGGGCGUGGCCAACAACGCCCA |
| | | CGCCCUGCAGACCGGCGACUUCCGGACCCCCGCCGAGCUGGCCCUGAACAAGUUC |
| | | GAGAAGGAGUCCGGCCACAUCCGGAACCAGCGGGGCGACUACUCCCACACCUUCU |
| | | CCCGGAAGGACCUGCAGGCCGAGCUGAUCCUGCUGUUCGAGAAGCAGAAGGAGUU |
| | | CGGCAACCCCCACGUGUCCGGCGGCCUGAAGGAGGGCAUCGAGACCCUGCUGAUG |
| | | ACCCAGCGGCCCGCCCUGUCCGGCGACGCCGUGCAGAAGAUGCUGGGCCACUGCA |
| | | CCUUCGAGCCCGCCGAGCCCAAGGCCGCCAAGAACACCUACACCGCCGAGCGGUU |
| | | CAUCUGGCUGACCAAGCUGAACAACCUGCGGAUCCUGGAGCAGGGCUCCGAGCGG |
| | | CCCCUGACCGACACCGAGCGGGCCACCCUGAUGGACGAGCCCUACCGGAAGUCCA |
| | | AGCUGACCUACGCCCAGGCCCGGAAGCUGCUGGGCCUGGAGGACACCGCCUUCUU |
| | | CAAGGGCCUGCGGUACGGCAAGGACAACGCCGAGGCCUCCACCCUGAUGGAGAUG |
| | | AAGGCCUACCACGCCAUCUCCCGGGCCCUGGAGAAGGAGGGCCUGAAGGACAAGA |
| | | AGUCCCCCCUGAACCUGUCCUCCGAGCUGCAGGACGAGAUCGGCACCGCCUUCUC |
| | | CCUGUUCAAGACCGACGAGGACAUCACCGGCCGGCUGAAGGACCGGGUGCAGCCC |
| | | GAGAUCCUGGAGGCCCUGCUGAAGCACAUCUCCUUCGACAAGUUCGUGCAGAUCU |
| | | CCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAG |
| | | AAGAUCUACCUGCCCCCCAUCCCCGCCGACGAGAUCCGGAACCCCGUGGUGCUGC |
| | | GGGCCCUGUCCCAGGCCCGGAAGGUGAUCAACGGCGUGGUGCGGCGGUACGGCUC |
| | | CCCCGCCCGGAUCCACAUCGAGACCGCCCGGGAGGUGGGCAAGUCCUUCAAGGAC |
| | | CGGAAGGAGAUCGAGAAGCGGCAGGAGGAGAACCGGAAGGACCGGGAGAAGGCCG |
| | | CCGCCAAGUUCCGGGAGUACUUCCCCAACUUCGUGGGCGAGCCCAAGUCCAAGGA |
| | | CAUCCUGAAGCUGCGGCUGUACGAGCAGCAGCACGGCAAGUGCCUGUACUCCGGC |
| | | AAGGAGAUCAACCUGGUGCGGCUGAACGAGAAGGGCUACGUGGAGAUCGACCACG |
| | | CCCUGCCCUUCUCCCGGACCUGGGACGACUCCUUCAACAACAAGGUGCUGGUGCU |
| | | GGGCUCCGAGAACCAGAACAAGGGCAACCAGACCCCCUACGAGUACUUCAACGGC |
| | | AAGGACAACUCCCGGGAGUGGCAGGAGUUCAAGGCCCGGGUGGAGACCUCCCGGU |
| | | UCCCCCGGUCCAAGAAGCAGCGGAUCCUGCUGCAGAAGUUCGACGAGGACGGCUU |
| | | CAAGGAGUGCAACCUGAACGACACCCGGUACGUGAACCGCUUCCUGUGCCAGUUC |
| | | GUGGCCGACCACAUCCUGCUGACCGGCAAGGGCAAGCGGCGGGUGUUCGCCUCCA |
| | | ACGGCCAGAUCACCAACCUGCUGCGGGGCUUCUGGGGCCUGCGGAAGGUGCGGGC |
| | | CGAGAACGACCGGCACCACGCCCUGGACGCCGUGGUGGUGGCCUGCUCCACCGUG |
| | | GCCAUGCAGCAGAAGAUCACCCGGUUCGUGCGGUACAAGGAGAUGAACGCCUUCG |
| | | ACGGCAAGACCAUCGACAAGGAGACCGGCAAGGUGCUGCACCAGAAGACCCACUU |
| | | CCCCCAGCCCUGGGAGUUCUUCGCCCAGGAGGUGAUGAUCCGGGUGUUCGGCAAG |
| | | CCCGACGGCAAGCCCGAGUUCGAGGAGGCCGACACCCCCGAGAAGCUGCGGACCC |
| | | UGCUGGCCGAGAAGCUGUCCUCCCGGCCCGAGGCCGUGCACGAGUACGUGACCCC |
| | | CCUGUUCGUGUCCCGGGCCCCCAACCGGAAGAUGUCCGGCGCCCACAAGGACACC |
| | | CUGCGGUCCGCCAAGCGGUUCGUGAAGCACAACGAGAAGAUCUCCGUGAAGCGGG |
| | | UGUGGCUGACCGAGAUCAAGCUGGCCGACCUGGAGAACAUGGUGAACUACAAGAA |
| | | CGGCCGGGAGAUCGAGCUGUACGAGGCCCUGAAGGCCCGGCUGGAGGCCUACGGC |
| | | GGCAACGCCAAGCAGGCCUUCGACCCCAAGGACAACCCCUUCUACAAGAAGGGCG |
| | | GCCAGCUGGUGAAGGCCGUGCGGGUGGAGAAGACCCAGGAGUCCGGCGUGCUGCU |
| | | GAACAAGAAGAACGCCUACACCAUCGCCGACAACGGCGACAUGGUGCGGGUGGAC |
| | | GUGUUCUGCAAGGUGGACAAGAAGGGCAAGAACCAGUACUUCAUCGUGCCCAUCU |
| | | ACGCCUGGCAGGUGGCCGAGAACAUCCUGCCCGACAUCGACUGCAAGGGCUACCG |
| | | GAUCGACGACUCCUACACCUUCUGCUUCUCCCUGCACAAGUACGACCUGAUCGCC |
| | | UUCCAGAAGGACGAGAAGUCCAAGGUGGAGUUCGCCUACUACAUCAACUGCGACU |
| | | CCUCCAACGGCCGGUUCUACCUGGCCUGGCACGACAAGGGCUCCAAGGAGCAGCA |
| | | GUUCCGGAUCUCCACCCAGAACCUGGUGCUGAUCCAGAAGUACCAGGUGAACGAG |
| | | CUGGGCAAGGAGAUCCGGCCCUGCCGGCUGAAGAAGCGGCCCCCCGUGCGGUCCG |
| | | GAAAGCGGACCGCCGACGGCUCCGAGUUCGAGUCCCCCAAGAAGAAGCGGAAGGU |
| | | GGAGUAGCUAGCACCAGCCUCAAGAACACCCGAAUGGAGUCUCUAAGCUACAUAA |
| | | UACCAACUUACACUUUACAAAAUGUUGUCCCCCAAAAUGUAGCCAUUCGUAUCUG |
| | | CUCCUAAUAAAAAGAAAGUUUCUUCACAUUCUCUCGAGAAAAAAAAAAAAAAAAA |
| | | AAAAAAAAAAAAAAAAAAAAAAAAAAAAAAAAAAAAAAAAAAAAAAAAAAAAAAA |
| | | AAAAAAAAAAAAAAAAAAAAAUCUAG |
| 624 | mRNA E encoding | GGGAAGCUCAGAAUAAACGCUCAACUUUGGCCGGAUCUGCCACCAUGGAGGCCUC |
| | SpyCas9 base | CCCCGCCUCCGGCCCCCGGCACCUGAUGGACCCCCACAUCUUCACCUCCAACUUC |
| | editor | AACAACGGCAUCGGCCGGCACAAGACCUACCUGUGCUACGAGGUGGAGCGGCUGG |
| | | ACAACGGCACCUCCGUGAAGAUGGACCAGCACCGGGGCUUCCUGCACAACCAGGC |
| | | CAAGAACCUGCUGUGCGGCUUCUACGGCCGGCACGCCGAGCUGCGGUUCCUGGAC |
| | | CUGGUGCCCUCCCUGCAGCUGGACCCCGCCCAGAUCUACCGGGUGACCUGGUUCA |
| | | UCUCCUGGUCCCCCUGCUUCUCCUGGGGCUGCGCCGGCGAGGUGCGGGCCUUCCU |
| | | GCAGGAGAACACCCACGUGCGGCUGCGGAUCUUCGCCGCCCGGAUCUACGACUAC |
| | | GACCCCCUGUACAAGGAGGCCCUGCAGAUGCUGCGGGACGCCGGCGCCCAGGUGU |
| | | CCAUCAUGACCUACGACGAGUUCAAGCACUGCUGGGACACCUUCGUGGACCACCA |
| | | GGGCUGCCCCUUCCAGCCCUGGGACGGCCUGGACGAGCACUCCCAGGCCCUGUCC |
| | | GGCCGGCUGCGGGCCAUCCUGCAGAACCAGGGCAACUCCGGCUCCGAGACCCCCG |
| | | GCACCUCCGAGUCCGCCACCCCCGAGUCCGACAAGAAGUACUCCAUCGGCCUGGC |
| | | CAUCGGCACCAACUCCGUGGGCUGGGCCGUGAUCACCGACGAGUACAAGGUGCCC |
| | | UCCAAGAAGUUCAAGGUGCUGGGCAACACCGACCGGCACUCCAUCAAGAAGAACC |
| | | UGAUCGGCGCCCUGCUGUUCGACUCCGGCGAGACCGCCGAGGCCACCCGGCUGAA |
| | | GCGGACCGCCCGGCGGCGGUACACCCGGCGGAAGAACCGGAUCUGCUACCUGCAG |
| | | GAGAUCUUCUCCAACGAGAUGGCCAAGGUGGACGACUCCUUCUUCCACCGGCUGG |
| | | AGGAGUCCUUCCUGGUGGAGGAGGACAAGAAGCACGAGCGGCACCCCAUCUUCGG |
| | | CAACAUCGUGGACGAGGUGGCCUACCACGAGAAGUACCCCACCAUCUACCACCUG |
| | | CGGAAGAAGCUGGUGGACUCCACCGACAAGGCCGACCUGCGGCUGAUCUACCUGG |
| | | CCCUGGCCCACAUGAUCAAGUUCCGGGGCCACUUCCUGAUCGAGGGCGACCUGAA |
| | | CCCCGACAACUCCGACGUGGACAAGCUGUUCAUCCAGCUGGUGCAGACCUACAAC |
| | | CAGCUGUUCGAGGAGAACCCCAUCAACGCCUCCGGCGUGGACGCCAAGGCCAUCC |
| | | UGUCCGCCCGGCUGUCCAAGUCCCGGCGGCUGGAGAACCUGAUCGCCCAGCUGCC |
| | | CGGCGAGAAGAAGAACGGCCUGUUCGGCAACCUGAUCGCCCUGUCCCUGGGCCUG |
| | | ACCCCCAACUUCAAGUCCAACUUCGACCUGGCCGAGGACGCCAAGCUGCAGCUGU |
| | | CCAAGGACACCUACGACGACGACCUGGACAACCUGCUGGCCCAGAUCGGCGACCA |
| | | GUACGCCGACCUGUUCCUGGCCGCCAAGAACCUGUCCGACGCCAUCCUGCUGUCC |
| | | GACAUCCUGCGGGUGAACACCGAGAUCACCAAGGCCCCCCUGUCCGCCUCCAUGA |
| | | UCAAGCGGUACGACGAGCACCACCAGGACCUGACCCUGCUGAAGGCCCUGGUGCG |
| | | GCAGCAGCUGCCCGAGAAGUACAAGGAGAUCUUCUUCGACCAGUCCAAGAACGGC |
| | | UACGCCGGCUACAUCGACGGCGGCGCCUCCCAGGAGGAGUUCUACAAGUUCAUCA |
| | | AGCCCAUCCUGGAGAAGAUGGACGGCACCGAGGAGCUGCUGGUGAAGCUGAACCG |
| | | GGAGGACCUGCUGCGGAAGCAGCGGACCUUCGACAACGGCUCCAUCCCCCACCAG |
| | | AUCCACCUGGGCGAGCUGCACGCCAUCCUGCGGCGGCAGGAGGACUUCUACCCCU |
| | | UCCUGAAGGACAACCGGGAGAAGAUCGAGAAGAUCCUGACCUUCCGGAUCCCCUA |
| | | CUACGUGGGCCCCCUGGCCCGGGGCAACUCCCGGUUCGCCUGGAUGACCCGGAAG |
| | | UCCGAGGAGACCAUCACCCCCUGGAACUUCGAGGAGGUGGUGGACAAGGGCGCCU |
| | | CCGCCCAGUCCUUCAUCGAGCGGAUGACCAACUUCGACAAGAACCUGCCCAACGA |
| | | GAAGGUGCUGCCCAAGCACUCCCUGCUGUACGAGUACUUCACCGUGUACAACGAG |
| | | CUGACCAAGGUGAAGUACGUGACCGAGGGCAUGCGGAAGCCCGCCUUCCUGUCCG |
| | | GCGAGCAGAAGAAGGCCAUCGUGGACCUGCUGUUCAAGACCAACCGGAAGGUGAC |
| | | CGUGAAGCAGCUGAAGGAGGACUACUUCAAGAAGAUCGAGUGCUUCGACUCCGUG |
| | | GAGAUCUCCGGCGUGGAGGACCGGUUCAACGCCUCCCUGGGCACCUACCACGACC |
| | | UGCUGAAGAUCAUCAAGGACAAGGACUUCCUGGACAACGAGGAGAACGAGGACAU |
| | | CCUGGAGGACAUCGUGCUGACCCUGACCCUGUUCGAGGACCGGGAGAUGAUCGAG |
| | | GAGCGGCUGAAGACCUACGCCCACCUGUUCGACGACAAGGUGAUGAAGCAGCUGA |
| | | AGCGGCGGCGGUACACCGGCUGGGGCCGGCUGUCCCGGAAGCUGAUCAACGGCAU |
| | | CCGGGACAAGCAGUCCGGCAAGACCAUCCUGGACUUCCUGAAGUCCGACGGCUUC |
| | | GCCAACCGGAACUUCAUGCAGCUGAUCCACGACGACUCCCUGACCUUCAAGGAGG |
| | | ACAUCCAGAAGGCCCAGGUGUCCGGCCAGGGCGACUCCCUGCACGAGCACAUCGC |
| | | CAACCUGGCCGGCUCCCCCGCCAUCAAGAAGGGCAUCCUGCAGACCGUGAAGGUG |
| | | GUGGACGAGCUGGUGAAGGUGAUGGGCCGGCACAAGCCCGAGAACAUCGUGAUCG |
| | | AGAUGGCCCGGGAGAACCAGACCACCCAGAAGGGCCAGAAGAACUCCCGGGAGCG |
| | | GAUGAAGCGGAUCGAGGAGGGCAUCAAGGAGCUGGGCUCCCAGAUCCUGAAGGAG |
| | | CACCCCGUGGAGAACACCCAGCUGCAGAACGAGAAGCUGUACCUGUACUACCUGC |
| | | AGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGAC |
| | | AACAAGGUGCUGACCCGGUCCGACAAGAACCGGGGCAAGUCCGACAACGUGCCCU |
| | | CCGAGGAGGUGGUGAAGAAGAUGAAGAACUACUGGCGGCAGCUGCUGAACGCCAA |
| | | GCUGAUCACCCAGCGGAAGUUCGACAACCUGACCAAGGCCGAGCGGGGCGGCCUG |
| | | UCCGAGCUGGACAAGGCCGGCUUCAUCAAGCGGCAGCUGGUGGAGACCCGGCAGA |
| | | UCACCAAGCACGUGGCCCAGAUCCUGGACUCCCGGAUGAACACCAAGUACGACGA |
| | | GAACGACAAGCUGAUCCGGGAGGUGAAGGUGAUCACCCUGAAGUCCAAGCUGGUG |
| | | UCCGACUUCCGGAAGGACUUCCAGUUCUACAAGGUGCGGGAGAUCAACAACUACC |
| | | ACCACGCCCACGACGCCUACCUGAACGCCGUGGUGGGCACCGCCCUGAUCAAGAA |
| | | GUACCCCAAGCUGGAGUCCGAGUUCGUGUACGGCGACUACAAGGUGUACGACGUG |
| | | CGGAAGAUGAUCGCCAAGUCCGAGCAGGAGAUCGGCAAGGCCACCGCCAAGUACU |
| | | UCUUCUACUCCAACAUCAUGAACUUCUUCAAGACCGAGAUCACCCUGGCCAACGG |
| | | CGAGAUCCGGAAGCGGCCCCUGAUCGAGACCAACGGCGAGACCGGCGAGAUCGUG |
| | | UGGGACAAGGGCCGGGACUUCGCCACCGUGCGGAAGGUGCUGUCCAUGCCCCAGG |
| | | UGAACAUCGUGAAGAAGACCGAGGUGCAGACCGGCGGCUUCUCCAAGGAGUCCAU |
| | | CCUGCCCAAGCGGAACUCCGACAAGCUGAUCGCCCGGAAGAAGGACUGGGACCCC |
| | | AAGAAGUACGGCGGCUUCGACUCCCCCACCGUGGCCUACUCCGUGCUGGUGGUGG |
| | | CCAAGGUGGAGAAGGGCAAGUCCAAGAAGCUGAAGUCCGUGAAGGAGCUGCUGGG |
| | | CAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACU |
| | | CCCUGUUCGAGCUGGAGAACGGCCGGAAGCGGAUGCUGGCCUCCGCCGGCGAGCU |
| | | GCAGAAGGGCAACGAGCUGGCCCUGCCCUCCAAGUACGUGAACUUCCUGUACCUG |
| | | GCCUCCCACUACGAGAAGCUGAAGGGCUCCCCCGAGGACAACGAGCAGAAGCAGC |
| | | UGUUCGUGGAGCAGCACAAGCACUACCUGGACGAGAUCAUCGAGCAGAUCUCCGA |
| | | GUUCUCCAAGCGGGUGAUCCUGGCCGACGCCAACCUGGACAAGGUGCUGUCCGCC |
| | | UACAACAAGCACCGGGACAAGCCCAUCCGGGAGCAGGCCGAGAACAUCAUCCACC |
| | | UGUUCACCCUGACCAACCUGGGCGCCCCCGCCGCCUUCAAGUACUUCGACACCAC |
| | | CAUCGACCGGAAGCGGUACACCUCCACCAAGGAGGUGCUGGACGCCACCCUGAUC |
| | | CACCAGUCCAUCACCGGCCUGUACGAGACCCGGAUCGACCUGUCCCAGCUGGGCG |
| | | GCGACGGCGGCGGCUCCCCCAAGAAGAAGCGGAAGGUGUGACUAGCACCAGCCUC |
| | | AAGAACACCCGAAUGGAGUCUCUAAGCUACAUAAUACCAACUUACACUUUACAAA |
| | | AUGUUGUCCCCCAAAAUGUAGCCAUUCGUAUCUGCUCCUAAUAAAAAGAAAGUUU |
| | | CUUCACAUUCUCUCGAGAAAAAAAAAAAAUGGAAAAAAAAAAAACGGAAAAAAAA |
| | | AAAAGGUAAAAAAAAAAAAUAUAAAAAAAAAAAACAUAAAAAAAAAAAACGAAAA |
| | | AAAAAAAACGUAAAAAAAAAAAACUCAAAAAAAAAAAAGAUAAAAAAAAAAAACC |
| | | UAAAAAAAAAAAAUGUAAAAAAAAAAAAGGGAAAAAAAAAAAACGCAAAAAAAAA |
| | | AAACACAAAAAAAAAAAAUGCAAAAAAAAAAAAUCGAAAAAAAAAAAAUCUAAAA |
| | | AAAAAAAACGAAAAAAAAAAAACCCAAAAAAAAAAAAGACAAAAAAAAAAAAUAG |
| | | AAAAAAAAAAAAGUUAAAAAAAAAAAACUGAAAAAAAAAAAAUUUAAAAAAAAAA |
| | | AAUCUAG |
| 625 | mRNA G encoding | GGGAAGCUCAGAAUAAACGCUCAACUUUGGCCGGAUCUGCCACCAUGACCAACCU |
| | UGI | GUCCGACAUCAUCGAGAAGGAGACCGGCAAGCAGCUGGUGAUCCAGGAGUCCAUC |
| | | CUGAUGCUGCCCGAGGAGGUGGAGGAGGUGAUCGGCAACAAGCCCGAGUCCGACA |
| | | UCCUGGUGCACACCGCCUACGACGAGUCCACCGACGAGAACGUGAUGCUGCUGAC |
| | | CUCCGACGCCCCCGAGUACAAGCCCUGGGCCCUGGUGAUCCAGGACUCCAACGGC |
| | | GAGAACAAGAUCAAGAUGCUGUCCGGCGGCUCCAAGCGGACCGCCGACGGCUCCG |
| | | AGUUCGAGUCCCCCAAGAAGAAGCGGAAGGUGGAGUGAUAGCUAGCACCAGCCUC |
| | | AAGAACACCCGAAUGGAGUCUCUAAGCUACAUAAUACCAACUUACACUUUACAAA |
| | | AUGUUGUCCCCCAAAAUGUAGCCAUUCGUAUCUGCUCCUAAUAAAAAGAAAGUUU |
| | | CUUCACAUUCUCUCGAGAAAAAAAAAAAAUGGAAAAAAAAAAAACGGAAAAAAAA |
| | | AAAAGGUAAAAAAAAAAAAUAUAAAAAAAAAAAACAUAAAAAAAAAAAACGAAAA |
| | | AAAAAAAACGUAAAAAAAAAAAACUCAAAAAAAAAAAAGAUAAAAAAAAAAAACC |
| | | UAAAAAAAAAAAAUGUAAAAAAAAAAAAGGGAAAAAAAAAAAACGCAAAAAAAAA |
| | | AAACACAAAAAAAAAAAAUGCAAAAAAAAAAAAUCGAAAAAAAAAAAAUCUAAAA |
| | | AAAAAAAACGAAAAAAAAAAAACCCAAAAAAAAAAAAGACAAAAAAAAAAAAUAG |
| | | AAAAAAAAAAAAGUUAAAAAAAAAAAACUGAAAAAAAAAAAAUUUAAAAAAAAAA |
| | | AAUCUAG |
| 626 | mRNA H encoding | GGGaagctcagaataaacgctcaactttggccggatctgccacCATGGCCGCCTT |
| | Nme2Cas 9 | CAAGCCCAACCCCATCAACTACATCCTGGGCCTGGACATCGGCATCGCCTCCGTG |
| | | GGCTGGGCCATGGTGGAGATCGACGAGGAGGAGAACCCCATCCGGCTGATCGACC |
| | | TGGGCGTGCGGGTGTTCGAGCGGGCCGAGGTGCCCAAGACCGGCGACTCCCTGGC |
| | | CATGGCCCGGCGGCTGGCCCGGTCCGTGCGGCGGCTGACCCGGCGGCGGGCCCAC |
| | | CGGCTGCTGCGGGCCCGGCGGCTGCTGAAGCGGGAGGGCGTGCTGCAGGCCGCCG |
| | | ACTTCGACGAGAACGGCCTGATCAAGTCCCTGCCCAACACCCCCTGGCAGCTGCG |
| | | GGCCGCCGCCCTGGACCGGAAGCTGACCCCCCTGGAGTGGTCCGCCGTGCTGCTG |
| | | CACCTGATCAAGCACCGGGGCTACCTGTCCCAGCGGAAGAACGAGGGCGAGACCG |
| | | CCGACAAGGAGCTGGGCGCCCTGCTGAAGGGCGTGGCCAACAACGCCCACGCCCT |
| | | GCAGACCGGCGACTTCCGGACCCCCGCCGAGCTGGCCCTGAACAAGTTCGAGAAG |
| | | GAGTCCGGCCACATCCGGAACCAGCGGGGCGACTACTCCCACACCTTCTCCCGGA |
| | | AGGACCTGCAGGCCGAGCTGATCCTGCTGTTCGAGAAGCAGAAGGAGTTCGGCAA |
| | | CCCCCACGTGTCCGGCGGCCTGAAGGAGGGCATCGAGACCCTGCTGATGACCCAG |
| | | CGGCCCGCCCTGTCCGGCGACGCCGTGCAGAAGATGCTGGGCCACTGCACCTTCG |
| | | AGCCCGCCGAGCCCAAGGCCGCCAAGAACACCTACACCGCCGAGCGGTTCATCTG |
| | | GCTGACCAAGCTGAACAACCTGCGGATCCTGGAGCAGGGCTCCGAGCGGCCCCTG |
| | | ACCGACACCGAGCGGGCCACCCTGATGGACGAGCCCTACCGGAAGTCCAAGCTGA |
| | | CCTACGCCCAGGCCCGGAAGCTGCTGGGCCTGGAGGACACCGCCTTCTTCAAGGG |
| | | CCTGCGGTACGGCAAGGACAACGCCGAGGCCTCCACCCTGATGGAGATGAAGGCC |
| | | TACCACGCCATCTCCCGGGCCCTGGAGAAGGAGGGCCTGAAGGACAAGAAGTCCC |
| | | CCCTGAACCTGTCCTCCGAGCTGCAGGACGAGATCGGCACCGCCTTCTCCCTGTT |
| | | CAAGACCGACGAGGACATCACCGGCCGGCTGAAGGACCGGGTGCAGCCCGAGATC |
| | | CTGGAGGCCCTGCTGAAGCACATCTCCTTCGACAAGTTCGTGCAGATCTCCCTGA |
| | | AGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATC |
| | | TACCTGCCCCCCATCCCCGCCGACGAGATCCGGAACCCCGTGGTGCTGCGGGCCC |
| | | TGTCCCAGGCCCGGAAGGTGATCAACGGCGTGGTGCGGCGGTACGGCTCCCCCGC |
| | | CCGGATCCACATCGAGACCGCCCGGGAGGTGGGCAAGTCCTTCAAGGACCGGAAG |
| | | GAGATCGAGAAGCGGCAGGAGGAGAACCGGAAGGACCGGGAGAAGGCCGCCGCCA |
| | | AGTTCCGGGAGTACTTCCCCAACTTCGTGGGCGAGCCCAAGTCCAAGGACATCCT |
| | | GAAGCTGCGGCTGTACGAGCAGCAGCACGGCAAGTGCCTGTACTCCGGCAAGGAG |
| | | ATCAACCTGGTGCGGCTGAACGAGAAGGGCTACGTGGAGATCGACCACGCCCTGC |
| | | CCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGAC |
| | | AACTCCCGGGAGTGGCAGGAGTTCAAGGCCCGGGTGGAGACCTCCCGGTTCCCCC |
| | | GGTCCAAGAAGCAGCGGATCCTGCTGCAGAAGTTCGACGAGGACGGCTTCAAGGA |
| | | GTGCAACCTGAACGACACCCGGTACGTGAACCGGTTCCTGTGCCAGTTCGTGGCC |
| | | GACCACATCCTGCTGACCGGCAAGGGCAAGCGGCGGGTGTTCGCCTCCAACGGCC |
| | | AGATCACCAACCTGCTGCGGGGCTTCTGGGGCCTGCGGAAGGTGCGGGCCGAGAA |
| | | CGACCGGCACCACGCCCTGGACGCCGTGGTGGTGGCCTGCTCCACCGTGGCCATG |
| | | CAGCAGAAGATCACCCGGTTCGTGCGGTACAAGGAGATGAACGCCTTCGACGGCA |
| | | AGACCATCGACAAGGAGACCGGCAAGGTGCTGCACCAGAAGACCCACTTCCCCCA |
| | | GCCCTGGGAGTTCTTCGCCCAGGAGGTGATGATCCGGGTGTTCGGCAAGCCCGAC |
| | | GGCAAGCCCGAGTTCGAGGAGGCCGACACCCCCGAGAAGCTGCGGACCCTGCTGG |
| | | CCGAGAAGCTGTCCTCCCGGCCCGAGGCCGTGCACGAGTACGTGACCCCCCTGTT |
| | | CGTGTCCCGGGCCCCCAACCGGAAGATGTCCGGCGCCCACAAGGACACCCTGCGG |
| | | TCCGCCAAGCGGTTCGTGAAGCACAACGAGAAGATCTCCGTGAAGCGGGTGTGGC |
| | | TGACCGAGATCAAGCTGGCCGACCTGGAGAACATGGTGAACTACAAGAACGGCCG |
| | | GGAGATCGAGCTGTACGAGGCCCTGAAGGCCCGGCTGGAGGCCTACGGCGGCAAC |
| | | GCCAAGCAGGCCTTCGACCCCAAGGACAACCCCTTCTACAAGAAGGGCGGCCAGC |
| | | TGGTGAAGGCCGTGCGGGTGGAGAAGACCCAGGAGTCCGGCGTGCTGCTGAACAA |
| | | GAAGAACGCCTACACCATCGCCGACAACGGCGACATGGTGCGGGTGGACGTGTTC |
| | | TGCAAGGTGGACAAGTCCGGCGGCGGCTCCCCCAAGAAGAAGCGGAAGGTGTCCG |
| | | GCGGCTCCGGCAAGAACCAGTACTTCATCGTGCCCATCTACGCCTGGCAGGTGGC |
| | | CGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGA |
| | | AGTCCAAGGTGGAGTTCGCCTACTACATCAACTGCGACTCCTCCAACGGCCGGTT |
| | | CTACCTGGCCTGGCACGACAAGGGCTCCAAGGAGCAGCAGTTCCGGATCTCCACC |
| | | CAGAACCTGGTGCTGATCCAGAAGTACCAGGTGAACGAGCTGGGCAAGGAGATCC |
| | | GGCCCTGCCGGCTGAAGAAGCGGCCCCCCGTGCGGTAGCTAGCaccagcctcaag |
| | | aacacccgaatggagtctctaagctacataataccaacttacactttacaaaatg |
| | | ttgtcccccaaaatgtagccattcgtatctgctcctaataaaaagaaagtttctt |
| | | cacattctCTCGAGAAAAAAAAAAAATGGAAAAAAAAAAAACGGAAAAAAAAAAA |
| | | AGGTAAAAAAAAAAAATATAAAAAAAAAAAACATAAAAAAAAAAAACGAAAAAAA |
| | | AAAAACGTAAAAAAAAAAAACTCAAAAAAAAAAAAGATAAAAAAAAAAAACCTAA |
| | | AAAAAAAAAATGTAAAAAAAAAAAAGGGAAAAAAAAAAAACGCAAAAAAAAAAAA |
| | | CACAAAAAAAAAAAATGCAAAAAAAAAAAATCGAAAAAAAAAAAATCTAAAAAAA |
| | | AAAAACGAAAAAAAAAAAACCCAAAAAAAAAAAAGACAAAAAAAAAAAATAGAAA |
| | | AAAAAAAAAGTTAAAAAAAAAAAACTGAAAAAAAAAAAATTTAAAAAAAAAAAAT |
| 627 | mRNA I encoding | GGGaagctcagaataaacgctcaactttggccggatctgccacCATGGTGCCCAA |
| | Nme2Cas 9 | GAAGAAGCGGAAGGTGGCCGCCTTCAAGCCCAACCCCATCAACTACATCCTGGGC |
| | | CTGGACATCGGCATCGCCTCCGTGGGCTGGGCCATGGTGGAGATCGACGAGGAGG |
| | | AGAACCCCATCCGGCTGATCGACCTGGGCGTGCGGGTGTTCGAGCGGGCCGAGGT |
| | | GCCCAAGACCGGCGACTCCCTGGCCATGGCCCGGCGGCTGGCCCGGTCCGTGCGG |
| | | CGGCTGACCCGGCGGCGGGCCCACCGGCTGCTGCGGGCCCGGCGGCTGCTGAAGC |
| | | GGGAGGGCGTGCTGCAGGCCGCCGACTTCGACGAGAACGGCCTGATCAAGTCCCT |
| | | GCCCAACACCCCCTGGCAGCTGCGGGCCGCCGCCCTGGACCGGAAGCTGACCCCC |
| | | CTGGAGTGGTCCGCCGTGCTGCTGCACCTGATCAAGCACCGGGGCTACCTGTCCC |
| | | AGCGGAAGAACGAGGGCGAGACCGCCGACAAGGAGCTGGGCGCCCTGCTGAAGGG |
| | | CGTGGCCAACAACGCCCACGCCCTGCAGACCGGCGACTTCCGGACCCCCGCCGAG |
| | | CTGGCCCTGAACAAGTTCGAGAAGGAGTCCGGCCACATCCGGAACCAGCGGGGCG |
| | | ACTACTCCCACACCTTCTCCCGGAAGGACCTGCAGGCCGAGCTGATCCTGCTGTT |
| | | CGAGAAGCAGAAGGAGTTCGGCAACCCCCACGTGTCCGGCGGCCTGAAGGAGGGC |
| | | ATCGAGACCCTGCTGATGACCCAGCGGCCCGCCCTGTCCGGCGACGCCGTGCAGA |
| | | AGATGCTGGGCCACTGCACCTTCGAGCCCGCCGAGCCCAAGGCCGCCAAGAACAC |
| | | CTACACCGCCGAGCGGTTCATCTGGCTGACCAAGCTGAACAACCTGCGGATCCTG |
| | | GAGCAGGGCTCCGAGCGGCCCCTGACCGACACCGAGCGGGCCACCCTGATGGACG |
| | | AGCCCTACCGGAAGTCCAAGCTGACCTACGCCCAGGCCCGGAAGCTGCTGGGCCT |
| | | GGAGGACACCGCCTTCTTCAAGGGCCTGCGGTACGGCAAGGACAACGCCGAGGCC |
| | | TCCACCCTGATGGAGATGAAGGCCTACCACGCCATCTCCCGGGCCCTGGAGAAGG |
| | | AGGGCCTGAAGGACAAGAAGTCCCCCCTGAACCTGTCCTCCGAGCTGCAGGACGA |
| | | GATCGGCACCGCCTTCTCCCTGTTCAAGACCGACGAGGACATCACCGGCCGGCTG |
| | | AAGGACCGGGTGCAGCCCGAGATCCTGGAGGCCCTGCTGAAGCACATCTCCTTCG |
| | | ACAAGTTCGTGCAGATCTCCCTGAAGGCCCTGCGGCGGATCGTGCCCCTGATGGA |
| | | GCAGGGCAAGCGGTACGACGAGGCCTGCGCCGAGATCTACGGCGACCACTACGGC |
| | | AAGAAGAACACCGAGGAGAAGATCTACCTGCCCCCCATCCCCGCCGACGAGATCC |
| | | GGAACCCCGTGGTGCTGCGGGCCCTGTCCCAGGCCCGGAAGGTGATCAACGGCGT |
| | | GGTGCGGCGGTACGGCTCCCCCGCCCGGATCCACATCGAGACCGCCCGGGAGGTG |
| | | GGCAAGTCCTTCAAGGACCGGAAGGAGATCGAGAAGCGGCAGGAGGAGAACCGGA |
| | | AGGACCGGGAGAAGGCCGCCGCCAAGTTCCGGGAGTACTTCCCCAACTTCGTGGG |
| | | CGAGCCCAAGTCCAAGGACATCCTGAAGCTGCGGCTGTACGAGCAGCAGCACGGC |
| | | AAGTGCCTGTACTCCGGCAAGGAGATCAACCTGGTGCGGCTGAACGAGAAGGGCT |
| | | ACGTGGAGATCGACCACGCCCTGCCCTTCTCCCGGACCTGGGACGACTCCTTCAA |
| | | CAACAAGGTGCTGGTGCTGGGCTCCGAGAACCAGAACAAGGGCAACCAGACCCCC |
| | | TACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAA |
| | | GTTCGACGAGGACGGCTTCAAGGAGTGCAACCTGAACGACACCCGGTACGTGAAC |
| | | CGGTTCCTGTGCCAGTTCGTGGCCGACCACATCCTGCTGACCGGCAAGGGCAAGC |
| | | GGCGGGTGTTCGCCTCCAACGGCCAGATCACCAACCTGCTGCGGGGCTTCTGGGG |
| | | CCTGCGGAAGGTGCGGGCCGAGAACGACCGGCACCACGCCCTGGACGCCGTGGTG |
| | | GTGGCCTGCTCCACCGTGGCCATGCAGCAGAAGATCACCCGGTTCGTGCGGTACA |
| | | AGGAGATGAACGCCTTCGACGGCAAGACCATCGACAAGGAGACCGGCAAGGTGCT |
| | | GCACCAGAAGACCCACTTCCCCCAGCCCTGGGAGTTCTTCGCCCAGGAGGTGATG |
| | | ATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGT |
| | | GCACGAGTACGTGACCCCCCTGTTCGTGTCCCGGGCCCCCAACCGGAAGATGTCC |
| | | GGCGCCCACAAGGACACCCTGCGGTCCGCCAAGCGGTTCGTGAAGCACAACGAGA |
| | | AGATCTCCGTGAAGCGGGTGTGGCTGACCGAGATCAAGCTGGCCGACCTGGAGAA |
| | | CATGGTGAACTACAAGAACGGCCGGGAGATCGAGCTGTACGAGGCCCTGAAGGCC |
| | | CGGCTGGAGGCCTACGGCGGCAACGCCAAGCAGGCCTTCGACCCCAAGGACAACC |
| | | CCTTCTACAAGAAGGGCGGCCAGCTGGTGAAGGCCGTGCGGGTGGAGAAGACCCA |
| | | GGAGTCCGGCGTGCTGCTGAACAAGAAGAACGCCTACACCATCGCCGACAACGGC |
| | | GACATGGTGCGGGTGGACGTGTTCTGCAAGGTGGACAAGAAGGGCAAGAACCAGT |
| | | ACTTCATCGTGCCCATCTACGCCTGGCAGGTGGCCGAGAACATCCTGCCCGACAT |
| | | CGACTGCAAGGGCTACCGGATCGACGACTCCTACACCTTCTGCTTCTCCCTGCAC |
| | | AAGTACGACCTGATCGCCTTCCAGAAGGACGAGAAGTCCAAGGTGGAGTTCGCCT |
| | | ACTACATCAACTGCGACTCCTCCAACGGCCGGTTCTACCTGGCCTGGCACGACAA |
| | | GGGCTCCAAGGAGCAGCAGTTCCGGATCTCCACCCAGAACCTGGTGCTGATCCAG |
| | | AAGTACCAGGTGAACGAGCTGGGCAAGGAGATCCGGCCCTGCCGGCTGAAGAAGC |
| | | GGCCCCCCGTGCGGTACCCCTACGACGTGCCCGACTACGCCGCCGCCCCCGCCGC |
| | | CAAGAAGAAGAAGCTGGACTAGCTAGCaccagcctcaagaacacccgaatggagt |
| | | ctctaagctacataataccaacttacactttacaaaatgttgtcccccaaaatgt |
| | | agccattcgtatctgctcctaataaaaagaaagtttcttcacattctCTCGAGAA |
| | | AAAAAAAAAATGGAAAAAAAAAAAACGGAAAAAAAAAAAAGGTAAAAAAAAAAAA |
| | | TATAAAAAAAAAAAACATAAAAAAAAAAAACGAAAAAAAAAAAACGTAAAAAAAA |
| | | AAAACTCAAAAAAAAAAAAGATAAAAAAAAAAAACCTAAAAAAAAAAAATGTAAA |
| | | AAAAAAAAAGGGAAAAAAAAAAAACGCAAAAAAAAAAAACACAAAAAAAAAAAAT |
| | | GCAAAAAAAAAAAATCGAAAAAAAAAAAATCTAAAAAAAAAAAACGAAAAAAAAA |
| | | AAACCCAAAAAAAAAAAAGACAAAAAAAAAAAATAGAAAAAAAAAAAAGTTAAAA |
| | | AAAAAAAACTGAAAAAAAAAAAATTTAAAAAAAAAAAAT |
| 628 | mRNA J encoding | GGGAAGCUCAGAAUAAACGCUCAACUUUGGCCGGAUCUGCCACCAUGGUGCCCAA |
| | Nme2Cas 9 | GAAGAAGCGGAAGGUGGAGGACAAGCGGCCCGCCGCCACCAAGAAGGCCGGCCAG |
| | | GCCAAGAAGAAGAAGAUGGCCGCCUUCAAGCCCAACCCCAUCAACUACAUCCUGG |
| | | GCCUGGACAUCGGCAUCGCCUCCGUGGGCUGGGCCAUGGUGGAGAUCGACGAGGA |
| | | GGAGAACCCCAUCCGGCUGAUCGACCUGGGCGUGCGGGUGUUCGAGCGGGCCGAG |
| | | GUGCCCAAGACCGGCGACUCCCUGGCCAUGGCCCGGCGGCUGGCCCGGUCCGUGC |
| | | GGCGGCUGACCCGGCGGCGGGCCCACCGGCUGCUGCGGGCCCGGCGGCUGCUGAA |
| | | GCGGGAGGGCGUGCUGCAGGCCGCCGACUUCGACGAGAACGGCCUGAUCAAGUCC |
| | | CUGCCCAACACCCCCUGGCAGCUGCGGGCCGCCGCCCUGGACCGGAAGCUGACCC |
| | | CCCUGGAGUGGUCCGCCGUGCUGCUGCACCUGAUCAAGCACCGGGGCUACCUGUC |
| | | CCAGCGGAAGAACGAGGGCGAGACCGCCGACAAGGAGCUGGGCGCCCUGCUGAAG |
| | | GGCGUGGCCAACAACGCCCACGCCCUGCAGACCGGCGACUUCCGGACCCCCGCCG |
| | | AGCUGGCCCUGAACAAGUUCGAGAAGGAGUCCGGCCACAUCCGGAACCAGCGGGG |
| | | CGACUACUCCCACACCUUCUCCCGGAAGGACCUGCAGGCCGAGCUGAUCCUGCUG |
| | | UUCGAGAAGCAGAAGGAGUUCGGCAACCCCCACGUGUCCGGCGGCCUGAAGGAGG |
| | | GCAUCGAGACCCUGCUGAUGACCCAGCGGCCCGCCCUGUCCGGCGACGCCGUGCA |
| | | GAAGAUGCUGGGCCACUGCACCUUCGAGCCCGCCGAGCCCAAGGCCGCCAAGAAC |
| | | ACCUACACCGCCGAGCGGUUCAUCUGGCUGACCAAGCUGAACAACCUGCGGAUCC |
| | | UGGAGCAGGGCUCCGAGCGGCCCCUGACCGACACCGAGCGGGCCACCCUGAUGGA |
| | | CGAGCCCUACCGGAAGUCCAAGCUGACCUACGCCCAGGCCCGGAAGCUGCUGGGC |
| | | CUGGAGGACACCGCCUUCUUCAAGGGCCUGCGGUACGGCAAGGACAACGCCGAGG |
| | | CCUCCACCCUGAUGGAGAUGAAGGCCUACCACGCCAUCUCCCGGGCCCUGGAGAA |
| | | GGAGGGCCUGAAGGACAAGAAGUCCCCCCUGAACCUGUCCUCCGAGCUGCAGGAC |
| | | GAGAUCGGCACCGCCUUCUCCCUGUUCAAGACCGACGAGGACAUCACCGGCCGGC |
| | | UGAAGGACCGGGUGCAGCCCGAGAUCCUGGAGGCCCUGCUGAAGCACAUCUCCUU |
| | | CGACAAGUUCGUGCAGAUCUCCCUGAAGGCCCUGCGGCGGAUCGUGCCCCUGAUG |
| | | GAGCAGGGCAAGCGGUACGACGAGGCCUGCGCCGAGAUCUACGGCGACCACUACG |
| | | GCAAGAAGAACACCGAGGAGAAGAUCUACCUGCCCCCCAUCCCCGCCGACGAGAU |
| | | CCGGAACCCCGUGGUGCUGCGGGCCCUGUCCCAGGCCCGGAAGGUGAUCAACGGC |
| | | GUGGUGCGGCGGUACGGCUCCCCCGCCCGGAUCCACAUCGAGACCGCCCGGGAGG |
| | | UGGGCAAGUCCUUCAAGGACCGGAAGGAGAUCGAGAAGCGGCAGGAGGAGAACCG |
| | | GAAGGACCGGGAGAAGGCCGCCGCCAAGUUCCGGGAGUACUUCCCCAACUUCGUG |
| | | GGCGAGCCCAAGUCCAAGGACAUCCUGAAGCUGCGGCUGUACGAGCAGCAGCACG |
| | | GCAAGUGCCUGUACUCCGGCAAGGAGAUCAACCUGGUGCGGCUGAACGAGAAGGG |
| | | CUACGUGGAGAUCGACCACGCCCUGCCCUUCUCCCGGACCUGGGACGACUCCUUC |
| | | AACAACAAGGUGCUGGUGCUGGGCUCCGAGAACCAGAACAAGGGCAACCAGACCC |
| | | CCUACGAGUACUUCAACGGCAAGGACAACUCCCGGGAGUGGCAGGAGUUCAAGGC |
| | | CCGGGUGGAGACCUCCCGGUUCCCCCGGUCCAAGAAGCAGCGGAUCCUGCUGCAG |
| | | AAGUUCGACGAGGACGGCUUCAAGGAGUGCAACCUGAACGACACCCGGUACGUGA |
| | | ACCGGUUCCUGUGCCAGUUCGUGGCCGACCACAUCCUGCUGACCGGCAAGGGCAA |
| | | GCGGCGGGUGUUCGCCUCCAACGGCCAGAUCACCAACCUGCUGCGGGGCUUCUGG |
| | | GGCCUGCGGAAGGUGCGGGCCGAGAACGACCGGCACCACGCCCUGGACGCCGUGG |
| | | UGGUGGCCUGCUCCACCGUGGCCAUGCAGCAGAAGAUCACCCGGUUCGUGCGGUA |
| | | CAAGGAGAUGAACGCCUUCGACGGCAAGACCAUCGACAAGGAGACCGGCAAGGUG |
| | | CUGCACCAGAAGACCCACUUCCCCCAGCCCUGGGAGUUCUUCGCCCAGGAGGUGA |
| | | UGAUCCGGGUGUUCGGCAAGCCCGACGGCAAGCCCGAGUUCGAGGAGGCCGACAC |
| | | CCCCGAGAAGCUGCGGACCCUGCUGGCCGAGAAGCUGUCCUCCCGGCCCGAGGCC |
| | | GUGCACGAGUACGUGACCCCCCUGUUCGUGUCCCGGGCCCCCAACCGGAAGAUGU |
| | | CCGGCGCCCACAAGGACACCCUGCGGUCCGCCAAGCGGUUCGUGAAGCACAACGA |
| | | GAAGAUCUCCGUGAAGCGGGUGUGGCUGACCGAGAUCAAGCUGGCCGACCUGGAG |
| | | AACAUGGUGAACUACAAGAACGGCCGGGAGAUCGAGCUGUACGAGGCCCUGAAGG |
| | | CCCGGCUGGAGGCCUACGGCGGCAACGCCAAGCAGGCCUUCGACCCCAAGGACAA |
| | | CCCCUUCUACAAGAAGGGCGGCCAGCUGGUGAAGGCCGUGCGGGUGGAGAAGACC |
| | | CAGGAGUCCGGCGUGCUGCUGAACAAGAAGAACGCCUACACCAUCGCCGACAACG |
| | | GCGACAUGGUGCGGGUGGACGUGUUCUGCAAGGUGGACAAGAAGGGCAAGAACCA |
| | | GUACUUCAUCGUGCCCAUCUACGCCUGGCAGGUGGCCGAGAACAUCCUGCCCGAC |
| | | AUCGACUGCAAGGGCUACCGGAUCGACGACUCCUACACCUUCUGCUUCUCCCUGC |
| | | ACAAGUACGACCUGAUCGCCUUCCAGAAGGACGAGAAGUCCAAGGUGGAGUUCGC |
| | | CUACUACAUCAACUGCGACUCCUCCAACGGCCGGUUCUACCUGGCCUGGCACGAC |
| | | AAGGGCUCCAAGGAGCAGCAGUUCCGGAUCUCCACCCAGAACCUGGUGCUGAUCC |
| | | AGAAGUACCAGGUGAACGAGCUGGGCAAGGAGAUCCGGCCCUGCCGGCUGAAGAA |
| | | GCGGCCCCCCGUGCGGGAGGACAAGCGGCCCGCCGCCACCAAGAAGGCCGGCCAG |
| | | GCCAAGAAGAAGAAGUACCCCUACGACGUGCCCGACUACGCCGGCUACCCCUACG |
| | | ACGUGCCCGACUACGCCGGCUCCUACCCCUACGACGUGCCCGACUACGCCGCCGC |
| | | CCCCGCCGCCAAGAAGAAGAAGCUGGACUAGCUAGCACCAGCCUCAAGAACACCC |
| | | GAAUGGAGUCUCUAAGCUACAUAAUACCAACUUACACUUUACAAAAUGUUGUCCC |
| | | CCAAAAUGUAGCCAUUCGUAUCUGCUCCUAAUAAAAAGAAAGUUUCUUCACAUUC |
| | | UCUCGAGAAAAAAAAAAAAUGGAAAAAAAAAAAACGGAAAAAAAAAAAAGGUAAA |
| | | AAAAAAAAAUAUAAAAAAAAAAAACAUAAAAAAAAAAAACGAAAAAAAAAAAACG |
| | | UAAAAAAAAAAAACUCAAAAAAAAAAAAGAUAAAAAAAAAAAACCUAAAAAAAAA |
| | | AAAUGUAAAAAAAAAAAAGGGAAAAAAAAAAAACGCAAAAAAAAAAAACACAAAA |
| | | AAAAAAAAUGCAAAAAAAAAAAAUCGAAAAAAAAAAAAUCUAAAAAAAAAAAACG |
| | | AAAAAAAAAAAACCCAAAAAAAAAAAAGACAAAAAAAAAAAAUAGAAAAAAAAAA |
| | | AAGUUAAAAAAAAAAAACUGAAAAAAAAAAAAUUUAAAAAAAAAAAAUCUAG |
| 629 | mRNA K encoding | GGGaagctcagaataaacgctcaactttggccggatctgccacCatggccgcctt |
| | Nme2Cas 9 | caagcccaaccccatcaactacatcctgggcctggacatcggcatcgcctccgtg |
| | | ggctgggccatggtggagatcgacgaggaggagaaccccatccggctgatcgacc |
| | | tgggcgtgcgggtgttcgagcgggccgaggtgcccaagaccggcgactccctggc |
| | | catggcccggcggctggcccggtccgtgcggcggctgacccggcggcgggcccac |
| | | cggctgctgcgggcccggcggctgctgaagcgggagggcgtgctgcaggccgccg |
| | | acttcgacgagaacggcctgatcaagtccctgcccaacaccccctggcagctgcg |
| | | ggccgccgccctggaccggaagctgacccccctggagtggtccgccgtgctgctg |
| | | cacctgatcaagcaccggggctacctgtcccagcggaagaacgagggcgagaccg |
| | | ccgacaaggagctgggcgccctgctgaagggcgtggccaacaacgcccacgccct |
| | | gcagaccggcgacttccggacccccgccgagctggccctgaacaagttcgagaag |
| | | gagtccggccacatccggaaccagcggggcgactactcccacaccttctcccgga |
| | | aggacctgcaggccgagctgatcctgctgttcgagaagcagaaggagttcggcaa |
| | | cccccacgtgtccggcggcctgaaggagggcatcgagaccctgctgatgacccag |
| | | cggcccgccctgtccggcgacgccgtgcagaagatgctgggccactgcaccttcg |
| | | agcccgccgagcccaaggccgccaagaacacctacaccgccgagcggttcatctg |
| | | gctgaccaagctgaacaacctgcggatcctggagcagggctccgagcggcccctg |
| | | accgacaccgagcgggccaccctgatggacgagccctaccggaagtccaagctga |
| | | cctacgcccaggcccggaagctgctgggcctggaggacaccgccttcttcaaggg |
| | | cctgcggtacggcaaggacaacgccgaggcctccaccctgatggagatgaaggcc |
| | | taccacgccatctcccgggccctggagaaggagggcctgaaggacaagaagtccc |
| | | ccctgaacctgtcctccgagctgcaggacgagatcggcaccgccttctccctgtt |
| | | caagaccgacgaggacatcaccggccggctgaaggaccgggtgcagcccgagatc |
| | | ctggaggccctgctgaagcacatctccttcgacaagttcgtgcagatctccctga |
| | | aggccctgcggcggatcgtgcccctgatggagcagggcaagcggtacgacgaggc |
| | | ctgcgccgagatctacggcgaccactacggcaagaagaacaccgaggagaagatc |
| | | tacctgccccccatccccgccgacgagatccggaaccccgtggtgctgcgggccc |
| | | tgtcccaggcccggaaggtgatcaacggcgtggtgcggcggtacggctcccccgc |
| | | ccggatccacatcgagaccgcccgggaggtgggcaagtccttcaaggaccggaag |
| | | gagatcgagaagcggcaggaggagaaccggaaggaccgggagaaggccgccgcca |
| | | agttccgggagtacttccccaacttcgtgggcgagcccaagtccaaggacatcct |
| | | gaagctgcggctgtacgagcagcagcacggcaagtgcctgtactccggcaaggag |
| | | atcaacctggtgcggctgaacgagaagggctacgtggagatcgaccacgccctgc |
| | | ccttctcccggacctgggacgactccttcaacaacaaggtgctggtgctgggctc |
| | | cgagaaccagaacaagggcaaccagaccccctacgagtacttcaacggcaaggac |
| | | aactcccgggagtggcaggagttcaaggcccgggtggagacctcccggttccccc |
| | | ggtccaagaagcagcggatcctgctgcagaagttcgacgaggacggcttcaagga |
| | | gtgcaacctgaacgacacccggtacgtgaaccggttcctgtgccagttcgtggcc |
| | | gaccacatcctgctgaccggcaagggcaagcggcgggtgttcgcctccaacggcc |
| | | agatcaccaacctgctgcggggcttctggggcctgcggaaggtgcgggccgagaa |
| | | cgaccggcaccacgccctggacgccgtggtggtggcctgctccaccgtggccatg |
| | | cagcagaagatcacccggttcgtgcggtacaaggagatgaacgccttcgacggca |
| | | agaccatcgacaaggagaccggcaaggtgctgcaccagaagacccacttccccca |
| | | gccctgggagttcttcgcccaggaggtgatgatccgggtgttcggcaagcccgac |
| | | ggcaagcccgagttcgaggaggccgacacccccgagaagctgcggaccctgctgg |
| | | ccgagaagctgtcctcccggcccgaggccgtgcacgagtacgtgacccccctgtt |
| | | cgtgtcccgggcccccaaccggaagatgtccggcgcccacaaggacaccctgcgg |
| | | tccgccaagcggttcgtgaagcacaacgagaagatctccgtgaagcgggtgtggc |
| | | tgaccgagatcaagctggccgacctggagaacatggtgaactacaagaacggccg |
| | | ggagatcgagctgtacgaggccctgaaggcccggctggaggcctacggcggcaac |
| | | gccaagcaggccttcgaccccaaggacaaccccttctacaagaagggcggccagc |
| | | tggtgaaggccgtgcgggtggagaagacccaggagtccggcgtgctgctgaacaa |
| | | gaagaacgcctacaccatcgccgacaacggcgacatggtgcgggtggacgtgttc |
| | | tgcaaggtggacaagaagggcaagaaccagtacttcatcgtgcccatctacgcct |
| | | ggcaggtggccgagaacatcctgcccgacatcgactgcaagggctaccggatcga |
| | | cgactcctacaccttctgcttctccctgcacaagtacgacctgatcgccttccag |
| | | aaggacgagaagtccaaggtggagttcgcctactacatcaactgcgactcctcca |
| | | acggccggttctacctggcctggcacgacaagggctccaaggagcagcagttccg |
| | | gatctccacccagaacctggtgctgatccagaagtaccaggtgaacgagctgggc |
| | | aaggagatccggccctgccggctgaagaagcggccccccgtgcggTCCGGAAAGC |
| | | GGACCGCCGACGGCTCCGGAGGAGGAAGCCCCAAGAAGAAGCGGAAGGTGtagct |
| | | agcaccagcctcaagaacacccgaatggagtctctaagctacataataccaactt |
| | | acactttacaaaatgttgtcccccaaaatgtagccattcgtatctgctcctaata |
| | | aaaagaaagtttcttcacattctctcgagAAAAAAAAAAAATGGAAAAAAAAAAA |
| | | ACGGAAAAAAAAAAAAGGTAAAAAAAAAAAATATAAAAAAAAAAAACATAAAAAA |
| | | AAAAAACGAAAAAAAAAAAACGTAAAAAAAAAAACTCAAAAAAAAAAAGATAAAA |
| | | AAAAAAAACCTAAAAAAAAAAAATGTAAAAAAAAAAAAGGGAAAAAAAAAAAACG |
| | | CAAAAAAAAAAAACACAAAAAAAAAAAATGCAAAAAAAAAAAATCGAAAAAAAAA |
| | | AAATCTAAAAAAAAAAAACGAAAAAAAAAAAACCCAAAAAAAAAAAAGACAAAAA |
| | | AAAAAAATAGAAAAAAAAAAAGTTAAAAAAAAAAAACTGAAAAAAAAAAAATTTA |
| | | AAAAAAAAAAAT |
| 630 | mRNA L encoding | GGGaagctcagaataaacgctcaactttggccggatctgccacCatgGACGGCTC |
| | Nme2Cas9 | CGGCGGCGGCTCCCCCAAGAAGAAGCGGAAGGTGGGCGGCTCCGGCGGCGGCgcc |
| | | gccttcaagcccaaccccatcaactacatcctgggcctggacatcggcatcgcct |
| | | ccgtgggctgggccatggtggagatcgacgaggaggagaaccccatccggctgat |
| | | cgacctgggcgtgcgggtgttcgagcgggccgaggtgcccaagaccggcgactcc |
| | | ctggccatggcccggcggctggcccggtccgtgcggcggctgacccggcggcggg |
| | | cccaccggctgctgcgggcccggcggctgctgaagcgggagggcgtgctgcaggc |
| | | cgccgacttcgacgagaacggcctgatcaagtccctgcccaacaccccctggcag |
| | | ctgcgggccgccgccctggaccggaagctgacccccctggagtggtccgccgtgc |
| | | tgctgcacctgatcaagcaccggggctacctgtcccagcggaagaacgagggcga |
| | | gaccgccgacaaggagctgggcgccctgctgaagggcgtggccaacaacgcccac |
| | | gccctgcagaccggcgacttccggacccccgccgagctggccctgaacaagttcg |
| | | agaaggagtccggccacatccggaaccagcggggcgactactcccacaccttctc |
| | | ccggaaggacctgcaggccgagctgatcctgctgttcgagaagcagaaggagttc |
| | | ggcaacccccacgtgtccggcggcctgaaggagggcatcgagaccctgctgatga |
| | | cccagcggcccgccctgtccggcgacgccgtgcagaagatgctgggccactgcac |
| | | cttcgagcccgccgagcccaaggccgccaagaacacctacaccgccgagcggttc |
| | | atctggctgaccaagctgaacaacctgcggatcctggagcagggctccgagcggc |
| | | ccctgaccgacaccgagcgggccaccctgatggacgagccctaccggaagtccaa |
| | | gctgacctacgcccaggcccggaagctgctgggcctggaggacaccgccttcttc |
| | | aagggcctgcggtacggcaaggacaacgccgaggcctccaccctgatggagatga |
| | | aggcctaccacgccatctcccgggccctggagaaggagggcctgaaggacaagaa |
| | | gtcccccctgaacctgtcctccgagctgcaggacgagatcggcaccgccttctcc |
| | | ctgttcaagaccgacgaggacatcaccggccggctgaaggaccgggtgcagcccg |
| | | agatcctggaggccctgctgaagcacatctccttcgacaagttcgtgcagatctc |
| | | cctgaaggccctgcggcggatcgtgcccctgatggagcagggcaagcggtacgac |
| | | gaggcctgcgccgagatctacggcgaccactacggcaagaagaacaccgaggaga |
| | | agatctacctgccccccatccccgccgacgagatccggaaccccgtggtgctgcg |
| | | ggccctgtcccaggcccggaaggtgatcaacggcgtggtgcggcggtacggctcc |
| | | cccgcccggatccacatcgagaccgcccgggaggtgggcaagtccttcaaggacc |
| | | ggaaggagatcgagaagcggcaggaggagaaccggaaggaccgggagaaggccgc |
| | | cgccaagttccgggagtacttccccaacttcgtgggcgagcccaagtccaaggac |
| | | atcctgaagctgcggctgtacgagcagcagcacggcaagtgcctgtactccggca |
| | | aggagatcaacctggtgcggctgaacgagaagggctacgtggagatcgaccacgc |
| | | cctgcccttctcccggacctgggacgactccttcaacaacaaggtgctggtgctg |
| | | ggctccgagaaccagaacaagggcaaccagaccccctacgagtacttcaacggca |
| | | aggacaactcccgggagtggcaggagttcaaggcccgggtggagacctcccggtt |
| | | cccccggtccaagaagcagcggatcctgctgcagaagttcgacgaggacggcttc |
| | | aaggagtgcaacctgaacgacacccggtacgtgaaccggttcctgtgccagttcg |
| | | tggccgaccacatcctgctgaccggcaagggcaagcggcgggtgttcgcctccaa |
| | | cggccagatcaccaacctgctgcggggcttctggggcctgcggaaggtgcgggcc |
| | | gagaacgaccggcaccacgccctggacgccgtggtggtggcctgctccaccgtgg |
| | | ccatgcagcagaagatcacccggttcgtgcggtacaaggagatgaacgccttcga |
| | | cggcaagaccatcgacaaggagaccggcaaggtgctgcaccagaagacccacttc |
| | | ccccagccctgggagttcttcgcccaggaggtgatgatccgggtgttcggcaagc |
| | | ccgacggcaagcccgagttcgaggaggccgacacccccgagaagctgcggaccct |
| | | gctggccgagaagctgtcctcccggcccgaggccgtgcacgagtacgtgaccccc |
| | | ctgttcgtgtcccgggcccccaaccggaagatgtccggcgcccacaaggacaccc |
| | | tgcggtccgccaagcggttcgtgaagcacaacgagaagatctccgtgaagcgggt |
| | | gtggctgaccgagatcaagctggccgacctggagaacatggtgaactacaagaac |
| | | ggccgggagatcgagctgtacgaggccctgaaggcccggctggaggcctacggcg |
| | | gcaacgccaagcaggccttcgaccccaaggacaaccccttctacaagaagggcgg |
| | | ccagctggtgaaggccgtgcgggtggagaagacccaggagtccggcgtgctgctg |
| | | aacaagaagaacgcctacaccatcgccgacaacggcgacatggtgcgggggacgt |
| | | gttctgcaaggtggacaagaagggcaagaaccagtacttcatcgtgcccatctac |
| | | gcctggcaggtggccgagaacatcctgcccgacatcgactgcaagggctaccgga |
| | | tcgacgactcctacaccttctgcttctccctgcacaagtacgacctgatcgcctt |
| | | ccagaaggacgagaagtccaaggtggagttcgcctactacatcaactgcgactcc |
| | | tccaacggccggttctacctggcctggcacgacaagggctccaaggagcagcagt |
| | | tccggatctccacccagaacctggtgctgatccagaagtaccaggtgaacgagct |
| | | gggcaaggagatccggccctgccggctgaagaagcggccccccgtgcggtagcta |
| | | gcaccagcctcaagaacacccgaatggagtctctaagctacataataccaactta |
| | | cactttacaaaatgttgtcccccaaaatgtagccattcgtatctgctcctaataa |
| | | aaagaaagtttcttcacattctctcgagAAAAAAAAAAAATGGAAAAAAAAAAAA |
| | | CGGAAAAAAAAAAAAGGTAAAAAAAAAAAATATAAAAAAAAAAAACATAAAAAAA |
| | | AAAAACGAAAAAAAAAAAACGTAAAAAAAAAAAACTCAAAAAAAAAAAGATAAAA |
| | | AAAAAAAACCTAAAAAAAAAAAATGTAAAAAAAAAAAAGGGAAAAAAAAAAAACG |
| | | CAAAAAAAAAAAACACAAAAAAAAAAAATGCAAAAAAAAAAAATCGAAAAAAAAA |
| | | AAATCTAAAAAAAAAAAACGAAAAAAAAAAAACCCAAAAAAAAAAAAGACAAAAA |
| | | AAAAAAATAGAAAAAAAAAAAGTTAAAAAAAAAAAACTGAAAAAAAAAAAATTTA |
| | | AAAAAAAAAAAT |
| 631 | mRNA M encoding | GGGAAGCUCAGAAUAAACGCUCAACUUUGGCCGGAUCUGCCACCAUGGACGGCUC |
| | Nme2Cas9 with | CGGCGGCGGCUCCCCCAAGAAGAAGCGGAAGGUGGGCGGCUCCGGCGGCGGCGCC |
| | HiBiT tag | GCCUUCAAGCCCAACCCCAUCAACUACAUCCUGGGCCUGGACAUCGGCAUCGCCU |
| | | CCGUGGGCUGGGCCAUGGUGGAGAUCGACGAGGAGGAGAACCCCAUCCGGCUGAU |
| | | CGACCUGGGCGUGCGGGUGUUCGAGCGGGCCGAGGUGCCCAAGACCGGCGACUCC |
| | | CUGGCCAUGGCCCGGCGGCUGGCCCGGUCCGUGCGGCGGCUGACCCGGCGGCGGG |
| | | CCCACCGGCUGCUGCGGGCCCGGCGGCUGCUGAAGCGGGAGGGCGUGCUGCAGGC |
| | | CGCCGACUUCGACGAGAACGGCCUGAUCAAGUCCCUGCCCAACACCCCCUGGCAG |
| | | CUGCGGGCCGCCGCCCUGGACCGGAAGCUGACCCCCCUGGAGUGGUCCGCCGUGC |
| | | UGCUGCACCUGAUCAAGCACCGGGGCUACCUGUCCCAGCGGAAGAACGAGGGCGA |
| | | GACCGCCGACAAGGAGCUGGGCGCCCUGCUGAAGGGCGUGGCCAACAACGCCCAC |
| | | GCCCUGCAGACCGGCGACUUCCGGACCCCCGCCGAGCUGGCCCUGAACAAGUUCG |
| | | AGAAGGAGUCCGGCCACAUCCGGAACCAGCGGGGCGACUACUCCCACACCUUCUC |
| | | CCGGAAGGACCUGCAGGCCGAGCUGAUCCUGCUGUUCGAGAAGCAGAAGGAGUUC |
| | | GGCAACCCCCACGUGUCCGGCGGCCUGAAGGAGGGCAUCGAGACCCUGCUGAUGA |
| | | CCCAGCGGCCCGCCCUGUCCGGCGACGCCGUGCAGAAGAUGCUGGGCCACUGCAC |
| | | CUUCGAGCCCGCCGAGCCCAAGGCCGCCAAGAACACCUACACCGCCGAGCGGUUC |
| | | AUCUGGCUGACCAAGCUGAACAACCUGCGGAUCCUGGAGCAGGGCUCCGAGCGGC |
| | | CCCUGACCGACACCGAGCGGGCCACCCUGAUGGACGAGCCCUACCGGAAGUCCAA |
| | | GCUGACCUACGCCCAGGCCCGGAAGCUGCUGGGCCUGGAGGACACCGCCUUCUUC |
| | | AAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAA |
| | | GUCCCCCCUGAACCUGUCCUCCGAGCUGCAGGACGAGAUCGGCACCGCCUUCUCC |
| | | CUGUUCAAGACCGACGAGGACAUCACCGGCCGGCUGAAGGACCGGGUGCAGCCCG |
| | | AGAUCCUGGAGGCCCUGCUGAAGCACAUCUCCUUCGACAAGUUCGUGCAGAUCUC |
| | | CCUGAAGGCCCUGCGGCGGAUCGUGCCCCUGAUGGAGCAGGGCAAGCGGUACGAC |
| | | GAGGCCUGCGCCGAGAUCUACGGCGACCACUACGGCAAGAAGAACACCGAGGAGA |
| | | AGAUCUACCUGCCCCCCAUCCCCGCCGACGAGAUCCGGAACCCCGUGGUGCUGCG |
| | | GGCCCUGUCCCAGGCCCGGAAGGUGAUCAACGGCGUGGUGCGGCGGUACGGCUCC |
| | | CCCGCCCGGAUCCACAUCGAGACCGCCCGGGAGGUGGGCAAGUCCUUCAAGGACC |
| | | GGAAGGAGAUCGAGAAGCGGCAGGAGGAGAACCGGAAGGACCGGGAGAAGGCCGC |
| | | CGCCAAGUUCCGGGAGUACUUCCCCAACUUCGUGGGCGAGCCCAAGUCCAAGGAC |
| | | AUCCUGAAGCUGCGGCUGUACGAGCAGCAGCACGGCAAGUGCCUGUACUCCGGCA |
| | | AGGAGAUCAACCUGGUGCGGCUGAACGAGAAGGGCUACGUGGAGAUCGACCACGC |
| | | CCUGCCCUUCUCCCGGACCUGGGACGACUCCUUCAACAACAAGGUGCUGGUGCUG |
| | | GGCUCCGAGAACCAGAACAAGGGCAACCAGACCCCCUACGAGUACUUCAACGGCA |
| | | AGGACAACUCCCGGGAGUGGCAGGAGUUCAAGGCCCGGGUGGAGACCUCCCGGUU |
| | | CCCCCGGUCCAAGAAGCAGCGGAUCCUGCUGCAGAAGUUCGACGAGGACGGCUUC |
| | | AAGGAGUGCAACCUGAACGACACCCGGUACGUGAACCGGUUCCUGUGCCAGUUCG |
| | | UGGCCGACCACAUCCUGCUGACCGGCAAGGGCAAGCGGCGGGUGUUCGCCUCCAA |
| | | CGGCCAGAUCACCAACCUGCUGCGGGGCUUCUGGGGCCUGCGGAAGGUGCGGGCC |
| | | GAGAACGACCGGCACCACGCCCUGGACGCCGUGGUGGUGGCCUGCUCCACCGUGG |
| | | CCAUGCAGCAGAAGAUCACCCGGUUCGUGCGGUACAAGGAGAUGAACGCCUUCGA |
| | | CGGCAAGACCAUCGACAAGGAGACCGGCAAGGUGCUGCACCAGAAGACCCACUUC |
| | | CCCCAGCCCUGGGAGUUCUUCGCCCAGGAGGUGAUGAUCCGGGUGUUCGGCAAGC |
| | | CCGACGGCAAGCCCGAGUUCGAGGAGGCCGACACCCCCGAGAAGCUGCGGACCCU |
| | | GCUGGCCGAGAAGCUGUCCUCCCGGCCCGAGGCCGUGCACGAGUACGUGACCCCC |
| | | CUGUUCGUGUCCCGGGCCCCCAACCGGAAGAUGUCCGGCGCCCACAAGGACACCC |
| | | UGCGGUCCGCCAAGCGGUUCGUGAAGCACAACGAGAAGAUCUCCGUGAAGCGGGU |
| | | GUGGCUGACCGAGAUCAAGCUGGCCGACCUGGAGAACAUGGUGAACUACAAGAAC |
| | | GGCCGGGAGAUCGAGCUGUACGAGGCCCUGAAGGCCCGGCUGGAGGCCUACGGCG |
| | | GCAACGCCAAGCAGGCCUUCGACCCCAAGGACAACCCCUUCUACAAGAAGGGCGG |
| | | CCAGCUGGUGAAGGCCGUGCGGGUGGAGAAGACCCAGGAGUCCGGCGUGCUGCUG |
| | | AACAAGAAGAACGCCUACACCAUCGCCGACAACGGCGACAUGGUGCGGGUGGACG |
| | | UGUUCUGCAAGGUGGACAAGAAGGGCAAGAACCAGUACUUCAUCGUGCCCAUCUA |
| | | CGCCUGGCAGGUGGCCGAGAACAUCCUGCCCGACAUCGACUGCAAGGGCUACCGG |
| | | AUCGACGACUCCUACACCUUCUGCUUCUCCCUGCACAAGUACGACCUGAUCGCCU |
| | | UCCAGAAGGACGAGAAGUCCAAGGUGGAGUUCGCCUACUACAUCAACUGCGACUC |
| | | CUCCAACGGCCGGUUCUACCUGGCCUGGCACGACAAGGGCUCCAAGGAGCAGCAG |
| | | UUCCGGAUCUCCACCCAGAACCUGGUGCUGAUCCAGAAGUACCAGGUGAACGAGC |
| | | UGGGCAAGGAGAUCCGGCCCUGCCGGCUGAAGAAGCGGCCCCCCGUGCGGUCCGA |
| | | GUCCGCCACCCCCGAGUCCGUGUCCGGCUGGCGGCUGUUCAAGAAGAUCUCCUAG |
| | | CUAGCACCAGCCUCAAGAACACCCGAAUGGAGUCUCUAAGCUACAUAAUACCAAC |
| | | UUACACUUUACAAAAUGUUGUCCCCCAAAAUGUAGCCAUUCGUAUCUGCUCCUAA |
| | | UAAAAAGAAAGUUUCUUCACAUUCUCUCGAGAAAAAAAAAAAAUGGAAAAAAAAA |
| | | AAACGGAAAAAAAAAAAAGGUAAAAAAAAAAAAUAUAAAAAAAAAAAACAUAAAA |
| | | AAAAAAAACGAAAAAAAAAAAACGUAAAAAAAAAAAACUCAAAAAAAAAAAAGAU |
| | | AAAAAAAAAAAACCUAAAAAAAAAAAAUGUAAAAAAAAAAAAGGGAAAAAAAAAA |
| | | AACGCAAAAAAAAAAAACACAAAAAAAAAAAAUGCAAAAAAAAAAAAUCGAAAAA |
| | | AAAAAAAUCUAAAAAAAAAAAACGAAAAAAAAAAAACCCAAAAAAAAAAAAGACA |
| | | AAAAAAAAAAAUAGAAAAAAAAAAAAGUUAAAAAAAAAAAACUGAAAAAAAAAAA |
| | | AUUUAAAAAAAAAAAAUCUAG |
| 632 | mRNA N encoding | GGGAAGCUCAGAAUAAACGCUCAACUUUGGCCGGAUCUGCCACCAUGGACGGCUC |
| | Nme2Cas 9 | CGGCGGCGGCUCCCCCAAGAAGAAGCGGAAGGUGGGCGGCUCCGGCGGCGGCGCC |
| | | GCCUUCAAGCCCAACCCCAUCAACUACAUCCUGGGCCUGGACAUCGGCAUCGCCU |
| | | CCGUGGGCUGGGCCAUGGUGGAGAUCGACGAGGAGGAGAACCCCAUCCGGCUGAU |
| | | CGACCUGGGCGUGCGGGUGUUCGAGCGGGCCGAGGUGCCCAAGACCGGCGACUCC |
| | | CUGGCCAUGGCCCGGCGGCUGGCCCGGUCCGUGCGGCGGCUGACCCGGCGGCGGG |
| | | CCCACCGGCUGCUGCGGGCCCGGCGGCUGCUGAAGCGGGAGGGCGUGCUGCAGGC |
| | | CGCCGACUUCGACGAGAACGGCCUGAUCAAGUCCCUGCCCAACACCCCCUGGCAG |
| | | CUGCGGGCCGCCGCCCUGGACCGGAAGCUGACCCCCCUGGAGUGGUCCGCCGUGC |
| | | UGCUGCACCUGAUCAAGCACCGGGGCUACCUGUCCCAGCGGAAGAACGAGGGCGA |
| | | GACCGCCGACAAGGAGCUGGGCGCCCUGCUGAAGGGCGUGGCCAACAACGCCCAC |
| | | GCCCUGCAGACCGGCGACUUCCGGACCCCCGCCGAGCUGGCCCUGAACAAGUUCG |
| | | AGAAGGAGUCCGGCCACAUCCGGAACCAGCGGGGCGACUACUCCCACACCUUCUC |
| | | CCGGAAGGACCUGCAGGCCGAGCUGAUCCUGCUGUUCGAGAAGCAGAAGGAGUUC |
| | | GGCAACCCCCACGUGUCCGGCGGCCUGAAGGAGGGCAUCGAGACCCUGCUGAUGA |
| | | CCCAGCGGCCCGCCCUGUCCGGCGACGCCGUGCAGAAGAUGCUGGGCCACUGCAC |
| | | CUUCGAGCCCGCCGAGCCCAAGGCCGCCAAGAACACCUACACCGCCGAGCGGUUC |
| | | AUCUGGCUGACCAAGCUGAACAACCUGCGGAUCCUGGAGCAGGGCUCCGAGCGGC |
| | | CCCUGACCGACACCGAGCGGGCCACCCUGAUGGACGAGCCCUACCGGAAGUCCAA |
| | | GCUGACCUACGCCCAGGCCCGGAAGCUGCUGGGCCUGGAGGACACCGCCUUCUUC |
| | | AAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAA |
| | | GUCCCCCCUGAACCUGUCCUCCGAGCUGCAGGACGAGAUCGGCACCGCCUUCUCC |
| | | CUGUUCAAGACCGACGAGGACAUCACCGGCCGGCUGAAGGACCGGGUGCAGCCCG |
| | | AGAUCCUGGAGGCCCUGCUGAAGCACAUCUCCUUCGACAAGUUCGUGCAGAUCUC |
| | | CCUGAAGGCCCUGCGGCGGAUCGUGCCCCUGAUGGAGCAGGGCAAGCGGUACGAC |
| | | GAGGCCUGCGCCGAGAUCUACGGCGACCACUACGGCAAGAAGAACACCGAGGAGA |
| | | AGAUCUACCUGCCCCCCAUCCCCGCCGACGAGAUCCGGAACCCCGUGGUGCUGCG |
| | | GGCCCUGUCCCAGGCCCGGAAGGUGAUCAACGGCGUGGUGCGGCGGUACGGCUCC |
| | | CCCGCCCGGAUCCACAUCGAGACCGCCCGGGAGGUGGGCAAGUCCUUCAAGGACC |
| | | GGAAGGAGAUCGAGAAGCGGCAGGAGGAGAACCGGAAGGACCGGGAGAAGGCCGC |
| | | CGCCAAGUUCCGGGAGUACUUCCCCAACUUCGUGGGCGAGCCCAAGUCCAAGGAC |
| | | AUCCUGAAGCUGCGGCUGUACGAGCAGCAGCACGGCAAGUGCCUGUACUCCGGCA |
| | | AGGAGAUCAACCUGGUGCGGCUGAACGAGAAGGGCUACGUGGAGAUCGACCACGC |
| | | CCUGCCCUUCUCCCGGACCUGGGACGACUCCUUCAACAACAAGGUGCUGGUGCUG |
| | | GGCUCCGAGAACCAGAACAAGGGCAACCAGACCCCCUACGAGUACUUCAACGGCA |
| | | AGGACAACUCCCGGGAGUGGCAGGAGUUCAAGGCCCGGGUGGAGACCUCCCGGUU |
| | | CCCCCGGUCCAAGAAGCAGCGGAUCCUGCUGCAGAAGUUCGACGAGGACGGCUUC |
| | | AAGGAGUGCAACCUGAACGACACCCGGUACGUGAACCGGUUCCUGUGCCAGUUCG |
| | | UGGCCGACCACAUCCUGCUGACCGGCAAGGGCAAGCGGCGGGUGUUCGCCUCCAA |
| | | CGGCCAGAUCACCAACCUGCUGCGGGGCUUCUGGGGCCUGCGGAAGGUGCGGGCC |
| | | GAGAACGACCGGCACCACGCCCUGGACGCCGUGGUGGUGGCCUGCUCCACCGUGG |
| | | CCAUGCAGCAGAAGAUCACCCGGUUCGUGCGGUACAAGGAGAUGAACGCCUUCGA |
| | | CGGCAAGACCAUCGACAAGGAGACCGGCAAGGUGCUGCACCAGAAGACCCACUUC |
| | | CCCCAGCCCUGGGAGUUCUUCGCCCAGGAGGUGAUGAUCCGGGUGUUCGGCAAGC |
| | | CCGACGGCAAGCCCGAGUUCGAGGAGGCCGACACCCCCGAGAAGCUGCGGACCCU |
| | | GCUGGCCGAGAAGCUGUCCUCCCGGCCCGAGGCCGUGCACGAGUACGUGACCCCC |
| | | CUGUUCGUGUCCCGGGCCCCCAACCGGAAGAUGUCCGGCGCCCACAAGGACACCC |
| | | UGCGGUCCGCCAAGCGGUUCGUGAAGCACAACGAGAAGAUCUCCGUGAAGCGGGU |
| | | GUGGCUGACCGAGAUCAAGCUGGCCGACCUGGAGAACAUGGUGAACUACAAGAAC |
| | | GGCCGGGAGAUCGAGCUGUACGAGGCCCUGAAGGCCCGGCUGGAGGCCUACGGCG |
| | | GCAACGCCAAGCAGGCCUUCGACCCCAAGGACAACCCCUUCUACAAGAAGGGCGG |
| | | CCAGCUGGUGAAGGCCGUGCGGGUGGAGAAGACCCAGGAGUCCGGCGUGCUGCUG |
| | | AACAAGAAGAACGCCUACACCAUCGCCGACAACGGCGACAUGGUGCGGGUGGACG |
| | | UGUUCUGCAAGGUGGACAAGAAGGGCAAGAACCAGUACUUCAUCGUGCCCAUCUA |
| | | CGCCUGGCAGGUGGCCGAGAACAUCCUGCCCGACAUCGACUGCAAGGGCUACCGG |
| | | AUCGACGACUCCUACACCUUCUGCUUCUCCCUGCACAAGUACGACCUGAUCGCCU |
| | | UCCAGAAGGACGAGAAGUCCAAGGUGGAGUUCGCCUACUACAUCAACUGCGACUC |
| | | CUCCAACGGCCGGUUCUACCUGGCCUGGCACGACAAGGGCUCCAAGGAGCAGCAG |
| | | UUCCGGAUCUCCACCCAGAACCUGGUGCUGAUCCAGAAGUACCAGGUGAACGAGC |
| | | UGGGCAAGGAGAUCCGGCCCUGCCGGCUGAAGAAGCGGCCCCCCGUGCGGUCCGG |
| | | AAAGCGGACCGCCGACGGCUCCGGAGGAGGAAGCCCCGCCGCCAAGAAGAAGAAG |
| | | CUGGACUAGCUAGCACCAGCCUCAAGAACACCCGAAUGGAGUCUCUAAGCUACAU |
| | | AAUACCAACUUACACUUUACAAAAUGUUGUCCCCCAAAAUGUAGCCAUUCGUAUC |
| | | UGCUCCUAAUAAAAAGAAAGUUUCUUCACAUUCUCUCGAGAAAAAAAAAAAAUGG |
| | | AAAAAAAAAAAACGGAAAAAAAAAAAAGGUAAAAAAAAAAAAUAUAAAAAAAAAA |
| | | AACAUAAAAAAAAAAAACGAAAAAAAAAAAACGUAAAAAAAAAAAACUCAAAAAA |
| | | AAAAAGAUAAAAAAAAAAAACCUAAAAAAAAAAAAUGUAAAAAAAAAAAAGGGAA |
| | | AAAAAAAAAACGCAAAAAAAAAAAACACAAAAAAAAAAAAUGCAAAAAAAAAAAA |
| | | UCGAAAAAAAAAAAAUCUAAAAAAAAAAAACGAAAAAAAAAAAACCCAAAAAAAA |
| | | AAAAGACAAAAAAAAAAAAUAGAAAAAAAAAAAGUUAAAAAAAAAAAACUGAAAA |
| | | AAAAAAAAUUUAAAAAAAAAAAAUCUAG |
| 633 | mRNA O encoding | GGGAAGCUCAGAAUAAACGCUCAACUUUGGCCGGAUCUGCCACCAUGGACGGCUC |
| | Nme 2Cas 9 | CGGCGGCGGCUCCCCCAAGAAGAAGCGGAAGGUGGAGGACAAGCGGCCCGCCGCC |
| | | ACCAAGAAGGCCGGCCAGGCCAAGAAGAAGAAGGGCGGCUCCGGCGGCGGCGCCG |
| | | CCUUCAAGCCCAACCCCAUCAACUACAUCCUGGGCCUGGACAUCGGCAUCGCCUC |
| | | CGUGGGCUGGGCCAUGGUGGAGAUCGACGAGGAGGAGAACCCCAUCCGGCUGAUC |
| | | GACCUGGGCGUGCGGGUGUUCGAGCGGGCCGAGGUGCCCAAGACCGGCGACUCCC |
| | | UGGCCAUGGCCCGGCGGCUGGCCCGGUCCGUGCGGCGGCUGACCCGGCGGCGGGC |
| | | CCACCGGCUGCUGCGGGCCCGGCGGCUGCUGAAGCGGGAGGGCGUGCUGCAGGCC |
| | | GCCGACUUCGACGAGAACGGCCUGAUCAAGUCCCUGCCCAACACCCCCUGGCAGC |
| | | UGCGGGCCGCCGCCCUGGACCGGAAGCUGACCCCCCUGGAGUGGUCCGCCGUGCU |
| | | GCUGCACCUGAUCAAGCACCGGGGCUACCUGUCCCAGCGGAAGAACGAGGGCGAG |
| | | ACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGA |
| | | GAAGGAGUCCGGCCACAUCCGGAACCAGCGGGGCGACUACUCCCACACCUUCUCC |
| | | CGGAAGGACCUGCAGGCCGAGCUGAUCCUGCUGUUCGAGAAGCAGAAGGAGUUCG |
| | | GCAACCCCCACGUGUCCGGCGGCCUGAAGGAGGGCAUCGAGACCCUGCUGAUGAC |
| | | CCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCA |
| | | UCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAG |
| | | CUGACCUACGCCCAGGCCCGGAAGCUGCUGGGCCUGGAGGACACCGCCUUCUUCA |
| | | AGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAG |
| | | UCCCCCCUGAACCUGUCCUCCGAGCUGCAGGACGAGAUCGGCACCGCCUUCUCCC |
| | | UGUUCAAGACCGACGAGGACAUCACCGGCCGGCUGAAGGACCGGGUGCAGCCCGA |
| | | GAUCCUGGAGGCCCUGCUGAAGCACAUCUCCUUCGACAAGUUCGUGCAGAUCUCC |
| | | CUGAAGGCCCUGCGGCGGAUCGUGCCCCUGAUGGAGCAGGGCAAGCGGUACGACG |
| | | AGGCCUGCGCCGAGAUCUACGGCGACCACUACGGCAAGAAGAACACCGAGGAGAA |
| | | GAUCUACCUGCCCCCCAUCCCCGCCGACGAGAUCCGGAACCCCGUGGUGCUGCGG |
| | | GCCCUGUCCCAGGCCCGGAAGGUGAUCAACGGCGUGGUGCGGCGGUACGGCUCCC |
| | | CCGCCCGGAUCCACAUCGAGACCGCCCGGGAGGUGGGCAAGUCCUUCAAGGACCG |
| | | GAAGGAGAUCGAGAAGCGGCAGGAGGAGAACCGGAAGGACCGGGAGAAGGCCGCC |
| | | GCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCC |
| | | CUGCCCUUCUCCCGGACCUGGGACGACUCCUUCAACAACAAGGUGCUGGUGCUGG |
| | | GCUCCGAGAACCAGAACAAGGGCAACCAGACCCCCUACGAGUACUUCAACGGCAA |
| | | GGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCA |
| | | AGGAGUGCAACCUGAACGACACCCGGUACGUGAACCGGUUCCUGUGCCAGUUCGU |
| | | GGCCGACCACAUCCUGCUGACCGGCAAGGGCAAGCGGCGGGUGUUCGCCUCCAAC |
| | | GGCCAGAUCACCAACCUGCUGCGGGGCUUCUGGGGCCUGCGGAAGGUGCGGGCCG |
| | | AGAACGACCGGCACCACGCCCUGGACGCCGUGGUGGUGGCCUGCUCCACCGUGGC |
| | | CAUGCAGCAGAAGAUCACCCGGUUCGUGCGGUACAAGGAGAUGAACGCCUUCGAC |
| | | GGCAAGACCAUCGACAAGGAGACCGGCAAGGUGCUGCACCAGAAGACCCACUUCC |
| | | CCCAGCCCUGGGAGUUCUUCGCCCAGGAGGUGAUGAUCCGGGUGUUCGGCAAGCC |
| | | CGACGGCAAGCCCGAGUUCGAGGAGGCCGACACCCCCGAGAAGCUGCGGACCCUG |
| | | CUGGCCGAGAAGCUGUCCUCCCGGCCCGAGGCCGUGCACGAGUACGUGACCCCCC |
| | | UGUUCGUGUCCCGGGCCCCCAACCGGAAGAUGUCCGGCGCCCACAAGGACACCCU |
| | | GCGGUCCGCCAAGCGGUUCGUGAAGCACAACGAGAAGAUCUCCGUGAAGCGGGUG |
| | | UGGCUGACCGAGAUCAAGCUGGCCGACCUGGAGAACAUGGUGAACUACAAGAACG |
| | | GCCGGGAGAUCGAGCUGUACGAGGCCCUGAAGGCCCGGCUGGAGGCCUACGGCGG |
| | | CAACGCCAAGCAGGCCUUCGACCCCAAGGACAACCCCUUCUACAAGAAGGGCGGC |
| | | CAGCUGGUGAAGGCCGUGCGGGUGGAGAAGACCCAGGAGUCCGGCGUGCUGCUGA |
| | | ACAAGAAGAACGCCUACACCAUCGCCGACAACGGCGACAUGGUGCGGGUGGACGU |
| | | GUUCUGCAAGGUGGACAAGAAGGGCAAGAACCAGUACUUCAUCGUGCCCAUCUAC |
| | | GCCUGGCAGGUGGCCGAGAACAUCCUGCCCGACAUCGACUGCAAGGGCUACCGGA |
| | | UCGACGACUCCUACACCUUCUGCUUCUCCCUGCACAAGUACGACCUGAUCGCCUU |
| | | CCAGAAGGACGAGAAGUCCAAGGUGGAGUUCGCCUACUACAUCAACUGCGACUCC |
| | | UCCAACGGCCGGUUCUACCUGGCCUGGCACGACAAGGGCUCCAAGGAGCAGCAGU |
| | | UCCGGAUCUCCACCCAGAACCUGGUGCUGAUCCAGAAGUACCAGGUGAACGAGCU |
| | | GGGCAAGGAGAUCCGGCCCUGCCGGCUGAAGAAGCGGCCCCCCGUGCGGUAGCUA |
| | | GCACCAGCCUCAAGAACACCCGAAUGGAGUCUCUAAGCUACAUAAUACCAACUUA |
| | | CACUUUACAAAAUGUUGUCCCCCAAAAUGUAGCCAUUCGUAUCUGCUCCUAAUAA |
| | | AAAGAAAGUUUCUUCACAUUCUCUCGAGAAAAAAAAAAAAUGGAAAAAAAAAAAA |
| | | CGGAAAAAAAAAAAAGGUAAAAAAAAAAAAUAUAAAAAAAAAAAACAUAAAAAAA |
| | | AAAAACGAAAAAAAAAAAACGUAAAAAAAAAAAACUCAAAAAAAAAAAGAUAAAA |
| | | AAAAAAAACCUAAAAAAAAAAAAUGUAAAAAAAAAAAAGGGAAAAAAAAAAAACG |
| | | CAAAAAAAAAAAACACAAAAAAAAAAAAUGCAAAAAAAAAAAAUCGAAAAAAAAA |
| | | AAAUCUAAAAAAAAAAAACGAAAAAAAAAAAACCCAAAAAAAAAAAAGACAAAAA |
| | | AAAAAAAUAGAAAAAAAAAAAGUUAAAAAAAAAAAACUGAAAAAAAAAAAAUUUA |
| | | AAAAAAAAAAAUCUAG |
| 634 | mRNA P encoding | GGGAAGCUCAGAAUAAACGCUCAACUUUGGCCGGAUCUGCCACCAUGGACGGCUC |
| | Nme2Cas9 with | CGGCGGCGGCUCCCCCAAGAAGAAGCGGAAGGUGGAGGACAAGCGGCCCGCCGCC |
| | HiBiT tag | ACCAAGAAGGCCGGCCAGGCCAAGAAGAAGAAGGGCGGCUCCGGCGGCGGCGCCG |
| | | CCUUCAAGCCCAACCCCAUCAACUACAUCCUGGGCCUGGACAUCGGCAUCGCCUC |
| | | CGUGGGCUGGGCCAUGGUGGAGAUCGACGAGGAGGAGAACCCCAUCCGGCUGAUC |
| | | GACCUGGGCGUGCGGGUGUUCGAGCGGGCCGAGGUGCCCAAGACCGGCGACUCCC |
| | | UGGCCAUGGCCCGGCGGCUGGCCCGGUCCGUGCGGCGGCUGACCCGGCGGCGGGC |
| | | CCACCGGCUGCUGCGGGCCCGGCGGCUGCUGAAGCGGGAGGGCGUGCUGCAGGCC |
| | | GCCGACUUCGACGAGAACGGCCUGAUCAAGUCCCUGCCCAACACCCCCUGGCAGC |
| | | UGCGGGCCGCCGCCCUGGACCGGAAGCUGACCCCCCUGGAGUGGUCCGCCGUGCU |
| | | GCUGCACCUGAUCAAGCACCGGGGCUACCUGUCCCAGCGGAAGAACGAGGGCGAG |
| | | ACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGA |
| | | GAAGGAGUCCGGCCACAUCCGGAACCAGCGGGGCGACUACUCCCACACCUUCUCC |
| | | CGGAAGGACCUGCAGGCCGAGCUGAUCCUGCUGUUCGAGAAGCAGAAGGAGUUCG |
| | | GCAACCCCCACGUGUCCGGCGGCCUGAAGGAGGGCAUCGAGACCCUGCUGAUGAC |
| | | CCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCA |
| | | UCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAG |
| | | CUGACCUACGCCCAGGCCCGGAAGCUGCUGGGCCUGGAGGACACCGCCUUCUUCA |
| | | AGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAG |
| | | UCCCCCCUGAACCUGUCCUCCGAGCUGCAGGACGAGAUCGGCACCGCCUUCUCCC |
| | | UGUUCAAGACCGACGAGGACAUCACCGGCCGGCUGAAGGACCGGGUGCAGCCCGA |
| | | GAUCCUGGAGGCCCUGCUGAAGCACAUCUCCUUCGACAAGUUCGUGCAGAUCUCC |
| | | CUGAAGGCCCUGCGGCGGAUCGUGCCCCUGAUGGAGCAGGGCAAGCGGUACGACG |
| | | AGGCCUGCGCCGAGAUCUACGGCGACCACUACGGCAAGAAGAACACCGAGGAGAA |
| | | GAUCUACCUGCCCCCCAUCCCCGCCGACGAGAUCCGGAACCCCGUGGUGCUGCGG |
| | | GCCCUGUCCCAGGCCCGGAAGGUGAUCAACGGCGUGGUGCGGCGGUACGGCUCCC |
| | | CCGCCCGGAUCCACAUCGAGACCGCCCGGGAGGUGGGCAAGUCCUUCAAGGACCG |
| | | GAAGGAGAUCGAGAAGCGGCAGGAGGAGAACCGGAAGGACCGGGAGAAGGCCGCC |
| | | GCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCC |
| | | CUGCCCUUCUCCCGGACCUGGGACGACUCCUUCAACAACAAGGUGCUGGUGCUGG |
| | | GCUCCGAGAACCAGAACAAGGGCAACCAGACCCCCUACGAGUACUUCAACGGCAA |
| | | GGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCA |
| | | AGGAGUGCAACCUGAACGACACCCGGUACGUGAACCGGUUCCUGUGCCAGUUCGU |
| | | GGCCGACCACAUCCUGCUGACCGGCAAGGGCAAGCGGCGGGUGUUCGCCUCCAAC |
| | | GGCCAGAUCACCAACCUGCUGCGGGGCUUCUGGGGCCUGCGGAAGGUGCGGGCCG |
| | | AGAACGACCGGCACCACGCCCUGGACGCCGUGGUGGUGGCCUGCUCCACCGUGGC |
| | | CAUGCAGCAGAAGAUCACCCGGUUCGUGCGGUACAAGGAGAUGAACGCCUUCGAC |
| | | GGCAAGACCAUCGACAAGGAGACCGGCAAGGUGCUGCACCAGAAGACCCACUUCC |
| | | CCCAGCCCUGGGAGUUCUUCGCCCAGGAGGUGAUGAUCCGGGUGUUCGGCAAGCC |
| | | CGACGGCAAGCCCGAGUUCGAGGAGGCCGACACCCCCGAGAAGCUGCGGACCCUG |
| | | CUGGCCGAGAAGCUGUCCUCCCGGCCCGAGGCCGUGCACGAGUACGUGACCCCCC |
| | | UGUUCGUGUCCCGGGCCCCCAACCGGAAGAUGUCCGGCGCCCACAAGGACACCCU |
| | | GCGGUCCGCCAAGCGGUUCGUGAAGCACAACGAGAAGAUCUCCGUGAAGCGGGUG |
| | | UGGCUGACCGAGAUCAAGCUGGCCGACCUGGAGAACAUGGUGAACUACAAGAACG |
| | | GCCGGGAGAUCGAGCUGUACGAGGCCCUGAAGGCCCGGCUGGAGGCCUACGGCGG |
| | | CAACGCCAAGCAGGCCUUCGACCCCAAGGACAACCCCUUCUACAAGAAGGGCGGC |
| | | CAGCUGGUGAAGGCCGUGCGGGUGGAGAAGACCCAGGAGUCCGGCGUGCUGCUGA |
| | | ACAAGAAGAACGCCUACACCAUCGCCGACAACGGCGACAUGGUGCGGGUGGACGU |
| | | GUUCUGCAAGGUGGACAAGAAGGGCAAGAACCAGUACUUCAUCGUGCCCAUCUAC |
| | | GCCUGGCAGGUGGCCGAGAACAUCCUGCCCGACAUCGACUGCAAGGGCUACCGGA |
| | | UCGACGACUCCUACACCUUCUGCUUCUCCCUGCACAAGUACGACCUGAUCGCCUU |
| | | CCAGAAGGACGAGAAGUCCAAGGUGGAGUUCGCCUACUACAUCAACUGCGACUCC |
| | | UCCAACGGCCGGUUCUACCUGGCCUGGCACGACAAGGGCUCCAAGGAGCAGCAGU |
| | | UCCGGAUCUCCACCCAGAACCUGGUGCUGAUCCAGAAGUACCAGGUGAACGAGCU |
| | | GGGCAAGGAGAUCCGGCCCUGCCGGCUGAAGAAGCGGCCCCCCGUGCGGUCCGAG |
| | | UCCGCCACCCCCGAGUCCGUGUCCGGCUGGCGGCUGUUCAAGAAGAUCUCCUAGC |
| | | UAGCACCAGCCUCAAGAACACCCGAAUGGAGUCUCUAAGCUACAUAAUACCAACU |
| | | UACACUUUACAAAAUGUUGUCCCCCAAAAUGUAGCCAUUCGUAUCUGCUCCUAAU |
| | | AAAAAGAAAGUUUCUUCACAUUCUCUCGAGAAAAAAAAAAAAUGGAAAAAAAAAA |
| | | AACGGAAAAAAAAAAAAGGUAAAAAAAAAAAAUAUAAAAAAAAAAAACAUAAAAA |
| | | AAAAAAACGAAAAAAAAAAAACGUAAAAAAAAAAAACUCAAAAAAAAAAAAGAUA |
| | | AAAAAAAAAAACCUAAAAAAAAAAAAUGUAAAAAAAAAAAAGGGAAAAAAAAAAA |
| | | ACGCAAAAAAAAAAAACACAAAAAAAAAAAAUGCAAAAAAAAAAAAUCGAAAAAA |
| | | AAAAAAUCUAAAAAAAAAAAACGAAAAAAAAAAAACCCAAAAAAAAAAAAGACAA |
| | | AAAAAAAAAAUAGAAAAAAAAAAAAGUUAAAAAAAAAAAACUGAAAAAAAAAAAA |
| | | UUUAAAAAAAAAAAAUCUAG |
| 635 | mRNA Q encoding | GGGAAGCUCAGAAUAAACGCUCAACUUUGGCCGGAUCUGCCACCAUGGACGGCUC |
| | Nme2Cas 9 | CGGCGGCGGCUCCCCCAAGAAGAAGCGGAAGGUGGGCGGCUCCGGCGGCGGCGCC |
| | | GCCUUCAAGCCCAACCCCAUCAACUACAUCCUGGGCCUGGACAUCGGCAUCGCCU |
| | | CCGUGGGCUGGGCCAUGGUGGAGAUCGACGAGGAGGAGAACCCCAUCCGGCUGAU |
| | | CGACCUGGGCGUGCGGGUGUUCGAGCGGGCCGAGGUGCCCAAGACCGGCGACUCC |
| | | CUGGCCAUGGCCCGGCGGCUGGCCCGGUCCGUGCGGCGGCUGACCCGGCGGCGGG |
| | | CCCACCGGCUGCUGCGGGCCCGGCGGCUGCUGAAGCGGGAGGGCGUGCUGCAGGC |
| | | CGCCGACUUCGACGAGAACGGCCUGAUCAAGUCCCUGCCCAACACCCCCUGGCAG |
| | | CUGCGGGCCGCCGCCCUGGACCGGAAGCUGACCCCCCUGGAGUGGUCCGCCGUGC |
| | | UGCUGCACCUGAUCAAGCACCGGGGCUACCUGUCCCAGCGGAAGAACGAGGGCGA |
| | | GACCGCCGACAAGGAGCUGGGCGCCCUGCUGAAGGGCGUGGCCAACAACGCCCAC |
| | | GCCCUGCAGACCGGCGACUUCCGGACCCCCGCCGAGCUGGCCCUGAACAAGUUCG |
| | | AGAAGGAGUCCGGCCACAUCCGGAACCAGCGGGGCGACUACUCCCACACCUUCUC |
| | | CCGGAAGGACCUGCAGGCCGAGCUGAUCCUGCUGUUCGAGAAGCAGAAGGAGUUC |
| | | GGCAACCCCCACGUGUCCGGCGGCCUGAAGGAGGGCAUCGAGACCCUGCUGAUGA |
| | | CCCAGCGGCCCGCCCUGUCCGGCGACGCCGUGCAGAAGAUGCUGGGCCACUGCAC |
| | | CUUCGAGCCCGCCGAGCCCAAGGCCGCCAAGAACACCUACACCGCCGAGCGGUUC |
| | | AUCUGGCUGACCAAGCUGAACAACCUGCGGAUCCUGGAGCAGGGCUCCGAGCGGC |
| | | CCCUGACCGACACCGAGCGGGCCACCCUGAUGGACGAGCCCUACCGGAAGUCCAA |
| | | GCUGACCUACGCCCAGGCCCGGAAGCUGCUGGGCCUGGAGGACACCGCCUUCUUC |
| | | AAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAA |
| | | GUCCCCCCUGAACCUGUCCUCCGAGCUGCAGGACGAGAUCGGCACCGCCUUCUCC |
| | | CUGUUCAAGACCGACGAGGACAUCACCGGCCGGCUGAAGGACCGGGUGCAGCCCG |
| | | AGAUCCUGGAGGCCCUGCUGAAGCACAUCUCCUUCGACAAGUUCGUGCAGAUCUC |
| | | CCUGAAGGCCCUGCGGCGGAUCGUGCCCCUGAUGGAGCAGGGCAAGCGGUACGAC |
| | | GAGGCCUGCGCCGAGAUCUACGGCGACCACUACGGCAAGAAGAACACCGAGGAGA |
| | | AGAUCUACCUGCCCCCCAUCCCCGCCGACGAGAUCCGGAACCCCGUGGUGCUGCG |
| | | GGCCCUGUCCCAGGCCCGGAAGGUGAUCAACGGCGUGGUGCGGCGGUACGGCUCC |
| | | CCCGCCCGGAUCCACAUCGAGACCGCCCGGGAGGUGGGCAAGUCCUUCAAGGACC |
| | | GGAAGGAGAUCGAGAAGCGGCAGGAGGAGAACCGGAAGGACCGGGAGAAGGCCGC |
| | | CGCCAAGUUCCGGGAGUACUUCCCCAACUUCGUGGGCGAGCCCAAGUCCAAGGAC |
| | | AUCCUGAAGCUGCGGCUGUACGAGCAGCAGCACGGCAAGUGCCUGUACUCCGGCA |
| | | AGGAGAUCAACCUGGUGCGGCUGAACGAGAAGGGCUACGUGGAGAUCGACCACGC |
| | | CCUGCCCUUCUCCCGGACCUGGGACGACUCCUUCAACAACAAGGUGCUGGUGCUG |
| | | GGCUCCGAGAACCAGAACAAGGGCAACCAGACCCCCUACGAGUACUUCAACGGCA |
| | | AGGACAACUCCCGGGAGUGGCAGGAGUUCAAGGCCCGGGUGGAGACCUCCCGGUU |
| | | CCCCCGGUCCAAGAAGCAGCGGAUCCUGCUGCAGAAGUUCGACGAGGACGGCUUC |
| | | AAGGAGUGCAACCUGAACGACACCCGGUACGUGAACCGGUUCCUGUGCCAGUUCG |
| | | UGGCCGACCACAUCCUGCUGACCGGCAAGGGCAAGCGGCGGGUGUUCGCCUCCAA |
| | | CGGCCAGAUCACCAACCUGCUGCGGGGCUUCUGGGGCCUGCGGAAGGUGCGGGCC |
| | | GAGAACGACCGGCACCACGCCCUGGACGCCGUGGUGGUGGCCUGCUCCACCGUGG |
| | | CCAUGCAGCAGAAGAUCACCCGGUUCGUGCGGUACAAGGAGAUGAACGCCUUCGA |
| | | CGGCAAGACCAUCGACAAGGAGACCGGCAAGGUGCUGCACCAGAAGACCCACUUC |
| | | CCCCAGCCCUGGGAGUUCUUCGCCCAGGAGGUGAUGAUCCGGGUGUUCGGCAAGC |
| | | CCGACGGCAAGCCCGAGUUCGAGGAGGCCGACACCCCCGAGAAGCUGCGGACCCU |
| | | GCUGGCCGAGAAGCUGUCCUCCCGGCCCGAGGCCGUGCACGAGUACGUGACCCCC |
| | | CUGUUCGUGUCCCGGGCCCCCAACCGGAAGAUGUCCGGCGCCCACAAGGACACCC |
| | | UGCGGUCCGCCAAGCGGUUCGUGAAGCACAACGAGAAGAUCUCCGUGAAGCGGGU |
| | | GUGGCUGACCGAGAUCAAGCUGGCCGACCUGGAGAACAUGGUGAACUACAAGAAC |
| | | GGCCGGGAGAUCGAGCUGUACGAGGCCCUGAAGGCCCGGCUGGAGGCCUACGGCG |
| | | GCAACGCCAAGCAGGCCUUCGACCCCAAGGACAACCCCUUCUACAAGAAGGGCGG |
| | | CCAGCUGGUGAAGGCCGUGCGGGUGGAGAAGACCCAGGAGUCCGGCGUGCUGCUG |
| | | AACAAGAAGAACGCCUACACCAUCGCCGACAACGGCGACAUGGUGCGGGUGGACG |
| | | UGUUCUGCAAGGUGGACAAGAAGGGCAAGAACCAGUACUUCAUCGUGCCCAUCUA |
| | | CGCCUGGCAGGUGGCCGAGAACAUCCUGCCCGACAUCGACUGCAAGGGCUACCGG |
| | | AUCGACGACUCCUACACCUUCUGCUUCUCCCUGCACAAGUACGACCUGAUCGCCU |
| | | UCCAGAAGGACGAGAAGUCCAAGGUGGAGUUCGCCUACUACAUCAACUGCGACUC |
| | | CUCCAACGGCCGGUUCUACCUGGCCUGGCACGACAAGGGCUCCAAGGAGCAGCAG |
| | | UUCCGGAUCUCCACCCAGAACCUGGUGCUGAUCCAGAAGUACCAGGUGAACGAGC |
| | | UGGGCAAGGAGAUCCGGCCCUGCCGGCUGAAGAAGCGGCCCCCCGUGCGGUAGCU |
| | | AGCACCAGCCUCAAGAACACCCGAAUGGAGUCUCUAAGCUACAUAAUACCAACUU |
| | | ACACUUUACAAAAUGUUGUCCCCCAAAAUGUAGCCAUUCGUAUCUGCUCCUAAUA |
| | | AAAAGAAAGUUUCUUCACAUUCUCUCGAGAAAAAAAAAAAAUGGAAAAAAAAAAA |
| | | ACGGAAAAAAAAAAAAGGUAAAAAAAAAAAAUAUAAAAAAAAAAAACAUAAAAAA |
| | | AAAAAACGAAAAAAAAAAAACGUAAAAAAAAAAAACUCAAAAAAAAAAAGAUAAA |
| | | AAAAAAAAACCUAAAAAAAAAAAAUGUAAAAAAAAAAAAGGGAAAAAAAAAAAAC |
| | | GCAAAAAAAAAAAACACAAAAAAAAAAAAUGCAAAAAAAAAAAAUCGAAAAAAAA |
| | | AAAAUCUAAAAAAAAAAAACGAAAAAAAAAAAACCCAAAAAAAAAAAAGACAAAA |
| | | AAAAAAAAUAGAAAAAAAAAAAGUUAAAAAAAAAAAACUGAAAAAAAAAAAAUUU |
| | | AAAAAAAAAAAAUCUAG |
| 636 | mRNA R encoding | GGGaagctcagaataaacgctcaactttggccggatctgccaccATGGACGGCTC |
| | Nme2Cas9 base | CGGCGGCGGCTCCCCCAAGAAGAAGCGGAAGGTGGAGGACAAGCGGCCCGCCGCC |
| | editor | ACCAAGAAGGCCGGCCAGGCCAAGAAGAAGAAGGGCGGCTCCGGCGGCGGCGAGG |
| | | CCTCCCCCGCCTCCGGCCCCCGGCACCTGATGGACCCCCACATCTTCACCTCCAA |
| | | CTTCAACAACGGCATCGGCCGGCACAAGACCTACCTGTGCTACGAGGTGGAGCGG |
| | | CTGGACAACGGCACCTCCGTGAAGATGGACCAGCACCGGGGCTTCCTGCACAACC |
| | | AGGCCAAGAACCTGCTGTGCGGCTTCTACGGCCGGCACGCCGAGCTGCGGTTCCT |
| | | GGACCTGGTGCCCTCCCTGCAGCTGGACCCCGCCCAGATCTACCGGGTGACCTGG |
| | | TTCATCTCCTGGTCCCCCTGCTTCTCCTGGGGCTGCGCCGGCGAGGTGCGGGCCT |
| | | TCCTGCAGGAGAACACCCACGTGCGGCTGCGGATCTTCGCCGCCCGGATCTACGA |
| | | CTACGACCCCCTGTACAAGGAGGCCCTGCAGATGCTGCGGGACGCCGGCGCCCAG |
| | | GTGTCCATCATGACCTACGACGAGTTCAAGCACTGCTGGGACACCTTCGTGGACC |
| | | ACCAGGGCTGCCCCTTCCAGCCCTGGGACGGCCTGGACGAGCACTCCCAGGCCCT |
| | | GTCCGGCCGGCTGCGGGCCATCCTGCAGAACCAGGGCAACTCCGGCTCCGAGACC |
| | | CCCGGCACCTCCGAGTCCGCCACCCCCGAGTCCGCAGCGTTCAAACCAAATccca |
| | | tcaactacatcctgggcctggccatcggcatcgcctccgtgggctgggccatggt |
| | | ggagatcgacgaggaggagaaccccatccggctgatcgacctgggcgtgcgggtg |
| | | ttcgagcgggccgaggtgcccaagaccggcgactccctggccatggcccggcggc |
| | | tggcccggtccgtgcggcggctgacccggcggcgggcccaccggctgctgcgggc |
| | | ccggcggctgctgaagcgggagggcgtgctgcaggccgccgacttcgacgagaac |
| | | ggcctgatcaagtccctgcccaacaccccctggcagctgcgggccgccgccctgg |
| | | accggaagctgacccccctggagtggtccgccgtgctgctgcacctgatcaagca |
| | | ccggggctacctgtcccagcggaagaacgagggcgagaccgccgacaaggagctg |
| | | ggcgccctgctgaagggcgtggccaacaacgcccacgccctgcagaccggcgact |
| | | tccggacccccgccgagctggccctgaacaagttcgagaaggagtccggccacat |
| | | ccggaaccagcggggcgactactcccacaccttctcccggaaggacctgcaggcc |
| | | gagctgatcctgctgttcgagaagcagaaggagttcggcaacccccacgtgtccg |
| | | gcggcctgaaggagggcatcgagaccctgctgatgacccagcggcccgccctgtc |
| | | cggcgacgccgtgcagaagatgctgggccactgcaccttcgagcccgccgagccc |
| | | aaggccgccaagaacacctacaccgccgagcggttcatctggctgaccaagctga |
| | | acaacctgcggatcctggagcagggctccgagcggcccctgaccgacaccgagcg |
| | | ggccaccctgatggacgagccctaccggaagtccaagctgacctacgcccaggcc |
| | | cggaagctgctgggcctggaggacaccgccttcttcaagggcctgcggtacggca |
| | | aggacaacgccgaggcctccaccctgatggagatgaaggcctaccacgccatctc |
| | | ccgggccctggagaaggagggcctgaaggacaagaagtcccccctgaacctgtcc |
| | | tccgagctgcaggacgagatcggcaccgccttctccctgttcaagaccgacgagg |
| | | acatcaccggccggctgaaggaccgggtgcagcccgagatcctggaggccctgct |
| | | gaagcacatctccttcgacaagttcgtgcagatctccctgaaggccctgcggcgg |
| | | atcgtgcccctgatggagcagggcaagcggtacgacgaggcctgcgccgagatct |
| | | acggcgaccactacggcaagaagaacaccgaggagaagatctacctgccccccat |
| | | ccccgccgacgagatccggaaccccgtggtgctgcgggccctgtcccaggcccgg |
| | | aaggtgatcaacggcgtggtgcggcggtacggctcccccgcccggatccacatcg |
| | | agaccgcccgggaggtgggcaagtccttcaaggaccggaaggagatcgagaagcg |
| | | gcaggaggagaaccggaaggaccgggagaaggccgccgccaagttccgggagtac |
| | | ttccccaacttcgtgggcgagcccaagtccaaggacatcctgaagctgcggctgt |
| | | acgagcagcagcacggcaagtgcctgtactccggcaaggagatcaacctggtgcg |
| | | gctgaacgagaagggctacgtggagatcgaccacgccctgcccttctcccggacc |
| | | tgggacgactccttcaacaacaaggtgctggtgctgggctccgagaaccagaaca |
| | | agggcaaccagaccccctacgagtacttcaacggcaaggacaactcccgggagtg |
| | | gcaggagttcaaggcccgggtggagacctcccggttcccccggtccaagaagcag |
| | | cggatcctgctgcagaagttcgacgaggacggcttcaaggagtgcaacctgaacg |
| | | acacccggtacgtgaaccgcttcctgtgccagttcgtggccgaccacatcctgct |
| | | gaccggcaagggcaagcggcgggtgttcgcctccaacggccagatcaccaacctg |
| | | ctgcggggcttctggggcctgcggaaggtgcgggccgagaacgaccggcaccacg |
| | | ccctggacgccgtggtggtggcctgctccaccgtggccatgcagcagaagatcac |
| | | ccggttcgtgcggtacaaggagatgaacgccttcgacggcaagaccatcgacaag |
| | | gagaccggcaaggtgctgcaccagaagacccacttcccccagccctgggagttct |
| | | tcgcccaggaggtgatgatccgggtgttcggcaagcccgacggcaagcccgagtt |
| | | cgaggaggccgacacccccgagaagctgcggaccctgctggccgagaagctgtcc |
| | | tcccggcccgaggccgtgcacgagtacgtgacccccctgttcgtgtcccgggccc |
| | | ccaaccggaagatgtccggcgcccacaaggacaccctgcggtccgccaagcggtt |
| | | cgtgaagcacaacgagaagatctccgtgaagcgggtgtggctgaccgagatcaag |
| | | ctggccgacctggagaacatggtgaactacaagaacggccgggagatcgagctgt |
| | | acgaggccctgaaggcccggctggaggcctacggcggcaacgccaagcaggcctt |
| | | cgaccccaaggacaaccccttctacaagaagggcggccagctggtgaaggccgtg |
| | | cgggtggagaagacccaggagtccggcgtgctgctgaacaagaagaacgcctaca |
| | | ccatcgccgacaacggcgacatggtgcgggtggacgtgttctgcaaggtggacaa |
| | | gaagggcaagaaccagtacttcatcgtgcccatctacgcctggcaggtggccgag |
| | | aacatcctgcccgacatcgactgcaagggctaccggatcgacgactcctacacct |
| | | tctgcttctccctgcacaagtacgacctgatcgccttccagaaggacgagaagtc |
| | | caaggtggagttcgcctactacatcaactgcgactcctccaacggccggttctac |
| | | ctggcctggcacgacaagggctccaaggagcagcagttccggatctccacccaga |
| | | acctggtgctgatccagaagtaccaggtgaacgagctgggcaaggagatccggcc |
| | | ctgccggctgaagaagcggccccccgtgcggtccggaaagcggaccgccgacggc |
| | | tccgagttcgagtcccccaagaagaagcggaaggtggagtagTGActagcaccag |
| | | cctcaagaacacccgaatggagtctctaagctacataataccaacttacacttta |
| | | caaaatgttgtcccccaaaatgtagccattcgtatctgctcctaataaaaagaaa |
| | | gtttcttcacattctCTCGAGAAAAAAAAAAAATGGAAAAAAAAAAAACGGAAAA |
| | | AAAAAAAGGTAAAAAAAAAAAATATAAAAAAAAAAACATAAAAAAAAAAAACGAA |
| | | AAAAAAAAAACGTAAAAAAAAAAAACTCAAAAAAAAAAAGATAAAAAAAAAAAAC |
| | | CTAAAAAAAAAAAATGTAAAAAAAAAAAAGGGAAAAAAAAAAACGCAAAAAAAAA |
| | | AAACACAAAAAAAAAAAATGCAAAAAAAAAAAATCGAAAAAAAAAAAATCTAAAA |
| | | AAAAAAAACGAAAAAAAAAAAACCCAAAAAAAAAAAAGACAAAAAAAAAAAATAG |
| | | AAAAAAAAAAAGTTAAAAAAAAAAAACTGAAAAAAAAAAAATTTAAAAAAAAAAA |
| | | AT |
| 637 | mRNA S encoding | GGGAAGCUCAGAAUAAACGCUCAACUUUGGCCGGAUCUGCCACCAUGGACGGCUC |
| | Nme2Cas9 base | CGGCGGCGGCUCCCCCAAGAAGAAGCGGAAGGUGGAGGACAAGCGGCCCGCCGCC |
| | editor | ACCAAGAAGGCCGGCCAGGCCAAGAAGAAGAAGGGCGGCUCCGGCGGCGGCGAGG |
| | | CCUCCCCCGCCUCCGGCCCCCGGCACCUGAUGGACCCCCACAUCUUCACCUCCAA |
| | | CUUCAACAACGGCAUCGGCCGGCACAAGACCUACCUGUGCUACGAGGUGGAGCGG |
| | | CUGGACAACGGCACCUCCGUGAAGAUGGACCAGCACCGGGGCUUCCUGCACAACC |
| | | AGGCCAAGAACCUGCUGUGCGGCUUCUACGGCCGGCACGCCGAGCUGCGGUUCCU |
| | | GGACCUGGUGCCCUCCCUGCAGCUGGACCCCGCCCAGAUCUACCGGGUGACCUGG |
| | | UUCAUCUCCUGGUCCCCCUGCUUCUCCUGGGGCUGCGCCGGCGAGGUGCGGGCCU |
| | | UCCUGCAGGAGAACACCCACGUGCGGCUGCGGAUCUUCGCCGCCCGGAUCUACGA |
| | | CUACGACCCCCUGUACAAGGAGGCCCUGCAGAUGCUGCGGGACGCCGGCGCCCAG |
| | | GUGUCCAUCAUGACCUACGACGAGUUCAAGCACUGCUGGGACACCUUCGUGGACC |
| | | ACCAGGGCUGCCCCUUCCAGCCCUGGGACGGCCUGGACGAGCACUCCCAGGCCCU |
| | | GUCCGGCCGGCUGCGGGCCAUCCUGCAGAACCAGGGCAACUCCGGCUCCGAGACC |
| | | CCCGGCACCUCCGAGUCCGCCACCCCCGAGUCCGCAGCGUUCAAACCAAAUCCCA |
| | | UCAACUACAUCCUGGGCCUGGCCAUCGGCAUCGCCUCCGUGGGCUGGGCCAUGGU |
| | | GGAGAUCGACGAGGAGGAGAACCCCAUCCGGCUGAUCGACCUGGGCGUGCGGGUG |
| | | UUCGAGCGGGCCGAGGUGCCCAAGACCGGCGACUCCCUGGCCAUGGCCCGGCGGC |
| | | UGGCCCGGUCCGUGCGGCGGCUGACCCGGCGGCGGGCCCACCGGCUGCUGCGGGC |
| | | CCGGCGGCUGCUGAAGCGGGAGGGCGUGCUGCAGGCCGCCGACUUCGACGAGAAC |
| | | GGCCUGAUCAAGUCCCUGCCCAACACCCCCUGGCAGCUGCGGGCCGCCGCCCUGG |
| | | ACCGGAAGCUGACCCCCCUGGAGUGGUCCGCCGUGCUGCUGCACCUGAUCAAGCA |
| | | CCGGGGCUACCUGUCCCAGCGGAAGAACGAGGGCGAGACCGCCGACAAGGAGCUG |
| | | GGCGCCCUGCUGAAGGGCGUGGCCAACAACGCCCACGCCCUGCAGACCGGCGACU |
| | | UCCGGACCCCCGCCGAGCUGGCCCUGAACAAGUUCGAGAAGGAGUCCGGCCACAU |
| | | CCGGAACCAGCGGGGCGACUACUCCCACACCUUCUCCCGGAAGGACCUGCAGGCC |
| | | GAGCUGAUCCUGCUGUUCGAGAAGCAGAAGGAGUUCGGCAACCCCCACGUGUCCG |
| | | GCGGCCUGAAGGAGGGCAUCGAGACCCUGCUGAUGACCCAGCGGCCCGCCCUGUC |
| | | CGGCGACGCCGUGCAGAAGAUGCUGGGCCACUGCACCUUCGAGCCCGCCGAGCCC |
| | | AAGGCCGCCAAGAACACCUACACCGCCGAGCGGUUCAUCUGGCUGACCAAGCUGA |
| | | ACAACCUGCGGAUCCUGGAGCAGGGCUCCGAGCGGCCCCUGACCGACACCGAGCG |
| | | GGCCACCCUGAUGGACGAGCCCUACCGGAAGUCCAAGCUGACCUACGCCCAGGCC |
| | | CGGAAGCUGCUGGGCCUGGAGGACACCGCCUUCUUCAAGGGCCUGCGGUACGGCA |
| | | AGGACAACGCCGAGGCCUCCACCCUGAUGGAGAUGAAGGCCUACCACGCCAUCUC |
| | | CCGGGCCCUGGAGAAGGAGGGCCUGAAGGACAAGAAGUCCCCCCUGAACCUGUCC |
| | | UCCGAGCUGCAGGACGAGAUCGGCACCGCCUUCUCCCUGUUCAAGACCGACGAGG |
| | | ACAUCACCGGCCGGCUGAAGGACCGGGUGCAGCCCGAGAUCCUGGAGGCCCUGCU |
| | | GAAGCACAUCUCCUUCGACAAGUUCGUGCAGAUCUCCCUGAAGGCCCUGCGGCGG |
| | | AUCGUGCCCCUGAUGGAGCAGGGCAAGCGGUACGACGAGGCCUGCGCCGAGAUCU |
| | | ACGGCGACCACUACGGCAAGAAGAACACCGAGGAGAAGAUCUACCUGCCCCCCAU |
| | | CCCCGCCGACGAGAUCCGGAACCCCGUGGUGCUGCGGGCCCUGUCCCAGGCCCGG |
| | | AAGGUGAUCAACGGCGUGGUGCGGCGGUACGGCUCCCCCGCCCGGAUCCACAUCG |
| | | AGACCGCCCGGGAGGUGGGCAAGUCCUUCAAGGACCGGAAGGAGAUCGAGAAGCG |
| | | GCAGGAGGAGAACCGGAAGGACCGGGAGAAGGCCGCCGCCAAGUUCCGGGAGUAC |
| | | UUCCCCAACUUCGUGGGCGAGCCCAAGUCCAAGGACAUCCUGAAGCUGCGGCUGU |
| | | ACGAGCAGCAGCACGGCAAGUGCCUGUACUCCGGCAAGGAGAUCAACCUGGUGCG |
| | | GCUGAACGAGAAGGGCUACGUGGAGAUCGACCACGCCCUGCCCUUCUCCCGGACC |
| | | UGGGACGACUCCUUCAACAACAAGGUGCUGGUGCUGGGCUCCGAGAACCAGAACA |
| | | AGGGCAACCAGACCCCCUACGAGUACUUCAACGGCAAGGACAACUCCCGGGAGUG |
| | | GCAGGAGUUCAAGGCCCGGGUGGAGACCUCCCGGUUCCCCCGGUCCAAGAAGCAG |
| | | CGGAUCCUGCUGCAGAAGUUCGACGAGGACGGCUUCAAGGAGUGCAACCUGAACG |
| | | ACACCCGGUACGUGAACCGCUUCCUGUGCCAGUUCGUGGCCGACCACAUCCUGCU |
| | | GACCGGCAAGGGCAAGCGGCGGGUGUUCGCCUCCAACGGCCAGAUCACCAACCUG |
| | | CUGCGGGGCUUCUGGGGCCUGCGGAAGGUGCGGGCCGAGAACGACCGGCACCACG |
| | | CCCUGGACGCCGUGGUGGUGGCCUGCUCCACCGUGGCCAUGCAGCAGAAGAUCAC |
| | | CCGGUUCGUGCGGUACAAGGAGAUGAACGCCUUCGACGGCAAGACCAUCGACAAG |
| | | GAGACCGGCAAGGUGCUGCACCAGAAGACCCACUUCCCCCAGCCCUGGGAGUUCU |
| | | UCGCCCAGGAGGUGAUGAUCCGGGUGUUCGGCAAGCCCGACGGCAAGCCCGAGUU |
| | | CGAGGAGGCCGACACCCCCGAGAAGCUGCGGACCCUGCUGGCCGAGAAGCUGUCC |
| | | UCCCGGCCCGAGGCCGUGCACGAGUACGUGACCCCCCUGUUCGUGUCCCGGGCCC |
| | | CCAACCGGAAGAUGUCCGGCGCCCACAAGGACACCCUGCGGUCCGCCAAGCGGUU |
| | | CGUGAAGCACAACGAGAAGAUCUCCGUGAAGCGGGUGUGGCUGACCGAGAUCAAG |
| | | CUGGCCGACCUGGAGAACAUGGUGAACUACAAGAACGGCCGGGAGAUCGAGCUGU |
| | | ACGAGGCCCUGAAGGCCCGGCUGGAGGCCUACGGCGGCAACGCCAAGCAGGCCUU |
| | | CGACCCCAAGGACAACCCCUUCUACAAGAAGGGCGGCCAGCUGGUGAAGGCCGUG |
| | | CGGGUGGAGAAGACCCAGGAGUCCGGCGUGCUGCUGAACAAGAAGAACGCCUACA |
| | | CCAUCGCCGACAACGGCGACAUGGUGCGGGUGGACGUGUUCUGCAAGGUGGACAA |
| | | GAAGGGCAAGAACCAGUACUUCAUCGUGCCCAUCUACGCCUGGCAGGUGGCCGAG |
| | | AACAUCCUGCCCGACAUCGACUGCAAGGGCUACCGGAUCGACGACUCCUACACCU |
| | | UCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUAC |
| | | CUGGCCUGGCACGACAAGGGCUCCAAGGAGCAGCAGUUCCGGAUCUCCACCCAGA |
| | | ACCUGGUGCUGAUCCAGAAGUACCAGGUGAACGAGCUGGGCAAGGAGAUCCGGCC |
| | | CUGCCGGCUGAAGAAGCGGCCCCCCGUGCGGUAGUGACUAGCACCAGCCUCAAGA |
| | | ACACCCGAAUGGAGUCUCUAAGCUACAUAAUACCAACUUACACUUUACAAAAUGU |
| | | UGUCCCCCAAAAUGUAGCCAUUCGUAUCUGCUCCUAAUAAAAAGAAAGUUUCUUC |
| | | ACAUUCUCUCGAGAAAAAAAAAAAAUGGAAAAAAAAAAAACGGAAAAAAAAAAAG |
| | | GUAAAAAAAAAAAAUAUAAAAAAAAAAACAUAAAAAAAAAAAACGAAAAAAAAAA |
| | | AACGUAAAAAAAAAAAACUCAAAAAAAAAAAGAUAAAAAAAAAAAACCUAAAAAA |
| | | AAAAAAUGUAAAAAAAAAAAAGGGAAAAAAAAAAACGCAAAAAAAAAAAACACAA |
| | | AAAAAAAAAAUGCAAAAAAAAAAAAUCGAAAAAAAAAAAAUCUAAAAAAAAAAAA |
| | | CGAAAAAAAAAAAACCCAAAAAAAAAAAAGACAAAAAAAAAAAAUAGAAAAAAAA |
| | | AAAGUUAAAAAAAAAAAACUGAAAAAAAAAAAAUUUAAAAAAAAAAAAUCUAG |
| 638 | mRNA T encoding | GGGaagctcagaataaacgctcaactttggccggatctgccaccATGGAGGCCTC |
| | Nme2Cas9 base | CCCCGCCTCCGGCCCCCGGCACCTGATGGACCCCCACATCTTCACCTCCAACTTC |
| | editor | AACAACGGCATCGGCCGGCACAAGACCTACCTGTGCTACGAGGTGGAGCGGCTGG |
| | | ACAACGGCACCTCCGTGAAGATGGACCAGCACCGGGGCTTCCTGCACAACCAGGC |
| | | CAAGAACCTGCTGTGCGGCTTCTACGGCCGGCACGCCGAGCTGCGGTTCCTGGAC |
| | | CTGGTGCCCTCCCTGCAGCTGGACCCCGCCCAGATCTACCGGGTGACCTGGTTCA |
| | | TCTCCTGGTCCCCCTGCTTCTCCTGGGGCTGCGCCGGCGAGGTGCGGGCCTTCCT |
| | | GCAGGAGAACACCCACGTGCGGCTGCGGATCTTCGCCGCCCGGATCTACGACTAC |
| | | GACCCCCTGTACAAGGAGGCCCTGCAGATGCTGCGGGACGCCGGCGCCCAGGTGT |
| | | CCATCATGACCTACGACGAGTTCAAGCACTGCTGGGACACCTTCGTGGACCACCA |
| | | GGGCTGCCCCTTCCAGCCCTGGGACGGCCTGGACGAGCACTCCCAGGCCCTGTCC |
| | | GGCCGGCTGCGGGCCATCCTGCAGAACCAGGGCAACTCCGGCTCCGAGACCCCCG |
| | | GCACCTCCGAGTCCGCCACCCCCGAGTCCGCAGCGTTCAAACCAAATcccatcaa |
| | | ctacatcctgggcctggccatcggcatcgcctccgtgggctgggccatggtggag |
| | | atcgacgaggaggagaaccccatccggctgatcgacctgggcgtgcgggtgttcg |
| | | agcgggccgaggtgcccaagaccggcgactccctggccatggcccggcggctggc |
| | | ccggtccgtgcggcggctgacccggcggcgggcccaccggctgctgcgggcccgg |
| | | cggctgctgaagcgggagggcgtgctgcaggccgccgacttcgacgagaacggcc |
| | | tgatcaagtccctgcccaacaccccctggcagctgcgggccgccgccctggaccg |
| | | gaagctgacccccctggagtggtccgccgtgctgctgcacctgatcaagcaccgg |
| | | ggctacctgtcccagcggaagaacgagggcgagaccgccgacaaggagctgggcg |
| | | ccctgctgaagggcgtggccaacaacgcccacgccctgcagaccggcgacttccg |
| | | gacccccgccgagctggccctgaacaagttcgagaaggagtccggccacatccgg |
| | | aaccagcggggcgactactcccacaccttctcccggaaggacctgcaggccgagc |
| | | tgatcctgctgttcgagaagcagaaggagttcggcaacccccacgtgtccggcgg |
| | | cctgaaggagggcatcgagaccctgctgatgacccagcggcccgccctgtccggc |
| | | gacgccgtgcagaagatgctgggccactgcaccttcgagcccgccgagcccaagg |
| | | ccgccaagaacacctacaccgccgagcggttcatctggctgaccaagctgaacaa |
| | | cctgcggatcctggagcagggctccgagcggcccctgaccgacaccgagcgggcc |
| | | accctgatggacgagcccta |
| | | ccggaagtccaagctgacctacgcccaggcccggaagctgctgggcctggaggac |
| | | accgccttcttcaagggcctgcggtacggcaaggacaacgccgaggcctccaccc |
| | | tgatggagatgaaggcctaccacgccatctcccgggccctggagaaggagggcct |
| | | gaaggacaagaagtcccccctgaacctgtcctccgagctgcaggacgagatcggc |
| | | accgccttctccctgttcaagaccgacgaggacatcaccggccggctgaaggacc |
| | | gggtgcagcccgagatcctggaggccctgctgaagcacatctccttcgacaagtt |
| | | cgtgcagatctccctgaaggccctgcggcggatcgtgcccctgatggagcagggc |
| | | aagcggtacgacgaggcctgcgccgagatctacggcgaccactacggcaagaaga |
| | | acaccgaggagaagatctacctgccccccatccccgccgacgagatccggaaccc |
| | | cgtggtgctgcgggccctgtcccaggcccggaaggtgatcaacggcgtggtgcgg |
| | | cggtacggctcccccgcccggatccacatcgagaccgcccgggaggtgggcaagt |
| | | ccttcaaggaccggaaggagatcgagaagcggcaggaggagaaccggaaggaccg |
| | | ggagaaggccgccgccaagttccgggagtacttccccaacttcgtgggcgagccc |
| | | aagtccaaggacatcctgaagctgcggctgtacgagcagcagcacggcaagtgcc |
| | | tgtactccggcaaggagatcaacctggtgcggctgaacgagaagggctacgtgga |
| | | gatcgaccacgccctgcccttctcccggacctgggacgactccttcaacaacaag |
| | | gtgctggtgctgggctccgagaaccagaacaagggcaaccagaccccctacgagt |
| | | acttcaacggcaaggacaactcccgggagtggcaggagttcaaggcccgggtgga |
| | | gacctcccggttcccccggtccaagaagcagcggatcctgctgcagaagttcgac |
| | | gaggacggcttcaaggagtgcaacctgaacgacacccggtacgtgaaccgcttcc |
| | | tgtgccagttcgtggccgaccacatcctgctgaccggcaagggcaagcggcgggt |
| | | gttcgcctccaacggccagatcaccaacctgctgcggggcttctggggcctgcgg |
| | | aaggtgcgggccgagaacgaccggcaccacgccctggacgccgtggtggtggcct |
| | | gctccaccgtggccatgcagcagaagatcacccggttcgtgcggtacaaggagat |
| | | gaacgccttcgacggcaagaccatcgacaaggagaccggcaaggtgctgcaccag |
| | | aagacccacttcccccagccctgggagttcttcgcccaggaggtgatgatccggg |
| | | tgttcggcaagcccgacggcaagcccgagttcgaggaggccgacacccccgagaa |
| | | gctgcggaccctgctggccgagaagctgtcctcccggcccgaggccgtgcacgag |
| | | tacgtgacccccctgttcgtgtcccgggcccccaaccggaagatgtccggcgccc |
| | | acaaggacaccctgcggtccgccaagcggttcgtgaagcacaacgagaagatctc |
| | | cgtgaagcgggtgtggctgaccgagatcaagctggccgacctggagaacatggtg |
| | | aactacaagaacggccgggagatcgagctgtacgaggccctgaaggcccggctgg |
| | | aggcctacggcggcaacgccaagcaggccttcgaccccaaggacaaccccttcta |
| | | caagaagggcggccagctggtgaaggccgtgcgggtggagaagacccaggagtcc |
| | | ggcgtgctgctgaacaagaagaacgcctacaccatcgccgacaacggcgacatgg |
| | | tgcgggtggacgtgttctgcaaggtggacaagaagggcaagaaccagtacttcat |
| | | cgtgcccatctacgcctggcaggtggccgagaacatcctgcccgacatcgactgc |
| | | aagggctaccggatcgacgactcctacaccttctgcttctccctgcacaagtacg |
| | | acctgatcgccttccagaaggacgagaagtccaaggtggagttcgcctactacat |
| | | caactgcgactcctccaacggccggttctacctggcctggcacgacaagggctcc |
| | | aaggagcagcagttccggatctccacccagaacctggtgctgatccagaagtacc |
| | | aggtgaacgagctgggcaaggagatccggccctgccggctgaagaagcggccccc |
| | | cgtgcggtccggaaagcggaccgccgacggctccgagttcgagtcccccaagaag |
| | | aagcggaaggtggagtagTGActagcaccagcctcaagaacacccgaatggagtc |
| | | tctaagctacataataccaacttacactttacaaaatgttgtcccccaaaatgta |
| | | gccattcgtatctgctcctaataaaaagaaagtttcttcacattctcTCGAGAAA |
| | | AAAAAAAAATGGAAAAAAAAAAAACGGAAAAAAAAAAAGGTAAAAAAAAAAAATA |
| | | TAAAAAAAAAAACATAAAAAAAAAAAACGAAAAAAAAAAAACGTAAAAAAAAAAA |
| | | ACTCAAAAAAAAAAAGATAAAAAAAAAAAACCTAAAAAAAAAAAATGTAAAAAAA |
| | | AAAAAGGGAAAAAAAAAAACGCAAAAAAAAAAAACACAAAAAAAAAAAATGCAAA |
| | | AAAAAAAAATCGAAAAAAAAAAAATCTAAAAAAAAAAAACGAAAAAAAAAAAACC |
| | | CAAAAAAAAAAAAGACAAAAAAAAAAAATAGAAAAAAAAAAAGTTAAAAAAAAAA |
| | | AACTGAAAAAAAAAAAATTTAAAAAAAAAAAAT |
| 639 | mRNA U encoding | GGGaagctcagaataaacgctcaactttggccggatctgccacCATGaagCTGgg |
| | Nme2Cas 9 | cTCCatcGAGttcATCaagGTGaacAAGggcTCCggcTCCggcTCCGGCgccCCC |
| | | gagTCCgccACCgagTCCggcGGCaccTCCaccGAGtccGAGggcTCCgccGGCa |
| | | ccTCCaccGAGtccGAGggctccGCCggcTCCgccGGCtccaccTCCaccGAGga |
| | | gGGCaccTCCaccGAGtccGAGggctccGCCggcACCtccACCgagtccgagGGC |
| | | tccGCCggcACCtccGAGtccgccACCgagTCCggcGGCaccTCCaccGAGtccG |
| | | AGggcTCCtccTCCaccggtgccgccttcaagcccaaccccatcaactacatcct |
| | | gggcctggacatcggcatcgcctccgtgggctgggccatggtggagatcgacgag |
| | | gaggagaaccccatccggctgatcgacctgggcgtgcgggtgttcgagcgggccg |
| | | aggtgcccaagaccggcgactccctggccatggcccggcggctggcccggtccgt |
| | | gcggcggctgacccggcggcgggcccaccggctgctgcgggcccggcggctgctg |
| | | aagcgggagggcgtgctgcaggccgccgacttcgacgagaacggcctgatcaagt |
| | | ccctgcccaacaccccctggcagctgcgggccgccgccctggaccggaagctgac |
| | | ccccctggagtggtccgccgtgctgctgcacctgatcaagcaccggggctacctg |
| | | tcccagcggaagaacgagggcgagaccgccgacaaggagctgggcgccctgctga |
| | | agggcgtggccaacaacgcccacgccctgcagaccggcgacttccggacccccgc |
| | | cgagctggccctgaacaagttcgagaaggagtccggccacatccggaaccagcgg |
| | | ggcgactactcccacaccttctcccggaaggacctgcaggccgagctgatcctgc |
| | | tgttcgagaagcagaaggagttcggcaacccccacgtgtccggcggcctgaagga |
| | | gggcatcgagaccctgctgatgacccagcggcccgccctgtccggcgacgccgtg |
| | | cagaagatgctgggccactgcaccttcgagcccgccgagcccaaggccgccaaga |
| | | acacctacaccgccgagcggttcatctggctgaccaagctgaacaacctgcggat |
| | | cctggagcagggctccgagcggcccctgaccgacaccgagcgggccaccctgatg |
| | | gacgagccctaccggaagtccaagctgacctacgcccaggcccggaagctgctgg |
| | | gcctggaggacaccgccttcttcaagggcctgcggtacggcaaggacaacgccga |
| | | ggcctccaccctgatggagatgaaggcctaccacgccatctcccgggccctggag |
| | | aaggagggcctgaaggacaagaagtcccccctgaacctgtcctccgagctgcagg |
| | | acgagatcggcaccgccttctccctgttcaagaccgacgaggacatcaccggccg |
| | | gctgaaggaccgggtgcagcccgagatcctggaggccctgctgaagcacatctcc |
| | | ttcgacaagttcgtgcagatctccctgaaggccctgcggcggatcgtgcccctga |
| | | tggagcagggcaagcggtacgacgaggcctgcgccgagatctacggcgaccacta |
| | | cggcaagaagaacaccgaggagaagatctacctgccccccatccccgccgacgag |
| | | atccggaaccccgtggtgctgcgggccctgtcccaggcccggaaggtgatcaacg |
| | | gcgtggtgcggcggtacggctcccccgcccggatccacatcgagaccgcccggga |
| | | ggtgggcaagtccttcaaggaccggaaggagatcgagaagcggcaggaggagaac |
| | | cggaaggaccgggagaaggccgccgccaagttccgggagtacttccccaacttcg |
| | | tgggcgagcccaagtccaaggacatcctgaagctgcggctgtacgagcagcagca |
| | | cggcaagtgcctgtactccggcaaggagatcaacctggtgcggctgaacgagaag |
| | | ggctacgtggagatcgaccacgccctgcccttctcccggacctgggacgactcct |
| | | tcaacaacaaggtgctggtgctgggctccgagaaccagaacaagggcaaccagac |
| | | cccctacgagtacttcaacggcaaggacaactcccgggagtggcaggagttcaag |
| | | gcccgggtggagacctcccggttcccccggtccaagaagcagcggatcctgctgc |
| | | agaagttcgacgaggacggcttcaaggagtgcaacctgaacgacacccggtacgt |
| | | gaaccgcttcctgtgccagttcgtggccgaccacatcctgctgaccggcaagggc |
| | | aagcggcgggtgttcgcctccaacggccagatcaccaacctgctgcggggcttct |
| | | ggggcctgcggaaggtgcgggccgagaacgaccggcaccacgccctggacgccgt |
| | | ggtggtggcctgctccaccgtggccatgcagcagaagatcacccggttcgtgcgg |
| | | tacaaggagatgaacgccttcgacggcaagaccatcgacaaggagaccggcaagg |
| | | tgctgcaccagaagacccacttcccccagccctgggagttcttcgcccaggaggt |
| | | gatgatccgggtgttcggcaagcccgacggcaagcccgagttcgaggaggccgac |
| | | acccccgagaagctgcggaccctgctggccgagaagctgtcctcccggcccgagg |
| | | ccgtgcacgagtacgtgacccccctgttcgtgtcccgggcccccaaccggaagat |
| | | gtccggcgcccacaaggacaccctgcggtccgccaagcggttcgtgaagcacaac |
| | | gagaagatctccgtgaagcgggtgtggctgaccgagatcaagctggccgacctgg |
| | | agaacatggtgaactacaagaacggccgggagatcgagctgtacgaggccctgaa |
| | | ggcccggctggaggcctacggcggcaacgccaagcaggccttcgaccccaaggac |
| | | aaccccttctacaagaagggcggccagctggtgaaggccgtgcgggtggagaaga |
| | | cccaggagtccggcgtgctgctgaacaagaagaacgcctacaccatcgccgacaa |
| | | cggcgacatggtgcgggtggacgtgttctgcaaggtggacaagaagggcaagaac |
| | | cagtacttcatcgtgcccatctacgcctggcaggtggccgagaacatcctgcccg |
| | | acatcgactgcaagggctaccggatcgacgactcctacaccttctgcttctccct |
| | | gcacaagtacgacctgatcgccttccagaaggacgagaagtccaaggtggagttc |
| | | gcctactacatcaactgcgactcctccaacggccggttctacctggcctggcacg |
| | | acaagggctccaaggagcagcagttccggatctccacccagaacctggtgctgat |
| | | ccagaagtaccaggtgaacgagctgggcaaggagatccggccctgccggctgaag |
| | | aagcggccccccgtgcggtccggaaagcggaccgccgacggctccgagttcgagt |
| | | cccccaagaagaagcggaaggtggagtgactagcaccagcctcaagaacacccga |
| | | atggagtctctaagctacataataccaacttacactttacaaaatgttgtccccc |
| | | aaaatgtagccattcgtatctgctcctaataaaaagaaagtttcttcacattctc |
| | | TCGAGAAAAAAAAAAAATGGAAAAAAAAAAAACGGAAAAAAAAAAAAGGTAAAAA |
| | | AAAAAAATATAAAAAAAAAAAACATAAAAAAAAAAAACGAAAAAAAAAAAACGTA |
| | | AAAAAAAAAAACTCAAAAAAAAAAAAGATAAAAAAAAAAAACCTAAAAAAAAAAA |
| | | ATGTAAAAAAAAAAAAGGGAAA |
| 640 | Open reading | atgaccggtgccgccttcaagcccaaccccatcaactacatcctgggcctggaca |
| | frame for | tcggcatcgcctccgtgggctgggccatggtggagatcgacgaggaggagaaccc |
| | Nme2Cas9 | catccggctgatcgacctgggcgtgcgggtgttcgagcgggccgaggtgcccaag |
| | encoded | accggcgactccctggccatggcccggcggctggcccggtccgtgcggcggctga |
| | by mRNA A | cccggcggcgggcccaccggctgctgcgggcccggcggctgctgaagcgggaggg |
| | | cgtgctgcaggccgccgacttcgacgagaacggcctgatcaagtccctgcccaac |
| | | accccctggcagctgcgggccgccgccctggaccggaagctgacccccctggagt |
| | | ggtccgccgtgctgctgcacctgatcaagcaccggggctacctgtcccagcggaa |
| | | gaacgagggcgagaccgccgacaaggagctgggcgccctgctgaagggcgtggcc |
| | | aacaacgcccacgccctgcagaccggcgacttccggacccccgccgagctggccc |
| | | tgaacaagttcgagaaggagtccggccacatccggaaccagcggggcgactactc |
| | | ccacaccttctcccggaaggacctgcaggccgagctgatcctgctgttcgagaag |
| | | cagaaggagttcggcaacccccacgtgtccggcggcctgaaggagggcatcgaga |
| | | ccctgctgatgacccagcggcccgccctgtccggcgacgccgtgcagaagatgct |
| | | gggccactgcaccttcgagcccgccgagcccaaggccgccaagaacacctacacc |
| | | gccgagcggttcatctggctgaccaagctgaacaacctgcggatcctggagcagg |
| | | gctccgagcggcccctgaccgacaccgagcgggccaccctgatggacgagcccta |
| | | ccggaagtccaagctgacctacgcccaggcccggaagctgctgggcctggaggac |
| | | accgccttcttcaagggcctgcggtacggcaaggacaacgccgaggcctccaccc |
| | | tgatggagatgaaggcctaccacgccatctcccgggccctggagaaggagggcct |
| | | gaaggacaagaagtcccccctgaacctgtcctccgagctgcaggacgagatcggc |
| | | accgccttctccctgttcaagaccgacgaggacatcaccggccggctgaaggacc |
| | | gggtgcagcccgagatcctggaggccctgctgaagcacatctccttcgacaagtt |
| | | cgtgcagatctccctgaaggccctgcggcggatcgtgcccctgatggagcagggc |
| | | aagcggtacgacgaggcctgcgccgagatctacggcgaccactacggcaagaaga |
| | | acaccgaggagaagatctacctgccccccatccccgccgacgagatccggaaccc |
| | | cgtggtgctgcgggccctgtcccaggcccggaaggtgatcaacggcgtggtgcgg |
| | | cggtacggctcccccgcccggatccacatcgagaccgcccgggaggtgggcaagt |
| | | ccttcaaggaccggaaggagatcgagaagcggcaggaggagaaccggaaggaccg |
| | | ggagaaggccgccgccaagttccgggagtacttccccaacttcgtgggcgagccc |
| | | aagtccaaggacatcctgaagctgcggctgtacgagcagcagcacggcaagtgcc |
| | | tgtactccggcaaggagatcaacctggtgcggctgaacgagaagggctacgtgga |
| | | gatcgaccacgccctgcccttctcccggacctgggacgactccttcaacaacaag |
| | | gtgctggtgctgggctccgagaaccagaacaagggcaaccagaccccctacgagt |
| | | acttcaacggcaaggacaactcccgggagtggcaggagttcaaggcccgggtgga |
| | | gacctcccggttcccccggtccaagaagcagcggatcctgctgcagaagttcgac |
| | | gaggacggcttcaaggagtgcaacctgaacgacacccggtacgtgaaccgcttcc |
| | | tgtgccagttcgtggccgaccacatcctgctgaccggcaagggcaagcggcgggt |
| | | gttcgcctccaacggccagatcaccaacctgctgcggggcttctggggcctgcgg |
| | | aaggtgcgggccgagaacgaccggcaccacgccctggacgccgtggtggtggcct |
| | | gctccaccgtggccatgcagcagaagatcacccggttcgtgcggtacaaggagat |
| | | gaacgccttcgacggcaagaccatcgacaaggagaccggcaaggtgctgcaccag |
| | | aagacccacttcccccagccctgggagttcttcgcccaggaggtgatgatccggg |
| | | tgttcggcaagcccgacggcaagcccgagttcgaggaggccgacacccccgagaa |
| | | gctgcggaccctgctggccgagaagctgtcctcccggcccgaggccgtgcacgag |
| | | tacgtgacccccctgttcgtgtcccgggcccccaaccggaagatgtccggcgccc |
| | | acaaggacaccctgcggtccgccaagcggttcgtgaagcacaacgagaagatctc |
| | | cgtgaagcgggtgtggctgaccgagatcaagctggccgacctggagaacatggtg |
| | | aactacaagaacggccgggagatcgagctgtacgaggccctgaaggcccggctgg |
| | | aggcctacggcggcaacgccaagcaggccttcgaccccaaggacaaccccttcta |
| | | caagaagggcggccagctggtgaaggccgtgcgggtggagaagacccaggagtcc |
| | | ggcgtgctgctgaacaagaagaacgcctacaccatcgccgacaacggcgacatgg |
| | | tgcgggtggacgtgttctgcaaggtggacaagaagggcaagaaccagtacttcat |
| | | cgtgcccatctacgcctggcaggtggccgagaacatcctgcccgacatcgactgc |
| | | aagggctaccggatcgacgactcctacaccttctgcttctccctgcacaagtacg |
| | | acctgatcgccttccagaaggacgagaagtccaaggtggagttcgcctactacat |
| | | caactgcgactcctccaacggccggttctacctggcctggcacgacaagggctcc |
| | | aaggagcagcagttccggatctccacccagaacctggtgctgatccagaagtacc |
| | | aggtgaacgagctgggcaaggagatccggccctgccggctgaagaagcggccccc |
| | | cgtgcggtccggaaagcggaccgccgacggctccgagttcgagtcccccaagaag |
| | | aagcggaaggtggagtag |
| 641 | Open reading | atgaccggtgccgccttcaagcccaaccccatcaactacatcctgggcctggaca |
| | frame for | tcggcatcgcctccgtgggctgggccatggtggagatcgacgaggaggagaaccc |
| | Nme2Cas9 | catccggctgatcgacctgggcgtgcgggtgttcgagcgggccgaggtgcccaag |
| | encoded | accggcgactccctggccatggcccggcggctggcccggtccgtgcggcggctga |
| | by mRNA B | cccggcggcgggcccaccggctgctgcgggcccggcggctgctgaagcgggaggg |
| | | cgtgctgcaggccgccgacttcgacgagaacggcctgatcaagtccctgcccaac |
| | | accccctggcagctgcgggccgccgccctggaccggaagctgacccccctggagt |
| | | ggtccgccgtgctgctgcacctgatcaagcaccggggctacctgtcccagcggaa |
| | | gaacgagggcgagaccgccgacaaggagctgggcgccctgctgaagggcgtggcc |
| | | aacaacgcccacgccctgcagaccggcgacttccggacccccgccgagctggccc |
| | | tgaacaagttcgagaaggagtccggccacatccggaaccagcggggcgactactc |
| | | ccacaccttctcccggaaggacctgcaggccgagctgatcctgctgttcgagaag |
| | | cagaaggagttcggcaacccccacgtgtccggcggcctgaaggagggcatcgaga |
| | | ccctgctgatgacccagcggcccgccctgtccggcgacgccgtgcagaagatgct |
| | | gggccactgcaccttcgagcccgccgagcccaaggccgccaagaacacctacacc |
| | | gccgagcggttcatctggctgaccaagctgaacaacctgcggatcctggagcagg |
| | | gctccgagcggcccctgaccgacaccgagcgggccaccctgatggacgagcccta |
| | | ccggaagtccaagctgacctacgcccaggcccggaagctgctgggcctggaggac |
| | | accgccttcttcaagggcctgcggtacggcaaggacaacgccgaggcctccaccc |
| | | tgatggagatgaaggcctaccacgccatctcccgggccctggagaaggagggcct |
| | | gaaggacaagaagtcccccctgaacctgtcctccgagctgcaggacgagatcggc |
| | | accgccttctccctgttcaagaccgacgaggacatcaccggccggctgaaggacc |
| | | gggtgcagcccgagatcctggaggccctgctgaagcacatctccttcgacaagtt |
| | | cgtgcagatctccctgaaggccctgcggcggatcgtgcccctgatggagcagggc |
| | | aagcggtacgacgaggcctgcgccgagatctacggcgaccactacggcaagaaga |
| | | acaccgaggagaagatctacctgccccccatccccgccgacgagatccggaaccc |
| | | cgtggtgctgcgggccctgtcccaggcccggaaggtgatcaacggcgtggtgcgg |
| | | cggtacggctcccccgcccggatccacatcgagaccgcccgggaggtgggcaagt |
| | | ccttcaaggaccggaaggagatcgagaagcggcaggaggagaaccggaaggaccg |
| | | ggagaaggccgccgccaagttccgggagtacttccccaacttcgtgggcgagccc |
| | | aagtccaaggacatcctgaagctgcggctgtacgagcagcagcacggcaagtgcc |
| | | tgtactccggcaaggagatcaacctggtgcggctgaacgagaagggctacgtgga |
| | | gatcgaccacgccctgcccttctcccggacctgggacgactccttcaacaacaag |
| | | gtgctggtgctgggctccgagaaccagaacaagggcaaccagaccccctacgagt |
| | | acttcaacggcaaggacaactcccgggagtggcaggagttcaaggcccgggtgga |
| | | gacctcccggttcccccggtccaagaagcagcggatcctgctgcagaagttcgac |
| | | gaggacggcttcaaggagtgcaacctgaacgacacccggtacgtgaaccgcttcc |
| | | tgtgccagttcgtggccgaccacatcctgctgaccggcaagggcaagcggcgggt |
| | | gttcgcctccaacggccagatcaccaacctgctgcggggcttctggggcctgcgg |
| | | aaggtgcgggccgagaacgaccggcaccacgccctggacgccgtggtggtggcct |
| | | gctccaccgtggccatgcagcagaagatcacccggttcgtgcggtacaaggagat |
| | | gaacgccttcgacggcaagaccatcgacaaggagaccggcaaggtgctgcaccag |
| | | aagacccacttcccccagccctgggagttcttcgcccaggaggtgatgatccggg |
| | | tgttcggcaagcccgacggcaagcccgagttcgaggaggccgacacccccgagaa |
| | | gctgcggaccctgctggccgagaagctgtcctcccggcccgaggccgtgcacgag |
| | | tacgtgacccccctgttcgtgtcccgggcccccaaccggaagatgtccggcgccc |
| | | acaaggacaccctgcggtccgccaagcggttcgtgaagcacaacgagaagatctc |
| | | cgtgaagcgggtgtggctgaccgagatcaagctggccgacctggagaacatggtg |
| | | aactacaagaacggccgggagatcgagctgtacgaggccctgaaggcccggctgg |
| | | aggcctacggcggcaacgccaagcaggccttcgaccccaaggacaaccccttcta |
| | | caagaagggcggccagctggtgaaggccgtgcgggtggagaagacccaggagtcc |
| | | ggcgtgctgctgaacaagaagaacgcctacaccatcgccgacaacggcgacatgg |
| | | tgcgggtggacgtgttctgcaaggtggacaagaagggcaagaaccagtacttcat |
| | | cgtgcccatctacgcctggcaggtggccgagaacatcctgcccgacatcgactgc |
| | | aagggctaccggatcgacgactcctacaccttctgcttctccctgcacaagtacg |
| | | acctgatcgccttccagaaggacgagaagtccaaggtggagttcgcctactacat |
| | | caactgcgactcctccaacggccggttctacctggcctggcacgacaagggctcc |
| | | aaggagcagcagttccggatctccacccagaacctggtgctgatccagaagtacc |
| | | aggtgaacgagctgggcaaggagatccggccctgccggctgaagaagcggccccc |
| | | cgtgcggtccggaaagcggaccgccgacggctccgagttcgagtcccccaagaag |
| | | aagcggaaggtggagtag |
| 642 | Open reading | atgaccggtgccgccttcaagcccaaccccatcaactacatcctgggcctggaca |
| | frame for | tcggcatcgcctccgtgggctgggccatggtggagatcgacgaggaggagaaccc |
| | Nme2Cas9 | catccggctgatcgacctgggcgtgcgggtgttcgagcgggccgaggtgcccaag |
| | encoded | accggcgactccctggccatggcccggcggctggcccggtccgtgcggcggctga |
| | by mRNA C | cccggcggcgggcccaccggctgctgcgggcccggcggctgctgaagcgggaggg |
| | | cgtgctgcaggccgccgacttcgacgagaacggcctgatcaagtccctgcccaac |
| | | accccctggcagctgcgggccgccgccctggaccggaagctgacccccctggagt |
| | | ggtccgccgtgctgctgcacctgatcaagcaccggggctacctgtcccagcggaa |
| | | gaacgagggcgagaccgccgacaaggagctgggcgccctgctgaagggcgtggcc |
| | | aacaacgcccacgccctgcagaccggcgacttccggacccccgccgagctggccc |
| | | tgaacaagttcgagaaggagtccggccacatccggaaccagcggggcgactactc |
| | | ccacaccttctcccggaaggacctgcaggccgagctgatcctgctgttcgagaag |
| | | cagaaggagttcggcaacccccacgtgtccggcggcctgaaggagggcatcgaga |
| | | ccctgctgatgacccagcggcccgccctgtccggcgacgccgtgcagaagatgct |
| | | gggccactgcaccttcgagcccgccgagcccaaggccgccaagaacacctacacc |
| | | gccgagcggttcatctggctgaccaagctgaacaacctgcggatcctggagcagg |
| | | gctccgagcggcccctgaccgacaccgagcgggccaccctgatggacgagcccta |
| | | ccggaagtccaagctgacctacgcccaggcccggaagctgctgggcctggaggac |
| | | accgccttcttcaagggcctgcggtacggcaaggacaacgccgaggcctccaccc |
| | | tgatggagatgaaggcctaccacgccatctcccgggccctggagaaggagggcct |
| | | gaaggacaagaagtcccccctgaacctgtcctccgagctgcaggacgagatcggc |
| | | accgccttctccctgttcaagaccgacgaggacatcaccggccggctgaaggacc |
| | | gggtgcagcccgagatcctggaggccctgctgaagcacatctccttcgacaagtt |
| | | cgtgcagatctccctgaaggccctgcggcggatcgtgcccctgatggagcagggc |
| | | aagcggtacgacgaggcctgcgccgagatctacggcgaccactacggcaagaaga |
| | | acaccgaggagaagatctacctgccccccatccccgccgacgagatccggaaccc |
| | | cgtggtgctgcgggccctgtcccaggcccggaaggtgatcaacggcgtggtgcgg |
| | | cggtacggctcccccgcccggatccacatcgagaccgcccgggaggtgggcaagt |
| | | ccttcaaggaccggaaggagatcgagaagcggcaggaggagaaccggaaggaccg |
| | | ggagaaggccgccgccaagttccgggagtacttccccaacttcgtgggcgagccc |
| | | aagtccaaggacatcctgaagctgcggctgtacgagcagcagcacggcaagtgcc |
| | | tgtactccggcaaggagatcaacctggtgcggctgaacgagaagggctacgtgga |
| | | gatcgaccacgccctgcccttctcccggacctgggacgactccttcaacaacaag |
| | | gtgctggtgctgggctccgagaaccagaacaagggcaaccagaccccctacgagt |
| | | acttcaacggcaaggacaactcccgggagtggcaggagttcaaggcccgggtgga |
| | | gacctcccggttcccccggtccaagaagcagcggatcctgctgcagaagttcgac |
| | | gaggacggcttcaaggagtgcaacctgaacgacacccggtacgtgaaccgcttcc |
| | | tgtgccagttcgtggccgaccacatcctgctgaccggcaagggcaagcggcgggt |
| | | gttcgcctccaacggccagatcaccaacctgctgcggggcttctggggcctgcgg |
| | | aaggtgcgggccgagaacgaccggcaccacgccctggacgccgtggtggtggcct |
| | | gctccaccgtggccatgcagcagaagatcacccggttcgtgcggtacaaggagat |
| | | gaacgccttcgacggcaagaccatcgacaaggagaccggcaaggtgctgcaccag |
| | | aagacccacttcccccagccctgggagttcttcgcccaggaggtgatgatccggg |
| | | tgttcggcaagcccgacggcaagcccgagttcgaggaggccgacacccccgagaa |
| | | gctgcggaccctgctggccgagaagctgtcctcccggcccgaggccgtgcacgag |
| | | tacgtgacccccctgttcgtgtcccgggcccccaaccggaagatgtccggcgccc |
| | | acaaggacaccctgcggtccgccaagcggttcgtgaagcacaacgagaagatctc |
| | | cgtgaagcgggtgtggctgaccgagatcaagctggccgacctggagaacatggtg |
| | | aactacaagaacggccgggagatcgagctgtacgaggccctgaaggcccggctgg |
| | | aggcctacggcggcaacgccaagcaggccttcgaccccaaggacaaccccttcta |
| | | caagaagggcggccagctggtgaaggccgtgcgggtggagaagacccaggagtcc |
| | | ggcgtgctgctgaacaagaagaacgcctacaccatcgccgacaacggcgacatgg |
| | | tgcgggtggacgtgttctgcaaggtggacaagaagggcaagaaccagtacttcat |
| | | cgtgcccatctacgcctggcaggtggccgagaacatcctgcccgacatcgactgc |
| | | aagggctaccggatcgacgactcctacaccttctgcttctccctgcacaagtacg |
| | | acctgatcgccttccagaaggacgagaagtccaaggtggagttcgcctactacat |
| | | caactgcgactcctccaacggccggttctacctggcctggcacgacaagggctcc |
| | | aaggagcagcagttccggatctccacccagaacctggtgctgatccagaagtacc |
| | | aggtgaacgagctgggcaaggagatccggccctgccggctgaagaagcggccccc |
| | | cgtgcggtccggaaagcggaccgccgacggctccgagttcgagtcccccaagaag |
| | | aagcggaaggtggagtag |
| 643 | Open reading | atgaccggtgccgccttcaagcccaaccccatcaactacatcctgggcctggaca |
| | frame for | tcggcatcgcctccgtgggctgggccatggtggagatcgacgaggaggagaaccc |
| | Nme2Cas9 | catccggctgatcgacctgggcgtgcgggtgttcgagcgggccgaggtgcccaag |
| | encoded | accggcgactccctggccatggcccggcggctggcccggtccgtgcggcggctga |
| | by mRNA D | cccggcggcgggcccaccggctgctgcgggcccggcggctgctgaagcgggaggg |
| | | cgtgctgcaggccgccgacttcgacgagaacggcctgatcaagtccctgcccaac |
| | | accccctggcagctgcgggccgccgccctggaccggaagctgacccccctggagt |
| | | ggtccgccgtgctgctgcacctgatcaagcaccggggctacctgtcccagcggaa |
| | | gaacgagggcgagaccgccgacaaggagctgggcgccctgctgaagggcgtggcc |
| | | aacaacgcccacgccctgcagaccggcgacttccggacccccgccgagctggccc |
| | | tgaacaagttcgagaaggagtccggccacatccggaaccagcggggcgactactc |
| | | ccacaccttctcccggaaggacctgcaggccgagctgatcctgctgttcgagaag |
| | | cagaaggagttcggcaacccccacgtgtccggcggcctgaaggagggcatcgaga |
| | | ccctgctgatgacccagcggcccgccctgtccggcgacgccgtgcagaagatgct |
| | | gggccactgcaccttcgagcccgccgagcccaaggccgccaagaacacctacacc |
| | | gccgagcggttcatctggctgaccaagctgaacaacctgcggatcctggagcagg |
| | | gctccgagcggcccctgaccgacaccgagcgggccaccctgatggacgagcccta |
| | | ccggaagtccaagctgacctacgcccaggcccggaagctgctgggcctggaggac |
| | | accgccttcttcaagggcctgcggtacggcaaggacaacgccgaggcctccaccc |
| | | tgatggagatgaaggcctaccacgccatctcccgggccctggagaaggagggcct |
| | | gaaggacaagaagtcccccctgaacctgtcctccgagctgcaggacgagatcggc |
| | | accgccttctccctgttcaagaccgacgaggacatcaccggccggctgaaggacc |
| | | gggtgcagcccgagatcctggaggccctgctgaagcacatctccttcgacaagtt |
| | | cgtgcagatctccctgaaggccctgcggcggatcgtgcccctgatggagcagggc |
| | | aagcggtacgacgaggcctgcgccgagatctacggcgaccactacggcaagaaga |
| | | acaccgaggagaagatctacctgccccccatccccgccgacgagatccggaaccc |
| | | cgtggtgctgcgggccctgtcccaggcccggaaggtgatcaacggcgtggtgcgg |
| | | cggtacggctcccccgcccggatccacatcgagaccgcccgggaggtgggcaagt |
| | | ccttcaaggaccggaaggagatcgagaagcggcaggaggagaaccggaaggaccg |
| | | ggagaaggccgccgccaagttccgggagtacttccccaacttcgtgggcgagccc |
| | | aagtccaaggacatcctgaagctgcggctgtacgagcagcagcacggcaagtgcc |
| | | tgtactccggcaaggagatcaacctggtgcggctgaacgagaagggctacgtgga |
| | | gatcgaccacgccctgcccttctcccggacctgggacgactccttcaacaacaag |
| | | gtgctggtgctgggctccgagaaccagaacaagggcaaccagaccccctacgagt |
| | | acttcaacggcaaggacaactcccgggagtggcaggagttcaaggcccgggtgga |
| | | gacctcccggttcccccggtccaagaagcagcggatcctgctgcagaagttcgac |
| | | gaggacggcttcaaggagtgcaacctgaacgacacccggtacgtgaaccgcttcc |
| | | tgtgccagttcgtggccgaccacatcctgctgaccggcaagggcaagcggcgggt |
| | | gttcgcctccaacggccagatcaccaacctgctgcggggcttctggggcctgcgg |
| | | aaggtgcgggccgagaacgaccggcaccacgccctggacgccgtggtggtggcct |
| | | gctccaccgtggccatgcagcagaagatcacccggttcgtgcggtacaaggagat |
| | | gaacgccttcgacggcaagaccatcgacaaggagaccggcaaggtgctgcaccag |
| | | aagacccacttcccccagccctgggagttcttcgcccaggaggtgatgatccggg |
| | | tgttcggcaagcccgacggcaagcccgagttcgaggaggccgacacccccgagaa |
| | | gctgcggaccctgctggccgagaagctgtcctcccggcccgaggccgtgcacgag |
| | | tacgtgacccccctgttcgtgtcccgggcccccaaccggaagatgtccggcgccc |
| | | acaaggacaccctgcggtccgccaagcggttcgtgaagcacaacgagaagatctc |
| | | cgtgaagcgggtgtggctgaccgagatcaagctggccgacctggagaacatggtg |
| | | aactacaagaacggccgggagatcgagctgtacgaggccctgaaggcccggctgg |
| | | aggcctacggcggcaacgccaagcaggccttcgaccccaaggacaaccccttcta |
| | | caagaagggcggccagctggtgaaggccgtgcgggtggagaagacccaggagtcc |
| | | ggcgtgctgctgaacaagaagaacgcctacaccatcgccgacaacggcgacatgg |
| | | tgcgggtggacgtgttctgcaaggtggacaagaagggcaagaaccagtacttcat |
| | | cgtgcccatctacgcctggcaggtggccgagaacatcctgcccgacatcgactgc |
| | | aagggctaccggatcgacgactcctacaccttctgcttctccctgcacaagtacg |
| | | acctgatcgccttccagaaggacgagaagtccaaggtggagttcgcctactacat |
| | | caactgcgactcctccaacggccggttctacctggcctggcacgacaagggctcc |
| | | aaggagcagcagttccggatctccacccagaacctggtgctgatccagaagtacc |
| | | aggtgaacgagctgggcaaggagatccggccctgccggctgaagaagcggccccc |
| | | cgtgcggtccggaaagcggaccgccgacggctccgagttcgagtcccccaagaag |
| | | aagcggaaggtggagtag |
| 644 | Open reading | ATGgaggcctcccccgcctccggcccccggcacctgatggacccccacatcttca |
| | frame for | cctccAACTTCAACAACggcATCggccggCACAAGaccTACCTGTGCTACgaggt |
| | SpyCas9 base | ggagcggCTGGACAACggcacctccgtgAAGATGGACCAGCACcggggcTTCCTG |
| | editor encoded | CACAACCAGgccAAGAACCTGCTGTGCggcTTCTACggccggCACgccgagCTGc |
| | by mRNA E | ggTTCCTGGACCTGgtgccctccCTGCAGCTGGACcccgccCAGATCTACcgggt |
| | | gaccTGGTTCATCtccTGGtcccccTGCTTCtccTGGggcTGCgccggcgaggtg |
| | | cgggccTTCCTGCAGgagAACaccCACgtgcggCTGcggATCTTCgccgcccggA |
| | | TCTACGACTACGACcccCTGTACAAGgaggccCTGCAGATGCTGcggGACgccgg |
| | | cgccCAGgtgtccATCATGaccTACGACgagTTCAAGCACTGCTGGGACaccTTC |
| | | gtgGACCACCAGggcTGCCCCTTCCAGcccTGGGACggcCTGGACgagCACtccC |
| | | AGgccCTGtccggccggCTGcgggccATCCTGCAGAACCAGggcAACtccggctc |
| | | cgagacccccggcacctccgagtccgccacccccgagtccgacaagaagtactcc |
| | | atcggcctggCcatcggcaccaactccgtgggctgggccgtgatcaccgacgagt |
| | | acaaggtgccctccaagaagttcaaggtgctgggcaacaccgaccggcactccat |
| | | caagaagaacctgatcggcgccctgctgttcgactccggcgagaccgccgaggcc |
| | | acccggctgaagcggaccgcccggcggcggtacacccggcggaagaaccggatct |
| | | gctacctgcaggagatcttctccaacgagatggccaaggtggacgactccttctt |
| | | ccaccggctggaggagtccttcctggtggaggaggacaagaagcacgagcggcac |
| | | cccatcttcggcaacatcgtggacgaggtggcctaccacgagaagtaccccacca |
| | | tctaccacctgcggaagaagctggtggactccaccgacaaggccgacctgcggct |
| | | gatctacctggccctggcccacatgatcaagttccggggccacttcctgatcgag |
| | | ggcgacctgaaccccgacaactccgacgtggacaagctgttcatccagctggtgc |
| | | agacctacaaccagctgttcgaggagaaccccatcaacgcctccggcgtggacgc |
| | | caaggccatcctgtccgcccggctgtccaagtcccggcggctggagaacctgatc |
| | | gcccagctgcccggcgagaagaagaacggcctgttcggcaacctgatcgccctgt |
| | | ccctgggcctgacccccaacttcaagtccaacttcgacctggccgaggacgccaa |
| | | gctgcagctgtccaaggacacctacgacgacgacctggacaacctgctggcccag |
| | | atcggcgaccagtacgccgacctgttcctggccgccaagaacctgtccgacgcca |
| | | tcctgctgtccgacatcctgcgggtgaacaccgagatcaccaaggcccccctgtc |
| | | cgcctccatgatcaagcggtacgacgagcaccaccaggacctgaccctgctgaag |
| | | gccctggtgcggcagcagctgcccgagaagtacaaggagatcttcttcgaccagt |
| | | ccaagaacggctacgccggctacatcgacggcggcgcctcccaggaggagttcta |
| | | caagttcatcaagcccatcctggagaagatggacggcaccgaggagctgctggtg |
| | | aagctgaaccgggaggacctgctgcggaagcagcggaccttcgacaacggctcca |
| | | tcccccaccagatccacctgggcgagctgcacgccatcctgcggcggcaggagga |
| | | cttctaccccttcctgaaggacaaccgggagaagatcgagaagatcctgaccttc |
| | | cggatcccctactacgtgggccccctggcccggggcaactcccggttcgcctgga |
| | | tgacccggaagtccgaggagaccatcaccccctggaacttcgaggaggtggtgga |
| | | caagggcgcctccgcccagtccttcatcgagcggatgaccaacttcgacaagaac |
| | | ctgcccaacgagaaggtgctgcccaagcactccctgctgtacgagtacttcaccg |
| | | tgtacaacgagctgaccaaggtgaagtacgtgaccgagggcatgcggaagcccgc |
| | | cttcctgtccggcgagcagaagaaggccatcgtggacctgctgttcaagaccaac |
| | | cggaaggtgaccgtgaagcagctgaaggaggactacttcaagaagatcgagtgct |
| | | tcgactccgtggagatctccggcgtggaggaccggttcaacgcctccctgggcac |
| | | ctaccacgacctgctgaagatcatcaaggacaaggacttcctggacaacgaggag |
| | | aacgaggacatcctggaggacatcgtgctgaccctgaccctgttcgaggaccggg |
| | | agatgatcgaggagcggctgaagacctacgcccacctgttcgacgacaaggtgat |
| | | gaagcagctgaagcggcggcggtacaccggctggggccggctgtcccggaagctg |
| | | atcaacggcatccgggacaagcagtccggcaagaccatcctggacttcctgaagt |
| | | ccgacggcttcgccaaccggaacttcatgcagctgatccacgacgactccctgac |
| | | cttcaaggaggacatccagaaggcccaggtgtccggccagggcgactccctgcac |
| | | gagcacatcgccaacctggccggctcccccgccatcaagaagggcatcctgcaga |
| | | ccgtgaaggtggtggacgagctggtgaaggtgatgggccggcacaagcccgagaa |
| | | catcgtgatcgagatggcccgggagaaccagaccacccagaagggccagaagaac |
| | | tcccgggagcggatgaagcggatcgaggagggcatcaaggagctgggctcccaga |
| | | tcctgaaggagcaccccgtggagaacacccagctgcagaacgagaagctgtacct |
| | | gtactacctgcagaacggccgggacatgtacgtggaccaggagctggacatcaac |
| | | cggctgtccgactacgacgtggaccacatcgtgccccagtccttcctgaaggacg |
| | | actccatcgacaacaaggtgctgacccggtccgacaagaaccggggcaagtccga |
| | | caacgtgccctccgaggaggtggtgaagaagatgaagaactactggcggcagctg |
| | | ctgaacgccaagctgatcacccagcggaagttcgacaacctgaccaaggccgagc |
| | | ggggcggcctgtccgagctggacaaggccggcttcatcaagcggcagctggtgga |
| | | gacccggcagatcaccaagcacgtggcccagatcctggactcccggatgaacacc |
| | | aagtacgacgagaacgacaagctgatccgggaggtgaaggtgatcaccctgaagt |
| | | ccaagctggtgtccgacttccggaaggacttccagttctacaaggtgcgggagat |
| | | caacaactaccaccacgcccacgacgcctacctgaacgccgtggtgggcaccgcc |
| | | ctgatcaagaagtaccccaagctggagtccgagttcgtgtacggcgactacaagg |
| | | tgtacgacgtgcggaagatgatcgccaagtccgagcaggagatcggcaaggccac |
| | | cgccaagtacttcttctactccaacatcatgaacttcttcaagaccgagatcacc |
| | | ctggccaacggcgagatccggaagcggcccctgatcgagaccaacggcgagaccg |
| | | gcgagatcgtgtgggacaagggccgggacttcgccaccgtgcggaaggtgctgtc |
| | | catgccccaggtgaacatcgtgaagaagaccgaggtgcagaccggcggcttctcc |
| | | aaggagtccatcctgcccaagcggaactccgacaagctgatcgcccggaagaagg |
| | | actgggaccccaagaagtacggcggcttcgactcccccaccgtggcctactccgt |
| | | gctggtggtggccaaggtggagaagggcaagtccaagaagctgaagtccgtgaag |
| | | gagctgctgggcatcaccatcatggagcggtcctccttcgagaagaaccccatcg |
| | | acttcctggaggccaagggctacaaggaggtgaagaaggacctgatcatcaagct |
| | | gcccaagtactccctgttcgagctggagaacggccggaagcggatgctggcctcc |
| | | gccggcgagctgcagaagggcaacgagctggccctgccctccaagtacgtgaact |
| | | tcctgtacctggcctcccactacgagaagctgaagggctcccccgaggacaacga |
| | | gcagaagcagctgttcgtggagcagcacaagcactacctggacgagatcatcgag |
| | | cagatctccgagttctccaagcgggtgatcctggccgacgccaacctggacaagg |
| | | tgctgtccgcctacaacaagcaccgggacaagcccatccgggagcaggccgagaa |
| | | catcatccacctgttcaccctgaccaacctgggcgcccccgccgccttcaagtac |
| | | ttcgacaccaccatcgaccggaagcggtacacctccaccaaggaggtgctggacg |
| | | ccaccctgatccaccagtccatcaccggcctgtacgagacccggatcgacctgtc |
| | | ccagctgggcggcgacggcggcggctcccccaagaagaagcggaaggtgTgA |
| 645 | Open reading | ATGGCAGCATTCAAGCCGAACTCGATCAACTACATCCTGGGACTGGACATCGGAA |
| | frame for | TCGCATCGGTCGGATGGGCAATGGTCGAAATCGACGAAGAAGAAAACCCGATCAG |
| | NmelCas9 | ACTGATCGACCTGGGAGTCAGAGTCTTCGAAAGAGCAGAAGTCCCGAAGACAGGA |
| | encoded | GACTCGCTGGCAATGGCAAGAAGACTGGCAAGATCGGTCAGAAGACTGACAAGAA |
| | by mRNA F | GAAGAGCACACAGACTGCTGAGAACAAGAAGACTGCTGAAGAGAGAAGGAGTCCT |
| | | GCAGGCAGCAAACTTCGACGAAAACGGACTGATCAAGTCGCTGCCGAACACACCG |
| | | TGGCAGCTGAGAGCAGCAGCACTGGACAGAAAGCTGACACCGCTGGAATGGTCGG |
| | | CAGTCCTGCTGCACCTGATCAAGCACAGAGGATACCTGTCGCAGAGAAAGAACGA |
| | | AGGAGAAACAGCAGACAAGGAACTGGGAGCACTGCTGAAGGGAGTCGCAGGAAAC |
| | | GCACACGCACTGCAGACAGGAGACTTCAGAACACCGGCAGAACTGGCACTGAACA |
| | | AGTTCGAAAAGGAATCGGGACACATCAGAAACCAGAGATCGGACTACTCGCACAC |
| | | ATTCTCGAGAAAGGACCTGCAGGCAGAACTGATCCTGCTGTTCGAAAAGCAGAAG |
| | | GAATTCGGAAACCCGCACGTCTCGGGAGGACTGAAGGAAGGAATCGAAACACTGC |
| | | TGATGACACAGAGACCGGCACTGTCGGGAGACGCAGTCCAGAAGATGCTGGGACA |
| | | CTGCACATTCGAACCGGCAGAACCGAAGGCAGCAAAGAACACATACACAGCAGAA |
| | | AGATTCATCTGGCTGACAAAGCTGAACAACCTGAGAATCCTGGAACAGGGATCGG |
| | | AAAGACCGCTGACAGACACAGAAAGAGCAACACTGATGGACGAACCGTACAGAAA |
| | | GTCGAAGCTGACATACGCACAGGCAAGAAAGCTGCTGGGACTGGAAGACACAGCA |
| | | TTCTTCAAGGGACTGAGATACGGAAAGGACAACGCAGAAGCATCGACACTGATGG |
| | | AAATGAAGGCATACCACGCAATCTCGAGAGCACTGGAAAAGGAAGGACTGAAGGA |
| | | CAAGAAGTCGCCGCTGAACCTGTCGCCGGAACTGCAGGACGAAATCGGAACAGCA |
| | | TTCTCGCTGTTCAAGACAGACGAAGACATCACAGGAAGACTGAAGGACAGAATCC |
| | | AGCCGGAAATCCTGGAAGCACTGCTGAAGCACATCTCGTTCGACAAGTTCGTCCA |
| | | GATCTCGCTGAAGGCACTGAGAAGAATCGTCCCGCTGATGGAACAGGGAAAGAGA |
| | | TACGACGAAGCATGCGCAGAAATCTACGGAGACCACTACGGAAAGAAGAACACAG |
| | | AAGAAAAGATCTACCTGCCGCCGATCCCGGCAGACGAAATCAGAAACCCGGTCGT |
| | | CCTGAGAGCACTGTCGCAGGCAAGAAAGGTCATCAACGGAGTCGTCAGAAGATAC |
| | | GGATCGCCGGCAAGAATCCACATCGAAACAGCAAGAGAAGTCGGAAAGTCGTTCA |
| | | AGGACAGAAAGGAAATCGAAAAGAGACAGGAAGAAAACAGAAAGGACAGAGAAAA |
| | | GGCAGCAGCAAAGTTCAGAGAATACTTCCCGAACTTCGTCGGAGAACCGAAGTCG |
| | | AAGGACATCCTGAAGCTGAGACTGTACGAACAGCAGCACGGAAAGTGCCTGTACT |
| | | CGGGAAAGGAAATCAACCTGGGAAGACTGAACGAAAAGGGATACGTCGAAATCGA |
| | | CCACGCACTGCCGTTCTCGAGAACATGGGACGACTCGTTCAACAACAAGGTCCTG |
| | | GTCCTGGGATCGGAAAACCAGAACAAGGGAAACCAGACACCGTACGAATACTTCA |
| | | ACGGAAAGGACAACTCGAGAGAATGGCAGGAATTCAAGGCAAGAGTCGAAACATC |
| | | GAGATTCCCGAGATCGAAGAAGCAGAGAATCCTGCTGCAGAAGTTCGACGAAGAC |
| | | GGATTCAAGGAAAGAAACCTGAACGACACAAGATACGTCAACAGATTCCTGTGCC |
| | | AGTTCGTCGCAGACAGAATGAGACTGACAGGAAAGGGAAAGAAGAGAGTCTTCGC |
| | | ATCGAACGGACAGATCACAAACCTGCTGAGAGGATTCTGGGGACTGAGAAAGGTC |
| | | AGAGCAGAAAACGACAGACACCACGCACTGGACGCAGTCGTCGTCGCATGCTCGA |
| | | CAGTCGCAATGCAGCAGAAGATCACAAGATTCGTCAGATACAAGGAAATGAACGC |
| | | ATTCGACGGAAAGACAATCGACAAGGAAACAGGAGAAGTCCTGCACCAGAAGACA |
| | | CACTTCCCGCAGCCGTGGGAATTCTTCGCACAGGAAGTCATGATCAGAGTCTTCG |
| | | GAAAGCCGGACGGAAAGCCGGAATTCGAAGAAGCAGACACACTGGAAAAGCTGAG |
| | | AACACTGCTGGCAGAAAAGCTGTCGTCGAGACCGGAAGCAGTCCACGAATACGTC |
| | | ACACCGCTGTTCGTCTCGAGAGCACCGAACAGAAAGATGTCGGGACAGGGACACA |
| | | TGGAAACAGTCAAGTCGGCAAAGAGACTGGACGAAGGAGTCTCGGTCCTGAGAGT |
| | | CCCGCTGACACAGCTGAAGCTGAAGGACCTGGAAAAGATGGTCAACAGAGAAAGA |
| | | GAACCGAAGCTGTACGAAGCACTGAAGGCAAGACTGGAAGCACACAAGGACGACC |
| | | CGGCAAAGGCATTCGCAGAACCGTTCTACAAGTACGACAAGGCAGGAAACAGAAC |
| | | ACAGCAGGTCAAGGCAGTCAGAGTCGAACAGGTCCAGAAGACAGGAGTCTGGGTC |
| | | AGAAACCACAACGGAATCGCAGACAACGCAACAATGGTCAGAGTAGACGTCTTCG |
| | | AAAAGGGAGACAAGTACTACCTGGTCCCGATCTACTCGTGGCAGGTCGCAAAGGG |
| | | AATCCTGCCGGACAGAGCAGTCGTCCAGGGAAAGGACGAAGAAGACTGGCAGCTG |
| | | ATCGACGACTCGTTCAACTTCAAGTTCTCGCTGCACCCGAACGACCTGGTCGAAG |
| | | TCATCACAAAGAAGGCAAGAATGTTCGGATACTTCGCATCGTGCCACAGAGGAAC |
| | | AGGAAACATCAACATCAGAATCCACGACCTGGACCACAAGATCGGAAAGAACGGA |
| | | ATCCTGGAAGGAATCGGAGTCAAGACAGCACTGTCGTTCCAGAAGTACCAGATCG |
| | | ACGAACTGGGAAAGGAAATCAGACCGTGCAGACTGAAGAAGAGACCGCCGGTCAG |
| | | ATCCGGAAAGAGAACAGCAGACGGATCGGAATTCGAATCGCCGAAGAAGAAGAGA |
| | | AAGGTCGAATGA |
| 646 | Open reading | ATGACCAACCTGTCCGACATCATCGAGAAGGAGACCGGCAAGCAGCTGGTGATCC |
| | frame for UGI | AGGAGTCCATCCTGATGCTGCCCGAGGAGGTGGAGGAGGTGATCGGCAACAAGCC |
| | encoded by mRNA | CGAGTCCGACATCCTGGTGCACACCGCCTACGACGAGTCCACCGACGAGAACGTG |
| | G | ATGCTGCTGACCTCCGACGCCCCCGAGTACAAGCCCTGGGCCCTGGTGATCCAGG |
| | | ACTCCAACGGCGAGAACAAGATCAAGATGCTGTCCGGCGGCTCCAAGCGGACCGC |
| | | CGACGGCTCCGAGTTCGAGTCCCCCAAGAAGAAGCGGAAGGTGGAGTGATAG |
| 647 | Open reading | ATGGCCGCCTTCAAGCCCAACCCCATCAACTACATCCTGGGCCTGGACATCGGCA |
| | frame for | TCGCCTCCGTGGGCTGGGCCATGGTGGAGATCGACGAGGAGGAGAACCCCATCCG |
| | Nme2Cas9 | GCTGATCGACCTGGGCGTGCGGGTGTTCGAGCGGGCCGAGGTGCCCAAGACCGGC |
| | encoded | GACTCCCTGGCCATGGCCCGGCGGCTGGCCCGGTCCGTGCGGCGGCTGACCCGGC |
| | by mRNA H | GGCGGGCCCACCGGCTGCTGCGGGCCCGGCGGCTGCTGAAGCGGGAGGGCGTGCT |
| | | GCAGGCCGCCGACTTCGACGAGAACGGCCTGATCAAGTCCCTGCCCAACACCCCC |
| | | TGGCAGCTGCGGGCCGCCGCCCTGGACCGGAAGCTGACCCCCCTGGAGTGGTCCG |
| | | CCGTGCTGCTGCACCTGATCAAGCACCGGGGCTACCTGTCCCAGCGGAAGAACGA |
| | | GGGCGAGACCGCCGACAAGGAGCTGGGCGCCCTGCTGAAGGGCGTGGCCAACAAC |
| | | GCCCACGCCCTGCAGACCGGCGACTTCCGGACCCCCGCCGAGCTGGCCCTGAACA |
| | | AGTTCGAGAAGGAGTCCGGCCACATCCGGAACCAGCGGGGCGACTACTCCCACAC |
| | | CTTCTCCCGGAAGGACCTGCAGGCCGAGCTGATCCTGCTGTTCGAGAAGCAGAAG |
| | | GAGTTCGGCAACCCCCACGTGTCCGGCGGCCTGAAGGAGGGCATCGAGACCCTGC |
| | | TGATGACCCAGCGGCCCGCCCTGTCCGGCGACGCCGTGCAGAAGATGCTGGGCCA |
| | | CTGCACCTTCGAGCCCGCCGAGCCCAAGGCCGCCAAGAACACCTACACCGCCGAG |
| | | CGGTTCATCTGGCTGACCAAGCTGAACAACCTGCGGATCCTGGAGCAGGGCTCCG |
| | | AGCGGCCCCTGACCGACACCGAGCGGGCCACCCTGATGGACGAGCCCTACCGGAA |
| | | GTCCAAGCTGACCTACGCCCAGGCCCGGAAGCTGCTGGGCCTGGAGGACACCGCC |
| | | TTCTTCAAGGGCCTGCGGTACGGCAAGGACAACGCCGAGGCCTCCACCCTGATGG |
| | | AGATGAAGGCCTACCACGCCATCTCCCGGGCCCTGGAGAAGGAGGGCCTGAAGGA |
| | | CAAGAAGTCCCCCCTGAACCTGTCCTCCGAGCTGCAGGACGAGATCGGCACCGCC |
| | | TTCTCCCTGTTCAAGACCGACGAGGACATCACCGGCCGGCTGAAGGACCGGGTGC |
| | | AGCCCGAGATCCTGGAGGCCCTGCTGAAGCACATCTCCTTCGACAAGTTCGTGCA |
| | | GATCTCCCTGAAGGCCCTGCGGCGGATCGTGCCCCTGATGGAGCAGGGCAAGCGG |
| | | TACGACGAGGCCTGCGCCGAGATCTACGGCGACCACTACGGCAAGAAGAACACCG |
| | | AGGAGAAGATCTACCTGCCCCCCATCCCCGCCGACGAGATCCGGAACCCCGTGGT |
| | | GCTGCGGGCCCTGTCCCAGGCCCGGAAGGTGATCAACGGCGTGGTGCGGCGGTAC |
| | | GGCTCCCCCGCCCGGATCCACATCGAGACCGCCCGGGAGGTGGGCAAGTCCTTCA |
| | | AGGACCGGAAGGAGATCGAGAAGCGGCAGGAGGAGAACCGGAAGGACCGGGAGAA |
| | | GGCCGCCGCCAAGTTCCGGGAGTACTTCCCCAACTTCGTGGGCGAGCCCAAGTCC |
| | | AAGGACATCCTGAAGCTGCGGCTGTACGAGCAGCAGCACGGCAAGTGCCTGTACT |
| | | CCGGCAAGGAGATCAACCTGGTGCGGCTGAACGAGAAGGGCTACGTGGAGATCGA |
| | | CCACGCCCTGCCCTTCTCCCGGACCTGGGACGACTCCTTCAACAACAAGGTGCTG |
| | | GTGCTGGGCTCCGAGAACCAGAACAAGGGCAACCAGACCCCCTACGAGTACTTCA |
| | | ACGGCAAGGACAACTCCCGGGAGTGGCAGGAGTTCAAGGCCCGGGTGGAGACCTC |
| | | CCGGTTCCCCCGGTCCAAGAAGCAGCGGATCCTGCTGCAGAAGTTCGACGAGGAC |
| | | GGCTTCAAGGAGTGCAACCTGAACGACACCCGGTACGTGAACCGGTTCCTGTGCC |
| | | AGTTCGTGGCCGACCACATCCTGCTGACCGGCAAGGGCAAGCGGCGGGTGTTCGC |
| | | CTCCAACGGCCAGATCACCAACCTGCTGCGGGGCTTCTGGGGCCTGCGGAAGGTG |
| | | CGGGCCGAGAACGACCGGCACCACGCCCTGGACGCCGTGGTGGTGGCCTGCTCCA |
| | | CCGTGGCCATGCAGCAGAAGATCACCCGGTTCGTGCGGTACAAGGAGATGAACGC |
| | | CTTCGACGGCAAGACCATCGACAAGGAGACCGGCAAGGTGCTGCACCAGAAGACC |
| | | CACTTCCCCCAGCCCTGGGAGTTCTTCGCCCAGGAGGTGATGATCCGGGTGTTCG |
| | | GCAAGCCCGACGGCAAGCCCGAGTTCGAGGAGGCCGACACCCCCGAGAAGCTGCG |
| | | GACCCTGCTGGCCGAGAAGCTGTCCTCCCGGCCCGAGGCCGTGCACGAGTACGTG |
| | | ACCCCCCTGTTCGTGTCCCGGGCCCCCAACCGGAAGATGTCCGGCGCCCACAAGG |
| | | ACACCCTGCGGTCCGCCAAGCGGTTCGTGAAGCACAACGAGAAGATCTCCGTGAA |
| | | GCGGGTGTGGCTGACCGAGATCAAGCTGGCCGACCTGGAGAACATGGTGAACTAC |
| | | AAGAACGGCCGGGAGATCGAGCTGTACGAGGCCCTGAAGGCCCGGCTGGAGGCCT |
| | | ACGGCGGCAACGCCAAGCAGGCCTTCGACCCCAAGGACAACCCCTTCTACAAGAA |
| | | GGGCGGCCAGCTGGTGAAGGCCGTGCGGGTGGAGAAGACCCAGGAGTCCGGCGTG |
| | | CTGCTGAACAAGAAGAACGCCTACACCATCGCCGACAACGGCGACATGGTGCGGG |
| | | TGGACGTGTTCTGCAAGGTGGACAAGTCCGGCGGCGGCTCCCCCAAGAAGAAGCG |
| | | GAAGGTGTCCGGCGGCTCCGGCAAGAACCAGTACTTCATCGTGCCCATCTACGCC |
| | | TGGCAGGTGGCCGAGAACATCCTGCCCGACATCGACTGCAAGGGCTACCGGATCG |
| | | ACGACTCCTACACCTTCTGCTTCTCCCTGCACAAGTACGACCTGATCGCCTTCCA |
| | | GAAGGACGAGAAGTCCAAGGTGGAGTTCGCCTACTACATCAACTGCGACTCCTCC |
| | | AACGGCCGGTTCTACCTGGCCTGGCACGACAAGGGCTCCAAGGAGCAGCAGTTCC |
| | | GGATCTCCACCCAGAACCTGGTGCTGATCCAGAAGTACCAGGTGAACGAGCTGGG |
| | | CAAGGAGATCCGGCCCTGCCGGCTGAAGAAGCGGCCCCCCGTGCGGTAG |
| 648 | Open reading | ATGGTGCCCAAGAAGAAGCGGAAGGTGGCCGCCTTCAAGCCCAACCCCATCAACT |
| | frame for | ACATCCTGGGCCTGGACATCGGCATCGCCTCCGTGGGCTGGGCCATGGTGGAGAT |
| | Nme2Cas9 | CGACGAGGAGGAGAACCCCATCCGGCTGATCGACCTGGGCGTGCGGGTGTTCGAG |
| | encoded | CGGGCCGAGGTGCCCAAGACCGGCGACTCCCTGGCCATGGCCCGGCGGCTGGCCC |
| | by mRNA I | GGTCCGTGCGGCGGCTGACCCGGCGGCGGGCCCACCGGCTGCTGCGGGCCCGGCG |
| | | GCTGCTGAAGCGGGAGGGCGTGCTGCAGGCCGCCGACTTCGACGAGAACGGCCTG |
| | | ATCAAGTCCCTGCCCAACACCCCCTGGCAGCTGCGGGCCGCCGCCCTGGACCGGA |
| | | AGCTGACCCCCCTGGAGTGGTCCGCCGTGCTGCTGCACCTGATCAAGCACCGGGG |
| | | CTACCTGTCCCAGCGGAAGAACGAGGGCGAGACCGCCGACAAGGAGCTGGGCGCC |
| | | CTGCTGAAGGGCGTGGCCAACAACGCCCACGCCCTGCAGACCGGCGACTTCCGGA |
| | | CCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTG |
| | | ATCCTGCTGTTCGAGAAGCAGAAGGAGTTCGGCAACCCCCACGTGTCCGGCGGCC |
| | | TGAAGGAGGGCATCGAGACCCTGCTGATGACCCAGCGGCCCGCCCTGTCCGGCGA |
| | | CGCCGTGCAGAAGATGCTGGGCCACTGCACCTTCGAGCCCGCCGAGCCCAAGGCC |
| | | GCCAAGAACACCTACACCGCCGAGCGGTTCATCTGGCTGACCAAGCTGAACAACC |
| | | TGCGGATCCTGGAGCAGGGCTCCGAGCGGCCCCTGACCGACACCGAGCGGGCCAC |
| | | CCTGATGGACGAGCCCTACCGGAAGTCCAAGCTGACCTACGCCCAGGCCCGGAAG |
| | | CTGCTGGGCCTGGAGGACACCGCCTTCTTCAAGGGCCTGCGGTACGGCAAGGACA |
| | | ACGCCGAGGCCTCCACCCTGATGGAGATGAAGGCCTACCACGCCATCTCCCGGGC |
| | | CCTGGAGAAGGAGGGCCTGAAGGACAAGAAGTCCCCCCTGAACCTGTCCTCCGAG |
| | | CTGCAGGACGAGATCGGCACCGCCTTCTCCCTGTTCAAGACCGACGAGGACATCA |
| | | CCGGCCGGCTGAAGGACCGGGTGCAGCCCGAGATCCTGGAGGCCCTGCTGAAGCA |
| | | CATCTCCTTCGACAAGTTCGTGCAGATCTCCCTGAAGGCCCTGCGGCGGATCGTG |
| | | CCCCTGATGGAGCAGGGCAAGCGGTACGACGAGGCCTGCGCCGAGATCTACGGCG |
| | | ACCACTACGGCAAGAAGAACACCGAGGAGAAGATCTACCTGCCCCCCATCCCCGC |
| | | CGACGAGATCCGGAACCCCGTGGTGCTGCGGGCCCTGTCCCAGGCCCGGAAGGTG |
| | | ATCAACGGCGTGGTGCGGCGGTACGGCTCCCCCGCCCGGATCCACATCGAGACCG |
| | | CCCGGGAGGTGGGCAAGTCCTTCAAGGACCGGAAGGAGATCGAGAAGCGGCAGGA |
| | | GGAGAACCGGAAGGACCGGGAGAAGGCCGCCGCCAAGTTCCGGGAGTACTTCCCC |
| | | AACTTCGTGGGCGAGCCCAAGTCCAAGGACATCCTGAAGCTGCGGCTGTACGAGC |
| | | AGCAGCACGGCAAGTGCCTGTACTCCGGCAAGGAGATCAACCTGGTGCGGCTGAA |
| | | CGAGAAGGGCTACGTGGAGATCGACCACGCCCTGCCCTTCTCCCGGACCTGGGAC |
| | | GACTCCTTCAACAACAAGGTGCTGGTGCTGGGCTCCGAGAACCAGAACAAGGGCA |
| | | ACCAGACCCCCTACGAGTACTTCAACGGCAAGGACAACTCCCGGGAGTGGCAGGA |
| | | GTTCAAGGCCCGGGTGGAGACCTCCCGGTTCCCCCGGTCCAAGAAGCAGCGGATC |
| | | CTGCTGCAGAAGTTCGACGAGGACGGCTTCAAGGAGTGCAACCTGAACGACACCC |
| | | GGTACGTGAACCGGTTCCTGTGCCAGTTCGTGGCCGACCACATCCTGCTGACCGG |
| | | CAAGGGCAAGCGGCGGGTGTTCGCCTCCAACGGCCAGATCACCAACCTGCTGCGG |
| | | GGCTTCTGGGGCCTGCGGAAGGTGCGGGCCGAGAACGACCGGCACCACGCCCTGG |
| | | ACGCCGTGGTGGTGGCCTGCTCCACCGTGGCCATGCAGCAGAAGATCACCCGGTT |
| | | CGTGCGGTACAAGGAGATGAACGCCTTCGACGGCAAGACCATCGACAAGGAGACC |
| | | GGCAAGGTGCTGCACCAGAAGACCCACTTCCCCCAGCCCTGGGAGTTCTTCGCCC |
| | | AGGAGGTGATGATCCGGGTGTTCGGCAAGCCCGACGGCAAGCCCGAGTTCGAGGA |
| | | GGCCGACACCCCCGAGAAGCTGCGGACCCTGCTGGCCGAGAAGCTGTCCTCCCGG |
| | | CCCGAGGCCGTGCACGAGTACGTGACCCCCCTGTTCGTGTCCCGGGCCCCCAACC |
| | | GGAAGATGTCCGGCGCCCACAAGGACACCCTGCGGTCCGCCAAGCGGTTCGTGAA |
| | | GCACAACGAGAAGATCTCCGTGAAGCGGGTGTGGCTGACCGAGATCAAGCTGGCC |
| | | GACCTGGAGAACATGGTGAACTACAAGAACGGCCGGGAGATCGAGCTGTACGAGG |
| | | CCCTGAAGGCCCGGCTGGAGGCCTACGGCGGCAACGCCAAGCAGGCCTTCGACCC |
| | | CAAGGACAACCCCTTCTACAAGAAGGGCGGCCAGCTGGTGAAGGCCGTGCGGGTG |
| | | GAGAAGACCCAGGAGTCCGGCGTGCTGCTGAACAAGAAGAACGCCTACACCATCG |
| | | CCGACAACGGCGACATGGTGCGGGTGGACGTGTTCTGCAAGGTGGACAAGAAGGG |
| | | CAAGAACCAGTACTTCATCGTGCCCATCTACGCCTGGCAGGTGGCCGAGAACATC |
| | | CTGCCCGACATCGACTGCAAGGGCTACCGGATCGACGACTCCTACACCTTCTGCT |
| | | TCTCCCTGCACAAGTACGACCTGATCGCCTTCCAGAAGGACGAGAAGTCCAAGGT |
| | | GGAGTTCGCCTACTACATCAACTGCGACTCCTCCAACGGCCGGTTCTACCTGGCC |
| | | TGGCACGACAAGGGCTCCAAGGAGCAGCAGTTCCGGATCTCCACCCAGAACCTGG |
| | | TGCTGATCCAGAAGTACCAGGTGAACGAGCTGGGCAAGGAGATCCGGCCCTGCCG |
| | | GCTGAAGAAGCGGCCCCCCGTGCGGTACCCCTACGACGTGCCCGACTACGCCGCC |
| | | GCCCCCGCCGCCAAGAAGAAGAAGCTGGACTAG |
| 649 | Open reading | ATGGCCGCCTTCAAGCCCAACCCCATCAACTACATCCTGGGCCTGGACATCGGCA |
| | frame for | TCGCCTCCGTGGGCTGGGCCATGGTGGAGATCGACGAGGAGGAGAACCCCATCCG |
| | Nme2Cas9 | GCTGATCGACCTGGGCGTGCGGGTGTTCGAGCGGGCCGAGGTGCCCAAGACCGGC |
| | encoded | GACTCCCTGGCCATGGCCCGGCGGCTGGCCCGGTCCGTGCGGCGGCTGACCCGGC |
| | by mRNA J | GGCGGGCCCACCGGCTGCTGCGGGCCCGGCGGCTGCTGAAGCGGGAGGGCGTGCT |
| | | GCAGGCCGCCGACTTCGACGAGAACGGCCTGATCAAGTCCCTGCCCAACACCCCC |
| | | TGGCAGCTGCGGGCCGCCGCCCTGGACCGGAAGCTGACCCCCCTGGAGTGGTCCG |
| | | CCGTGCTGCTGCACCTGATCAAGCACCGGGGCTACCTGTCCCAGCGGAAGAACGA |
| | | GGGCGAGACCGCCGACAAGGAGCTGGGCGCCCTGCTGAAGGGCGTGGCCAACAAC |
| | | GCCCACGCCCTGCAGACCGGCGACTTCCGGACCCCCGCCGAGCTGGCCCTGAACA |
| | | AGTTCGAGAAGGAGTCCGGCCACATCCGGAACCAGCGGGGCGACTACTCCCACAC |
| | | CTTCTCCCGGAAGGACCTGCAGGCCGAGCTGATCCTGCTGTTCGAGAAGCAGAAG |
| | | GAGTTCGGCAACCCCCACGTGTCCGGCGGCCTGAAGGAGGGCATCGAGACCCTGC |
| | | TGATGACCCAGCGGCCCGCCCTGTCCGGCGACGCCGTGCAGAAGATGCTGGGCCA |
| | | CTGCACCTTCGAGCCCGCCGAGCCCAAGGCCGCCAAGAACACCTACACCGCCGAG |
| | | CGGTTCATCTGGCTGACCAAGCTGAACAACCTGCGGATCCTGGAGCAGGGCTCCG |
| | | AGCGGCCCCTGACCGACACCGAGCGGGCCACCCTGATGGACGAGCCCTACCGGAA |
| | | GTCCAAGCTGACCTACGCCCAGGCCCGGAAGCTGCTGGGCCTGGAGGACACCGCC |
| | | TTCTTCAAGGGCCTGCGGTACGGCAAGGACAACGCCGAGGCCTCCACCCTGATGG |
| | | AGATGAAGGCCTACCACGCCATCTCCCGGGCCCTGGAGAAGGAGGGCCTGAAGGA |
| | | CAAGAAGTCCCCCCTGAACCTGTCCTCCGAGCTGCAGGACGAGATCGGCACCGCC |
| | | TTCTCCCTGTTCAAGACCGACGAGGACATCACCGGCCGGCTGAAGGACCGGGTGC |
| | | AGCCCGAGATCCTGGAGGCCCTGCTGAAGCACATCTCCTTCGACAAGTTCGTGCA |
| | | GATCTCCCTGAAGGCCCTGCGGCGGATCGTGCCCCTGATGGAGCAGGGCAAGCGG |
| | | TACGACGAGGCCTGCGCCGAGATCTACGGCGACCACTACGGCAAGAAGAACACCG |
| | | AGGAGAAGATCTACCTGCCCCCCATCCCCGCCGACGAGATCCGGAACCCCGTGGT |
| | | GCTGCGGGCCCTGTCCCAGGCCCGGAAGGTGATCAACGGCGTGGTGCGGCGGTAC |
| | | GGCTCCCCCGCCCGGATCCACATCGAGACCGCCCGGGAGGTGGGCAAGTCCTTCA |
| | | AGGACCGGAAGGAGATCGAGAAGCGGCAGGAGGAGAACCGGAAGGACCGGGAGAA |
| | | GGCCGCCGCCAAGTTCCGGGAGTACTTCCCCAACTTCGTGGGCGAGCCCAAGTCC |
| | | AAGGACATCCTGAAGCTGCGGCTGTACGAGCAGCAGCACGGCAAGTGCCTGTACT |
| | | CCGGCAAGGAGATCAACCTGGTGCGGCTGAACGAGAAGGGCTACGTGGAGATCGA |
| | | CCACGCCCTGCCCTTCTCCCGGACCTGGGACGACTCCTTCAACAACAAGGTGCTG |
| | | GTGCTGGGCTCCGAGAACCAGAACAAGGGCAACCAGACCCCCTACGAGTACTTCA |
| | | ACGGCAAGGACAACTCCCGGGAGTGGCAGGAGTTCAAGGCCCGGGTGGAGACCTC |
| | | CCGGTTCCCCCGGTCCAAGAAGCAGCGGATCCTGCTGCAGAAGTTCGACGAGGAC |
| | | GGCTTCAAGGAGTGCAACCTGAACGACACCCGGTACGTGAACCGGTTCCTGTGCC |
| | | AGTTCGTGGCCGACCACATCCTGCTGACCGGCAAGGGCAAGCGGCGGGTGTTCGC |
| | | CTCCAACGGCCAGATCACCAACCTGCTGCGGGGCTTCTGGGGCCTGCGGAAGGTG |
| | | CGGGCCGAGAACGACCGGCACCACGCCCTGGACGCCGTGGTGGTGGCCTGCTCCA |
| | | CCGTGGCCATGCAGCAGAAGATCACCCGGTTCGTGCGGTACAAGGAGATGAACGC |
| | | CTTCGACGGCAAGACCATCGACAAGGAGACCGGCAAGGTGCTGCACCAGAAGACC |
| | | CACTTCCCCCAGCCCTGGGAGTTCTTCGCCCAGGAGGTGATGATCCGGGTGTTCG |
| | | GCAAGCCCGACGGCAAGCCCGAGTTCGAGGAGGCCGACACCCCCGAGAAGCTGCG |
| | | GACCCTGCTGGCCGAGAAGCTGTCCTCCCGGCCCGAGGCCGTGCACGAGTACGTG |
| | | ACCCCCCTGTTCGTGTCCCGGGCCCCCAACCGGAAGATGTCCGGCGCCCACAAGG |
| | | ACACCCTGCGGTCCGCCAAGCGGTTCGTGAAGCACAACGAGAAGATCTCCGTGAA |
| | | GCGGGTGTGGCTGACCGAGATCAAGCTGGCCGACCTGGAGAACATGGTGAACTAC |
| | | AAGAACGGCCGGGAGATCGAGCTGTACGAGGCCCTGAAGGCCCGGCTGGAGGCCT |
| | | ACGGCGGCAACGCCAAGCAGGCCTTCGACCCCAAGGACAACCCCTTCTACAAGAA |
| | | GGGCGGCCAGCTGGTGAAGGCCGTGCGGGTGGAGAAGACCCAGGAGTCCGGCGTG |
| | | CTGCTGAACAAGAAGAACGCCTACACCATCGCCGACAACGGCGACATGGTGCGGG |
| | | TGGACGTGTTCTGCAAGGTGGACAAGAAGGGCAAGAACCAGTACTTCATCGTGCC |
| | | CATCTACGCCTGGCAGGTGGCCGAGAACATCCTGCCCGACATCGACTGCAAGGGC |
| | | TACCGGATCGACGACTCCTACACCTTCTGCTTCTCCCTGCACAAGTACGACCTGA |
| | | TCGCCTTCCAGAAGGACGAGAAGTCCAAGGTGGAGTTCGCCTACTACATCAACTG |
| | | CGACTCCTCCAACGGCCGGTTCTACCTGGCCTGGCACGACAAGGGCTCCAAGGAG |
| | | CAGCAGTTCCGGATCTCCACCCAGAACCTGGTGCTGATCCAGAAGTACCAGGTGA |
| | | ACGAGCTGGGCAAGGAGATCCGGCCCTGCCGGCTGAAGAAGCGGCCCCCCGTGCG |
| | | G |
| 650 | Open reading | atggccgccttcaagcccaaccccatcaactacatcctgggcctggacatcggca |
| | frame for | tcgcctccgtgggctgggccatggtggagatcgacgaggaggagaaccccatccg |
| | Nme2Cas9 | gctgatcgacctgggcgtgcgggtgttcgagcgggccgaggtgcccaagaccggc |
| | encoded | gactccctggccatggcccggcggctggcccggtccgtgcggcggctgacccggc |
| | by mRNA K | ggcgggcccaccggctgctgcgggcccggcggctgctgaagcgggagggcgtgct |
| | | gcaggccgccgacttcgacgagaacggcctgatcaagtccctgcccaacaccccc |
| | | tggcagctgcgggccgccgccctggaccggaagctgacccccctggagtggtccg |
| | | ccgtgctgctgcacctgatcaagcaccggggctacctgtcccagcggaagaacga |
| | | gggcgagaccgccgacaaggagctgggcgccctgctgaagggcgtggccaacaac |
| | | gcccacgccctgcagaccggcgacttccggacccccgccgagctggccctgaaca |
| | | agttcgagaaggagtccggccacatccggaaccagcggggcgactactcccacac |
| | | cttctcccggaaggacctgcaggccgagctgatcctgctgttcgagaagcagaag |
| | | gagttcggcaacccccacgtgtccggcggcctgaaggagggcatcgagaccctgc |
| | | tgatgacccagcggcccgccctgtccggcgacgccgtgcagaagatgctgggcca |
| | | ctgcaccttcgagcccgccgagcccaaggccgccaagaacacctacaccgccgag |
| | | cggttcatctggctgaccaagctgaacaacctgcggatcctggagcagggctccg |
| | | agcggcccctgaccgacaccgagcgggccaccctgatggacgagccctaccggaa |
| | | gtccaagctgacctacgcccaggcccggaagctgctgggcctggaggacaccgcc |
| | | ttcttcaagggcctgcggtacggcaaggacaacgccgaggcctccaccctgatgg |
| | | agatgaaggcctaccacgccatctcccgggccctggagaaggagggcctgaagga |
| | | caagaagtcccccctgaacctgtcctccgagctgcaggacgagatcggcaccgcc |
| | | ttctccctgttcaagaccgacgaggacatcaccggccggctgaaggaccgggtgc |
| | | agcccgagatcctggaggccctgctgaagcacatctccttcgacaagttcgtgca |
| | | gatctccctgaaggccctgcggcggatcgtgcccctgatggagcagggcaagcgg |
| | | tacgacgaggcctgcgccgagatctacggcgaccactacggcaagaagaacaccg |
| | | aggagaagatctacctgccccccatccccgccgacgagatccggaaccccgtggt |
| | | gctgcgggccctgtcccaggcccggaaggtgatcaacggcgtggtgcggcggtac |
| | | ggctcccccgcccggatccacatcgagaccgcccgggaggtgggcaagtccttca |
| | | aggaccggaaggagatcgagaagcggcaggaggagaaccggaaggaccgggagaa |
| | | ggccgccgccaagttccgggagtacttccccaacttcgtgggcgagcccaagtcc |
| | | aaggacatcctgaagctgcggctgtacgagcagcagcacggcaagtgcctgtact |
| | | ccggcaaggagatcaacctggtgcggctgaacgagaagggctacgtggagatcga |
| | | ccacgccctgcccttctcccggacctgggacgactccttcaacaacaaggtgctg |
| | | gtgctgggctccgagaaccagaacaagggcaaccagaccccctacgagtacttca |
| | | acggcaaggacaactcccgggagtggcaggagttcaaggcccgggggagacctcc |
| | | cggttcccccggtccaagaagcagcggatcctgctgcagaagttcgacgaggacg |
| | | gcttcaaggagtgcaacctgaacgacacccggtacgtgaaccggttcctgtgcca |
| | | gttcgtggccgaccacatcctgctgaccggcaagggcaagcggcgggtgttcgcc |
| | | tccaacggccagatcaccaacctgctgcggggcttctggggcctgcggaaggtgc |
| | | gggccgagaacgaccggcaccacgccctggacgccgtggtggtggcctgctccac |
| | | cgtggccatgcagcagaagatcacccggttcgtgcggtacaaggagatgaacgcc |
| | | ttcgacggcaagaccatcgacaaggagaccggcaaggtgctgcaccagaagaccc |
| | | acttcccccagccctgggagttcttcgcccaggaggtgatgatccgggtgttcgg |
| | | caagcccgacggcaagcccgagttcgaggaggccgacacccccgagaagctgcgg |
| | | accctgctggccgagaagctgtcctcccggcccgaggccgtgcacgagtacgtga |
| | | cccccctgttcgtgtcccgggcccccaaccggaagatgtccggcgcccacaagga |
| | | caccctgcggtccgccaagcggttcgtgaagcacaacgagaagatctccgtgaag |
| | | cgggtgtggctgaccgagatcaagctggccgacctggagaacatggtgaactaca |
| | | agaacggccgggagatcgagctgtacgaggccctgaaggcccggctggaggccta |
| | | cggcggcaacgccaagcaggccttcgaccccaaggacaaccccttctacaagaag |
| | | ggcggccagctggtgaaggccgtgcgggtggagaagacccaggagtccggcgtgc |
| | | tgctgaacaagaagaacgcctacaccatcgccgacaacggcgacatggtgcgggt |
| | | ggacgtgttctgcaaggtggacaagaagggcaagaaccagtacttcatcgtgccc |
| | | atctacgcctggcaggtggccgagaacatcctgcccgacatcgactgcaagggct |
| | | accggatcgacgactcctacaccttctgcttctccctgcacaagtacgacctgat |
| | | cgccttccagaaggacgagaagtccaaggtggagttcgcctactacatcaactgc |
| | | gactcctccaacggccggttctacctggcctggcacgacaagggctccaaggagc |
| | | agcagttccggatctccacccagaacctggtgctgatccagaagtaccaggtgaa |
| | | cgagctgggcaaggagatccggccctgccggctgaagaagcggccccccgtgcgg |
| 651 | Open reading | atgGACGGCTCCGGCGGCGGCTCCCCCAAGAAGAAGCGGAAGGTGGGCGGCTCCG |
| | frame for | GCGGCGGCgccgccttcaagcccaaccccatcaactacatcctgggcctggacat |
| | Nme2Cas9 | cggcatcgcctccgtgggctgggccatggtggagatcgacgaggaggagaacccc |
| | encoded | atccggctgatcgacctgggcgtgcgggtgttcgagcgggccgaggtgcccaaga |
| | by mRNA L | ccggcgactccctggccatggcccggcggctggcccggtccgtgcggcggctgac |
| | | ccggcggcgggcccaccggctgctgcgggcccggcggctgctgaagcgggagggc |
| | | gtgctgcaggccgccgacttcgacgagaacggcctgatcaagtccctgcccaaca |
| | | ccccctggcagctgcgggccgccgccctggaccggaagctgacccccctggagtg |
| | | gtccgccgtgctgctgcacctgatcaagcaccggggctacctgtcccagcggaag |
| | | aacgagggcgagaccgccgacaaggagctgggcgccctgctgaagggcgtggcca |
| | | acaacgcccacgccctgcagaccggcgacttccggacccccgccgagctggccct |
| | | gaacaagttcgagaaggagtccggccacatccggaaccagcggggcgactactcc |
| | | cacaccttctcccggaaggacctgcaggccgagctgatcctgctgttcgagaagc |
| | | agaaggagttcggcaacccccacgtgtccggcggcctgaaggagggcatcgagac |
| | | cctgctgatgacccagcggcccgccctgtccggcgacgccgtgcagaagatgctg |
| | | ggccactgcaccttcgagcccgccgagcccaaggccgccaagaacacctacaccg |
| | | ccgagcggttcatctggctgaccaagctgaacaacctgcggatcctggagcaggg |
| | | ctccgagcggcccctgaccgacaccgagcgggccaccctgatggacgagccctac |
| | | cggaagtccaagctgacctacgcccaggcccggaagctgctgggcctggaggaca |
| | | ccgccttcttcaagggcctgcggtacggcaaggacaacgccgaggcctccaccct |
| | | gatggagatgaaggcctaccacgccatctcccgggccctggagaaggagggcctg |
| | | aaggacaagaagtcccccctgaacctgtcctccgagctgcaggacgagatcggca |
| | | ccgccttctccctgttcaagaccgacgaggacatcaccggccggctgaaggaccg |
| | | ggtgcagcccgagatcctggaggccctgctgaagcacatctccttcgacaagttc |
| | | gtgcagatctccctgaaggccctgcggcggatcgtgcccctgatggagcagggca |
| | | agcggtacgacgaggcctgcgccgagatctacggcgaccactacggcaagaagaa |
| | | caccgaggagaagatctacctgccccccatccccgccgacgagatccggaacccc |
| | | gtggtgctgcgggccctgtcccaggcccggaaggtgatcaacggcgtggtgcggc |
| | | ggtacggctcccccgcccggatccacatcgagaccgcccgggaggtgggcaagtc |
| | | cttcaaggaccggaaggagatcgagaagcggcaggaggagaaccggaaggaccgg |
| | | gagaaggccgccgccaagttccgggagtacttccccaacttcgtgggcgagccca |
| | | agtccaaggacatcctgaagctgcggctgtacgagcagcagcacggcaagtgcct |
| | | gtactccggcaaggagatcaacctggtgcggctgaacgagaagggctacgtggag |
| | | atcgaccacgccctgcccttctcccggacctgggacgactccttcaacaacaagg |
| | | tgctggtgctgggctccgagaaccagaacaagggcaaccagaccccctacgagta |
| | | cttcaacggcaaggacaactcccgggagtggcaggagttcaaggcccgggtggag |
| | | acctcccggttcccccggtccaagaagcagcggatcctgctgcagaagttcgacg |
| | | aggacggcttcaaggagtgcaacctgaacgacacccggtacgtgaaccggttcct |
| | | gtgccagttcgtggccgaccacatcctgctgaccggcaagggcaagcggcgggtg |
| | | ttcgcctccaacggccagatcaccaacctgctgcggggcttctggggcctgcgga |
| | | aggtgcgggccgagaacgaccggcaccacgccctggacgccgtggtggtggcctg |
| | | ctccaccgtggccatgcagcagaagatcacccggttcgtgcggtacaaggagatg |
| | | aacgccttcgacggcaagaccatcgacaaggagaccggcaaggtgctgcaccaga |
| | | agacccacttcccccagccctgggagttcttcgcccaggaggtgatgatccgggt |
| | | gttcggcaagcccgacggcaagcccgagttcgaggaggccgacacccccgagaag |
| | | ctgcggaccctgctggccgagaagctgtcctcccggcccgaggccgtgcacgagt |
| | | acgtgacccccctgttcgtgtcccgggcccccaaccggaagatgtccggcgccca |
| | | caaggacaccctgcggtccgccaagcggttcgtgaagcacaacgagaagatctcc |
| | | gtgaagcgggtgtggctgaccgagatcaagctggccgacctggagaacatggtga |
| | | actacaagaacggccgggagatcgagctgtacgaggccctgaaggcccggctgga |
| | | ggcctacggcggcaacgccaagcaggccttcgaccccaaggacaaccccttctac |
| | | aagaagggcggccagctggtgaaggccgtgcgggtggagaagacccaggagtccg |
| | | gcgtgctgctgaacaagaagaacgcctacaccatcgccgacaacggcgacatggt |
| | | gcgggtggacgtgttctgcaaggtggacaagaagggcaagaaccagtacttcatc |
| | | gtgcccatctacgcctggcaggtggccgagaacatcctgcccgacatcgactgca |
| | | agggctaccggatcgacgactcctacaccttctgcttctccctgcacaagtacga |
| | | cctgatcgccttccagaaggacgagaagtccaaggtggagttcgcctactacatc |
| | | aactgcgactcctccaacggccggttctacctggcctggcacgacaagggctcca |
| | | aggagcagcagttccggatctccacccagaacctggtgctgatccagaagtacca |
| | | ggtgaacgagctgggcaaggagatccggccctgccggctgaagaagcggcccccc |
| | | gtgcggtag |
| 652 | Open reading | ATGGACGGCTCCGGCGGCGGCTCCCCCAAGAAGAAGCGGAAGGTGGGCGGCTCCG |
| | frame for | GCGGCGGCGCCGCCTTCAAGCCCAACCCCATCAACTACATCCTGGGCCTGGACAT |
| | Nme2Cas9 with | CGGCATCGCCTCCGTGGGCTGGGCCATGGTGGAGATCGACGAGGAGGAGAACCCC |
| | HiBiT tag | ATCCGGCTGATCGACCTGGGCGTGCGGGTGTTCGAGCGGGCCGAGGTGCCCAAGA |
| | encoded by mRNA | CCGGCGACTCCCTGGCCATGGCCCGGCGGCTGGCCCGGTCCGTGCGGCGGCTGAC |
| | M | CCGGCGGCGGGCCCACCGGCTGCTGCGGGCCCGGCGGCTGCTGAAGCGGGAGGGC |
| | | GTGCTGCAGGCCGCCGACTTCGACGAGAACGGCCTGATCAAGTCCCTGCCCAACA |
| | | CCCCCTGGCAGCTGCGGGCCGCCGCCCTGGACCGGAAGCTGACCCCCCTGGAGTG |
| | | GTCCGCCGTGCTGCTGCACCTGATCAAGCACCGGGGCTACCTGTCCCAGCGGAAG |
| | | AACGAGGGCGAGACCGCCGACAAGGAGCTGGGCGCCCTGCTGAAGGGCGTGGCCA |
| | | ACAACGCCCACGCCCTGCAGACCGGCGACTTCCGGACCCCCGCCGAGCTGGCCCT |
| | | GAACAAGTTCGAGAAGGAGTCCGGCCACATCCGGAACCAGCGGGGCGACTACTCC |
| | | CACACCTTCTCCCGGAAGGACCTGCAGGCCGAGCTGATCCTGCTGTTCGAGAAGC |
| | | AGAAGGAGTTCGGCAACCCCCACGTGTCCGGCGGCCTGAAGGAGGGCATCGAGAC |
| | | CCTGCTGATGACCCAGCGGCCCGCCCTGTCCGGCGACGCCGTGCAGAAGATGCTG |
| | | GGCCACTGCACCTTCGAGCCCGCCGAGCCCAAGGCCGCCAAGAACACCTACACCG |
| | | CCGAGCGGTTCATCTGGCTGACCAAGCTGAACAACCTGCGGATCCTGGAGCAGGG |
| | | CTCCGAGCGGCCCCTGACCGACACCGAGCGGGCCACCCTGATGGACGAGCCCTAC |
| | | CGGAAGTCCAAGCTGACCTACGCCCAGGCCCGGAAGCTGCTGGGCCTGGAGGACA |
| | | CCGCCTTCTTCAAGGGCCTGCGGTACGGCAAGGACAACGCCGAGGCCTCCACCCT |
| | | GATGGAGATGAAGGCCTACCACGCCATCTCCCGGGCCCTGGAGAAGGAGGGCCTG |
| | | AAGGACAAGAAGTCCCCCCTGAACCTGTCCTCCGAGCTGCAGGACGAGATCGGCA |
| | | CCGCCTTCTCCCTGTTCAAGACCGACGAGGACATCACCGGCCGGCTGAAGGACCG |
| | | GGTGCAGCCCGAGATCCTGGAGGCCCTGCTGAAGCACATCTCCTTCGACAAGTTC |
| | | GTGCAGATCTCCCTGAAGGCCCTGCGGCGGATCGTGCCCCTGATGGAGCAGGGCA |
| | | AGCGGTACGACGAGGCCTGCGCCGAGATCTACGGCGACCACTACGGCAAGAAGAA |
| | | CACCGAGGAGAAGATCTACCTGCCCCCCATCCCCGCCGACGAGATCCGGAACCCC |
| | | GTGGTGCTGCGGGCCCTGTCCCAGGCCCGGAAGGTGATCAACGGCGTGGTGCGGC |
| | | GGTACGGCTCCCCCGCCCGGATCCACATCGAGACCGCCCGGGAGGTGGGCAAGTC |
| | | CTTCAAGGACCGGAAGGAGATCGAGAAGCGGCAGGAGGAGAACCGGAAGGACCGG |
| | | GAGAAGGCCGCCGCCAAGTTCCGGGAGTACTTCCCCAACTTCGTGGGCGAGCCCA |
| | | AGTCCAAGGACATCCTGAAGCTGCGGCTGTACGAGCAGCAGCACGGCAAGTGCCT |
| | | GTACTCCGGCAAGGAGATCAACCTGGTGCGGCTGAACGAGAAGGGCTACGTGGAG |
| | | ATCGACCACGCCCTGCCCTTCTCCCGGACCTGGGACGACTCCTTCAACAACAAGG |
| | | TGCTGGTGCTGGGCTCCGAGAACCAGAACAAGGGCAACCAGACCCCCTACGAGTA |
| | | CTTCAACGGCAAGGACAACTCCCGGGAGTGGCAGGAGTTCAAGGCCCGGGTGGAG |
| | | ACCTCCCGGTTCCCCCGGTCCAAGAAGCAGCGGATCCTGCTGCAGAAGTTCGACG |
| | | AGGACGGCTTCAAGGAGTGCAACCTGAACGACACCCGGTACGTGAACCGGTTCCT |
| | | GTGCCAGTTCGTGGCCGACCACATCCTGCTGACCGGCAAGGGCAAGCGGCGGGTG |
| | | TTCGCCTCCAACGGCCAGATCACCAACCTGCTGCGGGGCTTCTGGGGCCTGCGGA |
| | | AGGTGCGGGCCGAGAACGACCGGCACCACGCCCTGGACGCCGTGGTGGTGGCCTG |
| | | CTCCACCGTGGCCATGCAGCAGAAGATCACCCGGTTCGTGCGGTACAAGGAGATG |
| | | AACGCCTTCGACGGCAAGACCATCGACAAGGAGACCGGCAAGGTGCTGCACCAGA |
| | | AGACCCACTTCCCCCAGCCCTGGGAGTTCTTCGCCCAGGAGGTGATGATCCGGGT |
| | | GTTCGGCAAGCCCGACGGCAAGCCCGAGTTCGAGGAGGCCGACACCCCCGAGAAG |
| | | CTGCGGACCCTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTCCGGCGCCCA |
| | | CAAGGACACCCTGCGGTCCGCCAAGCGGTTCGTGAAGCACAACGAGAAGATCTCC |
| | | GTGAAGCGGGTGTGGCTGACCGAGATCAAGCTGGCCGACCTGGAGAACATGGTGA |
| | | ACTACAAGAACGGCCGGGAGATCGAGCTGTACGAGGCCCTGAAGGCCCGGCTGGA |
| | | GGCCTACGGCGGCAACGCCAAGCAGGCCTTCGACCCCAAGGACAACCCCTTCTAC |
| | | AAGAAGGGCGGCCAGCTGGTGAAGGCCGTGCGGGTGGAGAAGACCCAGGAGTCCG |
| | | GCGTGCTGCTGAACAAGAAGAACGCCTACACCATCGCCGACAACGGCGACATGGT |
| | | GCGGGTGGACGTGTTCTGCAAGGTGGACAAGAAGGGCAAGAACCAGTACTTCATC |
| | | GTGCCCATCTACGCCTGGCAGGTGGCCGAGAACATCCTGCCCGACATCGACTGCA |
| | | AGGGCTACCGGATCGACGACTCCTACACCTTCTGCTTCTCCCTGCACAAGTACGA |
| | | CCTGATCGCCTTCCAGAAGGACGAGAAGTCCAAGGTGGAGTTCGCCTACTACATC |
| | | AACTGCGACTCCTCCAACGGCCGGTTCTACCTGGCCTGGCACGACAAGGGCTCCA |
| | | AGGAGCAGCAGTTCCGGATCTCCACCCAGAACCTGGTGCTGATCCAGAAGTACCA |
| | | GGTGAACGAGCTGGGCAAGGAGATCCGGCCCTGCCGGCTGAAGAAGCGGCCCCCC |
| | | GTGCGGTCCGAGTCCGCCACCCCCGAGTCCGTGTCCGGCTGGCGGCTGTTCAAGA |
| | | AGATCTCCTAG |
| 653 | Open reading | atgGACGGCTCCGGCGGCGGCTCCCCCAAGAAGAAGCGGAAGGTGGGCGGCTCCG |
| | frame for | GCGGCGGCgccgccttcaagcccaaccccatcaactacatcctgggcctggacat |
| | Nme2Cas9 | cggcatcgcctccgtgggctgggccatggtggagatcgacgaggaggagaacccc |
| | encoded | atccggctgatcgacctgggcgtgcgggtgttcgagcgggccgaggtgcccaaga |
| | by mRNA N | ccggcgactccctggccatggcccggcggctggcccggtccgtgcggcggctgac |
| | | ccggcggcgggcccaccggctgctgcgggcccggcggctgctgaagcgggagggc |
| | | gtgctgcaggccgccgacttcgacgagaacggcctgatcaagtccctgcccaaca |
| | | ccccctggcagctgcgggccgccgccctggaccggaagctgacccccctggagtg |
| | | gtccgccgtgctgctgcacctgatcaagcaccggggctacctgtcccagcggaag |
| | | aacgagggcgagaccgccgacaaggagctgggcgccctgctgaagggcgtggcca |
| | | acaacgcccacgccctgcagaccggcgacttccggacccccgccgagctggccct |
| | | gaacaagttcgagaaggagtccggccacatccggaaccagcggggcgactactcc |
| | | cacaccttctcccggaaggacctgcaggccgagctgatcctgctgttcgagaagc |
| | | agaaggagttcggcaacccccacgtgtccggcggcctgaaggagggcatcgagac |
| | | cctgctgatgacccagcggcccgccctgtccggcgacgccgtgcagaagatgctg |
| | | ggccactgcaccttcgagcccgccgagcccaaggccgccaagaacacctacaccg |
| | | ccgagcggttcatctggctgaccaagctgaacaacctgcggatcctggagcaggg |
| | | ctccgagcggcccctgaccgacaccgagcgggccaccctgatggacgagccctac |
| | | cggaagtccaagctgacctacgcccaggcccggaagctgctgggcctggaggaca |
| | | ccgccttcttcaagggcctgcggtacggcaaggacaacgccgaggcctccaccct |
| | | gatggagatgaaggcctaccacgccatctcccgggccctggagaaggagggcctg |
| | | aaggacaagaagtcccccctgaacctgtcctccgagctgcaggacgagatcggca |
| | | ccgccttctccctgttcaagaccgacgaggacatcaccggccggctgaaggaccg |
| | | ggtgcagcccgagatcctggaggccctgctgaagcacatctccttcgacaagttc |
| | | gtgcagatctccctgaaggccctgcggcggatcgtgcccctgatggagcagggca |
| | | agcggtacgacgaggcctgcgccgagatctacggcgaccactacggcaagaagaa |
| | | caccgaggagaagatctacctgccccccatccccgccgacgagatccggaacccc |
| | | gtggtgctgcgggccctgtcccaggcccggaaggtgatcaacggcgtggtgcggc |
| | | ggtacggctcccccgcccggatccacatcgagaccgcccgggaggtgggcaagtc |
| | | cttcaaggaccggaaggagatcgagaagcggcaggaggagaaccggaaggaccgg |
| | | gagaaggccgccgccaagttccgggagtacttccccaacttcgtgggcgagccca |
| | | agtccaaggacatcctgaagctgcggctgtacgagcagcagcacggcaagtgcct |
| | | gtactccggcaaggagatcaacctggtgcggctgaacgagaagggctacgtggag |
| | | atcgaccacgccctgcccttctcccggacctgggacgactccttcaacaacaagg |
| | | tgctggtgctgggctccgagaaccagaacaagggcaaccagaccccctacgagta |
| | | cttcaacggcaaggacaactcccgggagtggcaggagttcaaggcccgggtggag |
| | | acctcccggttcccccggtccaagaagcagcggatcctgctgcagaagttcgacg |
| | | aggacggcttcaaggagtgcaacctgaacgacacccggtacgtgaaccggttcct |
| | | gtgccagttcgtggccgaccacatcctgctgaccggcaagggcaagcggcgggtg |
| | | ttcgcctccaacggccagatcaccaacctgctgcggggcttctggggcctgcgga |
| | | aggtgcgggccgagaacgaccggcaccacgccctggacgccgtggtggtggcctg |
| | | ctccaccgtggccatgcagcagaagatcacccggttcgtgcggtacaaggagatg |
| | | aacgccttcgacggcaagaccatcgacaaggagaccggcaaggtgctgcaccaga |
| | | agacccacttcccccagccctgggagttcttcgcccaggaggtgatgatccgggt |
| | | gttcggcaagcccgacggcaagcccgagttcgaggaggccgacacccccgagaag |
| | | ctgcggaccctgctggccgagaagctgtcctcccggcccgaggccgtgcacgagt |
| | | acgtgacccccctgttcgtgtcccgggcccccaaccggaagatgtccggcgccca |
| | | caaggacaccctgcggtccgccaagcggttcgtgaagcacaacgagaagatctcc |
| | | gtgaagcgggtgtggctgaccgagatcaagctggccgacctggagaacatggtga |
| | | actacaagaacggccgggagatcgagctgtacgaggccctgaaggcccggctgga |
| | | ggcctacggcggcaacgccaagcaggccttcgaccccaaggacaaccccttctac |
| | | aagaagggcggccagctggtgaaggccgtgcgggtggagaagacccaggagtccg |
| | | gcgtgctgctgaacaagaagaacgcctacaccatcgccgacaacggcgacatggt |
| | | gcgggtggacgtgttctgcaaggtggacaagaagggcaagaaccagtacttcatc |
| | | gtgcccatctacgcctggcaggtggccgagaacatcctgcccgacatcgactgca |
| | | agggctaccggatcgacgactcctacaccttctgcttctccctgcacaagtacga |
| | | cctgatcgccttccagaaggacgagaagtccaaggtggagttcgcctactacatc |
| | | aactgcgactcctccaacggccggttctacctggcctggcacgacaagggctcca |
| | | aggagcagcagttccggatctccacccagaacctggtgctgatccagaagtacca |
| | | ggtgaacgagctgggcaaggagatccggccctgccggctgaagaagcggcccccc |
| | | gtgcggTCCGGAAAGCGGACCGCCGACGGCTCCGGAGGAGGAAGCCCCGCCGCCA |
| | | AGAAGAAGAAGCTGGACtag |
| 654 | Open reading | atgGACGGCTCCGGCGGCGGCTCCCCCAAGAAGAAGCGGAAGGTGGAGGACAAGC |
| | frame for | GGCCCGCCGCCACCAAGAAGGCCGGCCAGGCCAAGAAGAAGAAGGGCGGCTCCGG |
| | Nme2Cas9 | CGGCGGCgccgccttcaagcccaaccccatcaactacatcctgggcctggacatc |
| | encoded | ggcatcgcctccgtgggctgggccatggtggagatcgacgaggaggagaacccca |
| | by mRNA O | tccggctgatcgacctgggcgtgcgggtgttcgagcgggccgaggtgcccaagac |
| | | cggcgactccctggccatggcccggcggctggcccggtccgtgcggcggctgacc |
| | | cggcggcgggcccaccggctgctgcgggcccggcggctgctgaagcgggagggcg |
| | | tgctgcaggccgccgacttcgacgagaacggcctgatcaagtccctgcccaacac |
| | | cccctggcagctgcgggccgccgccctggaccggaagctgacccccctggagtgg |
| | | tccgccgtgctgctgcacctgatcaagcaccggggctacctgtcccagcggaaga |
| | | acgagggcgagaccgccgacaaggagctgggcgccctgctgaagggcgtggccaa |
| | | caacgcccacgccctgcagaccggcgacttccggacccccgccgagctggccctg |
| | | aacaagttcgagaaggagtccggccacatccggaaccagcggggcgactactccc |
| | | acaccttctcccggaaggacctgcaggccgagctgatcctgctgttcgagaagca |
| | | gaaggagttcggcaacccccacgtgtccggcggcctgaaggagggcatcgagacc |
| | | ctgctgatgacccagcggcccgccctgtccggcgacgccgtgcagaagatgctgg |
| | | gccactgcaccttcgagcccgccgagcccaaggccgccaagaacacctacaccgc |
| | | cgagcggttcatctggctgaccaagctgaacaacctgcggatcctggagcagggc |
| | | tccgagcggcccctgaccgacaccgagcgggccaccctgatggacgagccctacc |
| | | ggaagtccaagctgacctacgcccaggcccggaagctgctgggcctggaggacac |
| | | cgccttcttcaagggcctgcggtacggcaaggacaacgccgaggcctccaccctg |
| | | atggagatgaaggcctaccacgccatctcccgggccctggagaaggagggcctga |
| | | aggacaagaagtcccccctgaacctgtcctccgagctgcaggacgagatcggcac |
| | | cgccttctccctgttcaagaccgacgaggacatcaccggccggctgaaggaccgg |
| | | gtgcagcccgagatcctggaggccctgctgaagcacatctccttcgacaagttcg |
| | | tgcagatctccctgaaggccctgcggcggatcgtgcccctgatggagcagggcaa |
| | | gcggtacgacgaggcctgcgccgagatctacggcgaccactacggcaagaagaac |
| | | accgaggagaagatctacctgccccccatccccgccgacgagatccggaaccccg |
| | | tggtgctgcgggccctgtcccaggcccggaaggtgatcaacggcgtggtgcggcg |
| | | gtacggctcccccgcccggatccacatcgagaccgcccgggaggtgggcaagtcc |
| | | ttcaaggaccggaaggagatcgagaagcggcaggaggagaaccggaaggaccggg |
| | | agaaggccgccgccaagttccgggagtacttccccaacttcgtgggcgagcccaa |
| | | gtccaaggacatcctgaagctgcggctgtacgagcagcagcacggcaagtgcctg |
| | | tactccggcaaggagatcaacctggtgcggctgaacgagaagggctacgtggaga |
| | | tcgaccacgccctgcccttctcccggacctgggacgactccttcaacaacaaggt |
| | | gctggtgctgggctccgagaaccagaacaagggcaaccagaccccctacgagtac |
| | | ttcaacggcaaggacaactcccgggagtggcaggagttcaaggcccgggtggaga |
| | | cctcccggttcccccggtccaagaagcagcggatcctgctgcagaagttcgacga |
| | | ggacggcttcaaggagtgcaacctgaacgacacccggtacgtgaaccggttcctg |
| | | tgccagttcgtggccgaccacatcctgctgaccggcaagggcaagcggcgggtgt |
| | | tcgcctccaacggccagatcaccaacctgctgcggggcttctggggcctgcggaa |
| | | ggtgcgggccgagaacgaccggcaccacgccctggacgccgtggtggtggcctgc |
| | | tccaccgtggccatgcagcagaagatcacccggttcgtgcggtacaaggagatga |
| | | acgccttcgacggcaagaccatcgacaaggagaccggcaaggtgctgcaccagaa |
| | | gacccacttcccccagccctgggagttcttcgcccaggaggtgatgatccgggtg |
| | | ttcggcaagcccgacggcaagcccgagttcgaggaggccgacacccccgagaagc |
| | | tgcggaccctgctggccgagaagctgtcctcccggcccgaggccgtgcacgagta |
| | | cgtgacccccctgttcgtgtcccgggcccccaaccggaagatgtccggcgcccac |
| | | aaggacaccctgcggtccgccaagcggttcgtgaagcacaacgagaagatctccg |
| | | tgaagcgggtgtggctgaccgagatcaagctggccgacctggagaacatggtgaa |
| | | ctacaagaacggccgggagatcgagctgtacgaggccctgaaggcccggctggag |
| | | gcctacggcggcaacgccaagcaggccttcgaccccaaggacaaccccttctaca |
| | | agaagggcggccagctggtgaaggccgtgcgggtggagaagacccaggagtccgg |
| | | cgtgctgctgaacaagaagaacgcctacaccatcgccgacaacggcgacatggtg |
| | | cgggtggacgtgttctgcaaggtggacaagaagggcaagaaccagtacttcatcg |
| | | tgcccatctacgcctggcaggtggccgagaacatcctgcccgacatcgactgcaa |
| | | gggctaccggatcgacgactcctacaccttctgcttctccctgcacaagtacgac |
| | | ctgatcgccttccagaaggacgagaagtccaaggtggagttcgcctactacatca |
| | | actgcgactcctccaacggccggttctacctggcctggcacgacaagggctccaa |
| | | ggagcagcagttccggatctccacccagaacctggtgctgatccagaagtaccag |
| | | gtgaacgagctgggcaaggagatccggccctgccggctgaagaagcggccccccg |
| | | tgcggtag |
| 655 | Open reading | atgGACGGCTCCGGCGGCGGCTCCCCCAAGAAGAAGCGGAAGGTGGAGGACAAGC |
| | frame for | GGCCCGCCGCCACCAAGAAGGCCGGCCAGGCCAAGAAGAAGAAGGGCGGCTCCGG |
| | Nme2Cas9 with | CGGCGGCGCCGCCTTCAAGCCCAACCCCATCAACTACATCCTGGGCCTGGACATC |
| | HiBiT tag | GGCATCGCCTCCGTGGGCTGGGCCATGGTGGAGATCGACGAGGAGGAGAACCCCA |
| | encoded by | TCCGGCTGATCGACCTGGGCGTGCGGGTGTTCGAGCGGGCCGAGGTGCCCAAGAC |
| | mRNA P | CGGCGACTCCCTGGCCATGGCCCGGCGGCTGGCCCGGTCCGTGCGGCGGCTGACC |
| | | CGGCGGCGGGCCCACCGGCTGCTGCGGGCCCGGCGGCTGCTGAAGCGGGAGGGCG |
| | | TGCTGCAGGCCGCCGACTTCGACGAGAACGGCCTGATCAAGTCCCTGCCCAACAC |
| | | CCCCTGGCAGCTGCGGGCCGCCGCCCTGGACCGGAAGCTGACCCCCCTGGAGTGG |
| | | TCCGCCGTGCTGCTGCACCTGATCAAGCACCGGGGCTACCTGTCCCAGCGGAAGA |
| | | ACGAGGGCGAGACCGCCGACAAGGAGCTGGGCGCCCTGCTGAAGGGCGTGGCCAA |
| | | CAACGCCCACGCCCTGCAGACCGGCGACTTCCGGACCCCCGCCGAGCTGGCCCTG |
| | | AACAAGTTCGAGAAGGAGTCCGGCCACATCCGGAACCAGCGGGGCGACTACTCCC |
| | | ACACCTTCTCCCGGAAGGACCTGCAGGCCGAGCTGATCCTGCTGTTCGAGAAGCA |
| | | GAAGGAGTTCGGCAACCCCCACGTGTCCGGCGGCCTGAAGGAGGGCATCGAGACC |
| | | CTGCTGATGACCCAGCGGCCCGCCCTGTCCGGCGACGCCGTGCAGAAGATGCTGG |
| | | GCCACTGCACCTTCGAGCCCGCCGAGCCCAAGGCCGCCAAGAACACCTACACCGC |
| | | CGAGCGGTTCATCTGGCTGACCAAGCTGAACAACCTGCGGATCCTGGAGCAGGGC |
| | | TCCGAGCGGCCCCTGACCGACACCGAGCGGGCCACCCTGATGGACGAGCCCTACC |
| | | GGAAGTCCAAGCTGACCTACGCCCAGGCCCGGAAGCTGCTGGGCCTGGAGGACAC |
| | | CGCCTTCTTCAAGGGCCTGCGGTACGGCAAGGACAACGCCGAGGCCTCCACCCTG |
| | | ATGGAGATGAAGGCCTACCACGCCATCTCCCGGGCCCTGGAGAAGGAGGGCCTGA |
| | | AGGACAAGAAGTCCCCCCTGAACCTGTCCTCCGAGCTGCAGGACGAGATCGGCAC |
| | | CGCCTTCTCCCTGTTCAAGACCGACGAGGACATCACCGGCCGGCTGAAGGACCGG |
| | | GTGCAGCCCGAGATCCTGGAGGCCCTGCTGAAGCACATCTCCTTCGACAAGTTCG |
| | | TGCAGATCTCCCTGAAGGCCCTGCGGCGGATCGTGCCCCTGATGGAGCAGGGCAA |
| | | GCGGTACGACGAGGCCTGCGCCGAGATCTACGGCGACCACTACGGCAAGAAGAAC |
| | | ACCGAGGAGAAGATCTACCTGCCCCCCATCCCCGCCGACGAGATCCGGAACCCCG |
| | | TGGTGCTGCGGGCCCTGTCCCAGGCCCGGAAGGTGATCAACGGCGTGGTGCGGCG |
| | | GTACGGCTCCCCCGCCCGGATCCACATCGAGACCGCCCGGGAGGTGGGCAAGTCC |
| | | TTCAAGGACCGGAAGGAGATCGAGAAGCGGCAGGAGGAGAACCGGAAGGACCGGG |
| | | AGAAGGCCGCCGCCAAGTTCCGGGAGTACTTCCCCAACTTCGTGGGCGAGCCCAA |
| | | GTCCAAGGACATCCTGAAGCTGCGGCTGTACGAGCAGCAGCACGGCAAGTGCCTG |
| | | TACTCCGGCAAGGAGATCAACCTGGTGCGGCTGAACGAGAAGGGCTACGTGGAGA |
| | | TCGACCACGCCCTGCCCTTCTCCCGGACCTGGGACGACTCCTTCAACAACAAGGT |
| | | GCTGGTGCTGGGCTCCGAGAACCAGAACAAGGGCAACCAGACCCCCTACGAGTAC |
| | | TTCAACGGCAAGGACAACTCCCGGGAGTGGCAGGAGTTCAAGGCCCGGGTGGAGA |
| | | CCTCCCGGTTCCCCCGGTCCAAGAAGCAGCGGATCCTGCTGCAGAAGTTCGACGA |
| | | GGACGGCTTCAAGGAGTGCAACCTGAACGACACCCGGTACGTGAACCGGTTCCTG |
| | | TGCCAGTTCGTGGCCGACCACATCCTGCTGACCGGCAAGGGCAAGCGGCGGGTGT |
| | | TCGCCTCCAACGGCCAGATCACCAACCTGCTGCGGGGCTTCTGGGGCCTGCGGAA |
| | | GGTGCGGGCCGAGAACGACCGGCACCACGCCCTGGACGCCGTGGTGGTGGCCTGC |
| | | TCCACCGTGGCCATGCAGCAGAAGATCACCCGGTTCGTGCGGTACAAGGAGATGA |
| | | ACGCCTTCGACGGCAAGACCATCGACAAGGAGACCGGCAAGGTGCTGCACCAGAA |
| | | GACCCACTTCCCCCAGCCCTGGGAGTTCTTCGCCCAGGAGGTGATGATCCGGGTG |
| | | TTCGGCAAGCCCGACGGCAAGCCCGAGTTCGAGGAGGCCGACACCCCCGAGAAGC |
| | | TGCGGACCCTGCTGGCCGAGAAGCTGTCCTCCCGGCCCGAGGCCGTGCACGAGTA |
| | | CGTGACCCCCCTGTTCGTGTCCCGGGCCCCCAACCGGAAGATGTCCGGCGCCCAC |
| | | AAGGACACCCTGCGGTCCGCCAAGCGGTTCGTGAAGCACAACGAGAAGATCTCCG |
| | | TGAAGCGGGTGTGGCTGACCGAGATCAAGCTGGCCGACCTGGAGAACATGGTGAA |
| | | CTACAAGAACGGCCGGGAGATCGAGCTGTACGAGGCCCTGAAGGCCCGGCTGGAG |
| | | GCCTACGGCGGCAACGCCAAGCAGGCCTTCGACCCCAAGGACAACCCCTTCTACA |
| | | AGAAGGGCGGCCAGCTGGTGAAGGCCGTGCGGGTGGAGAAGACCCAGGAGTCCGG |
| | | CGTGCTGCTGAACAAGAAGAACGCCTACACCATCGCCGACAACGGCGACATGGTG |
| | | CGGGTGGACGTGTTCTGCAAGGTGGACAAGAAGGGCAAGAACCAGTACTTCATCG |
| | | TGCCCATCTACGCCTGGCAGGTGGCCGAGAACATCCTGCCCGACATCGACTGCAA |
| | | GGGCTACCGGATCGACGACTCCTACACCTTCTGCTTCTCCCTGCACAAGTACGAC |
| | | CTGATCGCCTTCCAGAAGGACGAGAAGTCCAAGGTGGAGTTCGCCTACTACATCA |
| | | ACTGCGACTCCTCCAACGGCCGGTTCTACCTGGCCTGGCACGACAAGGGCTCCAA |
| | | GGAGCAGCAGTTCCGGATCTCCACCCAGAACCTGGTGCTGATCCAGAAGTACCAG |
| | | GTGAACGAGCTGGGCAAGGAGATCCGGCCCTGCCGGCTGAAGAAGCGGCCCCCCG |
| | | TGCGGTCCGAGTCCGCCACCCCCGAGTCCGTGTCCGGCTGGCGGCTGTTCAAGAA |
| | | GATCTCCTAG |
| 656 | Open reading | atgGACGGCTCCGGCGGCGGCTCCGAGGACAAGCGGCCCGCCGCCACCAAGAAGG |
| | frame for | CCGGCCAGGCCAAGAAGAAGAAGGGCGGCTCCGGCGGCGGCgccgccttcaagcc |
| | Nme2Cas9 | caaccccatcaactacatcctgggcctggacatcggcatcgcctccgtgggctgg |
| | encoded | gccatggtggagatcgacgaggaggagaaccccatccggctgatcgacctgggcg |
| | by mRNA Q | tgcgggtgttcgagcgggccgaggtgcccaagaccggcgactccctggccatggc |
| | | ccggcggctggcccggtccgtgcggcggctgacccggcggcgggcccaccggctg |
| | | ctgcgggcccggcggctgctgaagcgggagggcgtgctgcaggccgccgacttcg |
| | | acgagaacggcctgatcaagtccctgcccaacaccccctggcagctgcgggccgc |
| | | cgccctggaccggaagctgacccccctggagtggtccgccgtgctgctgcacctg |
| | | atcaagcaccggggctacctgtcccagcggaagaacgagggcgagaccgccgaca |
| | | aggagctgggcgccctgctgaagggcgtggccaacaacgcccacgccctgcagac |
| | | cggcgacttccggacccccgccgagctggccctgaacaagttcgagaaggagtcc |
| | | ggccacatccggaaccagcggggcgactactcccacaccttctcccggaaggacc |
| | | tgcaggccgagctgatcctgctgttcgagaagcagaaggagttcggcaaccccca |
| | | cgtgtccggcggcctgaaggagggcatcgagaccctgctgatgacccagcggccc |
| | | gccctgtccggcgacgccgtgcagaagatgctgggccactgcaccttcgagcccg |
| | | ccgagcccaaggccgccaagaacacctacaccgccgagcggttcatctggctgac |
| | | caagctgaacaacctgcggatcctggagcagggctccgagcggcccctgaccgac |
| | | accgagcgggccaccctgatggacgagccctaccggaagtccaagctgacctacg |
| | | cccaggcccggaagctgctgggcctggaggacaccgccttcttcaagggcctgcg |
| | | gtacggcaaggacaacgccgaggcctccaccctgatggagatgaaggcctaccac |
| | | gccatctcccgggccctggagaaggagggcctgaaggacaagaagtcccccctga |
| | | acctgtcctccgagctgcaggacgagatcggcaccgccttctccctgttcaagac |
| | | cgacgaggacatcaccggccggctgaaggaccgggtgcagcccgagatcctggag |
| | | gccctgctgaagcacatctccttcgacaagttcgtgcagatctccctgaaggccc |
| | | tgcggcggatcgtgcccctgatggagcagggcaagcggtacgacgaggcctgcgc |
| | | cgagatctacggcgaccactacggcaagaagaacaccgaggagaagatctacctg |
| | | ccccccatccccgccgacgagatccggaaccccgtggtgctgcgggccctgtccc |
| | | aggcccggaaggtgatcaacggcgtggtgcggcggtacggctcccccgcccggat |
| | | ccacatcgagaccgcccgggaggtgggcaagtccttcaaggaccggaaggagatc |
| | | gagaagcggcaggaggagaaccggaaggaccgggagaaggccgccgccaagttcc |
| | | gggagtacttccccaacttcgtgggcgagcccaagtccaaggacatcctgaagct |
| | | gcggctgtacgagcagcagcacggcaagtgcctgtactccggcaaggagatcaac |
| | | ctggtgcggctgaacgagaagggctacgtggagatcgaccacgccctgcccttct |
| | | cccggacctgggacgactccttcaacaacaaggtgctggtgctgggctccgagaa |
| | | ccagaacaagggcaaccagaccccctacgagtacttcaacggcaaggacaactcc |
| | | cgggagtggcaggagttcaaggcccgggtggagacctcccggttcccccggtcca |
| | | agaagcagcggatcctgctgcagaagttcgacgaggacggcttcaaggagtgcaa |
| | | cctgaacgacacccggtacgtgaaccggttcctgtgccagttcgtggccgaccac |
| | | atcctgctgaccggcaagggcaagcggcgggtgttcgcctccaacggccagatca |
| | | ccaacctgctgcggggcttctggggcctgcggaaggtgcgggccgagaacgaccg |
| | | gcaccacgccctggacgccgtggtggtggcctgctccaccgtggccatgcagcag |
| | | aagatcacccggttcgtgcggtacaaggagatgaacgccttcgacggcaagacca |
| | | tcgacaaggagaccggcaaggtgctgcaccagaagacccacttcccccagccctg |
| | | ggagttcttcgcccaggaggtgatgatccgggtgttcggcaagcccgacggcaag |
| | | cccgagttcgaggaggccgacacccccgagaagctgcggaccctgctggccgaga |
| | | agctgtcctcccggcccgaggccgtgcacgagtacgtgacccccctgttcgtgtc |
| | | ccgggcccccaaccggaagatgtccggcgcccacaaggacaccctgcggtccgcc |
| | | aagcggttcgtgaagcacaacgagaagatctccgtgaagcgggtgtggctgaccg |
| | | agatcaagctggccgacctggagaacatggtgaactacaagaacggccgggagat |
| | | cgagctgtacgaggccctgaaggcccggctggaggcctacggcggcaacgccaag |
| | | caggccttcgaccccaaggacaaccccttctacaagaagggcggccagctggtga |
| | | aggccgtgcgggtggagaagacccaggagtccggcgtgctgctgaacaagaagaa |
| | | cgcctacaccatcgccgacaacggcgacatggtgcgggtggacgtgttctgcaag |
| | | gtggacaagaagggcaagaaccagtacttcatcgtgcccatctacgcctggcagg |
| | | tggccgagaacatcctgcccgacatcgactgcaagggctaccggatcgacgactc |
| | | ctacaccttctgcttctccctgcacaagtacgacctgatcgccttccagaaggac |
| | | gagaagtccaaggtggagttcgcctactacatcaactgcgactcctccaacggcc |
| | | ggttctacctggcctggcacgacaagggctccaaggagcagcagttccggatctc |
| | | cacccagaacctggtgctgatccagaagtaccaggtgaacgagctgggcaaggag |
| | | atccggccctgccggctgaagaagcggccccccgtgcggtag |
| 657 | Open reading | ATGGACGGCTCCGGCGGCGGCTCCCCCAAGAAGAAGCGGAAGGTGGAGGACAAGC |
| | frame for | GGCCCGCCGCCACCAAGAAGGCCGGCCAGGCCAAGAAGAAGAAGGGCGGCTCCGG |
| | Nme2Cas9 base | CGGCGGCGAGGCCTCCCCCGCCTCCGGCCCCCGGCACCTGATGGACCCCCACATC |
| | editor encoded | TTCACCTCCAACTTCAACAACGGCATCGGCCGGCACAAGACCTACCTGTGCTACG |
| | by mRNA R | AGGTGGAGCGGCTGGACAACGGCACCTCCGTGAAGATGGACCAGCACCGGGGCTT |
| | | CCTGCACAACCAGGCCAAGAACCTGCTGTGCGGCTTCTACGGCCGGCACGCCGAG |
| | | CTGCGGTTCCTGGACCTGGTGCCCTCCCTGCAGCTGGACCCCGCCCAGATCTACC |
| | | GGGTGACCTGGTTCATCTCCTGGTCCCCCTGCTTCTCCTGGGGCTGCGCCGGCGA |
| | | GGTGCGGGCCTTCCTGCAGGAGAACACCCACGTGCGGCTGCGGATCTTCGCCGCC |
| | | CGGATCTACGACTACGACCCCCTGTACAAGGAGGCCCTGCAGATGCTGCGGGACG |
| | | CCGGCGCCCAGGTGTCCATCATGACCTACGACGAGTTCAAGCACTGCTGGGACAC |
| | | CTTCGTGGACCACCAGGGCTGCCCCTTCCAGCCCTGGGACGGCCTGGACGAGCAC |
| | | TCCCAGGCCCTGTCCGGCCGGCTGCGGGCCATCCTGCAGAACCAGGGCAACTCCG |
| | | GCTCCGAGACCCCCGGCACCTCCGAGTCCGCCACCCCCGAGTCCGCAGCGTTCAA |
| | | ACCAAATcccatcaactacatcctgggcctggccatcggcatcgcctccgtgggc |
| | | tgggccatggtggagatcgacgaggaggagaaccccatccggctgatcgacctgg |
| | | gcgtgcgggtgttcgagcgggccgaggtgcccaagaccggcgactccctggccat |
| | | ggcccggcggctggcccggtccgtgcggcggctgacccggcggcgggcccaccgg |
| | | ctgctgcgggcccggcggctgctgaagcgggagggcgtgctgcaggccgccgact |
| | | tcgacgagaacggcctgatcaagtccctgcccaacaccccctggcagctgcgggc |
| | | cgccgccctggaccggaagctgacccccctggagtggtccgccgtgctgctgcac |
| | | ctgatcaagcaccggggctacctgtcccagcggaagaacgagggcgagaccgccg |
| | | acaaggagctgggcgccctgctgaagggcgtggccaacaacgcccacgccctgca |
| | | gaccggcgacttccggacccccgccgagctggccctgaacaagttcgagaaggag |
| | | tccggccacatccggaaccagcggggcgactactcccacaccttctcccggaagg |
| | | acctgcaggccgagctgatcctgctgttcgagaagcagaaggagttcggcaaccc |
| | | ccacgtgtccggcggcctgaaggagggcatcgagaccctgctgatgacccagcgg |
| | | cccgccctgtccggcgacgccgtgcagaagatgctgggccactgcaccttcgagc |
| | | ccgccgagcccaaggccgccaagaacacctacaccgccgagcggttcatctggct |
| | | gaccaagctgaacaacctgcggatcctggagcagggctccgagcggcccctgacc |
| | | gacaccgagcgggccaccctgatggacgagccctaccggaagtccaagctgacct |
| | | acgcccaggcccggaagctgctgggcctggaggacaccgccttcttcaagggcct |
| | | gcggtacggcaaggacaacgccgaggcctccaccctgatggagatgaaggcctac |
| | | cacgccatctcccgggccctggagaaggagggcctgaaggacaagaagtcccccc |
| | | tgaacctgtcctccgagctgcaggacgagatcggcaccgccttctccctgttcaa |
| | | gaccgacgaggacatcaccggccggctgaaggaccgggtgcagcccgagatcctg |
| | | gaggccctgctgaagcacatctccttcgacaagttcgtgcagatctccctgaagg |
| | | ccctgcggcggatcgtgcccctgatggagcagggcaagcggtacgacgaggcctg |
| | | cgccgagatctacggcgaccactacggcaagaagaacaccgaggagaagatctac |
| | | ctgccccccatccccgccgacgagatccggaaccccgtggtgctgcgggccctgt |
| | | cccaggcccggaaggtgatcaacggcgtggtgcggcggtacggctcccccgcccg |
| | | gatccacatcgagaccgcccgggaggtgggcaagtccttcaaggaccggaaggag |
| | | atcgagaagcggcaggaggagaaccggaaggaccgggagaaggccgccgccaagt |
| | | tccgggagtacttccccaacttcgtgggcgagcccaagtccaaggacatcctgaa |
| | | gctgcggctgtacgagcagcagcacggcaagtgcctgtactccggcaaggagatc |
| | | aacctggtgcggctgaacgagaagggctacgtggagatcgaccacgccctgccct |
| | | tctcccggacctgggacgactccttcaacaacaaggtgctggtgctgggctccga |
| | | gaaccagaacaagggcaaccagaccccctacgagtacttcaacggcaaggacaac |
| | | tcccgggagtggcaggagttcaaggcccgggtggagacctcccggttcccccggt |
| | | ccaagaagcagcggatcctgctgcagaagttcgacgaggacggcttcaaggagtg |
| | | caacctgaacgacacccggtacgtgaaccgcttcctgtgccagttcgtggccgac |
| | | cacatcctgctgaccggcaagggcaagcggcgggtgttcgcctccaacggccaga |
| | | tcaccaacctgctgcggggcttctggggcctgcggaaggtgcgggccgagaacga |
| | | ccggcaccacgccctggacgccgtggtggtggcctgctccaccgtggccatgcag |
| | | cagaagatcacccggttcgtgcggtacaaggagatgaacgccttcgacggcaaga |
| | | ccatcgacaaggagaccggcaaggtgctgcaccagaagacccacttcccccagcc |
| | | ctgggagttcttcgcccaggaggtgatgatccgggtgttcggcaagcccgacggc |
| | | aagcccgagttcgaggaggccgacacccccgagaagctgcggaccctgctggccg |
| | | agaagctgtcctcccggcccgaggccgtgcacgagtacgtgacccccctgttcgt |
| | | gtcccgggcccccaaccggaagatgtccggcgcccacaaggacaccctgcggtcc |
| | | gccaagcggttcgtgaagcacaacgagaagatctccgtgaagcgggtgtggctga |
| | | ccgagatcaagctggccgacctggagaacatggtgaactacaagaacggccggga |
| | | gatcgagctgtacgaggccctgaaggcccggctggaggcctacggcggcaacgcc |
| | | aagcaggccttcgaccccaaggacaaccccttctacaagaagggcggccagctgg |
| | | tgaaggccgtgcgggtggagaagacccaggagtccggcgtgctgctgaacaagaa |
| | | gaacgcctacaccatcgccgacaacggcgacatggtgcgggtggacgtgttctgc |
| | | aaggtggacaagaagggcaagaaccagtacttcatcgtgcccatctacgcctggc |
| | | aggtggccgagaacatcctgcccgacatcgactgcaagggctaccggatcgacga |
| | | ctcctacaccttctgcttctccctgcacaagtacgacctgatcgccttccagaag |
| | | gacgagaagtccaaggtggagttcgcctactacatcaactgcgactcctccaacg |
| | | gccggttctacctggcctggcacgacaagggctccaaggagcagcagttccggat |
| | | ctccacccagaacctggtgctgatccagaagtaccaggtgaacgagctgggcaag |
| | | gagatccggccctgccggctgaagaagcggccccccgtgcggtccggaaagcgga |
| | | ccgccgacggctccgagttcgagtcccccaagaagaagcggaaggtggagtag |
| 658 | Open reading | ATGGACGGCTCCGGCGGCGGCTCCCCCAAGAAGAAGCGGAAGGTGGAGGACAAGC |
| | frame for | GGCCCGCCGCCACCAAGAAGGCCGGCCAGGCCAAGAAGAAGAAGGGCGGCTCCGG |
| | Nme2Cas9 base | CGGCGGCGAGGCCTCCCCCGCCTCCGGCCCCCGGCACCTGATGGACCCCCACATC |
| | editor encoded | TTCACCTCCAACTTCAACAACGGCATCGGCCGGCACAAGACCTACCTGTGCTACG |
| | by mRNA R | AGGTGGAGCGGCTGGACAACGGCACCTCCGTGAAGATGGACCAGCACCGGGGCTT |
| | | CCTGCACAACCAGGCCAAGAACCTGCTGTGCGGCTTCTACGGCCGGCACGCCGAG |
| | | CTGCGGTTCCTGGACCTGGTGCCCTCCCTGCAGCTGGACCCCGCCCAGATCTACC |
| | | GGGTGACCTGGTTCATCTCCTGGTCCCCCTGCTTCTCCTGGGGCTGCGCCGGCGA |
| | | GGTGCGGGCCTTCCTGCAGGAGAACACCCACGTGCGGCTGCGGATCTTCGCCGCC |
| | | CGGATCTACGACTACGACCCCCTGTACAAGGAGGCCCTGCAGATGCTGCGGGACG |
| | | CCGGCGCCCAGGTGTCCATCATGACCTACGACGAGTTCAAGCACTGCTGGGACAC |
| | | CTTCGTGGACCACCAGGGCTGCCCCTTCCAGCCCTGGGACGGCCTGGACGAGCAC |
| | | TCCCAGGCCCTGTCCGGCCGGCTGCGGGCCATCCTGCAGAACCAGGGCAACTCCG |
| | | GCTCCGAGACCCCCGGCACCTCCGAGTCCGCCACCCCCGAGTCCGCAGCGTTCAA |
| | | ACCAAATcccatcaactacatcctgggcctggccatcggcatcgcctccgtgggc |
| | | tgggccatggtggagatcgacgaggaggagaaccccatccggctgatcgacctgg |
| | | gcgtgcgggtgttcgagcgggccgaggtgcccaagaccggcgactccctggccat |
| | | ggcccggcggctggcccggtccgtgcggcggctgacccggcggcgggcccaccgg |
| | | ctgctgcgggcccggcggctgctgaagcgggagggcgtgctgcaggccgccgact |
| | | tcgacgagaacggcctgatcaagtccctgcccaacaccccctggcagctgcgggc |
| | | cgccgccctggaccggaagctgacccccctggagtggtccgccgtgctgctgcac |
| | | ctgatcaagcaccggggctacctgtcccagcggaagaacgagggcgagaccgccg |
| | | acaaggagctgggcgccctgctgaagggcgtggccaacaacgcccacgccctgca |
| | | gaccggcgacttccggacccccgccgagctggccctgaacaagttcgagaaggag |
| | | tccggccacatccggaaccagcggggcgactactcccacaccttctcccggaagg |
| | | acctgcaggccgagctgatcctgctgttcgagaagcagaaggagttcggcaaccc |
| | | ccacgtgtccggcggcctgaaggagggcatcgagaccctgctgatgacccagcgg |
| | | cccgccctgtccggcgacgccgtgcagaagatgctgggccactgcaccttcgagc |
| | | ccgccgagcccaaggccgccaagaacacctacaccgccgagcggttcatctggct |
| | | gaccaagctgaacaacctgcggatcctggagcagggctccgagcggcccctgacc |
| | | gacaccgagcgggccaccctgatggacgagccctaccggaagtccaagctgacct |
| | | acgcccaggcccggaagctgctgggcctggaggacaccgccttcttcaagggcct |
| | | gcggtacggcaaggacaacgccgaggcctccaccctgatggagatgaaggcctac |
| | | cacgccatctcccgggccctggagaaggagggcctgaaggacaagaagtcccccc |
| | | tgaacctgtcctccgagctgcaggacgagatcggcaccgccttctccctgttcaa |
| | | gaccgacgaggacatcaccggccggctgaaggaccgggtgcagcccgagatcctg |
| | | gaggccctgctgaagcacatctccttcgacaagttcgtgcagatctccctgaagg |
| | | ccctgcggcggatcgtgcccctgatggagcagggcaagcggtacgacgaggcctg |
| | | cgccgagatctacggcgaccactacggcaagaagaacaccgaggagaagatctac |
| | | ctgccccccatccccgccgacgagatccggaaccccgtggtgctgcgggccctgt |
| | | cccaggcccggaaggtgatcaacggcgtggtgcggcggtacggctcccccgcccg |
| | | gatccacatcgagaccgcccgggaggtgggcaagtccttcaaggaccggaaggag |
| | | atcgagaagcggcaggaggagaaccggaaggaccgggagaaggccgccgccaagt |
| | | tccgggagtacttccccaacttcgtgggcgagcccaagtccaaggacatcctgaa |
| | | gctgcggctgtacgagcagcagcacggcaagtgcctgtactccggcaaggagatc |
| | | aacctggtgcggctgaacgagaagggctacgtggagatcgaccacgccctgccct |
| | | tctcccggacctgggacgactccttcaacaacaaggtgctggtgctgggctccga |
| | | gaaccagaacaagggcaaccagaccccctacgagtacttcaacggcaaggacaac |
| | | tcccgggagtggcaggagttcaaggcccgggtggagacctcccggttcccccggt |
| | | ccaagaagcagcggatcctgctgcagaagttcgacgaggacggcttcaaggagtg |
| | | caacctgaacgacacccggtacgtgaaccgcttcctgtgccagttcgtggccgac |
| | | cacatcctgctgaccggcaagggcaagcggcgggtgttcgcctccaacggccaga |
| | | tcaccaacctgctgcggggcttctggggcctgcggaaggtgcgggccgagaacga |
| | | ccggcaccacgccctggacgccgtggtggtggcctgctccaccgtggccatgcag |
| | | cagaagatcacccggttcgtgcggtacaaggagatgaacgccttcgacggcaaga |
| | | ccatcgacaaggagaccggcaaggtgctgcaccagaagacccacttcccccagcc |
| | | ctgggagttcttcgcccaggaggtgatgatccgggtgttcggcaagcccgacggc |
| | | aagcccgagttcgaggaggccgacacccccgagaagctgcggaccctgctggccg |
| | | agaagctgtcctcccggcccgaggccgtgcacgagtacgtgacccccctgttcgt |
| | | gtcccgggcccccaaccggaagatgtccggcgcccacaaggacaccctgcggtcc |
| | | gccaagcggttcgtgaagcacaacgagaagatctccgtgaagcgggtgtggctga |
| | | ccgagatcaagctggccgacctggagaacatggtgaactacaagaacggccggga |
| | | gatcgagctgtacgaggccctgaaggcccggctggaggcctacggcggcaacgcc |
| | | aagcaggccttcgaccccaaggacaaccccttctacaagaagggcggccagctgg |
| | | tgaaggccgtgcgggtggagaagacccaggagtccggcgtgctgctgaacaagaa |
| | | gaacgcctacaccatcgccgacaacggcgacatggtgcgggtggacgtgttctgc |
| | | aaggtggacaagaagggcaagaaccagtacttcatcgtgcccatctacgcctggc |
| | | aggtggccgagaacatcctgcccgacatcgactgcaagggctaccggatcgacga |
| | | ctcctacaccttctgcttctccctgcacaagtacgacctgatcgccttccagaag |
| | | gacgagaagtccaaggtggagttcgcctactacatcaactgcgactcctccaacg |
| | | gccggttctacctggcctggcacgacaagggctccaaggagcagcagttccggat |
| | | ctccacccagaacctggtgctgatccagaagtaccaggtgaacgagctgggcaag |
| | | gagatccggccctgccggctgaagaagcggccccccgtgcggtag |
| 659 | Open reading | ATGGAGGCCTCCCCCGCCTCCGGCCCCCGGCACCTGATGGACCCCCACATCTTCA |
| | frame for | CCTCCAACTTCAACAACGGCATCGGCCGGCACAAGACCTACCTGTGCTACGAGGT |
| | Nme2Cas9 base | GGAGCGGCTGGACAACGGCACCTCCGTGAAGATGGACCAGCACCGGGGCTTCCTG |
| | editor encoded | CACAACCAGGCCAAGAACCTGCTGTGCGGCTTCTACGGCCGGCACGCCGAGCTGC |
| | by mRNA S | GGTTCCTGGACCTGGTGCCCTCCCTGCAGCTGGACCCCGCCCAGATCTACCGGGT |
| | | GACCTGGTTCATCTCCTGGTCCCCCTGCTTCTCCTGGGGCTGCGCCGGCGAGGTG |
| | | CGGGCCTTCCTGCAGGAGAACACCCACGTGCGGCTGCGGATCTTCGCCGCCCGGA |
| | | TCTACGACTACGACCCCCTGTACAAGGAGGCCCTGCAGATGCTGCGGGACGCCGG |
| | | CGCCCAGGTGTCCATCATGACCTACGACGAGTTCAAGCACTGCTGGGACACCTTC |
| | | GTGGACCACCAGGGCTGCCCCTTCCAGCCCTGGGACGGCCTGGACGAGCACTCCC |
| | | AGGCCCTGTCCGGCCGGCTGCGGGCCATCCTGCAGAACCAGGGCAACTCCGGCTC |
| | | CGAGACCCCCGGCACCTCCGAGTCCGCCACCCCCGAGTCCGCAGCGTTCAAACCA |
| | | AATcccatcaactacatcctgggcctggccatcggcatcgcctccgtgggctggg |
| | | ccatggtggagatcgacgaggaggagaaccccatccggctgatcgacctgggcgt |
| | | gcgggtgttcgagcgggccgaggtgcccaagaccggcgactccctggccatggcc |
| | | cggcggctggcccggtccgtgcggcggctgacccggcggcgggcccaccggctgc |
| | | tgcgggcccggcggctgctgaagcgggagggcgtgctgcaggccgccgacttcga |
| | | cgagaacggcctgatcaagtccctgcccaacaccccctggcagctgcgggccgcc |
| | | gccctggaccggaagctgacccccctggagtggtccgccgtgctgctgcacctga |
| | | tcaagcaccggggctacctgtcccagcggaagaacgagggcgagaccgccgacaa |
| | | ggagctgggcgccctgctgaagggcgtggccaacaacgcccacgccctgcagacc |
| | | ggcgacttccggacccccgccgagctggccctgaacaagttcgagaaggagtccg |
| | | gccacatccggaaccagcggggcgactactcccacaccttctcccggaaggacct |
| | | gcaggccgagctgatcctgctgttcgagaagcagaaggagttcggcaacccccac |
| | | gtgtccggcggcctgaaggagggcatcgagaccctgctgatgacccagcggcccg |
| | | ccctgtccggcgacgccgtgcagaagatgctgggccactgcaccttcgagcccgc |
| | | cgagcccaaggccgccaagaacacctacaccgccgagcggttcatctggctgacc |
| | | aagctgaacaacctgcggatcctggagcagggctccgagcggcccctgaccgaca |
| | | ccgagcgggccaccctgatggacgagccctaccggaagtccaagctgacctacgc |
| | | ccaggcccggaagctgctgggcctggaggacaccgccttcttcaagggcctgcgg |
| | | tacggcaaggacaacgccgaggcctccaccctgatggagatgaaggcctaccacg |
| | | ccatctcccgggccctggagaaggagggcctgaaggacaagaagtcccccctgaa |
| | | cctgtcctccgagctgcaggacgagatcggcaccgccttctccctgttcaagacc |
| | | gacgaggacatcaccggccggctgaaggaccgggtgcagcccgagatcctggagg |
| | | ccctgctgaagcacatctccttcgacaagttcgtgcagatctccctgaaggccct |
| | | gcggcggatcgtgcccctgatggagcagggcaagcggtacgacgaggcctgcgcc |
| | | gagatctacggcgaccactacggcaagaagaacaccgaggagaagatctacctgc |
| | | cccccatccccgccgacgagatccggaaccccgtggtgctgcgggccctgtccca |
| | | ggcccggaaggtgatcaacggcgtggtgcggcggtacggctcccccgcccggatc |
| | | cacatcgagaccgcccgggaggtgggcaagtccttcaaggaccggaaggagatcg |
| | | agaagcggcaggaggagaaccggaaggaccgggagaaggccgccgccaagttccg |
| | | ggagtacttccccaacttcgtgggcgagcccaagtccaaggacatcctgaagctg |
| | | cggctgtacgagcagcagcacggcaagtgcctgtactccggcaaggagatcaacc |
| | | tggtgcggctgaacgagaagggctacgtggagatcgaccacgccctgcccttctc |
| | | ccggacctgggacgactccttcaacaacaaggtgctggtgctgggctccgagaac |
| | | cagaacaagggcaaccagaccccctacgagtacttcaacggcaaggacaactccc |
| | | gggagtggcaggagttcaaggcccgggtggagacctcccggttcccccggtccaa |
| | | gaagcagcggatcctgctgcagaagttcgacgaggacggcttcaaggagtgcaac |
| | | ctgaacgacacccggtacgtgaaccgcttcctgtgccagttcgtggccgaccaca |
| | | tcctgctgaccggcaagggcaagcggcgggtgttcgcctccaacggccagatcac |
| | | caacctgctgcggggcttctggggcctgcggaaggtgcgggccgagaacgaccgg |
| | | caccacgccctggacgccgtggtggtggcctgctccaccgtggccatgcagcaga |
| | | agatcacccggttcgtgcggtacaaggagatgaacgccttcgacggcaagaccat |
| | | cgacaaggagaccggcaaggtgctgcaccagaagacccacttcccccagccctgg |
| | | gagttcttcgcccaggaggtgatgatccgggtgttcggcaagcccgacggcaagc |
| | | ccgagttcgaggaggccgacacccccgagaagctgcggaccctgctggccgagaa |
| | | gctgtcctcccggcccgaggccgtgcacgagtacgtgacccccctgttcgtgtcc |
| | | cgggcccccaaccggaagatgtccggcgcccacaaggacaccctgcggtccgcca |
| | | agcggttcgtgaagcacaacgagaagatctccgtgaagcgggtgtggctgaccga |
| | | gatcaagctggccgacctggagaacatggtgaactacaagaacggccgggagatc |
| | | gagctgtacgaggccctgaaggcccggctggaggcctacggcggcaacgccaagc |
| | | aggccttcgaccccaaggacaaccccttctacaagaagggcggccagctggtgaa |
| | | ggccgtgcgggtggagaagacccaggagtccggcgtgctgctgaacaagaagaac |
| | | gcctacaccatcgccgacaacggcgacatggtgcgggtggacgtgttctgcaagg |
| | | tggacaagaagggcaagaaccagtacttcatcgtgcccatctacgcctggcaggt |
| | | ggccgagaacatcctgcccgacatcgactgcaagggctaccggatcgacgactcc |
| | | tacaccttctgcttctccctgcacaagtacgacctgatcgccttccagaaggacg |
| | | agaagtccaaggtggagttcgcctactacatcaactgcgactcctccaacggccg |
| | | gttctacctggcctggcacgacaagggctccaaggagcagcagttccggatctcc |
| | | acccagaacctggtgctgatccagaagtaccaggtgaacgagctgggcaaggaga |
| | | tccggccctgccggctgaagaagcggccccccgtgcggtccggaaagcggaccgc |
| | | cgacggctccgagttcgagtcccccaagaagaagcggaaggtggagtag |
| 660 | Open reading | ATGaagCTGggcTCCatcGAGttcATCaagGTGaacAAGggcTCCggcTCCggcT |
| | frame for | CCGGCgccCCCgagTCCgccACCgagTCCggcGGCaccTCCaccGAGtccGAGgg |
| | Nme2Cas9 | cTCCgccGGCaccTCCaccGAGtccGAGggctccGCCggcTCCgccGGCtccacc |
| | encoded | TCCaccGAGgagGGCaccTCCaccGAGtccGAGggctccGCCggcACCtccACCg |
| | by mRNA U | agtccgagGGCtccGCCggcACCtccGAGtccgccACCgagTCCggcGGCaccTC |
| | | CaccGAGtccGAGggcTCCtccTCCaccggtgccgccttcaagcccaaccccatc |
| | | aactacatcctgggcctggacatcggcatcgcctccgtgggctgggccatggtgg |
| | | agatcgacgaggaggagaaccccatccggctgatcgacctgggcgtgcgggtgtt |
| | | cgagcgggccgaggtgcccaagaccggcgactccctggccatggcccggcggctg |
| | | gcccggtccgtgcggcggctgacccggcggcgggcccaccggctgctgcgggccc |
| | | ggcggctgctgaagcgggagggcgtgctgcaggccgccgacttcgacgagaacgg |
| | | cctgatcaagtccctgcccaacaccccctggcagctgcgggccgccgccctggac |
| | | cggaagctgacccccctggagtggtccgccgtgctgctgcacctgatcaagcacc |
| | | ggggctacctgtcccagcggaagaacgagggcgagaccgccgacaaggagctggg |
| | | cgccctgctgaagggcgtggccaacaacgcccacgccctgcagaccggcgacttc |
| | | cggacccccgccgagctggccctgaacaagttcgagaaggagtccggccacatcc |
| | | ggaaccagcggggcgactactcccacaccttctcccggaaggacctgcaggccga |
| | | gctgatcctgctgttcgagaagcagaaggagttcggcaacccccacgtgtccggc |
| | | ggcctgaaggagggcatcgagaccctgctgatgacccagcggcccgccctgtccg |
| | | gcgacgccgtgcagaagatgctgggccactgcaccttcgagcccgccgagcccaa |
| | | ggccgccaagaacacctacaccgccgagcggttcatctggctgaccaagctgaac |
| | | aacctgcggatcctggagcagggctccgagcggcccctgaccgacaccgagcggg |
| | | ccaccctgatggacgagccctaccggaagtccaagctgacctacgcccaggcccg |
| | | gaagctgctgggcctggaggacaccgccttcttcaagggcctgcggtacggcaag |
| | | gacaacgccgaggcctccaccctgatggagatgaaggcctaccacgccatctccc |
| | | gggccctggagaaggagggcctgaaggacaagaagtcccccctgaacctgtcctc |
| | | cgagctgcaggacgagatcggcaccgccttctccctgttcaagaccgacgaggac |
| | | atcaccggccggctgaaggaccgggtgcagcccgagatcctggaggccctgctga |
| | | agcacatctccttcgacaagttcgtgcagatctccctgaaggccctgcggcggat |
| | | cgtgcccctgatggagcagggcaagcggtacgacgaggcctgcgccgagatctac |
| | | ggcgaccactacggcaagaagaacaccgaggagaagatctacctgccccccatcc |
| | | ccgccgacgagatccggaaccccgtggtgctgcgggccctgtcccaggcccggaa |
| | | ggtgatcaacggcgtggtgcggcggtacggctcccccgcccggatccacatcgag |
| | | accgcccgggaggtgggcaagtccttcaaggaccggaaggagatcgagaagcggc |
| | | aggaggagaaccggaaggaccgggagaaggccgccgccaagttccgggagtactt |
| | | ccccaacttcgtgggcgagcccaagtccaaggacatcctgaagctgcggctgtac |
| | | gagcagcagcacggcaagtgcctgtactccggcaaggagatcaacctggtgcggc |
| | | tgaacgagaagggctacgtggagatcgaccacgccctgcccttctcccggacctg |
| | | ggacgactccttcaacaacaaggtgctggtgctgggctccgagaaccagaacaag |
| | | ggcaaccagaccccctacgagtacttcaacggcaaggacaactcccgggagtggc |
| | | aggagttcaaggcccgggtggagacctcccggttcccccggtccaagaagcagcg |
| | | gatcctgctgcagaagttcgacgaggacggcttcaaggagtgcaacctgaacgac |
| | | acccggtacgtgaaccgcttcctgtgccagttcgtggccgaccacatcctgctga |
| | | ccggcaagggcaagcggcgggtgttcgcctccaacggccagatcaccaacctgct |
| | | gcggggcttctggggcctgcggaaggtgcgggccgagaacgaccggcaccacgcc |
| | | ctggacgccgtggtggtggcctgctccaccgtggccatgcagcagaagatcaccc |
| | | ggttcgtgcggtacaaggagatgaacgccttcgacggcaagaccatcgacaagga |
| | | gaccggcaaggtgctgcaccagaagacccacttcccccagccctgggagttcttc |
| | | gcccaggaggtgatgatccgggtgttcggcaagcccgacggcaagcccgagttcg |
| | | aggaggccgacacccccgagaagctgcggaccctgctggccgagaagctgtcctc |
| | | ccggcccgaggccgtgcacgagtacgtgacccccctgttcgtgtcccgggccccc |
| | | aaccggaagatgtccggcgcccacaaggacaccctgcggtccgccaagcggttcg |
| | | tgaagcacaacgagaagatctccgtgaagcgggtgtggctgaccgagatcaagct |
| | | ggccgacctggagaacatggtgaactacaagaacggccgggagatcgagctgtac |
| | | gaggccctgaaggcccggctggaggcctacggcggcaacgccaagcaggccttcg |
| | | accccaaggacaaccccttctacaagaagggcggccagctggtgaaggccgtgcg |
| | | ggtggagaagacccaggagtccggcgtgctgctgaacaagaagaacgcctacacc |
| | | atcgccgacaacggcgacatggtgcgggtggacgtgttctgcaaggtggacaaga |
| | | agggcaagaaccagtacttcatcgtgcccatctacgcctggcaggtggccgagaa |
| | | catcctgcccgacatcgactgcaagggctaccggatcgacgactcctacaccttc |
| | | tgcttctccctgcacaagtacgacctgatcgccttccagaaggacgagaagtcca |
| | | aggtggagttcgcctactacatcaactgcgactcctccaacggccggttctacct |
| | | ggcctggcacgacaagggctccaaggagcagcagttccggatctccacccagaac |
| | | ctggtgctgatccagaagtaccaggtgaacgagctgggcaaggagatccggccct |
| | | gccggctgaagaagcggccccccgtgcggtccggaaagcggaccgccgacggctc |
| | | cgagttcgagtcccccaagaagaagcggaaggtggagtga |
| 661 | ORF encoding Sp. | ATGGACAAGAAGTACAGCATCGGACTGGACATCGGAACAAACAGCGTCGGATGGG |
| | Cas9 | CAGTCATCACAGACGAATACAAGGTCCCGAGCAAGAAGTTCAAGGTCCTGGGAAA |
| | | CACAGACAGACACAGCATCAAGAAGAACCTGATCGGAGCACTGCTGTTCGACAGC |
| | | GGAGAAACAGCAGAAGCAACAAGACTGAAGAGAACAGCAAGAAGAAGATACACAA |
| | | GAAGAAAGAACAGAATCTGCTACCTGCAGGAAATCTTCAGCAACGAAATGGCAAA |
| | | GGTCGACGACAGCTTCTTCCACAGACTGGAAGAAAGCTTCCTGGTCGAAGAAGAC |
| | | AAGAAGCACGAAAGACACCCGATCTTCGGAAACATCGTCGACGAAGTCGCATACC |
| | | ACGAAAAGTACCCGACAATCTACCACCTGAGAAAGAAGCTGGTCGACAGCACAGA |
| | | CAAGGCAGACCTGAGACTGATCTACCTGGCACTGGCACACATGATCAAGTTCAGA |
| | | GGACACTTCCTGATCGAAGGAGACCTGAACCCGGACAACAGCGACGTCGACAAGC |
| | | TGTTCATCCAGCTGGTCCAGACATACAACCAGCTGTTCGAAGAAAACCCGATCAA |
| | | CGCAAGCGGAGTCGACGCAAAGGCAATCCTGAGCGCAAGACTGAGCAAGAGCAGA |
| | | AGACTGGAAAACCTGATCGCACAGCTGCCGGGAGAAAAGAAGAACGGACTGTTCG |
| | | GAAACCTGATCGCACTGAGCCTGGGACTGACACCGAACTTCAAGAGCAACTTCGA |
| | | CCTGGCAGAAGACGCAAAGCTGCAGCTGAGCAAGGACACATACGACGACGACCTG |
| | | GACAACCTGCTGGCACAGATCGGAGACCAGTACGCAGACCTGTTCCTGGCAGCAA |
| | | AGAACCTGAGCGACGCAATCCTGCTGAGCGACATCCTGAGAGTCAACACAGAAAT |
| | | CACAAAGGCACCGCTGAGCGCAAGCATGATCAAGAGATACGACGAACACCACCAG |
| | | GACCTGACACTGCTGAAGGCACTGGTCAGACAGCAGCTGCCGGAAAAGTACAAGG |
| | | AAATCTTCTTCGACCAGAGCAAGAACGGATACGCAGGATACATCGACGGAGGAGC |
| | | AAGCCAGGAAGAATTCTACAAGTTCATCAAGCCGATCCTGGAAAAGATGGACGGA |
| | | ACAGAAGAACTGCTGGTCAAGCTGAACAGAGAAGACCTGCTGAGAAAGCAGAGAA |
| | | CATTCGACAACGGAAGCATCCCGCACCAGATCCACCTGGGAGAACTGCACGCAAT |
| | | CCTGAGAAGACAGGAAGACTTCTACCCGTTCCTGAAGGACAACAGAGAAAAGATC |
| | | GAAAAGATCCTGACATTCAGAATCCCGTACTACGTCGGACCGCTGGCAAGAGGAA |
| | | ACAGCAGATTCGCATGGATGACAAGAAAGAGCGAAGAAACAATCACACCGTGGAA |
| | | CTTCGAAGAAGTCGTCGACAAGGGAGCAAGCGCACAGAGCTTCATCGAAAGAATG |
| | | ACAAACTTCGACAAGAACCTGCCGAACGAAAAGGTCCTGCCGAAGCACAGCCTGC |
| | | TGTACGAATACTTCACAGTCTACAACGAACTGACAAAGGTCAAGTACGTCACAGA |
| | | AGGAATGAGAAAGCCGGCATTCCTGAGCGGAGAACAGAAGAAGGCAATCGTCGAC |
| | | CTGCTGTTCAAGACAAACAGAAAGGTCACAGTCAAGCAGCTGAAGGAAGACTACT |
| | | TCAAGAAGATCGAATGCTTCGACAGCGTCGAAATCAGCGGAGTCGAAGACAGATT |
| | | CAACGCAAGCCTGGGAACATACCACGACCTGCTGAAGATCATCAAGGACAAGGAC |
| | | TTCCTGGACAACGAAGAAAACGAAGACATCCTGGAAGACATCGTCCTGACACTGA |
| | | CACTGTTCGAAGACAGAGAAATGATCGAAGAAAGACTGAAGACATACGCACACCT |
| | | GTTCGACGACAAGGTCATGAAGCAGCTGAAGAGAAGAAGATACACAGGATGGGGA |
| | | AGACTGAGCAGAAAGCTGATCAACGGAATCAGAGACAAGCAGAGCGGAAAGACAA |
| | | TCCTGGACTTCCTGAAGAGCGACGGATTCGCAAACAGAAACTTCATGCAGCTGAT |
| | | CCACGACGACAGCCTGACATTCAAGGAAGACATCCAGAAGGCACAGGTCAGCGGA |
| | | CAGGGAGACAGCCTGCACGAACACATCGCAAACCTGGCAGGAAGCCCGGCAATCA |
| | | AGAAGGGAATCCTGCAGACAGTCAAGGTCGTCGACGAACTGGTCAAGGTCATGGG |
| | | AAGACACAAGCCGGAAAACATCGTCATCGAAATGGCAAGAGAAAACCAGACAACA |
| | | CAGAAGGGACAGAAGAACAGCAGAGAAAGAATGAAGAGAATCGAAGAAGGAATCA |
| | | AGGAACTGGGAAGCCAGATCCTGAAGGAACACCCGGTCGAAAACACACAGCTGCA |
| | | GAACGAAAAGCTGTACCTGTACTACCTGCAGAACGGAAGAGACATGTACGTCGAC |
| | | CAGGAACTGGACATCAACAGACTGAGCGACTACGACGTCGACCACATCGTCCCGC |
| | | AGAGCTTCCTGAAGGACGACAGCATCGACAACAAGGTCCTGACAAGAAGCGACAA |
| | | GAACAGAGGAAAGAGCGACAACGTCCCGAGCGAAGAAGTCGTCAAGAAGATGAAG |
| | | AACTACTGGAGACAGCTGCTGAACGCAAAGCTGATCACACAGAGAAAGTTCGACA |
| | | ACCTGACAAAGGCAGAGAGAGGAGGACTGAGCGAACTGGACAAGGCAGGATTCAT |
| | | CAAGAGACAGCTGGTCGAAACAAGACAGATCACAAAGCACGTCGCACAGATCCTG |
| | | GACAGCAGAATGAACACAAAGTACGACGAAAACGACAAGCTGATCAGAGAAGTCA |
| | | AGGTCATCACACTGAAGAGCAAGCTGGTCAGCGACTTCAGAAAGGACTTCCAGTT |
| | | CTACAAGGTCAGAGAAATCAACAACTACCACCACGCACACGACGCATACCTGAAC |
| | | GCAGTCGTCGGAACAGCACTGATCAAGAAGTACCCGAAGCTGGAAAGCGAATTCG |
| | | TCTACGGAGACTACAAGGTCTACGACGTCAGAAAGATGATCGCAAAGAGCGAACA |
| | | GGAAATCGGAAAGGCAACAGCAAAGTACTTCTTCTACAGCAACATCATGAACTTC |
| | | TTCAAGACAGAAATCACACTGGCAAACGGAGAAATCAGAAAGAGACCGCTGATCG |
| | | AAACAAACGGAGAAACAGGAGAAATCGTCTGGGACAAGGGAAGAGACTTCGCAAC |
| | | AGTCAGAAAGGTCCTGAGCATGCCGCAGGTCAACATCGTCAAGAAGACAGAAGTC |
| | | CAGACAGGAGGATTCAGCAAGGAAAGCATCCTGCCGAAGAGAAACAGCGACAAGC |
| | | TGATCGCAAGAAAGAAGGACTGGGACCCGAAGAAGTACGGAGGATTCGACAGCCC |
| | | GACAGTCGCATACAGCGTCCTGGTCGTCGCAAAGGTCGAAAAGGGAAAGAGCAAG |
| | | AAGCTGAAGAGCGTCAAGGAACTGCTGGGAATCACAATCATGGAAAGAAGCAGCT |
| | | TCGAAAAGAACCCGATCGACTTCCTGGAAGCAAAGGGATACAAGGAAGTCAAGAA |
| | | GGACCTGATCATCAAGCTGCCGAAGTACAGCCTGTTCGAACTGGAAAACGGAAGA |
| | | AAGAGAATGCTGGCAAGCGCAGGAGAACTGCAGAAGGGAAACGAACTGGCACTGC |
| | | CGAGCAAGTACGTCAACTTCCTGTACCTGGCAAGCCACTACGAAAAGCTGAAGGG |
| | | AAGCCCGGAAGACAACGAACAGAAGCAGCTGTTCGTCGAACAGCACAAGCACTAC |
| | | CTGGACGAAATCATCGAACAGATCAGCGAATTCAGCAAGAGAGTCATCCTGGCAG |
| | | ACGCAAACCTGGACAAGGTCCTGAGCGCATACAACAAGCACAGAGACAAGCCGAT |
| | | CAGAGAACAGGCAGAAAACATCATCCACCTGTTCACACTGACAAACCTGGGAGCA |
| | | CCGGCAGCATTCAAGTACTTCGACACAACAATCGACAGAAAGAGATACACAAGCA |
| | | CAAAGGAAGTCCTGGACGCAACACTGATCCACCAGAGCATCACAGGACTGTACGA |
| | | AACAAGAATCGACCTGAGCCAGCTGGGAGGAGACGGAGGAGGAAGCCCGAAGAAG |
| | | AAGAGAAAGGTCTAG |
| 662 | ORF encoding Sp. | ATGGACAAGAAGTACTCCATCGGCCTGGACATCGGCACCAACTCCGTGGGCTGGG |
| | Cas9 | CCGTGATCACCGACGAGTACAAGGTGCCCTCCAAGAAGTTCAAGGTGCTGGGCAA |
| | | CACCGACCGGCACTCCATCAAGAAGAACCTGATCGGCGCCCTGCTGTTCGACTCC |
| | | GGCGAGACCGCCGAGGCCACCCGGCTGAAGCGGACCGCCCGGCGGCGGTACACCC |
| | | GGCGGAAGAACCGGATCTGCTACCTGCAGGAGATCTTCTCCAACGAGATGGCCAA |
| | | GGTGGACGACTCCTTCTTCCACCGGCTGGAGGAGTCCTTCCTGGTGGAGGAGGAC |
| | | AAGAAGCACGAGCGGCACCCCATCTTCGGCAACATCGTGGACGAGGTGGCCTACC |
| | | ACGAGAAGTACCCCACCATCTACCACCTGCGGAAGAAGCTGGTGGACTCCACCGA |
| | | CAAGGCCGACCTGCGGCTGATCTACCTGGCCCTGGCCCACATGATCAAGTTCCGG |
| | | GGCCACTTCCTGATCGAGGGCGACCTGAACCCCGACAACTCCGACGTGGACAAGC |
| | | TGTTCATCCAGCTGGTGCAGACCTACAACCAGCTGTTCGAGGAGAACCCCATCAA |
| | | CGCCTCCGGCGTGGACGCCAAGGCCATCCTGTCCGCCCGGCTGTCCAAGTCCCGG |
| | | CGGCTGGAGAACCTGATCGCCCAGCTGCCCGGCGAGAAGAAGAACGGCCTGTTCG |
| | | GCAACCTGATCGCCCTGTCCCTGGGCCTGACCCCCAACTTCAAGTCCAACTTCGA |
| | | CCTGGCCGAGGACGCCAAGCTGCAGCTGTCCAAGGACACCTACGACGACGACCTG |
| | | GACAACCTGCTGGCCCAGATCGGCGACCAGTACGCCGACCTGTTCCTGGCCGCCA |
| | | AGAACCTGTCCGACGCCATCCTGCTGTCCGACATCCTGCGGGTGAACACCGAGAT |
| | | CACCAAGGCCCCCCTGTCCGCCTCCATGATCAAGCGGTACGACGAGCACCACCAG |
| | | GACCTGACCCTGCTGAAGGCCCTGGTGCGGCAGCAGCTGCCCGAGAAGTACAAGG |
| | | AGATCTTCTTCGACCAGTCCAAGAACGGCTACGCCGGCTACATCGACGGCGGCGC |
| | | CTCCCAGGAGGAGTTCTACAAGTTCATCAAGCCCATCCTGGAGAAGATGGACGGC |
| | | ACCGAGGAGCTGCTGGTGAAGCTGAACCGGGAGGACCTGCTGCGGAAGCAGCGGA |
| | | CCTTCGACAACGGCTCCATCCCCCACCAGATCCACCTGGGCGAGCTGCACGCCAT |
| | | CCTGCGGCGGCAGGAGGACTTCTACCCCTTCCTGAAGGACAACCGGGAGAAGATC |
| | | GAGAAGATCCTGACCTTCCGGATCCCCTACTACGTGGGCCCCCTGGCCCGGGGCA |
| | | ACTCCCGGTTCGCCTGGATGACCCGGAAGTCCGAGGAGACCATCACCCCCTGGAA |
| | | CTTCGAGGAGGTGGTGGACAAGGGCGCCTCCGCCCAGTCCTTCATCGAGCGGATG |
| | | ACCAACTTCGACAAGAACCTGCCCAACGAGAAGGTGCTGCCCAAGCACTCCCTGC |
| | | TGTACGAGTACTTCACCGTGTACAACGAGCTGACCAAGGTGAAGTACGTGACCGA |
| | | GGGCATGCGGAAGCCCGCCTTCCTGTCCGGCGAGCAGAAGAAGGCCATCGTGGAC |
| | | CTGCTGTTCAAGACCAACCGGAAGGTGACCGTGAAGCAGCTGAAGGAGGACTACT |
| | | TCAAGAAGATCGAGTGCTTCGACTCCGTGGAGATCTCCGGCGTGGAGGACCGGTT |
| | | CAACGCCTCCCTGGGCACCTACCACGACCTGCTGAAGATCATCAAGGACAAGGAC |
| | | TTCCTGGACAACGAGGAGAACGAGGACATCCTGGAGGACATCGTGCTGACCCTGA |
| | | CCCTGTTCGAGGACCGGGAGATGATCGAGGAGCGGCTGAAGACCTACGCCCACCT |
| | | GTTCGACGACAAGGTGATGAAGCAGCTGAAGCGGCGGCGGTACACCGGCTGGGGC |
| | | CGGCTGTCCCGGAAGCTGATCAACGGCATCCGGGACAAGCAGTCCGGCAAGACCA |
| | | TCCTGGACTTCCTGAAGTCCGACGGCTTCGCCAACCGGAACTTCATGCAGCTGAT |
| | | CCACGACGACTCCCTGACCTTCAAGGAGGACATCCAGAAGGCCCAGGTGTCCGGC |
| | | CAGGGCGACTCCCTGCACGAGCACATCGCCAACCTGGCCGGCTCCCCCGCCATCA |
| | | AGAAGGGCATCCTGCAGACCGTGAAGGTGGTGGACGAGCTGGTGAAGGTGATGGG |
| | | CCGGCACAAGCCCGAGAACATCGTGATCGAGATGGCCCGGGAGAACCAGACCACC |
| | | CAGAAGGGCCAGAAGAACTCCCGGGAGCGGATGAAGCGGATCGAGGAGGGCATCA |
| | | AGGAGCTGGGCTCCCAGATCCTGAAGGAGCACCCCGTGGAGAACACCCAGCTGCA |
| | | GAACGAGAAGCTGTACCTGTACTACCTGCAGAACGGCCGGGACATGTACGTGGAC |
| | | CAGGAGCTGGACATCAACCGGCTGTCCGACTACGACGTGGACCACATCGTGCCCC |
| | | AGTCCTTCCTGAAGGACGACTCCATCGACAACAAGGTGCTGACCCGGTCCGACAA |
| | | GAACCGGGGCAAGTCCGACAACGTGCCCTCCGAGGAGGTGGTGAAGAAGATGAAG |
| | | AACTACTGGCGGCAGCTGCTGAACGCCAAGCTGATCACCCAGCGGAAGTTCGACA |
| | | ACCTGACCAAGGCCGAGCGGGGCGGCCTGTCCGAGCTGGACAAGGCCGGCTTCAT |
| | | CAAGCGGCAGCTGGTGGAGACCCGGCAGATCACCAAGCACGTGGCCCAGATCCTG |
| | | GACTCCCGGATGAACACCAAGTACGACGAGAACGACAAGCTGATCCGGGAGGTGA |
| | | AGGTGATCACCCTGAAGTCCAAGCTGGTGTCCGACTTCCGGAAGGACTTCCAGTT |
| | | CTACAAGGTGCGGGAGATCAACAACTACCACCACGCCCACGACGCCTACCTGAAC |
| | | GCCGTGGTGGGCACCGCCCTGATCAAGAAGTACCCCAAGCTGGAGTCCGAGTTCG |
| | | TGTACGGCGACTACAAGGTGTACGACGTGCGGAAGATGATCGCCAAGTCCGAGCA |
| | | GGAGATCGGCAAGGCCACCGCCAAGTACTTCTTCTACTCCAACATCATGAACTTC |
| | | TTCAAGACCGAGATCACCCTGGCCAACGGCGAGATCCGGAAGCGGCCCCTGATCG |
| | | AGACCAACGGCGAGACCGGCGAGATCGTGTGGGACAAGGGCCGGGACTTCGCCAC |
| | | CGTGCGGAAGGTGCTGTCCATGCCCCAGGTGAACATCGTGAAGAAGACCGAGGTG |
| | | CAGACCGGCGGCTTCTCCAAGGAGTCCATCCTGCCCAAGCGGAACTCCGACAAGC |
| | | TGATCGCCCGGAAGAAGGACTGGGACCCCAAGAAGTACGGCGGCTTCGACTCCCC |
| | | CACCGTGGCCTACTCCGTGCTGGTGGTGGCCAAGGTGGAGAAGGGCAAGTCCAAG |
| | | AAGCTGAAGTCCGTGAAGGAGCTGCTGGGCATCACCATCATGGAGCGGTCCTCCT |
| | | TCGAGAAGAACCCCATCGACTTCCTGGAGGCCAAGGGCTACAAGGAGGTGAAGAA |
| | | GGACCTGATCATCAAGCTGCCCAAGTACTCCCTGTTCGAGCTGGAGAACGGCCGG |
| | | AAGCGGATGCTGGCCTCCGCCGGCGAGCTGCAGAAGGGCAACGAGCTGGCCCTGC |
| | | CCTCCAAGTACGTGAACTTCCTGTACCTGGCCTCCCACTACGAGAAGCTGAAGGG |
| | | CTCCCCCGAGGACAACGAGCAGAAGCAGCTGTTCGTGGAGCAGCACAAGCACTAC |
| | | CTGGACGAGATCATCGAGCAGATCTCCGAGTTCTCCAAGCGGGTGATCCTGGCCG |
| | | ACGCCAACCTGGACAAGGTGCTGTCCGCCTACAACAAGCACCGGGACAAGCCCAT |
| | | CCGGGAGCAGGCCGAGAACATCATCCACCTGTTCACCCTGACCAACCTGGGCGCC |
| | | CCCGCCGCCTTCAAGTACTTCGACACCACCATCGACCGGAAGCGGTACACCTCCA |
| | | CCAAGGAGGTGCTGGACGCCACCCTGATCCACCAGTCCATCACCGGCCTGTACGA |
| | | GACCCGGATCGACCTGTCCCAGCTGGGCGGCGACGGCGGCGGCTCCCCCAAGAAG |
| | | AAGCGGAAGGTGTGA |
| 663 | ORF encoding Sp. | AUGGACAAGAAGUACAGCAUCGGCCUGGACAUCGGCACGAACAGCGUUGGCUGGG |
| | Cas9 | CUGUGAUCACGGACGAGUACAAGGUUCCCUCAAAGAAGUUCAAGGUGCUGGGCAA |
| | | CACGGACCGGCACAGCAUCAAGAAGAAUCUCAUCGGUGCACUGCUGUUCGACAGC |
| | | GGUGAGACGGCCGAAGCCACGCGGCUGAAGCGGACGGCCCGCCGGCGGUACACGC |
| | | GGCGGAAGAACCGGAUCUGCUACCUGCAGGAGAUCUUCAGCAACGAGAUGGCCAA |
| | | GGUGGACGACAGCUUCUUCCACCGGCUGGAGGAGAGCUUCCUGGUGGAGGAGGAC |
| | | AAGAAGCACGAGCGGCACCCCAUCUUCGGCAACAUCGUGGACGAAGUCGCCUACC |
| | | ACGAGAAGUACCCCACCAUCUACCACCUGCGGAAGAAGCUGGUGGACUCGACUGA |
| | | CAAGGCCGACCUGCGGCUGAUCUACCUGGCACUGGCCCACAUGAUAAAGUUCCGG |
| | | GGCCACUUCCUGAUCGAGGGCGACCUGAACCCUGACAACAGCGACGUGGACAAGC |
| | | UGUUCAUCCAGCUGGUGCAGACCUACAACCAGCUGUUCGAGGAGAACCCCAUCAA |
| | | CGCCAGCGGCGUGGACGCCAAGGCCAUCCUCAGCGCCCGCCUCAGCAAGAGCCGG |
| | | CGGCUGGAGAAUCUCAUCGCCCAGCUUCCAGGUGAGAAGAAGAAUGGGCUGUUCG |
| | | GCAAUCUCAUCGCACUCAGCCUGGGCCUGACUCCCAACUUCAAGAGCAACUUCGA |
| | | CCUGGCCGAGGACGCCAAGCUGCAGCUCAGCAAGGACACCUACGACGACGACCUG |
| | | GACAAUCUCCUGGCCCAGAUCGGCGACCAGUACGCCGACCUGUUCCUGGCUGCCA |
| | | AGAAUCUCAGCGACGCCAUCCUGCUCAGCGACAUCCUGCGGGUGAACACAGAGAU |
| | | CACGAAGGCCCCCCUCAGCGCCAGCAUGAUAAAGCGGUACGACGAGCACCACCAG |
| | | GACCUGACGCUGCUGAAGGCACUGGUGCGGCAGCAGCUUCCAGAGAAGUACAAGG |
| | | AGAUCUUCUUCGACCAGAGCAAGAAUGGGUACGCCGGGUACAUCGACGGUGGUGC |
| | | CAGCCAGGAGGAGUUCUACAAGUUCAUCAAGCCCAUCCUGGAGAAGAUGGACGGC |
| | | ACAGAGGAGCUGCUGGUGAAGCUGAACAGGGAGGACCUGCUGCGGAAGCAGCGGA |
| | | CGUUCGACAAUGGGAGCAUCCCCCACCAGAUCCACCUGGGUGAGCUGCACGCCAU |
| | | CCUGCGGCGGCAGGAGGACUUCUACCCCUUCCUGAAGGACAACAGGGAGAAGAUC |
| | | GAGAAGAUCCUGACGUUCCGGAUCCCCUACUACGUUGGCCCCCUGGCCCGCGGCA |
| | | ACAGCCGGUUCGCCUGGAUGACGCGGAAGAGCGAGGAGACGAUCACUCCCUGGAA |
| | | CUUCGAGGAAGUCGUGGACAAGGGUGCCAGCGCCCAGAGCUUCAUCGAGCGGAUG |
| | | ACGAACUUCGACAAGAAUCUUCCAAACGAGAAGGUGCUUCCAAAGCACAGCCUGC |
| | | UGUACGAGUACUUCACGGUGUACAACGAGCUGACGAAGGUGAAGUACGUGACAGA |
| | | GGGCAUGCGGAAGCCCGCCUUCCUCAGCGGUGAGCAGAAGAAGGCCAUCGUGGAC |
| | | CUGCUGUUCAAGACGAACCGGAAGGUGACGGUGAAGCAGCUGAAGGAGGACUACU |
| | | UCAAGAAGAUCGAGUGCUUCGACAGCGUGGAGAUCAGCGGCGUGGAGGACCGGUU |
| | | CAACGCCAGCCUGGGCACCUACCACGACCUGCUGAAGAUCAUCAAGGACAAGGAC |
| | | UUCCUGGACAACGAGGAGAACGAGGACAUCCUGGAGGACAUCGUGCUGACGCUGA |
| | | CGCUGUUCGAGGACAGGGAGAUGAUAGAGGAGCGGCUGAAGACCUACGCCCACCU |
| | | GUUCGACGACAAGGUGAUGAAGCAGCUGAAGCGGCGGCGGUACACGGGCUGGGGC |
| | | CGGCUCAGCCGGAAGCUGAUCAAUGGGAUCCGAGACAAGCAGAGCGGCAAGACGA |
| | | UCCUGGACUUCCUGAAGAGCGACGGCUUCGCCAACCGGAACUUCAUGCAGCUGAU |
| | | CCACGACGACAGCCUGACGUUCAAGGAGGACAUCCAGAAGGCCCAGGUCAGCGGC |
| | | CAGGGCGACAGCCUGCACGAGCACAUCGCCAAUCUCGCCGGGAGCCCCGCCAUCA |
| | | AGAAGGGGAUCCUGCAGACGGUGAAGGUGGUGGACGAGCUGGUGAAGGUGAUGGG |
| | | CCGGCACAAGCCAGAGAACAUCGUGAUCGAGAUGGCCAGGGAGAACCAGACGACU |
| | | CAAAAGGGGCAGAAGAACAGCAGGGAGCGGAUGAAGCGGAUCGAGGAGGGCAUCA |
| | | AGGAGCUGGGCAGCCAGAUCCUGAAGGAGCACCCCGUGGAGAACACUCAACUGCA |
| | | GAACGAGAAGCUGUACCUGUACUACCUGCAGAAUGGGCGAGACAUGUACGUGGAC |
| | | CAGGAGCUGGACAUCAACCGGCUCAGCGACUACGACGUGGACCACAUCGUUCCCC |
| | | AGAGCUUCCUGAAGGACGACAGCAUCGACAACAAGGUGCUGACGCGGAGCGACAA |
| | | GAACCGGGGCAAGAGCGACAACGUUCCCUCAGAGGAAGUCGUGAAGAAGAUGAAG |
| | | AACUACUGGCGGCAGCUGCUGAACGCCAAGCUGAUCACUCAACGGAAGUUCGACA |
| | | AUCUCACGAAGGCCGAGCGGGGUGGCCUCAGCGAGCUGGACAAGGCCGGGUUCAU |
| | | CAAGCGGCAGCUGGUGGAGACGCGGCAGAUCACGAAGCACGUGGCCCAGAUCCUG |
| | | GACAGCCGGAUGAACACGAAGUACGACGAGAACGACAAGCUGAUCAGGGAAGUCA |
| | | AGGUGAUCACGCUGAAGAGCAAGCUGGUCAGCGACUUCCGGAAGGACUUCCAGUU |
| | | CUACAAGGUGAGGGAGAUCAACAACUACCACCACGCCCACGACGCCUACCUGAAC |
| | | GCUGUGGUUGGCACGGCACUGAUCAAGAAGUACCCCAAGCUGGAGAGCGAGUUCG |
| | | UGUACGGCGACUACAAGGUGUACGACGUGCGGAAGAUGAUAGCCAAGAGCGAGCA |
| | | GGAGAUCGGCAAGGCCACGGCCAAGUACUUCUUCUACAGCAACAUCAUGAACUUC |
| | | UUCAAGACAGAGAUCACGCUGGCCAAUGGUGAGAUCCGGAAGCGGCCCCUGAUCG |
| | | AGACGAAUGGUGAGACGGGUGAGAUCGUGUGGGACAAGGGGCGAGACUUCGCCAC |
| | | GGUGCGGAAGGUGCUCAGCAUGCCCCAGGUGAACAUCGUGAAGAAGACAGAAGUC |
| | | CAGACGGGUGGCUUCAGCAAGGAGAGCAUCCUUCCAAAGCGGAACAGCGACAAGC |
| | | UGAUCGCCCGCAAGAAGGACUGGGACCCCAAGAAGUACGGUGGCUUCGACAGCCC |
| | | CACCGUGGCCUACAGCGUGCUGGUGGUGGCCAAGGUGGAGAAGGGGAAGAGCAAG |
| | | AAGCUGAAGAGCGUGAAGGAGCUGCUGGGCAUCACGAUCAUGGAGCGGAGCAGCU |
| | | UCGAGAAGAACCCCAUCGACUUCCUGGAAGCCAAGGGGUACAAGGAAGUCAAGAA |
| | | GGACCUGAUCAUCAAGCUUCCAAAGUACAGCCUGUUCGAGCUGGAGAAUGGGCGG |
| | | AAGCGGAUGCUGGCCAGCGCCGGUGAGCUGCAGAAGGGGAACGAGCUGGCACUUC |
| | | CCUCAAAGUACGUGAACUUCCUGUACCUGGCCAGCCACUACGAGAAGCUGAAGGG |
| | | GAGCCCAGAGGACAACGAGCAGAAGCAGCUGUUCGUGGAGCAGCACAAGCACUAC |
| | | CUGGACGAGAUCAUCGAGCAGAUCAGCGAGUUCAGCAAGCGGGUGAUCCUGGCCG |
| | | ACGCCAAUCUCGACAAGGUGCUCAGCGCCUACAACAAGCACCGAGACAAGCCCAU |
| | | CAGGGAGCAGGCCGAGAACAUCAUCCACCUGUUCACGCUGACGAAUCUCGGUGCC |
| | | CCCGCUGCCUUCAAGUACUUCGACACGACGAUCGACCGGAAGCGGUACACGUCGA |
| | | CUAAGGAAGUCCUGGACGCCACGCUGAUCCACCAGAGCAUCACGGGCCUGUACGA |
| | | GACGCGGAUCGACCUCAGCCAGCUGGGUGGCGACGGUGGUGGCAGCCCCAAGAAG |
| | | AAGCGGAAGGUGUAG |
| 664 | ORF encoding Sp. | AUGGACAAGAAGUACAGCAUCGGCCUCGACAUCGGCACCAACAGCGUCGGCUGGG |
| | Cas9 | CCGUCAUCACCGACGAGUACAAGGUCCCCAGCAAGAAGUUCAAGGUCCUCGGCAA |
| | | CACCGACCGCCACAGCAUCAAGAAGAACCUCAUCGGCGCCCUCCUCUUCGACAGC |
| | | GGCGAGACCGCCGAGGCCACCCGCCUCAAGCGCACCGCCCGCCGCCGCUACACCC |
| | | GCCGCAAGAACCGCAUCUGCUACCUCCAGGAGAUCUUCAGCAACGAGAUGGCCAA |
| | | GGUCGACGACAGCUUCUUCCACCGCCUCGAGGAGAGCUUCCUCGUCGAGGAGGAC |
| | | AAGAAGCACGAGCGCCACCCCAUCUUCGGCAACAUCGUCGACGAGGUCGCCUACC |
| | | ACGAGAAGUACCCCACCAUCUACCACCUCCGCAAGAAGCUCGUCGACAGCACCGA |
| | | CAAGGCCGACCUCCGCCUCAUCUACCUCGCCCUCGCCCACAUGAUCAAGUUCCGC |
| | | GGCCACUUCCUCAUCGAGGGCGACCUCAACCCCGACAACAGCGACGUCGACAAGC |
| | | UCUUCAUCCAGCUCGUCCAGACCUACAACCAGCUCUUCGAGGAGAACCCCAUCAA |
| | | CGCCAGCGGCGUCGACGCCAAGGCCAUCCUCAGCGCCCGCCUCAGCAAGAGCCGC |
| | | CGCCUCGAGAACCUCAUCGCCCAGCUCCCCGGCGAGAAGAAGAACGGCCUCUUCG |
| | | GCAACCUCAUCGCCCUCAGCCUCGGCCUCACCCCCAACUUCAAGAGCAACUUCGA |
| | | CCUCGCCGAGGACGCCAAGCUCCAGCUCAGCAAGGACACCUACGACGACGACCUC |
| | | GACAACCUCCUCGCCCAGAUCGGCGACCAGUACGCCGACCUCUUCCUCGCCGCCA |
| | | AGAACCUCAGCGACGCCAUCCUCCUCAGCGACAUCCUCCGCGUCAACACCGAGAU |
| | | CACCAAGGCCCCCCUCAGCGCCAGCAUGAUCAAGCGCUACGACGAGCACCACCAG |
| | | GACCUCACCCUCCUCAAGGCCCUCGUCCGCCAGCAGCUCCCCGAGAAGUACAAGG |
| | | AGAUCUUCUUCGACCAGAGCAAGAACGGCUACGCCGGCUACAUCGACGGCGGCGC |
| | | CAGCCAGGAGGAGUUCUACAAGUUCAUCAAGCCCAUCCUCGAGAAGAUGGACGGC |
| | | ACCGAGGAGCUCCUCGUCAAGCUCAACCGCGAGGACCUCCUCCGCAAGCAGCGCA |
| | | CCUUCGACAACGGCAGCAUCCCCCACCAGAUCCACCUCGGCGAGCUCCACGCCAU |
| | | CCUCCGCCGCCAGGAGGACUUCUACCCCUUCCUCAAGGACAACCGCGAGAAGAUC |
| | | GAGAAGAUCCUCACCUUCCGCAUCCCCUACUACGUCGGCCCCCUCGCCCGCGGCA |
| | | ACAGCCGCUUCGCCUGGAUGACCCGCAAGAGCGAGGAGACCAUCACCCCCUGGAA |
| | | CUUCGAGGAGGUCGUCGACAAGGGCGCCAGCGCCCAGAGCUUCAUCGAGCGCAUG |
| | | ACCAACUUCGACAAGAACCUCCCCAACGAGAAGGUCCUCCCCAAGCACAGCCUCC |
| | | UCUACGAGUACUUCACCGUCUACAACGAGCUCACCAAGGUCAAGUACGUCACCGA |
| | | GGGCAUGCGCAAGCCCGCCUUCCUCAGCGGCGAGCAGAAGAAGGCCAUCGUCGAC |
| | | CUCCUCUUCAAGACCAACCGCAAGGUCACCGUCAAGCAGCUCAAGGAGGACUACU |
| | | UCAAGAAGAUCGAGUGCUUCGACAGCGUCGAGAUCAGCGGCGUCGAGGACCGCUU |
| | | CAACGCCAGCCUCGGCACCUACCACGACCUCCUCAAGAUCAUCAAGGACAAGGAC |
| | | UUCCUCGACAACGAGGAGAACGAGGACAUCCUCGAGGACAUCGUCCUCACCCUCA |
| | | CCCUCUUCGAGGACCGCGAGAUGAUCGAGGAGCGCCUCAAGACCUACGCCCACCU |
| | | CUUCGACGACAAGGUCAUGAAGCAGCUCAAGCGCCGCCGCUACACCGGCUGGGGC |
| | | CGCCUCAGCCGCAAGCUCAUCAACGGCAUCCGCGACAAGCAGAGCGGCAAGACCA |
| | | UCCUCGACUUCCUCAAGAGCGACGGCUUCGCCAACCGCAACUUCAUGCAGCUCAU |
| | | CCACGACGACAGCCUCACCUUCAAGGAGGACAUCCAGAAGGCCCAGGUCAGCGGC |
| | | CAGGGCGACAGCCUCCACGAGCACAUCGCCAACCUCGCCGGCAGCCCCGCCAUCA |
| | | AGAAGGGCAUCCUCCAGACCGUCAAGGUCGUCGACGAGCUCGUCAAGGUCAUGGG |
| | | CCGCCACAAGCCCGAGAACAUCGUCAUCGAGAUGGCCCGCGAGAACCAGACCACC |
| | | CAGAAGGGCCAGAAGAACAGCCGCGAGCGCAUGAAGCGCAUCGAGGAGGGCAUCA |
| | | AGGAGCUCGGCAGCCAGAUCCUCAAGGAGCACCCCGUCGAGAACACCCAGCUCCA |
| | | GAACGAGAAGCUCUACCUCUACUACCUCCAGAACGGCCGCGACAUGUACGUCGAC |
| | | CAGGAGCUCGACAUCAACCGCCUCAGCGACUACGACGUCGACCACAUCGUCCCCC |
| | | AGAGCUUCCUCAAGGACGACAGCAUCGACAACAAGGUCCUCACCCGCAGCGACAA |
| | | GAACCGCGGCAAGAGCGACAACGUCCCCAGCGAGGAGGUCGUCAAGAAGAUGAAG |
| | | AACUACUGGCGCCAGCUCCUCAACGCCAAGCUCAUCACCCAGCGCAAGUUCGACA |
| | | ACCUCACCAAGGCCGAGCGCGGCGGCCUCAGCGAGCUCGACAAGGCCGGCUUCAU |
| | | CAAGCGCCAGCUCGUCGAGACCCGCCAGAUCACCAAGCACGUCGCCCAGAUCCUC |
| | | GACAGCCGCAUGAACACCAAGUACGACGAGAACGACAAGCUCAUCCGCGAGGUCA |
| | | AGGUCAUCACCCUCAAGAGCAAGCUCGUCAGCGACUUCCGCAAGGACUUCCAGUU |
| | | CUACAAGGUCCGCGAGAUCAACAACUACCACCACGCCCACGACGCCUACCUCAAC |
| | | GCCGUCGUCGGCACCGCCCUCAUCAAGAAGUACCCCAAGCUCGAGAGCGAGUUCG |
| | | UCUACGGCGACUACAAGGUCUACGACGUCCGCAAGAUGAUCGCCAAGAGCGAGCA |
| | | GGAGAUCGGCAAGGCCACCGCCAAGUACUUCUUCUACAGCAACAUCAUGAACUUC |
| | | UUCAAGACCGAGAUCACCCUCGCCAACGGCGAGAUCCGCAAGCGCCCCCUCAUCG |
| | | AGACCAACGGCGAGACCGGCGAGAUCGUCUGGGACAAGGGCCGCGACUUCGCCAC |
| | | CGUCCGCAAGGUCCUCAGCAUGCCCCAGGUCAACAUCGUCAAGAAGACCGAGGUC |
| | | CAGACCGGCGGCUUCAGCAAGGAGAGCAUCCUCCCCAAGCGCAACAGCGACAAGC |
| | | UCAUCGCCCGCAAGAAGGACUGGGACCCCAAGAAGUACGGCGGCUUCGACAGCCC |
| | | CACCGUCGCCUACAGCGUCCUCGUCGUCGCCAAGGUCGAGAAGGGCAAGAGCAAG |
| | | AAGCUCAAGAGCGUCAAGGAGCUCCUCGGCAUCACCAUCAUGGAGCGCAGCAGCU |
| | | UCGAGAAGAACCCCAUCGACUUCCUCGAGGCCAAGGGCUACAAGGAGGUCAAGAA |
| | | GGACCUCAUCAUCAAGCUCCCCAAGUACAGCCUCUUCGAGCUCGAGAACGGCCGC |
| | | AAGCGCAUGCUCGCCAGCGCCGGCGAGCUCCAGAAGGGCAACGAGCUCGCCCUCC |
| | | CCAGCAAGUACGUCAACUUCCUCUACCUCGCCAGCCACUACGAGAAGCUCAAGGG |
| | | CAGCCCCGAGGACAACGAGCAGAAGCAGCUCUUCGUCGAGCAGCACAAGCACUAC |
| | | CUCGACGAGAUCAUCGAGCAGAUCAGCGAGUUCAGCAAGCGCGUCAUCCUCGCCG |
| | | ACGCCAACCUCGACAAGGUCCUCAGCGCCUACAACAAGCACCGCGACAAGCCCAU |
| | | CCGCGAGCAGGCCGAGAACAUCAUCCACCUCUUCACCCUCACCAACCUCGGCGCC |
| | | CCCGCCGCCUUCAAGUACUUCGACACCACCAUCGACCGCAAGCGCUACACCAGCA |
| | | CCAAGGAGGUCCUCGACGCCACCCUCAUCCACCAGAGCAUCACCGGCCUCUACGA |
| | | GACCCGCAUCGACCUCAGCCAGCUCGGCGGCGACGGCGGCGGCAGCCCCAAGAAG |
| | | AAGCGCAAGGUCUAG |
| 665 | ORF encoding Sp. | ATGGACAAGAAGTACAGCATCGGCCTGGACATCGGCACCAACAGCGTGGGCTGGG |
| | Cas9 | CCGTGATCACCGACGAGTACAAGGTGCCCAGCAAGAAGTTCAAGGTGCTGGGCAA |
| | | CACCGACCGGCACAGCATCAAGAAGAACCTGATCGGCGCCCTGCTGTTCGACAGC |
| | | GGCGAGACCGCCGAGGCCACCCGGCTGAAGCGGACCGCCCGGCGGCGGTACACCC |
| | | GGCGGAAGAACCGGATCTGCTACCTGCAGGAGATCTTCAGCAACGAGATGGCCAA |
| | | GGTGGACGACAGCTTCTTCCACCGGCTGGAGGAGAGCTTCCTGGTGGAGGAGGAC |
| | | AAGAAGCACGAGCGGCACCCCATCTTCGGCAACATCGTGGACGAGGTGGCCTACC |
| | | ACGAGAAGTACCCCACCATCTACCACCTGCGGAAGAAGCTGGTGGACAGCACCGA |
| | | CAAGGCCGACCTGCGGCTGATCTACCTGGCCCTGGCCCACATGATCAAGTTCCGG |
| | | GGCCACTTCCTGATCGAGGGCGACCTGAACCCCGACAACAGCGACGTGGACAAGC |
| | | TGTTCATCCAGCTGGTGCAGACCTACAACCAGCTGTTCGAGGAGAACCCCATCAA |
| | | CGCCAGCGGCGTGGACGCCAAGGCCATCCTGAGCGCCCGGCTGAGCAAGAGCCGG |
| | | CGGCTGGAGAACCTGATCGCCCAGCTGCCCGGCGAGAAGAAGAACGGCCTGTTCG |
| | | GCAACCTGATCGCCCTGAGCCTGGGCCTGACCCCCAACTTCAAGAGCAACTTCGA |
| | | CCTGGCCGAGGACGCCAAGCTGCAGCTGAGCAAGGACACCTACGACGACGACCTG |
| | | GACAACCTGCTGGCCCAGATCGGCGACCAGTACGCCGACCTGTTCCTGGCCGCCA |
| | | AGAACCTGAGCGACGCCATCCTGCTGAGCGACATCCTGCGGGTGAACACCGAGAT |
| | | CACCAAGGCCCCCCTGAGCGCCAGCATGATCAAGCGGTACGACGAGCACCACCAG |
| | | GACCTGACCCTGCTGAAGGCCCTGGTGCGGCAGCAGCTGCCCGAGAAGTACAAGG |
| | | AGATCTTCTTCGACCAGAGCAAGAACGGCTACGCCGGCTACATCGACGGCGGCGC |
| | | CAGCCAGGAGGAGTTCTACAAGTTCATCAAGCCCATCCTGGAGAAGATGGACGGC |
| | | ACCGAGGAGCTGCTGGTGAAGCTGAACCGGGAGGACCTGCTGCGGAAGCAGCGGA |
| | | CCTTCGACAACGGCAGCATCCCCCACCAGATCCACCTGGGCGAGCTGCACGCCAT |
| | | CCTGCGGCGGCAGGAGGACTTCTACCCCTTCCTGAAGGACAACCGGGAGAAGATC |
| | | GAGAAGATCCTGACCTTCCGGATCCCCTACTACGTGGGCCCCCTGGCCCGGGGCA |
| | | ACAGCCGGTTCGCCTGGATGACCCGGAAGAGCGAGGAGACCATCACCCCCTGGAA |
| | | CTTCGAGGAGGTGGTGGACAAGGGCGCCAGCGCCCAGAGCTTCATCGAGCGGATG |
| | | ACCAACTTCGACAAGAACCTGCCCAACGAGAAGGTGCTGCCCAAGCACAGCCTGC |
| | | TGTACGAGTACTTCACCGTGTACAACGAGCTGACCAAGGTGAAGTACGTGACCGA |
| | | GGGCATGCGGAAGCCCGCCTTCCTGAGCGGCGAGCAGAAGAAGGCCATCGTGGAC |
| | | CTGCTGTTCAAGACCAACCGGAAGGTGACCGTGAAGCAGCTGAAGGAGGACTACT |
| | | TCAAGAAGATCGAGTGCTTCGACAGCGTGGAGATCAGCGGCGTGGAGGACCGGTT |
| | | CAACGCCAGCCTGGGCACCTACCACGACCTGCTGAAGATCATCAAGGACAAGGAC |
| | | TTCCTGGACAACGAGGAGAACGAGGACATCCTGGAGGACATCGTGCTGACCCTGA |
| | | CCCTGTTCGAGGACCGGGAGATGATCGAGGAGCGGCTGAAGACCTACGCCCACCT |
| | | GTTCGACGACAAGGTGATGAAGCAGCTGAAGCGGCGGCGGTACACCGGCTGGGGC |
| | | CGGCTGAGCCGGAAGCTGATCAACGGCATCCGGGACAAGCAGAGCGGCAAGACCA |
| | | TCCTGGACTTCCTGAAGAGCGACGGCTTCGCCAACCGGAACTTCATGCAGCTGAT |
| | | CCACGACGACAGCCTGACCTTCAAGGAGGACATCCAGAAGGCCCAGGTGAGCGGC |
| | | CAGGGCGACAGCCTGCACGAGCACATCGCCAACCTGGCCGGCAGCCCCGCCATCA |
| | | AGAAGGGCATCCTGCAGACCGTGAAGGTGGTGGACGAGCTGGTGAAGGTGATGGG |
| | | CCGGCACAAGCCCGAGAACATCGTGATCGAGATGGCCCGGGAGAACCAGACCACC |
| | | CAGAAGGGCCAGAAGAACAGCCGGGAGCGGATGAAGCGGATCGAGGAGGGCATCA |
| | | AGGAGCTGGGCAGCCAGATCCTGAAGGAGCACCCCGTGGAGAACACCCAGCTGCA |
| | | GAACGAGAAGCTGTACCTGTACTACCTGCAGAACGGCCGGGACATGTACGTGGAC |
| | | CAGGAGCTGGACATCAACCGGCTGAGCGACTACGACGTGGACCACATCGTGCCCC |
| | | AGAGCTTCCTGAAGGACGACAGCATCGACAACAAGGTGCTGACCCGGAGCGACAA |
| | | GAACCGGGGCAAGAGCGACAACGTGCCCAGCGAGGAGGTGGTGAAGAAGATGAAG |
| | | AACTACTGGCGGCAGCTGCTGAACGCCAAGCTGATCACCCAGCGGAAGTTCGACA |
| | | ACCTGACCAAGGCCGAGCGGGGCGGCCTGAGCGAGCTGGACAAGGCCGGCTTCAT |
| | | CAAGCGGCAGCTGGTGGAGACCCGGCAGATCACCAAGCACGTGGCCCAGATCCTG |
| | | GACAGCCGGATGAACACCAAGTACGACGAGAACGACAAGCTGATCCGGGAGGTGA |
| | | AGGTGATCACCCTGAAGAGCAAGCTGGTGAGCGACTTCCGGAAGGACTTCCAGTT |
| | | CTACAAGGTGCGGGAGATCAACAACTACCACCACGCCCACGACGCCTACCTGAAC |
| | | GCCGTGGTGGGCACCGCCCTGATCAAGAAGTACCCCAAGCTGGAGAGCGAGTTCG |
| | | TGTACGGCGACTACAAGGTGTACGACGTGCGGAAGATGATCGCCAAGAGCGAGCA |
| | | GGAGATCGGCAAGGCCACCGCCAAGTACTTCTTCTACAGCAACATCATGAACTTC |
| | | TTCAAGACCGAGATCACCCTGGCCAACGGCGAGATCCGGAAGCGGCCCCTGATCG |
| | | AGACCAACGGCGAGACCGGCGAGATCGTGTGGGACAAGGGCCGGGACTTCGCCAC |
| | | CGTGCGGAAGGTGCTGAGCATGCCCCAGGTGAACATCGTGAAGAAGACCGAGGTG |
| | | CAGACCGGCGGCTTCAGCAAGGAGAGCATCCTGCCCAAGCGGAACAGCGACAAGC |
| | | TGATCGCCCGGAAGAAGGACTGGGACCCCAAGAAGTACGGCGGCTTCGACAGCCC |
| | | CACCGTGGCCTACAGCGTGCTGGTGGTGGCCAAGGTGGAGAAGGGCAAGAGCAAG |
| | | AAGCTGAAGAGCGTGAAGGAGCTGCTGGGCATCACCATCATGGAGCGGAGCAGCT |
| | | TCGAGAAGAACCCCATCGACTTCCTGGAGGCCAAGGGCTACAAGGAGGTGAAGAA |
| | | GGACCTGATCATCAAGCTGCCCAAGTACAGCCTGTTCGAGCTGGAGAACGGCCGG |
| | | AAGCGGATGCTGGCCAGCGCCGGCGAGCTGCAGAAGGGCAACGAGCTGGCCCTGC |
| | | CCAGCAAGTACGTGAACTTCCTGTACCTGGCCAGCCACTACGAGAAGCTGAAGGG |
| | | CAGCCCCGAGGACAACGAGCAGAAGCAGCTGTTCGTGGAGCAGCACAAGCACTAC |
| | | CTGGACGAGATCATCGAGCAGATCAGCGAGTTCAGCAAGCGGGTGATCCTGGCCG |
| | | ACGCCAACCTGGACAAGGTGCTGAGCGCCTACAACAAGCACCGGGACAAGCCCAT |
| | | CCGGGAGCAGGCCGAGAACATCATCCACCTGTTCACCCTGACCAACCTGGGCGCC |
| | | CCCGCCGCCTTCAAGTACTTCGACACCACCATCGACCGGAAGCGGTACACCAGCA |
| | | CCAAGGAGGTGCTGGACGCCACCCTGATCCACCAGAGCATCACCGGCCTGTACGA |
| | | GACCCGGATCGACCTGAGCCAGCTGGGCGGCGACGGCGGCGGCAGCCCCAAGAAG |
| | | AAGCGGAAGGTGTGA |
| 666 | amino acid | MDKKYSIGLDIGTNSVGWAVITDEYKVPSKKFKVLGNTDRHSIKKNLIGALLFDS |
| | sequence for Sp. | GETAEATRLKRTARRRYTRRKNRICYLQEIFSNEMAKVDDSFFHRLEESFLVEED |
| | Cas9 | KKHERHPIFGNIVDEVAYHEKYPTIYHLRKKLVDSTDKADLRLIYLALAHMIKER |
| | | GHFLIEGDLNPDNSDVDKLFIQLVQTYNQLFEENPINASGVDAKAILSARLSKSR |
| | | RLENLIAQLPGEKKNGLFGNLIALSLGLTPNEKSNFDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTEDNGSIPHQIHLGELHAILRRQEDFYPFLKDNREKI |
| | | EKILTFRIPYYVGPLARGNSRFAWMTRKSEETITPWNFEEVVDKGASAQSFIERM |
| | | TNEDKNLPNEKVLPKHSLLYEYFTVYNELTKVKYVTEGMRKPAFLSGEQKKAIVD |
| | | LLEKTNRKVTVKQLKEDYFKKIECFDSVEISGVEDRENASLGTYHDLLKIIKDKD |
| | | ELDNEENEDILEDIVLTLTLFEDREMIEERLKTYAHLEDDKVMKQLKRRRYTGWG |
| | | RLSRKLINGIRDKQSGKTILDELKSDGFANRNFMQLIHDDSLTFKEDIQKAQVSG |
| | | QGDSLHEHIANLAGSPAIKKGILQTVKVVDELVKVMGRHKPENIVIEMARENQTT |
| | | QKGQKNSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVVKKMK |
| | | NYWRQLLNAKLITQRKEDNLTKAERGGLSELDKAGFIKRQLVETRQITKHVAQIL |
| | | DSRMNTKYDENDKLIREVKVITLKSKLVSDERKDFQFYKVREINNYHHAHDAYLN |
| | | AVVGTALIKKYPKLESEFVYGDYKVYDVRKMIAKSEQEIGKATAKYFFYSNIMNF |
| | | FKTEITLANGEIRKRPLIETNGETGEIVWDKGRDFATVRKVLSMPQVNIVKKTEV |
| | | QTGGESKESILPKRNSDKLIARKKDWDPKKYGGEDSPTVAYSVLVVAKVEKGKSK |
| | | KLKSVKELLGITIMERSSFEKNPIDELEAKGYKEVKKDLIIKLPKYSLFELENGR |
| | | KRMLASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLTNLGA |
| | | PAAFKYEDTTIDRKRYTSTKEVLDATLIHQSITGLYETRIDLSQLGGDGGGSPKK |
| | | KRKV |
| 667 | Open reading | AUGGACAAGAAGUACUCCAUCGGCCUGGACAUCGGCACCAACUCCGUGGGCUGGG |
| | frame for Cas9 | CCGUGAUCACCGACGAGUACAAGGUGCCCUCCAAGAAGUUCAAGGUGCUGGGCAA |
| | with HiBiT tag | CACCGACCGGCACUCCAUCAAGAAGAACCUGAUCGGCGCCCUGCUGUUCGACUCC |
| | | GGCGAGACCGCCGAGGCCACCCGGCUGAAGCGGACCGCCCGGCGGCGGUACACCC |
| | | GGCGGAAGAACCGGAUCUGCUACCUGCAGGAGAUCUUCUCCAACGAGAUGGCCAA |
| | | GGUGGACGACUCCUUCUUCCACCGGCUGGAGGAGUCCUUCCUGGUGGAGGAGGAC |
| | | AAGAAGCACGAGCGGCACCCCAUCUUCGGCAACAUCGUGGACGAGGUGGCCUACC |
| | | ACGAGAAGUACCCCACCAUCUACCACCUGCGGAAGAAGCUGGUGGACUCCACCGA |
| | | CAAGGCCGACCUGCGGCUGAUCUACCUGGCCCUGGCCCACAUGAUCAAGUUCCGG |
| | | GGCCACUUCCUGAUCGAGGGCGACCUGAACCCCGACAACUCCGACGUGGACAAGC |
| | | UGUUCAUCCAGCUGGUGCAGACCUACAACCAGCUGUUCGAGGAGAACCCCAUCAA |
| | | CGCCUCCGGCGUGGACGCCAAGGCCAUCCUGUCCGCCCGGCUGUCCAAGUCCCGG |
| | | CGGCUGGAGAACCUGAUCGCCCAGCUGCCCGGCGAGAAGAAGAACGGCCUGUUCG |
| | | GCAACCUGAUCGCCCUGUCCCUGGGCCUGACCCCCAACUUCAAGUCCAACUUCGA |
| | | CCUGGCCGAGGACGCCAAGCUGCAGCUGUCCAAGGACACCUACGACGACGACCUG |
| | | GACAACCUGCUGGCCCAGAUCGGCGACCAGUACGCCGACCUGUUCCUGGCCGCCA |
| | | AGAACCUGUCCGACGCCAUCCUGCUGUCCGACAUCCUGCGGGUGAACACCGAGAU |
| | | CACCAAGGCCCCCCUGUCCGCCUCCAUGAUCAAGCGGUACGACGAGCACCACCAG |
| | | GACCUGACCCUGCUGAAGGCCCUGGUGCGGCAGCAGCUGCCCGAGAAGUACAAGG |
| | | AGAUCUUCUUCGACCAGUCCAAGAACGGCUACGCCGGCUACAUCGACGGCGGCGC |
| | | CUCCCAGGAGGAGUUCUACAAGUUCAUCAAGCCCAUCCUGGAGAAGAUGGACGGC |
| | | ACCGAGGAGCUGCUGGUGAAGCUGAACCGGGAGGACCUGCUGCGGAAGCAGCGGA |
| | | CCUUCGACAACGGCUCCAUCCCCCACCAGAUCCACCUGGGCGAGCUGCACGCCAU |
| | | CCUGCGGCGGCAGGAGGACUUCUACCCCUUCCUGAAGGACAACCGGGAGAAGAUC |
| | | GAGAAGAUCCUGACCUUCCGGAUCCCCUACUACGUGGGCCCCCUGGCCCGGGGCA |
| | | ACUCCCGGUUCGCCUGGAUGACCCGGAAGUCCGAGGAGACCAUCACCCCCUGGAA |
| | | CUUCGAGGAGGUGGUGGACAAGGGCGCCUCCGCCCAGUCCUUCAUCGAGCGGAUG |
| | | ACCAACUUCGACAAGAACCUGCCCAACGAGAAGGUGCUGCCCAAGCACUCCCUGC |
| | | UGUACGAGUACUUCACCGUGUACAACGAGCUGACCAAGGUGAAGUACGUGACCGA |
| | | GGGCAUGCGGAAGCCCGCCUUCCUGUCCGGCGAGCAGAAGAAGGCCAUCGUGGAC |
| | | CUGCUGUUCAAGACCAACCGGAAGGUGACCGUGAAGCAGCUGAAGGAGGACUACU |
| | | UCAAGAAGAUCGAGUGCUUCGACUCCGUGGAGAUCUCCGGCGUGGAGGACCGGUU |
| | | CAACGCCUCCCUGGGCACCUACCACGACCUGCUGAAGAUCAUCAAGGACAAGGAC |
| | | UUCCUGGACAACGAGGAGAACGAGGACAUCCUGGAGGACAUCGUGCUGACCCUGA |
| | | CCCUGUUCGAGGACCGGGAGAUGAUCGAGGAGCGGCUGAAGACCUACGCCCACCU |
| | | GUUCGACGACAAGGUGAUGAAGCAGCUGAAGCGGCGGCGGUACACCGGCUGGGGC |
| | | CGGCUGUCCCGGAAGCUGAUCAACGGCAUCCGGGACAAGCAGUCCGGCAAGACCA |
| | | UCCUGGACUUCCUGAAGUCCGACGGCUUCGCCAACCGGAACUUCAUGCAGCUGAU |
| | | CCACGACGACUCCCUGACCUUCAAGGAGGACAUCCAGAAGGCCCAGGUGUCCGGC |
| | | CAGGGCGACUCCCUGCACGAGCACAUCGCCAACCUGGCCGGCUCCCCCGCCAUCA |
| | | AGAAGGGCAUCCUGCAGACCGUGAAGGUGGUGGACGAGCUGGUGAAGGUGAUGGG |
| | | CCGGCACAAGCCCGAGAACAUCGUGAUCGAGAUGGCCCGGGAGAACCAGACCACC |
| | | CAGAAGGGCCAGAAGAACUCCCGGGAGCGGAUGAAGCGGAUCGAGGAGGGCAUCA |
| | | AGGAGCUGGGCUCCCAGAUCCUGAAGGAGCACCCCGUGGAGAACACCCAGCUGCA |
| | | GAACGAGAAGCUGUACCUGUACUACCUGCAGAACGGCCGGGACAUGUACGUGGAC |
| | | CAGGAGCUGGACAUCAACCGGCUGUCCGACUACGACGUGGACCACAUCGUGCCCC |
| | | AGUCCUUCCUGAAGGACGACUCCAUCGACAACAAGGUGCUGACCCGGUCCGACAA |
| | | GAACCGGGGCAAGUCCGACAACGUGCCCUCCGAGGAGGUGGUGAAGAAGAUGAAG |
| | | AACUACUGGCGGCAGCUGCUGAACGCCAAGCUGAUCACCCAGCGGAAGUUCGACA |
| | | ACCUGACCAAGGCCGAGCGGGGCGGCCUGUCCGAGCUGGACAAGGCCGGCUUCAU |
| | | CAAGCGGCAGCUGGUGGAGACCCGGCAGAUCACCAAGCACGUGGCCCAGAUCCUG |
| | | GACUCCCGGAUGAACACCAAGUACGACGAGAACGACAAGCUGAUCCGGGAGGUGA |
| | | AGGUGAUCACCCUGAAGUCCAAGCUGGUGUCCGACUUCCGGAAGGACUUCCAGUU |
| | | CUACAAGGUGCGGGAGAUCAACAACUACCACCACGCCCACGACGCCUACCUGAAC |
| | | GCCGUGGUGGGCACCGCCCUGAUCAAGAAGUACCCCAAGCUGGAGUCCGAGUUCG |
| | | UGUACGGCGACUACAAGGUGUACGACGUGCGGAAGAUGAUCGCCAAGUCCGAGCA |
| | | GGAGAUCGGCAAGGCCACCGCCAAGUACUUCUUCUACUCCAACAUCAUGAACUUC |
| | | UUCAAGACCGAGAUCACCCUGGCCAACGGCGAGAUCCGGAAGCGGCCCCUGAUCG |
| | | AGACCAACGGCGAGACCGGCGAGAUCGUGUGGGACAAGGGCCGGGACUUCGCCAC |
| | | CGUGCGGAAGGUGCUGUCCAUGCCCCAGGUGAACAUCGUGAAGAAGACCGAGGUG |
| | | CAGACCGGCGGCUUCUCCAAGGAGUCCAUCCUGCCCAAGCGGAACUCCGACAAGC |
| | | UGAUCGCCCGGAAGAAGGACUGGGACCCCAAGAAGUACGGCGGCUUCGACUCCCC |
| | | CACCGUGGCCUACUCCGUGCUGGUGGUGGCCAAGGUGGAGAAGGGCAAGUCCAAG |
| | | AAGCUGAAGUCCGUGAAGGAGCUGCUGGGCAUCACCAUCAUGGAGCGGUCCUCCU |
| | | UCGAGAAGAACCCCAUCGACUUCCUGGAGGCCAAGGGCUACAAGGAGGUGAAGAA |
| | | GGACCUGAUCAUCAAGCUGCCCAAGUACUCCCUGUUCGAGCUGGAGAACGGCCGG |
| | | AAGCGGAUGCUGGCCUCCGCCGGCGAGCUGCAGAAGGGCAACGAGCUGGCCCUGC |
| | | CCUCCAAGUACGUGAACUUCCUGUACCUGGCCUCCCACUACGAGAAGCUGAAGGG |
| | | CUCCCCCGAGGACAACGAGCAGAAGCAGCUGUUCGUGGAGCAGCACAAGCACUAC |
| | | CUGGACGAGAUCAUCGAGCAGAUCUCCGAGUUCUCCAAGCGGGUGAUCCUGGCCG |
| | | ACGCCAACCUGGACAAGGUGCUGUCCGCCUACAACAAGCACCGGGACAAGCCCAU |
| | | CCGGGAGCAGGCCGAGAACAUCAUCCACCUGUUCACCCUGACCAACCUGGGCGCC |
| | | CCCGCCGCCUUCAAGUACUUCGACACCACCAUCGACCGGAAGCGGUACACCUCCA |
| | | CCAAGGAGGUGCUGGACGCCACCCUGAUCCACCAGUCCAUCACCGGCCUGUACGA |
| | | GACCCGGAUCGACCUGUCCCAGCUGGGCGGCGACGGCGGCGGCUCCCCCAAGAAG |
| | | AAGCGGAAGGUGUCCGAGUCCGCCACCCCCGAGUCCGUGUCCGGCUGGCGGCUGU |
| | | UCAAGAAGAUCUCCUGA |
| 668 | Amino acid | MDKKYSIGLDIGTNSVGWAVITDEYKVPSKKFKVLGNTDRHSIKKNLIGALLEDS |
| | sequence for | GETAEATRLKRTARRRYTRRKNRICYLQEIFSNEMAKVDDSFFHRLEESFLVEED |
| | Cas9 with HiBiT | KKHERHPIFGNIVDEVAYHEKYPTIYHLRKKLVDSTDKADLRLIYLALAHMIKER |
| | tag | GHFLIEGDLNPDNSDVDKLFIQLVQTYNQLFEENPINASGVDAKAILSARLSKSR |
| | | RLENLIAQLPGEKKNGLFGNLIALSLGLTPNEKSNFDLAEDAKLQLSKDTYDDDL |
| | | DNLLAQIGDQYADLELAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTEDNGSIPHQIHLGELHAILRRQEDFYPFLKDNREKI |
| | | EKILTFRIPYYVGPLARGNSRFAWMTRKSEETITPWNFEEVVDKGASAQSFIERM |
| | | TNEDKNLPNEKVLPKHSLLYEYFTVYNELTKVKYVTEGMRKPAFLSGEQKKAIVD |
| | | LLFKTNRKVTVKQLKEDYFKKIECFDSVEISGVEDRENASLGTYHDLLKIIKDKD |
| | | ELDNEENEDILEDIVLTLTLFEDREMIEERLKTYAHLEDDKVMKQLKRRRYTGWG |
| | | RLSRKLINGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEMARENQTT |
| | | QKGQKNSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQIL |
| | | DSRMNTKYDENDKLIREVKVITLKSKLVSDERKDFQFYKVREINNYHHAHDAYLN |
| | | AVVGTALIKKYPKLESEFVYGDYKVYDVRKMIAKSEQEIGKATAKYFFYSNIMNF |
| | | FKTEITLANGEIRKRPLIETNGETGEIVWDKGRDFATVRKVLSMPQVNIVKKTEV |
| | | QTGGFSKESILPKRNSDKLIARKKDWDPKKYGGEDSPTVAYSVLVVAKVEKGKSK |
| | | KLKSVKELLGITIMERSSFEKNPIDELEAKGYKEVKKDLIIKLPKYSLFELENGR |
| | | KRMLASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLTNLGA |
| | | PAAFKYFDTTIDRKRYTSTKEVLDATLIHQSITGLYETRIDLSQLGGDGGGSPKK |
| | | KRKVSESATPESVSGWRLFKKIS |
| 669 | SV40 NLS | PKKKRKV |
| 670 | Alternate SV40 | PKKKRRV |
| | NLS | |
| 671 | Exemplary NLS 1 | LAAKRSRTT |
| 672 | Exemplary NLS 2 | QAAKRSRTT |
| 673 | Exemplary NLS 3 | PAPAKRERTT |
| 674 | Exemplary NLS 4 | QAAKRPRTT |
| 675 | Exemplary NLS 5 | RAAKRPRTT |
| 676 | Exemplary NLS 6 | AAAKRSWSMAA |
| 677 | Exemplary NLS 7 | AAAKRVWSMAF |
| 678 | Exemplary NLS 8 | AAAKRSWSMAF |
| 679 | Exemplary NLS 9 | AAAKRKYFAA |
| 680 | Exemplary NLS | RAAKRKAFAA |
| | 10 | |
| 681 | Exemplary NLS | RAAKRKYFAV |
| | 11 | |
| 682 | Nucleoplasmin | KRPAATKKAGQAKKKK |
| | NLS | |
| 683 | Alternative | PKKKRKVE |
| | SV40 NLS | |
| 684 | Alternative | KKKRKVE |
| | SV40 NLS | |
| 685 | bipartite NLS | KRTADGSEFESPKKKRKVE |
| 686 | c-myc like NLS | PAAKKKKLD |
| 687-700 | Not used | |
| 701 | Exemplary | GGGAAGCUCAGAAUAAACGCUCAACUUUGGCCGGAUCUGCCACCAUGGACGGCUC |
| | sequence | CGGCGGCGGCUCCCCCAAGAAGAAGCGGAAGGUGGAGGACAAGCGGCCCGCCGCC |
| | encoding | ACCAAGAAGGCCGGCCAGGCCAAGAAGAAGAAGGGCGGCUCCGGCGGCGGCGCCG |
| | Nme2Cas 9 | CCUUCAAGCCCAACCCCAUCAACUACAUCCUGGGCCUGGACAUCGGCAUCGCCUC |
| | (mRNA | CGUGGGCUGGGCCAUGGUGGAGAUCGACGAGGAGGAGAACCCCAUCCGGCUGAUC |
| | AA) | GACCUGGGCGUGCGGGUGUUCGAGCGGGCCGAGGUGCCCAAGACCGGCGACUCCC |
| | | UGGCCAUGGCCCGGCGGCUGGCCCGGUCCGUGCGGCGGCUGACCCGGCGGCGGGC |
| | | CCACCGGCUGCUGCGGGCCCGGCGGCUGCUGAAGCGGGAGGGCGUGCUGCAGGCC |
| | | GCCGACUUCGACGAGAACGGCCUGAUCAAGUCCCUGCCCAACACCCCCUGGCAGC |
| | | UGCGGGCCGCCGCCCUGGACCGGAAGCUGACCCCCCUGGAGUGGUCCGCCGUGCU |
| | | GCUGCACCUGAUCAAGCACCGGGGCUACCUGUCCCAGCGGAAGAACGAGGGCGAG |
| | | ACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGA |
| | | GAAGGAGUCCGGCCACAUCCGGAACCAGCGGGGCGACUACUCCCACACCUUCUCC |
| | | CGGAAGGACCUGCAGGCCGAGCUGAUCCUGCUGUUCGAGAAGCAGAAGGAGUUCG |
| | | GCAACCCCCACGUGUCCGGCGGCCUGAAGGAGGGCAUCGAGACCCUGCUGAUGAC |
| | | CCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCA |
| | | UCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAG |
| | | CUGACCUACGCCCAGGCCCGGAAGCUGCUGGGCCUGGAGGACACCGCCUUCUUCA |
| | | AGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAG |
| | | UCCCCCCUGAACCUGUCCUCCGAGCUGCAGGACGAGAUCGGCACCGCCUUCUCCC |
| | | UGUUCAAGACCGACGAGGACAUCACCGGCCGGCUGAAGGACCGGGUGCAGCCCGA |
| | | GAUCCUGGAGGCCCUGCUGAAGCACAUCUCCUUCGACAAGUUCGUGCAGAUCUCC |
| | | CUGAAGGCCCUGCGGCGGAUCGUGCCCCUGAUGGAGCAGGGCAAGCGGUACGACG |
| | | AGGCCUGCGCCGAGAUCUACGGCGACCACUACGGCAAGAAGAACACCGAGGAGAA |
| | | GAUCUACCUGCCCCCCAUCCCCGCCGACGAGAUCCGGAACCCCGUGGUGCUGCGG |
| | | GCCCUGUCCCAGGCCCGGAAGGUGAUCAACGGCGUGGUGCGGCGGUACGGCUCCC |
| | | CCGCCCGGAUCCACAUCGAGACCGCCCGGGAGGUGGGCAAGUCCUUCAAGGACCG |
| | | GAAGGAGAUCGAGAAGCGGCAGGAGGAGAACCGGAAGGACCGGGAGAAGGCCGCC |
| | | GCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCC |
| | | CUGCCCUUCUCCCGGACCUGGGACGACUCCUUCAACAACAAGGUGCUGGUGCUGG |
| | | GCUCCGAGAACCAGAACAAGGGCAACCAGACCCCCUACGAGUACUUCAACGGCAA |
| | | GGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCA |
| | | AGGAGUGCAACCUGAACGACACCCGGUACGUGAACCGGUUCCUGUGCCAGUUCGU |
| | | GGCCGACCACAUCCUGCUGACCGGCAAGGGCAAGCGGCGGGUGUUCGCCUCCAAC |
| | | GGCCAGAUCACCAACCUGCUGCGGGGCUUCUGGGGCCUGCGGAAGGUGCGGGCCG |
| | | AGAACGACCGGCACCACGCCCUGGACGCCGUGGUGGUGGCCUGCUCCACCGUGGC |
| | | CAUGCAGCAGAAGAUCACCCGGUUCGUGCGGUACAAGGAGAUGAACGCCUUCGAC |
| | | GGCAAGACCAUCGACAAGGAGACCGGCAAGGUGCUGCACCAGAAGACCCACUUCC |
| | | CCCAGCCCUGGGAGUUCUUCGCCCAGGAGGUGAUGAUCCGGGUGUUCGGCAAGCC |
| | | CGACGGCAAGCCCGAGUUCGAGGAGGCCGACACCCCCGAGAAGCUGCGGACCCUG |
| | | CUGGCCGAGAAGCUGUCCUCCCGGCCCGAGGCCGUGCACGAGUACGUGACCCCCC |
| | | UGUUCGUGUCCCGGGCCCCCAACCGGAAGAUGUCCGGCGCCCACAAGGACACCCU |
| | | GCGGUCCGCCAAGCGGUUCGUGAAGCACAACGAGAAGAUCUCCGUGAAGCGGGUG |
| | | UGGCUGACCGAGAUCAAGCUGGCCGACCUGGAGAACAUGGUGAACUACAAGAACG |
| | | GCCGGGAGAUCGAGCUGUACGAGGCCCUGAAGGCCCGGCUGGAGGCCUACGGCGG |
| | | CAACGCCAAGCAGGCCUUCGACCCCAAGGACAACCCCUUCUACAAGAAGGGCGGC |
| | | CAGCUGGUGAAGGCCGUGCGGGUGGAGAAGACCCAGGAGUCCGGCGUGCUGCUGA |
| | | ACAAGAAGAACGCCUACACCAUCGCCGACAACGGCGACAUGGUGCGGGUGGACGU |
| | | GUUCUGCAAGGUGGACAAGAAGGGCAAGAACCAGUACUUCAUCGUGCCCAUCUAC |
| | | GCCUGGCAGGUGGCCGAGAACAUCCUGCCCGACAUCGACUGCAAGGGCUACCGGA |
| | | UCGACGACUCCUACACCUUCUGCUUCUCCCUGCACAAGUACGACCUGAUCGCCUU |
| | | CCAGAAGGACGAGAAGUCCAAGGUGGAGUUCGCCUACUACAUCAACUGCGACUCC |
| | | UCCAACGGCCGGUUCUACCUGGCCUGGCACGACAAGGGCUCCAAGGAGCAGCAGU |
| | | UCCGGAUCUCCACCCAGAACCUGGUGCUGAUCCAGAAGUACCAGGUGAACGAGCU |
| | | GGGCAAGGAGAUCCGGCCCUGCCGGCUGAAGAAGCGGCCCCCCGUGCGGUAGCUA |
| | | GCACCAGCCUCAAGAACACCCGAAUGGAGUCUCUAAGCUACAUAAUACCAACUUA |
| | | CACUUUACAAAAUGUUGUCCCCCAAAAUGUAGCCAUUCGUAUCUGCUCCUAAUAA |
| | | AAAGAAAGUUUCUUCACAUUCUCAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAA |
| 702 | Exemplary | GGGUCCCGCAGUCGGCGUCCAGCGGCUCUGCUUGUUCGUGUGUGUGUCGUUGCAG |
| | sequence | GCCUUAUUCGGAUCCGCCACCAUGGCAGCAUUCAAGCCGAACUCGAUCAACUACA |
| | encoding | UCCUGGGACUGGACAUCGGAAUCGCAUCGGUCGGAUGGGCAAUGGUCGAAAUCGA |
| | Nme1Cas9 | CGAAGAAGAAAACCCGAUCAGACUGAUCGACCUGGGAGUCAGAGUCUUCGAAAGA |
| | (mRNA AB) | GCAGAAGUCCCGAAGACAGGAGACUCGCUGGCAAUGGCAAGAAGACUGGCAAGAU |
| | | CGGUCAGAAGACUGACAAGAAGAAGAGCACACAGACUGCUGAGAACAAGAAGACU |
| | | GCUGAAGAGAGAAGGAGUCCUGCAGGCAGCAAACUUCGACGAAAACGGACUGAUC |
| | | AAGUCGCUGCCGAACACACCGUGGCAGCUGAGAGCAGCAGCACUGGACAGAAAGC |
| | | UGACACCGCUGGAAUGGUCGGCAGUCCUGCUGCACCUGAUCAAGCACAGAGGAUA |
| | | CCUGUCGCAGAGAAAGAACGAAGGAGAAACAGCAGACAAGGAACUGGGAGCACUG |
| | | CUGAAGGGAGUCGCAGGAAACGCACACGCACUGCAGACAGGAGACUUCAGAACAC |
| | | CGGCAGAACUGGCACUGAACAAGUUCGAAAAGGAAUCGGGACACAUCAGAAACCA |
| | | GAGAUCGGACUACUCGCACACAUUCUCGAGAAAGGACCUGCAGGCAGAACUGAUC |
| | | CUGCUGUUCGAAAAGCAGAAGGAAUUCGGAAACCCGCACGUCUCGGGAGGACUGA |
| | | AGGAAGGAAUCGAAACACUGCUGAUGACACAGAGACCGGCACUGUCGGGAGACGC |
| | | AGUCCAGAAGAUGCUGGGACACUGCACAUUCGAACCGGCAGAACCGAAGGCAGCA |
| | | AAGAACACAUACACAGCAGAAAGAUUCAUCUGGCUGACAAAGCUGAACAACCUGA |
| | | GAAUCCUGGAACAGGGAUCGGAAAGACCGCUGACAGACACAGAAAGAGCAACACU |
| | | GAUGGACGAACCGUACAGAAAGUCGAAGCUGACAUACGCACAGGCAAGAAAGCUG |
| | | CUGGGACUGGAAGACACAGCAUUCUUCAAGGGACUGAGAUACGGAAAGGACAACG |
| | | CAGAAGCAUCGACACUGAUGGAAAUGAAGGCAUACCACGCAAUCUCGAGAGCACU |
| | | GGAAAAGGAAGGACUGAAGGACAAGAAGUCGCCGCUGAACCUGUCGCCGGAACUG |
| | | CAGGACGAAAUCGGAACAGCAUUCUCGCUGUUCAAGACAGACGAAGACAUCACAG |
| | | GAAGACUGAAGGACAGAAUCCAGCCGGAAAUCCUGGAAGCACUGCUGAAGCACAU |
| | | CUCGUUCGACAAGUUCGUCCAGAUCUCGCUGAAGGCACUGAGAAGAAUCGUCCCG |
| | | CUGAUGGAACAGGGAAAGAGAUACGACGAAGCAUGCGCAGAAAUCUACGGAGACC |
| | | ACUACGGAAAGAAGAACACAGAAGAAAAGAUCUACCUGCCGCCGAUCCCGGCAGA |
| | | CGAAAUCAGAAACCCGGUCGUCCUGAGAGCACUGUCGCAGGCAAGAAAGGUCAUC |
| | | AACGGAGUCGUCAGAAGAUACGGAUCGCCGGCAAGAAUCCACAUCGAAACAGCAA |
| | | GAGAAGUCGGAAAGUCGUUCAAGGACAGAAAGGAAAUCGAAAAGAGACAGGAAGA |
| | | AAACAGAAAGGACAGAGAAAAGGCAGCAGCAAAGUUCAGAGAAUACUUCCCGAAC |
| | | UUCGUCGGAGAACCGAAGUCGAAGGACAUCCUGAAGCUGAGACUGUACGAACAGC |
| | | AGCACGGAAAGUGCCUGUACUCGGGAAAGGAAAUCAACCUGGGAAGACUGAACGA |
| | | AAAGGGAUACGUCGAAAUCGACCACGCACUGCCGUUCUCGAGAACAUGGGACGAC |
| | | UCGUUCAACAACAAGGUCCUGGUCCUGGGAUCGGAAAACCAGAACAAGGGAAACC |
| | | AGACACCGUACGAAUACUUCAACGGAAAGGACAACUCGAGAGAAUGGCAGGAAUU |
| | | CAAGGCAAGAGUCGAAACAUCGAGAUUCCCGAGAUCGAAGAAGCAGAGAAUCCUG |
| | | CUGCAGAAGUUCGACGAAGACGGAUUCAAGGAAAGAAACCUGAACGACACAAGAU |
| | | ACGUCAACAGAUUCCUGUGCCAGUUCGUCGCAGACAGAAUGAGACUGACAGGAAA |
| | | GGGAAAGAAGAGAGUCUUCGCAUCGAACGGACAGAUCACAAACCUGCUGAGAGGA |
| | | UUCUGGGGACUGAGAAAGGUCAGAGCAGAAAACGACAGACACCACGCACUGGACG |
| | | CAGUCGUCGUCGCAUGCUCGACAGUCGCAAUGCAGCAGAAGAUCACAAGAUUCGU |
| | | CAGAUACAAGGAAAUGAACGCAUUCGACGGAAAGACAAUCGACAAGGAAACAGGA |
| | | GAAGUCCUGCACCAGAAGACACACUUCCCGCAGCCGUGGGAAUUCUUCGCACAGG |
| | | AAGUCAUGAUCAGAGUCUUCGGAAAGCCGGACGGAAAGCCGGAAUUCGAAGAAGC |
| | | AGACACACUGGAAAAGCUGAGAACACUGCUGGCAGAAAAGCUGUCGUCGAGACCG |
| | | GAAGCAGUCCACGAAUACGUCACACCGCUGUUCGUCUCGAGAGCACCGAACAGAA |
| | | AGAUGUCGGGACAGGGACACAUGGAAACAGUCAAGUCGGCAAAGAGACUGGACGA |
| | | AGGAGUCUCGGUCCUGAGAGUCCCGCUGACACAGCUGAAGCUGAAGGACCUGGAA |
| | | AAGAUGGUCAACAGAGAAAGAGAACCGAAGCUGUACGAAGCACUGAAGGCAAGAC |
| | | UGGAAGCACACAAGGACGACCCGGCAAAGGCAUUCGCAGAACCGUUCUACAAGUA |
| | | CGACAAGGCAGGAAACAGAACACAGCAGGUCAAGGCAGUCAGAGUCGAACAGGUC |
| | | CAGAAGACAGGAGUCUGGGUCAGAAACCACAACGGAAUCGCAGACAACGCAACAA |
| | | UGGUCAGAGUAGACGUCUUCGAAAAGGGAGACAAGUACUACCUGGUCCCGAUCUA |
| | | CUCGUGGCAGGUCGCAAAGGGAAUCCUGCCGGACAGAGCAGUCGUCCAGGGAAAG |
| | | GACGAAGAAGACUGGCAGCUGAUCGACGACUCGUUCAACUUCAAGUUCUCGCUGC |
| | | ACCCGAACGACCUGGUCGAAGUCAUCACAAAGAAGGCAAGAAUGUUCGGAUACUU |
| | | CGCAUCGUGCCACAGAGGAACAGGAAACAUCAACAUCAGAAUCCACGACCUGGAC |
| | | CACAAGAUCGGAAAGAACGGAAUCCUGGAAGGAAUCGGAGUCAAGACAGCACUGU |
| | | CGUUCCAGAAGUACCAGAUCGACGAACUGGGAAAGGAAAUCAGACCGUGCAGACU |
| | | GAAGAAGAGACCGCCGGUCAGAUCCGGAAAGAGAACAGCAGACGGAUCGGAAUUC |
| | | GAAUCGCCGAAGAAGAAGAGAAAGGUCGAAUGAUAGCUAGCCAUCACAUUUAAAA |
| | | GCAUCUCAGCCUACCAUGAGAAUAAGAGAAAGAAAAUGAAGAUCAAUAGCUUAUU |
| | | CAUCUCUUUUUCUUUUUCGUUGGUGUAAAGCCAACACCCUGUCUAAAAAACAUAA |
| | | AUUUCUUUAAUCAUUUUGCCUCUUUUCUCUGUGCUUCAAUUAAUAAAAAAUGGAA |
| | | AGAACCUCGAGAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAUCUAG |
| 703 | Exemplary | GGGAAGCUCAGAAUAAACGCUCAACUUUGGCCGGAUCUGCCACCAUGGACGGCUC |
| | sequence | CGGCGGCGGCUCCCCCAAGAAGAAGCGGAAGGUGGAGGACAAGCGGCCCGCCGCC |
| | encoding | ACCAAGAAGGCCGGCCAGGCCAAGAAGAAGAAGGGCGGCUCCGGCGGCGGCGCCG |
| | Nme2Cas9 | CCUUCAAGCCCAACCCCAUCAACUACAUCCUGGGCCUGGACAUCGGCAUCGCCUC |
| | with | CGUGGGCUGGGCCAUGGUGGAGAUCGACGAGGAGGAGAACCCCAUCCGGCUGAUC |
| | HiBiT tag | GACCUGGGCGUGCGGGUGUUCGAGCGGGCCGAGGUGCCCAAGACCGGCGACUCCC |
| | (mRNA V) | UGGCCAUGGCCCGGCGGCUGGCCCGGUCCGUGCGGCGGCUGACCCGGCGGCGGGC |
| | | CCACCGGCUGCUGCGGGCCCGGCGGCUGCUGAAGCGGGAGGGCGUGCUGCAGGCC |
| | | GCCGACUUCGACGAGAACGGCCUGAUCAAGUCCCUGCCCAACACCCCCUGGCAGC |
| | | UGCGGGCCGCCGCCCUGGACCGGAAGCUGACCCCCCUGGAGUGGUCCGCCGUGCU |
| | | GCUGCACCUGAUCAAGCACCGGGGCUACCUGUCCCAGCGGAAGAACGAGGGCGAG |
| | | ACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGA |
| | | GAAGGAGUCCGGCCACAUCCGGAACCAGCGGGGCGACUACUCCCACACCUUCUCC |
| | | CGGAAGGACCUGCAGGCCGAGCUGAUCCUGCUGUUCGAGAAGCAGAAGGAGUUCG |
| | | GCAACCCCCACGUGUCCGGCGGCCUGAAGGAGGGCAUCGAGACCCUGCUGAUGAC |
| | | CCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCA |
| | | UCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAG |
| | | CUGACCUACGCCCAGGCCCGGAAGCUGCUGGGCCUGGAGGACACCGCCUUCUUCA |
| | | AGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAG |
| | | UCCCCCCUGAACCUGUCCUCCGAGCUGCAGGACGAGAUCGGCACCGCCUUCUCCC |
| | | UGUUCAAGACCGACGAGGACAUCACCGGCCGGCUGAAGGACCGGGUGCAGCCCGA |
| | | GAUCCUGGAGGCCCUGCUGAAGCACAUCUCCUUCGACAAGUUCGUGCAGAUCUCC |
| | | CUGAAGGCCCUGCGGCGGAUCGUGCCCCUGAUGGAGCAGGGCAAGCGGUACGACG |
| | | AGGCCUGCGCCGAGAUCUACGGCGACCACUACGGCAAGAAGAACACCGAGGAGAA |
| | | GAUCUACCUGCCCCCCAUCCCCGCCGACGAGAUCCGGAACCCCGUGGUGCUGCGG |
| | | GCCCUGUCCCAGGCCCGGAAGGUGAUCAACGGCGUGGUGCGGCGGUACGGCUCCC |
| | | CCGCCCGGAUCCACAUCGAGACCGCCCGGGAGGUGGGCAAGUCCUUCAAGGACCG |
| | | GAAGGAGAUCGAGAAGCGGCAGGAGGAGAACCGGAAGGACCGGGAGAAGGCCGCC |
| | | GCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCC |
| | | CUGCCCUUCUCCCGGACCUGGGACGACUCCUUCAACAACAAGGUGCUGGUGCUGG |
| | | GCUCCGAGAACCAGAACAAGGGCAACCAGACCCCCUACGAGUACUUCAACGGCAA |
| | | GGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCA |
| | | AGGAGUGCAACCUGAACGACACCCGGUACGUGAACCGGUUCCUGUGCCAGUUCGU |
| | | GGCCGACCACAUCCUGCUGACCGGCAAGGGCAAGCGGCGGGUGUUCGCCUCCAAC |
| | | GGCCAGAUCACCAACCUGCUGCGGGGCUUCUGGGGCCUGCGGAAGGUGCGGGCCG |
| | | AGAACGACCGGCACCACGCCCUGGACGCCGUGGUGGUGGCCUGCUCCACCGUGGC |
| | | CAUGCAGCAGAAGAUCACCCGGUUCGUGCGGUACAAGGAGAUGAACGCCUUCGAC |
| | | GGCAAGACCAUCGACAAGGAGACCGGCAAGGUGCUGCACCAGAAGACCCACUUCC |
| | | CCCAGCCCUGGGAGUUCUUCGCCCAGGAGGUGAUGAUCCGGGUGUUCGGCAAGCC |
| | | CGACGGCAAGCCCGAGUUCGAGGAGGCCGACACCCCCGAGAAGCUGCGGACCCUG |
| | | CUGGCCGAGAAGCUGUCCUCCCGGCCCGAGGCCGUGCACGAGUACGUGACCCCCC |
| | | UGUUCGUGUCCCGGGCCCCCAACCGGAAGAUGUCCGGCGCCCACAAGGACACCCU |
| | | GCGGUCCGCCAAGCGGUUCGUGAAGCACAACGAGAAGAUCUCCGUGAAGCGGGUG |
| | | UGGCUGACCGAGAUCAAGCUGGCCGACCUGGAGAACAUGGUGAACUACAAGAACG |
| | | GCCGGGAGAUCGAGCUGUACGAGGCCCUGAAGGCCCGGCUGGAGGCCUACGGCGG |
| | | CAACGCCAAGCAGGCCUUCGACCCCAAGGACAACCCCUUCUACAAGAAGGGCGGC |
| | | CAGCUGGUGAAGGCCGUGCGGGUGGAGAAGACCCAGGAGUCCGGCGUGCUGCUGA |
| | | ACAAGAAGAACGCCUACACCAUCGCCGACAACGGCGACAUGGUGCGGGUGGACGU |
| | | GUUCUGCAAGGUGGACAAGAAGGGCAAGAACCAGUACUUCAUCGUGCCCAUCUAC |
| | | GCCUGGCAGGUGGCCGAGAACAUCCUGCCCGACAUCGACUGCAAGGGCUACCGGA |
| | | UCGACGACUCCUACACCUUCUGCUUCUCCCUGCACAAGUACGACCUGAUCGCCUU |
| | | CCAGAAGGACGAGAAGUCCAAGGUGGAGUUCGCCUACUACAUCAACUGCGACUCC |
| | | UCCAACGGCCGGUUCUACCUGGCCUGGCACGACAAGGGCUCCAAGGAGCAGCAGU |
| | | UCCGGAUCUCCACCCAGAACCUGGUGCUGAUCCAGAAGUACCAGGUGAACGAGCU |
| | | GGGCAAGGAGAUCCGGCCCUGCCGGCUGAAGAAGCGGCCCCCCGUGCGGUCCGAG |
| | | UCCGCCACCCCCGAGUCCGUGUCCGGCUGGCGGCUGUUCAAGAAGAUCUCCUAGC |
| | | UAGCACCAGCCUCAAGAACACCCGAAUGGAGUCUCUAAGCUACAUAAUACCAACU |
| | | UACACUUUACAAAAUGUUGUCCCCCAAAAUGUAGCCAUUCGUAUCUGCUCCUAAU |
| | | AAAAAGAAAGUUUCUUCACAUUCUAAAAAAAAAAAAUGGAAAAAAAAAAAACGGA |
| | | AAAAAAAAAAAGGUAAAAAAAAAAAAUAUAAAAAAAAAAAACAUAAAAAAAAAAA |
| | | ACGAAAAAAAAAAAACGUAAAAAAAAAAAACUCAAAAAAAAAAAAGAUAAAAAAA |
| | | AAAAACCUAAAAAAAAAAAAUGUAAAAAAAAAAAAGGGAAAAAAAAAAAACGCAA |
| | | AAAAAAAAAACACAAAAAAAAAAAAUGCAAAAAAAAAAAAUCGAAAAAAAAAAAA |
| | | UCUAAAAAAAAAAAACGAAAAAAAAAAAACCCAAAAAAAAAAAAGACAAAAAAAA |
| | | AAAAUAGAAAAAAAAAAAAGUUAAAAAAAAAAAACUGAAAAAAAAAAAAUUUAAA |
| | | AAAAAAAAAUCUCGA |
| 704 | Exemplary | GGGAAGCUCAGAAUAAACGCUCAACUUUGGCCGGAUCUGCCACCAUGGACGGCUC |
| | sequence | CGGCGGCGGCUCCCCCAAGAAGAAGCGGAAGGUGGAGGACAAGCGGCCCGCCGCC |
| | encoding | ACCAAGAAGGCCGGCCAGGCCAAGAAGAAGAAGGGCGGCUCCGGCGGCGGCGCAG |
| | Nme1Cas9 with | CAUUCAAGCCAAACUCAAUCAAUUACAUCCUGGGACUGGACAUCGGCAUCGCAUC |
| | HiBiT tag | CGUCGGGUGGGCUAUGGUCGAAAUCGACGAGGAGGAGAACCCCAUCCGCCUGAUC |
| | (mRNA X) | GAUCUGGGCGUGCGCGUGUUUGAGAGGGCAGAGGUGCCUAAGACCGGCGACAGCC |
| | | UGGCCAUGGCACGGAGACUGGCACGCUCCGUGAGGCGCCUGACCCGGAGAAGGGC |
| | | CCACAGACUGCUGAGGACACGCCGGCUGCUGAAGAGGGAGGGCGUGCUGCAGGCC |
| | | GCCAACUUCGAUGAGAAUGGCCUGAUCAAGUCCCUGCCCAAUACCCCUUGGCAGC |
| | | UGAGGGCAGCCGCCCUGGACCGCAAGCUGACACCUCUGGAGUGGUCCGCCGUGCU |
| | | GCUGCACCUGAUCAAGCACCGGGGCUACCUGUCUCAGAGAAAGAACGAGGGCGAG |
| | | ACAGCCGAUAAGGAGCUGGGCGCCCUGCUGAAGGGAGUGGCAGGAAAUGCACACG |
| | | CCCUGCAGACCGGCGACUUUCGCACACCAGCCGAGCUGGCCCUGAACAAGUUCGA |
| | | GAAGGAGAGCGGCCACAUCCGCAAUCAGCGGUCUGACUAUAGCCACACCUUCUCC |
| | | CGGAAGGAUCUGCAGGCCGAGCUGAUCCUGCUGUUUGAGAAGCAGAAGGAGUUCG |
| | | GCAACCCACACGUGUCUGGCGGCCUGAAGGAGGGCAUCGAGACACUGCUGAUGAC |
| | | ACAGCGGCCCGCCCUGAGCGGCGACGCAGUGCAGAAGAUGCUGGGACACUGCACC |
| | | UUUGAGCCAGCCGAGCCCAAGGCCGCCAAGAAUACCUACACAGCCGAGCGGUUCA |
| | | UCUGGCUGACAAAGCUGAACAAUCUGAGGAUCCUGGAGCAGGGAAGCGAGCGCCC |
| | | ACUGACCGACACAGAGAGGGCCACCCUGAUGGAUGAGCCCUACCGCAAGUCCAAG |
| | | CUGACAUAUGCACAGGCAAGGAAGCUGCUGGGCCUGGAGGACACCGCCUUCUUUA |
| | | AGGGCCUGAGAUACGGCAAGGAUAACGCCGAGGCCUCUACACUGAUGGAGAUGAA |
| | | GGCCUAUCACGCCAUCAGCAGGGCCCUGGAGAAGGAGGGCCUGAAGGACAAGAAG |
| | | UCCCCACUGAAUCUGUCUCCCGAGCUGCAGGAUGAGAUCGGCACCGCCUUUAGCC |
| | | UGUUCAAGACCGACGAGGAUAUCACAGGCAGACUGAAGGACAGGAUCCAGCCAGA |
| | | GAUCCUGGAGGCCCUGCUGAAGCACAUCAGCUUUGAUAAGUUCGUGCAGAUCAGC |
| | | CUGAAGGCCCUGCGGAGGAUCGUGCCACUGAUGGAGCAGGGCAAGAGGUACGACG |
| | | AGGCCUGCGCCGAAAUCUACGGCGAUCACUAUGGCAAGAAGAACACAGAGGAGAA |
| | | AAUCUACCUGCCCCCUAUCCCCGCCGAUGAGAUCAGGAACCCUGUGGUGCUGCGC |
| | | GCCCUGUCUCAGGCAAGAAAAGUGAUCAACGGAGUGGUGCGCCGGUACGGCAGCC |
| | | CCGCCAGAAUCCACAUCGAGACAGCCAGGGAAGUGGGCAAGUCCUUUAAGGACAG |
| | | AAAGGAGAUCGAGAAGAGGCAGGAGGAGAACAGAAAGGAUAGGGAGAAGGCCGCC |
| | | GCCAAGUUCAGAGAGUACUUUCCUAAUUUCGUGGGCGAGCCAAAGUCCAAGGAUA |
| | | UCCUGAAGCUGAGGCUGUACGAGCAGCAGCACGGCAAGUGUCUGUAUUCUGGCAA |
| | | GGAGAUCAACCUGGGCCGCCUGAAUGAGAAGGGCUAUGUGGAGAUCGACCACGCC |
| | | CUGCCUUUUUCUCGGACCUGGGACGAUAGCUUCAACAAUAAGGUGCUGGUGCUGG |
| | | GCUCUGAGAACCAGAAUAAGGGCAACCAGACACCCUACGAGUAUUUCAACGGCAA |
| | | GGACAAUAGCCGCGAGUGGCAGGAGUUUAAGGCAAGGGUGGAGACAAGCAGGUUC |
| | | CCUCGGUCCAAGAAGCAGAGAAUCCUGCUGCAGAAGUUUGACGAGGAUGGCUUCA |
| | | AGGAGAGGAACCUGAAUGACACCCGCUACGUGAAUCGGUUUCUGUGCCAGUUCGU |
| | | GGCCGAUAGAAUGAGGCUGACCGGCAAGGGCAAGAAGAGAGUGUUUGCCUCCAAC |
| | | GGCCAGAUCACAAAUCUGCUGAGGGGCUUCUGGGGCCUGAGAAAGGUGAGGGCAG |
| | | AGAACGACAGGCACCACGCACUGGAUGCAGUGGUGGUGGCAUGUUCUACCGUGGC |
| | | CAUGCAGCAGAAGAUCACACGCUUUGUGCGGUAUAAGGAGAUGAAUGCCUUCGAC |
| | | GGCAAGACCAUCGAUAAGGAGACAGGCGAGGUGCUGCACCAGAAGACACACUUUC |
| | | CUCAGCCAUGGGAGUUCUUUGCCCAGGAAGUGAUGAUCCGGGUGUUUGGCAAGCC |
| | | UGACGGCAAGCCAGAGUUCGAGGAGGCCGAUACCCUGGAGAAGCUGAGAACACUG |
| | | CUGGCAGAGAAGCUGAGCUCCAGGCCCGAGGCAGUGCACGAGUACGUGACCCCAC |
| | | UGUUCGUGUCUAGAGCCCCCAACAGGAAGAUGAGCGGCCAGGGCCACAUGGAGAC |
| | | AGUGAAGUCCGCCAAGAGACUGGACGAGGGCGUGUCUGUGCUGAGGGUGCCUCUG |
| | | ACACAGCUGAAGCUGAAGGAUCUGGAGAAGAUGGUGAAUCGCGAGCGGGAGCCAA |
| | | AGCUGUAUGAGGCCCUGAAGGCAAGGCUGGAGGCACACAAGGACGAUCCUGCCAA |
| | | GGCCUUUGCCGAGCCAUUCUACAAGUAUGAUAAGGCCGGCAACAGAACCCAGCAG |
| | | GUGAAGGCCGUGAGGGUGGAGCAGGUGCAGAAGACAGGCGUGUGGGUGCGCAACC |
| | | ACAAUGGCAUCGCCGACAAUGCUACCAUGGUGCGGGUGGACGUGUUUGAGAAGGG |
| | | CGAUAAGUACUAUCUGGUGCCCAUCUACAGCUGGCAGGUGGCCAAGGGCAUCCUG |
| | | CCUGAUAGAGCCGUGGUGCAGGGCAAGGACGAGGAGGAUUGGCAGCUGAUCGACG |
| | | AUUCCUUCAACUUUAAGUUCUCUCUGCACCCCAAUGACCUGGUGGAAGUGAUCAC |
| | | CAAGAAGGCCAGGAUGUUUGGCUACUUCGCCUCCUGCCACCGCGGCACAGGCAAC |
| | | AUCAAUAUCCGGAUCCACGACCUGGAUCACAAGAUCGGCAAGAACGGCAUCCUGG |
| | | AGGGCAUCGGCGUGAAGACAGCCCUGAGCUUCCAGAAGUAUCAGAUCGACGAGCU |
| | | GGGCAAGGAGAUCAGACCUUGUAGGCUGAAGAAGCGCCCACCCGUGCGGUCCGAG |
| | | UCCGCCACCCCCGAGUCCGUGUCCGGCUGGCGGCUGUUCAAGAAGAUCUCCUAGC |
| | | UAGCACCAGCCUCAAGAACACCCGAAUGGAGUCUCUAAGCUACAUAAUACCAACU |
| | | UACACUUUACAAAAUGUUGUCCCCCAAAAUGUAGCCAUUCGUAUCUGCUCCUAAU |
| | | AAAAAGAAAGUUUCUUCACAUUCUAAAAAAAAAAAAUGGAAAAAAAAAAAACGGA |
| | | AAAAAAAAAAAGGUAAAAAAAAAAAAUAUAAAAAAAAAAAACAUAAAAAAAAAAA |
| | | ACGAAAAAAAAAAAACGUAAAAAAAAAAAACUCAAAAAAAAAAAAGAUAAAAAAA |
| | | AAAAACCUAAAAAAAAAAAAUGUAAAAAAAAAAAAGGGAAAAAAAAAAAACGCAA |
| | | AAAAAAAAAACACAAAAAAAAAAAAUGCAAAAAAAAAAAAUCGAAAAAAAAAAAA |
| | | UCUAAAAAAAAAAAACGAAAAAAAAAAAACCCAAAAAAAAAAAAGACAAAAAAAA |
| | | AAAAUAGAAAAAAAAAAAAGUUAAAAAAAAAAAACUGAAAAAAAAAAAAUUUAAA |
| | | AAAAAAAAAUCUCGA |
| 705 | Exemplary | GGGAAGCUCAGAAUAAACGCUCAACUUUGGCCGGAUCUGCCACCAUGGACGGCUC |
| | sequence | CGGCGGCGGCUCCCCCAAGAAGAAGCGGAAGGUGGAGGACAAGCGGCCCGCCGCC |
| | encoding | ACCAAGAAGGCCGGCCAGGCCAAGAAGAAGAAGGGCGGCUCCGGCGGCGGCGCCG |
| | Nme1Cas9 with | CCUUCAAGCCCAACUCCAUCAACUACAUCCUGGGCCUGGACAUCGGCAUCGCCUC |
| | HiBiT tag | CGUGGGCUGGGCCAUGGUGGAGAUCGACGAGGAGGAGAACCCCAUCCGGCUGAUC |
| | (mRNA Y) | GACCUGGGCGUGCGGGUGUUCGAGCGGGCCGAGGUGCCCAAGACCGGCGACUCCC |
| | | UGGCCAUGGCCCGGCGGCUGGCCCGGUCCGUGCGGCGGCUGACCCGGCGGCGGGC |
| | | CCACCGGCUGCUGCGGACCCGGCGGCUGCUGAAGCGGGAGGGCGUGCUGCAGGCC |
| | | GCCAACUUCGACGAGAACGGCCUGAUCAAGUCCCUGCCCAACACCCCCUGGCAGC |
| | | UGCGGGCCGCCGCCCUGGACCGGAAGCUGACCCCCCUGGAGUGGUCCGCCGUGCU |
| | | GCUGCACCUGAUCAAGCACCGGGGCUACCUGUCCCAGCGGAAGAACGAGGGCGAG |
| | | ACCGCCGACAAGGAGCUGGGCGCCCUGCUGAAGGGCGUGGCCGGCAACGCCCACG |
| | | CCCUGCAGACCGGCGACUUCCGGACCCCCGCCGAGCUGGCCCUGAACAAGUUCGA |
| | | GAAGGAGUCCGGCCACAUCCGGAACCAGCGGUCCGACUACUCCCACACCUUCUCC |
| | | CGGAAGGACCUGCAGGCCGAGCUGAUCCUGCUGUUCGAGAAGCAGAAGGAGUUCG |
| | | GCAACCCCCACGUGUCCGGCGGCCUGAAGGAGGGCAUCGAGACCCUGCUGAUGAC |
| | | CCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCA |
| | | UCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAG |
| | | CUGACCUACGCCCAGGCCCGGAAGCUGCUGGGCCUGGAGGACACCGCCUUCUUCA |
| | | AGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAG |
| | | UCCCCCCUGAACCUGUCCCCCGAGCUGCAGGACGAGAUCGGCACCGCCUUCUCCC |
| | | UGUUCAAGACCGACGAGGACAUCACCGGCCGGCUGAAGGACCGGAUCCAGCCCGA |
| | | GAUCCUGGAGGCCCUGCUGAAGCACAUCUCCUUCGACAAGUUCGUGCAGAUCUCC |
| | | CUGAAGGCCCUGCGGCGGAUCGUGCCCCUGAUGGAGCAGGGCAAGCGGUACGACG |
| | | AGGCCUGCGCCGAGAUCUACGGCGACCACUACGGCAAGAAGAACACCGAGGAGAA |
| | | GAUCUACCUGCCCCCCAUCCCCGCCGACGAGAUCCGGAACCCCGUGGUGCUGCGG |
| | | GCCCUGUCCCAGGCCCGGAAGGUGAUCAACGGCGUGGUGCGGCGGUACGGCUCCC |
| | | CCGCCCGGAUCCACAUCGAGACCGCCCGGGAGGUGGGCAAGUCCUUCAAGGACCG |
| | | GAAGGAGAUCGAGAAGCGGCAGGAGGAGAACCGGAAGGACCGGGAGAAGGCCGCC |
| | | GCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAA |
| | | GGAGAUCAACCUGGGCCGGCUGAACGAGAAGGGCUACGUGGAGAUCGACCACGCC |
| | | CUGCCCUUCUCCCGGACCUGGGACGACUCCUUCAACAACAAGGUGCUGGUGCUGG |
| | | GCUCCGAGAACCAGAACAAGGGCAACCAGACCCCCUACGAGUACUUCAACGGCAA |
| | | GGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCA |
| | | AGGAGCGGAACCUGAACGACACCCGGUACGUGAACCGGUUCCUGUGCCAGUUCGU |
| | | GGCCGACCGGAUGCGGCUGACCGGCAAGGGCAAGAAGCGGGUGUUCGCCUCCAAC |
| | | GGCCAGAUCACCAACCUGCUGCGGGGCUUCUGGGGCCUGCGGAAGGUGCGGGCCG |
| | | AGAACGACCGGCACCACGCCCUGGACGCCGUGGUGGUGGCCUGCUCCACCGUGGC |
| | | CAUGCAGCAGAAGAUCACCCGGUUCGUGCGGUACAAGGAGAUGAACGCCUUCGAC |
| | | GGCAAGACCAUCGACAAGGAGACCGGCGAGGUGCUGCACCAGAAGACCCACUUCC |
| | | CCCAGCCCUGGGAGUUCUUCGCCCAGGAGGUGAUGAUCCGGGUGUUCGGCAAGCC |
| | | CGACGGCAAGCCCGAGUUCGAGGAGGCCGACACCCUGGAGAAGCUGCGGACCCUG |
| | | CUGGCCGAGAAGCUGUCCUCCCGGCCCGAGGCCGUGCACGAGUACGUGACCCCCC |
| | | UGUUCGUGUCCCGGGCCCCCAACCGGAAGAUGUCCGGCCAGGGCCACAUGGAGAC |
| | | CGUGAAGUCCGCCAAGCGGCUGGACGAGGGCGUGUCCGUGCUGCGGGUGCCCCUG |
| | | ACCCAGCUGAAGCUGAAGGACCUGGAGAAGAUGGUGAACCGGGAGCGGGAGCCCA |
| | | AGCUGUACGAGGCCCUGAAGGCCCGGCUGGAGGCCCACAAGGACGACCCCGCCAA |
| | | GGCCUUCGCCGAGCCCUUCUACAAGUACGACAAGGCCGGCAACCGGACCCAGCAG |
| | | GUGAAGGCCGUGCGGGUGGAGCAGGUGCAGAAGACCGGCGUGUGGGUGCGGAACC |
| | | ACAACGGCAUCGCCGACAACGCCACCAUGGUGCGGGUGGACGUGUUCGAGAAGGG |
| | | CGACAAGUACUACCUGGUGCCCAUCUACUCCUGGCAGGUGGCCAAGGGCAUCCUG |
| | | CCCGACCGGGCCGUGGUGCAGGGCAAGGACGAGGAGGACUGGCAGCUGAUCGACG |
| | | ACUCCUUCAACUUCAAGUUCUCCCUGCACCCCAACGACCUGGUGGAGGUGAUCAC |
| | | CAAGAAGGCCCGGAUGUUCGGCUACUUCGCCUCCUGCCACCGGGGCACCGGCAAC |
| | | AUCAACAUCCGGAUCCACGACCUGGACCACAAGAUCGGCAAGAACGGCAUCCUGG |
| | | AGGGCAUCGGCGUGAAGACCGCCCUGUCCUUCCAGAAGUACCAGAUCGACGAGCU |
| | | GGGCAAGGAGAUCCGGCCCUGCCGGCUGAAGAAGCGGCCCCCCGUGCGGUCCGAG |
| | | UCCGCCACCCCCGAGUCCGUGUCCGGCUGGCGGCUGUUCAAGAAGAUCUCCUAGC |
| | | UAGCACCAGCCUCAAGAACACCCGAAUGGAGUCUCUAAGCUACAUAAUACCAACU |
| | | UACACUUUACAAAAUGUUGUCCCCCAAAAUGUAGCCAUUCGUAUCUGCUCCUAAU |
| | | AAAAAGAAAGUUUCUUCACAUUCUAAAAAAAAAAAAUGGAAAAAAAAAAAACGGA |
| | | AAAAAAAAAAAGGUAAAAAAAAAAAAUAUAAAAAAAAAAAACAUAAAAAAAAAAA |
| | | ACGAAAAAAAAAAAACGUAAAAAAAAAAAACUCAAAAAAAAAAAAGAUAAAAAAA |
| | | AAAAACCUAAAAAAAAAAAAUGUAAAAAAAAAAAAGGGAAAAAAAAAAAACGCAA |
| | | AAAAAAAAAACACAAAAAAAAAAAAUGCAAAAAAAAAAAAUCGAAAAAAAAAAAA |
| | | UCUAAAAAAAAAAAACGAAAAAAAAAAAACCCAAAAAAAAAAAAGACAAAAAAAA |
| | | AAAAUAGAAAAAAAAAAAAGUUAAAAAAAAAAAACUGAAAAAAAAAAAAUUUAAA |
| | | AAAAAAAAAUCUCGA |
| 706 | Exemplary | GGGAAGCUCAGAAUAAACGCUCAACUUUGGCCGGAUCUGCCACCAUGGACGGCUC |
| | sequence | CGGCGGCGGCUCCCCCAAGAAGAAGCGGAAGGUGGAGGACAAGCGGCCCGCCGCC |
| | encoding | ACCAAGAAGGCCGGCCAGGCCAAGAAGAAGAAGGGCGGCUCCGGCGGCGCCGCCU |
| | Nme3Cas9 | UCAAGCCCAACCCCAUCAACUACAUCCUGGGCCUGGACAUCGGCAUCGCCUCCGU |
| | with | GGGCUGGGCCAUGGUGGAGAUCGACGAGGAGGAGAACCCCAUCCGGCUGAUCGAC |
| | HiBiT tag | CUGGGCGUGQGGGUGUUCGAGCGGGCCGAGGUGCCCAAGACCGGCGACUCCCUGG |
| | (mRNA Z) | CCAUGGCCCGGCGGCUGGCCCGGUCCGUGCGGCGGCUGACCCGGCGGCGGGCCCA |
| | | CCGGCUGCUGCGGGCCCGGCGGCUGCUGAAGCGGGAGGGCGUGCUGCAGGCCGCC |
| | | GACUUCGACGAGAACGGCCUGAUCAAGUCCCUGCCCAACACCCCCUGGCAGCUGC |
| | | GGGCCGCCGCCCUGGACCGGAAGCUGACCCCCCUGGAGUGGUCCGCCGUGCUGCU |
| | | GCACCUGAUCAAGCACCGGGGCUACCUGUCCCAGCGGAAGAACGAGGGCGAGACC |
| | | GCCGACAAGGAGCUGGGCGCCCUGCUGAAGGGCGUGGCCGACAACGCCCACGCCC |
| | | UGCAGACCGGCGACUUCCGGACCCCCGCCGAGCUGGCCCUGAACAAGUUCGAGAA |
| | | GGAGUGCGGCCACAUCCGGAACCAGCGGGGCGACUACUCCCACACCUUCUCCCGG |
| | | AAGGACCUGCAGGCCGAGCUGAACCUGCUGUUCGAGAAGCAGAAGGAGUUCGGCA |
| | | ACCCCCACGUGUCCGGCGGCCUGAAGGAGGGCAUCGAGACCCUGCUGAUGACCCA |
| | | GCGGCCCGCCCUGUCCGGCGACGCCGUGCAGAAGAUGCUGGGCCACUGCACCUUC |
| | | GAGCCCGCCGAGCCCAAGGCCGCCAAGAACACCUACACCGCCGAGCGGUUCAUCU |
| | | GGCUGACCAAGCUGAACAACCUGCGGAUCCUGGAGCAGGGCUCCGAGCGGCCCCU |
| | | GACCGACACCGAGCGGGCCACCCUGAUGGACGAGCCCUACCGGAAGUCCAAGCUG |
| | | ACCUACGCCCAGGCCCGGAAGCUGCUGUCCCUGGAGGACACCGCCUUCUUCAAGG |
| | | GCCUGCGGUACGGCAAGGACAACGCCGAGGCCUCCACCCUGAUGGAGAUGAAGGC |
| | | CUACCACACCAUCUCCCGGGCCCUGGAGAAGGAGGGCCUGAAGGACAAGAAGUCC |
| | | CCCCUGAACCUGUCCCCCGAGCUGCAGGACGAGAUCGGCACCGCCUUCUCCCUGU |
| | | UCAAGACCGACGAGGACAUCACCGGCCGGCUGAAGGACCGGAUCCAGCCCGAGAU |
| | | CCUGGAGGCCCUGCUGAAGCACAUCUCCUUCGACAAGUUCGUGCAGAUCUCCCUG |
| | | AAGGCCCUGCGGCGGAUCGUGCCCCUGAUGGAGCAGGGCAAGCGGUACGACGAGG |
| | | CCUGCGCCGAGAUCUACGGCGACCACUACGGCAAGAAGAACACCGAGGAGAAGAU |
| | | CUACCUGCCCCCCAUCCCCGCCGACGAGAUCCGGAACCCCGUGGUGCUGCGGGCC |
| | | CUGUCCCAGGCCCGGAAGGUGAUCAACGGCGUGGUGCGGCGGUACGGCUCCCCCG |
| | | CCCGGAUCCACAUCGAGACCGCCCGGGAGGUGGGCAAGUCCUUCAAGGACCGGAA |
| | | GGAGAUCGAGAAGCGGCAGGAGGAGAACCGGAAGGACCGGGAGAAGGCCGCCGCC |
| | | AAGUUCCGGGAGUACUUCCCCAACUUCGUGGGCGAGCCCAAGUCCAAGGACAUCC |
| | | UGAAGCUGCGGCUGUACGAGCAGCAGCACGGCAAGUGCCUGUACUCCGGCAAGGA |
| | | GAUCAACCUGGGCCGGCUGAACGAGAAGGGCUACGUGGAGAUCGACCACGCCCUG |
| | | CCCUUCUCCCGGACCUGGGACGACUCCUUCAACAACAAGGUGCUGGUGCUGGGCU |
| | | CCGAGAACCAGAACAAGGGCAACCAGACCCCCUACGAGUACUUCAACGGCAAGGA |
| | | CAACUCCCGGGAGUGGCAGGAGUUCAAGGCCCGGGUGGAGACCUCCCGGUUCCCC |
| | | CGGUCCAAGAAGCAGCGGAUCCUGCUGCAGAAGUUCGACGAGGACGGCUUCAAGG |
| | | AGCGGAACCUGAACGACACCCGGUACGUGAACCGGUUCCUGUGCCAGUUCGUGGC |
| | | CGACCGGAUGCGGCUGACCGGCAAGGGCAAGAAGCGGGUGUUCGCCUCCAACGGC |
| | | CAGAUCACCAACCUGCUGCGGGGCUUCUGGGGCCUGCGGAAGGUGCGGGCCGAGA |
| | | ACGACCGGCACCACGCCCUGGACGCCGUGGUGGUGGCCUGCUCCACCGUGGCCAU |
| | | GCAGCAGAAGAUCACCCGGUUCGUGCGGUACAAGGAGAUGAACGCCUUCGACGGC |
| | | AAGACCAUCGACAAGGAGACCGGCGAGGUGCUGCACCAGAAGACCCACUUCCCCC |
| | | AGCCCUGGGAGUUCUUCGCCCAGGAGGUGAUGAUCCGGGUGUUCGGCAAGCCCGA |
| | | CGGCAAGCCCGAGUUCGAGGAGGCCGACACCCCCGAGAAGCUGCGGACCCUGCUG |
| | | GCCGAGAAGCUGUCCUCCCGGCCCGAGGCCGUGCACGAGUACGUGACCCCCCUGU |
| | | UCGUGUCCCGGGCCCCCAACCGGAAGAUGUCCGGCCAGGGCCACAUGGAGACCGU |
| | | GAAGUCCGCCAAGCGGCUGGACGAGGGCGUGUCCGUGCUGCGGGUGCCCCUGACC |
| | | CAGCUGAAGCUGAAGGACCUGGAGAAGAUGGUGAACCGGGAGCGGGAGCCCAAGC |
| | | UGUACGAGGCCCUGAAGGCCCGGCUGGAGGCCCACAAGGACGACCCCGCCAAGGC |
| | | CUUCGCCGAGCCCUUCUACAAGUACGACAAGGCCGGCAACCGGACCCAGCAGGUG |
| | | AAGGCCGUGCGGGUGGAGCAGGUGCAGAAGACCGGCGUGUGGGUGCGGAACCACA |
| | | ACGGCAUCGCCGACAACGCCACCAUGGUGCGGGUGGACGUGUUCGAGAAGGGCGA |
| | | CAAGUACUACCUGGUGCCCAUCUACUCCUGGCAGGUGGCCAAGGGCAUCCUGCCC |
| | | GACCGGGCCGUGGUGGCCUACGCCGACGAGGAGGACUGGACCGUGAUCGACGAGU |
| | | CCUUCCGGUUCAAGUUCGUGCUGUACUCCAACGACCUGAUCAAGGUGCAGCUGAA |
| | | GAAGGACUCCUUCCUGGGCUACUUCUCCGGCCUGGACCGGGCCACCGGCGCCAUC |
| | | UCCCUGCGGGAGCACGACCUGGAGAAGUCCAAGGGCAAGGACGGCAUGCACCGGA |
| | | UCGGCGUGAAGACCGCCCUGUCCUUCCAGAAGUACCAGAUCGACGAGAUGGGCAA |
| | | GGAGAUCCGGCCCUGCCGGCUGAAGAAGCGGCCCCCCGUGCGGUCCGAGUCCGCC |
| | | ACCCCCGAGUCCGUGUCCGGCUGGCGGCUGUUCAAGAAGAUCUCCUAGCUAGCAC |
| | | CAGCCUCAAGAACACCCGAAUGGAGUCUCUAAGCUACAUAAUACCAACUUACACU |
| | | UUACAAAAUGUUGUCCCCCAAAAUGUAGCCAUUCGUAUCUGCUCCUAAUAAAAAG |
| | | AAAGUUUCUUCACAUUCUCUCGAGAAAAAAAAAAAAUGGAAAAAAAAAAAACGGA |
| | | AAAAAAAAAAAGGTAAAAAAAAAAAAUAUAAAAAAAAAAAACAUAAAAAAAAAAA |
| | | ACGAAAAAAAAAAAACGUAAAAAAAAAAAACUCAAAAAAAAAAAAGAUAAAAAAA |
| | | AAAAACCUAAAAAAAAAAAAUGUAAAAAAAAAAAAGGGAAAAAAAAAAAACGCAA |
| | | AAAAAAAAAACACAAAAAAAAAAAAUGCAAAAAAAAAAAAUCGAAAAAAAAAAAA |
| | | UCUAAAAAAAAAAAACGAAAAAAAAAAAACCCAAAAAAAAAAAAGACAAAAAAAA |
| | | AAAAUAGAAAAAAAAAAAAGUUAAAAAAAAAAAACUGAAAAAAAAAAAAUUUAAA |
| | | AAAAAAAAAUCUAG |
| 707 | Exemplary amino | MDGSGGGSPKKKRKVEDKRPAATKKAGQAKKKKGGSGGGAAFKPNPINYILGLDI |
| | acid sequence | GIASVGWAMVEIDEEENPIRLIDLGVRVFERAEVPKTGDSLAMARRLARSVRRLT |
| | for Nme2Cas9 | RRRAHRLLRARRLLKREGVLQAADEDENGLIKSLPNTPWQLRAAALDRKLTPLEW |
| | (mRNA AA amino | SAVLLHLIKHRGYLSQRKNEGETADKELGALLKGVANNAHALQTGDFRTPAELAL |
| | acid) | NKFEKESGHIRNQRGDYSHTFSRKDLQAELILLFEKQKEFGNPHVSGGLKEGIET |
| | | LLMTQRPALSGDAVQKMLGHCTFEPAEPKAAKNTYTAERFIWLTKLNNLRILEQG |
| | | SERPLTDTERATLMDEPYRKSKLTYAQARKLLGLEDTAFFKGLRYGKDNAEASTL |
| | | MEMKAYHAISRALEKEGLKDKKSPLNLSSELQDEIGTAFSLEKTDEDITGRLKDR |
| | | VQPEILEALLKHISFDKFVQISLKALRRIVPLMEQGKRYDEACAEIYGDHYGKKN |
| | | TEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCL |
| | | YSGKEINLVRLNEKGYVEIDHALPFSRTWDDSFNNKVLVLGSENQNKGNQTPYEY |
| | | ENGKDNSREWQEFKARVETSRFPRSKKQRILLQKFDEDGEKECNLNDTRYVNRFL |
| | | CQFVADHILLTGKGKRRVFASNGQITNLLRGFWGLRKVRAENDRHHALDAVVVAC |
| | | STVAMQQKITRFVRYKEMNAFDGKTIDKETGKVLHQKTHFPQPWEFFAQEVMIRV |
| | | FGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLE |
| | | AYGGNAKQAFDPKDNPFYKKGGQLVKAVRVEKTQESGVLLNKKNAYTIADNGDMV |
| | | RVDVFCKVDKKGKNQYFIVPIYAWQVAENILPDIDCKGYRIDDSYTFCFSLHKYD |
| | | LIAFQKDEKSKVEFAYYINCDSSNGRFYLAWHDKGSKEQQFRISTQNLVLIQKYQ |
| | | VNELGKEIRPCRLKKRPPVR* |
| 708 | Exemplary amino | MAAFKPNSINYILGLDIGIASVGWAMVEIDEEENPIRLIDLGVRVFERAEVPKTG |
| | acid sequence | DSLAMARRLARSVRRLTRRRAHRLLRTRRLLKREGVLQAANFDENGLIKSLPNTP |
| | for Nme1Cas9 | WQLRAAALDRKLTPLEWSAVLLHLIKHRGYLSQRKNEGETADKELGALLKGVAGN |
| | (mRNA BB amino | AHALQTGDFRTPAELALNKFEKESGHIRNQRSDYSHTFSRKDLQAELILLFEKQK |
| | acid) | EFGNPHVSGGLKEGIETLLMTQRPALSGDAVQKMLGHCTFEPAEPKAAKNTYTAE |
| | | RFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSPELQDEIGTA |
| | | FSLEKTDEDITGRLKDRIQPEILEALLKHISFDKFVQISLKALRRIVPLMEQGKR |
| | | YDEACAEIYGDHYGKKNTEEKIYLPPIPADEIRNPVVLRALSQARKVINGVVRRY |
| | | GSPARIHIETAREVGKSFKDRKEIEKRQEENRKDREKAAAKFREYFPNFVGEPKS |
| | | KDILKLRLYEQQHGKCLYSGKEINLGRLNEKGYVEIDHALPFSRTWDDSENNKVL |
| | | VLGSENQNKGNQTPYEYENGKDNSREWQEFKARVETSRFPRSKKQRILLQKFDED |
| | | GEKERNLNDTRYVNRFLCQFVADRMRLTGKGKKRVFASNGQITNLLRGFWGLRKV |
| | | RAENDRHHALDAVVVACSTVAMQQKITRFVRYKEMNAFDGKTIDKETGEVLHQKT |
| | | HFPQPWEFFAQEVMIRVFGKPDGKPEFEEADTLEKLRTLLAEKLSSRPEAVHEYV |
| | | TPLFVSRAPNRKMSGQGHMETVKSAKRLDEGVSVLRVPLTQLKLKDLEKMVNRER |
| | | EPKLYEALKARLEAHKDDPAKAFAEPFYKYDKAGNRTQQVKAVRVEQVQKTGVWV |
| | | RNHNGIADNATMVRVDVFEKGDKYYLVPIYSWQVAKGILPDRAVVQGKDEEDWQL |
| | | IDDSENEKFSLHPNDLVEVITKKARMFGYFASCHRGTGNINIRIHDLDHKIGKNG |
| | | ILEGIGVKTALSFQKYQIDELGKEIRPCRLKKRPPVRSGKRTADGSEFESPKKKR |
| | | KVE* |
| 709 | Exemplary amino | MDGSGGGSPKKKRKVEDKRPAATKKAGQAKKKKGGSGGGAAFKPNPINYILGLDI |
| | acid sequence | GIASVGWAMVEIDEEENPIRLIDLGVRVFERAEVPKTGDSLAMARRLARSVRRLT |
| | for Nme2Cas9 | RRRAHRLLRARRLLKREGVLQAADEDENGLIKSLPNTPWQLRAAALDRKLTPLEW |
| | with HiBiT tag | SAVLLHLIKHRGYLSQRKNEGETADKELGALLKGVANNAHALQTGDERTPAELAL |
| | (mRNA V amino | NKFEKESGHIRNQRGDYSHTFSRKDLQAELILLFEKQKEFGNPHVSGGLKEGIET |
| | acid) | LLMTQRPALSGDAVQKMLGHCTFEPAEPKAAKNTYTAERFIWLTKLNNLRILEQG |
| | | SERPLTDTERATLMDEPYRKSKLTYAQARKLLGLEDTAFFKGLRYGKDNAEASTL |
| | | MEMKAYHAISRALEKEGLKDKKSPLNLSSELQDEIGTAFSLFKTDEDITGRLKDR |
| | | VQPEILEALLKHISFDKFVQISLKALRRIVPLMEQGKRYDEACAEIYGDHYGKKN |
| | | TEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCL |
| | | YSGKEINLVRLNEKGYVEIDHALPESRTWDDSENNKVLVLGSENQNKGNQTPYEY |
| | | ENGKDNSREWQEFKARVETSRFPRSKKQRILLQKFDEDGEKECNLNDTRYVNRFL |
| | | CQFVADHILLTGKGKRRVFASNGQITNLLRGFWGLRKVRAENDRHHALDAVVVAC |
| | | STVAMQQKITRFVRYKEMNAFDGKTIDKETGKVLHQKTHFPQPWEFFAQEVMIRV |
| | | FGKPDGKPEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYEALKARLE |
| | | AYGGNAKQAFDPKDNPFYKKGGQLVKAVRVEKTQESGVLLNKKNAYTIADNGDMV |
| | | RVDVFCKVDKKGKNQYFIVPIYAWQVAENILPDIDCKGYRIDDSYTFCFSLHKYD |
| | | LIAFQKDEKSKVEFAYYINCDSSNGRFYLAWHDKGSKEQQFRISTQNLVLIQKYQ |
| | | VNELGKEIRPCRLKKRPPVRSESATPESVSGWRLFKKIS* |
| 710 | Exemplary amino | MDGSGGGSPKKKRKVEDKRPAATKKAGQAKKKKGGSGGGAAFKPNSINYILGLDI |
| | acid sequence | GIASVGWAMVEIDEEENPIRLIDLGVRVFERAEVPKTGDSLAMARRLARSVRRLT |
| | for Nme1Cas9 | RRRAHRLLRTRRLLKREGVLQAANEDENGLIKSLPNTPWQLRAAALDRKLTPLEW |
| | with HiBiT tag | SAVLLHLIKHRGYLSQRKNEGETADKELGALLKGVAGNAHALQTGDERTPAELAL |
| | (mRNA X amino | NKFEKESGHIRNQRSDYSHTFSRKDLQAELILLFEKQKEFGNPHVSGGLKEGIET |
| | acid) | LLMTQRPALSGDAVQKMLGHCTFEPAEPKAAKNTYTAERFIWLTKLNNLRILEQG |
| | | SERPLTDTERATLMDEPYRKSKLTYAQARKLLGLEDTAFFKGLRYGKDNAEASTL |
| | | MEMKAYHAISRALEKEGLKDKKSPLNLSPELQDEIGTAFSLFKTDEDITGRLKDR |
| | | IQPEILEALLKHISFDKFVQISLKALRRIVPLMEQGKRYDEACAEIYGDHYGKKN |
| | | TEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCL |
| | | YSGKEINLGRLNEKGYVEIDHALPFSRTWDDSFNNKVLVLGSENQNKGNQTPYEY |
| | | ENGKDNSREWQEFKARVETSRFPRSKKQRILLQKFDEDGEKERNLNDTRYVNRFL |
| | | CQFVADRMRLTGKGKKRVFASNGQITNLLRGFWGLRKVRAENDRHHALDAVVVAC |
| | | STVAMQQKITRFVRYKEMNAFDGKTIDKETGEVLHQKTHFPQPWEFFAQEVMIRV |
| | | FGKPDGKPEFEEADTLEKLRTLLAEKLSSRPEAVHEYVTPLFVSRAPNRKMSGQG |
| | | HMETVKSAKRLDEGVSVLRVPLTQLKLKDLEKMVNREREPKLYEALKARLEAHKD |
| | | DPAKAFAEPFYKYDKAGNRTQQVKAVRVEQVQKTGVWVRNHNGIADNATMVRVDV |
| | | FEKGDKYYLVPIYSWQVAKGILPDRAVVQGKDEEDWQLIDDSFNFKESLHPNDLV |
| | | EVITKKARMFGYFASCHRGTGNINIRIHDLDHKIGKNGILEGIGVKTALSFQKYQ |
| | | IDELGKEIRPCRLKKRPPVRSESATPESVSGWRLFKKIS* |
| 711 | Exemplary amino | MDGSGGGSPKKKRKVEDKRPAATKKAGQAKKKKGGSGGGAAFKPNSINYILGLDI |
| | acid sequence | GIASVGWAMVEIDEEENPIRLIDLGVRVFERAEVPKTGDSLAMARRLARSVRRLT |
| | for Nme1Cas9 | RRRAHRLLRTRRLLKREGVLQAANEDENGLIKSLPNTPWQLRAAALDRKLTPLEW |
| | with HiBiT tag | SAVLLHLIKHRGYLSQRKNEGETADKELGALLKGVAGNAHALQTGDERTPAELAL |
| | (mRNA Y amino | NKFEKESGHIRNQRSDYSHTFSRKDLQAELILLFEKQKEFGNPHVSGGLKEGIET |
| | acid) | LLMTQRPALSGDAVQKMLGHCTFEPAEPKAAKNTYTAERFIWLTKLNNLRILEQG |
| | | SERPLTDTERATLMDEPYRKSKLTYAQARKLLGLEDTAFFKGLRYGKDNAEASTL |
| | | MEMKAYHAISRALEKEGLKDKKSPLNLSPELQDEIGTAFSLFKTDEDITGRLKDR |
| | | IQPEILEALLKHISFDKFVQISLKALRRIVPLMEQGKRYDEACAEIYGDHYGKKN |
| | | TEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCL |
| | | YSGKEINLGRLNEKGYVEIDHALPESRTWDDSENNKVLVLGSENQNKGNQTPYEY |
| | | ENGKDNSREWQEFKARVETSRFPRSKKQRILLQKFDEDGEKERNLNDTRYVNRFL |
| | | CQFVADRMRLTGKGKKRVFASNGQITNLLRGFWGLRKVRAENDRHHALDAVVVAC |
| | | STVAMQQKITRFVRYKEMNAFDGKTIDKETGEVLHQKTHFPQPWEFFAQEVMIRV |
| | | FGKPDGKPEFEEADTLEKLRTLLAEKLSSRPEAVHEYVTPLFVSRAPNRKMSGQG |
| | | HMETVKSAKRLDEGVSVLRVPLTQLKLKDLEKMVNREREPKLYEALKARLEAHKD |
| | | DPAKAFAEPFYKYDKAGNRTQQVKAVRVEQVQKTGVWVRNHNGIADNATMVRVDV |
| | | FEKGDKYYLVPIYSWQVAKGILPDRAVVQGKDEEDWQLIDDSFNFKFSLHPNDLV |
| | | EVITKKARMFGYFASCHRGTGNINIRIHDLDHKIGKNGILEGIGVKTALSFQKYQ |
| | | IDELGKEIRPCRLKKRPPVRSESATPESVSGWRLFKKIS* |
| 712 | Exemplary amino | MDGSGGGSPKKKRKVEDKRPAATKKAGQAKKKKGGSGGAAFKPNPINYILGLDIG |
| | acid sequence | IASVGWAMVEIDEEENPIRLIDLGVRVFERAEVPKTGDSLAMARRLARSVRRLTR |
| | for Nme3Cas9 | RRAHRLLRARRLLKREGVLQAADFDENGLIKSLPNTPWQLRAAALDRKLTPLEWS |
| | with HiBiT tag | AVLLHLIKHRGYLSQRKNEGETADKELGALLKGVADNAHALQTGDFRTPAELALN |
| | (mRNA Z amino | KFEKECGHIRNQRGDYSHTFSRKDLQAELNLLFEKQKEFGNPHVSGGLKEGIETL |
| | acid) | LMTQRPALSGDAVQKMLGHCTFEPAEPKAAKNTYTAERFIWLTKLNNLRILEQGS |
| | | ERPLTDTERATLMDEPYRKSKLTYAQARKLLSLEDTAFFKGLRYGKDNAEASTLM |
| | | EMKAYHTISRALEKEGLKDKKSPLNLSPELQDEIGTAFSLFKTDEDITGRLKDRI |
| | | QPEILEALLKHISFDKFVQISLKALRRIVPLMEQGKRYDEACAEIYGDHYGKKNT |
| | | EEKIYLPPIPADEIRNPVVLRALSQARKVINGVVRRYGSPARIHIETAREVGKSF |
| | | KDRKEIEKRQEENRKDREKAAAKFREYFPNFVGEPKSKDILKLRLYEQQHGKCLY |
| | | SGKEINLGRLNEKGYVEIDHALPFSRTWDDSENNKVLVLGSENQNKGNQTPYEYE |
| | | NGKDNSREWQEFKARVETSRFPRSKKQRILLQKFDEDGEKERNLNDTRYVNRFLC |
| | | QFVADRMRLTGKGKKRVFASNGQITNLLRGFWGLRKVRAENDRHHALDAVVVACS |
| | | TVAMQQKITRFVRYKEMNAFDGKTIDKETGEVLHQKTHFPQPWEFFAQEVMIRVF |
| | | GKPDGKPEFEEADTPEKLRTLLAEKLSSRPEAVHEYVTPLFVSRAPNRKMSGQGH |
| | | METVKSAKRLDEGVSVLRVPLTQLKLKDLEKMVNREREPKLYEALKARLEAHKDD |
| | | PAKAFAEPFYKYDKAGNRTQQVKAVRVEQVQKTGVWVRNHNGIADNATMVRVDVF |
| | | EKGDKYYLVPIYSWQVAKGILPDRAVVAYADEEDWTVIDESFRFKFVLYSNDLIK |
| | | VQLKKDSFLGYESGLDRATGAISLREHDLEKSKGKDGMHRIGVKTALSFQKYQID |
| | | EMGKEIRPCRLKKRPPVRSESATPESVSGWRLFKKIS* |
| 713 | Exemplary open | augGACGGCUCCGGCGGCGGCUCCCCCAAGAAGAAGCGGAAGGUGGAGGACAAGC |
| | reading frame | GGCCCGCCGCCACCAAGAAGGCCGGCCAGGCCAAGAAGAAGAAGGGCGGCUCCGG |
| | for Nme2Cas9 | CGGCGGCgccgccuucaagcccaaccccaucaacuacauccugggccuggacauc |
| | (mRNA AA ORF) | ggcaucgccuccgugggcugggccaugguggagaucgacgaggaggagaacccca |
| | | uccggcugaucgaccugggcgugcggguguucgagcgggccgaggugcccaagac |
| | | cggcgacucccuggccauggcccggcggcuggcccgguccgugcggcggcugacc |
| | | cggcggcgggcccaccggcugcugcgggcccggcggcugcugaagcgggagggcg |
| | | ugcugcaggccgccgacuucgacgagaacggccugaucaagucccugcccaacac |
| | | ccccuggcagcugcgggccgccgcccuggaccggaagcugaccccccuggagugg |
| | | uccgccgugcugcugcaccugaucaagcaccggggcuaccugucccagcggaaga |
| | | acgagggcgagaccgccgacaaggagcugggcgcccugcugaagggcguggccaa |
| | | caacgcccacgcccugcagaccggcgacuuccggacccccgccgagcuggcccug |
| | | aacaaguucgagaaggaguccggccacauccggaaccagcggggcgacuacuccc |
| | | acaccuucucccggaaggaccugcaggccgagcugauccugcuguucgagaagca |
| | | gaaggaguucggcaacccccacguguccggcggccugaaggagggcaucgagacc |
| | | cugcugaugacccagcggcccgcccuguccggcgacgccgugcagaagaugcugg |
| | | gccacugcaccuucgagcccgccgagcccaaggccgccaagaacaccuacaccgc |
| | | cgagcgguucaucuggcugaccaagcugaacaaccugcggauccuggagcagggc |
| | | uccgagcggccccugaccgacaccgagcgggccacccugauggacgagcccuacc |
| | | ggaaguccaagcugaccuacgcccaggcccggaagcugcugggccuggaggacac |
| | | cgccuucuucaagggccugcgguacggcaaggacaacgccgaggccuccacccug |
| | | auggagaugaaggccuaccacgccaucucccgggcccuggagaaggagggccuga |
| | | aggacaagaaguccccccugaaccuguccuccgagcugcaggacgagaucggcac |
| | | cgccuucucccuguucaagaccgacgaggacaucaccggccggcugaaggaccgg |
| | | gugcagcccgagauccuggaggcccugcugaagcacaucuccuucgacaaguucg |
| | | ugcagaucucccugaaggcccugcggcggaucgugccccugauggagcagggcaa |
| | | gcgguacgacgaggccugcgccgagaucuacggcgaccacuacggcaagaagaac |
| | | accgaggagaagaucuaccugccccccauccccgccgacgagauccggaaccccg |
| | | uggugcugcgggcccugucccaggcccggaaggugaucaacggcguggugcggcg |
| | | guacggcucccccgcccggauccacaucgagaccgcccgggaggugggcaagucc |
| | | uucaaggaccggaaggagaucgagaagcggcaggaggagaaccggaaggaccggg |
| | | agaaggccgccgccaaguuccgggaguacuuccccaacuucgugggcgagcccaa |
| | | guccaaggacauccugaagcugcggcuguacgagcagcagcacggcaagugccug |
| | | uacuccggcaaggagaucaaccuggugcggcugaacgagaagggcuacguggaga |
| | | ucgaccacgcccugcccuucucccggaccugggacgacuccuucaacaacaaggu |
| | | gcuggugcugggcuccgagaaccagaacaagggcaaccagacccccuacgaguac |
| | | uucaacggcaaggacaacucccgggaguggcaggaguucaaggcccggguggaga |
| | | ccucccgguucccccgguccaagaagcagcggauccugcugcagaaguucgacga |
| | | ggacggcuucaaggagugcaaccugaacgacacccgguacgugaaccgguuccug |
| | | ugccaguucguggccgaccacauccugcugaccggcaagggcaagcggcgggugu |
| | | ucgccuccaacggccagaucaccaaccugcugcggggcuucuggggccugcggaa |
| | | ggugcgggccgagaacgaccggcaccacgcccuggacgccguggugguggccugc |
| | | uccaccguggccaugcagcagaagaucacccgguucgugcgguacaaggagauga |
| | | acgccuucgacggcaagaccaucgacaaggagaccggcaaggugcugcaccagaa |
| | | gacccacuucccccagcccugggaguucuucgcccaggaggugaugauccgggug |
| | | uucggcaagcccgacggcaagcccgaguucgaggaggccgacacccccgagaagc |
| | | ugcggacccugcuggccgagaagcuguccucccggcccgaggccgugcacgagua |
| | | cgugaccccccuguucgugucccgggcccccaaccggaagauguccggcgcccac |
| | | aaggacacccugcgguccgccaagcgguucgugaagcacaacgagaagaucuccg |
| | | ugaagcggguguggcugaccgagaucaagcuggccgaccuggagaacauggugaa |
| | | cuacaagaacggccgggagaucgagcuguacgaggcccugaaggcccggcuggag |
| | | gccuacggcggcaacgccaagcaggccuucgaccccaaggacaaccccuucuaca |
| | | agaagggcggccagcuggugaaggccgugcggguggagaagacccaggaguccgg |
| | | cgugcugcugaacaagaagaacgccuacaccaucgccgacaacggcgacauggug |
| | | cggguggacguguucugcaagguggacaagaagggcaagaaccaguacuucaucg |
| | | ugcccaucuacgccuggcagguggccgagaacauccugcccgacaucgacugcaa |
| | | gggcuaccggaucgacgacuccuacaccuucugcuucucccugcacaaguacgac |
| | | cugaucgccuuccagaaggacgagaaguccaagguggaguucgccuacuacauca |
| | | acugcgacuccuccaacggccgguucuaccuggccuggcacgacaagggcuccaa |
| | | ggagcagcaguuccggaucuccacccagaaccuggugcugauccagaaguaccag |
| | | gugaacgagcugggcaaggagauccggcccugccggcugaagaagcggccccccg |
| | | ugcgguag |
| 714 | Exemplary open | AUGGCAGCAUUCAAGCCGAACUCGAUCAACUACAUCCUGGGACUGGACAUCGGAA |
| | reading frame | UCGCAUCGGUCGGAUGGGCAAUGGUCGAAAUCGACGAAGAAGAAAACCCGAUCAG |
| | for Nme1Cas9 | ACUGAUCGACCUGGGAGUCAGAGUCUUCGAAAGAGCAGAAGUCCCGAAGACAGGA |
| | (mRNA AB ORF) | GACUCGCUGGCAAUGGCAAGAAGACUGGCAAGAUCGGUCAGAAGACUGACAAGAA |
| | | GAAGAGCACACAGACUGCUGAGAACAAGAAGACUGCUGAAGAGAGAAGGAGUCCU |
| | | GCAGGCAGCAAACUUCGACGAAAACGGACUGAUCAAGUCGCUGCCGAACACACCG |
| | | UGGCAGCUGAGAGCAGCAGCACUGGACAGAAAGCUGACACCGCUGGAAUGGUCGG |
| | | CAGUCCUGCUGCACCUGAUCAAGCACAGAGGAUACCUGUCGCAGAGAAAGAACGA |
| | | AGGAGAAACAGCAGACAAGGAACUGGGAGCACUGCUGAAGGGAGUCGCAGGAAAC |
| | | GCACACGCACUGCAGACAGGAGACUUCAGAACACCGGCAGAACUGGCACUGAACA |
| | | AGUUCGAAAAGGAAUCGGGACACAUCAGAAACCAGAGAUCGGACUACUCGCACAC |
| | | AUUCUCGAGAAAGGACCUGCAGGCAGAACUGAUCCUGCUGUUCGAAAAGCAGAAG |
| | | GAAUUCGGAAACCCGCACGUCUCGGGAGGACUGAAGGAAGGAAUCGAAACACUGC |
| | | UGAUGACACAGAGACCGGCACUGUCGGGAGACGCAGUCCAGAAGAUGCUGGGACA |
| | | CUGCACAUUCGAACCGGCAGAACCGAAGGCAGCAAAGAACACAUACACAGCAGAA |
| | | AGAUUCAUCUGGCUGACAAAGCUGAACAACCUGAGAAUCCUGGAACAGGGAUCGG |
| | | AAAGACCGCUGACAGACACAGAAAGAGCAACACUGAUGGACGAACCGUACAGAAA |
| | | GUCGAAGCUGACAUACGCACAGGCAAGAAAGCUGCUGGGACUGGAAGACACAGCA |
| | | UUCUUCAAGGGACUGAGAUACGGAAAGGACAACGCAGAAGCAUCGACACUGAUGG |
| | | AAAUGAAGGCAUACCACGCAAUCUCGAGAGCACUGGAAAAGGAAGGACUGAAGGA |
| | | CAAGAAGUCGCCGCUGAACCUGUCGCCGGAACUGCAGGACGAAAUCGGAACAGCA |
| | | UUCUCGCUGUUCAAGACAGACGAAGACAUCACAGGAAGACUGAAGGACAGAAUCC |
| | | AGCCGGAAAUCCUGGAAGCACUGCUGAAGCACAUCUCGUUCGACAAGUUCGUCCA |
| | | GAUCUCGCUGAAGGCACUGAGAAGAAUCGUCCCGCUGAUGGAACAGGGAAAGAGA |
| | | UACGACGAAGCAUGCGCAGAAAUCUACGGAGACCACUACGGAAAGAAGAACACAG |
| | | AAGAAAAGAUCUACCUGCCGCCGAUCCCGGCAGACGAAAUCAGAAACCCGGUCGU |
| | | CCUGAGAGCACUGUCGCAGGCAAGAAAGGUCAUCAACGGAGUCGUCAGAAGAUAC |
| | | GGAUCGCCGGCAAGAAUCCACAUCGAAACAGCAAGAGAAGUCGGAAAGUCGUUCA |
| | | AGGACAGAAAGGAAAUCGAAAAGAGACAGGAAGAAAACAGAAAGGACAGAGAAAA |
| | | GGCAGCAGCAAAGUUCAGAGAAUACUUCCCGAACUUCGUCGGAGAACCGAAGUCG |
| | | AAGGACAUCCUGAAGCUGAGACUGUACGAACAGCAGCACGGAAAGUGCCUGUACU |
| | | CGGGAAAGGAAAUCAACCUGGGAAGACUGAACGAAAAGGGAUACGUCGAAAUCGA |
| | | CCACGCACUGCCGUUCUCGAGAACAUGGGACGACUCGUUCAACAACAAGGUCCUG |
| | | GUCCUGGGAUCGGAAAACCAGAACAAGGGAAACCAGACACCGUACGAAUACUUCA |
| | | ACGGAAAGGACAACUCGAGAGAAUGGCAGGAAUUCAAGGCAAGAGUCGAAACAUC |
| | | GAGAUUCCCGAGAUCGAAGAAGCAGAGAAUCCUGCUGCAGAAGUUCGACGAAGAC |
| | | GGAUUCAAGGAAAGAAACCUGAACGACACAAGAUACGUCAACAGAUUCCUGUGCC |
| | | AGUUCGUCGCAGACAGAAUGAGACUGACAGGAAAGGGAAAGAAGAGAGUCUUCGC |
| | | AUCGAACGGACAGAUCACAAACCUGCUGAGAGGAUUCUGGGGACUGAGAAAGGUC |
| | | AGAGCAGAAAACGACAGACACCACGCACUGGACGCAGUCGUCGUCGCAUGCUCGA |
| | | CAGUCGCAAUGCAGCAGAAGAUCACAAGAUUCGUCAGAUACAAGGAAAUGAACGC |
| | | AUUCGACGGAAAGACAAUCGACAAGGAAACAGGAGAAGUCCUGCACCAGAAGACA |
| | | CACUUCCCGCAGCCGUGGGAAUUCUUCGCACAGGAAGUCAUGAUCAGAGUCUUCG |
| | | GAAAGCCGGACGGAAAGCCGGAAUUCGAAGAAGCAGACACACUGGAAAAGCUGAG |
| | | AACACUGCUGGCAGAAAAGCUGUCGUCGAGACCGGAAGCAGUCCACGAAUACGUC |
| | | ACACCGCUGUUCGUCUCGAGAGCACCGAACAGAAAGAUGUCGGGACAGGGACACA |
| | | UGGAAACAGUCAAGUCGGCAAAGAGACUGGACGAAGGAGUCUCGGUCCUGAGAGU |
| | | CCCGCUGACACAGCUGAAGCUGAAGGACCUGGAAAAGAUGGUCAACAGAGAAAGA |
| | | GAACCGAAGCUGUACGAAGCACUGAAGGCAAGACUGGAAGCACACAAGGACGACC |
| | | CGGCAAAGGCAUUCGCAGAACCGUUCUACAAGUACGACAAGGCAGGAAACAGAAC |
| | | ACAGCAGGUCAAGGCAGUCAGAGUCGAACAGGUCCAGAAGACAGGAGUCUGGGUC |
| | | AGAAACCACAACGGAAUCGCAGACAACGCAACAAUGGUCAGAGUAGACGUCUUCG |
| | | AAAAGGGAGACAAGUACUACCUGGUCCCGAUCUACUCGUGGCAGGUCGCAAAGGG |
| | | AAUCCUGCCGGACAGAGCAGUCGUCCAGGGAAAGGACGAAGAAGACUGGCAGCUG |
| | | AUCGACGACUCGUUCAACUUCAAGUUCUCGCUGCACCCGAACGACCUGGUCGAAG |
| | | UCAUCACAAAGAAGGCAAGAAUGUUCGGAUACUUCGCAUCGUGCCACAGAGGAAC |
| | | AGGAAACAUCAACAUCAGAAUCCACGACCUGGACCACAAGAUCGGAAAGAACGGA |
| | | AUCCUGGAAGGAAUCGGAGUCAAGACAGCACUGUCGUUCCAGAAGUACCAGAUCG |
| | | ACGAACUGGGAAAGGAAAUCAGACCGUGCAGACUGAAGAAGAGACCGCCGGUCAG |
| | | AUCCGGAAAGAGAACAGCAGACGGAUCGGAAUUCGAAUCGCCGAAGAAGAAGAGA |
| | | AAGGUCGAAUGA |
| 715 | Exemplary open | augGACGGCUCCGGCGGCGGCUCCCCCAAGAAGAAGCGGAAGGUGGAGGACAAGC |
| | reading frame | GGCCCGCCGCCACCAAGAAGGCCGGCCAGGCCAAGAAGAAGAAGGGCGGCUCCGG |
| | for Nme2Cas9 | CGGCGGCGCCGCCUUCAAGCCCAACCCCAUCAACUACAUCCUGGGCCUGGACAUC |
| | with HiBiT tag | GGCAUCGCCUCCGUGGGCUGGGCCAUGGUGGAGAUCGACGAGGAGGAGAACCCCA |
| | (mRNA V ORF) | UCCGGCUGAUCGACCUGGGCGUGCGGGUGUUCGAGCGGGCCGAGGUGCCCAAGAC |
| | | CGGCGACUCCCUGGCCAUGGCCCGGCGGCUGGCCCGGUCCGUGCGGCGGCUGACC |
| | | CGGCGGCGGGCCCACCGGCUGCUGCGGGCCCGGCGGCUGCUGAAGCGGGAGGGCG |
| | | UGCUGCAGGCCGCCGACUUCGACGAGAACGGCCUGAUCAAGUCCCUGCCCAACAC |
| | | CCCCUGGCAGCUGCGGGCCGCCGCCCUGGACCGGAAGCUGACCCCCCUGGAGUGG |
| | | UCCGCCGUGCUGCUGCACCUGAUCAAGCACCGGGGCUACCUGUCCCAGCGGAAGA |
| | | ACGAGGGCGAGACCGCCGACAAGGAGCUGGGCGCCCUGCUGAAGGGCGUGGCCAA |
| | | CAACGCCCACGCCCUGCAGACCGGCGACUUCCGGACCCCCGCCGAGCUGGCCCUG |
| | | AACAAGUUCGAGAAGGAGUCCGGCCACAUCCGGAACCAGCGGGGCGACUACUCCC |
| | | ACACCUUCUCCCGGAAGGACCUGCAGGCCGAGCUGAUCCUGCUGUUCGAGAAGCA |
| | | GAAGGAGUUCGGCAACCCCCACGUGUCCGGCGGCCUGAAGGAGGGCAUCGAGACC |
| | | CUGCUGAUGACCCAGCGGCCCGCCCUGUCCGGCGACGCCGUGCAGAAGAUGCUGG |
| | | GCCACUGCACCUUCGAGCCCGCCGAGCCCAAGGCCGCCAAGAACACCUACACCGC |
| | | CGAGCGGUUCAUCUGGCUGACCAAGCUGAACAACCUGCGGAUCCUGGAGCAGGGC |
| | | UCCGAGCGGCCCCUGACCGACACCGAGCGGGCCACCCUGAUGGACGAGCCCUACC |
| | | GGAAGUCCAAGCUGACCUACGCCCAGGCCCGGAAGCUGCUGGGCCUGGAGGACAC |
| | | CGCCUUCUUCAAGGGCCUGCGGUACGGCAAGGACAACGCCGAGGCCUCCACCCUG |
| | | AUGGAGAUGAAGGCCUACCACGCCAUCUCCCGGGCCCUGGAGAAGGAGGGCCUGA |
| | | AGGACAAGAAGUCCCCCCUGAACCUGUCCUCCGAGCUGCAGGACGAGAUCGGCAC |
| | | CGCCUUCUCCCUGUUCAAGACCGACGAGGACAUCACCGGCCGGCUGAAGGACCGG |
| | | GUGCAGCCCGAGAUCCUGGAGGCCCUGCUGAAGCACAUCUCCUUCGACAAGUUCG |
| | | UGCAGAUCUCCCUGAAGGCCCUGCGGCGGAUCGUGCCCCUGAUGGAGCAGGGCAA |
| | | GCGGUACGACGAGGCCUGCGCCGAGAUCUACGGCGACCACUACGGCAAGAAGAAC |
| | | ACCGAGGAGAAGAUCUACCUGCCCCCCAUCCCCGCCGACGAGAUCCGGAACCCCG |
| | | UGGUGCUGCGGGCCCUGUCCCAGGCCCGGAAGGUGAUCAACGGCGUGGUGCGGCG |
| | | GUACGGCUCCCCCGCCCGGAUCCACAUCGAGACCGCCCGGGAGGUGGGCAAGUCC |
| | | UUCAAGGACCGGAAGGAGAUCGAGAAGCGGCAGGAGGAGAACCGGAAGGACCGGG |
| | | AGAAGGCCGCCGCCAAGUUCCGGGAGUACUUCCCCAACUUCGUGGGCGAGCCCAA |
| | | GUCCAAGGACAUCCUGAAGCUGCGGCUGUACGAGCAGCAGCACGGCAAGUGCCUG |
| | | UACUCCGGCAAGGAGAUCAACCUGGUGCGGCUGAACGAGAAGGGCUACGUGGAGA |
| | | UCGACCACGCCCUGCCCUUCUCCCGGACCUGGGACGACUCCUUCAACAACAAGGU |
| | | GCUGGUGCUGGGCUCCGAGAACCAGAACAAGGGCAACCAGACCCCCUACGAGUAC |
| | | UUCAACGGCAAGGACAACUCCCGGGAGUGGCAGGAGUUCAAGGCCCGGGUGGAGA |
| | | CCUCCCGGUUCCCCCGGUCCAAGAAGCAGCGGAUCCUGCUGCAGAAGUUCGACGA |
| | | GGACGGCUUCAAGGAGUGCAACCUGAACGACACCCGGUACGUGAACCGGUUCCUG |
| | | UGCCAGUUCGUGGCCGACCACAUCCUGCUGACCGGCAAGGGCAAGCGGCGGGUGU |
| | | UCGCCUCCAACGGCCAGAUCACCAACCUGCUGCGGGGCUUCUGGGGCCUGCGGAA |
| | | GGUGCGGGCCGAGAACGACCGGCACCACGCCCUGGACGCCGUGGUGGUGGCCUGC |
| | | UCCACCGUGGCCAUGCAGCAGAAGAUCACCCGGUUCGUGCGGUACAAGGAGAUGA |
| | | ACGCCUUCGACGGCAAGACCAUCGACAAGGAGACCGGCAAGGUGCUGCACCAGAA |
| | | GACCCACUUCCCCCAGCCCUGGGAGUUCUUCGCCCAGGAGGUGAUGAUCCGGGUG |
| | | UUCGGCAAGCCCGACGGCAAGCCCGAGUUCGAGGAGGCCGACACCCCCGAGAAGC |
| | | UGCGGACCCUGCUGGCCGAGAAGCUGUCCUCCCGGCCCGAGGCCGUGCACGAGUA |
| | | CGUGACCCCCCUGUUCGUGUCCCGGGCCCCCAACCGGAAGAUGUCCGGCGCCCAC |
| | | AAGGACACCCUGCGGUCCGCCAAGCGGUUCGUGAAGCACAACGAGAAGAUCUCCG |
| | | UGAAGCGGGUGUGGCUGACCGAGAUCAAGCUGGCCGACCUGGAGAACAUGGUGAA |
| | | CUACAAGAACGGCCGGGAGAUCGAGCUGUACGAGGCCCUGAAGGCCCGGCUGGAG |
| | | GCCUACGGCGGCAACGCCAAGCAGGCCUUCGACCCCAAGGACAACCCCUUCUACA |
| | | AGAAGGGCGGCCAGCUGGUGAAGGCCGUGCGGGUGGAGAAGACCCAGGAGUCCGG |
| | | CGUGCUGCUGAACAAGAAGAACGCCUACACCAUCGCCGACAACGGCGACAUGGUG |
| | | CGGGUGGACGUGUUCUGCAAGGUGGACAAGAAGGGCAAGAACCAGUACUUCAUCG |
| | | UGCCCAUCUACGCCUGGCAGGUGGCCGAGAACAUCCUGCCCGACAUCGACUGCAA |
| | | GGGCUACCGGAUCGACGACUCCUACACCUUCUGCUUCUCCCUGCACAAGUACGAC |
| | | CUGAUCGCCUUCCAGAAGGACGAGAAGUCCAAGGUGGAGUUCGCCUACUACAUCA |
| | | ACUGCGACUCCUCCAACGGCCGGUUCUACCUGGCCUGGCACGACAAGGGCUCCAA |
| | | GGAGCAGCAGUUCCGGAUCUCCACCCAGAACCUGGUGCUGAUCCAGAAGUACCAG |
| | | GUGAACGAGCUGGGCAAGGAGAUCCGGCCCUGCCGGCUGAAGAAGCGGCCCCCCG |
| | | UGCGGUCCGAGUCCGCCACCCCCGAGUCCGUGUCCGGCUGGCGGCUGUUCAAGAA |
| | | GAUCUCCUAG |
| 716 | Exemplary open | augGACGGCUCCGGCGGCGGCUCCCCCAAGAAGAAGCGGAAGGUGGAGGACAAGC |
| | reading frame | GGCCCGCCGCCACCAAGAAGGCCGGCCAGGCCAAGAAGAAGAAGGGCGGCUCCGG |
| | for NmelCas9 | CGGCGGCGCAGCAUUCAAGCCAAACUCAAUCAAUUACAUCCUGGGACUGGACAUC |
| | with HiBiT tag | GGCAUCGCAUCCGUCGGGUGGGCUAUGGUCGAAAUCGACGAGGAGGAGAACCCCA |
| | (mRNA X ORF) | UCCGCCUGAUCGAUCUGGGCGUGCGCGUGUUUGAGAGGGCAGAGGUGCCUAAGAC |
| | | CGGCGACAGCCUGGCCAUGGCACGGAGACUGGCACGCUCCGUGAGGCGCCUGACC |
| | | CGGAGAAGGGCCCACAGACUGCUGAGGACACGCCGGCUGCUGAAGAGGGAGGGCG |
| | | UGCUGCAGGCCGCCAACUUCGAUGAGAAUGGCCUGAUCAAGUCCCUGCCCAAUAC |
| | | CCCUUGGCAGCUGAGGGCAGCCGCCCUGGACCGCAAGCUGACACCUCUGGAGUGG |
| | | UCCGCCGUGCUGCUGCACCUGAUCAAGCACCGGGGCUACCUGUCUCAGAGAAAGA |
| | | ACGAGGGCGAGACAGCCGAUAAGGAGCUGGGCGCCCUGCUGAAGGGAGUGGCAGG |
| | | AAAUGCACACGCCCUGCAGACCGGCGACUUUCGCACACCAGCCGAGCUGGCCCUG |
| | | AACAAGUUCGAGAAGGAGAGCGGCCACAUCCGCAAUCAGCGGUCUGACUAUAGCC |
| | | ACACCUUCUCCCGGAAGGAUCUGCAGGCCGAGCUGAUCCUGCUGUUUGAGAAGCA |
| | | GAAGGAGUUCGGCAACCCACACGUGUCUGGCGGCCUGAAGGAGGGCAUCGAGACA |
| | | CUGCUGAUGACACAGCGGCCCGCCCUGAGCGGCGACGCAGUGCAGAAGAUGCUGG |
| | | GACACUGCACCUUUGAGCCAGCCGAGCCCAAGGCCGCCAAGAAUACCUACACAGC |
| | | CGAGCGGUUCAUCUGGCUGACAAAGCUGAACAAUCUGAGGAUCCUGGAGCAGGGA |
| | | AGCGAGCGCCCACUGACCGACACAGAGAGGGCCACCCUGAUGGAUGAGCCCUACC |
| | | GCAAGUCCAAGCUGACAUAUGCACAGGCAAGGAAGCUGCUGGGCCUGGAGGACAC |
| | | CGCCUUCUUUAAGGGCCUGAGAUACGGCAAGGAUAACGCCGAGGCCUCUACACUG |
| | | AUGGAGAUGAAGGCCUAUCACGCCAUCAGCAGGGCCCUGGAGAAGGAGGGCCUGA |
| | | AGGACAAGAAGUCCCCACUGAAUCUGUCUCCCGAGCUGCAGGAUGAGAUCGGCAC |
| | | CGCCUUUAGCCUGUUCAAGACCGACGAGGAUAUCACAGGCAGACUGAAGGACAGG |
| | | AUCCAGCCAGAGAUCCUGGAGGCCCUGCUGAAGCACAUCAGCUUUGAUAAGUUCG |
| | | UGCAGAUCAGCCUGAAGGCCCUGCGGAGGAUCGUGCCACUGAUGGAGCAGGGCAA |
| | | GAGGUACGACGAGGCCUGCGCCGAAAUCUACGGCGAUCACUAUGGCAAGAAGAAC |
| | | ACAGAGGAGAAAAUCUACCUGCCCCCUAUCCCCGCCGAUGAGAUCAGGAACCCUG |
| | | UGGUGCUGCGCGCCCUGUCUCAGGCAAGAAAAGUGAUCAACGGAGUGGUGCGCCG |
| | | GUACGGCAGCCCCGCCAGAAUCCACAUCGAGACAGCCAGGGAAGUGGGCAAGUCC |
| | | UUUAAGGACAGAAAGGAGAUCGAGAAGAGGCAGGAGGAGAACAGAAAGGAUAGGG |
| | | AGAAGGCCGCCGCCAAGUUCAGAGAGUACUUUCCUAAUUUCGUGGGCGAGCCAAA |
| | | GUCCAAGGAUAUCCUGAAGCUGAGGCUGUACGAGCAGCAGCACGGCAAGUGUCUG |
| | | UAUUCUGGCAAGGAGAUCAACCUGGGCCGCCUGAAUGAGAAGGGCUAUGUGGAGA |
| | | UCGACCACGCCCUGCCUUUUUCUCGGACCUGGGACGAUAGCUUCAACAAUAAGGU |
| | | GCUGGUGCUGGGCUCUGAGAACCAGAAUAAGGGCAACCAGACACCCUACGAGUAU |
| | | UUCAACGGCAAGGACAAUAGCCGCGAGUGGCAGGAGUUUAAGGCAAGGGUGGAGA |
| | | CAAGCAGGUUCCCUCGGUCCAAGAAGCAGAGAAUCCUGCUGCAGAAGUUUGACGA |
| | | GGAUGGCUUCAAGGAGAGGAACCUGAAUGACACCCGCUACGUGAAUCGGUUUCUG |
| | | UGCCAGUUCGUGGCCGAUAGAAUGAGGCUGACCGGCAAGGGCAAGAAGAGAGUGU |
| | | UUGCCUCCAACGGCCAGAUCACAAAUCUGCUGAGGGGCUUCUGGGGCCUGAGAAA |
| | | GGUGAGGGCAGAGAACGACAGGCACCACGCACUGGAUGCAGUGGUGGUGGCAUGU |
| | | UCUACCGUGGCCAUGCAGCAGAAGAUCACACGCUUUGUGCGGUAUAAGGAGAUGA |
| | | AUGCCUUCGACGGCAAGACCAUCGAUAAGGAGACAGGCGAGGUGCUGCACCAGAA |
| | | GACACACUUUCCUCAGCCAUGGGAGUUCUUUGCCCAGGAAGUGAUGAUCCGGGUG |
| | | UUUGGCAAGCCUGACGGCAAGCCAGAGUUCGAGGAGGCCGAUACCCUGGAGAAGC |
| | | UGAGAACACUGCUGGCAGAGAAGCUGAGCUCCAGGCCCGAGGCAGUGCACGAGUA |
| | | CGUGACCCCACUGUUCGUGUCUAGAGCCCCCAACAGGAAGAUGAGCGGCCAGGGC |
| | | CACAUGGAGACAGUGAAGUCCGCCAAGAGACUGGACGAGGGCGUGUCUGUGCUGA |
| | | GGGUGCCUCUGACACAGCUGAAGCUGAAGGAUCUGGAGAAGAUGGUGAAUCGCGA |
| | | GCGGGAGCCAAAGCUGUAUGAGGCCCUGAAGGCAAGGCUGGAGGCACACAAGGAC |
| | | GAUCCUGCCAAGGCCUUUGCCGAGCCAUUCUACAAGUAUGAUAAGGCCGGCAACA |
| | | GAACCCAGCAGGUGAAGGCCGUGAGGGUGGAGCAGGUGCAGAAGACAGGCGUGUG |
| | | GGUGCGCAACCACAAUGGCAUCGCCGACAAUGCUACCAUGGUGCGGGUGGACGUG |
| | | UUUGAGAAGGGCGAUAAGUACUAUCUGGUGCCCAUCUACAGCUGGCAGGUGGCCA |
| | | AGGGCAUCCUGCCUGAUAGAGCCGUGGUGCAGGGCAAGGACGAGGAGGAUUGGCA |
| | | GCUGAUCGACGAUUCCUUCAACUUUAAGUUCUCUCUGCACCCCAAUGACCUGGUG |
| | | GAAGUGAUCACCAAGAAGGCCAGGAUGUUUGGCUACUUCGCCUCCUGCCACCGCG |
| | | GCACAGGCAACAUCAAUAUCCGGAUCCACGACCUGGAUCACAAGAUCGGCAAGAA |
| | | CGGCAUCCUGGAGGGCAUCGGCGUGAAGACAGCCCUGAGCUUCCAGAAGUAUCAG |
| | | AUCGACGAGCUGGGCAAGGAGAUCAGACCUUGUAGGCUGAAGAAGCGCCCACCCG |
| | | UGCGGUCCGAGUCCGCCACCCCCGAGUCCGUGUCCGGCUGGCGGCUGUUCAAGAA |
| | | GAUCUCCUAG |
| 717 | Exemplary amino | augGACGGCUCCGGCGGCGGCUCCCCCAAGAAGAAGCGGAAGGUGGAGGACAAGC |
| | acid open | GGCCCGCCGCCACCAAGAAGGCCGGCCAGGCCAAGAAGAAGAAGGGCGGCUCCGG |
| | reading frame | CGGCGGCGCCGCCUUCAAGCCCAACUCCAUCAACUACAUCCUGGGCCUGGACAUC |
| | for Nme1Cas9 | GGCAUCGCCUCCGUGGGCUGGGCCAUGGUGGAGAUCGACGAGGAGGAGAACCCCA |
| | with HiBiT tag | UCCGGCUGAUCGACCUGGGCGUGCGGGUGUUCGAGCGGGCCGAGGUGCCCAAGAC |
| | (mRNA Y ORF) | CGGCGACUCCCUGGCCAUGGCCCGGCGGCUGGCCCGGUCCGUGCGGCGGCUGACC |
| | | CGGCGGCGGGCCCACCGGCUGCUGCGGACCCGGCGGCUGCUGAAGCGGGAGGGCG |
| | | UGCUGCAGGCCGCCAACUUCGACGAGAACGGCCUGAUCAAGUCCCUGCCCAACAC |
| | | CCCCUGGCAGCUGCGGGCCGCCGCCCUGGACCGGAAGCUGACCCCCCUGGAGUGG |
| | | UCCGCCGUGCUGCUGCACCUGAUCAAGCACCGGGGCUACCUGUCCCAGCGGAAGA |
| | | ACGAGGGCGAGACCGCCGACAAGGAGCUGGGCGCCCUGCUGAAGGGCGUGGCCGG |
| | | CAACGCCCACGCCCUGCAGACCGGCGACUUCCGGACCCCCGCCGAGCUGGCCCUG |
| | | AACAAGUUCGAGAAGGAGUCCGGCCACAUCCGGAACCAGCGGUCCGACUACUCCC |
| | | ACACCUUCUCCCGGAAGGACCUGCAGGCCGAGCUGAUCCUGCUGUUCGAGAAGCA |
| | | GAAGGAGUUCGGCAACCCCCACGUGUCCGGCGGCCUGAAGGAGGGCAUCGAGACC |
| | | CUGCUGAUGACCCAGCGGCCCGCCCUGUCCGGCGACGCCGUGCAGAAGAUGCUGG |
| | | GCCACUGCACCUUCGAGCCCGCCGAGCCCAAGGCCGCCAAGAACACCUACACCGC |
| | | CGAGCGGUUCAUCUGGCUGACCAAGCUGAACAACCUGCGGAUCCUGGAGCAGGGC |
| | | UCCGAGCGGCCCCUGACCGACACCGAGCGGGCCACCCUGAUGGACGAGCCCUACC |
| | | GGAAGUCCAAGCUGACCUACGCCCAGGCCCGGAAGCUGCUGGGCCUGGAGGACAC |
| | | CGCCUUCUUCAAGGGCCUGCGGUACGGCAAGGACAACGCCGAGGCCUCCACCCUG |
| | | AUGGAGAUGAAGGCCUACCACGCCAUCUCCCGGGCCCUGGAGAAGGAGGGCCUGA |
| | | AGGACAAGAAGUCCCCCCUGAACCUGUCCCCCGAGCUGCAGGACGAGAUCGGCAC |
| | | CGCCUUCUCCCUGUUCAAGACCGACGAGGACAUCACCGGCCGGCUGAAGGACCGG |
| | | AUCCAGCCCGAGAUCCUGGAGGCCCUGCUGAAGCACAUCUCCUUCGACAAGUUCG |
| | | UGCAGAUCUCCCUGAAGGCCCUGCGGCGGAUCGUGCCCCUGAUGGAGCAGGGCAA |
| | | GCGGUACGACGAGGCCUGCGCCGAGAUCUACGGCGACCACUACGGCAAGAAGAAC |
| | | ACCGAGGAGAAGAUCUACCUGCCCCCCAUCCCCGCCGACGAGAUCCGGAACCCCG |
| | | UGGUGCUGCGGGCCCUGUCCCAGGCCCGGAAGGUGAUCAACGGCGUGGUGCGGCG |
| | | GUACGGCUCCCCCGCCCGGAUCCACAUCGAGACCGCCCGGGAGGUGGGCAAGUCC |
| | | UUCAAGGACCGGAAGGAGAUCGAGAAGCGGCAGGAGGAGAACCGGAAGGACCGGG |
| | | AGAAGGCCGCCGCCAAGUUCCGGGAGUACUUCCCCAACUUCGUGGGCGAGCCCAA |
| | | GUCCAAGGACAUCCUGAAGCUGCGGCUGUACGAGCAGCAGCACGGCAAGUGCCUG |
| | | UACUCCGGCAAGGAGAUCAACCUGGGCCGGCUGAACGAGAAGGGCUACGUGGAGA |
| | | UCGACCACGCCCUGCCCUUCUCCCGGACCUGGGACGACUCCUUCAACAACAAGGU |
| | | GCUGGUGCUGGGCUCCGAGAACCAGAACAAGGGCAACCAGACCCCCUACGAGUAC |
| | | UUCAACGGCAAGGACAACUCCCGGGAGUGGCAGGAGUUCAAGGCCCGGGUGGAGA |
| | | CCUCCCGGUUCCCCCGGUCCAAGAAGCAGCGGAUCCUGCUGCAGAAGUUCGACGA |
| | | GGACGGCUUCAAGGAGCGGAACCUGAACGACACCCGGUACGUGAACCGGUUCCUG |
| | | UGCCAGUUCGUGGCCGACCGGAUGCGGCUGACCGGCAAGGGCAAGAAGCGGGUGU |
| | | UCGCCUCCAACGGCCAGAUCACCAACCUGCUGCGGGGCUUCUGGGGCCUGCGGAA |
| | | GGUGCGGGCCGAGAACGACCGGCACCACGCCCUGGACGCCGUGGUGGUGGCCUGC |
| | | UCCACCGUGGCCAUGCAGCAGAAGAUCACCCGGUUCGUGCGGUACAAGGAGAUGA |
| | | ACGCCUUCGACGGCAAGACCAUCGACAAGGAGACCGGCGAGGUGCUGCACCAGAA |
| | | GACCCACUUCCCCCAGCCCUGGGAGUUCUUCGCCCAGGAGGUGAUGAUCCGGGUG |
| | | UUCGGCAAGCCCGACGGCAAGCCCGAGUUCGAGGAGGCCGACACCCUGGAGAAGC |
| | | UGCGGACCCUGCUGGCCGAGAAGCUGUCCUCCCGGCCCGAGGCCGUGCACGAGUA |
| | | CGUGACCCCCCUGUUCGUGUCCCGGGCCCCCAACCGGAAGAUGUCCGGCCAGGGC |
| | | CACAUGGAGACCGUGAAGUCCGCCAAGCGGCUGGACGAGGGCGUGUCCGUGCUGC |
| | | GGGUGCCCCUGACCCAGCUGAAGCUGAAGGACCUGGAGAAGAUGGUGAACCGGGA |
| | | GCGGGAGCCCAAGCUGUACGAGGCCCUGAAGGCCCGGCUGGAGGCCCACAAGGAC |
| | | GACCCCGCCAAGGCCUUCGCCGAGCCCUUCUACAAGUACGACAAGGCCGGCAACC |
| | | GGACCCAGCAGGUGAAGGCCGUGCGGGUGGAGCAGGUGCAGAAGACCGGCGUGUG |
| | | GGUGCGGAACCACAACGGCAUCGCCGACAACGCCACCAUGGUGCGGGUGGACGUG |
| | | UUCGAGAAGGGCGACAAGUACUACCUGGUGCCCAUCUACUCCUGGCAGGUGGCCA |
| | | AGGGCAUCCUGCCCGACCGGGCCGUGGUGCAGGGCAAGGACGAGGAGGACUGGCA |
| | | GCUGAUCGACGACUCCUUCAACUUCAAGUUCUCCCUGCACCCCAACGACCUGGUG |
| | | GAGGUGAUCACCAAGAAGGCCCGGAUGUUCGGCUACUUCGCCUCCUGCCACCGGG |
| | | GCACCGGCAACAUCAACAUCCGGAUCCACGACCUGGACCACAAGAUCGGCAAGAA |
| | | CGGCAUCCUGGAGGGCAUCGGCGUGAAGACCGCCCUGUCCUUCCAGAAGUACCAG |
| | | AUCGACGAGCUGGGCAAGGAGAUCCGGCCCUGCCGGCUGAAGAAGCGGCCCCCCG |
| | | UGCGGUCCGAGUCCGCCACCCCCGAGUCCGUGUCCGGCUGGCGGCUGUUCAAGAA |
| | | GAUCUCCUAG |
| 718 | Exemplary open | augGACGGCUCCGGCGGCGGCUCCCCCAAGAAGAAGCGGAAGGUGGAGGACAAGC |
| | reading frame | GGCCCGCCGCCACCAAGAAGGCCGGCCAGGCCAAGAAGAAGAAGGGCGGCUCCGG |
| | for Nme3Cas9 | CGGCGCCGCCUUCAAGCCCAACCCCAUCAACUACAUCCUGGGCCUGGACAUCGGC |
| | with HiBiT tag | AUCGCCUCCGUGGGCUGGGCCAUGGUGGAGAUCGACGAGGAGGAGAACCCCAUCC |
| | (mRNA Z ORF) | GGCUGAUCGACCUGGGCGUGCGGGUGUUCGAGCGGGCCGAGGUGCCCAAGACCGG |
| | | CGACUCCCUGGCCAUGGCCCGGCGGCUGGCCCGGUCCGUGCGGCGGCUGACCCGG |
| | | CGGCGGGCCCACCGGCUGCUGCGGGCCCGGCGGCUGCUGAAGCGGGAGGGCGUGC |
| | | UGCAGGCCGCCGACUUCGACGAGAACGGCCUGAUCAAGUCCCUGCCCAACACCCC |
| | | CUGGCAGCUGCGGGCCGCCGCCCUGGACCGGAAGCUGACCCCCCUGGAGUGGUCC |
| | | GCCGUGCUGCUGCACCUGAUCAAGCACCGGGGCUACCUGUCCCAGCGGAAGAACG |
| | | AGGGCGAGACCGCCGACAAGGAGCUGGGCGCCCUGCUGAAGGGCGUGGCCGACAA |
| | | CGCCCACGCCCUGCAGACCGGCGACUUCCGGACCCCCGCCGAGCUGGCCCUGAAC |
| | | AAGUUCGAGAAGGAGUGCGGCCACAUCCGGAACCAGCGGGGCGACUACUCCCACA |
| | | CCUUCUCCCGGAAGGACCUGCAGGCCGAGCUGAACCUGCUGUUCGAGAAGCAGAA |
| | | GGAGUUCGGCAACCCCCACGUGUCCGGCGGCCUGAAGGAGGGCAUCGAGACCCUG |
| | | CUGAUGACCCAGCGGCCCGCCCUGUCCGGCGACGCCGUGCAGAAGAUGCUGGGCC |
| | | ACUGCACCUUCGAGCCCGCCGAGCCCAAGGCCGCCAAGAACACCUACACCGCCGA |
| | | GCGGUUCAUCUGGCUGACCAAGCUGAACAACCUGCGGAUCCUGGAGCAGGGCUCC |
| | | GAGCGGCCCCUGACCGACACCGAGCGGGCCACCCUGAUGGACGAGCCCUACCGGA |
| | | AGUCCAAGCUGACCUACGCCCAGGCCCGGAAGCUGCUGUCCCUGGAGGACACCGC |
| | | CUUCUUCAAGGGCCUGCGGUACGGCAAGGACAACGCCGAGGCCUCCACCCUGAUG |
| | | GAGAUGAAGGCCUACCACACCAUCUCCCGGGCCCUGGAGAAGGAGGGCCUGAAGG |
| | | ACAAGAAGUCCCCCCUGAACCUGUCCCCCGAGCUGCAGGACGAGAUCGGCACCGC |
| | | CUUCUCCCUGUUCAAGACCGACGAGGACAUCACCGGCCGGCUGAAGGACCGGAUC |
| | | CAGCCCGAGAUCCUGGAGGCCCUGCUGAAGCACAUCUCCUUCGACAAGUUCGUGC |
| | | AGAUCUCCCUGAAGGCCCUGCGGCGGAUCGUGCCCCUGAUGGAGCAGGGCAAGCG |
| | | GUACGACGAGGCCUGCGCCGAGAUCUACGGCGACCACUACGGCAAGAAGAACACC |
| | | GAGGAGAAGAUCUACCUGCCCCCCAUCCCCGCCGACGAGAUCCGGAACCCCGUGG |
| | | UGCUGCGGGCCCUGUCCCAGGCCCGGAAGGUGAUCAACGGCGUGGUGCGGCGGUA |
| | | CGGCUCCCCCGCCCGGAUCCACAUCGAGACCGCCCGGGAGGUGGGCAAGUCCUUC |
| | | AAGGACCGGAAGGAGAUCGAGAAGCGGCAGGAGGAGAACCGGAAGGACCGGGAGA |
| | | AGGCCGCCGCCAAGUUCCGGGAGUACUUCCCCAACUUCGUGGGCGAGCCCAAGUC |
| | | CAAGGACAUCCUGAAGCUGCGGCUGUACGAGCAGCAGCACGGCAAGUGCCUGUAC |
| | | UCCGGCAAGGAGAUCAACCUGGGCCGGCUGAACGAGAAGGGCUACGUGGAGAUCG |
| | | ACCACGCCCUGCCCUUCUCCCGGACCUGGGACGACUCCUUCAACAACAAGGUGCU |
| | | GGUGCUGGGCUCCGAGAACCAGAACAAGGGCAACCAGACCCCCUACGAGUACUUC |
| | | AACGGCAAGGACAACUCCCGGGAGUGGCAGGAGUUCAAGGCCCGGGUGGAGACCU |
| | | CCCGGUUCCCCCGGUCCAAGAAGCAGCGGAUCCUGCUGCAGAAGUUCGACGAGGA |
| | | CGGCUUCAAGGAGCGGAACCUGAACGACACCCGGUACGUGAACCGGUUCCUGUGC |
| | | CAGUUCGUGGCCGACCGGAUGCGGCUGACCGGCAAGGGCAAGAAGCGGGUGUUCG |
| | | CCUCCAACGGCCAGAUCACCAACCUGCUGCGGGGCUUCUGGGGCCUGCGGAAGGU |
| | | GCGGGCCGAGAACGACCGGCACCACGCCCUGGACGCCGUGGUGGUGGCCUGCUCC |
| | | ACCGUGGCCAUGCAGCAGAAGAUCACCCGGUUCGUGCGGUACAAGGAGAUGAACG |
| | | CCUUCGACGGCAAGACCAUCGACAAGGAGACCGGCGAGGUGCUGCACCAGAAGAC |
| | | CCACUUCCCCCAGCCCUGGGAGUUCUUCGCCCAGGAGGUGAUGAUCCGGGUGUUC |
| | | GGCAAGCCCGACGGCAAGCCCGAGUUCGAGGAGGCCGACACCCCCGAGAAGCUGC |
| | | GGACCCUGCUGGCCGAGAAGCUGUCCUCCCGGCCCGAGGCCGUGCACGAGUACGU |
| | | GACCCCCCUGUUCGUGUCCCGGGCCCCCAACCGGAAGAUGUCCGGCCAGGGCCAC |
| | | AUGGAGACCGUGAAGUCCGCCAAGCGGCUGGACGAGGGCGUGUCCGUGCUGCGGG |
| | | UGCCCCUGACCCAGCUGAAGCUGAAGGACCUGGAGAAGAUGGUGAACCGGGAGCG |
| | | GGAGCCCAAGCUGUACGAGGCCCUGAAGGCCCGGCUGGAGGCCCACAAGGACGAC |
| | | CCCGCCAAGGCCUUCGCCGAGCCCUUCUACAAGUACGACAAGGCCGGCAACCGGA |
| | | CCCAGCAGGUGAAGGCCGUGCGGGUGGAGCAGGUGCAGAAGACCGGCGUGUGGGU |
| | | GCGGAACCACAACGGCAUCGCCGACAACGCCACCAUGGUGCGGGUGGACGUGUUC |
| | | GAGAAGGGCGACAAGUACUACCUGGUGCCCAUCUACUCCUGGCAGGUGGCCAAGG |
| | | GCAUCCUGCCCGACCGGGCCGUGGUGGCCUACGCCGACGAGGAGGACUGGACCGU |
| | | GAUCGACGAGUCCUUCCGGUUCAAGUUCGUGCUGUACUCCAACGACCUGAUCAAG |
| | | GUGCAGCUGAAGAAGGACUCCUUCCUGGGCUACUUCUCCGGCCUGGACCGGGCCA |
| | | CCGGCGCCAUCUCCCUGCGGGAGCACGACCUGGAGAAGUCCAAGGGCAAGGACGG |
| | | CAUGCACCGGAUCGGCGUGAAGACCGCCCUGUCCUUCCAGAAGUACCAGAUCGAC |
| | | GAGAUGGGCAAGGAGAUCCGGCCCUGCCGGCUGAAGAAGCGGCCCCCCGUGCGGU |
| | | CCGAGUCCGCCACCCCCGAGUCCGUGUCCGGCUGGCGGCUGUUCAAGAAGAUCUC |
| | | CUAG |

TABLE 4B
Additional Sequences

| | | |
|---|---|---|
| G000502 | 758 | ACACAAAUACCAGUCCAGCGGUUUUAGAGCUAGAAAUAGCAAGUUAAAAUAAGGCUAGUCCGUUAUCAACUUGAAAA |
| (unmodified) | | AGUGGCACCGAGUCGGUGCUUUU |
| G000502 | 759 | mA*mC*mA*CAAAUACCAGUCCAGCGGUUUUAGAmGmCmUmAmGmAmAmAmUmAmGmCAAGUUAAAAUAAGGCUAGU |
| | | CCGUUAUCAmAmCmUmUmGmAmAmAmAmAmGmUmGmGmCmAmCmCmGmAmGmUmCmGmGmUmGmCmU*mU*mU*mU |
| RNAWT- | 760 | GGCGCTTCGCCAGCCATTCCTGCGTTGTAGCTCCCTTTCTCATTTCGGAAACGAAATGAGAACCGTTGCTACAATAA |
| 145 | | GGCCGTCTGAAAAGATGTGCCGCAACGCTCTGCCCCTTAAAGCTTCTGCTTTAAGGGGCATCGTTTAT |
| RNA9- | 761 | GGCGCTTCGCCAGCCATTCCTGCGTTGTAGCCTTCTGAAAAGAAGGCTACAATAAGGCCGTCTGAAAAGATGTGCCG |
| 102 | | CAACGCTCTGCTTCTGCATCGTtt |
| RNA7- | 762 | GGCGCTTCGCCAGCCATTCCTGCGTTGTAGCTCCCTCTGAAAAGCCGTTGCTACAATAAGGCCGTCTGAAAAGATGT |
| 106 | | GCCGCAACGCTCTGCTTCTGCATCGTtt |
| RNA8- | 763 | GGCGCTTCGCCAGCCATTCCTGCGTTGTAGCTCCCTTTCTGAAAAGAACCGTTGCTACAATAAGGCCGTCTGAAAAG |
| 106 | | ATGTGCCGCAACGCTCTGCTTCTGCAtt |
| RNA6- | 764 | GGCGCTTCGCCAGCCATTCCTGCGTTGTAGCTCCCTTTCTGAAAAGAACCGTTGCTACAATAAGGCCGTCTGAAAAG |
| 110 | | ATGTGCCGCAACGCTCTGCTTCTGCATCGTtt |
| RNA6b- | 765 | GGCGCTTCGCCAGCCATTCCTGCGTTGTAGCCTTCTCATTGAAAAATGAGAAGGCTACAATAAGGCCGTCTGAAAAG |
| 110 | | ATGTGCCGCAACGCTCTGCTTCTGCATCGttt |
| RNA5- | 766 | GGCGCTTCGCCAGCCATTCCTGCGTTGTAGCTCCCTCTGAAAAGCCGTTGCTACAATAAGGCCGTCTGAAAAGATGT |
| 112 | | GCCGCAACGCTCTGCCCCTTTTCTAAGGGGCAtt |
| RNA4- | 767 | GGCGCTTCGCCAGCCATTCCTGCGTTGTAGCTCCCTTTCTGAAAAGAACCGTTGCTACAATAAGGCCGTCTGAAAAG |
| 116 | | ATGTGCCGCAACGCTCTGCCCCTTTTCTAAGGGGCAtt |
| RNA3- | 768 | gGGGCGCTTCGCCAGCCATTCCTGCGTTGTAGCTCCCTTTCTCAGGAAACTGAGAACCGTTGCTACAATAAGGCCGT |
| 122 | | CTGAAAAGATGTGCCGCAACGCTCTGCCCCTTTTCTAAGGGGCAT |
| RNA2- | 769 | GGCGCTTCGCCAGCCATTCCTGCGTTGTAGCTCCCTTCTCAGGAAACTGAGAACCGTTGCTACAATAAGGCCGTCTG |
| 126 | | AAAAGATGTGCCGCAACGCTCTGCCCCTtTTCTAAGGGGCATCGtttT |
| RNA17- | 770 | GGCGCTTCGCCAGCCATTCCTGCGttGtAGCtCCCtGAAACCGttGCtACAAtAAGGCCGtGAAAAtGtGCCGCAAC |
| 101 | | GCtCtGCCttCtGGCAtCGtttT |
| RNA18- | 771 | GGCGCTTCGCCAGCCATTCCTGCGttGtAGCtCCCtGAAACCGttGCtACAAtAAGGCCGtCGAAAGAtGtGCCGCA |
| 103 | | ACGCtCtGCCttCtGGCAtCGtttT |
| RNA15- | 772 | GGCGCTTCGCCAGCCATTCCTGCGttGtAGCtCCCtGAAACCGttGCtACAAtAAGGCCGtGAAAAtGtGCCGCAAC |
| 105 | | GCtCtGCCGCttCtGCGGCAtCGtttT |
| RNA16- | 773 | GGCGCTTCGCCAGCCATTCCTGCGttGtAGCtCCCtGAAACCGttGCtACAAtAAGGCCGtCtGAAAAGAtGtGCCG |
| 105 | | CAACGCtCtGCCttCtGGCAtCGtttT |
| RNA13- | 774 | GGCGCTTCGCCAGCCATTCCTGCGttGtAGCtCCCttCGGAAACGACCGttGCtACAAtAAGGCCGtGAAAAtGtGC |
| 107 | | CGCAACGCtCtGCCttCtGGCAtCGtttT |
| RNA14- | 775 | GGCGCTTCGCCAGCCATTCCTGCGttGtAGCtCCCtGAAACCGttGCtACAAtAAGGCCGtCtGAAAAGAtGtGCCG |
| 109 | | CAACGCtCtGCCGCttCtGCGGCAtCGtttT |
| RNA12- | 776 | GGCGCTTCGCCAGCCATTCCTGCGttGtAGCtCCCttCGGAAACGACCGttGCtACAAtAAGGCCGtCtGAAAAGAt |
| 111 | | GtGCCGCAACGCtCtGCCttCtGGCAtCGtttT |
| RNA11- | 777 | GGCGCTTCGCCAGCCATTCCTGCGttGtAGCtCCCttCGGAAACGACCGttGCtACAAtAAGGCCGtGAAAAtGtGC |
| 113 | | CGCAACGCtCtGCCGCttCtGCGGCAtCGtttTTT |
| RNA10- | 778 | GGCGCTTCGCCAGCCATTCCTGCGttGtAGCtCCCttCGGAAACGACCGttGCtACAAtAAGGCCGtCtGAAAAGAt |
| 115 | | GtGCCGCAACGCtCtGCCGCttCtGCGGCAtCGtttT |
| R10B- | 779 | GGCGCTTCGCCAGCCATTCCTGCGTTGTAGCTCCCTTGGAAACACCGTTGCTACAATAAGGCCGTTGAAAAATGTGC |
| 111 | | CGCAACGCTCTGCCCTTCTGGGCATCGTTTTTT |
| R10E- | 780 | GGCGCTTCGCCAGCCATTCCTGCGTTGTAGCTCCCTTGGAAACACCGTTGCTACAATAAGGCCGTCGAAAGATGTGC |
| 109 | | CGCAACGCTCTGCCCTTCTGGGCATCGTTTT |
| R10F- | 781 | GGCGCTTCGCCAGCCATTCCTGCGTTGTAGCTCCCTTGGAAACACCGTTGCTACAATAAGGCCGTCTGAAAAGATGT |
| 113 | | GCCGCAACGCTCTGCCGCTTCTGCGGCATCGTTTT |
| R10G- | 782 | GGCGCTTCGCCAGCCATTCCTGCGTTGTAGCTCCCTTCGGAAACGACCGTTGCTACAATAAGGCCGTCTGAAAAGAT |
| 113 | | GTGCCGCAACGCTCTGCCGTTCTCGGCATCGTTTT |
| R10H- | 783 | GGCGCTTCGCCAGCCATTCCTGCGTTGTAGCTCCCTTCGGAAACGACCGTTGCTACAATAAGGCCGTCTGAAAAGAT |
| 113 | | GTGCCGCAACGCTCTGCCCTTCTGGGCATCGTTTT |
| R10I- | 784 | GGCGCTTCGCCAGCCATTCCTGCGTTGTAGCTCCCTTCGGAAACGACCGTTGCTACAATAAGGCCGTTGAAAAATGT |
| 113 | | GCCGCAACGCTCTGCCGCTTCTGCGGCATCGTTTT |
| R10J- | 785 | GGCGCTTCGCCAGCCATTCCTGCGTTGTAGCTCCCTTCGGAAACGACCGTTGCTACAATAAGGCCGTCGAAAGATGT |
| 113 | | GCCGCAACGCTCTGCCGCTTCTGCGGCATCGTTTT |
| R19- | 786 | GGCGCTTCGCCAGCCATTCCTGCGTTGTAGCTCCCTTCGGAACGACCGTTGCTACAATAAGGCCGTCTGAAAGATGT |
| 112 | | GCCGCAACGCTCTGCCGCTCTGCGGCATCGTTTT |
| ES99 | 787 | GGCGCTTCGCCAGCCATTCCTGCGTTGTAGCTCCCGAAACGTTGCTACAATAAGGCCGTCTGAAAAGATGTGCCGCA |
| | | ACGCTCTGCTTCTGCATCGTT |
| ES121 | 788 | gGGGCGCTTCGCCAGCCATTCCTGCGTTGTAGCTCCCGAAACGTTGCTACAATAAGGCCCGTCTGAAAAGATGTGCC |
| | | GCAACGCTCTGCCCCTTAAAGCTTCTGCTTTAAGGGGCATCGTTTA |
| ES111 | 789 | GGCGCTTCGCCAGCCATTCCTGCGTTGTAGCTCCCGAAACGTTGCTACAATAAGGCCGTCTGAAAAGATGTGCCGCA |
| | | ACGCTCTGCCCCTTTTCTAAGGGGCATCGTTTA |
| ES105 | 790 | GGCGCTTCGCCAGCCATTCCTGCGTTGTAGCTCCCGAAACGTTGCTACAATAAGGCCGTCTGAAAAGATGTGCCGCA |
| | | ACGCTCTGCttCTGCATCGTTTAtttT |
| | 791- | Not used |
| | 799 | |
| G02306 | 800 | mC*mC*mA*mAmGUGmUmCUmUCmCAGUAmCGAUmUUGmGUUGmUmAmGmCUCCCmU(L1)mCmCGUUmGmCUAmCA |
| 6 | | AU*AAGmGmCCmGmUmC(L1)mGmAmUGUGCmCGmCAAmCGCUCUmGmCC(L1)GGCAUCG*mU*mU |
| G02306 | 801 | mC*mU*mU*mCmACCmAmGGmAGmAAGCCmGUCAmCACmGUUGmUmAmGmCUCCCmU(L1)mCmCGUUmGmCUAmCA |
| 7 | | AU*AAGmGmCCmGmUmC(L1)mGmAmUGUGCmCGmCAAmCGCUCUmGmCC(L1)GGCAUCG*mU*mU |
| G02306 | 802 | mC*mU*mU*mCmACCmAmAGmAGmAAGCCmGUCAmCACmGUUGmUmAmGmCUCCCmU(L1)mCmCGUUmGmCUAmCA |
| 8 | | AU*AAGmGmCCmGmUmC(L1)mGmAmUGUGCmCGmCAAmCGCUCUmGmCC(L1)GGCAUCG*mU*mU |
| G02306 | 803 | CUUCACCAGGAGAAGCCGUCACACmGUUGmUmAmGmCUCCCmU(L1)mCmCGUUmGmCUAmCAAU*AAGmGmCCmGm |
| 9 | | UmC(L1)mGmAmUGUGCmCGmCAAmCGCUCUmGmCC(L1)GGCAUCG*mU*mU |
| G02307 | 804 | mC*mU*mU*CACCAGGAGAAGCCGUCACACmGUUGmUmAmGmCUCCCmU(L1)mCmCGUUmGmCUAmCAAU*AAGmG |
| 0 | | mCCmGmUmC(L1)mGmAmUGUGCmCGmCAAmCGCUCUmGmCC(L1)GGCAUCG*mU*mU |
| G02307 | 805 | mC*mU*mU*mCACCAGGAGAAGCCGUCACACmGUUGmUmAmGmCUCCCmU(L1)mCmCGUUmGmCUAmCAAU*AAGm |
| 1 | | GmCCmGmUmC(L1)mGmAmUGUGCmCGmCAAmCGCUCUmGmCC(L1)GGCAUCG*mU*mU |
| G02307 | 806 | mC*mU*mU*CmACCAGGAGAAGCCGUCACACmGUUGmUmAmGmCUCCCmU(L1)mCmCGUUmGmCUAmCAAU*AAGm |
| 2 | | GmCCmGmUmC(L1)mGmAmUGUGCmCGmCAAmCGCUCUmGmCC(L1)GGCAUCG*mU*mU |
| G02307 | 807 | mC*mU*mU*CACCAmGGAGAAGCCGUCACACmGUUGmUmAmGmCUCCCmU(L1)mCmCGUUmGmCUAmCAAU*AAGm |
| 3 | | GmCCmGmUmC(L1)mGmAmUGUGCmCGmCAAmCGCUCUmGmCC(L1)GGCAUCG*mU*mU |
| G02307 | 808 | mC*mU*mU*CACCAGGmAGAAGCCGUCACACmGUUGmUmAmGmCUCCCmU(L1)mCmCGUUmGmCUAmCAAU*AAGm |
| 4 | | GmCCmGmUmC(L1)mGmAmUGUGCmCGmCAAmCGCUCUmGmCC(L1)GGCAUCG*mU*mU |
| G02307 | 809 | mC*mU*mU*CACCAGGAGmAAGCCGUCACACmGUUGmUmAmGmCUCCCmU(L1)mCmCGUUmGmCUAmCAAU*AAGm |
| 5 | | GmCCmGmUmC(L1)mGmAmUGUGCmCGmCAAmCGCUCUmGmCC(L1)GGCAUCG*mU*mU |
| G02307 | 810 | mC*mU*mU*CACCAGGAGAAGCCmGUCACACmGUUGmUmAmGmCUCCCmU(L1)mCmCGUUmGmCUAmCAAU*AAGm |
| 6 | | GmCCmGmUmC(L1)mGmAmUGUGCmCGmCAAmCGCUCUmGmCC(L1)GGCAUCG*mU*mU |
| G02307 | 811 | mC*mU*mU*CACCAGGAGAAGCCGUCAmCACmGUUGmUmAmGmCUCCCmU(L1)mCmCGUUmGmCUAmCAAU*AAGm |
| 7 | | GmCCmGmUmC(L1)mGmAmUGUGCmCGmCAAmCGCUCUmGmCC(L1)GGCAUCG*mU*mU |
| G02307 | 812 | mC*mU*mU*CmACCmAmGGmAGmAAGCCmGUCAmCACmGUUGmUmAmGmCUCCCmU(L1)mCmCGUUmGmCUAmCAA |
| 8 | | U*AAGmGmCCmGmUmC(L1)mGmAmUGUGCmCGmCAAmCGCUCUmGmCC(L1)GGCAUCG*mU*mU |
| G02307 | 813 | mC*mU*mU*mCACCmAmGGmAGmAAGCCmGUCAmCACmGUUGmUmAmGmCUCCCmU(L1)mCmCGUUmGmCUAmCAA |
| 9 | | U*AAGmGmCCmGmUmC(L1)mGmAmUGUGCmCGmCAAmCGCUCUmGmCC(L1)GGCAUCG*mU*mU |
| G02308 | 814 | mC*mU*mU*mCmACCAmGGmAGmAAGCCmGUCAmCACmGUUGmUmAmGmCUCCCmU(L1)mCmCGUUmGmCUAmCAA |
| 0 | | U*AAGmGmCCmGmUmC(L1)mGmAmUGUGCmCGmCAAmCGCUCUmGmCC(L1)GGCAUCG*mU*mU |
| G02308 | 815 | mC*mU*mU*mCmACCmAGGmAGmAAGCCmGUCAmCACmGUUGmUmAmGmCUCCCmU(L1)mCmCGUUmGmCUAmCAA |
| 1 | | U*AAGmGmCCmGmUmC(L1)mGmAmUGUGCmCGmCAAmCGCUCUmGmCC(L1)GGCAUCG*mU*mU |
| G02308 | 816 | mC*mU*mU*mCmACCmAmGGAGmAAGCCmGUCAmCACmGUUGmUmAmGmCUCCCmU(L1)mCmCGUUmGmCUAmCAA |
| 2 | | U*AAGmGmCCmGmUmC(L1)mGmAmUGUGCmCGmCAAmCGCUCUmGmCC(L1)GGCAUCG*mU*mU |
| G02308 | 817 | mC*mU*mU*mCmACCmAmGGmAGAAGCCmGUCAmCACmGUUGmUmAmGmCUCCCmU(L1)mCmCGUUmGmCUAmCAA |
| 3 | | U*AAGmGmCCmGmUmC(L1)mGmAmUGUGCmCGmCAAmCGCUCUmGmCC(L1)GGCAUCG*mU*mU |
| G02308 | 818 | mC*mU*mU*mCmACCmAmGGmAGmAAGCCGUCAmCACmGUUGmUmAmGmCUCCCmU(L1)mCmCGUUmGmCUAmCAA |
| 4 | | U*AAGmGmCCmGmUmC(L1)mGmAmUGUGCmCGmCAAmCGCUCUmGmCC(L1)GGCAUCG*mU*mU |
| G02308 | 819 | mC*mU*mU*mCmACCmAmGGmAGmAAGCCmGUCACACmGUUGmUmAmGmCUCCCmU(L1)mCmCGUUmGmCUAmCAA |
| 5 | | U*AAGmGmCCmGmUmC(L1)mGmAmUGUGCmCGmCAAmCGCUCUmGmCC(L1)GGCAUCG*mU*mU |
| G02308 | 820 | CUUCACCAAGAGAAGCCGUCACACmGUUGmUmAmGmCUCCCmU(L1)mCmCGUUmGmCUAmCAAU*AAGmGmCCmGm |
| 6 | | UmC(L1)mGmAmUGUGCmCGmCAAmCGCUCUmGmCC(L1)GGCAUCG*mU*mU |
| G02308 | 821 | mC*mU*mU*CACCAAGAGAAGCCGUCACACmGUUGmUmAmGmCUCCCmU(L1)mCmCGUUmGmCUAmCAAU*AAGmG |
| 7 | | mCCmGmUmC(L1)mGmAmUGUGCmCGmCAAmCGCUCUmGmCC(L1)GGCAUCG*mU*mU |
| G02308 | 822 | mC*mU*mU*mCACCAAGAGAAGCCGUCACACmGUUGmUmAmGmCUCCCmU(L1)mCmCGUUmGmCUAmCAAU*AAGm |
| 8 | | GmCCmGmUmC(L1)mGmAmUGUGCmCGmCAAmCGCUCUmGmCC(L1)GGCAUCG*mU*mU |
| G02308 | 823 | mC*mU*mU*CmACCAAGAGAAGCCGUCACACmGUUGmUmAmGmCUCCCmU(L1)mCmCGUUmGmCUAmCAAU*AAGm |
| 9 | | GmCCmGmUmC(L1)mGmAmUGUGCmCGmCAAmCGCUCUmGmCC(L1)GGCAUCG*mU*mU |
| G02309 | 824 | mC*mU*mU*CACCAmAGAGAAGCCGUCACACmGUUGmUmAmGmCUCCCmU(L1)mCmCGUUmGmCUAmCAAU*AAGm |
| 0 | | GmCCmGmUmC(L1)mGmAmUGUGCmCGmCAAmCGCUCUmGmCC(L1)GGCAUCG*mU*mU |
| G02309 | 825 | mC*mU*mU*CACCAAGmAGAAGCCGUCACACmGUUGmUmAmGmCUCCCmU(L1)mCmCGUUmGmCUAmCAAU*AAGm |
| 1 | | GmCCmGmUmC(L1)mGmAmUGUGCmCGmCAAmCGCUCUmGmCC(L1)GGCAUCG*mU*mU |
| G02309 | 826 | mC*mU*mU*CACCAAGAGmAAGCCGUCACACmGUUGmUmAmGmCUCCCmU(L1)mCmCGUUmGmCUAmCAAU*AAGm |
| 2 | | GmCCmGmUmC(L1)mGmAmUGUGCmCGmCAAmCGCUCUmGmCC(L1)GGCAUCG*mU*mU |
| G02309 | 827 | mC*mU*mU*CACCAAGAGAAGCCmGUCACACmGUUGmUmAmGmCUCCCmU(L1)mCmCGUUmGmCUAmCAAU*AAGm |
| 3 | | GmCCmGmUmC(L1)mGmAmUGUGCmCGmCAAmCGCUCUmGmCC(L1)GGCAUCG*mU*mU |
| G02309 | 828 | mC*mU*mU*CACCAAGAGAAGCCGUCAmCACmGUUGmUmAmGmCUCCCmU(L1)mCmCGUUmGmCUAmCAAU*AAGm |
| 4 | | GmCCmGmUmC(L1)mGmAmUGUGCmCGmCAAmCGCUCUmGmCC(L1)GGCAUCG*mU*mU |
| G02309 | 829 | mC*mU*mU*CmACCmAmAGmAGmAAGCCmGUCAmCACmGUUGmUmAmGmCUCCCmU(L1)mCmCGUUmGmCUAmCAA |
| 5 | | U*AAGmGmCCmGmUmC(L1)mGmAmUGUGCmCGmCAAmCGCUCUmGmCC(L1)GGCAUCG*mU*mU |
| G02309 | 830 | mC*mU*mU*mCACCmAmAGmAGmAAGCCmGUCAmCACmGUUGmUmAmGmCUCCCmU(L1)mCmCGUUmGmCUAmCA |
| 6 | | AU*AAGmGmCCmGmUmC(L1)mGmAmUGUGCmCGmCAAmCGCUCUmGmCC(L1)GGCAUCG*mU*mU |
| G02309 | 831 | mC*mU*mU*mCmACCAmAGmAGmAAGCCmGUCAmCACmGUUGmUmAmGmCUCCCmU(L1)mCmCGUUmGmCUAmCAA |
| 7 | | U*AAGmGmCCmGmUmC(L1)mGmAmUGUGCmCGmCAAmCGCUCUmGmCC(L1)GGCAUCG*mU*mU |
| G02309 | 832 | mC*mU*mU*mCmACCmAAGmAGmAAGCCmGUCAmCACmGUUGmUmAmGmCUCCCmU(L1)mCmCGUUmGmCUAmCAA |
| 8 | | U*AAGmGmCCmGmUmC(L1)mGmAmUGUGCmCGmCAAmCGCUCUmGmCC(L1)GGCAUCG*mU*mU |
| G02309 | 833 | mC*mU*mU*mCmACCmAmAGAGmAAGCCmGUCAmCACmGUUGmUmAmGmCUCCCmU(L1)mCmCGUUmGmCUAmCAA |
| 9 | | U*AAGmGmCCmGmUmC(L1)mGmAmUGUGCmCGmCAAmCGCUCUmGmCC(L1)GGCAUCG*mU*mU |
| G02310 | 834 | mC*mU*mU*mCmACCmAmAGmAGAAGCCmGUCAmCACmGUUGmUmAmGmCUCCCmU(L1)mCmCGUUmGmCUAmCA |
| 0 | | AU*AAGmGmCCmGmUmC(L1)mGmAmUGUGCmCGmCAAmCGCUCUmGmCC(L1)GGCAUCG*mU*mU |
| G02310 | 835 | mC*mU*mU*mCmACCmAmAGmAGmAAGCCGUCAmCACmGUUGmUmAmGmCUCCCmU(L1)mCmCGUUmGmCUAmCAA |
| 1 | | U*AAGmGmCCmGmUmC(L1)mGmAmUGUGCmCGmCAAmCGCUCUmGmCC(L1)GGCAUCG*mU*mU |
| G02310 | 836 | mC*mU*mU*mCmACCmAmAGmAGmAAGCCmGUCACACmGUUGmUmAmGmCUCCCmU(L1)mCmCGUUmGmCUAmCAA |
| 2 | | U*AAGmGmCCmGmUmC(L1)mGmAmUGUGCmCGmCAAmCGCUCUmGmCC(L1)GGCAUCG*mU*mU |
| G02310 | 837 | CCAAGUGUCUUCCAGUACGAUUUGmGUUGmUmAmGmCUCCCmU(L1)mCmCGUUmGmCUAmCAAU*AAGmGmCCmGm |
| 3 | | UmC(L1)mGmAmUGUGCmCGmCAAmCGCUCUmGmCC(L1)GGCAUCG*mU*mU |
| G02310 | 838 | mC*mC*mA*AGUGUCUUCCAGUACGAUUUGmGUUGmUmAmGmCUCCCmU(L1)mCmCGUUmGmCUAmCAAU*AAGmG |
| 4 | | mCCmGmUmC(L1)mGmAmUGUGCmCGmCAAmCGCUCUmGmCC(L1)GGCAUCG*mU*mU |
| G02310 | 839 | mC*mC*mA*mAGUGUCUUCCAGUACGAUUUGmGUUGmUmAmGmCUCCCmU(L1)mCmCGUUmGmCUAmCAAU*AAGm |
| 5 | | GmCCmGmUmC(L1)mGmAmUGUGCmCGmCAAmCGCUCUmGmCC(L1)GGCAUCG*mU*mU |
| G02310 | 840 | mC*mC*mA*AmGUGUCUUCCAGUACGAUUUGmGUUGmUmAmGmCUCCCmU(L1)mCmCGUUmGmCUAmCAAU*AAGm |
| 6 | | GmCCmGmUmC(L1)mGmAmUGUGCmCGmCAAmCGCUCUmGmCC(L1)GGCAUCG*mU*mU |
| G02310 | 841 | mC*mC*mA*AGUGUmCUUCCAGUACGAUUUGmGUUGmUmAmGmCUCCCmU(L1)mCmCGUUmGmCUAmCAAU*AAGm |
| 7 | | GmCCmGmUmC(L1)mGmAmUGUGCmCGmCAAmCGCUCUmGmCC(L1)GGCAUCG*mU*mU |
| G02310 | 842 | mC*mC*mA*AGUGUCUmUCCAGUACGAUUUGmGUUGmUmAmGmCUCCCmU(L1)mCmCGUUmGmCUAmCAAU*AAGm |
| 8 | | GmCCmGmUmC(L1)mGmAmUGUGCmCGmCAAmCGCUCUmGmCC(L1)GGCAUCG*mU*mU |
| G02310 | 843 | mC*mC*mA*AGUGUCUUCmCAGUACGAUUUGmGUUGmUmAmGmCUCCCmU(L1)mCmCGUUmGmCUAmCAAU*AAGm |
| 9 | | GmCCmGmUmC(L1)mGmAmUGUGCmCGmCAAmCGCUCUmGmCC(L1)GGCAUCG*mU*mU |
| G02311 | 844 | mC*mC*mA*AGUGUCUUCCAGUAmCGAUUUGmGUUGmUmAmGmCUCCCmU(L1)mCmCGUUmGmCUAmCAAU*AAGm |
| 0 | | GmCCmGmUmC(L1)mGmAmUGUGCmCGmCAAmCGCUCUmGmCC(L1)GGCAUCG*mU*mU |
| G02311 | 845 | mC*mC*mA*AGUGUCUUCCAGUACGAUmUUGmGUUGmUmAmGmCUCCCmU(L1)mCmCGUUmGmCUAmCAAU*AAGm |
| 1 | | GmCCmGmUmC(L1)mGmAmUGUGCmCGmCAAmCGCUCUmGmCC(L1)GGCAUCG*mU*mU |
| G02311 | 846 | mC*mC*mA*AmGUGmUmCUmUCmCAGUAmCGAUmUUGmGUUGmUmAmGmCUCCCmU(L1)mCmCGUUmGmCUAmCAA |
| 2 | | U*AAGmGmCCmGmUmC(L1)mGmAmUGUGCmCGmCAAmCGCUCUmGmCC(L1)GGCAUCG*mU*mU |
| G02311 | 847 | mC*mC*mA*mAGUGmUmCUmUCmCAGUAmCGAUmUUGmGUUGmUmAmGmCUCCCmU(L1)mCmCGUUmGmCUAmCAA |
| 3 | | U*AAGmGmCCmGmUmC(L1)mGmAmUGUGCmCGmCAAmCGCUCUmGmCC(L1)GGCAUCG*mU*mU |
| G02311 | 848 | mC*mC*mA*mAmGUGUmCUmUCmCAGUAmCGAUmUUGmGUUGmUmAmGmCUCCCmU(L1)mCmCGUUmGmCUAmCAA |
| 4 | | U*AAGmGmCCmGmUmC(L1)mGmAmUGUGCmCGmCAAmCGCUCUmGmCC(L1)GGCAUCG*mU*mU |
| G02311 | 849 | mC*mC*mA*mAmGUGmUCUmUCmCAGUAmCGAUmUUGmGUUGmUmAmGmCUCCCmU(L1)mCmCGUUmGmCUAmCAA |
| 5 | | U*AAGmGmCCmGmUmC(L1)mGmAmUGUGCmCGmCAAmCGCUCUmGmCC(L1)GGCAUCG*mU*mU |
| G02311 | 850 | mC*mC*mA*mAmGUGmUmCUUCmCAGUAmCGAUmUUGmGUUGmUmAmGmCUCCCmU(L1)mCmCGUUmGmCUAmCAA |
| 6 | | U*AAGmGmCCmGmUmC(L1)mGmAmUGUGCmCGmCAAmCGCUCUmGmCC(L1)GGCAUCG*mU*mU |
| G02311 | 851 | mC*mC*mA*mAmGUGmUmCUmUCCAGUAmCGAUmUUGmGUUGmUmAmGmCUCCCmU(L1)mCmCGUUmGmCUAmCAA |
| 7 | | U*AAGmGmCCmGmUmC(L1)mGmAmUGUGCmCGmCAAmCGCUCUmGmCC(L1)GGCAUCG*mU*mU |
| G02311 | 852 | mC*mC*mA*mAmGUGmUmCUmUCmCAGUACGAUmUUGmGUUGmUmAmGmCUCCCmU(L1)mCmCGUUmGmCUAmCAA |
| 8 | | U*AAGmGmCCmGmUmC(L1)mGmAmUGUGCmCGmCAAmCGCUCUmGmCC(L1)GGCAUCG*mU*mU |
| G02311 | 853 | mC*mC*mA*mAmGUGmUmCUmUCmCAGUAmCGAUUUGmGUUGmUmAmGmCUCCCmU(L1)mCmCGUUmGmCUAmCAA |
| 9 | | U*AAGmGmCCmGmUmC(L1)mGmAmUGUGCmCGmCAAmCGCUCUmGmCC(L1)GGCAUCG*mU*mU |
| G02312 | 854 | mC*mU*mU*CACCmAGGAGAAGCCGUCACACmGUUGmUmAmGmCUCCCmU(L1)mCmCGUUmGmCUAmCAAU*AAGm |
| 0 | | GmCCmGmUmC(L1)mGmAmUGUGCmCGmCAAmCGCUCUmGmCC(L1)GGCAUCG*mU*mU |
| G02312 | 855 | mC*mU*mU*CACCmAAGAGAAGCCGUCACACmGUUGmUmAmGmCUCCCmU(L1)mCmCGUUmGmCUAmCAAU*AAGm |
| 1 | | GmCCmGmUmC(L1)mGmAmUGUGCmCGmCAAmCGCUCUmGmCC(L1)GGCAUCG*mU*mU |
| G02312 | 856 | mC*mC*mA*AGUGmUCUUCCAGUACGAUUUGmGUUGmUmAmGmCUCCCmU(L1)mCmCGUUmGmCUAmCAAU*AAGm |
| 2 | | GmCCmGmUmC(L1)mGmAmUGUGCmCGmCAAmCGCUCUmGmCC(L1)GGCAUCG*mU*mU |
| TR0182 | 857 | mC*mG*mA*UGAGAACCGUUGCUACAAUAAGGCCGUCUGAAAAGAUGUGCCGCAACGCUCUGCCCUUAAGCUUCUGC |
| 27 | | UUAAGGGCAUCGUUU*mA*mU*mU |
| TR0182 | 858 | mC*mG*mA*UGAGAACCGUUGCUACAAUAAGGCCGUCGAAAGAUGUGCCGCAACGCUCUGCCCUUAAGCUUCUGCUU |
| 28 | | AAGGGCAUCGUUU*mA*mU*mU |
| TR0182 | 859 | mC*mG*mA*UGAGAACCGUUGCUACAAUAAGGCCGUGAAAAUGUGCCGCAACGCUCUGCCCUUAAGCUUCUGCUUAA |
| 29 | | GGGCAUCGUUU*mA*mU*mU |
| TR0182 | 860 | mC*mG*mA*UGAGAACCGUUGCUACAAUAAGGCCGGAAAUGUGCCGCAACGCUCUGCCCUUAAGCUUCUGCUUAAGG |
| 30 | | GCAUCGUUU*mA*mU*mU |
| TR0182 | 861 | mC*mG*mA*UGAGAACCGUUGCUACAAUAAGGCCGAAAGUGCCGCAACGCUCUGCCCUUAAGCUUCUGCUUAAGGGC |
| 31 | | AUCGUUU*mA*mU*mU |
| TR0182 | 862 | mC*mG*mA*UGAGAACCGUUGCUACAAUAAGGCCGUCUGAAAAGAUGUGCCGCAACGCUCUGCCCUUAAGUUCUCUU |
| 32 | | AAGGGCAUCGUUU*mA*mU*mU |
| TR0182 | 863 | mC*mG*mA*UGAGAACCGUUGCUACAAUAAGGCCGUCUGAAAAGAUGUGCCGCAACGCUCUGCCCUUAAUUCUUUAA |
| 33 | | GGGCAUCGUUU*mA*mU*mU |
| TR0182 | 864 | mC*mG*mA*UGAGAACCGUUGCUACAAUAAGGCCGUCUGAAAAGAUGUGCCGCAACGCUCUGCCCUUAUUCUUAAGG |
| 34 | | GCAUCGUUU*mA*mU*mU |
| TR0182 | 865 | mC*mG*mA*UGAGAACCGUUGCUACAAUAAGGCCGUCUGAAAAGAUGUGCCGCAACGCUCUGCCCUUUUCUAAGGGC |
| 35 | | AUCGUUU*mA*mU*mU |
| TR0182 | 866 | mC*mG*mA*UGAGAACCGUUGCUACAAUAAGGCCGUCUGAAAAGAUGUGCCGCAACGCUCUGCCCUUUCUAGGGCAU |
| 36 | | CGUUU*mA*mU*mU |
| TR0182 | 867 | mC*mG*mA*UGAGAACCGUUGCUACAAUAAGGCCGUCUGAAAAGAUGUGCCGCAACGCUCUGCCCUUCUGGGCAUCG |
| 37 | | UUU*mA*mU*mU |
| TR0182 | 868 | mC*mG*mA*UGAGAACCGUUGCUACAAUAAGGCCGUCUGAAAAGAUGUGCCGCAACGCUCUGCCUUCUGGCAUCGUU |
| 38 | | U*mA*mU*mU |
| TR0182 | 869 | mC*mG*mA*UGAGAACCGUUGCUACAAUAAGGCCGUCUGAAAAGAUGUGCCGCAACGCUCUGCUUCUGCAUCGUUU* |
| 39 | | mA*mU*mU |
| TR0182 | 870 | mC*mG*mA*UGAGAACCGUUGCUACAAUAAGGCCGUCUGAAAAGAUGUGCCGCAACGCUCUGUUCUCAUCGUUU*mA |
| 40 | | *mU*mU |
| TR0182 | 871 | mC*mG*mA*UGAGAACCGUUGCUACAAUAAGGCCGUGAAAAUGUGCCGCAACGCUCUGCCUUCUGGCAUCGUUU*mA |
| 41 | | *mU*mU |
| TR0182 | 872 | mC*mG*mA*UGAGAACCGUUGCUACAAUAAGGCCGUCUGAAAAGAUGUGCCGCAACGCUCUGCCCUUAAGCUUCUGC |
| 42 | | UUAAGGGCAUCG*mU*mU*mU |
| CR0186 | 873 | mA*mC*mU*mCmACUmCmAmCmCCmACmACmAmGACAmCAmCmGUUmGmUmAmGmCmUCCCmU*mU*mU*mC*mU*m |
| 48 | | C*mA*mU*mC*mG |
| CR0186 | 874 | mG*mU*mG*mUmGUCmCmCmUmCUmCCmCCmAmCCCGmUCmCmGUUmGmUmAmGmCmUCCCmU*mU*mU*mC*U*mC |
| 56 | | *mA*mU*mC*mG |
| CR0184 | 875 | mG*mU*mG*UGUCCCUCUCCCCACCCGUCCGUUGUAGCUCCCUUUCUCA*mU*mC*mG |
| 73 | | |
| CR0184 | 876 | mG*mU*mG*UGUCCCUCCACCCGUCCmGUUGmUmAmGmCmUCCCmUmUmUmCmUmCmA*mU*mC*mG |
| 74 | | |
| CR0184 | 877 | mG*mU*mG*mUmGmUCCCmUmCmUmCmCCCmAmCCCGmUCCGUUGUAGCUCCCUUUCUCA*mU*mC*mG |
| 75 | | |
| CR0184 | 878 | mG*mU*mG*mUmGmUCCCmUmCmUmCmCCCmAmCCCGmUCCmGUUGmUmAmGmCmUCCCmUmUmUmCmUmCmA*mU* |
| 76 | | mC*mG |
| TR0184 | 879 | mC*mG*mA*UGAGAACCGUUGCUACAAUAAGGCCGUCUmGmAmAmAAGAUGUGCCGCAACGCUCUGCCCUUAAGCmU |
| 77 | | mUmCmUGCUUAAGGGCAUCGUU*mU*mA*mU |
| TR0184 | 880 | mC*mG*mA*UGAGAACCGUUGCUACAAUAAGGCCGUCUmGmAmAmAAGAUGUGCCGCAACGCUCUGCCCUUAAGCmG |
| 78 | | mAmAmAGCUUAAGGGCAUCGUU*mU*mA*mU |
| TR0184 | 881 | mCmGmAUGAGAACCGUUGCUACAAUAAGGCCGUCUmGmAmAmAAGAUGUGCCGCAACGCUCUGCCCUUAAGCmUmUm |
| 79 | | CmUGCUUAAGGGCAUCGUUmUmAmU |
| TR0184 | 882 | mC*mG*mA*UGAGAAAGGUUGCUACAAUAAGGCCGUCUmGmAmAmAAGAUGUGCCGCAACGCUCUGCCCUUAAGCmU |
| 80 | | mUmCmUGCUUAAGGGCAUCGUU*mU*mA*mU |
| TR0184 | 883 | mC*mG*mA*UGAGAACCGUUGCUACAAUAAGGCCGUCUmGmAmAmAAGAUGUGCCGCAACGCUCUGCCCUUAAGCmU |
| 81 | | mUmCmUGCUUAAGGGCAUCGU*U*mU*mU |
| TR0184 | 884 | mC*mG*mA*UGAGAACCGUUGCUACAAUAAGGCCGUCUmGmAmAmAAGAUGUGCCGCAACGCUCUGCCCUUAAGCmU |
| 82 | | mUmCmUGCUUAAGGGCAUCGU*mU*mU*mU |
| TR0184 | 885 | mC*mG*mA*UGAGAACCGUUGCUACAAUAAGGCCGUCUmGmAmAmAAGAUGUGCCGCAACGCUCUGCCCUUAAGCmU |
| 83 | | mUmCmUGCUUAAGGGCAUCG*mU*mU*mU |
| TR0184 | 886 | mC*mG*mA*mUmGmAmGmAmAmCmCGUUGmCUACAAmUAAGmGmCmCmGmUmCmUmGmAmAmAmAmGmAmUGUmGmC |
| 84 | | mCGCAmAmCmGmCmUmCmUmGmCmCCmUmUmAmAmGmCmUmUmCmUmGmCmUmUmAmAGmGmGCAUCGmUmU*mU*m |
| | | A*mU |
| TR0184 | 887 | mC*mG*mA*mUmGmAmGmAmAmAmGGUUGmCUACAAmUAAGmGmCmCmGmUmCmUmGmAmAmAmAmGmAmUGUmGmC |
| 85 | | mCGCAmAmCmGmCmUmCmUmGmCmCCmUmUmAmAmGmCmUmUmCmUmGmCmUmUmAmAGmGmGCAUCGmUmU*mU*m |
| | | A*mU |
| TR0184 | 888 | mC*mG*mA*mUmGmAmGmAmAmCmCGUUGmCUACAAmUAAGmGmCmCmGmUmCmUmGmAmAmAmAmGmAmUGUmGmC |
| 86 | | mCGCAmAmCmGmCmUmCmUmGmCmCCmUmUmAmAmGmCmUmUmCmUmGmCmUmUmAmAGmGmGCAUCG*mU*mU*mU |
| TR0184 | 889 | mC*mG*mA*mUmGmAmGmAmAmCmCGUUGmCUACAAmUAAGmGmCmCmGmUmCmGmAmAmAmGmAmUGUmGmCmCGC |
| 87 | | AmAmCmGmCmUmCmUmGmCmCCmUmUmCmUmGmGCAUCGmUmU*mU*mA*mU |
| TR0184 | 890 | mC*mG*mA*mUmGmAmGmAmAmCmCGUUGmCUACAAmUAAGmGmCmCmGmUmCmGmAmAmAmGmAmUGUmGmCmCGC |
| 88 | | AmAmCmGmCmUmCmUmGmCmCCmUmUmCmUmGmGCAUCG*mU*mU*mU |
| TR0184 | 891 | mC*mG*mA*mUmGmAmGmAmAmCmCGUUGmCUACAAmUAAGGCCGUCUmGmAmAmAAGAUGUGCCGCAACGCUCUGC |
| 89 | | CCUUAAGCmUmUmCmUGCUUAAGGGCAUCGUU*mU*mA*mU |
| TR0184 | 892 | mC*mG*mA*UGAGAACCGUUGCUACAAUAAGmGmCmCmGmUmCmUmGmAmAmAmAmGmAmUGUmGmCmCGCAACGCU |
| 90 | | CUGCCCUUAAGCmUmUmCmUGCUUAAGGGCAUCGUU*mU*mA*mU |
| TR0184 | 893 | mC*mG*mA*UGAGAACCGUUGCUACAAUAAGGCCGUCUmGmAmAmAAGAUGUGCCGCAmAmCmGmCmUmCmUmGmCm |
| 91 | | CCmUmUmAmAmGmCmUmUmCmUmGmCmUmUmAmAGmGmGCAUCGmUmU*mU*mA*mU |
| TR0184 | 894 | mC*mG*mA*mUmGmAmGmAmAmCmCGUUGmCUACAAmUAAGmGmCmCmGmUmCmUmGmAmAmAmAmGmAmUGUmGmC |
| 92 | | mCGCAACGCUCUGCCCUUAAGCmUmUmCmUGCUUAAGGGCAUCGUU*mU*mA*mU |
| TR0184 | 895 | mC*mG*mA*mUmGmAmGmAmAmCmCGUUGmCUACAAmUAAGGCCGUCUmGmAmAmAAGAUGUGCCGCAmAmCmGmCm |
| 93 | | UmCmUmGmCmCCmUmUmAmAmGmCmUmUmCmUmGmCmUmUmAmAGmGmGCAUCGmUmU*mU*mA*mU |
| TR0184 | 896 | mC*mG*mA*UGAGAACCGUUGCUACAAUAAGmGmCmCmGmUmCmUmGmAmAmAmAmGmAmUGUmGmCmCGCAmAmCm |
| 94 | | GmCmUmCmUmGmCmCCmUmUmAmAmGmCmUmUmCmUmGmCmUmUmAmAGmGmGCAUCGmUmU*mU*mA*mU |
| TR0184 | 897 | mC*mG*mA*mUmGmAmGmAmAmCmCGUUmGmCUACAmAmUAAGGCCGUCUmGmAmAmAAGAUGUGCCGCAACGCUCU |
| 95 | | GCCCUUAAGCmUmUmCmUGCUUAAGGGCAUCGUU*mU*mA*mU |
| TR0184 | 898 | mC*mG*mA*UGAGAACCGUUGCUACAAUAmAGmGmCmCmGmUmCmUmGmAmAmAmAmGmAmUGmUmGmCmCmGmCAA |
| 96 | | CGCUCUGCCCUUAAGCmUmUmCmUGCUUAAGGGCAUCGUU*mU*mA*mU |
| TR0184 | 899 | mC*mG*mA*UGAGAACCGUUGCUACAAUAAGGCCGUCUmGmAmAmAAGAUGUGCCGCmAmAmCmGmCmUmCmUmGmC |
| 97 | | mCmCmUmUmAmAmGmCmUmUmCmUmGmCmUmUmAmAGmGmGCmAmUmCmGmUmU*mU*mA*mU |
| TR0184 | 900 | mC*mG*mA*mUmGmAmGmAmAmCmCGUUmGmCUACAmAmUAmAGmGmCmCmGmUmCmUmGmAmAmAmAmGmAmUGmU |
| 98 | | mGmCmCGmCAACGCUCUGCCCUUAAGCmUmUmCmUGCUUAAGGGCAUCGUU*mU*mA*mU |
| TR0184 | 901 | mC*mG*mA*mUmGmAmGmAmAmCmCGUUmGmCUACAmAmUAAGGCCGUCUmGmAmAmAAGAUGUGCCGCmAmAmCmG |
| 99 | | mCmUmCmUmGmCmCmCmUmUmAmAmGmCmUmUmCmUmGmCmUmUmAmAGmGmGCmAmUmCmGmUmU*mU*mA*mU |
| TR0185 | 902 | mC*mG*mA*UGAGAACCGUUGCUACAAUAmAGmGmCmCmGmUmCmUmGmAmAmAmAmGmAmUGmUmGmCmCGmCmAm |
| 00 | | AmCmGmCmUmCmUmGmCmCmCmUmUmAmAmGmCmUmUmCmUmGmCmUmUmAmAGmGmGCmAmUmCmGmUmU*mU*mA |
| | | *mU |
| G02749 | 903 | mC*mC*mA*mAmGUGmUmCUmUCmCAGUAmCGAUmUUGmGUUGmUmAmGmCUCCCmUmUmCmGmAmAmAmGmAmCmC |
| 2 | | GUUmGmCUAmCAAU*AAGmGmCCmGmUmCmGmAmAmAmGmAmUGUGCmCGmCAAmCGCUCUmGmCCmUmUmCmUGGC |
| | | AUCG*mU*mU |
| G02749 | 904 | mC*mC*mA*mAmGUGmUmCUmUCmCAGUAmCGAUmUUGmGUUGmUmAmGmCUCCCmUmUmCmGmAmAmAmGmAmCmC |
| 3 | | GUUmGmCUAmCAAU*AAGmGmCCmGmUmGmAmAmAmAmUGUGCmCGmCAAmCGCUCUmGmCCmUmUmCmUGGCAUCG |
| | | *mU*mU |
| G02749 | 905 | mC*mC*mA*mAmGUGmUmCUmUCmCAGUAmCGAUmUUGmGUUGmUmAmGmCUCCCmUmUmCmGmAmAmAmGmAmCmC |
| 4 | | GUUmGmCUAmCAAU*AAGmGmCCmGmGmAmAmAmUGUGCmCGmCAAmCGCUCUmGmCCmUmUmCmUGGCAUCG*mU* |
| | | mU |
| G02749 | 906 | mC*mC*mA*mAmGUGmUmCUmUCmCAGUAmCGAUmUUGmGUUGmUmAmGmCUCCCmUmUmCmGmAmAmAmGmAmCmC |
| 5 | | GUUmGmCUAmCAAU*AAGmGmCCmGmAmAmAGUGCmCGmCAAmCGCUCUmGmCCmUmUmCmUGGCAUCG*mU*mU |
| G02749 | 907 | mC*mC*mA*mAmGUGmUmCUmUCmCAGUAmCGAUmUUGmGUUGmUmAmGmCUCCCmUmGmAmAmAmCmCGUUmGmCU |
| 6 | | AmCAAU*AAGmGmCCmGmAmAmAGUGCmCGmCAAmCGCUCUmGmCCmUmUmCmUGGCAUCG*mU*mU |
| G02410 | 908 | mC*mA*mA*mGmUCUmGmUCmUGmCCUAUmUCACmCGAmGUUGmUmAmGmCUCCCmU(L1)mCmCGUUmGmCUAmCA |
| 3 | | AU*AAGmGmCCmGmUmC(L1)mGmAmUGUGCmCGmCAAmCGCUCUmGmCC(L1)GGCAUCG*mU*mU |
| G02410 | 909 | mG*mG*mU*mGmAAUmAmGGmCAmGACAGmACUUmGUCmGUUGmUmAmGmCUCCCmU(L1)mCmCGUUmGmCUAmCA |
| 4 | | AU*AAGmGmCCmGmUmC(L1)mGmAmUGUGCmCGmCAAmCGCUCUmGmCC(L1)GGCAUCG*mU*mU |
| G02410 | 910 | mG*mA*mU*mUmAAAmCmCCmGGmCCACUmUUCAmGGAmGUUGmUmAmGmCUCCCmU(L1)mCmCGUUmGmCUAmCA |
| 8 | | AU*AAGmGmCCmGmUmC(L1)mGmAmUGUGCmCGmCAAmCGCUCUmGmCC(L1)GGCAUCG*mU*mU |
| G02410 | 911 | mC*mA*mG*mUmGACmAmAGmUCmUGUCUmGCCUmAUUmGUUGmUmAmGmCUCCCmU(L1)mCmCGUUmGmCUAmCA |
| 9 | | AU*AAGmGmCCmGmUmC(L1)mGmAmUGUGCmCGmCAAmCGCUCUmGmCC(L1)GGCAUCG*mU*mU |
| G02411 | 912 | mG*mU*mU*mGmAAGmGmCGmUUmUGCACmAUGCmAAAmGUUGmUmAmGmCUCCCmU(L1)mCmCGUUmGmCUAmCA |
| 0 | | AU*AAGmGmCCmGmUmC(L1)mGmAmUGUGCmCGmCAAmCGCUCUmGmCC(L1)GGCAUCG*mU*mU |
| G02411 | 913 | mU*mC*mC*mUmGUGmAmUGmUCmAAGCUmGGUCmGAGmGUUGmUmAmGmCUCCCmU(L1)mCmCGUUmGmCUAmCA |
| 1 | | AU*AAGmGmCCmGmUmC(L1)mGmAmUGUGCmCGmCAAmCGCUCUmGmCC(L1)GGCAUCG*mU*mU |
| G02411 | 914 | mC*mA*mG*mGmUUUmUmGAmAAmGUUUAmGGUUmCGUmGUUGmUmAmGmCUCCCmU(L1)mCmCGUUmGmCUAmCA |
| 2 | | AU*AAGmGmCCmGmUmC(L1)mGmAmUGUGCmCGmCAAmCGCUCUmGmCC(L1)GGCAUCG*mU*mU |
| G02411 | 915 | mA*mU*mC*mAmGAAmUmCCmUUmACUUUmGUGAmCACmGUUGmUmAmGmCUCCCmU(L1)mCmCGUUmGmCUAmCA |
| 3 | | AU*AAGmGmCCmGmUmC(L1)mGmAmUGUGCmCGmCAAmCGCUCUmGmCC(L1)GGCAUCG*mU*mU |
| G02411 | 916 | mG*mA*mA*mGmUCCmAmUAmGAmCCUCAmUGUCmUAGmGUUGmUmAmGmCUCCCmU(L1)mCmCGUUmGmCUAmCA |
| 4 | | AU*AAGmGmCCmGmUmC(L1)mGmAmUGUGCmCGmCAAmCGCUCUmGmCC(L1)GGCAUCG*mU*mU |
| G02884 | 917 | mU*mG*mU*mCmUGCmCmUAmUUmCACCGmAUUUmUGAmGUUGmUmAmGmCUCCCmUmGmAmAmAmCmCGUUmGmCU |
| 4 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02884 | 918 | mU*mG*mC*mCmUAUmUmCAmCCmGAUUUmUGAUmUCUmGUUGmUmAmGmCUCCCmUmGmAmAmAmCmCGUUmGmCU |
| 5 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02884 | 919 | mC*mG*mA*mUmUUUmGmAUmUCmUCAAAmCAAAmUGUmGUUGmUmAmGmCUCCCmUmGmAmAmAmCmCGUUmGmCU |
| 6 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02884 | 920 | mG*mU*mA*mAmGGAmUmUCmUGmAUGUGmUAUAmUCAmGUUGmUmAmGmCUCCCmUmGmAmAmAmCmCGUUmGmCU |
| 7 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02884 | 921 | mA*mG*mA*mGmCAAmCmAGmUGmCUGUGmGCCUmGGAmGUUGmUmAmGmCUCCCmUmGmAmAmAmCmCGUUmGmCU |
| 8 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02884 | 922 | mU*mG*mG*mAmGCAmAmCAmAAmUCUGAmCUUUmGCAmGUUGmUmAmGmCUCCCmUmGmAmAmAmCmCGUUmGmCU |
| 9 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02885 | 923 | mA*mU*mG*mCmUGUmUmGUmUGmAAGGCmGUUUmGCAmGUUGmUmAmGmCUCCCmUmGmAmAmAmCmCGUUmGmCU |
| 0 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02885 | 924 | mU*mU*mU*mUmGAAmAmGUmUUmAGGUUmCGUAmUCUmGUUGmUmAmGmCUCCCmUmGmAmAmAmCmCGUUmGmCU |
| 1 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02885 | 925 | mU*mU*mA*mCmUUUmGmUGmACmACAUUmUGUUmUGAmGUUGmUmAmGmCUCCCmUmGmAmAmAmCmCGUUmGmCU |
| 2 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02885 | 926 | mG*mA*mU*mUmAAAmCmCCmGGmCCACUmUUCAmGGAmGUUGmUmAmGmCUCCCmUmGmAmAmAmCmCGUUmGmCU |
| 3 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02146 | 927 | mA*mU*mA*mUmCCAmGmAAmCCmCUGACmCCUGmCCGmGUUGmUmAmGmCUCCCmUmGmAmAmAmCmCGUUmGmCU |
| 9 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02473 | 928 | mA*mG*mG*mAmCCAmGmCCmUCmAGACAmCAAAmUACmGUUGmUmAmGmCUCCCmUmGmAmAmAmCmCGUUmGmCU |
| 9 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02474 | 929 | mC*mU*mG*mCmCUCmGmGAmCGmGCAUCmUAGAmACUmGUUGmUmAmGmCUCCCmUmGmAmAmAmCmCGUUmGmCU |
| 1 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02474 | 930 | mA*mG*mG*mCmAGAmGmGAmGGmAGCAGmACGAmUGAmGUUGmUmAmGmCUCCCmUmGmAmAmAmCmCGUUmGmCU |
| 3 | | AmCAAU*AAGmGmCCmGmUmCmGmAmAmAmGmAmUGUGCmCGmCAAmCGCUCUmGmCCmUmUmCmUGGCAUCG*mU* |
| | | mU |
| G02937 | 931 | mC*mC*mA*mAmGUGmUmCUmUCmCAGUAmCGAUmUUGmGUUGmUmAmGmCUCCCmUmGmAmAmAmCmCGUUmGmCU |
| 7 | | AmCAAUAAGmGmCCmGmUmCmGmAmAmAmGmAmUGUGCmCGmCAAmCGCUCUmGmCCmUmUmCmUGGCAUCG*mU*m |
| | | U |
| G02937 | 932 | mC*mC*mA*mAmGUGmUmCUmUCmCAGUAmCGAUmUUGmGUUGmUmAmGmCUCCCmUmGmAmAmAmCmCGUUmGmCU |
| 8 | | AmCmAmAUAAGmGmCCmGmUmCmGmAmAmAmGmAmUGUGCmCGmCAAmCGCUCUmGmCCmUmUmCmUGGCAUCG*mU |
| | | *mU |
| G02937 | 933 | mC*mC*mA*mAmGUGmUmCUmUCmCAGUAmCGAUmUUGmGUUGmUmAmGmCUCCCmUmGmAmAmAmCmCGUUmGmCU |
| 9 | | AmCAmAUAAGmGmCCmGmUmCmGmAmAmAmGmAmUGUGCmCGmCAAmCGCUCUmGmCCmUmUmCmUGGCAUCG*mU* |
| | | mU |
| G02938 | 934 | mC*mC*mA*mAmGUGmUmCUmUCmCAGUAmCGAUmUUGmGUUGmUmAmGmCUCCCmUmGmAmAmAmCmCGUUmGmCU |
| 0 | | AmCmAmAU*AAGmGmCCmGmUmCmGmAmAmAmGmAmUGUGCmCGmCAAmCGCUCUmGmCCmUmUmCmUGGCAUCG*m |
| | | U*mU |
| G02938 | 935 | mC*mC*mA*mAmGUGmUmCUmUCmCAGUAmCGAUmUUGmGUUGmUmAmGmCUCCCmUmGmAmAmAmCmCGUUmGmCU |
| 1 | | AmCAAdTAAGmGmCCmGmUmCmGmAmAmAmGmAmUGUGCmCGmCAAmCGCUCUmGmCCmUmUmCmUGGCAUCG*mU* |
| | | mU |
| G02938 | 936 | mC*mC*mA*mAmGUGmUmCUmUCmCAGUAmCGAUmUUGmGUUGmUmAmGmCUCCCmUmGmAmAmAmCmCGUUmGmCU |
| 2 | | AmCAAU*AAGmGmCCmGmUmCmGmAmAmAmGmAmUGUGCmCGmCAAmCmGCUCUmGmCCmUmUmCmUGGCAUCG*mU |
| | | *mU |
| G02938 | 937 | mC*mC*mA*mAmGUGmUmCUmUCmCAGUAmCGAUmUUGmGUUGmUmAmGmCUCCCmUmGmAmAmAmCmCGUUmGmCU |
| 3 | | AmCAAU*AAGmGmCCmGmUmCmGmAmAmAmGmAmUGUGCmCGmCAAmCGCUCmUmGmCCmUmUmCmUGGCAUCG*mU |
| | | *mU |
| G02938 | 938 | mC*mC*mA*mAmGUGmUmCUmUCmCAGUAmCGAUmUUGmGUUGmUmAmGmCUCCCmUmGmAmAmAmCmCGUUmGmCU |
| 4 | | AmCAAU*AAGmGmCCmGmUmCmGmAmAmAmGmAmUGUGCmCGmCAAmCGmCmUmCmUmGmCCmUmUmCmUGGCAUCG |
| | | *mU*mU |
| G02938 | 939 | mC*mC*mA*mAmGUGmUmCUmUCmCAGUAmCGAUmUUGmGUUGmUmAmGmCUCCCmUmGmAmAmAmCmCGUUmGmCU |
| 5 | | AmCAAU*AAGmGmCCmGmUmCmGmAmAmAmGmAmUGUGCmCGmCAAmCGmCmUmCUmGmCCmUmUmCmUGGCAUCG* |
| | | mU*mU |
| G02938 | 940 | mC*mC*mA*mAmGUGmUmCUmUCmCAGUAmCGAUmUUGmGUUGmUmAmGmCUCCCmUmGmAmAmAmCmCGUUmGmCU |
| 6 | | AmCAAU*AAGmGmCCmGmUmCmGmAmAmAmGmAmUGUGCmCGmCAAmCmGmCmUmCmUmGmCCmUmUmCmUGGCAUC |
| | | G*mU*mU |
| G02938 | 941 | mC*mC*mA*mAmGUGmUmCUmUCmCAGUAmCGAUmUUGmGUUGmUmAmGmCUCCCmUmGmAmAmAmCmCGUUmGmCU |
| 7 | | AmCAAU*AAGmGmCCmGmUmCmGmAmAmAmGmAmUGUGCmCGmCmAmAmCmGmCmUmCmUmGmCmCmUmUmCmUGGC |
| | | AUCG*mU*mU |
| G02938 | 942 | mC*mC*mA*mAmGUGmUmCUmUCmCAGUAmCGAUmUUGmGUUGmUmAmGmCUCCCmUmGmAmAmAmCmCGUUmGmCU |
| 8 | | AmCAAU*AAGmGmCCmGmUmCmGmAmAmAmGmAmUGUGCmCGmCAAmCGCUCUmGmCCmUmUmCmUGGCAUmCmG*m |
| | | U*mU |
| G02938 | 943 | mC*mC*mA*mAmGUGmUmCUmUCmCAGUAmCGAUmUUGmGUUGmUmAmGmCUCCCmUmGmAmAmAmCmCGUUmGmCU |
| 9 | | AmCAAU*AAGmGmCCmGmUmCmGmAmAmAmGmAmUGUGCmCGmCAAmCGCUCUmGmCCmUmUmCmUGGCmAmUmCmG |
| | | *mU*mU |
| G02939 | 944 | mC*mC*mA*mAmGUGmUmCUmUCmCAGUAmCGAUmUUGmGUUGmUmAmGmCUCCCmUmGmAmAmAmCmCGUUmGmCU |
| 0 | | AmCAAU*AAGmGmCCmGmUmCmGmAmAmAmGmAmUGUGCmCGmCmAmAmCmGmCmUmCmUmGmCmCmUmUmCmUGGC |
| | | mAmUmCmG*mU*mU |
| G02939 | 945 | mC*mC*mA*mAmGUGmUmCUmUCmCAGUAmCGAUmUUGmGUUGmUmAmGmCUCCCmUmGmAmAmAmCmCGUUmGmCU |
| 1 | | AmCAAU*AAGmGmCCmGmUmCmGmAmAmAmGmAmUGUGCmCGmCmAmAmCmGmCmUmCmUmGmCmCmUmUmCmUGGC |
| | | AUmCmG*mU*mU |
| G02939 | 946 | mC*mC*mA*mAmGUGmUmCUmUCmCAGUAmCGAUmUUGmGUUGmUmAmGmCUCCCmUmGmAmAmAmCmCGUUmGmCU |
| 2 | | AmCAAU*AAGmGmCCmGmUmCmGmAmAmAmGmAmUGUGCmCGmCAAmCmGmCmUmCmUmGmCCmUmUmCmUGGCAUm |
| | | CmG*mU*mU |
| G02473 | 947 | AGGACCAGCCUCAGACACAAAUACGUUGUAGCUCCCUGAAACCGUUGCUACAAUAAGGCCGUCGAAAGAUGUGCCGC |
| 9 | | AACGCUCUGCCUUCUGGCAUCGUU |
| G02474 | 948 | CUGCCUCGGACGGCAUCUAGAACUGUUGUAGCUCCCUGAAACCGUUGCUACAAUAAGGCCGUCGAAAGAUGUGCCGC |
| 1 | | AACGCUCUGCCUUCUGGCAUCGUU |
| G02474 | 949 | AGGCAGAGGAGGAGCAGACGAUGAGUUGUAGCUCCCUGAAACCGUUGCUACAAUAAGGCCGUCGAAAGAUGUGCCGC |
| 3 | | AACGCUCUGCCUUCUGGCAUCGUU |

It is understood that if a DNA sequence (comprising Ts) is referenced herein with respect to an RNA, then Ts should be replaced with Us (which may be modified or unmodified depending on the context), and vice versa.

Nucleotide modifications are indicated in Table 4 as follows: m: 2′-OMe; *: PS linkage; f: 2′-fluoro; (invd): inverted abasic; moe: 2′-moe; e: ENA; d: deoxyribonucleotide (also note that T is always a deoxyribonucleotide); x: UNA. In the sgRNA modified sequences, in certain embodiments, each A, C, G, U, and N is independently a ribose sugar (2′-OH). In certain embodiments, each A, C, G, U, and N is a ribose sugar (2′-OH). Thus, for example, mA represents 2′-O-methyl adenosine; xA represents a UNA nucleotide with an adenine nucleobase; eA represents an ENA nucleotide with an adenine nucleobase; and dA represents an adenosine deoxyribonucleotide. As used herein, (L1) refers to an internal linker having a bridging length of about 15-21 atoms.

sgRNA designations are sometimes provided with one or more leading zeroes immediately following the G. This does not affect the meaning of the designation. Thus, for example, G000282, G0282, G00282, and G282 refer to the same sgRNA. Similarly, crRNA and or trRNA designations are sometimes provided with one or more leading zeroes immediately following the CR or TR, respectively, which does not affect the meaning of the designation. Thus, for example, CR000100, CR00100, CR0100, and CR100 refer to the same crRNA, and TR000200, TR00200, TR0200, and TR200 refer to the same trRNA.

### EXAMPLES

The following examples are provided to illustrate certain disclosed embodiments and are not to be construed as limiting the scope of this disclosure in any way.

### Example 1. Materials and Methods

In Vitro Transcription (“IVT”) of Nuclease mRNA

Capped and polyadenylated mRNA containing N1-methyl pseudo-U was generated by in vitro transcription using routine methods. For example, a plasmid DNA containing a T7 promoter, a sequence for transcription, and a polyadenylation region was linearized with XbaI per manufacturer's protocol. The XbaI was inactivated by heating. The linearized plasmid was purified from enzyme and buffer salts. The IVT reaction to generate modified mRNA was performed by incubating at 37° C.: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase; 1 U/μL Murine RNase inhibitor (NEB); 0.004 U/μL Inorganic E. coli pyrophosphatase (NEB); and 1× reaction buffer. TURBO DNase (Thermo Fisher) was added to a final concentration of 0.01 U/μL, and the reaction was incubated at 37° C. to remove the DNA template.

The mRNA was purified using a MegaClear Transcription Clean-up kit (Thermo Fisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA was purified using LiCl precipitation, ammonium acetate precipitation, and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanalyzer (Agilent).

Streptococcus pyogenes (“Spy”) Cas9 mRNA was generated from plasmid DNA encoding an open reading frame according to SEQ ID Nos: 661-665 (see sequences in Table 4A). When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts and are identified in Table 4A. Guide RNAs are chemically synthesized by methods known in the art.

Guide RNA was chemically synthesized by commercial vendors or using standard in vitro synthesis techniques with modified nucleotides.

### Hepatocyte Cell Preparation

Primary mouse hepatocytes (PMH), primary rat hepatocytes (PRH), primary human hepatocytes (PHH), and primary cynomolgus hepatocytes (PCH) were prepared as follows. PMH (Gibco, MCM837, unless otherwise specified), PRH (Gibco, Rs977, unless otherwise specified), PCH (In Vitro ADMET Laboratories, 10136011, unless otherwise specified), PHH (Gibco, Hu8284, unless otherwise specified) were thawed and resuspended in 50 mL Cryopreserved Hepatocyte Recovery Media (CHRM) (Invitrogen, CM7000) followed by centrifugation. Cells were resuspended in hepatocyte medium with plating supplements: Williams' E Medium Plating Supplements with FBS content (Gibco, Cat. A13450). Cells were pelleted by centrifugation, resuspended in media and plated at a density of 20,000 cells/well for PMH, and 30,000 for PHH on Bio-coat collagen I coated 96-well plates (Corning #354407). Plated cells were allowed to settle and adhere for 4-6 hours in a tissue culture incubator at 37° C. and 5% CO₂ atmosphere. After incubation cells were checked for monolayer formation and were washed once and plated with 100 μL hepatocyte maintenance medium: Williams' E Medium (Gibco, Cat. A12176-01) plus supplement pack (Gibco, Cat. CM3000).

### HEK Cell Preparation

HEK-293 cells (ATCC, CRL-1573, unless otherwise specified) were thawed and resuspended in serum-free Dulbecco's Modified Eagle Medium (Corning #10-013-CV) with 10% FBS content (Gibco #A31605-02) and 1% Penicillin-Streptomycin (Gibco #15070063). Cells were counted and plated in Dulbecco's Modified Eagle Medium (Corning #10-013-CV) with 10% FBS content (Gibco #A31605-02) on 96-well tissue culture plate (Falcon, #353072). Plated cells were allowed to settle and adhere for 18 hours in a tissue culture incubator at 37° C. and 5% CO₂ atmosphere.

Preparation of LNP Formulation Containing sgRNA and Cas9 mRNA

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, distearoylphosphatidylcholine (DSPC), and 1,2-dimyristoyl-rac-glycero-3-methylpolyoxyethylene glycol 2000 (PEG2k-DMG) in a molar ratio of 50% Lipid A, 38% cholesterol, 9% DSPC, and 3% PEG2k-DMG. The LNPs were formulated with a lipid amine to RNA phosphate (N:P) molar ratio of about 6. The LNPs used comprise a single RNA species such as Cas9 mRNA or a sgRNA. LNP are similarly prepared with a mixture of Cas9 mRNA and a guide RNA.

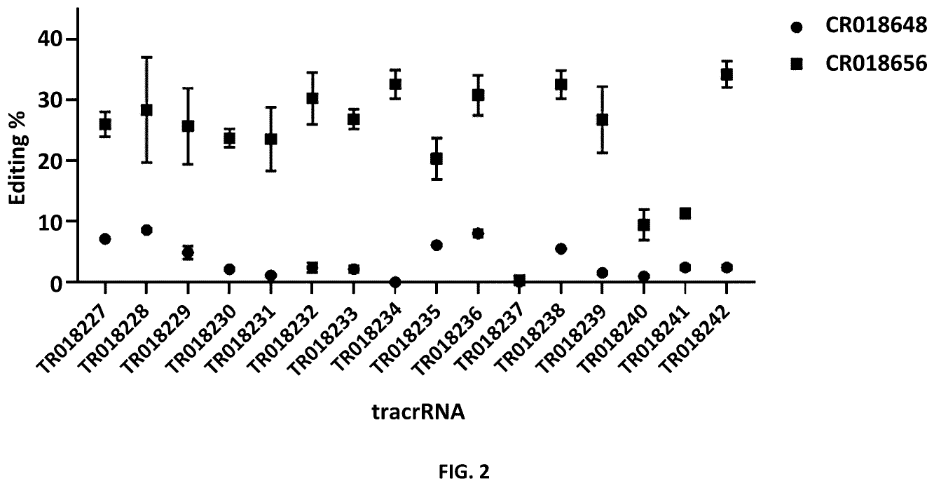

The LNPs were prepared using a cross-flow technique utilizing impinging jet mixing of the lipid in ethanol with two volumes of RNA solution and one volume of water. First, the lipid in ethanol was mixed through a mixing cross with the two volumes of RNA solution. Then, a fourth stream of water was mixed with the outlet stream of the cross through an inline tee (See WO2016010840 FIG. 2). The LNPs were held for 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 μm sterile filter. The final LNPs were characterized to determine the encapsulation efficiency, polydispersity index, and average particle size. The final LNP was stored at 4° C. or −80° C. until further use.

sgRNA and Cas9 mRNA Lipofection

Lipofection of Cas9 mRNA and gRNAs used pre-mixed lipid formulations. The lipofection reagent contained ionizable Lipid A, cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA (e.g., Cas9 mRNA and gRNA) at a lipid amine to RNA phosphate (N:P) molar ratio of about 6.0.

### Next-Generation Sequencing (“NGS”) and Analysis for Editing Efficiency

Genomic DNA was extracted using a commercial kit according to the manufacturer's protocol, for example QuickExtract™ DNA Extraction Solution (Lucigen, Cat. QE09050). To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations, or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 2. In Vitro Editing with Truncated NmeCas9 Guides in Human Embryonic Kidney (HEK) Cells

Truncated Nme-Cas9 sgRNAs targeting the SEAP gene were designed and tested to evaluate the impact of each modification on guide functionality. Modifications in guides that retained their editing efficacy were considered well-tolerated and included in future studies.

### Example 2.1 Plasmid Evaluation of sgRNA Modification Patterns

### HEK Cell Preparation

HEK-Blue™ cells, a HEK reporter cell line with a SEAP reporter, from (Invivogen, Cat. hkb-il1b) were thawed and resuspended in 15 mL Growth Media (DMEM, 4.5 g/l glucose, 2 mM L-Glutamine, 10% (v/v) fetal bovine serum (FBS), 50 U/ml penicillin, 50 mg/ml streptomycin, 100 mg/ml Normocin™ followed by centrifugation. The supernatant was discarded and the pelleted cells resuspended in Growth Media. Cells were plated at a density of 10,000 cells/well on 96-well tissue culture plate (Falcon, #353072) with Test Medium: DMEM, 4.5 g/l glucose, 2 mM L-Glutamine, 10% (v/v), heat-inactivated FBS (30 min at 56° C.), 50 U/ml penicillin, 50 mg/ml, streptomycin, 100 mg/ml Normocin™). Plated cells were allowed to settle and adhere for 18 hours in a tissue culture incubator at 37° C. and 5% CO₂ atmosphere.

### Plasmid-Based Transfection

NmeCas9 sgRNA truncation variants, with identical 23-nt complementarity to the SEAP cassette of the HEK-Blue™ cells, but with different sgRNA scaffold truncations were designed and cloned using standard Gibson Assembly methods for plasmid generation. The final plasmid reaction was plated on Luria Broth (LB) plates (Teknova, L8000) supplemented with ampicillin (Teknova, L5104) and incubated in a tissue culture incubator at 37° C. Plasmid colonies were inoculated in 4 ml Luria Broth plates (Teknova, L8000) supplemented with 100 ug/mL ampicillin (Teknova, A2135) and incubated at 37° C. overnight. Plasmids were purified according to manufacturer's instructions using Zyppy plasmid purification kit (Zymo Research, D4036).

After incubation, plasmid concentrations were measured using Qubit HS assay (Invitrogen, Catalog #Q32851) and diluted to 50 ng/μL. The plasmid DNA for each sgRNA and a pcDNA3.1 transfection plasmid encoding Nme1Cas9 (SEQ ID NO: 645) were diluted in Opti-MEM (Thermo Fisher, L3000015) and lipoplexed using Lipofectamine 3000 (Thermo Fisher, Catalog #51985091) according to manufacturer's instructions. Briefly, the lipofectamine-DNA mixture was incubated for 15 minutes at room temperature. After incubation, 10 μl of the mixture was added to HEK-Blue™ cells and incubated in a tissue culture incubator at 37° C. and 5% CO₂ atmosphere for 72 hours. Post-transfection, cells were harvested. Genomic DNA isolation and NGS analysis was performed as described in Example 1.

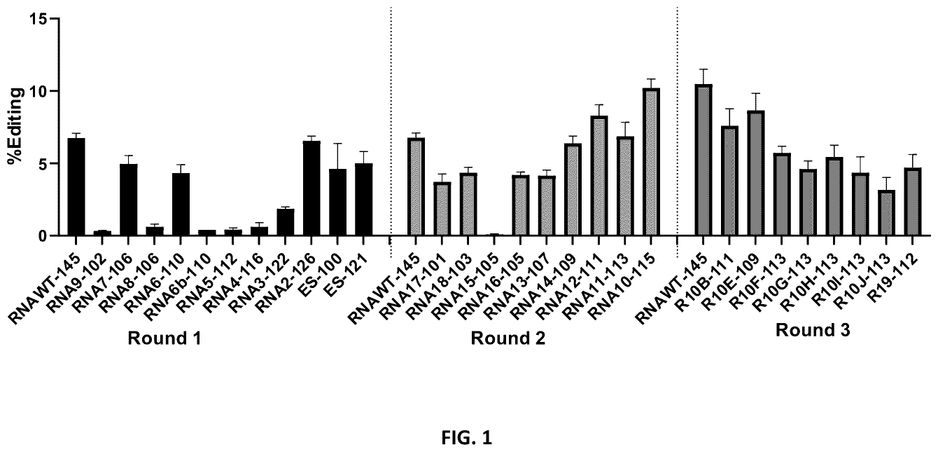

Editing efficiency was determined for unmodified sgRNA designs with identical 23-nt complementarity to the SEAP loci but with different sgRNA scaffold truncations. The assays were performed in three iterative rounds of screening to identify truncations that altered editing efficiency, either alone or in combination. Cells were prepared and analyzed using the same protocols and control plasmid (RNAWT-145). Samples were included in triplicates in each assay. Mean editing results with standard deviation (SD) are shown in Table 5 and FIG. 1. The relatively low levels of editing efficiency observed may result from the SEAP sgRNAs editing both the endogenous human SEAP gene as well as the exogenous SEAP expression cassette engineered into the HEK-Blue™ cells. NGS primers were designed such that only the editing events at the exogenous SEAP locus were quantified. Nevertheless, editing at the endogenous loci is not expected to change the relative editing levels read at the exogenous SEAP locus using truncated SEAP sgRNAs.

TABLE 5
Mean percent editing in HEK-Blue ™ cells

| Experiment | Plasmid ID | Mean % Edit | SD |
|---|---|---|---|
| Round 1 | RNAWT-145 | 6.8 | 0.3 |
| | RNA9-102 | 0.3 | 0.0 |
| | RNA7-106 | 5.0 | 0.6 |
| | RNA8-106 | 0.6 | 0.2 |
| | RNA6-110 | 4.3 | 0.6 |
| | RNA6b-110 | 0.4 | 0.0 |
| | RNA5-112 | 0.4 | 0.1 |
| | RNA4-116 | 0.6 | 0.3 |
| | RNA3-122 | 1.9 | 0.1 |
| | RNA2-126 | 6.6 | 0.3 |
| | ES-100 | 4.6 | 1.7 |
| | ES-121 | 5.0 | 0.8 |
| Round 2 | RNAWT-145 | 7.5 | 0.8 |
| | RNA17-101 | 3.7 | 0.5 |
| | RNA18-103 | 4.4 | 0.4 |
| | RNA15-105 | 0.1 | 0.0 |
| | RNA16-105 | 4.2 | 0.2 |
| | RNA13-107 | 4.1 | 0.4 |
| | RNA14-109 | 6.4 | 0.5 |
| | RNA12-111 | 8.3 | 0.8 |
| | RNA11-113 | 6.9 | 1.0 |
| | RNA10-115 | 10.2 | 0.6 |
| Round 3 | RNAWT-145 | 10.5 | 1.0 |
| | R10B-111 | 7.6 | 1.2 |
| | R10E-109 | 8.7 | 1.2 |
| | R10F-113 | 5.7 | 0.5 |
| | R10G-113 | 4.6 | 0.6 |
| | R10H-113 | 5.4 | 0.8 |
| | R10I-113 | 4.4 | 1.1 |
| | R10J-113 | 3.2 | 0.9 |
| | R19-112 | 4.7 | 0.9 |

### Example 2.2. Evaluation of gRNA Chemical Modifications in HEK-293 Cells

Guide modification patterns of select truncated sgRNA tested in the study described above were further evaluated to assess the impact of the modifications on guide editing efficiency. A stable cell line expressing Nme2 Cas9 from a lentiviral expression construct, referred to herein as HEK-Nme2, was engineered for constitutive Nme2 Cas9 expression.

### HEK Cell Preparation

HEK-293 (ATCC, CRL-1573) cells were thawed in maintenance media (DMEM (Corning, #10-013-CV), 10% FBS (Gibco, #A31605-02)). Cells were then plated at a cell density of 200,000 cells per well in 6-well plates (Corning, #353046) in Dulbecco's Modified Eagle Medium (Corning #10-013-CV) with 10% FBS content (Gibco, #A31605-02). Cells were transduced in the presence of polybrene (Millipore Sigma, TR-1003) following the manufacturer's protocol with Cellecta #SVCRU617-L lentiviral vector encoding Nme2 Cas9 (SEQ ID NO: 640).

Cells were incubated for 10 days in a tissue culture incubator at 37° C. and 5% CO₂ atmosphere. Cells were subsequently washed, processed on a cell sorter (Sony Biotechnologies, SH800Z) and analyzed using the FlowJo software package for GFP luminescence. Polyclonal mixtures of the selected HEK-Nme2 cells prepared with a MOI of 2 were used for subsequent studies.

Transduced HEK-Nme2 cells described above were thawed and resuspended in serum-free Dulbecco's Modified Eagle Medium (Corning #10-013-CV) with 10% FBS content (Gibco #A31605-02) and 1% Penicillin-Streptomycin (Gibco #15070063). Cells were counted and plated at a density of 20,000 cells/well in Dulbecco's Modified Eagle Medium (Corning #10-013-CV) with 10% FBS content (Gibco #A31605-02) on 96-well tissue culture plate (Falcon, #353072). Plated cells were allowed to settle and adhere for 18 hours in a tissue culture incubator at 37° C. and 5% CO₂ atmosphere.

### Cell Transfection Using MessengerMAX

Guide modification patterns consisting of 2′-O methyl (2′-OMe) and phosphorothioate (PS) modifications were tested in the context of sgRNA to evaluate the impact of the modifications on guide editing efficiency.

Truncated dual guide RNAs (dgRNA) were created by annealing modified, truncated tracrRNAs to modified crRNA targeting one of two sites on VEGFA (T25 or T47, target sites previously published in WO2019094791) in a mixture of 1 μl 100 μM crRNA, 1 μL 100 μM tracrRNA and 8 uL Duplex Buffer (Integrated DNA Technologies, #11-05-01-12). The Nme2-Cas9 tracrRNA and crRNA solution was annealed at 95° C. for 3 minutes followed by an incremental temperature decrease of 0.1 C/s to 20° C. Samples were kept on ice until used. Dual guide RNA with an initial concentration of 10 uM was diluted in Opti-MEM (Thermo Fisher, #51985091) for a concentration of 250 nM in 10 uL and mixed with Lipofectamine MessengerMAX (Invitrogen, catalog #LMRNA001) according to manufacturer instructions. A 20 uL aliquot of the solution was added to each tissue culture well for each concentration and incubated in a tissue culture incubator at 37° C. and 5% CO₂ atmosphere for 72 hours.

Post incubation, genomic DNA was isolated and NGS analysis was performed as described above.

Editing efficiency was evaluated as described in Example 1 for the dgRNA containing the 16 truncated tracrRNAs (TR0 #####) annealed to the crRNAs (CR0 #####), indicated in Table 6, targeting the two genomic sites (T25, T47) in the VEGFA gene at dgRNA concentrations of 50 nM. Duplicate samples were included in the assay. Mean editing results with standard deviation (SD) are shown in Table 6 and FIG. 2.

TABLE 6
In vitro editing in HEK-Nme2 cells

| | |
|---|---|
| VEGFA Site 1 | VEGFA Site 2 |
| (T25-CR018648) | (T47-CR018656) |

| | Mean % | | Mean % | |
|---|---|---|---|---|
| Tracr ID | Edit | SD | Edit | SD |
| TR018227 | 7.1 | 0.1 | 26.0 | 2.1 |
| TR018228 | 8.6 | 0.2 | 28.4 | 8.7 |
| TR018229 | 4.9 | 1.1 | 25.7 | 6.3 |
| TR018230 | 2.1 | 0.1 | 23.7 | 1.5 |
| TR018231 | 1.1 | 0.2 | 23.6 | 5.2 |
| TR018232 | 2.4 | 0.8 | 30.3 | 4.3 |
| TR018233 | 2.2 | 0.6 | 26.8 | 1.6 |
| TR018234 | 0.0 | 0.0 | 32.6 | 2.4 |
| TR018235 | 6.1 | 0.2 | 20.3 | 3.4 |
| TR018236 | 8.0 | 0.6 | 30.8 | 3.3 |
| TR018237 | 0.1 | 0.1 | 0.3 | 0.1 |
| TR018238 | 5.5 | 0.0 | 32.6 | 2.3 |
| TR018239 | 1.6 | 0.1 | 26.8 | 5.5 |
| TR018240 | 1.0 | 0.2 | 9.4 | 2.5 |
| TR018241 | 2.4 | 0.4 | 11.3 | 0.5 |
| TR018242 | 2.4 | 0.4 | 34.3 | 2.2 |

### Example 3. In Vitro Editing with Chemically Modified Nme2Cas9 sgRNAs

### Example 3.1 Evaluation of Modified sgRNA in HEK-Nme2 Cells

Guide modification patterns consisting of 2′-O methyl (2′-OMe) and phosphorothioate (PS) modifications were tested in the context of sgRNA to evaluate the impact of the modifications on guide editing efficiency.

### HEK Cell Preparation

HEK-Nme2 cells were prepared as described in Example 2. Cells were counted and plated at a density of 30,000 cells/well (Falcon, #353072). Cells were transfected as described in Examples 2.2. Seventy-two hours post transfection, the cells were prepared for NGS analysis as described in Example 1.

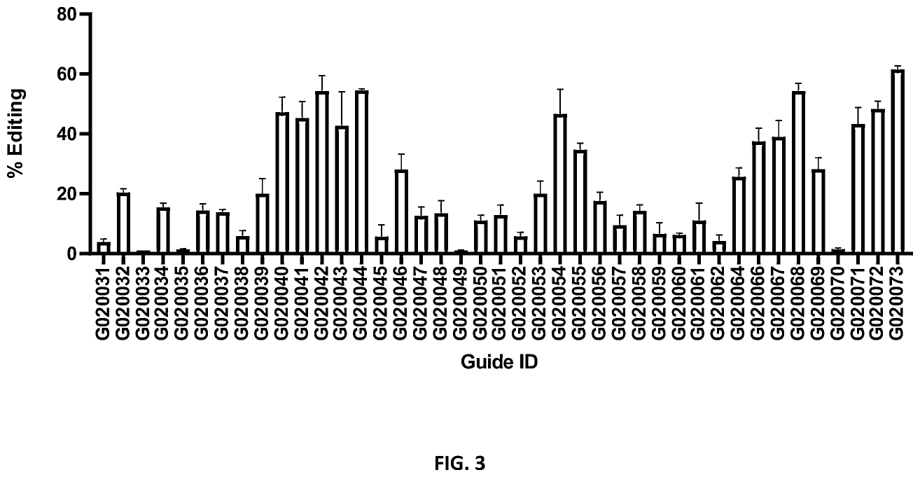

Editing efficiency was evaluated for 43 chemically modified sgRNA targeting the VEGFA gene at site T47 as described in Example 2.2. Two separate experiments were conducted with samples tested in triplicates in each assay. The results obtained in both experiments were comparable so the mean editing results with standard deviation (SD) from a single experiment are shown in Table 7 and FIG. 3. “ND” in the table represents values that could not be determined due to experimental failure.

TABLE 7
Mean percent editing in HEK-Nme2 cells

| Guide ID | Mean % Edit | SD |
|---|---|---|
| G020031 | 3.9 | 1.0 |
| G020032 | 20.4 | 1.3 |
| G020033 | 1.1 | 0.0 |
| G020034 | 15.5 | 1.4 |
| G020035 | 1.4 | 0.2 |
| G020036 | 14.4 | 2.3 |
| G020037 | 13.8 | 0.9 |
| G020038 | 5.9 | 1.8 |
| G020039 | 20.0 | 5.0 |
| G020040 | 47.2 | 5.0 |
| G020041 | 45.3 | 5.5 |
| G020042 | 54.3 | 5.1 |
| G020043 | 42.7 | 11.3 |
| G020044 | 54.5 | 0.5 |
| G020045 | 5.7 | 3.9 |
| G020046 | 28.1 | 5.1 |
| G020047 | 12.6 | 3.0 |
| G020048 | 13.5 | 4.2 |
| G020049 | 1.1 | 0.1 |
| G020050 | 11.1 | 1.7 |
| G020051 | 12.9 | 3.3 |
| G020052 | 5.8 | 1.3 |
| G020053 | 20.0 | 4.2 |
| G020054 | 46.6 | 8.2 |
| G020055 | 34.7 | 2.2 |
| G020056 | 17.6 | 2.9 |
| G020057 | 9.5 | 3.3 |
| G020058 | 14.3 | 2.0 |
| G020059 | 6.6 | 3.7 |
| G020060 | 6.3 | 0.5 |
| G020061 | 11.1 | 5.8 |
| G020062 | 4.2 | 2.0 |
| G020063 | ND | ND |
| G020064 | 25.7 | 2.9 |
| G020065 | ND | ND |
| G020066 | 37.5 | 4.4 |
| G020067 | 39.0 | 5.5 |
| G020068 | 54.3 | 2.5 |
| G020069 | 28.2 | 3.8 |
| G020070 | 1.5 | 0.4 |
| G020071 | 43.3 | 5.5 |
| G020072 | 48.3 | 2.6 |
| G020073 | 61.5 | 1.2 |

### Example 3.2 Evaluation of Alternative sgRNA Modification Patterns in HEK-293 Cells

Additional Nme2 sgRNAs with alternative modification patterns were tested in HEK-293 cells (ATCC, CRL-1573) to evaluate the impact of additional chemical modifications on guide editing efficiency. Cells were prepared and transfected as described in Example 1, delivering 100 ng Nme2 mRNA and sgRNA at a final concentration of 50 nM. Cells were plated at a density of 15,000 cells/well. Seventy-two hours post transfection, genomic DNA was isolated and analyzed via NGS as described in Example 1.

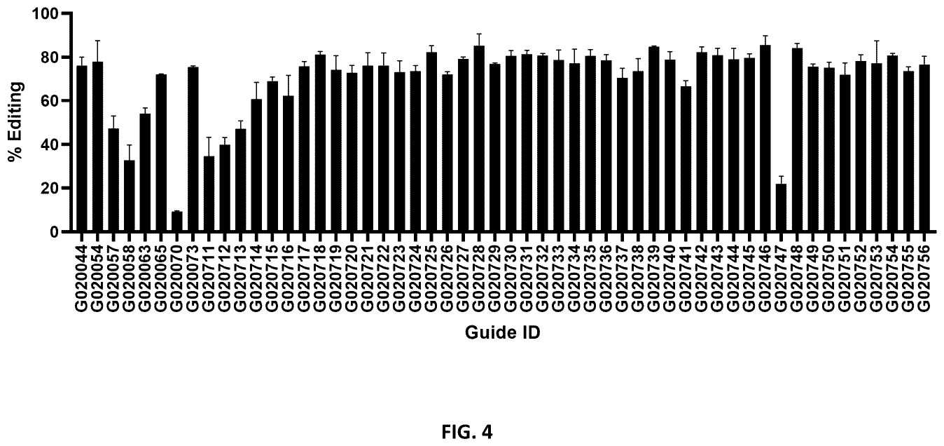

Editing efficiency was evaluated for 54 chemically modified sgRNAs targeting the VEGFA gene at site T47 as provided above. Triplicate samples were tested in each assay. Mean editing results with standard deviation (SD) are shown in Table 8 and FIG. 4.

TABLE 8
Mean percent editing in HEK-293 cells

| Guide ID | Mean % Edit | SD |
|---|---|---|
| G020044 | 76.2 | 3.9 |
| G020054 | 78.1 | 9.4 |
| G020057 | 47.3 | 5.8 |
| G020058 | 32.8 | 7.1 |
| G020063 | 54.1 | 2.7 |
| G020065 | 72.2 | 0.2 |
| G020070 | 9.3 | 0.3 |
| G020073 | 75.5 | 0.6 |
| G020711 | 34.7 | 8.6 |
| G020712 | 39.9 | 3.3 |
| G020713 | 47.2 | 3.6 |
| G020714 | 60.7 | 7.7 |
| G020715 | 69.0 | 1.9 |
| G020716 | 62.2 | 9.5 |
| G020717 | 75.9 | 2.2 |
| G020718 | 81.3 | 1.4 |
| G020719 | 74.3 | 6.4 |
| G020720 | 73.0 | 3.4 |
| G020721 | 76.1 | 5.9 |
| G020722 | 76.2 | 5.7 |
| G020723 | 73.3 | 5.1 |
| G020724 | 73.7 | 2.5 |
| G020725 | 82.4 | 2.9 |
| G020726 | 72.2 | 1.2 |
| G020727 | 79.3 | 0.8 |
| G020728 | 85.3 | 5.3 |
| G020729 | 76.9 | 0.5 |
| G020730 | 80.6 | 2.4 |
| G020731 | 81.4 | 1.8 |
| G020732 | 80.7 | 1.0 |
| G020733 | 78.7 | 4.6 |
| G020734 | 77.3 | 6.3 |
| G020735 | 80.6 | 2.8 |
| G020736 | 78.5 | 2.7 |
| G020737 | 70.4 | 4.6 |
| G020738 | 73.7 | 5.6 |
| G020739 | 84.8 | 0.4 |
| G020740 | 78.9 | 3.7 |
| G020741 | 66.6 | 2.5 |
| G020742 | 82.4 | 2.4 |
| G020743 | 80.9 | 3.1 |
| G020744 | 79.0 | 5.0 |
| G020745 | 79.7 | 1.9 |
| G020746 | 85.5 | 4.3 |
| G020747 | 21.9 | 3.4 |
| G020748 | 84.2 | 2.0 |
| G020749 | 75.7 | 1.2 |
| G020750 | 75.2 | 2.5 |
| G020751 | 72.0 | 5.4 |
| G020752 | 78.4 | 2.7 |
| G020753 | 77.3 | 10.2 |
| G020754 | 80.7 | 1.1 |
| G020755 | 73.6 | 2.0 |
| G020756 | 76.5 | 4.0 |

### Example 4. In Vitro Editing with Selected Guides in Primary Mouse Hepatocytes (PMH)

A modified sgRNA screen was conducted to evaluate the editing efficiency of 95 different sgRNAs targeting various sites within the mouse TTR gene. Based on that study, two sgRNAs (G021320 and G021256) were selected for evaluation in a dose response assay. These two test guides were compared to a mouse TTR SpyCas9 guide (G000502) with a 20 nucleotide guide sequence. The tested NmeCas9 sgRNAs targeting the mouse TTR gene include a 24 nucleotide guide sequence (as represented by N) and a guide scaffold as follows: mN*mNNNNNNNNmNNNmNNNNNNNNNNmGUUGmUmAmGmCUCCCmUmGm AmAmAmCmCGUUmGmCUAmCAAU*AAGmGmCCmGmUmCmGmAmAmAmGmAm UGUGCmCGCmAmAmCmGCUCUmGmCCmUmUmCmUGmGCmAmUC*mG*mU*mU (SEQ ID NO: 4), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guides are provided in Table 1-2.

Guides and Cas9 mRNA were lipofected, as described below, into primary mouse hepatocytes (PMH). PMH (In Vitro ADMET Laboratories MCM114) were prepared as described in Example 1. Lipofections were performed as described in Example 1 with a dose response of sgRNA and mRNA. Briefly, cells were incubated at 37° C., 5% CO₂ for 24 hours prior to treatment with lipoplexes. Lipoplexes were incubated in maintenance media containing 10% fetal bovine serum (FBS) at 37° C. for 10 minutes. Post-incubation the lipoplexes were added to the mouse hepatocytes in an 8 point, 3-fold dose response assay starting at maximum dose of 300 ng Cas9 mRNA and 50 nM sgRNA. Messenger RNA doses scale along with gRNA dose in each condition, although only gRNA dose is listed in Table 9. The cells were lysed 72 hours post-treatment and NGS analysis was performed as described in Example 1.

Dose response of editing efficiency to guide concentration was performed in triplicate samples. Table 9 shows mean percent editing and standard deviation (SD) at each guide concentration and a calculated EC₅₀ value. Mean and standard deviation (SD) is illustrated in FIG. 5.

TABLE 9
Mean percent editing in primary mouse hepatocytes

| | EC₅₀ | SgRNA | Mean % | |
|---|---|---|---|---|
| Sample | (nM) | (nM) | Edit | SD |
| SpyCas9 mRNA + | 22.0 | 50 | 95.7 | 0.3 |
| G000502 | | 16.7 | 40.9 | 14.8 |
| | | 5.6 | 6.4 | 3.3 |
| | | 1.9 | 0.8 | 0.3 |
| | | 0.6 | 0.2 | 0.08 |
| | | 0.2 | 0.1 | 0 |
| | | 0.1 | 0.1 | 0 |
| | | 0 | 0.1 | 0 |
| Nme2 Cas9 | 18.7 | 50 | 86.5 | 0.9 |
| mRNA Q (SEQ | | 16.7 | 41.8 | 2.1 |
| ID NO: 635) + | | 5.6 | 5.8 | 1.4 |
| G021320 | | 1.9 | 1.2 | 0.3 |
| | | 0.6 | 0.4 | 0.2 |
| | | 0.2 | 0.1 | 0.1 |
| | | 0.1 | 0.1 | 0 |
| | | 0 | 0.1 | 0 |
| Nme2 Cas9 | 20.9 | 50 | 92.3 | 0.5 |
| mRNA Q (SEQ | | 16.7 | 35.1 | 2 |
| ID NO: 635) + | | 5.6 | 2.6 | 0.5 |
| G021256 | | 1.9 | 0.6 | 0.4 |
| | | 0.6 | 0.1 | 0 |
| | | 0.2 | 0.1 | 0 |
| | | 0.1 | 0.1 | 0 |
| | | 0 | 0.1 | 0 |

### Example 5. In Vitro Editing in Primary Mouse Hepatocytes (PMH) with Dilution Curve

### Example 5.1. Modified sgRNA Evaluation Using Dilution Series

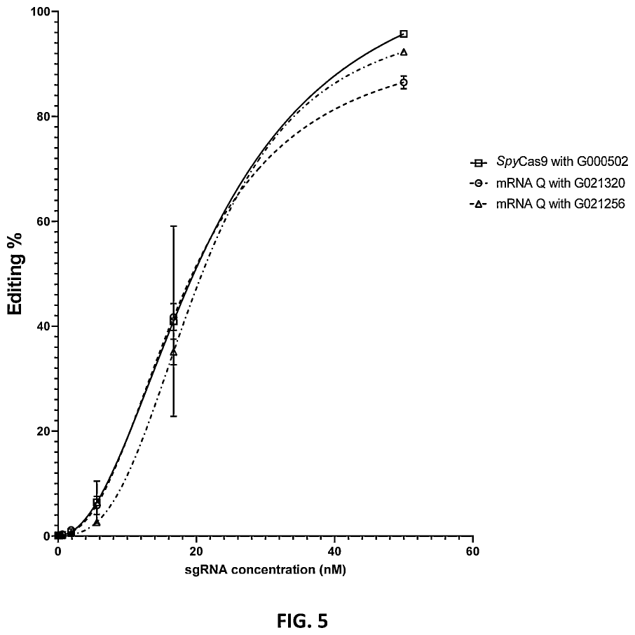

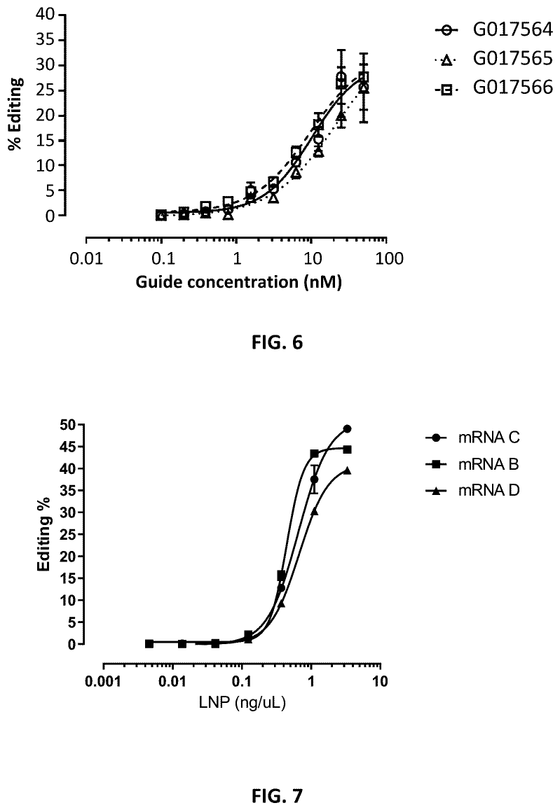

Modified sgRNAs with various scaffold structures, all targeting a previously published site in the mouse pcsk9 gene (see WO2019094791) were designed as shown in Tables 1-2 and tested for editing efficiency using in primary mouse hepatocytes (PMH). Cells were prepared as described in Example 1 using PMH cells (In Vitro ADMET Laboratories) and plated at a density of 20,000 cells/well. Cells were transfected using MessengerMax (Invitrogen) according to the manufacturer's protocols with 1 ng/μl Nme2 Cas9 mRNA (mRNA U) and sgRNA at concentrations as indicated in Table 10. Duplicate samples were included in the assay. Cells were harvested 72 hours following transfection and analyzed by NGS as described in Example 1. Mean percent editing with standard deviation are shown in Table 10 and FIG. 6.

TABLE 10
Mean percent editing in PMH

| Guide | G017564 | | G017565 | | G017566 | |
|---|---|---|---|---|---|---|
| concentration | Mean % | | Mean % | | Mean % | |
| (nM) | editing | SD | editing | SD | editing | SD |
| 50.0 | 25.7 | 3.2 | 25.6 | 4.9 | 27.7 | 0.4 |
| 25.0 | 27.8 | 1.4 | 20.0 | 1.7 | 26.4 | 4.7 |
| 12.5 | 15.3 | 1.7 | 12.9 | 0.8 | 18.2 | 1.7 |
| 6.3 | 10.6 | 0.6 | 8.7 | 0.9 | 12.6 | 0.9 |
| 3.1 | 5.3 | 0.2 | 3.6 | 0.3 | 6.7 | 0.7 |
| 1.6 | 5.0 | 1.1 | 3.6 | 0.6 | 4.8 | 0.2 |
| 0.8 | 1.3 | 0.5 | 0.2 | 0.1 | 2.8 | 0.3 |
| 0.4 | 0.8 | 0.3 | 0.5 | 0.1 | 1.8 | 0.1 |
| 0.2 | 0.3 | 0.1 | 0.2 | 0.1 | 0.7 | 0.1 |
| 0.1 | 0.1 | 0.0 | 0.1 | 0.1 | 0.2 | 0.1 |
| 0.0 | 0.1 | 0.0 | 0.0 | 0.0 | 0.3 | 0.1 |

### Example 5.2. Evaluation of mRNA Poly-A Tail Modifications and Cargo Ratios

An sgRNA targeting the mouse pcsk9 gene was selected from Table 10 to evaluate guide editing efficiency resulting from particular combinations of poly-A tail modifications and sgRNA:mRNA ratios. PMH cells used were prepared, treated, and analyzed as described in Example 1 unless otherwise noted. PMH (Gibco) were plated at a density of 15,000 cells/well.

LNPs were generally prepared as described in Example 1. LNPs were prepared with the lipid composition of 50/9/38/3, expressed as the molar ratio of ionizable lipid A/cholesterol/DSPC/PEG, respectively. The LNPs were formulated with a lipid amine to RNA phosphate (N:P) molar ratio of about 6. LNPs encapsulated gRNA G017566 or one of three mRNAs encoding the same Nme2Cas9 open reading frame (ORF) but with different encoded poly-A tails, as indicated in Table 11. A preliminary experiment holding the sgRNA application constant and varying the amount of mRNA applied showed that 1:1 sgRNA:mRNA ratio by weight resulted in the highest percent editing. In the current Example, increasing doses mRNA LNP and gRNA LNP were applied to cells in 100 μl media as described in Table 11, maintaining a 1:1 sgRNA:mRNA ratio by weight. Table 11 and FIG. 7 show mean percent editing and standard deviation (SD).

TABLE 11
Mean percent editing in PMH

| | | | |
|---|---|---|---|
| | mRNA C | mRNA B | mRNA D |
| | SEQ ID | SEQ ID | SEQ ID |
| Total | NO: 622 | NO: 621 | NO: 623 |

| RNA | Mean % | | Mean % | | Mean % | |
|---|---|---|---|---|---|---|
| (ng) | editing | SD | editing | SD | editing | N |
| 333. | 49.1 | 0.9 | 44.3 | 0.0 | 39.6 | 1 |
| 111. | 37.5 | 3.2 | 43.4 | 0.2 | 30.3 | 1 |
| 37. | 12.8 | 0.2 | 15.6 | 1.0 | 9.3 | 1 |
| 12.3 | 1.3 | 0.2 | 2.2 | 0.0 | 1.1 | 1 |
| 4.1 | 0.1 | 0.0 | 0.2 | 0.0 | 0.0 | 1 |
| 1.4 | 0.1 | 0.0 | 0.0 | 0.0 | 0.1 | 1 |
| 0.5 | 0.1 | 0.0 | 0.1 | 0.1 | 0.0 | 1 |

### Example 5.3. sgRNA:mRNA Ratio Relative to sgRNA or pgRNA Using LNPs

Studies were conducted to evaluate the editing efficiency of sgRNA designs that contain PEG linkers (pgRNA). The study compared two gRNAs targeting TTR with the same guide sequence, one of which included three PEG linkers in the constant region of the guide (pgRNA, G021846) and one of which did not (G021845) as shown in Table 12. The guides and mRNA were formulated in separate LNPs and mixed to the desired ratios for delivery to primary mouse hepatocytes (PMH) via lipid nanoparticles (LNPs).

PMH cells were prepared, treated, and analyzed as described in Example 1 unless otherwise noted. PMH cells from In Vitro ADMET Laboratories (Lot #MCM114) were plated at a density of 15,000 cells/well. Cells were treated with LNPs as described below. LNPs were generally prepared as described in Example 1. LNPs were prepared with the lipid composition of 50/9/38/3, expressed as the molar ratio of ionizable lipid A/cholesterol/DSPC/PEG, respectively. The LNPs were formulated with a lipid amine to RNA phosphate (N:P) molar ratio of about 6. LNPs encapsulated a single RNA species, either gRNA G021845, gRNA G021846 or mRNA (mRNA M; SEQ ID NO: 631) as described in Example 1.

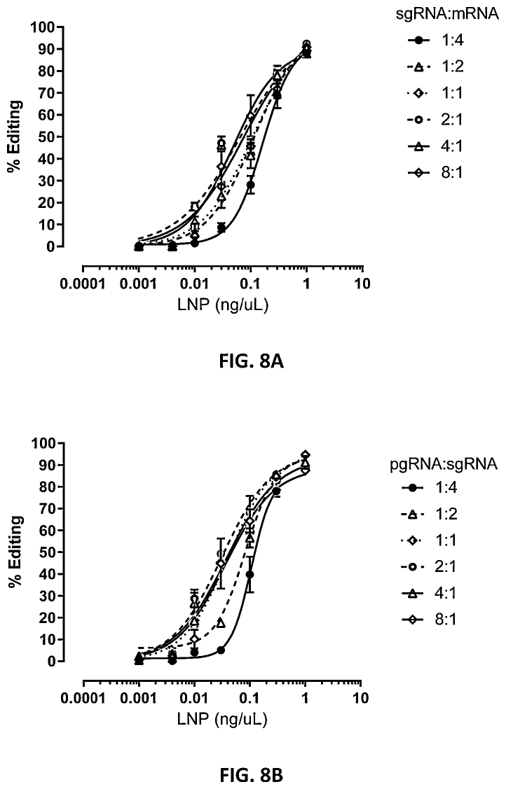

PMH cells were treated with varying amounts of LNPs at ratios of gRNA to mRNA of 1:4, 1:2, 1:1, 2:1, 4:1, or 8:1 by weight of RNA cargo. Duplicate samples were included in each assay. Guides were assayed in an 8 point 3-fold dose response curve starting at 1 ng/uL total RNA concentration as shown in Table 12. Mean percent editing results are shown in Table 12. FIG. 8A shows mean percent editing for sgRNA G021845 and FIG. 8B shows mean percent editing for sgRNA G021846. “ND” in the table represents values that could not be detected due to experimental failure.

TABLE 12
Mean percent editing in PMH

| | | sgRNA | | pgRNA | |
|---|---|---|---|---|---|
| | | (G021845) | | (G021846) | |
| Cargo ratio | LNP dose | Mean % | | Mean % | |
| (gRNA:mRNA) | (ng/uL) | editing | SD | editing | SD |
| 1:4 | 1 | 88.1 | 1.7 | ND | ND |
| | 0.3 | 68.7 | 5.7 | 78 | 0.3 |
| | 0.1 | 28.1 | 4.1 | 39.8 | 8.2 |
| | 0.03 | 8.7 | 2 | 5.1 | 0 |
| | 0.01 | 1.5 | 0.4 | 4 | 1.2 |
| | 0.004 | 0.6 | 0.5 | 0.2 | 0 |
| | 0.001 | 0.3 | 0.2 | 0.6 | 0.3 |
| 1:2 | 1 | 90.6 | 0 | 91.2 | 2.9 |
| | 0.3 | 78 | 2.4 | 85.6 | 1.4 |
| | 0.1 | 41.5 | 5.8 | 56.6 | 4.4 |
| | 0.03 | 23 | 5.4 | 17.5 | 0 |
| | 0.01 | 6.1 | 4.3 | 18.6 | 0.5 |
| | 0.004 | 0.1 | 0.1 | 3.4 | 1.7 |
| | 0.001 | 0.1 | 0 | 2.4 | 0.7 |
| 1:1 | 1 | 90.9 | 1.4 | 94.7 | 0.6 |
| | 0.3 | 71.8 | 4.2 | 84.7 | 0.9 |
| | 0.1 | 45.7 | 3.2 | 64.3 | 5.3 |
| | 0.03 | 27.4 | 1 | 44.8 | 11.5 |
| | 0.01 | 4.7 | 2.5 | 10.2 | 4.3 |
| | 0.004 | 0.2 | 0 | 1.7 | 0.7 |
| | 0.001 | 0.1 | 0 | 0.7 | 0.5 |
| 2:1 | 1 | 92.4 | 1.6 | 94.5 | 0.8 |
| | 0.3 | 80 | 1.3 | 85.7 | 0.2 |
| | 0.1 | 45.4 | 0 | 68 | 7.9 |
| | 0.03 | 47.2 | 3 | 49.3 | 0 |
| | 0.01 | 18.1 | 1.8 | 28.8 | 4.1 |
| | 0.004 | 0.8 | 0.7 | 3.8 | 2.4 |
| | 0.001 | 0.2 | 0.1 | 0.8 | 0.3 |
| 4:1 | 1 | 87.9 | 1.9 | 90.1 | 0 |
| | 0.3 | 80.2 | 2.2 | 84 | 0.1 |
| | 0.1 | 43.4 | 0 | 60.4 | 0.1 |
| | 0.03 | 46.2 | 0.5 | 46.1 | 0 |
| | 0.01 | 11.3 | 2.3 | 26.7 | 4.9 |
| | 0.004 | 0.4 | 0.2 | 1.5 | 0.4 |
| | 0.001 | 0.4 | 0.1 | 0.5 | 0.3 |
| 8:1 | 1 | 89.2 | 0 | 87.5 | 0 |
| | 0.3 | 76.7 | 3.9 | 78.6 | 3.1 |
| | 0.1 | 59.5 | 9.4 | 59.4 | 1.1 |
| | 0.03 | 36.4 | 7 | 45.3 | 0.5 |
| | 0.01 | 8.2 | 1.2 | 18.7 | 2.9 |
| | 0.004 | 0.6 | 0.6 | 2.6 | 0.3 |
| | 0.001 | 0.1 | 0 | 0.6 | 0.2 |

### Example 5.4 In Vitro Editing of Modified Pegylated Guides (pgRNAs) in PMH Using LNPs

Modified pgRNA Having the Same Targeting Site in the Mouse TTR Gene were Assayed to Evaluate the Editing Efficiency in PMH Cells.

PMH cells were prepared, treated, and analyzed as described in Example 1 unless otherwise noted. PMH cells from In Vitro ADMET Laboratories (Lot #MC148) were used and plated at a density of 15,000 cells/well. LNP formulations were prepared as described in Example 1. LNPs were prepared with the lipid composition of 50/9/38/3, expressed as the molar ratio of ionizable lipid A/cholesterol/DSPC/PEG, respectively. The LNPs were formulated with a lipid amine to RNA phosphate (N:P) molar ratio of about 6 and a gRNA indicated in Table I or mRNA

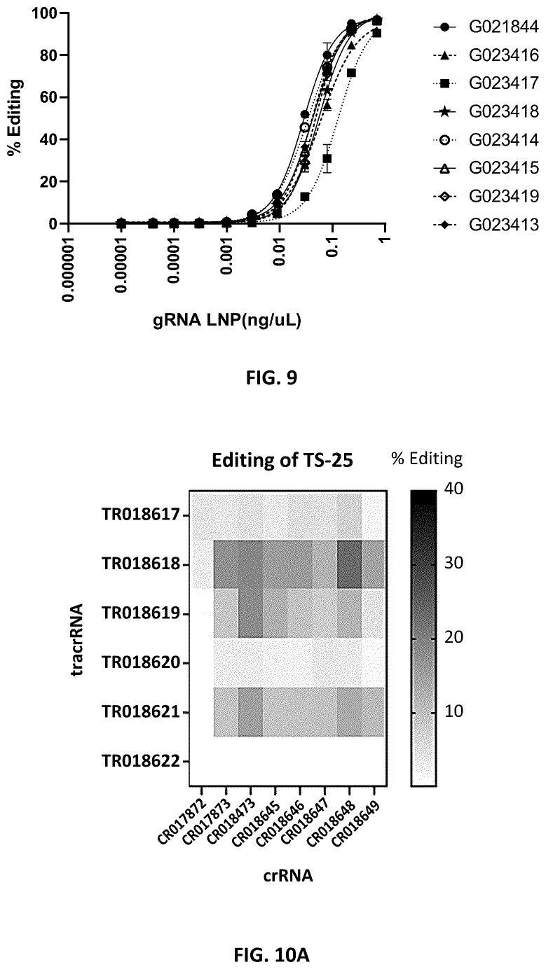

PMH in 100 μl media were treated with LNP for 30 ng total mRNA (mRNA P). by weight and LNP for gRNA in the amounts indicated in Table 13. Samples were run in duplicate. Mean editing results for PMH are shown in Table 13. and in FIG. 9.

TABLE 13
Mean percent editing in PMH

| | LNP | Mean | |
|---|---|---|---|
| | sgRNA | % | |
| Guide ID | (ng/uL) | editing | SD |
| G021844 | 0.7 | 96.6 | 0.5 |
| | 0.23 | 95.0 | 0.5 |
| | 0.08 | 80.0 | 5.9 |
| | 0.03 | 51.9 | 1.9 |
| | 0.009 | 13.9 | 0.4 |
| | 0.003 | 4.6 | 0.9 |
| | 0.001 | 0.8 | 0.1 |
| | 0.0003 | 0.2 | 0.1 |
| | 0.0001 | 0.1 | 0.0 |
| | 0.00004 | 0.2 | 0.1 |
| | 0.00001 | 0.1 | 0.0 |
| | 0 | 0.1 | 0.0 |
| G023413 | 0.7 | 96.4 | 0.4 |
| | 0.23 | 92.5 | 0.7 |
| | 0.08 | 73.9 | 0.9 |
| | 0.03 | 36.4 | 2.6 |
| | 0.009 | 10.3 | 1.5 |
| | 0.003 | 2.4 | 0.7 |
| | 0.001 | 0.6 | 0.1 |
| | 0.0003 | 0.3 | 0.0 |
| | 0.0001 | 0.1 | 0.0 |
| | 0.00004 | 0.1 | 0.0 |
| | 0.00001 | 0.1 | 0.0 |
| | 0 | 0.1 | 0.0 |
| G023414 | 0.7 | 96.5 | 0.2 |
| | 0.23 | 92.7 | 0.4 |
| | 0.08 | 74.1 | 2.7 |
| | 0.03 | 45.7 | 1.5 |
| | 0.009 | 13.7 | 0.7 |
| | 0.003 | 4.3 | 1.3 |
| | 0.001 | 0.7 | 0.1 |
| | 0.0003 | 0.2 | 0.0 |
| | 0.0001 | 0.2 | 0.1 |
| | 0.00004 | 0.2 | 0.1 |
| | 0.00001 | 0.1 | 0.0 |
| | 0 | 0.1 | 0.0 |
| G023415 | 0.7 | 96.5 | 0.5 |
| | 0.23 | 92.6 | 0.7 |
| | 0.08 | 73.1 | 0.2 |
| | 0.03 | 34.4 | 0.8 |
| | 0.009 | 14.2 | 0.2 |
| | 0.003 | 3.9 | 0.4 |
| | 0.001 | 0.5 | 0.2 |
| | 0.0003 | 0.2 | 0.0 |
| | 0.0001 | 0.2 | 0.0 |
| | 0.00004 | 0.1 | 0.0 |
| | 0.00001 | 0.1 | 0.0 |
| | 0 | 0.1 | 0.0 |
| G023416 | 0.7 | 91.5 | 1.8 |
| | 0.23 | 84.8 | 0.1 |
| | 0.08 | 56.4 | 2.7 |
| | 0.03 | 28.2 | 3.6 |
| | 0.009 | 10.0 | 1.7 |
| | 0.003 | 3.2 | 0.0 |
| | 0.001 | 0.8 | 0.3 |
| | 0.0003 | 0.2 | 0.0 |
| | 0.0001 | 0.1 | 0.0 |
| | 0.00004 | 0.1 | 0.0 |
| | 0.00001 | 0.1 | 0.0 |
| | 0 | 0.1 | 0.0 |
| G023417 | 0.7 | 90.5 | 1.8 |
| | 0.23 | 71.6 | 0.2 |
| | 0.08 | 30.9 | 6.7 |
| | 0.03 | 12.8 | 1.3 |
| | 0.009 | 4.8 | 1.5 |
| | 0.003 | 0.4 | 0.4 |
| | 0.001 | 0.2 | 0.1 |
| | 0.0003 | 0.1 | 0.0 |
| | 0.0001 | 0.1 | 0.1 |
| | 0.00004 | 0.1 | 0.0 |
| | 0.00001 | 0.1 | 0.0 |
| | 0 | 0.1 | 0.0 |
| G023418 | 0.7 | 96.8 | 0.3 |
| | 0.23 | 90.8 | 1.7 |
| | 0.08 | 63.3 | 1.8 |
| | 0.03 | 27.7 | 2.4 |
| | 0.009 | 8.8 | 0.5 |
| | 0.003 | 1.9 | 0.6 |
| | 0.001 | 0.7 | 0.2 |
| | 0.0003 | 0.2 | 0.1 |
| | 0.0001 | 0.1 | 0.0 |
| | 0.00004 | 0.1 | 0.0 |
| | 0.00001 | 0.1 | 0.0 |
| | 0 | 0.2 | 0.1 |
| G023419 | 0.7 | 96.6 | 0.6 |
| | 0.23 | 93.4 | 1.3 |
| | 0.08 | 71.1 | 3.3 |
| | 0.03 | 29.0 | 4.6 |
| | 0.009 | 9.7 | 4.1 |
| | 0.003 | 2.3 | 0.5 |
| | 0.001 | 0.4 | 0.0 |
| | 0.0003 | 0.1 | 0.0 |
| | 0.0001 | 0.2 | 0.0 |
| | 0.00004 | 0.2 | 0.0 |
| | 0.00001 | 0.1 | 0.0 |
| | 0 | 0.1 | 0.0 |

### Example 6. Chemical Modification Screens

### Example 6.1. Chemical Modification Screens in HEK-293 Cells

Editing efficiency was determined for chemically modified crRNA targeting two different VEGFA target sites (TS-25, TS-47). Each dgRNA contained a combination of a crRNA and a tracRNA with chemical modifications. Chemical modifications included phosphorothioate (PS) or 2′-O′ methyl (2′-OMe) modifications to bases at the 5′ and 3′ ends of both the crRNA and the tracrRNA (EndMod).

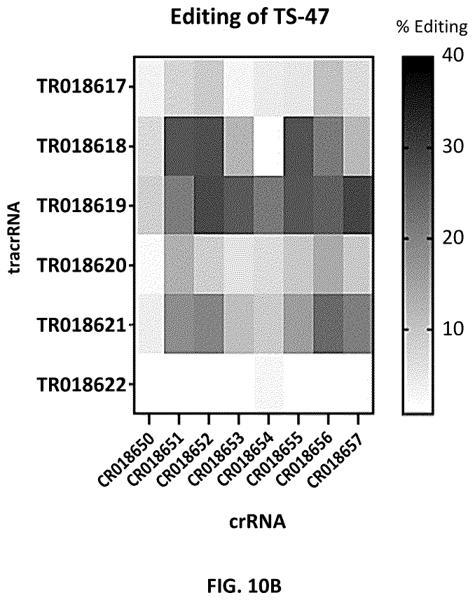

HEK-Nme2 cells were prepared as described in Example 2 except cells with MOI at 0.8 were used in this study. Cells were plated at a cell density of 10,000 cells per well. Cells were then transfected with dual guide RNA via the MessengerMax protocol described in Example 2.2 at a final concentration of 25 nM dgRNA. Duplicate samples were included in the assay. After 72 hours, genomic DNA (gDNA) was extracted from the cells and prepared for NGS analysis as described in Example 1. NGS analysis results were evaluated using the Graphpad Prism software (version 9). Mean percent editing is shown in Table 14 and FIGS. 10A-10B.

TABLE 14
Mean percent editing with different
combinations of crRNA and tracrRNA

| | Site TS-25 | | | Site TS-47 | | |
|---|---|---|---|---|---|---|
| | | Mean | | | Mean | |
| TracrRNA | crRNA | % | | crRNA | % | |
| ID | ID | Edit | SD | ID | Edit | SD |
| TR018617 | CR017872 | 4.20 | 2.20 | CR018650 | 3.25 | 0.25 |
| TR018618 | UnMod | 3.05 | 1.05 | UnMod | 6.90 | 1.50 |
| TR018619 | | 0.75 | 0.25 | | 8.55 | 2.15 |
| TR018620 | | 0.10 | 0.00 | | 1.95 | 0.05 |
| TR018621 | | 0.25 | 0.05 | | 3.80 | 1.50 |
| TR018622 | | 0.10 | 0.00 | | 1.15 | 0.75 |
| TR018617 | CR017873 | 3.80 | 2.30 | CR018651 | 7.70 | 0.10 |
| TR018618 | EndMod | 17.30 | 5.00 | EndMod | 26.70 | 3.60 |
| TR018619 | | 9.00 | 2.90 | | 20.85 | 4.25 |
| TR018620 | | 3.05 | 0.45 | | 13.15 | 3.85 |
| TR018621 | | 9.40 | 2.30 | | 18.35 | 3.55 |
| TR018622 | | 0.50 | 0.20 | | 1.20 | 0.40 |
| TR018617 | CR018473 | 4.95 | 1.75 | CR018652 | 9.10 | 2.90 |
| TR018618 | RaMod | 18.85 | 4.15 | RaMod | 27.50 | 5.40 |
| TR018619 | | 18.00 | 2.50 | | 28.45 | 3.05 |
| TR018620 | | 3.15 | 0.25 | | 8.60 | 3.60 |
| TR018621 | | 15.15 | 1.25 | | 19.60 | 3.00 |
| TR018622 | | 0.30 | 0.10 | | 1.40 | 0.10 |
| TR018617 | CR018645 | 3.35 | 1.55 | CR018653 | 2.80 | 0.40 |
| TR018618 | RA-maxPS | 15.60 | 4.80 | RA-maxPS | 12.35 | 0.85 |
| TR018619 | | 12.75 | 1.95 | | 25.95 | 1.35 |
| TR018620 | | 2.30 | 0.50 | | 5.75 | 1.45 |
| TR018621 | | 9.40 | 0.00 | | 10.95 | 2.55 |
| TR018622 | | 0.15 | 0.05 | | 0.70 | 0.70 |
| TR018617 | CR018646 | 5.20 | 1.90 | CR018654 | 4.30 | 1.70 |
| TR018618 | maxPS | 15.75 | 3.85 | maxPS | 1.70 | 0.10 |
| TR018619 | | 9.50 | 0.90 | | 21.30 | 1.70 |
| TR018620 | | 2.20 | 0.00 | | 6.60 | 2.60 |
| TR018621 | | 9.55 | 1.05 | | 9.15 | 4.65 |
| TR018622 | | 0.15 | 0.05 | | 2.55 | 1.15 |
| TR018617 | CR018647 | 4.65 | 1.75 | CR018655 | 5.00 | 0.80 |
| TR018618 | Target-2′-OMe | 12.15 | 4.25 | Target-2′-OMe | 26.85 | 2.25 |
| TR018619 | | 8.05 | 1.45 | | 26.30 | 1.00 |
| TR018620 | | 4.00 | 0.40 | | 9.55 | 0.95 |
| TR018621 | | 9.70 | 1.50 | | 15.65 | 2.85 |
| TR018622 | | 0.10 | 0.00 | | 1.25 | 0.25 |
| TR018617 | CR018648 | 7.00 | 3.10 | CR018656 | 10.50 | 0.50 |
| TR018618 | Max-2′-OMe | 23.40 | 8.00 | Max-2′-OMe | 21.10 | 1.90 |
| TR018619 | | 11.70 | 4.30 | | 25.05 | 0.65 |
| TR018620 | | 3.30 | 0.00 | | 13.90 | 0.90 |
| TR018621 | | 13.30 | 2.20 | | 23.60 | 0.70 |
| TR018622 | | 0.20 | 0.00 | | 1.25 | 0.45 |
| TR018617 | CR018649 | 1.55 | 0.05 | CR018657 | 6.55 | 0.15 |
| TR018618 | Max-2′-OMe | 14.60 | 4.10 | Max-2′-OMe | 11.65 | 0.65 |
| TR018619 | | 4.35 | 1.25 | | 29.05 | 2.35 |
| TR018620 | | 1.25 | 0.15 | | 9.40 | 1.80 |
| TR018621 | | 10.85 | 3.55 | | 20.45 | 6.65 |
| TR018622 | | 0.10 | 0.00 | | 1.70 | 0.10 |

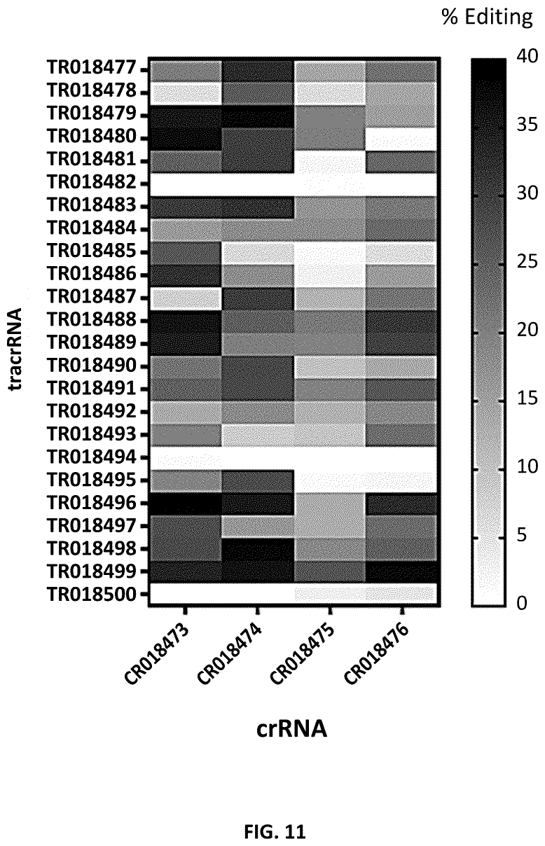

The additional combinations of chemically modified crRNA and tracrRNA were tested to assess editing efficiency. Editing efficiency was determined for chemically modified dgRNA targeting the previously described TS47 site within the VEGFA gene. Each dgRNA contained a combination of a crRNA and a tracrRNA with chemical modifications. HEK-Nme2 cells were obtained and prepared as described in Example 2. Cells (MOI=2) were plated at a cell density of 10,000 cells per well. Cells were then transfected with dual guide RNA via the MessengerMax protocol previously described in Example 2.2 at a final concentration of 25 nM dgRNA. Duplicate samples were included in the assay. After 72 hours, gDNA was extracted from cells, prepared for NGS analysis, and NGS results analyzed as described above and in Example 1. Mean percent editing is shown in Table 15 and FIG. 11.

TABLE 15
Mean percent editing with modified crRNAs and tracrRNAs

| | CR018473 | | CR018474 | | CR018475 | | CR018476 | |
|---|---|---|---|---|---|---|---|---|
| tracrRNA | Mean | | Mean | | Mean | | Mean | |
| ID | % Edit | SD | % Edit | SD | % Edit | SD | % Edit | SD |
| TR018477 | 20.5 | 0.1 | 32.8 | 1.9 | 14.2 | 4.1 | 22.2 | 2.1 |
| TR018478 | 5.8 | 1.3 | 25.6 | 4.0 | 6.1 | 0.2 | 14.2 | 0.3 |
| TR018479 | 35.7 | 2.3 | 37.9 | 2.7 | 19.9 | 1.7 | 15.4 | 0.6 |
| TR018480 | 37.4 | 0.4 | 29.7 | 0.5 | 20.2 | 1.3 | 1.2 | 0.3 |
| TR018481 | 24.9 | 1.0 | 29.9 | 0.1 | 1.9 | 0.2 | 23.6 | 0.7 |
| TR018482 | 0.3 | 0.1 | 0.5 | 0.1 | 0.9 | 0.3 | 0.8 | 0.1 |
| TR018483 | 30.3 | 1.4 | 31.8 | 1.7 | 16.9 | 0.4 | 21.1 | 3.1 |
| TR018484 | 16.2 | 1.8 | 18.2 | 1.2 | 17.9 | 1.1 | 23.2 | 3.0 |
| TR018485 | 26.1 | 0.2 | 6.6 | 1.3 | 1.5 | 0.4 | 5.9 | 1.1 |
| TR018486 | 32.0 | 4.8 | 17.9 | 0.8 | 2.8 | 0.1 | 15.7 | 0.3 |
| TR018487 | 8.0 | 1.6 | 30.1 | 1.0 | 12.2 | 0.0 | 21.8 | 0.7 |
| TR018488 | 36.2 | 0.8 | 25.1 | 1.2 | 21.0 | 1.0 | 31.0 | 0.3 |
| TR018489 | 34.9 | 0.4 | 20.1 | 1.6 | 19.5 | 0.5 | 29.4 | 1.3 |
| TR018490 | 21.9 | 0.3 | 28.5 | 0.3 | 10.2 | 1.6 | 13.8 | 1.4 |
| TR018491 | 24.7 | 1.2 | 28.3 | 1.4 | 19.6 | 1.4 | 26.1 | 0.0 |
| TR018492 | 13.6 | 1.5 | 18.3 | 0.3 | 12.4 | 3.0 | 18.7 | 1.9 |
| TR018493 | 19.9 | 0.8 | 8.4 | 2.0 | 9.6 | 1.9 | 22.7 | 3.2 |
| TR018494 | 1.1 | 0.9 | 0.4 | 0.2 | 0.3 | 0.2 | 0.3 | 0.1 |
| TR018495 | 19.4 | 1.6 | 27.8 | 0.7 | 1.4 | 0.7 | 1.9 | 0.3 |
| TR018496 | 38.7 | 0.9 | 35.5 | 1.0 | 13.3 | 1.7 | 32.9 | 1.7 |
| TR018497 | 27.5 | 1.1 | 16.6 | 0.5 | 13.1 | 0.2 | 23.0 | 0.7 |
| TR018498 | 28.0 | 1.2 | 38.1 | 1.0 | 18.6 | 2.9 | 24.9 | 1.7 |
| TR018499 | 33.9 | 0.1 | 36.4 | 0.5 | 26.6 | 0.9 | 37.8 | 1.2 |
| TR018500 | 0.7 | 0.5 | 0.5 | 0.2 | 3.1 | 0.7 | 4.7 | 1.3 |

### Example 6.2 Evaluation of Guide Sequence Chemical Modifications in PMH

Pegylated guide RNA (pgRNA) with chemical modifications in the guide sequence were tested for editing efficacy at two distinct mouse TTR regions (Exon 1 and Exon 3) in PMH. PMH (In Vitro ADMET Laboratories) were prepared as described in Example 1. Lipofection of Nme2 Cas9 mRNA (mRNA O SEQ ID NO: 633) and gRNAs targeting two distinct loci in mouse TTR as indicated in Table 16 used pre-mixed lipid compositions as described in Example 1. Lipoplexes were used to treat cells with 100 ng/100 ul Nme2 mRNA and with gRNA at the concentrations indicated in Table 16. Cells were incubated in maintenance media+10% FBS (Corning #35-010-CF) at 37° C. for 72 hours. Post incubation, genomic DNA was isolated and NGS analysis was performed as described in Example 1.

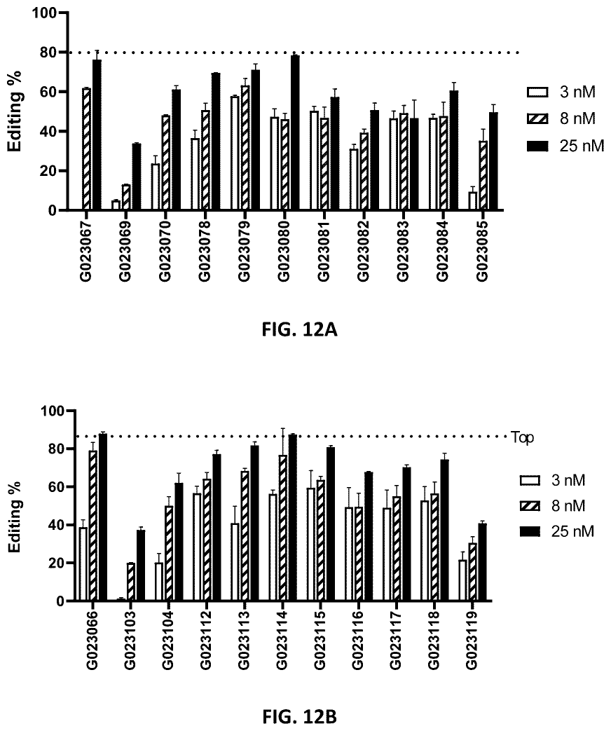

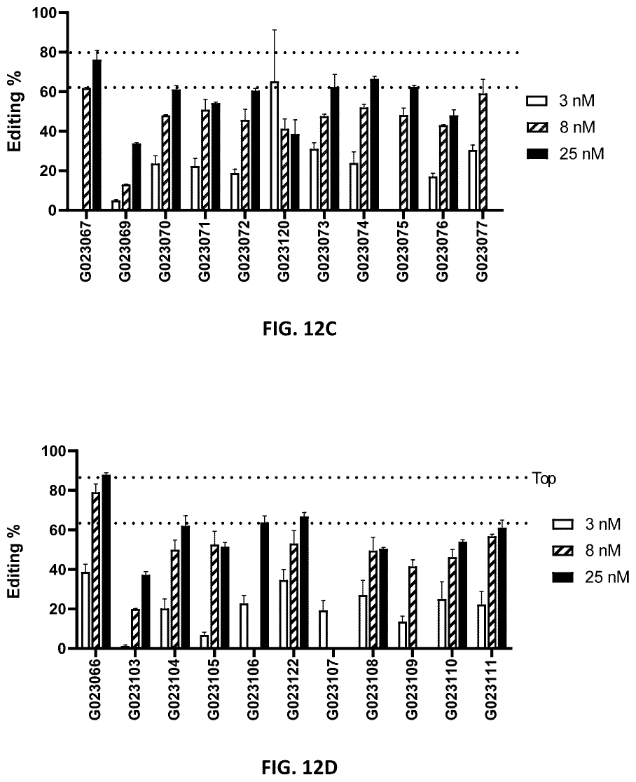

Editing efficiency was determined for various guide modification patterns at three gRNA concentrations (3 nM, 8 nM, or 25 nM). Duplicate samples were included in the assay. Mean editing results are shown in Table 16 and FIGS. 12A-12B for test guides with the N79 pgRNA design (G023066 or G023067) that are lacking a 2′-OMe at specified nucleotide position in the target-binding region of the gRNA. Table 17 and FIGS. 12C-12D show mean percent editing for test guides with the End-Mod pgRNA designs (G023070 or G023104) with additional 2′-OMe modifications at the specified nucleotide position in the target-binding region of the gRNA. “ND” in the table represents values that could not be detected due to experimental failure.

TABLE 16
Mean percent editing for N79 pgRNAs lacking 2′-OMe modification
at the specified position in the guide sequence.

| | | | gRNA concentrations | | | | | |
|---|---|---|---|---|---|---|---|---|
| | | | 3 nM | | 8 nM | | 25 nM | |
| | | | Mean | | Mean | | Mean | |
| | Guide Sequence | | % | | % | | % | |
| Locus | Modification | Guide | Edit | SD | Edit | SD | Edit | SD |
| Exon-1 | High mod pgRNA | G023067 | ND | ND | 61.8 | 0.3 | 76.3 | 4.6 |
| | No-Mod | G023069 | 5.1 | 0.4 | 13.0 | 0.2 | 33.8 | 0.3 |
| | End-Mod | G023070 | 23.7 | 3.9 | 48.1 | 0.3 | 61.2 | 2.0 |
| | POSITION 4 | G023078 | 36.5 | 4.1 | 50.7 | 3.5 | 69.5 | 0.1 |
| | POSITION 5 | G023079 | 57.7 | 0.5 | 63.2 | 3.5 | 71.2 | 3.0 |
| | POSITION 8 | G023080 | 47.3 | 4.2 | 46.1 | 2.9 | 78.4 | 0.2 |
| | POSITION 9 | G023081 | 50.4 | 2.2 | 46.8 | 5.6 | 57.4 | 4.1 |
| | POSITION 11 | G023082 | 31.2 | 2.3 | 39.3 | 1.9 | 50.7 | 3.6 |
| | POSITION 13 | G023083 | 46.5 | 3.7 | 49.2 | 3.8 | 46.6 | 9.2 |
| | POSITION 18 | G023084 | 46.8 | 1.8 | 47.7 | 7 | 60.7 | 4.1 |
| | POSITION 22 | G023085 | 9.5 | 2.6 | 35.2 | 5.8 | 49.6 | 3.9 |
| Exon-3 | High mod pgRNA | G023066 | 38.8 | 4.0 | 79.2 | 4.2 | 88.0 | 1.0 |
| | No-Mod | G023103 | 1.3 | 0.5 | 20 | 0.2 | 37.3 | 1.6 |
| | End-Mod | G023104 | 20.3 | 4.7 | 50.1 | 4.9 | 62.1 | 5.1 |
| | POSITION 4 | G023112 | 56.7 | 3.8 | 64.3 | 3.2 | 77.2 | 2.0 |
| | POSITION 5 | G023113 | 41.0 | 8.9 | 68.4 | 1.3 | 81.8 | 1.8 |
| | POSITION 8 | G023114 | 56.3 | 2.2 | 76.8 | 14 | 87.5 | 0.5 |
| | POSITION 9 | G023115 | 59.5 | 9.0 | 63.6 | 1.9 | 80.8 | 0.9 |
| | POSITION 11 | G023116 | 49.4 | 10.3 | 49.5 | 7.1 | 67.8 | 0.2 |
| | POSITION 13 | G023117 | 49.0 | 9.4 | 55.1 | 5.7 | 70.3 | 1.2 |
| | POSITION 18 | G023118 | 52.9 | 7.3 | 56.6 | 6.0 | 74.4 | 3.4 |
| | POSITION 22 | G023119 | 21.7 | 4.1 | 30.5 | 3.3 | 40.8 | 1.2 |

ND = no data reported due to technical failure.

TABLE 17
Mean percent editing for end modified pgRNAs with an additional 2′-OMe modification
at the specified position in the target-binding region of the pgRNAs.

| | | | gRNA concentrations | | | | | |
|---|---|---|---|---|---|---|---|---|
| | | | 3 nM | | 8 nM | | 25 nM | |
| | | | Mean | | Mean | | Mean | |
| | Guide Sequence | | % | | % | | % | |
| Locus | Modification | Guide | Edit | SD | Edit | SD | Edit | SD |
| Exon-1 | High mod pgRNA | G023067 | ND | ND | 61.8 | 0.3 | 76.3 | 4.6 |
| | No-Mod | G023069 | 5.1 | 0.4 | 13.0 | 0.2 | 33.8 | 0.3 |
| | End-Mod | G023070 | 23.7 | 3.9 | 48.1 | 0.3 | 61.2 | 2.0 |
| | POSITION 4 | G023071 | 22.4 | 4.0 | 51.0 | 5.3 | 54.2 | 0.6 |
| | POSITION 5 | G023072 | 18.8 | 2.1 | 45.8 | 5.3 | 60.5 | 1.2 |
| | POSITION 8 | G023120 | 65.3 | 26 | 41.3 | 5.0 | 38.6 | 7.2 |
| | POSITION 9 | G023073 | 31.1 | 3.1 | 47.7 | 1.0 | 62.4 | 6.3 |
| | POSITION 11 | G023074 | 24.0 | 5.6 | 52.0 | 1.7 | 66.5 | 1.4 |
| | POSITION 13 | G023075 | ND | ND | 48.2 | 3.6 | 62.5 | 0.8 |
| | POSITION 18 | G023076 | 17.2 | 1.6 | 43.1 | 0.2 | 48.1 | 2.8 |
| | POSITION 22 | G023077 | 30.6 | 2.5 | 59.1 | 7.2 | ND | ND |
| Exon-3 | High mod pgRNA | G023066 | 38.8 | 4.0 | 79.2 | 4.2 | 88.0 | 1.0 |
| | No-Mod | G023103 | 1.3 | 0.5 | 20.0 | 0.2 | 37.3 | 1.6 |
| | End-Mod | G023104 | 20.3 | 4.7 | 50.1 | 4.9 | 62.1 | 5.1 |
| | POSITION 4 | G023105 | 7.0 | 1.4 | 52.6 | 6.8 | 51.6 | 2.2 |
| | POSITION 5 | G023106 | 22.8 | 4.2 | ND | ND | 63.8 | 3.4 |
| | POSITION 8 | G023122 | 34.6 | 5.4 | 53.2 | 6.5 | 66.9 | 1.9 |
| | POSITION 9 | G023107 | 19.3 | 5.1 | ND | ND | ND | ND |
| | POSITION 11 | G023108 | 27.1 | 7.5 | 49.6 | 6.8 | 50.5 | 0.7 |
| | POSITION 13 | G023109 | 13.6 | 2.8 | 41.6 | 3.4 | ND | ND |
| | POSITION 18 | G023110 | 25.1 | 8.8 | 46.2 | 3.9 | 54.1 | 1.1 |
| | POSITION 22 | G023111 | 22.3 | 6.6 | 56.8 | 1.1 | 61.2 | 3.9 |

ND = no data reported due to technical failure.

### Example 7. Base Editing with Nme2-Base Editor and Chemically Modified sgRNA in HepG2 Cells

Base editor constructs comprising an APOBEC3A deaminase domain fused to Nme2Cas9 D16A nickase were tested for base conversion efficiency with various guide designs in HepG2 cells.

HepG2 cells constitutively overexpress solute carrier family 10 member 1 (SLC10A1) (HepG2-NTCP, Seeger et al. Mol Ther Nucleic Acids. 2014 December; 3(12): e216) were thawed and resuspended in Dulbecco's Modified Eagle's Medium (DMEM) supplemented with 10% Fetal Bovine Serum (FBS) (Media Y) followed by centrifugation. The supernatant was discarded and the cells were resuspended in Media Y and plated at a density of 25,000 cells per well in a 96-well collagen coated plate (Corning, Cat. 354407) in 100 uL of Media Y.

Nme2Cas9 base editor mRNAs were prepared by in vitro transcription essentially as described in Example 1 from plasmids encoding mRNA R (2XNLS N-terminal, 1xC-terminal NLS Nme2 base editor), mRNA S (2XNLS N-terminal, NLS Nme2 base editor ORF), and mRNA T (1× C-term NLS Nme2 base editor ORF). SpyCas9 mRNA and uracil glycosylase inhibitor (UGI) mRNA (SEQ ID NO: 625) were transcribed from plasmids using the same method.

Chemically modified NmeCas9 sgRNAs targeted to NTCP, with different PAM sequences, (G020927, G020928) or VEGFA (G020073) and SpyCas9 sgRNA targeted to NTCP (G020929) were synthesized using routine methods.

Guide RNA, editor mRNA, and UGI mRNA were mixed at a 1:1:1 weight ratio with premixed transfection reagent containing Lipid A, cholesterol, DSPC, and PEG2k-DMG in a 50:38:9:3 molar ratio, respectively. Reagents were combined at a lipid amine to RNA phosphate (N:P) molar ratio of about 6.0. RNA-lipid mixture was mixed approximately 1:1 with 10% FBS media and incubated for 10 minutes. Post-incubation, the cells were treated with the RNA-lipid mixture in an 8-point, 2-fold serial dilution starting at 400 ng total editor RNA per well.

At 72 hours post-treatment, cells were lysed for NGS analysis as provided in Example 1.

Dose response of editing efficiency to guide concentration was performed in triplicate. Table 18—shows mean editing percentages calculated at each guide concentration and a calculated EC50 value. The target site in VEGFA is prone to indel formation due to high GC content. All editor mRNAs achieved the same maximum C to T editing. There were slight differences in EC50 where mRNA S outperformed mRNA R and mRNA T.

TABLE 18
Mean editing percentages in HepG2-NTCP cells at
VEGFA locus (G020073) and Nme2 base editor.

| | Nme-base | C-to-T % | | | | C-to-A/G % | | | Indel % | | |
|---|---|---|---|---|---|---|---|---|---|---|---|
| mRNA | editor (ng) | Mean | SD | n | EC50 | Mean | SD | n | Mean | SD | n |
| mRNA R | 0 | 0.20 | 0.14 | 2 | 6.86 | 0.80 | 0.00 | 2 | 0.70 | 0.14 | 2 |
| | 6.25 | 36.05 | 1.63 | 2 | | 2.30 | 0.28 | 2 | 14.25 | 0.21 | 2 |
| | 12.5 | 49.70 | 1.13 | 2 | | 2.95 | 0.49 | 2 | 16.35 | 1.06 | 2 |
| | 25 | 57.60 | 0.42 | 2 | | 2.75 | 0.07 | 2 | 17.45 | 0.21 | 2 |
| | 50 | 67.15 | 1.34 | 2 | | 2.65 | 0.07 | 2 | 17.90 | 0.14 | 2 |
| | 100 | 70.50 | 0.99 | 2 | | 2.65 | 0.07 | 2 | 18.80 | 0.28 | 2 |
| | 200 | 72.75 | 0.49 | 2 | | 2.90 | 0.57 | 2 | 18.75 | 0.49 | 2 |
| | 400 | 72.00 | 0.14 | 2 | | 3.10 | 0.14 | 2 | 20.40 | 0.99 | 2 |
| mRNA S | 0 | 0.50 | 0.00 | 1 | 3.86 | 1.00 | 0.00 | 1 | 0.60 | 0.00 | 1 |
| | 6.25 | 49.60 | 3.25 | 2 | | 2.10 | 0.14 | 2 | 14.05 | 0.21 | 2 |
| | 12.5 | 57.05 | 0.78 | 2 | | 2.50 | 0.42 | 2 | 16.50 | 0.14 | 2 |
| | 25 | 66.95 | 0.49 | 2 | | 2.40 | 0.00 | 2 | 16.25 | 0.21 | 2 |
| | 50 | 71.45 | 0.35 | 2 | | 2.40 | 0.00 | 2 | 17.25 | 0.07 | 2 |
| | 100 | 71.60 | 0.85 | 2 | | 2.55 | 0.35 | 2 | 17.90 | 0.14 | 2 |
| | 200 | 73.70 | 0.28 | 2 | | 2.65 | 0.07 | 2 | 19.00 | 0.28 | 2 |
| | 400 | 73.80 | 0.14 | 2 | | 3.05 | 0.35 | 2 | 18.70 | 0.42 | 2 |
| mRNA T | 0 | 0.55 | 0.49 | 2 | 4.84 | 1.05 | 0.21 | 2 | 0.55 | 0.07 | 2 |
| | 6.25 | 45.55 | 0.21 | 2 | | 2.60 | 0.28 | 2 | 17.15 | 0.64 | 2 |
| | 12.5 | 56.40 | 1.84 | 2 | | 2.80 | 0.00 | 2 | 19.40 | 0.14 | 2 |
| | 25 | 64.95 | 1.06 | 2 | | 3.15 | 0.35 | 2 | 19.40 | 1.70 | 2 |
| | 50 | 70.70 | 0.28 | 2 | | 3.25 | 0.21 | 2 | 20.00 | 0.71 | 2 |
| | 100 | 72.20 | 2.83 | 2 | | 2.75 | 0.21 | 2 | 19.80 | 1.56 | 2 |
| | 200 | 70.90 | 0.99 | 2 | | 3.15 | 0.35 | 2 | 19.70 | 0.85 | 2 |
| | 400 | 71.80 | 0.14 | 2 | | 3.45 | 0.21 | 2 | 20.95 | 0.35 | 2 |

### Example 8. Base Editing with Chemically Modified sgRNA in PMH

Base editor constructs comprising an APOBEC3A deaminase domain fused to Nme2Cas9 nickase were tested for base conversion efficiency with various guide designs in primary mouse hepatocytes (PMH). PMH (In Vitro ADMET Laboratories, cat #MC 148) were thawed and plated as described in Example 1. Nme2Cas9 base editor mRNAs mRNA R, mRNA 5, and mRNA T; and uracil glycosylase inhibitor (UGI) mRNA (SEQ ID NO: 625) were prepared as described in Example 1 and paired with a series of chemically modified sgRNA targeted to mouse TTR and screened at a single dose of 128 ng of base editor mRNA. At 72 hours post-treatment, cells were lysed for NGS analysis as provided in Example 1. The mean editing of representative guides (ratio of edit types) is shown in Table 19.

TABLE 19
Mean editing percentages in PMH cells using modified gRNAs
targeting the TTR locus and an Nme2 base editor.

| | | C-to-T % | | | C-to-A/G % | | | Indel % | | |
|---|---|---|---|---|---|---|---|---|---|---|
| mRNA | Guide | Mean | SD | N | Mean | SD | N | Mean | SD | N |
| mRNA R | G021237 | 69.00 | 5.94 | 2 | 3.65 | 1.63 | 2 | 9.00 | 3.39 | 2 |
| | G021249 | 47.15 | 2.33 | 2 | 1.20 | 0.14 | 2 | 1.45 | 1.34 | 2 |
| | G021321 | 7.25 | 0.92 | 2 | 0.25 | 0.21 | 2 | 91.25 | 0.92 | 2 |
| mRNA S | G021237 | 76.75 | 1.34 | 2 | 4.35 | 1.48 | 2 | 7.95 | 1.34 | 2 |
| | G021249 | 54.05 | 4.17 | 2 | 1.20 | 0.28 | 2 | 1.60 | 0.42 | 2 |
| | G021321 | 0.50 | n/a | 1 | 0.10 | n/a | 1 | 99.00 | n/a | 1 |
| mRNA T | G021237 | 73.55 | 5.44 | 2 | 5.15 | 2.47 | 2 | 12.50 | 1.70 | 2 |
| | G021249 | 53.30 | 5.52 | 2 | 1.05 | 0.21 | 2 | 2.15 | 1.63 | 2 |
| | G021321 | 7.05 | 1.20 | 2 | 0.55 | 0.21 | 2 | 90.40 | 2.97 | 2 |

n/a = SD is not applicable when only 1 replicate is reported.

### Example 9. Nme2-mRNA Studies

### Example 9.1—In Vitro Editing in Primary Mouse Hepatocytes

Messenger mRNAs encoding Nme2Cas9 ORFs with different NLS placements were assayed for editing efficiency in primary mouse hepatocytes (PMH).

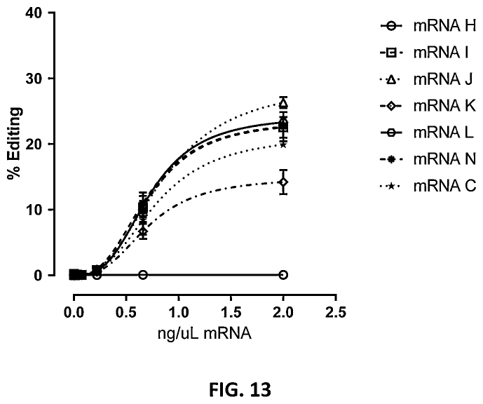

PMH were prepared as described in Example 1. Lipofection was performed using Lipofectamine MessengerMAX Transfection Reagent (Invitrogen LMRNA001) according to the manufacturer's protocol to transform cells with 100 nM sgRNA G020361 targeting mouse PCSK9 and with mRNA at the concentrations listed in Table 20. Triplicate samples were included in the assay. After 72 hours incubation at 37° C. in Maintenance Media, cells were harvested and NGS analysis was performed as described in Example 1. Mean editing results with standard deviation (SD) are shown in Table 20 and FIG. 13.

TABLE 20
Mean editing percentage in at the PCSK9 locus in PMH

| | mRNA | | |
|---|---|---|---|
| | Concentration | Mean % | |
| Construct | (ng/uL) | editing | SD |
| mRNA H | 2.00 | 0.07 | 0.05 |
| SEQ ID NO: 626 | 0.66 | 0.07 | 0.05 |
| | 0.22 | 0.03 | 0.05 |
| | 0.07 | 0.03 | 0.05 |
| | 0.03 | 0.03 | 0.05 |
| | 0.008 | 0.00 | 0.00 |
| | 0.003 | 0.00 | 0.00 |
| | 0.00 | 0.05 | 0.05 |
| mRNA I | 2.00 | 22.53 | 1.59 |
| SEQ ID NO: 627 | 0.66 | 10.37 | 2.25 |
| | 0.22 | 0.80 | 0.22 |
| | 0.07 | 0.07 | 0.05 |
| | 0.03 | 0.07 | 0.05 |
| | 0.008 | 0.03 | 0.05 |
| | 0.003 | 0.03 | 0.05 |
| | 0.00 | 0.20 | 0.28 |
| mRNA J | 2.00 | 26.30 | 0.86 |
| SEQ ID NO: 628 | 0.66 | 10.07 | 1.27 |
| | 0.22 | 0.93 | 0.33 |
| | 0.07 | 0.03 | 0.05 |
| | 0.03 | 0.03 | 0.05 |
| | 0.008 | 0.03 | 0.05 |
| | 0.003 | 0.03 | 0.05 |
| | 0.00 | 0.05 | 0.05 |
| mRNA K | 2.00 | 14.20 | 1.84 |
| SEQ ID NO: 629 | 0.66 | 6.70 | 1.16 |
| | 0.22 | 0.53 | 0.17 |
| | 0.07 | 0.07 | 0.09 |
| | 0.03 | 0.03 | 0.05 |
| | 0.008 | 0.00 | 0.00 |
| | 0.003 | 0.00 | 0.00 |
| | 0.00 | 0.05 | 0.05 |
| mRNA L | 2.00 | 23.30 | 0.80 |
| SEQ ID NO: 630 | 0.66 | 10.57 | 1.54 |
| | 0.22 | 0.70 | 0.42 |
| | 0.7 | 0.07 | 0.05 |
| | 0.03 | 0.07 | 0.05 |
| | 0.008 | 0.07 | 0.05 |
| | 0.003 | 0.03 | 0.05 |
| | 0.00 | 0.05 | 0.05 |
| mRNA N | 2.00 | 22.63 | 2.25 |
| SEQ ID NO: 631 | 0.66 | 11.00 | 0.00 |
| | 0.22 | 0.97 | 0.19 |
| | 0.07 | 0.17 | 0.09 |
| | 0.03 | 0.03 | 0.05 |
| | 0.008 | 0.03 | 0.05 |
| | 0.003 | 0.00 | 0.00 |
| | 0.00 | 0.05 | 0.05 |
| mRNA C | 2.00 | 19.90 | 0.16 |
| SEQ ID NO: 632 | 0.66 | 8.40 | 2.20 |
| | 0.22 | 0.70 | 0.22 |
| | 0.07 | 0.03 | 0.05 |
| | 0.03 | 0.00 | 0.00 |
| | 0.008 | 0.03 | 0.05 |
| | 0.003 | 0.00 | 0.00 |
| | 0.00 | 0.00 | 0.00 |

### Example 9.2—Dose Response of Nme2 ORF Variants and Guides with Chemical Modification Variations

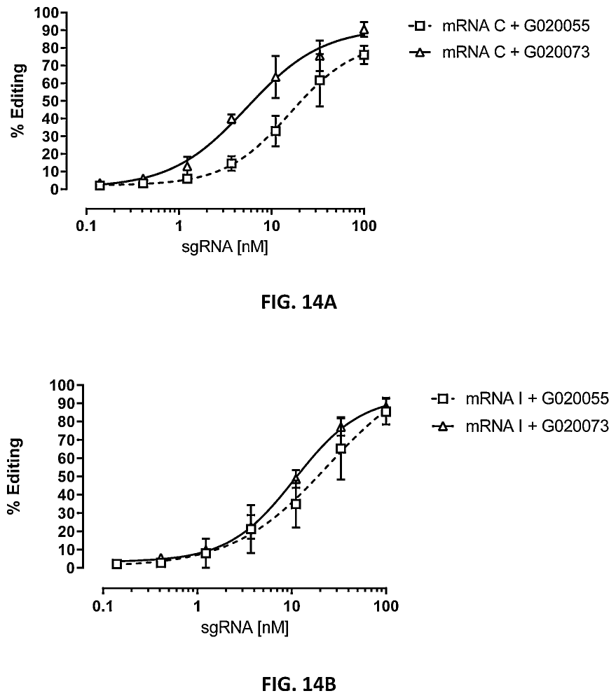

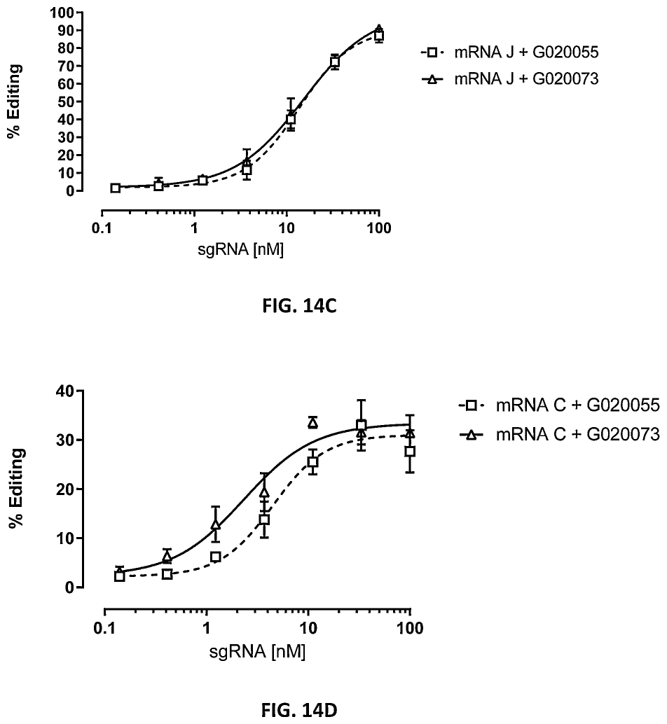

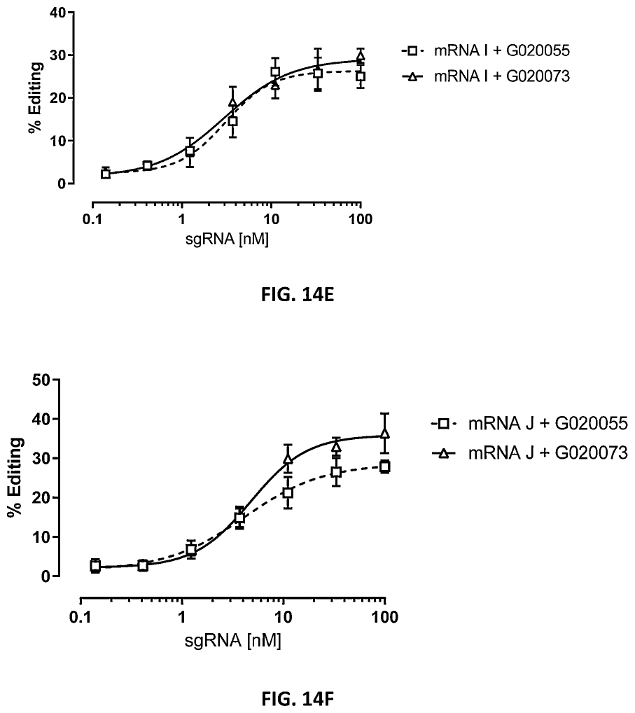

Messenger mRNAs encoding Nme2Cas9 ORFs with different NLS configurations were assayed for editing efficiency in primary human hepatocytes (PHH) and HEK-293 cells. Assays were performed using gRNAs with identical guide sequences targeting VEGFA locus TS47 and gRNAs had various lengths and chemical modification patterns. PHH cells prepared as described in Example 1. HEK293 cells were thawed and plated at a density of 30,000 cells/well in 96 well plates in DMEM (Corning, 10-013-CV) with 10% FBS and incubated for 24 hours. Lipofection was performed using Lipofectamine MessengerMAX Transfection Reagent (Invitrogen LMRNA001) according to the manufacturer's protocol. A dose response 1:3 dilution series starting at a top dose of 100 nM gRNA and 1 ng/uL mRNA, was used to transform cells with gRNA at the concentrations listed in Tables LS3.1 and LS3.2. Replicate samples were included in the assay. After 72 hours incubation at 37° C., cells were harvested and NGS analysis was performed as described in Example 1. Mean editing results with standard deviation (SD) are shown in Table 21A and FIGS. 14A-14C for HEK cells and Table 21B and FIGS. 14D-14F for PHH.

TABLE 21A
Mean percent editing in HEK cells

| | gRNA | G020055 | | | G020073 | | |
|---|---|---|---|---|---|---|---|
| mRNA | [nM] | Mean | SD | N | Mean | SD | N |
| mRNA C | 100 | 76.05 | 5.18 | 4 | 90.50 | 4.15 | 4 |
| | 33.33 | 61.68 | 14.86 | 4 | 75.55 | 8.65 | 4 |
| | 11.11 | 32.93 | 8.59 | 4 | 63.53 | 11.92 | 4 |
| | 3.70 | 14.63 | 4.08 | 4 | 39.88 | 2.40 | 4 |
| | 1.23 | 5.95 | 1.42 | 4 | 13.00 | 5.36 | 4 |
| | 0.41 | 3.35 | 0.60 | 4 | 6.03 | 1.05 | 4 |
| | 0.14 | 2.05 | 0.50 | 4 | 3.65 | 0.47 | 4 |
| | 0.00 | 1.50 | 0.22 | 4 | 1.30 | 0.22 | 4 |
| mRNA I | 100 | 85.55 | 7.06 | 4 | 88.08 | 4.90 | 4 |
| | 33.33 | 65.33 | 17.06 | 4 | 77.13 | 4.78 | 4 |
| | 11.11 | 34.98 | 12.93 | 4 | 48.63 | 4.83 | 4 |
| | 3.70 | 21.25 | 13.09 | 4 | 22.43 | 6.49 | 4 |
| | 1.23 | 8.03 | 8.06 | 4 | 9.80 | 2.12 | 4 |
| | 0.41 | 2.83 | 1.94 | 4 | 5.55 | 0.94 | 4 |
| | 0.14 | 2.15 | 0.83 | 4 | 2.45 | 0.60 | 4 |
| mRNA J | 100 | 87.03 | 3.79 | 4 | 90.93 | 1.14 | 4 |
| | 33.33 | 72.25 | 4.18 | 4 | 72.08 | 3.88 | 4 |
| | 11.11 | 40.05 | 5.01 | 4 | 42.83 | 9.02 | 4 |
| | 3.70 | 11.65 | 5.34 | 4 | 16.63 | 6.64 | 4 |
| | 1.23 | 5.78 | 0.86 | 4 | 7.00 | 1.47 | 4 |
| | 0.41 | 2.60 | 0.42 | 4 | 4.50 | 2.76 | 4 |
| | 0.14 | 1.53 | 0.50 | 4 | 1.78 | 0.24 | 4 |
| | 0.00 | 1.33 | 0.13 | 4 | 1.53 | 0.25 | 4 |

TABLE 21B
Mean percent editing in PHH cells

| | gRNA | G020055 | | | G020073 | | |
|---|---|---|---|---|---|---|---|
| mRNA | [nM] | Mean | SD | N | Mean | SD | N |
| mRNA C | 100 | 27.70 | 4.29 | 3 | 31.43 | 3.63 | 3 |
| | 33.33 | 32.98 | 5.10 | 4 | 31.58 | 2.49 | 4 |
| | 11.11 | 25.55 | 2.53 | 4 | 33.58 | 1.06 | 4 |
| | 3.70 | 13.80 | 3.68 | 4 | 19.38 | 3.86 | 4 |
| | 1.23 | 6.20 | 0.68 | 4 | 12.83 | 3.60 | 4 |
| | 0.41 | 2.70 | 0.81 | 4 | 6.35 | 1.41 | 4 |
| | 0.14 | 2.25 | 0.66 | 4 | 3.18 | 1.05 | 4 |
| | 0.00 | 1.65 | 0.24 | 4 | 1.50 | 0.18 | 4 |
| mRNA I | 100 | 25.00 | 2.73 | 4 | 29.88 | 1.67 | 4 |
| | 33.33 | 25.73 | 3.69 | 4 | 26.60 | 4.95 | 4 |
| | 11.11 | 26.08 | 3.23 | 4 | 22.98 | 3.09 | 4 |
| | 3.70 | 14.55 | 3.74 | 4 | 19.03 | 3.55 | 4 |
| | 1.23 | 7.65 | 0.70 | 4 | 7.28 | 3.41 | 4 |
| | 0.41 | 4.18 | 0.97 | 4 | 4.15 | 0.62 | 4 |
| | 0.14 | 2.18 | 0.15 | 4 | 2.83 | 0.93 | 4 |
| | 0.00 | 1.40 | 0.12 | 4 | 1.35 | 0.17 | 4 |
| mRNA J | 100 | 27.90 | 1.57 | 4 | 36.38 | 5.06 | 4 |
| | 33.33 | 26.50 | 3.59 | 4 | 32.95 | 2.27 | 4 |
| | 11.11 | 21.23 | 3.98 | 4 | 29.88 | 3.59 | 4 |
| | 3.70 | 14.85 | 2.37 | 4 | 14.88 | 2.81 | 4 |
| | 1.23 | 6.78 | 2.29 | 4 | 6.43 | 0.74 | 4 |
| | 0.41 | 2.73 | 1.35 | 4 | 3.13 | 0.49 | 4 |
| | 0.14 | 2.63 | 1.69 | 4 | 2.45 | 0.73 | 4 |
| | 0.00 | 1.40 | 0.12 | 4 | 1.50 | 0.16 | 4 |

### Example 9.3—Dose Response of Nme2 NLS Variants Using LNPs in PMH

Messenger mRNAs encoding Nme2Cas9 ORFs with different NLS placements were assayed for editing efficiency in primary mouse hepatocytes (PMH). The assay tested guides targeting the mouse TTR locus and included both sgRNA and pgRNA designs.

PMH were prepared as in Example 1. LNPs were generally prepared as described in Example 1 with a single RNA species as cargo, as indicated in Table 22. LNPs were prepared with the lipid composition of 50/9/38/3, expressed as the molar ratio of ionizable Lipid A/cholesterol/DSPC/PEG, respectively. The LNPs were formulated with a lipid amine to RNA phosphate (N:P) molar ratio of about 6.

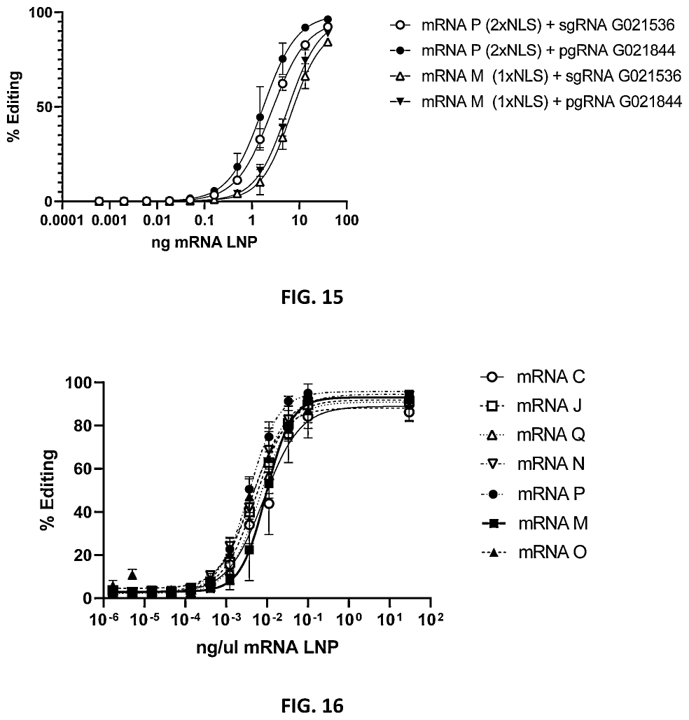

Cells were treated with 60 ng/100 μl LNP containing gRNA by RNA weight and with LNP containing mRNA as indicated in Table 22. Cells were incubated for 72 hours at 37° C. in Williams' E Medium (Gibco, A1217601) with maintenance supplements and 1000 fetal bovine serum. After 72 hours incubation at 37° C., cells were harvested and editing was assessed by NGS as described in Example 1. Mean percent editing data is shown in Table 22 and FIG. 15.

TABLE 22
Mean percent editing at the mouse TTR
locus in primary mouse hepatocytes.

| | mRNA LNP | Mean % | | |
|---|---|---|---|---|
| Sample | (ng RNA) | Editing | SD | N |
| mRNA P (2 × NLS) | 40.000 | 92.30 | 0.85 | 3 |
| G021536 | 13.330 | 82.67 | 1.61 | 3 |
| | 4.440 | 62.27 | 2.96 | 3 |
| | 1.480 | 32.80 | 4.54 | 3 |
| | 0.490 | 11.23 | 1.37 | 3 |
| | 0.160 | 3.40 | 0.71 | 3 |
| | 0.050 | 0.80 | 0.22 | 3 |
| | 0.018 | 0.30 | 0.08 | 3 |
| | 0.006 | 0.20 | 0.08 | 3 |
| | 0.002 | 0.13 | 0.05 | 3 |
| | 0.001 | 0.13 | 0.05 | 3 |
| | 0.000 | 0.10 | 0.00 | 3 |
| mRNA P (2 × NLS) | 40.000 | 96.17 | 0.12 | 3 |
| G021844 (pgRNA) | 13.330 | 91.83 | 0.34 | 3 |
| | 4.440 | 75.37 | 6.80 | 3 |
| | 1.480 | 44.53 | 13.11 | 3 |
| | 0.490 | 18.30 | 5.77 | 3 |
| | 0.160 | 5.50 | 1.43 | 3 |
| | 0.050 | 1.63 | 0.71 | 3 |
| | 0.018 | 0.33 | 0.05 | 3 |
| | 0.006 | 0.17 | 0.05 | 3 |
| | 0.002 | 0.07 | 0.05 | 3 |
| | 0.001 | 0.10 | 0.00 | 3 |
| | 0.000 | 0.07 | 0.05 | 3 |
| mRNA M (1 × NLS) | 40.000 | 84.27 | 1.23 | 3 |
| G021536 | 13.330 | 66.23 | 5.39 | 3 |
| | 4.440 | 33.80 | 5.14 | 3 |
| | 1.480 | 10.17 | 5.51 | 3 |
| | 0.490 | 4.20 | 0.92 | 3 |
| | 0.160 | 1.10 | 0.45 | 3 |
| | 0.050 | 0.33 | 0.17 | 3 |
| | 0.018 | 0.23 | 0.09 | 3 |
| | 0.006 | 0.10 | 0.00 | 3 |
| | 0.002 | 0.10 | 0.00 | 3 |
| | 0.001 | 0.10 | 0.00 | 3 |
| | 0.000 | 0.07 | 0.05 | 3 |
| mRNA M (1 × NLS) | 40.000 | 88.83 | 0.37 | 3 |
| G021844 (pgRNA) | 13.330 | 74.37 | 4.63 | 3 |
| | 4.440 | 39.00 | 3.72 | 3 |
| | 1.480 | 16.40 | 2.52 | 3 |
| | 0.490 | 4.03 | 0.77 | 3 |
| | 0.160 | 1.27 | 0.05 | 3 |
| | 0.050 | 0.23 | 0.05 | 3 |
| | 0.018 | 0.20 | 0.08 | 3 |
| | 0.006 | 0.10 | 0.00 | 3 |
| | 0.002 | 0.10 | 0.00 | 3 |
| | 0.001 | 0.10 | 0.00 | 3 |
| | 0.000 | 0.10 | 0.00 | 3 |

### Example 9.4—Dose Response of Nme2 NLS Variants Using LNPs in PMH

Messenger mRNAs encoding Nme2Cas9 ORFs with different NLS placements were assayed for editing efficiency in primary mouse hepatocytes (PMH).

PMH (Gibco, MC148) were prepared as described in Example 1. LNPs were generally prepared as described in Example 1 with a single RNA species as cargo. LNPs were prepared with the lipid composition of 50/9/38/3, expressed as the molar ratio of ionizable Lipid A/cholesterol/DSPC/PEG, respectively. The LNPs were formulated with a lipid amine to RNA phosphate (N:P) molar ratio of about 6.

Cells were treated with 30 ng by RNA weight/100 μl of LNP containing gRNA G021844 and with LNP containing mRNA as indicated in Table LS4. Cells were incubated for 24 hours in Williams' E Medium (Gibco, A1217601) with maintenance supplements and 1000 fetal bovine serum. After 72 hours incubation, cells were harvested and editing was assessed by NGS as described in Example 1. Mean percent editing data is shown in Table 23 and FIG. 16.

TABLE 23
Mean editing percentage in PMH treated with LNPs.

| | mRNA LNP | | | | EC₅₀ |
|---|---|---|---|---|---|
| mRNA | (ng/uL) | Mean | SD | N | (ng/uL) |
| mRNA C | 0.30 | 86.30 | 4.46 | 3 | 0.0082 |
| | 0.10 | 84.17 | 5.52 | | |
| | 0.03 | 75.80 | 1.91 | | |
| | 0.01 | 43.90 | 14.36 | | |
| | 0.004 | 34.03 | 8.64 | | |
| | 0.001 | 15.63 | 4.35 | | |
| | 0.0004 | 6.17 | 2.41 | | |
| | 0.0001 | 3.47 | 0.62 | | |
| | 0.00005 | 2.37 | 0.34 | | |
| | 0.00002 | 3.00 | 0.64 | | |
| | 0.00001 | 2.60 | 0.57 | | |
| | 0.00 | 2.70 | 0.16 | | |
| mRNA J | 0.30 | 91.30 | 2.92 | 3 | 0.0053 |
| | 0.10 | 89.60 | 4.23 | | |
| | 0.03 | 80.93 | 8.17 | | |
| | 0.01 | 62.85 | 14.35 | | |
| | 0.004 | 39.95 | 5.15 | | |
| | 0.001 | 16.70 | 3.79 | | |
| | 0.0004 | 7.73 | 2.98 | | |
| | 0.0001 | 4.23 | 0.95 | | |
| | 0.00005 | 2.80 | 0.70 | | |
| | 0.00002 | 3.23 | 0.54 | | |
| | 0.00001 | 2.67 | 0.48 | | |
| | 0.00 | 3.60 | 0.57 | | |
| mRNA Q | 0.30 | 90.67 | 4.40 | 3 | 0.0065 |
| | 0.10 | 86.77 | 5.43 | | |
| | 0.03 | 80.27 | 6.65 | | |
| | 0.01 | 56.90 | 5.48 | | |
| | 0.004 | 35.45 | 1.35 | | |
| | 0.001 | 12.63 | 3.16 | | |
| | 0.0004 | 5.17 | 0.56 | | |
| | 0.0001 | 2.73 | 0.17 | | |
| | 0.00005 | 2.97 | 0.41 | | |
| | 0.00002 | 2.73 | 0.21 | | |
| | 0.00001 | 2.87 | 0.56 | | |
| | 0.00 | 2.43 | 0.82 | | |
| mRNA N | 0.30 | 93.93 | 2.20 | 3 | 00045 |
| | 0.10 | 90.97 | 1.77 | | |
| | 0.03 | 82.80 | 8.24 | | |
| | 0.01 | 68.67 | 10.18 | | |
| | 0.004 | 42.07 | 2.25 | | |
| | 0.001 | 24.13 | 4.21 | | |
| | 0.0004 | 10.60 | 0.94 | | |
| | 0.0001 | 4.67 | 0.66 | | |
| | 0.00005 | 3.30 | 1.84 | | |
| | 0.00002 | 3.37 | 0.69 | | |
| | 0.00001 | 2.53 | 0.90 | | |
| | 0.00 | 2.33 | 1.48 | | |
| mRNA P | 0.30 | 94.47 | 1.04 | 3 | 0.0036 |
| | 0.10 | 95.03 | 0.96 | | |
| | 0.03 | 91.27 | 2.36 | | |
| | 0.01 | 74.77 | 6.91 | | |
| | 0.004 | 50.57 | 4.89 | | |
| | 0.001 | 22.67 | 0.25 | | |
| | 0.0004 | 8.27 | 0.74 | | |
| | 0.0001 | 4.93 | 0.70 | | |
| | 0.00005 | 3.37 | 0.74 | | |
| | 0.00002 | 2.93 | 0.68 | | |
| | 0.00001 | 2.87 | 0.05 | | |
| | 0.00 | 2.87 | 0.45 | | |
| mRNA M | 0.30 | 92.00 | 0.80 | 3 | 0.0093 |
| | 0.10 | 91.40 | 1.90 | | |
| | 0.03 | 79.70 | 0.70 | | |
| | 0.01 | 53.10 | 6.80 | | |
| | 0.004 | 22.47 | 14.28 | | |
| | 0.001 | 8.20 | 4.20 | | |
| | 0.0004 | 4.57 | 1.57 | | |
| | 0.0001 | 2.73 | 0.31 | | |
| | 0.00005 | 3.07 | 0.21 | | |
| | 0.00002 | 2.93 | 0.09 | | |
| | 0.00001 | 2.77 | 0.66 | | |
| | 0.00 | 3.47 | 1.09 | | |
| mRNA O | 0.30 | 89.40 | 7.00 | 3 | 0.0042 |
| | 0.10 | 86.83 | 12.52 | | |
| | 0.03 | 78.17 | 15.41 | | |
| | 0.01 | 64.83 | 12.48 | | |
| | 0.004 | 47.33 | 9.03 | | |
| | 0.001 | 20.67 | 7.12 | | |
| | 0.0004 | 8.60 | 2.95 | | |
| | 0.0001 | 2.47 | 1.33 | | |
| | 0.00005 | 4.13 | 0.37 | | |
| | 0.00002 | 2.80 | 0.62 | | |
| | 0.00001 | 11.13 | 231. | | |
| | 0.00 | 6.13 | 2.16 | | |

### Example 10—NmeCas9 Protein Expression

### Example 10.1 Protein Expression in Primary Human Hepatocytes

To quantify expression of each mRNA construct, mRNA and protein expression levels were measured following LNP delivery of mRNAs encoding either SpyCas9 or NmeCas9 to primary human hepatocytes.

PHH cells were prepared as described in Example 1. LNPs were generally prepared as described in Example 1 with a single RNA species as cargo. The LNPs contained Lipid A, cholesterol, DSPC, and PEG2k-DMG in a 50:38:9:3 molar ratio, respectively. The LNPs were formulated with a lipid amine to RNA phosphate (N:P) molar ratio of about 6.

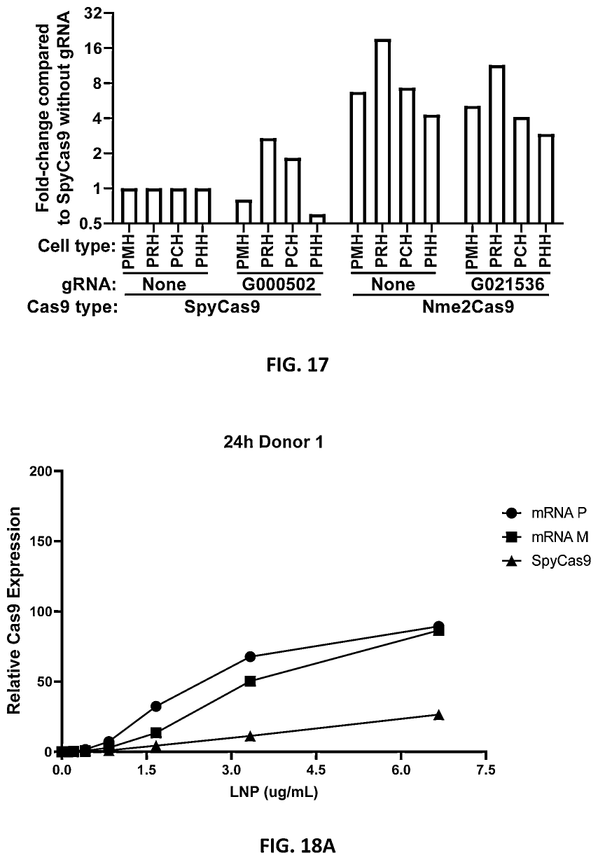

Cells were dosed with one LNP containing mRNA (mRNA only), or two LNPs containing either mRNA or gRNA. Each LNP was applied to cells at 16.7 ng total RNA cargo/100 μl. Upon treatment with LNPs, cells were incubated for 24 hours at 37° C. in Williams' E Medium (Gibco, A1217601) with maintenance supplements and 10% fetal bovine serum. After 24 hours incubation, cells were harvested and expression was quantified via Nano-Glo HiBiT lytic detection system (Promega, N3030) following manufacturer's instructions. Raw luminescence was normalized to a standard curve using HiBiT Control Protein (Promega, N3010). Protein expression of different Cas9 variants, shown in Table 24 and FIG. 17, was normalized to the expression of SpyCas9 measured in corresponding hepatocytes delivered with only the SpyCas9 mRNA. Consistent with the data shown in Table 24, protein expression from these same constructs was higher for the NmeCas9 construct than for the SpyCas9 construct when detected by western blot with an anti-HiBiT antibody from PHH cell extracts or as measured by HiBiT detection in PMH, PCH, PHH, and PRH cells.

TABLE 24
Mean fold-expression of Cas9 variants as compared to SpyCas9
expression in corresponding hepatocytes delivered with
only the SpyCas9 mRNA, as measured by the HiBiT assay

Fold-expression

| mRNA | gRNA | Cell Type | Mean | N |
|---|---|---|---|---|
| Spy Cas9 | None | PMH | 1 | 2 |
| mRNA | | PRH | 1 | 3 |
| | | PCH | 1 | 3 |
| | | PHH | 1 | 3 |
| | G000502 | PMH | 0.8 | 2 |
| | | PRH | 2.7 | 3 |
| | | PCH | 1.8 | 3 |
| | | PHH | 0.6 | 3 |
| Nme2 Cas9 | None | PMH | 6.8 | 2 |
| mRNA M | | PRH | 19.2 | 3 |
| | | PCH | 7.3 | 3 |
| | | PHH | 4.3 | 3 |
| | G021536 | PMH | 5.1 | 2 |
| | | PRH | 11.5 | 3 |
| | | PCH | 4.1 | 3 |
| | | PHH | 3.0 | 3 |

### Example 10.2: Protein Expression in T Cells

To quantify expression of each mRNA construct, protein expression levels were measured following LNP delivery of mRNAs encoding either SpyCas9 or Nme2Cas9 to T Cells.

Healthy human donor apheresis was obtained commercially (Hemacare, Cat #). T cells from two donors (W106 and W864) were isolated by negative selection using the EasySep Human T cell Isolation Kit (Stem Cell Technology, Cat. 17951) on the MultiMACS Cell24 Separator Plus instrument according to manufacturer instruction. Isolated T cells were cryopreserved in CS10 freezing media (Cryostor, Cat., 07930) for future use.

Upon thaw, T cells were cultured in complete T cell growth media composed of CTS OpTmizer Base Media (CTS OpTmizer Media (Gibco, A1048501) with 1× GlutaMAX, 10 mM HEPES buffer, 1% Penicillin/Streptomycin)) supplemented with cytokines (200 IU/ml IL2, 5 ng/ml IL7 and 5 ng/ml IL15) and 2.5% human serum (Gemini, 100-512). After overnight rest at 37° C., T cells at a density of 1e6/mL were activated with T cell TransAct Reagent (1:100 dilution, Miltenyi) and incubated in a tissue culture incubator for 48 hours.

The activated T cells were treated with LNPs delivering mRNAs encoding Nme2-mRNA or Spy mRNA with HiBiT tags. LNPs were generally prepared as in Example 1. LNPs were formulated with a lipid amine to RNA phosphate (N:P) molar ratio of about 6. The LNPs encapsulating Nme2Cas9 mRNAs used Lipid A, cholesterol, DSPC, and PEG2k-DMG in a molar ratio of 50:38:9:3 respectively. The LNP encapsulating SpyCas9 mRNA used Lipid A, cholesterol, DSPC, and PEG2k-DMG in a 50:10:38.5:1.5 molar ratio, respectively. No guide RNA was provided in this experiment.

Immediately prior to LNP treatment of T cells, LNPs were preincubated at 37° C. for 5 minutes at an LNP concentration of 13.33 ug/ml total RNA with 10 ug/mL ApoE3 (Peprotech, Cat #350-02) in complete T cell media supplemented with cytokines (200 IU/ml IL2 (Peprotech, Cat. 200-02), 5 ng/ml IL7 (Peprotech, Cat. 200-07), and 5 ng/ml IL15 (Peprotech, Cat. 200-15) and 2.5% human serum (Gemini, 100-512). After incubation, LNPs were then mixed 1:1 by volume with T cells in the complete T cell media with cytokines used for ApoE incubation. T cells were harvested for protein expression analysis at 24 h, 48 h, and 72 h post LNP treatment. T cells were lysed by Nano-Glo® HiBiT Lytic Assay (Promega) and Cas9 protein levels quantified via Nano-Glo® Nano-Glo HiBiT Extracellular Detection System (Promega, Cat. N2420) following the manufacturer's instructions. Luminescence was measured using the Biotek Neo2 plate reader. Linear regression was plotted on GraphPad using the protein number and luminescence readouts from the standard controls, forcing the line to go through X=0, Y=0. Used the Y=ax+0 equation to calculate number of proteins per lysate.

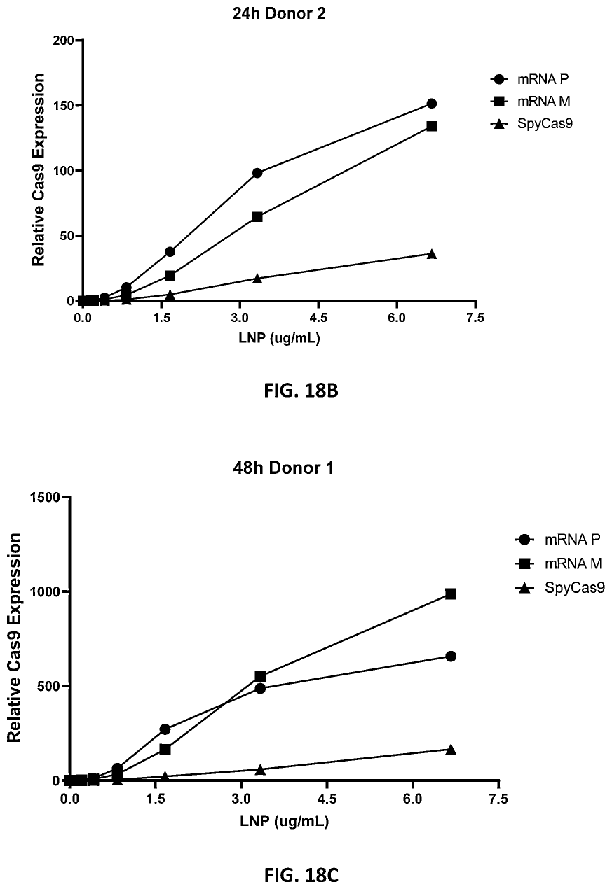

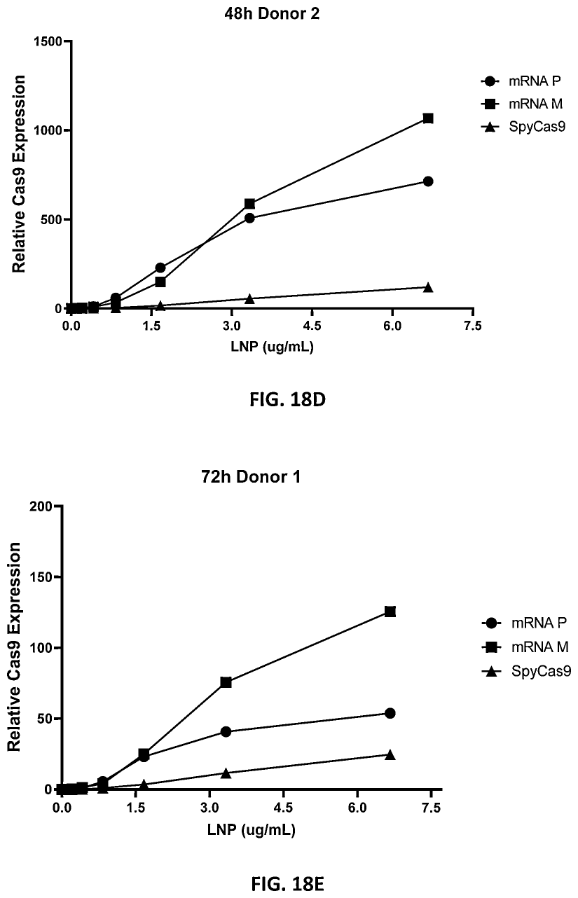

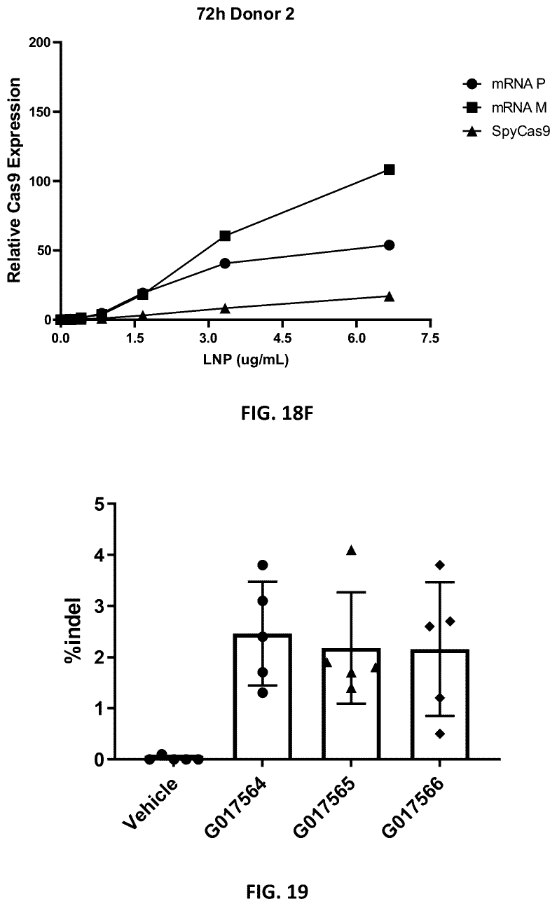

Samples were normalized to the mean of SpyCas9 at 0.83 ug/ml LNP dose. Tables 25A-25B, and FIGS. 18A-18F show the relative Cas9 protein expression in activated cells when mRNA at 24, 48, and 72 hours post LNP treatment in Donor 1 or Donor 2. Cas9 was expressed in a dose dependent manner in activated T cells. Protein expression was higher from Nme2Cas9 samples in comparison to the SpyCas9 sample in activated T cells.

TABLE 25A
Protein expression normalized to the mean
SpyCas9 0.83 ug/ml sample for donor 1

| | | T cell Donor 1 | | | | | |
|---|---|---|---|---|---|---|---|
| Timepoint | LNP | mRNA P | | mRNA M | | Spy Cas9 | |
| (hours) | (ug/mL) | Mean | N | Mean | N | Mean | N |
| 24 h | 6.67 | 89.3 | 2 | 86.5 | 2 | 26.5 | 2 |
| | 3.33 | 67.7 | 2 | 50.3 | 2 | 11.3 | 2 |
| | 1.67 | 32.4 | 2 | 13.5 | 2 | 4.4 | 2 |
| | 0.83 | 7.3 | 2 | 2.9 | 2 | 1.0 | 2 |
| | 0.42 | 1.6 | 2 | 0.8 | 2 | 0.2 | 2 |
| | 0.21 | 0.4 | 2 | 0.3 | 2 | 0.1 | 2 |
| | 0.10 | 0.2 | 2 | 0.1 | 2 | 0.0 | 2 |
| | 0.00 | 0.0 | 2 | 0.0 | 2 | 0.0 | 2 |
| 48 h | 6.67 | 657.2 | 2 | 987.0 | 2 | 165.9 | 2 |
| | 3.33 | 487.4 | 2 | 551.0 | 2 | 58.5 | 2 |
| | 1.67 | 271.0 | 2 | 165.1 | 2 | 21.4 | 2 |
| | 0.83 | 64.5 | 2 | 32.3 | 2 | 4.3 | 2 |
| | 0.42 | 11.8 | 2 | 7.8 | 2 | 1.0 | 2 |
| | 0.21 | 3.2 | 2 | 2.6 | 2 | 0.1 | 2 |
| | 0.10 | 1.1 | 2 | 0.7 | 2 | 0.1 | 2 |
| | 0.00 | 0.0 | 2 | 0.0 | 2 | 0.0 | 2 |
| 72 h | 6.67 | 53.8 | 2 | 125.6 | 2 | 24.6 | 2 |
| | 3.33 | 40.8 | 2 | 75.6 | 2 | 11.6 | 2 |
| | 1.67 | 23.1 | 2 | 25.0 | 2 | 3.5 | 2 |
| | 0.83 | 5.6 | 2 | 4.0 | 2 | 1.0 | 2 |
| | 0.42 | 1.0 | 2 | 1.3 | 2 | 0.2 | 2 |
| | 0.21 | 0.2 | 2 | 0.4 | 2 | 0.0 | 2 |
| | 0.10 | 0.2 | 2 | 0.0 | 2 | 0.1 | 2 |
| | 0.00 | 0.0 | 2 | 0.0 | 2 | 0.0 | 2 |

TABLE 25B
Protein expression normalized to the mean
SpyCas9 0.83 ug/ml sample for donor 2

| | | T cell Donor 2 | | | | | |
|---|---|---|---|---|---|---|---|
| Timepoint | LNP | mRNA P | | mRNA M | | SpyCas9 | |
| (hours) | (ug/mL) | Mean | SD | Mean | SD | Mean | SD |
| 24 h | 6.67 | 151.5 | 2 | 134.0 | 2 | 36.2 | 2 |
| | 3.33 | 98.2 | 2 | 64.5 | 2 | 17.2 | 2 |
| | 1.67 | 37.6 | 2 | 19.4 | 2 | 4.9 | 2 |
| | 0.83 | 10.4 | 2 | 4.5 | 2 | 1.0 | 2 |
| | 0.42 | 2.4 | 2 | 1.0 | 2 | 0.2 | 2 |
| | 0.21 | 0.7 | 2 | 0.4 | 2 | 0.1 | 2 |
| | 0.10 | 0.3 | 2 | 0.2 | 2 | 0.0 | 2 |
| | 0.00 | 0.0 | 2 | 0.0 | 2 | 0.0 | 2 |
| 48 h | 6.67 | 713.6 | 2 | 1067.3 | 2 | 119.0 | 2 |
| | 3.33 | 507.5 | 2 | 587.1 | 2 | 54.9 | 2 |
| | 1.67 | 229.0 | 2 | 149.2 | 2 | 15.9 | 2 |
| | 0.83 | 59.1 | 2 | 33.8 | 2 | 3.6 | 2 |
| | 0.42 | 10.5 | 2 | 8.3 | 2 | 1.0 | 2 |
| | 0.21 | 3.1 | 2 | 2.8 | 2 | 0.3 | 2 |
| | 0.10 | 1.0 | 2 | 1.9 | 2 | 0.2 | 2 |
| | 0.00 | 0.0 | 2 | 0.2 | 2 | 0.0 | 2 |
| 72 h | 6.67 | 53.8 | 2 | 108.2 | 2 | 17.1 | 2 |
| | 3.33 | 40.7 | 2 | 60.5 | 2 | 8.3 | 2 |
| | 1.67 | 19.3 | 2 | 18.2 | 2 | 3.1 | 2 |
| | 0.83 | 4.7 | 2 | 3.8 | 2 | 1.0 | 2 |
| | 0.42 | 1.3 | 2 | 1.4 | 2 | 0.3 | 2 |
| | 0.21 | 0.4 | 2 | 0.4 | 2 | 0.1 | 2 |
| | 0.10 | 0.3 | 2 | 0.0 | 2 | 0.4 | 2 |
| | 0.00 | 0.0 | 2 | 0.1 | 2 | 0.0 | 2 |

### Example 11. In Vivo Editing in Mouse Liver Using Lipid Nanoparticles (LNPs)

The LNPs used in all in vivo studies were formulated as described in Example 1. Deviations from the protocol are noted in the respective Example. Transport and storage solution (TSS) used in LNP preparation was dosed in the experiment as a vehicle-only negative control.

### In Vivo Editing in the Mouse Model

Selected guide designs were tested for editing efficiency in vivo. CD-1 female mice, ranging 6-10 weeks of age were used in each study involving mice. Animals were weighed pre-dose. LNPs were formulated generally as described in Example 1. LNPs contained ionizable Lipid A, cholesterol, DSPC, and PEG2k-DMG in a 50:38:9:3 molar ratio, respectively. The lipid nucleic acid assemblies were formulated with a lipid amine to RNA phosphate (N:P) molar ratio of about 6.

LNPs were dosed via the lateral tail vein at a volume of 0.2 mL per animal (approximately 10 mL per kilogram body weight). Body weight was measured at twenty-four hours post-administration. About 6-7 days after LNP delivery, animals were euthanized by exsanguination under isoflurane anesthesia post-dose. Blood was collected via cardiac puncture into serum separator tubes. For studies involving in vivo editing, liver tissue was collected from the left medial lobe from each animal for DNA extraction and analysis.

For the in vivo studies, genomic DNA was extracted from tissue using a bead-based extraction kit, e.g., the Zymo Quick-DNA 96 kit (Zymo Research, Cat. #D3010) according to the manufacturer's protocol. NGS analysis was performed as described in Example 1.

### Transthyretin (TTR) ELISA Analysis Used in Animal Studies

Blood was collected, and the serum was isolated as described above. The total TTR serum levels were determined using a Mouse Prealbumin (Transthyretin) ELISA Kit (Aviva Systems Biology, Cat. OKIA00111). Kit reagents and standards were prepared according to the manufacturer's protocol. Mouse serum was diluted to a final dilution of 10,000-fold with 1× assay diluent. 10,000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a Clariostar plate reader at an absorbance of 450 nm. Serum TTR levels were calculated by SoftMax Pro software ver. 6.4.2 or Mars software ver. 3.31 using a four-parameter logistic curve fit off the standard curve. Final serum values were adjusted for the assay dilution. Percent protein knockdown (% KD) values were determined relative to controls, which generally were animals sham-treated with vehicle (TSS) unless otherwise indicated. Percent TSS was calculated by division of each sample TTR value by the average value of the TSS group then adjusted to a percentage value.

### Example 11.1. In Vivo Editing Using Co-Formulated LNPs

The editing efficiency of the modified sgRNAs tested in Example D.2 were further evaluated in a mouse model. Guide RNA designs with identical guide sequences targeting mouse PCSK9 but with conserved regions differing lengths were tested LNPs were prepared as described in Example 1. The LNPs were prepared using ionizable lipid A, cholesterol, DSPC, and PEG2k-DMG in a 50:38:9:3 molar ratio, respectively. The LNPs were formulated with a lipid amine to RNA phosphate (N:P) molar ratio of about 6. A gRNA targeting the PCSK9 gene, as indicated in Table 26, and mRNA C were co-formulated at 1:2 gRNA to mRNA by weight in LNPs. LNPs were administered to female CD-1 mice (n=5) at a dose of 1 mg/kg of total RNA as described above. Mice were euthanized at 7 days post dosing. The editing efficiency for LNPs containing the indicated sgRNAs are shown in Table 26 and illustrated in FIG. 19.

TABLE 26
Mean percent editing in mouse liver.

| Guide | Dose (mg/kg) | Mean % Edit | SD |
|---|---|---|---|
| Vehicle | — | 0.0 | 0.0 |
| G017564 | 1 | 2.5 | 0.9 |
| G017565 | 1 | 2.2 | 1.0 |
| G017566 | 1 | 2.2 | 1.2 |

### Example 11.2. In Vivo Editing Using pgRNA and mRNA LNPs

The editing efficiency of modified pgRNAs were evaluated in vivo. Four nucleotides in each of the loops of the repeat/anti-repeat region, hairpin 1, and hairpin 2 were substituted with Spacer-18 PEG linkers, in addition to the guide modifications specified in the previous study in Example 11.1.

LNPs were generally prepared as described in Example 1 with a single RNA species as cargo. The LNPs contained lipid A, cholesterol, DSPC, and PEG2k-DMG in a 50:38:9:3 molar ratio, respectively. The LNPs were formulated with a lipid amine to RNA phosphate (N:P) molar ratio of about 6.

LNPs containing gRNAs targeting TTR gene indicated in Table 27 were administered to female CD-1 mice (n=5) at a dose of 0.1 mg/kg or 0.3 mg/kg of total RNA as described above. LNP containing mRNA (mRNA M; SEQ ID NO: 631) and LNP containing a pgRNA (G021846 or G021844) were delivered simultaneously at a ratio of 1:2 by RNA weight, respectively. Mice were euthanized at 7 days post dose.

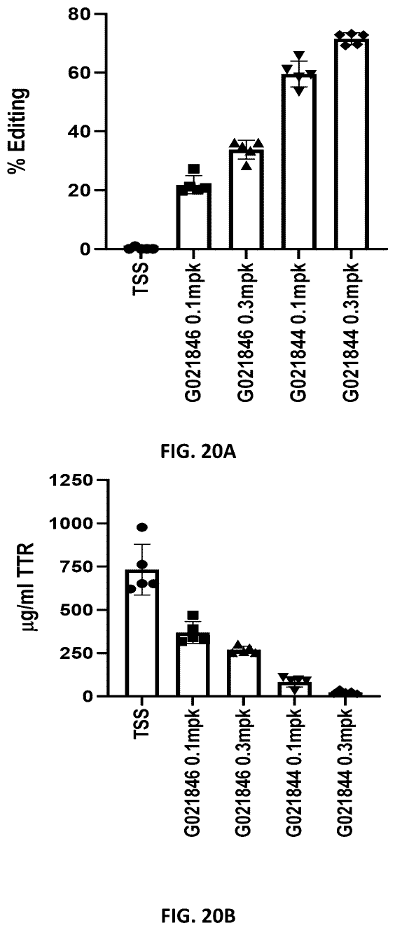

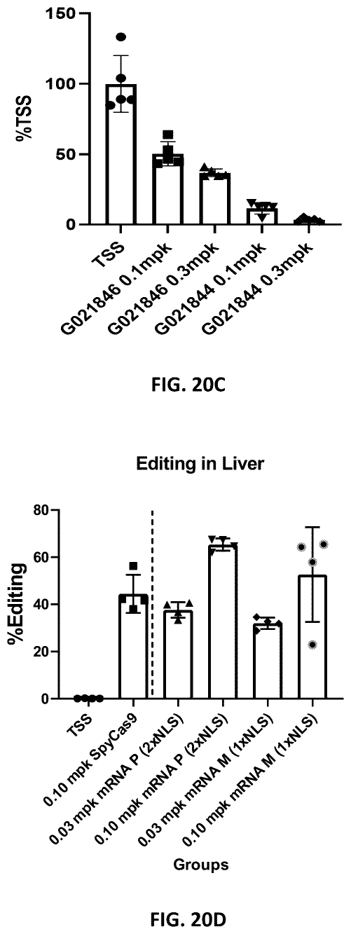

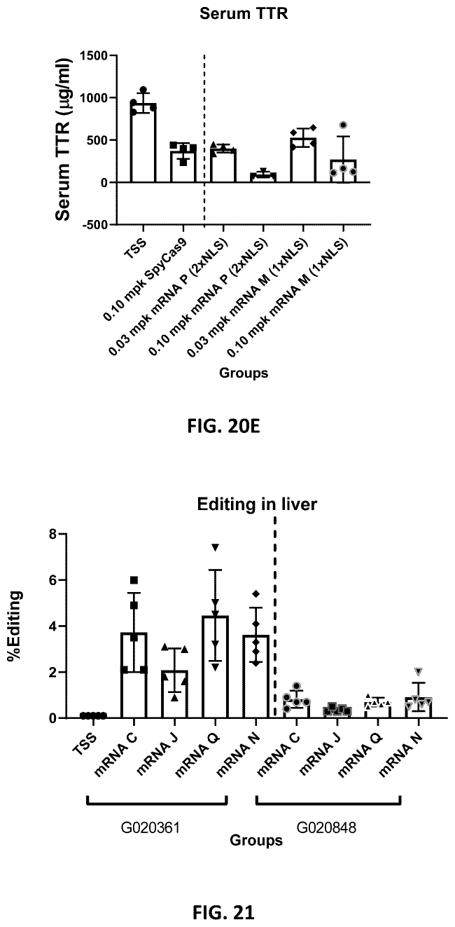

The editing efficiency, serum TTR knockdown, and percent TSS for the LNPs containing the indicated pgRNAs are shown in Table 27 and illustrated in FIGS. 20A-20C respectively.

TABLE 27
Liver Editing, Serum TTR protein, and TTR protein knockdown

| | | | | Mean | | | |
|---|---|---|---|---|---|---|---|
| | | | | serum | | | |
| | Dose | Mean % | | TTR | | Mean % | |
| Guide | (mg/kg) | Edit | SD | (ug/ml) | SD | TSS | SD |
| TSS | NA | 0.1 | 0 | 733.1 | 131.2 | 100 | 17.9 |
| G021846 | 0.1 | 21.9 | 2.8 | 369.5 | 56.2 | 50.4 | 7.7 |
| | 0.3 | 33.8 | 2.9 | 269.8 | 21.3 | 36.8 | 2.6 |
| G021844 | 0.1 | 59.6 | 3.9 | 84.1 | 26.6 | 11.5 | 3.6 |
| | 0.3 | 71.6 | 1.8 | 24.4 | 9.2 | 3.3 | 1.2 |

A pgRNA (G021844) from the study described above was evaluated in mice with alternative mRNAs at varied dose levels. LNPs were generally prepared as described in Example 1 with a single RNA species as cargo. LNPs containing pgRNA (G21844) or mRNA (mRNA P or mRNA M) were formulated as described in Example 1. The LNPs used in were prepared with ionizable lipid A, cholesterol, DSPC, and PEG2k-DMG in a 50:38:9:3 molar ratio, respectively. The LNPs were formulated with a lipid amine to RNA phosphate (N:P) molar ratio of about 6. Both G000502 and G021844 target exon 3 of the mouse TTR gene. LNP containing pgRNA and LNP containing mRNA were dosed simultaneously based on combined RNA weight at a ratio of 2:1 guide:mRNA by RNA weight, respectively. An additional LNP was co-formulated with G000502 and SpyCas9 mRNA at a ratio of 1:2 by weight, respectively, a preferred SpyCas9 guide:mRNA ratio.

LNPs indicated in Table 28 were administered to female CD-1 mice (n=4) at a dose of 0.1 mg/kg or 0.03 mg/kg of total RNA. The editing efficiency for LNPs containing the indicated gRNAs are shown in Table 28 and illustrated in FIG. 20D-20E.

TABLE 28
Liver Editing and Serum TTR protein knockdown

| | | | | | Mean | |
|---|---|---|---|---|---|---|
| | | | | | serum | |
| | | Dose | Mean % | | TTR | |
| Guide | mRNA | (mg/kg) | Edit | SD | (ug/ml) | SD |
| TSS | TSS | NA | 0.12 | 0.04 | 937.4 | 100.5 |
| G000502 | SpyCas9 | 0.1 | 44.50 | 6.9 | 370.7 | 80.1 |
| G021844 | mRNA P | 0.03 | 37.70 | 2.9 | 398.7 | 41.9 |
| | SEQ ID | 0.1 | 65.40 | 2.2 | 92.8 | 27.5 |
| | NO: 634 | | | | | |
| | mRNA M | 0.03 | 32.02 | 2.1 | 527.4 | 93.6 |
| | SEQ ID | | | | | |
| | NO: 631 | 0.1 | 62.50 | 17.4 | 268.6 | 236.8 |

### Example 11.3. In Vivo Editing Using sgRNA and mRNA LNPs

LNPs were generally prepared as described in Example 1 with a single RNA species as cargo. The LNPs used in were prepared with Lipid A, cholesterol, DSPC, and PEG2k-DMG in a 50:38:9:3 molar ratio, respectively. The LNPs were formulated with a lipid amine to RNA phosphate (N:P) molar ratio of about 6. The sgRNAs were designed to target the pcsk9 gene (G020361) or the Rosa26 gene (G020848).

LNPs containing sgRNA or mRNA were administered to female CD-1 mice (n=5) at a dose of 1 mg/kg of total RNA. The mRNAs tested (mRNA C (SEQ ID NO:622), mRNA J (SEQ ID NO: 628), mRNA Q (SEQ ID NO: 635), mRNA N (SEQ ID NO: 632) were designed with varying numbers and arrangements of NLS. LNPs were dosed simultaneously based on the combined weight of RNA cargo at a 1:1 ratio of gRNA:mRNA by RNA weight. Mean percent editing is shown in Table 29 and illustrated in FIG. 21.

TABLE 29
Mean percent editing in mouse liver.

| Guide | mRNA | Dose (mg/kg) | Mean % indel | SD |
|---|---|---|---|---|
| TSS | TSS | NA | 0.1 | 0.0 |
| G020361 | mRNA C | 1 | 3.7 | 1.5 |
| | mRNA J | 1 | 2.1 | 0.8 |
| | mRNA Q | 1 | 4.5 | 1.8 |
| | mRNA N | 1 | 3.6 | 1.0 |
| G020848 | mRNA C | 1 | 0.8 | 0.3 |
| | mRNA J | 1 | 0.4 | 0.1 |
| | mRNA Q | 1 | 0.7 | 0.2 |
| | mRNA N | 1 | 0.9 | 0.5 |

### Example 12. In Vivo Editing with NmeCas9 and Either sgRNA or pgRNA

The editing efficiency of the modified pgRNAs tested with Nme2Cas9 was tested in a mouse model. All Nine sgRNAs tested comprised the same 24nt guide sequence targeting mTTR.

LNPs were generally prepared as described in Example 1 with a single RNA species as cargo. The LNPs used in were prepared with Lipid A, cholesterol, DSPC, and PEG2k-DMG in a 50:38:9:3 molar ratio, respectively. The LNPs were formulated with a lipid amine to RNA phosphate (N:P) molar ratio of about 6. The LNPs were mixed at a ratio of 2:1 by weight of gRNA to mRNA cargo. Dose is calculated based on the combined RNA weight of gRNA and mRNA. Transport and storage solution (TSS) used in LNP preparation was dosed in the experiment as a vehicle-only negative control.

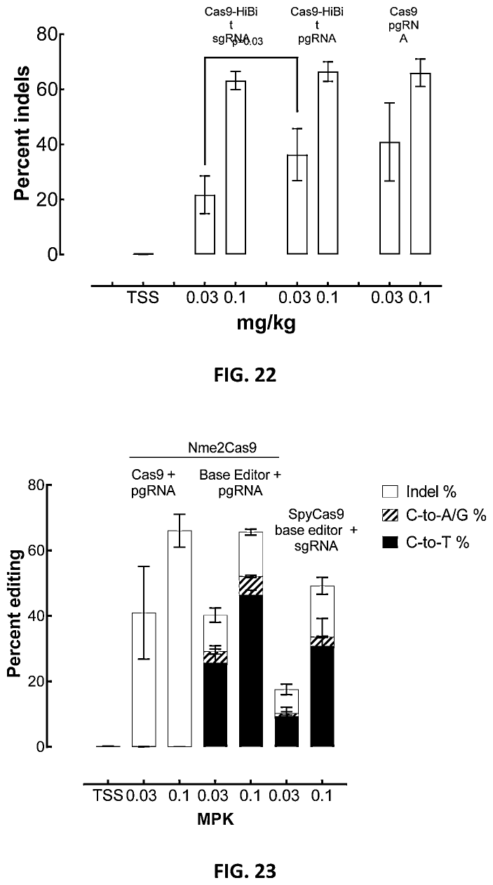

CD-1 female mice, ranging 6-10 weeks of age were used in each study involving mice (n=5 per group, except TSS control n=4). Formulations were administered intravenously via tail vein injection according to the doses listed in Table 30. Animals were periodically observed for adverse effects for at least 24 hours post-dose. Six days after treatment, animals were euthanized by cardiac puncture under isoflurane anesthesia; liver tissue was collected for downstream analysis. Liver punches weighing between 5 and 15 mg were collected for isolation of genomic DNA and total RNA. Genomic DNA samples were analyzed with NGS sequencing as described in Example 1. The editing efficiency for LNPs containing the indicated mRNAs and gRNAs are shown in Table 30 and illustrated in FIG. 22.

TABLE 30
Mean percent editing in mouse liver

| | | Dose | Mean % | | |
|---|---|---|---|---|---|
| mRNA | gRNA | (mg/kg) | Edit | SD | N |
| TSS | TSS | — | 0.08 | 0.05 | 4 |
| mRNA P | G021536 | 0.03 | 21.68 | 6.87 | 5 |
| (2 × N term | (101-nt Nme sgRNA) | 0.1 | 63.22 | 3.28 | 5 |
| NLS, HiBiT) | | | | | |
| mRNA P | G021844 | 0.03 | 36.28 | 9.45 | 5 |
| (2 × N term | (93-nt Nme pgRNA) | 0.1 | 66.44 | 3.55 | 5 |
| NLS, HiBiT) | | | | | |
| mRNA O | G021844 | 0.03 | 40.88 | 14.16 | 5 |
| (2 × N-term | (93-nt Nme pgRNA) | 0.1 | 66.02 | 5.01 | 5 |
| NLS) | | | | | |

### Example 13. In Vivo Base Editing with Nme2Cas9 gRNA

The editing efficiency of the modified gRNAs with different mRNAs were tested with Nine base editor construct in the mouse model. This experiment was performed in parallel to Example 12 and used the same control samples. LNPs were generally prepared as described in Example 1 with a single RNA species as cargo. The LNPs used were prepared with Lipid A, cholesterol, DSPC, and PEG2k-DMG in a 50:38:9:3 molar ratio, respectively. The LNPs were formulated with a lipid amine to RNA phosphate (N:P) molar ratio of about 6. The LNPs used were formulated as described in Example 1, except that each component, guide RNA, or mRNA was formulated individually into an LNP, and the LNP were mixed prior to administration as described in Table 31. For Nme2Cas9 and Nme2Cas9 base editor samples, LNPs were mixed at a ratio of 2:1 by weight of gRNA to editor mRNA cargo. For SpyCas9 base editor samples, LNPs were mixed at a ratio of 1:2 by weight of gRNA to editor mRNA cargo. Dose, as indicated in Table 31 and FIG. 14, is calculated based on the combined RNA weight of gRNA and editor mRNA. Base editor samples were treated with an additional 0.03 mpk of UGI mRNA. Transport and storage solution (TSS) used in LNP preparation was dosed in the experiment as a vehicle-only negative control.

CD-1 female mice, ranging 6-10 weeks of age were used in each study involving mice (n=5 per group, except TSS control n=4). Formulations were administered intravenously via tail vein injection according to the doses listed in Table 31. Animals were periodically observed for adverse effects for at least 24 hours post-dose. Six days after treatment, animals were euthanized by cardiac puncture under isoflurane anesthesia; liver tissue were collected for downstream analysis. Liver punches weighing between 5 and 15 mg were collected for isolation of genomic DNA and total RNA. Genomic DNA was extracted using a DNA isolation kit (ZymoResearch, D3010) and samples were analyzed with NGS sequencing as described in Example 1. The editing efficiency for LNPs containing the indicated gRNAs are shown in Table 31 and illustrated in FIG. 23.

TABLE 31
Mean percent editing in mouse liver.

| | Dose | C-to-T % | | | C-to-A/G % | | | Indel % | | |
|---|---|---|---|---|---|---|---|---|---|---|
| Sample | (mg/kg) | Mean | SD | n | Mean | SD | n | Mean | SD | n |
| TSS | 0 | 0.00 | 0.00 | 4 | 0.10 | 0.00 | 4 | 0.08 | 0.05 | 4 |
| mRNA O + G021844 | 0.03 | 0.00 | 0.00 | 5 | 0.08 | 0.04 | 5 | 40.88 | 14.16 | 5 |
| (Nme2Cas9 + pgRNA) | 0.1 | 0.00 | 0.00 | 5 | 0.02 | 0.04 | 5 | 66.02 | 5.01 | 5 |
| mRNA S + mRNA G + | 0.03 | 25.60 | 5.28 | 5 | 3.50 | 0.76 | 5 | 11.14 | 2.18 | 5 |
| G021844 (Nme2 base | 0.1 | 46.34 | 1.53 | 5 | 5.74 | 0.33 | 5 | 13.52 | 0.90 | 5 |
| editor + UGI + pgRNA) | | | | | | | | | | |
| mRNA E + mRNA G + | 0.03 | 9.28 | 2.82 | 5 | 0.94 | 0.54 | 5 | 7.34 | 1.61 | 5 |
| G000502 (SpyBC22n + | 0.1 | 30.72 | 8.51 | 5 | 2.86 | 0.23 | 5 | 15.60 | 2.58 | 5 |
| UGI + sgRNA) | | | | | | | | | | |

### Example 14. Dose Response Curve for NmeCas9 gRNA in PMH with Nme2Cas9

The editing efficiency of the modified gRNAs was tested with Nme2Cas9 construct in primary mouse hepatocytes (PMH). All Nine sgRNAs tested comprised the same 24nt guide sequence targeting the mouse TTR gene (mTTR).

PMH (Gibco, Lot MC931) were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells resuspended in hepatocyte plating medium plus supplement pack (Invitrogen, Cat. A1217601 and Gibco, Cat. CM3000). Cells were counted and plated on Bio-coat collagen I coated 96-well plates (Thermo Fisher, Cat. 877272) at a concentration of 15,000 cells/well. Plated cells were allowed to settle and adhere for 4-6 hours in a tissue culture incubator at 37° C. and 5% CO2 atmosphere. After incubation cells were checked for monolayer formation and were washed once with hepatocyte maintenance medium (Invitrogen, Cat. A1217601 and Gibco, Cat. CM4000).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of 6. Each LNP was applied to cells using an 8 point 4-fold serial dilution starting at 300 ng of total RNA per 100 μl well (about 32.25 nM gRNA concentration per well) as shown in Table 32. Upon treatment with LNPs, cells were incubated for 24 hours at 37° C. in Williams' E Medium (Gibco, A1217601) with maintenance supplements and 3% fetal bovine serum. Samples were run in triplicate. After 72 hours, cells were harvested and analyzed by NGS as described in Example 1.

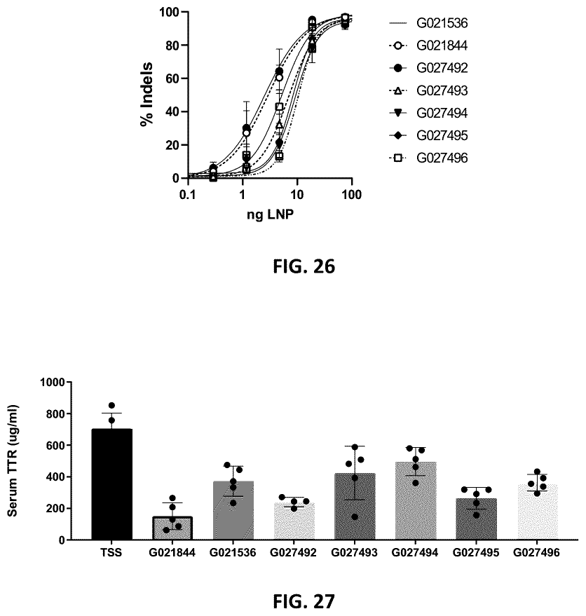

The editing efficiency for LNPs containing the indicated gRNAs, and the corresponding EC50 for each, are shown in Table 32 and illustrated in FIG. 26.

TABLE 32
Mean percent indels at the TTR locus in primary mouse hepatocytes.

| | % | ng RNA | | | | | | | | EC50 |
|---|---|---|---|---|---|---|---|---|---|---|
| Guide | indels | 300 | 75 | 18.75 | 4.68 | 1.17 | 0.29 | 0.07 | 0 | (ng RNA) |
| G021536 | Mean | 97.7 | 96.4 | 90.9 | 43.1 | 13.9 | 1.3 | 0.3 | 0.0 | 5.23 |
| | SD | 0.5 | 0.3 | 5.0 | 10.1 | 6.5 | 0.7 | 0.1 | 0.0 | |
| G021844 | Mean | 96.7 | 96.9 | 93.8 | 60.6 | 27.2 | 4.0 | 0.5 | 0.3 | 2.86 |
| | SD | 0.8 | 0.3 | 1.9 | 7.5 | 13.4 | 2.7 | 0.3 | 0.1 | |
| G027492 | Mean | 97.1 | 96.5 | 95.2 | 64.3 | 30.2 | 6.2 | 0.5 | 0.0 | 2.49 |
| | SD | 1.4 | 0.4 | 1.6 | 13.4 | 15.9 | 3.5 | 0.4 | 0.1 | |
| G027493 | Mean | 96.5 | 94.9 | 82.7 | 32.4 | 8.6 | 0.9 | 0.0 | 0.0 | 6.95 |
| | SD | 0.1 | 0.6 | 6.4 | 6.2 | 5.5 | 0.8 | 0.1 | 0.0 | |
| G027494 | Mean | 96.0 | 91.7 | 78.3 | 19.6 | 6.7 | 0.7 | 0.0 | 0.0 | 9.06 |
| | SD | 1.0 | 2.2 | 8.9 | 7.6 | 4.0 | 0.4 | 0.1 | 0.0 | |
| G027495 | Mean | 96.0 | 94.6 | 83.8 | 22.0 | 11.6 | 1.8 | 0.2 | 0.1 | 8.31 |
| | SD | 0.5 | 1.8 | 6.8 | 9.5 | 7.3 | 1.8 | 0.1 | 0.1 | |
| G027496 | Mean | 96.2 | 93.2 | 77.8 | 13.2 | 5.4 | 0.4 | 0.1 | 0.0 | 10.22 |
| | SD | 0.7 | 2.9 | 8.4 | 3.4 | 2.7 | 0.4 | 0.1 | 0.0 | |

### Example 15. In Vivo Editing with NmeCas9 gRNA

The editing efficiency of the modified gRNAs was tested with Nme2Cas9 construct in mice. All Nine sgRNAs tested comprised the same 24 nt guide sequence targeting the mouse TTR gene (mTTR).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Dose was calculated based on the combined RNA weight of gRNA and mRNA. Transport and storage solution (TSS) used in LNP preparation was dosed in the experiment as a vehicle-only negative control.

CD-1 female mice, about 6-8 weeks old, were used in each study involving mice (n=5 for all groups). Animals were fed regular chow with standard upkeep. Animals were weighed before dose administration. TSS and LNP formulations were administered intravenously via tail vein injection with a dosage of 0.03 mpk. Animals were periodically observed for adverse effects for at least 24 hours post-dose. Seven days after treatment, animals were euthanized by cardiac exsanguination under isoflurane anesthesia; blood for serum preparation and liver tissue were collected for downstream analysis.

Serum TTR levels shown in Table 33 and FIG. 27 were produced using Serum TTR ELISA—Prealbumin ELISA (Aviva Systems; cat #OKIA00111) according to the manufacturer's protocol. The level of serum TTR is significantly lower in all experimental groups compared to the negative control (TSS).

TABLE 33
Serum TTR levels (ug/ml).

| | Serum TTR | | |
|---|---|---|---|
| Guide ID | (ug/ml) | SD | % TSS |
| TSS | 704.9 | 98.3 | 100% |
| G021844 | 150.0 | 84.9 | 21% |
| G021536 | 371.1 | 95.6 | 53% |
| G027492 | 239.4 | 30.5 | 34% |
| G027493 | 423.4 | 170.0 | 60% |
| G027494 | 496.3 | 89.8 | 70% |
| G027495 | 263.6 | 68.9 | 37% |
| G027496 | 362.4 | 52.7 | 51% |

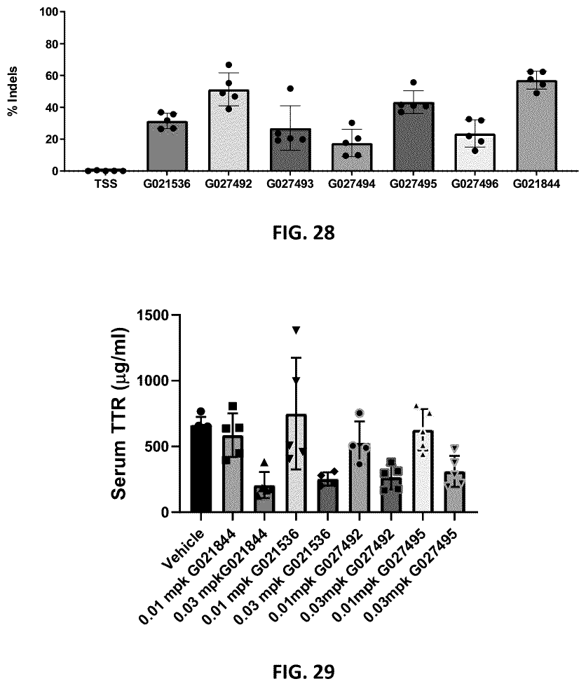

Liver biopsy punches weighing between 5 and 15 mg were collected for isolation of genomic DNA. Genomic DNA was extracted using a DNA isolation kit (ZymoResearch, D3012) and samples were analyzed with NGS sequencing (n=5 for all groups) as described in Example 1. The editing efficiency for LNPs containing the indicated gRNAs are shown in Table 34 and illustrated in FIG. 28.

TABLE 34
Mean percent indels at the TTR locus in mouse liver samples

| Guide | Mean | SD |
|---|---|---|
| TSS | 0.12 | 0.22 |
| G021844 | 57.1 | 5.7 |
| G021536 | 31.5 | 4.9 |
| G027492 | 51.3 | 10.4 |
| G027493 | 27.0 | 14.0 |
| G027494 | 17.6 | 8.6 |
| G027495 | 43.2 | 7.2 |
| G027496 | 23.5 | 8.6 |

### Example 16. In Vivo Editing with NmeCas9 gRNA

The editing efficiency of the modified gRNAs was tested with Nme2Cas9 mRNA in mice. All Nine sgRNAs tested comprised the same 24nt guide sequence targeting mTTR.

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Dose was calculated based on the combined RNA weight of gRNA and mRNA. Transport and storage solution (TSS) used in LNP preparation was dosed in the experiment as a vehicle-only negative control.

CD-1 female mice, about 6 weeks old, were used in each study involving mice (n=5 for all groups). Animals were weighed before dose administration for dose calculation, and 24 hours post-administration for monitoring. TSS and LNP formulations were administered intravenously via tail vein injection with a dosage of 0.01 mpk or 0.03 mpk. Animals were periodically observed for adverse effects for at least 24 hours post-dose. Seven days after treatment, animals were euthanized by cardiac exsanguination under isoflurane anesthesia. Blood was collected by cardiac puncture for Serum TTR ELISA, and liver tissue was collected for downstream analysis.

Serum TTR results prepared using Serum TTR ELISA—Prealbumin ELISA (Aviva Systems; cat #OKIA00111) according to the manufacturer's protocol are shown in FIG. 29 and Table 35.

TABLE 35
Serum TTR measurements following treatment.

| | Dosage | Serum TTR | | |
|---|---|---|---|---|
| Guide ID | (mpk) | (ug/ml) | SD | N |
| Vehicle | | 663.5 | 61.5 | 5 |
| G021844 | 0.01 | 585.5 | 166.1 | 5 |
| | 0.03 | 205.8 | 99.2 | 5 |
| G021536 | 0.01 | 749.2 | 425.3 | 5 |
| | 0.03 | 252.6 | 50.2 | 4 |
| G027492 | 0.01 | 527.4 | 163.1 | 4 |
| | 0.03 | 266.0 | 92.4 | 5 |
| G027495 | 0.01 | 626.9 | 157.7 | 5 |
| | 0.03 | 310.0 | 118.1 | 5 |

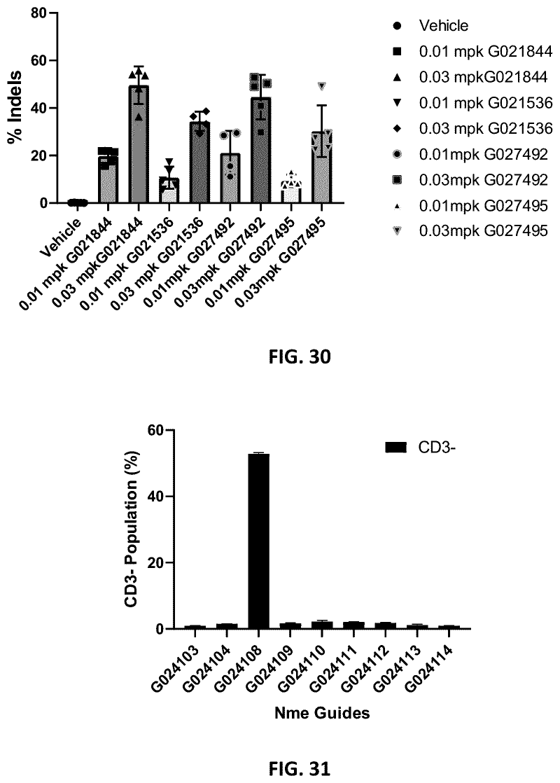

Liver biopsy punches weighing about 5 mg-15 mg were collected for isolation of genomic DNA and total RNA. Genomic DNA was extracted using a DNA isolation kit (ZymoResearch, D3012) and samples were analyzed with NGS sequencing (n=5 for all groups) as described in Example 1. The editing efficiency for LNPs containing the indicated gRNAs are shown in Table 36 and illustrated in FIG. 30.

TABLE 36
Mean percent indels at the TTR locus in mouse liver samples.

| Guide ID | Dosage | Mean | SD | N |
|---|---|---|---|---|
| Vehicle | 0.00 | 0.1 | 0.07 | 5 |
| G021844 | 0.01 | 19.7 | 2.9 | 5 |
| | 0.03 | 49.6 | 7.9 | 5 |
| G021536 | 0.01 | 10.7 | 4.7 | 5 |
| | 0.03 | 34.4 | 4.1 | 4 |
| G027492 | 0.01 | 21.1 | 9.2 | 4 |
| | 0.03 | 44.6 | 9.4 | 5 |
| G027495 | 0.01 | 9.3 | 2.6 | 4 |
| | 0.03 | 30.2 | 10.9 | 5 |

### Example 17. Guide Screen with Nme1Cas9 and Nme3Cas9 mRNAs in T Cells

The editing efficiency of one modified gRNA scaffold was tested in T cells with Nme1Cas9 or Nme3Cas9 mRNA using guides with 9 distinct target sequences in the TRAC locus.

Healthy human donor apheresis was obtained commercially (Hemacare, Donor 3786), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for use in Cryostor® CS10 (StemCell Technologies Cat. 07930). Upon thawing, T cells were plated at a density of 1.0×10\hat{ }⁶ cells/mL in T cell growth media (TCGM) composed of CTS OpTmizer T Cell Expansion SFM and T Cell Expansion Supplement (Thermo Fisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15). T cells were rested in this media for 24 hours, at which time they were activated with T Cell TransAct™, human reagent (Miltenyi, Cat. 130-111-160) added at a 1:100 ratio by volume.

For Nme1Cas9 guide screening, solutions containing mRNA encoding Nme1Cas9 (mRNA AB) were prepared in P3 buffer. Guide RNAs targeting various sites in the TRAC locus were denatured for 2 minutes at 95° C. and incubated at room temperature for 5 minutes. Forty-eight hours post activation, T cells were harvested, centrifuged, and resuspended at a concentration of 12.5×10\hat{ }⁶ cells/mL in P3 electroporation buffer (Lonza). For each well to be electroporated, 1×10\hat{ }⁵ cells were mixed with 600 ng of Nme1Cas9 mRNA and 5 μM of gRNAs in a final volume of 20 μL of P3 electroporation buffer. This mix was transferred in duplicate to 96-well Nucleofector™ plates and electroporated using the manufacturer's pulse code. Electroporated T cells were immediately rested in CTS OpTmizer T cell growth media without cytokines for 15 minutes before being transferred to new flat-bottom 96-well plates containing an additional CTS OpTmizer T cell growth media supplemented with cytokines. The resulting plates were incubated at 37° C. for 3 days. On day 3 post-electroporation, cells were split 1:2 in 2 U-bottom plates.

On day 7 post-electroporation, the plated T cells were assayed by flow cytometry to determine surface expression of the T cell receptor. Briefly, T cells were incubated with antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628). Cells were subsequently washed, resuspended in cell staining buffer and processed on a Cytoflex flow cytometer (Beckman Coulter). Flow cytometry data was analyzed using the FlowJo software package. T cells were gated based on size, shape, viability, and the expression of CD8 and CD3. Samples were run in duplicate.

The CD3 is a cell-surface component of the T cell receptor complex and its presence at the cell surface is used as a surrogate marker for TRAC protein expression. CD3 negative cell population, and corresponding standard deviation (SD) for each of the indicated gRNAs are shown in Table 37 and illustrated in FIG. 31.

TABLE 37
Mean percent CD3 negative T cells following
TRAC editing with Nme1Cas9

| Guide ID | Mean | SD |
|---|---|---|
| G024103 | 0.95 | 0.02 |
| G024104 | 1.52 | 0.04 |
| G024108 | 52.82 | 0.40 |
| G024109 | 1.69 | 0.14 |
| G024110 | 2.24 | 0.39 |
| G024111 | 2.10 | 0.06 |
| G024112 | 1.81 | 0.14 |
| G024113 | 1.19 | 0.26 |
| G024114 | 0.97 | 0.05 |

For screening of guides with Nme3Cas9 mRNA, T cells were prepared as described in this example. Solutions containing mRNA encoding Nme3Cas9 (mRNA Z) were prepared in P3 buffer, as well as controls of Nme1Cas9 (mRNA AB) and Nme2Cas9 (mRNA O). Electroporation of an NmeCas9 (e.g., Nme1Cas9, Nme2Cas9, or Nme3Cas9) gRNA and mRNA was performed as described above. Samples were electroporated in triplicate. On day 3 post electroporation, cells were assayed via flow cytometry as described above.

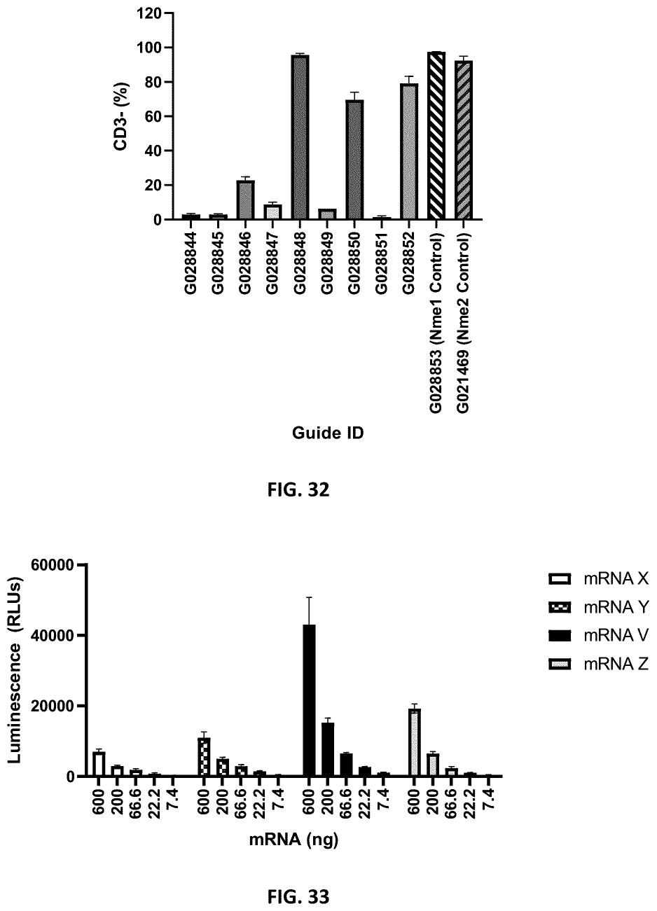

The CD3-negative cell population and corresponding standard deviation (SD) for each of the indicated gRNAs are shown in Table 38 and illustrated in FIG. 32.

TABLE 38
Mean percent CD3 negative T cells following
TRAC editing with Nme3Cas9.

| Guide ID | Mean | SD |
|---|---|---|
| G028844 | 2.99 | 0.49 |
| G028845 | 2.97 | 0.30 |
| G028846 | 22.83 | 1.65 |
| G028847 | 8.71 | 1.16 |
| G028848 | 95.6 | 0.74 |
| G028849 | 6.24 | 0.02 |
| G028850 | 69.63 | 3.57 |
| G028851 | 1.49 | 0.53 |
| G028852 | 79.13 | 3.34 |
| G028853 (Nme1 Control) | 97.43 | 0.20 |
| G021469 (Nme2 Control) | 92.46 | 2.00 |

### Example 18. Expression of Codon Optimized NmeCas9 mRNAs

To quantify expression of each mRNA construct, protein expression levels were measured following electroporation of mRNAs encoding Nme1Cas9, Nme2Cas9, or Nme3Cas9 into T cells. All of the NmeCas9 mRNA constructs have the same general structure with sequential SV40 and nucleoplasmin nuclear localization signal coding sequences N-terminal to the NmeCas9 open reading frame. Constructs include a coding sequence for a HiBiT tag C-terminal to the NmeCas9 open reading frame. The components are joined by linkers and the specific sequences are provided herein.

Healthy human donor apheresis was obtained commercially (Hemacare, Donor 3786), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930). Upon thawing, T cells were plated at a density of 1.0×10\hat{ }⁶ cells/mL in T cell growth media (TCGM) composed of CTS OpTmizer T Cell Expansion SFM and T Cell Expansion Supplement (Thermo Fisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15). T cells were rested in this media for 24 hours, at which time they were activated with T Cell TransAct™, human reagent (Miltenyi, Cat. 130-111-160) added at a 1:100 ratio by volume.

Solutions containing mRNA encoding NmeCas9 were prepared in P3 buffer. Guide RNAs targeting the TRAC locus were removed from the storage and denatured for 2 minutes at 95° C. and incubated at room temperature for 5 minutes. Forty-eight hours post activation, T cells were harvested, centrifuged, and resuspended at a concentration of 12.5×10\hat{ }⁶ cells/mL in P3 electroporation buffer (Lonza). Each well to be electroporated contained 1×10\hat{ }⁵ cells, NmeCas9 mRNA as specified in Table 39, and 1 μM gRNAs (G028853 for Nme1Cas9; G021469 for Nme2Cas9; G028848 for Nme3Cas9) as specified in Table 39 in a final volume of 20 μL of P3 electroporation buffer. NmeCas9 mRNA was tested using a three-fold, five point serial dilution starting at 600 ng mRNA. The appropriate gRNA & mRNA mix was transferred in triplicate to 96-well Nucleofector™ plates and electroporated using the manufacturer's pulse code. Electroporated T cells were immediately rested in CTS OpTmizer T cell growth media without cytokines for 15 minutes before being transferred to new flat-bottom 96-well plates containing an additional CTS OpTmizer T cell growth media supplemented with cytokines. The resulting plates were incubated at 37° C. for 24 hours prior to HiBiT luminescence assay or 96 hours prior to flow cytometry.

T cells were harvested for protein expression analysis at 24 h post-electroporation. T cells were lysed by Nano-Glo® HiBiT Lytic Assay (Promega). Luminescence was measured using the Biotek Neo2 plate reader. Table 39 and FIG. 33 show the Cas9 protein expression and corresponding standard deviation (SD) in activated cells as relative luminescence units (RLU).

TABLE 39
Mean luminescence (RLU) as a relative measure of
Cas9 protein expression in T cells at 24 hours.

| | mRNA | | | | | |
|---|---|---|---|---|---|---|
| | (ng) | 600 | 200 | 66.6 | 22.2 | 7.4 |
| mRNA X | Mean | 6955.7 | 2941.0 | 1893.7 | 758.3 | 288.7 |
| (Nme1Cas9) | (RLUs) | | | | | |
| G028853 | SD | 800.5 | 232.0 | 268.3 | 256.0 | 21.4 |
| mRNA Y | Mean | 10999.7 | 4967.6 | 2888.7 | 1423.0 | 479.3 |
| (Nme1Cas9) | (RLUs) | | | | | |
| G028853 | SD | 1621.8 | 444.6 | 451.5 | 213.3 | 42.0 |
| mRNA V | Mean | 43026.7 | 15244.0 | 6522.3 | 2658.3 | 1067.3 |
| (Nme2Cas9) | (RLUs) | | | | | |
| G021469 | SD | 7729.3 | 1288.1 | 229.0 | 174.3 | 127.1 |
| mRNA Z | Mean | 19217.3 | 6488.0 | 2386.0 | 1033.3 | 414.7 |
| (Nme3Cas9) | (RLUs) | | | | | |
| G028848 | SD | 1311.8 | 521.0 | 394.3 | 93.6 | 50.2 |

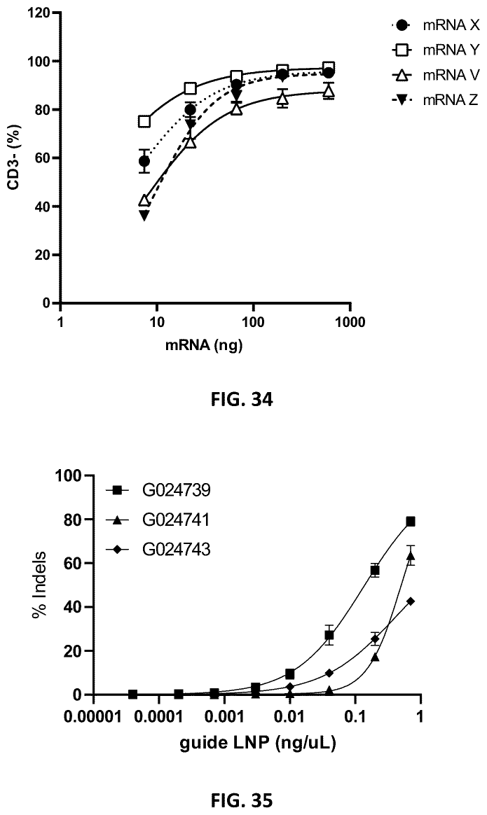

On day 4 post-editing, T cells were assayed by flow cytometry to determine surface protein expression. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100. Cells were subsequently washed, resuspended in 100 μL of cell staining buffer and processed on a Cytoflex flow cytometer (Beckman Coulter). Flow cytometry data were analyzed using the FlowJo software package. T cells were gated based on size, shape, viability, CD8, and CD3. Samples were run in triplicate. The CD3-negative cell population and corresponding standard deviation (SD) for each of the indicated gRNAs are shown in Table 40 and illustrated in FIG. 34.

TABLE 40
Percent CD3-negative cells of T cells following TRAC editing.

| | mRNA | | | | | |
|---|---|---|---|---|---|---|
| | (ng) | 600 | 200 | 66.7 | 22.2 | 7.4 |
| mRNA X | Mean | 95.2 | 94.6 | 90.3 | 79.9 | 58.7 |
| (Nme1Cas9) | SD | 0.8 | 1.1 | 0.6 | 3.1 | 4.8 |
| G028853 | | | | | | |
| mRNA Y | Mean | 97.2 | 96.2 | 93.7 | 88.7 | 75.0 |
| (Nme1Cas9) | SD | 0.8 | 1.1 | 0.9 | 1.4 | 1.3 |
| G028853 | | | | | | |
| mRNA V | Mean | 87.7 | 84.6 | 80.3 | 66.6 | 42.7 |
| (Nme2Cas9) | SD | 3.3 | 3.9 | 2.4 | 1.6 | 0.1 |
| G021469 | | | | | | |
| mRNA Z | Mean | 96.6 | 93.4 | 85.5 | 73.6 | 36.0 |
| (Nme3Cas9) | SD | 0.1 | 1.1 | 2.3 | 5.8 | 2.0 |
| G028848 | | | | | | |

### Example 19. In Vitro Editing with Selected Guides in Primary Cynomolgus Monkey Hepatocytes (PCH)

Three NmeCas9 sgRNAs (G024739, G024741, and G024743) were selected for evaluation in a dose response assay. The tested NmeCas9 sgRNAs targeting the cynomolgus TTR gene include a 24-nucleotide guide sequence.

Unmodified and modified versions of the guides are provided in Table 41.

TABLE 41
Unmodified and modified
versions of select gRNAs.

| Guide ID | Unmodified sequence | Modified sequence |
|---|---|---|
| G024739 | AGGACCAGCCUCAGACACA | mA*mG*mG*mAmCCAmGmCCm |
| | AAUACGUUGUAGCUCCCUG | UCmAGACAmCAAAmUACmGUU |
| | AAACCGUUGCUACAAUAAG | GmUmAmGmCUCCCmUmGmAmA |
| | GCCGUCGAAAGAUGUGCCG | mAmCmCGUUmGmCUAmCAAU* |
| | CAACGCUCUGCCUUCUGGC | AAGmGmCCmGmUmCmGmAmAm |
| | AUCGUU | AmGmAmUGUGCmCGmCAAmCG |
| | (SEQ ID NO: 947) | CUCUmGmCCmUmUmCmUGGCA |
| | | UCG*mU*mU |
| | | (SEQ ID NO: 928) |
| G024741 | CUGCCUCGGACGGCAUCUA | mC*mU*mG*mCmCUCmGmGAm |
| | GAACUGUUGUAGCUCCCUG | CGmGCAUCmUAGAmACUmGUU |
| | AAACCGUUGCUACAAUAAG | GmUmAmGmCUCCCmUmGmAmA |
| | GCCGUCGAAAGAUGUGCCG | mAmCmCGUUmGmCUAmCAAU* |
| | CAACGCUCUGCCUUCUGGC | AAGmGmCCmGmUmCmGmAmAm |
| | AUCGUU | AmGmAmUGUGCmCGmCAAmCG |
| | (SEQ ID NO: 948) | CUCUmGmCCmUmUmCmUGGCA |
| | | UCGumU*mU |
| | | (SEQ ID NO: 929) |

gRNAs and Cas9 mRNA were lipofected, as described below, into primary cynomolgus hepatocytes (PCH). PCH (In Vitro ADMET Laboratories 10136011) were prepared as described in Example 1. PCH were plated at a density of 40,000 cells/well. LNP formulations were prepared as described in Example 1. LNPs were prepared with the lipid composition at a molar ratio of 50% lipid A, 38% cholesterol, 9% DSPC, and 3% PEG2k-DMG. The LNPs were formulated with a lipid amine to RNA phosphate (N:P) molar ratio of about 6 and a gRNA as indicated in Table 41. PCH in 100 uL media were treated with an 8-point, 4-fold dilution series of LNP containing sgRNA, starting at 70 ng, and a fixed 30 ng dose of LNP encapsulating mRNA O by mRNA weight. The sgRNA concentration in each well is indicated in Table 42. The cells were lysed 72 hours post-treatment and NGS analysis was performed as described in Example 1. Dose response of editing efficiency to guide concentration was measured in triplicate samples. Table 42 and FIG. 35 shows mean percent editing and standard deviation (SD) at each guide concentration.

TABLE 42
Mean percent indels at TTR following editing in PCH.

| | | | |
|---|---|---|---|
| Guide LNP | G024739 | G024741 | G024743 |

| (ng/uL) | Mean | SD | Mean | SD | Mean | SD |
|---|---|---|---|---|---|---|
| 0.7 | 79.0 | 1.7 | 63.5 | 3.7 | 42.6 | 1.1 |
| 0.2 | 56.8 | 2.4 | 17.4 | 1.2 | 25.4 | 2.3 |
| 0.04 | 27.2 | 3.6 | 2.3 | 0.5 | 9.8 | 0.5 |
| 0.01 | 9.5 | 1.8 | 0.6 | 0.1 | 3.6 | 0.5 |
| 0.003 | 3.5 | 0.9 | 0.3 | 0.1 | 0.7 | 0.3 |
| 0.0007 | 0.9 | 0.3 | 0.1 | 1.3 | 0.3 | 0.1 |
| 0.0002 | 0.4 | 0.1 | 0.1 | 0.0 | 0.0 | 0.0 |
| 0.00004 | 0.2 | 0.0 | 0.1 | 1.3 | 0.0 | 0.0 |

### Example 20. In Vitro Editing of LNPs Using mRNA Dilution Series in PCH

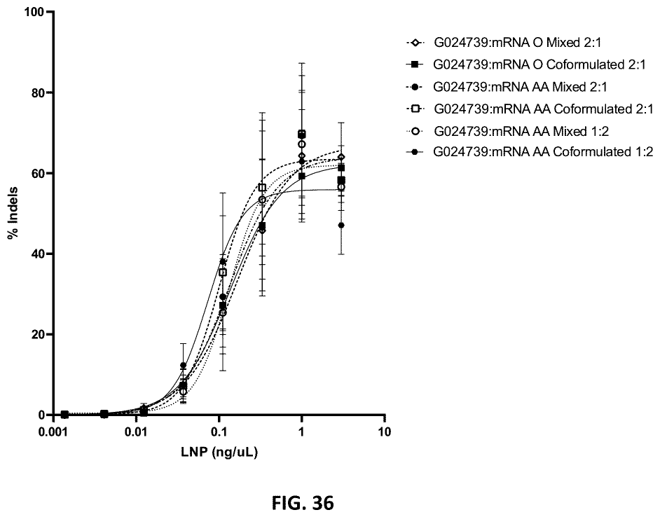

Modified sgRNAs having the same targeting site in the cynomolgus TTR gene were assayed to evaluate the editing efficiency in PCH of different mRNAs (mRNA O, mRNA AA) and formulation ratios. PCH (In Vitro ADMET Laboratories, 10136011) were prepared, treated, and analyzed as described in this example as in Example 1 unless otherwise noted. PCH were used and plated at a density of 50,000 cells/well. LNP formulations were prepared as described in Example 1. LNPs were prepared with a lipid composition having a molar ratio of 50% lipid A, 38% o cholesterol, 9% o DSPC, and 3% o PEG2k-DMG. The LNPs were formulated with a lipid amine to RNA phosphate (N:P) molar ratio of about 6 and a gRNA as indicated in Table 43. PCH in 100 uL media were treated with an 8-point, 3-fold serial dilution of mixed (separately formulated) or co-formulated LNP with various ratios of gRNA:mRNA. The top dose was 3 ng/uL total RNA by weight and gRNA:mRNA ratios for the dilution series were as indicated in Table 43. Samples were run in triplicate. Mean percent editing, standard deviation (SD), and a calculated EC50 are shown in Table 43 and in FIG. 36.

TABLE 43
Mean percent indels at the TTR locus following editing in PCH.

| | | LNP (ng/uL) | | | | | | | | EC50 |
|---|---|---|---|---|---|---|---|---|---|---|
| | | 3 | 1 | 0.33 | 0.11 | 0.04 | 0.01 | 0.004 | 0.001 | (ng/uL) |
| G024739:mRNA O | Mean % | 64 | 64.3 | 45.7 | 25.6 | 6.5 | 1.6 | 0.2 | 0.1 | 0.17 |
| LNPs Mixed 2:1 | editing | | | | | | | | | |
| | SD | 6.9 | 12.8 | 5.1 | 8.5 | 2.0 | 0.9 | 0.0 | 1.3 | |
| G024739:mRNA AA | Mean % | 58.2 | 69.2 | 46.5 | 29.3 | 7.4 | 0.6 | 0.1 | 0.0 | 0.13 |
| LNPs Mixed 2:1 | editing | | | | | | | | | |
| | SD | 4.5 | 12.2 | 13.8 | 7.6 | 3.3 | 0.3 | 0.0 | 0.0 | |
| G024739:mRNA AA | Mean % | 56.5 | 67.1 | 53.4 | 25.4 | 5.8 | 0.7 | 0.1 | 0.0 | 0.13 |
| LNPs Mixed 1:2 | editing | | | | | | | | | |
| | SD | 4.7 | 10.9 | 16.0 | 11.7 | 2.4 | 0.5 | 0.0 | 0.0 | |
| G024739:mRNA O | Mean % | 61.3 | 59.2 | 47.0 | 27.2 | 7.2 | 0.4 | 0.1 | 0.1 | 0.14 |
| Coformulated 2:1 | editing | | | | | | | | | |
| | SD | 4.4 | 9.3 | 13.2 | 8.4 | 2.2 | 0.2 | 0.0 | 0.0 | |
| G024739:mRNA AA | Mean % | 58.2 | 69.6 | 56.4 | 35.3 | 7.2 | 1.2 | 0.2 | 0.1 | 0.10 |
| Coformulated 2:1 | editing | | | | | | | | | |
| | SD | 3.0 | 14.4 | 11.4 | 11.4 | 3.3 | 0.4 | 0.1 | 1.3 | |
| G024739:mRNA AA | Mean % | 47.0 | 62.8 | 56.1 | 38 | 12.3 | 1.3 | 0.1 | 0.1 | 0.07 |
| Coformulated 1:2 | editing | | | | | | | | | |
| | SD | 5.8 | 10.5 | 15.3 | 13.9 | 4.3 | 0.1 | 0.0 | 1.3 | |

### Example 21. In Vivo Editing with NmeCas9 gRNA

The editing efficiency of the modified gRNAs was tested with Nme2Cas9 construct in mice. All Nine sgRNAs tested comprised the same 24 nt guide sequence targeting the mouse TTR gene (mTTR).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Dose was calculated based on the combined RNA weight of gRNA and mRNA. Transport and storage solution (TSS) used in LNP preparation was dosed in the experiment as a vehicle-only negative control.

CD-1 female mice, about 6-8 weeks old, were used in each study involving mice. Animals were fed regular chow with standard upkeep. Animals were weighed before dose administration. TSS and LNP formulations were administered intravenously via tail vein injection with a dosage of 0.03 mpk. Animals were periodically observed for adverse effects for at least 24 hours post-dose. Fourteen days after treatment, animals were euthanized by cardiac exsanguination under isoflurane anesthesia; blood for serum preparation and liver tissue were collected for downstream analysis.

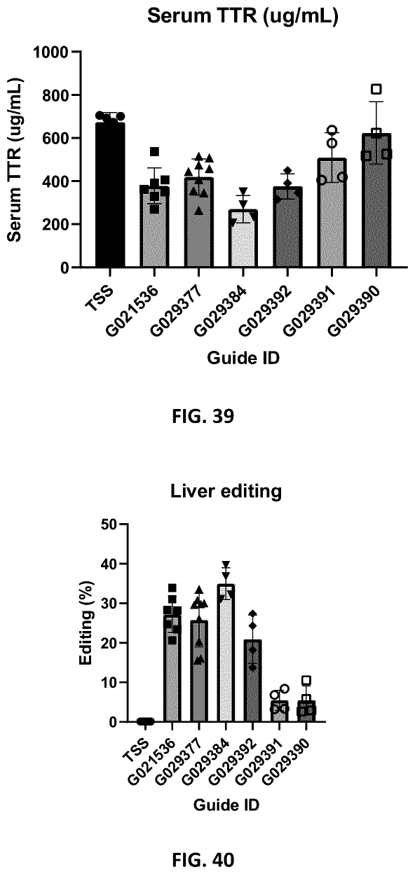

Serum TTR levels shown in Table 44 and FIG. 39 were produced using Serum TTR ELISA—Prealbumin ELISA (Aviva Systems; cat #OKIA00111) according to the manufacturer's protocol for all experimental groups and compared to the negative control (TSS).

TABLE 44
Serum TTR levels (ug/ml).

| | Serum TTR | | | |
|---|---|---|---|---|
| Guide ID | (ug/ml) | SD | % TSS | N |
| TSS | 673.7 | 44.13 | 100 | 5 |
| G021536 | 378.2 | 83.0 | 56.1 | 7 |
| G029377 | 419.3 | 83.5 | 62.2 | 9 |
| G029384 | 270.1 | 63.90 | 40.1 | 4 |
| G029392 | 375.4 | 58.23 | 55.7 | 4 |
| G029391 | 509.1 | 115.3 | 75.6 | 4 |
| G029390 | 623.2 | 144.3 | 92.5 | 4 |

Liver biopsy punches weighing between 5 and 15 mg were collected for isolation of genomic DNA. Genomic DNA was extracted using a DNA isolation kit (ZymoResearch, D3012) and samples were analyzed with NGS sequencing as described in Example 1. The editing efficiency for LNPs containing the indicated gRNAs are shown in Table 45 and illustrated in FIG. 40.

TABLE 45
Mean percent indels at the TTR locus in mouse liver samples

| Guide | Mean % editing | SD | N |
|---|---|---|---|
| TSS | 0.1 | 0 | 5 |
| G021536 | 27.2 | 4.58 | 7 |
| G029377 | 25.7 | 6.77 | 9 |
| G029384 | 34.9 | 4.05 | 4 |
| G029392 | 20.9 | 6.14 | 4 |
| G029391 | 5.4 | 2.60 | 4 |
| G029390 | 5.5 | 3.66 | 4 |

### Example 22. Dose Response Curve for NmeCas9 gRNA in PMH with Nme2Cas9

The editing efficiency of the modified gRNAs was tested with Nme2Cas9 construct in primary mouse hepatocytes (PMH). All Nine sgRNAs tested comprised the same 24nt guide sequence targeting the mouse TTR gene (mTTR).

PMH (Gibco, Lot MC931) were thawed and resuspended in hepatocyte thawing medium followed by centrifugation. The supernatant was discarded and the pelleted cells were resuspended in hepatocyte plating medium (William's E Medium (Gibco, Cat. A12176-01)) with plating supplements dexamethasone+cocktail supplement (Gibco, Cat. A15563, Lot 2459010) and FBS content (Gibco, Cat. A13450, Lot 2486425). Cells were counted and plated on Bio-coat collagen I coated 96-well plates (Corning, Ref 356407, Lot 08722018) at a concentration of 15,000 cells/well. Plated cells were allowed to settle and adhere for 4-6 hours in a tissue culture incubator at 37° C. and 5% CO2 atmosphere. After incubation cells were checked for monolayer formation and were washed once with hepatocyte maintenance medium (William's E Medium) with plating medium supplement (Gibco, Cat. A15564, Lot 2459014).

LNPs were generally prepared as described in Example 1 with a cargo of 1:2 by weight of ngRNA to mRNA. The LNPs used were prepared with a molar ratio of (500 Lipid A, 380% cholesterol, 900 DSPC, and 30% PEG2k-DMG. The LNPs were formulated with a lipid amine to RNA phosphate (N:P) molar ratio of 6. Each LNP was applied to cells using an 8 point 3-fold serial dilution starting at 450 ng of total cargo per 100 μl well at the top dose (300 ng mRNA O and 46.5 nM gRNA (about 150 ng gRNA)) as shown in Table 46. Upon treatment with LNPs, cells were incubated for 24 hours at 37° C. in William's E Medium with plating medium supplement (Gibco, Cat. A15564, Lot 2459014) and 3% o fetal bovine serum. After 72 hours, cells were harvested and analyzed by NGS as described in Example 1.

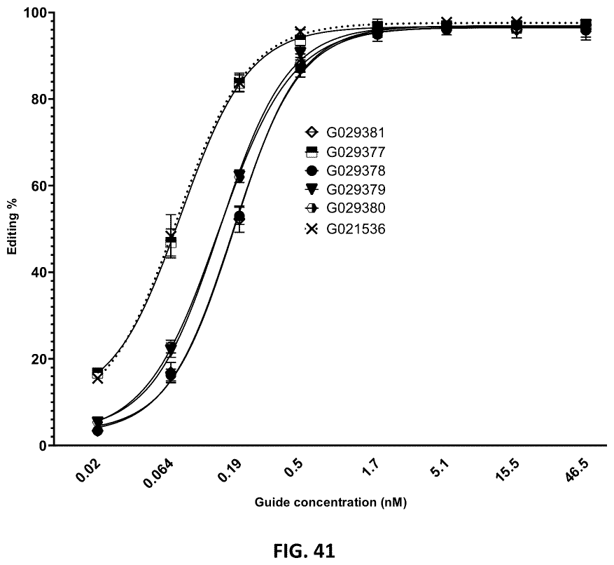

The editing efficiency for LNPs containing the indicated gRNAs, and the corresponding EC50 for each, are shown in Table 46 and illustrated in FIG. 41.

TABLE 46
Mean percent indels at the TTR locus in primary mouse hepatocytes.

| | % | nM gRNA | | | | | | | | EC50 |
|---|---|---|---|---|---|---|---|---|---|---|
| Guide | indels | 46.5 | 15.5 | 5.1 | 1.7 | 0.5 | 0.19 | 0.064 | 0.02 | (nM gRNA) |
| G021536 | Mean | 97.20 | 97.83 | 97.73 | 96.75 | 95.55 | 83.83 | 48.33 | 15.53 | 0.071 |
| | SD | 0.63 | 0.25 | 0.45 | 1.70 | 0.50 | 2.17 | 4.99 | 0.90 | |
| | N | 4.00 | 4.00 | 4.00 | 4.00 | 4.00 | 4.00 | 4.00 | 4.00 | |
| G029377 | Mean | 96.65 | 96.68 | 96.90 | 96.73 | 93.63 | 83.65 | 46.88 | 16.73 | 0.075 |
| | SD | 0.82 | 1.42 | 1.04 | 0.59 | 2.40 | 1.92 | 3.14 | 1.05 | |
| | N | 4.00 | 4.00 | 4.00 | 4.00 | 4.00 | 4.00 | 4.00 | 4.00 | |
| G029380 | Mean | 95.90 | 97.00 | 96.08 | 95.55 | 88.38 | 62.00 | 22.85 | 5.38 | 0.136 |
| | SD | 2.27 | 0.99 | 1.23 | 1.28 | 2.04 | 0.84 | 1.47 | 1.05 | |
| | N | 4.00 | 4.00 | 4.00 | 4.00 | 4.00 | 4.00 | 4.00 | 4.00 | |
| G029379 | Mean | 97.13 | 96.75 | 96.43 | 95.20 | 90.65 | 62.20 | 22.00 | 5.33 | 0.139 |
| | SD | 0.49 | 1.33 | 1.11 | 1.16 | 1.61 | 1.47 | 1.64 | 0.43 | |
| | N | 4.00 | 4.00 | 4.00 | 4.00 | 4.00 | 4.00 | 4.00 | 4.00 | |
| G029378 | Mean | 95.88 | 97.00 | 96.05 | 94.90 | 87.05 | 53.03 | 16.28 | 3.40 | 0.172 |
| | SD | 1.54 | 0.94 | 1.15 | 1.56 | 2.00 | 2.00 | 1.37 | 0.86 | |
| | N | 4.00 | 4.00 | 4.00 | 4.00 | 4.00 | 4.00 | 4.00 | 4.00 | |
| G029381 | Mean | 97.08 | 96.05 | 96.65 | 95.50 | 87.90 | 52.28 | 16.83 | 3.40 | 0.175 |
| | SD | 0.82 | 1.95 | 0.93 | 0.94 | 1.71 | 3.03 | 2.35 | 0.48 | |
| | N | 4.00 | 4.00 | 4.00 | 4.00 | 4.00 | 4.00 | 4.00 | 4.00 | |

### Example 23. Dose Response Curve for NmeCas9 gRNA in PMH with Nme2Cas9

The editing efficiency of the modified gRNAs was tested with Nme2Cas9 construct in primary mouse hepatocytes (PMH). All Nine sgRNAs tested comprised the same 24nt guide sequence targeting the mouse TTR gene (mTTR).

PMH (Gibco, Lot MC931) were thawed and resuspended in hepatocyte thawing medium followed by centrifugation. The supernatant was discarded and the pelleted cells were resuspended in hepatocyte plating medium (William's E Medium (Gibco, Cat. A12176-01)) with plating supplements dexamethasone+cocktail supplement (Gibco, Cat. A15563, Lot 2459010) and FBS content (Gibco, Cat. A13450, Lot 2486425). Cells were counted and plated on Bio-coat collagen I coated 96-well plates (Corning, Ref 356407, Lot 08722018) at a concentration of 15,000 cells/well. Plated cells were allowed to settle and adhere for 4-6 hours in a tissue culture incubator at 37° C. and 5% CO2 atmosphere. After incubation cells were checked for monolayer formation and were washed once with hepatocyte maintenance medium (William's E Medium) with plating medium supplement (Gibco, Cat. A15564, Lot 2459014).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of 6. Each LNP was applied to cells using an 8 point 3-fold serial dilution starting at 450 ng of total cargo per 100 μl well at the top dose (300 ng mRNA O and 46.5 nM gRNA (i.e., 150 ng gRNA)) as shown in Table 47. Upon treatment with LNPs, cells were incubated for 24 hours at 37° C. in William's E Medium with plating medium supplement (Gibco, Cat. A15564, Lot 2459014) and 3% fetal bovine serum. Samples were run in triplicates After 72 hours, cells were harvested and analyzed by NGS as described in Example 1.

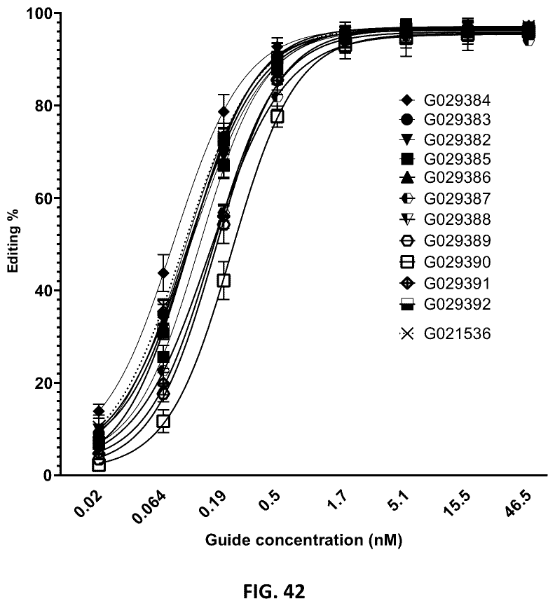

The editing efficiency for LNPs containing the indicated gRNAs, and the corresponding EC50 for each, as shown in Table 47 and illustrated in FIG. 42.

TABLE 47
Mean percent indels at the TTR locus in primary mouse hepatocytes.

| | % | nM gRNA | | | | | | | | EC50 |
|---|---|---|---|---|---|---|---|---|---|---|
| Guide | indels | 46.5 | 15.5 | 5.1 | 1.7 | 0.5 | 0.19 | 0.064 | 0.02 | (nM gRNA) |
| G029384 | Mean | 96.70 | 97.30 | 96.57 | 95.37 | 92.70 | 78.67 | 43.77 | 13.87 | 0.077 |
| | SD | 0.85 | 0.52 | 1.71 | 1.01 | 1.95 | 3.73 | 4.00 | 1.50 | |
| | N | 3.00 | 3.00 | 3.00 | 3.00 | 3.00 | 3.00 | 3.00 | 3.00 | |
| G021536 | Mean | 97.13 | 96.91 | 96.58 | 95.77 | 90.42 | 73.29 | 36.63 | 10.49 | 0.092 |
| | SD | 0.00 | 0.23 | 0.03 | 0.76 | 1.26 | 1.96 | 1.53 | 0.10 | |
| | N | 18.00 | 18.00 | 18.00 | 18.00 | 18.00 | 18.00 | 18.00 | 18.00 | |
| G029383 | Mean | 96.93 | 96.83 | 96.13 | 94.37 | 90.50 | 73.20 | 34.77 | 9.07 | 0.094 |
| | SD | 0.90 | 1.08 | 2.21 | 2.75 | 3.05 | 2.92 | 2.23 | 0.91 | |
| | N | 3.00 | 3.00 | 3.00 | 3.00 | 3.00 | 3.00 | 3.00 | 3.00 | |
| G029388 | Mean | 96.60 | 97.03 | 96.67 | 95.23 | 89.63 | 71.13 | 34.07 | 9.90 | 0.100 |
| | SD | 0.95 | 0.98 | 2.05 | 2.80 | 0.76 | 2.75 | 3.74 | 3.12 | |
| | N | 3.00 | 3.00 | 3.00 | 3.00 | 3.00 | 3.00 | 3.00 | 3.00 | |
| G029392 | Mean | 96.57 | 95.87 | 96.60 | 95.23 | 90.30 | 72.50 | 31.13 | 6.87 | 0.101 |
| | SD | 1.47 | 2.03 | 1.47 | 2.72 | 3.15 | 1.56 | 1.74 | 0.81 | |
| | N | 3.00 | 3.00 | 3.00 | 3.00 | 3.00 | 3.00 | 3.00 | 3.00 | |
| G029382 | Mean | 96.83 | 97.37 | 96.50 | 95.87 | 89.43 | 69.10 | 33.80 | 9.87 | 0.104 |
| | SD | 0.67 | 0.47 | 1.30 | 1.12 | 3.41 | 4.92 | 4.06 | 0.61 | |
| | N | 3.00 | 3.00 | 3.00 | 3.00 | 3.00 | 3.00 | 3.00 | 3.00 | |
| G029386 | Mean | 97.17 | 97.20 | 96.23 | 95.80 | 91.70 | 71.53 | 32.57 | 9.43 | 0.105 |
| | SD | 0.55 | 1.13 | 0.38 | 1.32 | 0.95 | 0.61 | 1.62 | 0.93 | |
| | N | 3.00 | 3.00 | 3.00 | 3.00 | 3.00 | 3.00 | 3.00 | 3.00 | |
| G029385 | Mean | 96.80 | 95.90 | 96.67 | 94.87 | 87.83 | 66.95 | 25.60 | 7.20 | 0.123 |
| | SD | 0.80 | 0.14 | 1.23 | 1.53 | 3.11 | 2.47 | 2.52 | 0.87 | |
| | N | 3.00 | 3.00 | 3.00 | 3.00 | 3.00 | 3.00 | 3.00 | 3.00 | |
| G029387 | Mean | 94.07 | 95.65 | 95.23 | 94.00 | 81.90 | 56.83 | 22.67 | 5.97 | 0.149 |
| | SD | 0.15 | 0.35 | 0.71 | 0.56 | 2.69 | 1.80 | 0.47 | 0.49 | |
| | N | 3.00 | 3.00 | 3.00 | 3.00 | 3.00 | 3.00 | 3.00 | 3.00 | |
| G029391 | Mean | 95.70 | 95.77 | 95.43 | 94.30 | 85.40 | 56.03 | 19.83 | 4.70 | 0.156 |
| | SD | 1.40 | 2.49 | 2.98 | 2.96 | 3.03 | 2.62 | 2.40 | 0.69 | |
| | N | 3.00 | 3.00 | 3.00 | 3.00 | 3.00 | 3.00 | 3.00 | 3.00 | |
| G029389 | Mean | 96.80 | 96.40 | 95.87 | 94.60 | 85.53 | 54.23 | 17.63 | 3.40 | 0.164 |
| | SD | 1.14 | 0.99 | 2.32 | 1.93 | 2.16 | 4.05 | 1.75 | 0.62 | |
| | N | 3.00 | 3.00 | 3.00 | 3.00 | 3.00 | 3.00 | 3.00 | 3.00 | |
| G029390 | Mean | 96.00 | 95.40 | 94.60 | 93.07 | 77.60 | 42.13 | 11.70 | 2.20 | 0.219 |
| | SD | 1.49 | 3.49 | 3.97 | 2.97 | 2.29 | 4.10 | 2.49 | 0.70 | |
| | N | 3.00 | 3.00 | 3.00 | 3.00 | 3.00 | 3.00 | 3.00 | 3.00 | |

### Example 24. Additional Embodiments

The following numbered items provide additional support for and descriptions of the embodiments herein.

Item 1 is a guide RNA (gRNA) comprising a guide region and a conserved region, the conserved region comprising one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

Item 2 is the gRNA of Item 1, wherein the gRNA is a single-guide RNA (sgRNA) and wherein the gRNA comprises (a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 500; and
(ii) nucleotide 36 is linked to nucleotide 65 by at least 2 nucleotides.

Item 3 is the gRNA of Item 1 or 2, wherein the guide region has (i) an insertion of one nucleotide or a deletion of 1-4 nucleotides within positions 1-24 relative to SEQ ID NO: 500, or (ii) a length of 24 nucleotides.

Item 4 is the gRNA of the immediately preceding Item, wherein the guide region has a length of 25, 24, 23, 22, 21, or 20 nucleotides, optionally wherein the guide region has a length of 25, 24, 23, or 22 nucleotides.

Item 5 is the gRNA of the immediately preceding Item, wherein the guide region has a length of 23-24 nucleotides.

Item 6 is the gRNA of any one of the preceding Items, wherein the gRNA further comprises a 3′ tail.

Item 7 is the gRNA of the immediately preceding Item, wherein the 3′ tail comprises 1, 2, 3, 4, 5, 6, 7, 8, 9, or 10 nucleotides.

Item 8 is the gRNA of the immediately preceding Item, wherein the 3′ tail comprises 1, 2, 3, 4, or 5 nucleotides.

Item 9 is the gRNA of any one of Items 6-8, wherein the 3′ tail terminates with a nucleotide comprising a uracil or modified uracil.

Item 10 is the gRNA of any one of Items 6-9, wherein the 3′ tail is 1 nucleotide in length.

Item 11 is the gRNA of any one of Items 6-10, wherein the 3′ tail consists of a nucleotide comprising a uracil or a modified uracil.

Item 12 is the gRNA of any one of Items 6-11, wherein the 3′ tail comprises a modification of any one or more of the nucleotides present in the 3′ tail.

Item 13 is the gRNA of any one of Items 6-12, wherein the modification of the 3′ tail is one or more of 2′-O-methyl (2′-OMe) modified nucleotide and a phosphorothioate (PS) linkage between nucleotides.

Item 14 is the gRNA of any one of the preceding Items 6-13, wherein the 3′ tail is fully modified.

Item 15 is the gRNA of any one of the preceding Items, wherein the 3′ nucleotide of the gRNA is a nucleotide comprising a uracil or a modified uracil.

Item 16 is the gRNA of any one of Items 1-5, wherein one or more of nucleotides 144 and 145 are deleted relative to SEQ ID NO: 500.

Item 17 is the gRNA of any one of Items 1-5, wherein both nucleotides 144 and 145 are deleted relative to SEQ ID NO: 500.

Item 18 is the gRNA of any one of Items 1-5, wherein the gRNA does not comprise a 3′ tail.

Item 19 is the gRNA of any one of the preceding Items, wherein the shortened repeat/anti-repeat region lacks 2-24 nucleotides.

Item 20 is the gRNA of any one of the preceding Items, wherein the shortened repeat/anti-repeat region has a length of 28, 29, 30, 31, 32, 33, 34, 35, 36, 37, 38, 39, 40, 41, 42, 43, 44, 45, 46, 47, 48, 49, or 50 nucleotides.

Item 21 is the gRNA of any one of the preceding Items, wherein the shortened repeat/anti-repeat region lacks 12-24, optionally 18-24 nucleotides, optionally 20-22 nucleotides.

Item 22 is the gRNA of any one of the preceding Items, wherein the shortened repeat/anti-repeat region has a length of 28, 29, 30, 31, 32, 33, 34, 35, 36, 37, 38, 39, or 40 nucleotides.

Item 23 is the gRNA of any one of the preceding Items, wherein the shortened repeat/anti-repeat region has a length of 28, 29, 30, 31, 32, 33, or 34 nucleotides, or 30, 31, or 32 nucleotides.

Item 24 is the gRNA of any one of the preceding Items, wherein nucleotides 37-64 of SEQ ID NO: 500 form the upper stem, and one or more base pairs of the upper stem of the shortened repeat/anti-repeat region are deleted.

Item 25 is the gRNA of any one of the preceding Items, wherein the upper stem of the shortened repeat/anti-repeat region comprises no more than one, two, three, or four base pairs.

Item 26 is the gRNA of any one of the preceding Items, wherein all of positions 39-48 and all of positions 53-62 of the upper stem of the shortened repeat/anti-repeat region are deleted, and optionally nucleotides 38 or 63 is substituted.

Item 27 is the gRNA of any one of the preceding Items, wherein all of positions 38-48 and all of positions 53-63 of the upper stem of the shortened repeat/anti-repeat region are deleted, and optionally nucleotides 37 or 64 is substituted.

Item 28 is the gRNA of any one of the preceding Items, wherein all of nucleotides 37-48 and 53-64 of the upper stem of the shortened repeat/anti-repeat region are deleted, and optionally nucleotides 36 or 65 is substituted.

Item 29 is the gRNA of any one of the preceding Items, wherein the shortened repeat/anti-repeat region has a duplex portion 11 base paired nucleotides in length.

Item 30 is the gRNA of any one of the preceding Items, wherein the shortened repeat/anti-repeat region has a single duplex portion.

Item 31 is the gRNA of any one of the preceding Items, wherein the upper stem of the shortened repeat/anti-repeat region includes one or more substitutions relative to SEQ ID NO: 500.

Item 32 is the gRNA of any one of the preceding Items, wherein one or more of nucleotides 49-52 is substituted relative to SEQ ID NO: 500.

Item 33 is the gRNA of any one of the preceding Items, wherein the shortened repeat/anti-repeat region is unsubstituted.

Item 34 is the gRNA of any one of the preceding Items, wherein the shortened repeat/anti-repeat region has 12-22 modified nucleotides

Item 35 is the gRNA of any one of the preceding Items, wherein the shortened hairpin 1 region lacks 2-10 nucleotides, optionally 2-8 or 2-4 nucleotides.

Item 36 is the gRNA of any one of the preceding Items, wherein the shortened hairpin 1 region has a length of 13, 14, 15, 16, 17, 18, 19, 20, or 21 nucleotides.

Item 37 is the gRNA of Item any one of the preceding Items, wherein the shortened hairpin 1 region has a duplex portion 4-8, optionally 7-8 base paired nucleotides in length.

Item 38 is the gRNA of Item any one of the preceding Items, wherein the shortened hairpin 1 region has a single duplex portion.

Item 39 is the gRNA of any one of the preceding Items, wherein one or two base pairs of the shortened hairpin 1 region are deleted.

Item 40 is the gRNA of any one of the preceding Items, wherein the stem of the shortened hairpin 1 region is seven or eight base paired nucleotides in length.

Item 41 is the gRNA of any one of the preceding Items, wherein one or more of positions 85-86 and one or more of nucleotides 91-92 of the shortened hairpin 1 region are deleted.

Item 42 is the gRNA of any one of the preceding Items, wherein nucleotides 86 and 91 or nucleotides 85 and 92 of the shortened hairpin 1 region are deleted.

Item 43 is the gRNA of any one of the preceding Items, wherein one or more of nucleotides 82-95 of the shortened hairpin 1 region is substituted relative to SEQ ID NO: 500.

Item 44 is the gRNA of any one of the preceding Items, wherein one or more of nucleotides 87-90 is substituted relative to SEQ ID NO: 500.

Item 45 is the gRNA of any one of the preceding Items, wherein the shortened hairpin 1 region is unsubstituted.

Item 46 is the gRNA of Item any one of the preceding Items, wherein the shortened hairpin 1 region has 6-15 modified nucleotides.

Item 47 is the gRNA of any one of the preceding Items, wherein the shortened hairpin 2 region lacks 2-18, optionally 2-16 nucleotides.

Item 48 is the gRNA of any one of the preceding Items, wherein the shortened hairpin 2 region has a length of 24, 25, 26, 27, 28, 29, 30, 31, 32, 33, 34, 35, 36, 37, 38, 39, or 40 nucleotides.

Item 49 is the gRNA of any one of the preceding Items, wherein the shortened hairpin 2 region has a length of 28, 29, 30, 31, 32, 33, or 34 nucleotides.

Item 50 is the gRNA of any one of the preceding Items, wherein one or more of nucleotides 113-121 and one or more of nucleotides 126-134 of the shortened hairpin 2 region are deleted.

Item 51 is the gRNA of any one of the preceding Items, wherein the shortened hairpin 2 region comprises an unpaired region.

Item 52 is the gRNA of any one of the preceding Items, wherein the shortened hairpin 2 region has two duplex portions.

Item 53 is the gRNA of any one of the preceding Items, wherein the shortened hairpin 2 region has a duplex portion of 4 base paired nucleotides in length.

Item 54 is the gRNA of any one of Items 52-53, wherein the shortened hairpin 2 region has a duplex portion of 4-8 base paired nucleotides in length.

Item 55 is the gRNA of any one of Items 52-54, wherein the shortened hairpin 2 region has a duplex portion of 4-6 base paired nucleotides in length.

Item 56 is the gRNA of any one of the preceding Items, wherein nucleotides 113-134 of SEQ ID NO: 500 form the upper stem, and the upper stem of the shortened hairpin 2 region comprises one, two, three, or four base pairs.

Item 57 is the gRNA of any one of the preceding Items, wherein at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted.

Item 58 is the gRNA of any one of the preceding Items, wherein all of positions 113-121 and 126-134 of the shortened hairpin 2 region are deleted.

Item 59 is the gRNA of any one of the preceding Items, wherein one or more of nucleotides 113-134 of the shortened hairpin 2 region is substituted relative to SEQ ID NO: 500.

Item 60 is the gRNA of any one of the preceding Items, wherein one or more of nucleotides 122-125 is substituted relative to SEQ ID NO: 500.

Item 61 is the gRNA of any one of the preceding Items, wherein the shortened hairpin 2 region is unsubstituted.

Item 62 is the gRNA of Item any one of the preceding Items, wherein the shortened hairpin 2 region has 6-15 modified nucleotides.

Item 63 is the gRNA of any one of the preceding Items, wherein the guide region of the gRNA comprises at least two modified nucleotides, optionally at least four modified nucleotides.

Item 64 is the gRNA of any one of the preceding Items, wherein the guide region of the gRNA comprises 1, 2, 3, 4, 5, 6, 7, 8, 9, 10, 11, or 12 modified nucleotides, optionally 1, 2, or 3 modified nucleotides.

Item 65 is the gRNA of any one of the preceding Items, wherein the guide region of the gRNA comprises 4, 5, 6, 7, 8, 9, 10, 11, or 12 modified nucleotides.

Item 66 is the gRNA of any one of the preceding Items, wherein the guide region of the gRNA comprises 6, 7, 8, 9, 10, 11, or 12 modified nucleotides.

Item 67 is the gRNA of any one of the preceding Items, wherein the guide region does not comprise a modified nucleotide 3′ of the first three nucleotides of the guide region.

Item 68 is the gRNA of any one of the preceding Items, wherein the guide region does not comprise a modified nucleotide.

Item 69 is the gRNA of any one of the preceding Items, wherein the gRNA comprises a 5′ end modification.

Item 70 is the gRNA of any one of the preceding Items, wherein the gRNA comprises a 3′ end modification.

Item 71 is the gRNA of any one of the preceding Items, wherein the gRNA comprises a 5′ end modification and a 3′ end modification.

Item 72 is the gRNA of any one of the preceding Items, comprising a modification in the upper stem region of the repeat/anti-repeat region.

Item 73 is the gRNA of any one of the preceding Items, comprising a modification in the hairpin 1 region.

Item 74 is the gRNA of any one of the preceding Items, comprising a modification in the hairpin 2 region.

Item 75 is the gRNA of any one of the preceding Items, comprising a 3′ end modification, and comprising a modification in the upper stem region of the repeat/anti-repeat region.

Item 76 is the gRNA of any one of the preceding Items, comprising a 3′ end modification, and a modification in the hairpin 1 region.

Item 77 is the gRNA of any one of the preceding Items, comprising a 3′ end modification, and a modification in the hairpin 2 region.

Item 78 is the gRNA of any one of the preceding Items, comprising a 5′ end modification, and comprising a modification in the upper stem region of the repeat/anti-repeat region.

Item 79 is the gRNA of any one of the preceding Items, comprising a 5′ end modification, and a modification in the hairpin 1 region.

Item 80 is the gRNA of any one of the preceding Items, comprising a 5′ end modification, and a modification in the hairpin 2 region.

Item 81 is the gRNA of any one of the preceding Items, comprising a 5′ end modification, a modification in the upper stem region of the repeat/anti-repeat region, and a 3′ end modification.

Item 82 is the gRNA of any one of the preceding Items, comprising a 5′ end modification, a modification in the hairpin 1 region, and a 3′ end modification.

Item 83 is the gRNA of any one of the preceding Items, comprising a 5′ end modification, a modification in the hairpin 1 region, a modification in the hairpin 2 region, and a 3′ end modification.

Item 84 is the gRNA of any one of the preceding Items, comprising a 5′ end modification, a modification in the repeat/anti-repeat region, a modification in the hairpin 1 region, a modification in the hairpin 2 region, and a 3′ end modification.

Item 85 is the gRNA of any one of Items 69-84, wherein the 5′ end modification comprises a modified nucleotide selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

Item 86 is the gRNA of any one of the Items 69-85, wherein the 3′ end modification comprises a modified nucleotide selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

Item 87 is the gRNA of any one of the Items 69-86, wherein the 5′ end modification comprises any of:
i. a modification of any one or more of the first 1, 2, 3, or 4 nucleotides;
ii. one modified nucleotide;
iii. two modified nucleotides;
iv. three modified nucleotides; and
v. four modified nucleotides.

Item 88 is the gRNA of any one of Items 69-87, wherein the 5′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide; and
v. an inverted abasic modified nucleotide.

Item 89 is the gRNA of any one of Items 69-88, wherein the 3′ end modification comprises any of:
i. a modification of any one or more of the last 4, 3, 2, or 1 nucleotides;
ii. one modified nucleotide;
iii. two modified nucleotides;
iv. three modified nucleotides; and
v. four modified nucleotides.

Item 90 is the gRNA of any one of Items 69-89, wherein the 3′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide; and
v. an inverted abasic modified nucleotide.

Item 91 is the gRNA of any one of Items 69-90, wherein the 5′ end modification comprises at least one PS linkage, and wherein one or more of:
i. there is one PS linkage, and the linkage is between the first and second nucleotides;
ii. there are two PS linkages between the first three nucleotides;
iii. there are PS linkages between any one or more of the first four nucleotides; and
iv. there are PS linkages between any one or more of the first five nucleotides.

Item 92 is the gRNA of Item 91, wherein the 5′ end modification further comprises at least one 2′-OMe, 2′-O-moe, inverted abasic, or 2′-F modified nucleotide.

Item 93 is the gRNA of any one of the preceding Items, wherein the 5′ end modification comprises:
i. a modification of one or more of the first 1-4 nucleotides, wherein the modification is a PS linkage, inverted abasic nucleotide, 2′-OMe, 2′-O-moe, or 2′-F;
ii. a modification to the first nucleotide with 2′-OMe, 2′-O-moe, or 2′-F, and an optional one or two PS linkages to the next nucleotide or the first nucleotide of the 3′ tail;
iii. a modification to the first or second nucleotide with 2′-OMe, 2′-O-moe, or 2′-F, and optionally one or more PS linkages;
iv. a modification to the first, second, or third nucleotides with 2′-OMe, 2′-O-moe, or 2′-F, and optionally one or more PS linkages; or
v. a modification to the first, second, third, or forth nucleotides with 2′-OMe, 2′-O-moe, or 2′-F, and optionally one or more PS linkages.

Item 94 is the gRNA of any one of the preceding Items, wherein the 3′ end modification comprises at least one PS linkage, and wherein one or more of:
i. there is one PS linkage, and the linkage is between the last and second to last nucleotides;
ii. there are two PS linkages between the last three nucleotides; and
iii. there are PS linkages between any one or more of the last four nucleotides.

Item 95 is the gRNA of Item 94, wherein the 3′ end modification further comprises at least one 2′-OMe, 2′-O-moe, inverted abasic, or 2′-F modified nucleotide.

Item 96 is the gRNA of any one of the preceding Items, wherein the 3′ end modification comprises:
i. a modification of one or more of the last 1-4 nucleotides, wherein the modification is a PS linkage, inverted abasic nucleotide, 2′-OMe, 2′-O-moe, or 2′-F;
ii. a modification to the last nucleotide with 2′-OMe, 2′-O-moe, or 2′-F, and an optional one or two PS linkages to the next nucleotide or the first nucleotide of the 3′ tail;
iii. a modification to the last or second to last nucleotide with 2′-OMe, 2′-O-moe, or 2′-F, and optionally one or more PS linkages;
iv. a modification to the last, second to last, or third to last nucleotides with 2′-OMe, 2′-O-moe, or 2′-F, and optionally one or more PS linkages; or
v. a modification to the last, second to last, third to last, or fourth to last nucleotides with 2′-OMe, 2′-O-moe, or 2′-F, and optionally one or more PS linkages.

Item 97 is the gRNA of any one of the preceding Items, wherein the modification in the repeat/anti-repeat region, the hairpin 1 region, or the hairpin 2 region comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, or a phosphorothioate (PS) linkage between nucleotides.

Item 98 is the gRNA of any one of the preceding Items, wherein the modification in the repeat/anti-repeat region, the hairpin 1 region, or the hairpin 2 region comprises a modified nucleotide selected from 2′-O-methyl (2′-OMe) modified nucleotide, a 2′-fluoro (2′-F) modified nucleotide, or a phosphorothioate (PS) linkage between nucleotides.

Item 99 is the gRNA of any one of the preceding Items, wherein the modification in the repeat/anti-repeat region, the hairpin 1 region, or the hairpin 2 region comprises a modified nucleotide selected from 2′-O-methyl (2′-OMe) modified nucleotide or a phosphorothioate (PS) linkage between nucleotides.

Item 100 is the gRNA of any one of the preceding Items, wherein at least 20%, 30%, 40%, or 50% of the nucleotides are modified nucleotides.

Item 101 is the gRNA of Item 100, wherein the gRNA comprises modified nucleotides selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or combinations thereof.

Item 102 is the gRNA of Item 100 or 101 comprises modified nucleotides selected from 2′-O-methyl (2′-OMe) modified nucleotide, a 2′-fluoro (2′-F) modified nucleotide, a phosphorothioate (PS) linkage between nucleotides, or combinations thereof.

Item 103 is the gRNA of any one of the preceding Items, wherein nucleotides 1-3 of the guide region are modified and nucleotides in the guide region other than nucleotides 1-3 are not modified.

Item 104 is the gRNA of any one of the preceding Items, wherein a 3′ tail of nucleotide 144 is present in the gRNA, and comprises 2′-O-Me modified nucleotides at nucleotides 141-144 and two PS linkages between nucleotides 141-142 and 142-143 respectively.

Item 105 is the gRNA of any one of the preceding Items, wherein one or more positions of 49-52, 87-90, or 122-125 is substituted.

Item 106 is a single guide RNA (sgRNA) comprising any one of SEQ ID NOs: 1-9.

Item 107 is the gRNA of any one of the preceding Items, comprising a nucleotide sequence having at least 99, 98, 97, 96, 95, 94, 93, 92, 91, 90, 85, 80, 75, or 70% identity to the nucleotide sequence of any one of SEQ ID Nos: 1-9.

Item 108 is the gRNA of any one of the preceding Items, comprising a nucleotide sequence having at least 99, 98, 97, 96, 95, 94, 93, 92, 91, 90, 85, 80, 75, or 70% identity to the nucleotide sequence of any one of SEQ ID Nos: 1-9, wherein the modification at each nucleotide of the gRNA that corresponds to a nucleotide of the reference sequence identifier in Table 1 is identical to or equivalent to the modification shown in the reference sequence identifier in Table 2.

Item 109 is the gRNA of any one of the preceding Items, comprising a nucleotide sequence having at least 99, 98, 97, 96, 95, 94, 93, 92, 91, or 90% identity to the sequence from X to the 3′ end of the nucleotide sequence of any one of SEQ ID Nos: 1-5, 7, 8, 101-291, and 301-494 where X is the first nucleotide of the conserved region.

Item 110 is the gRNA of any one of Items 106-109, further comprising a 3′ tail comprising a 2′-O-Me modified nucleotide.

Item 111 is the gRNA of any one of the preceding Items, wherein the gRNA directs a nuclease to a target sequence for binding.

Item 112 is the gRNA of any one of the preceding Items, wherein the gRNA directs a nuclease to a target sequence for inducing a double-strand break within the target sequence.

Item 113 is the gRNA of any one of the preceding Items, wherein the gRNA directs a nuclease to a target sequence for inducing a single-strand break within the target sequence.

Item 114 is the gRNA of any one of Items 111-113, wherein the nuclease is a Nine Cas9.

Item 115 is the gRNA of any one of the preceding Items, wherein the gRNA comprises a conservative substitution, optionally wherein the conservative substitution maintains at least one base pair.

Item 116 is a composition comprising a gRNA of any one of the preceding Items, associated with a lipid nanoparticle (LNP).

Item 117 is an LNP composition comprising a gRNA of any one of the preceding Items.

Item 118 is a composition comprising the gRNA of any one of Items 1-115, or the composition of any one of Items 116-117, further comprising a nuclease or an mRNA which encodes the nuclease.

Item 119 is the composition of Item 118, wherein the nuclease is a Cas protein.

Item 120 is the composition of Item 119, wherein the Cas protein is a Nme Cas9.

Item 121 is the composition of Item 120, wherein the Nine Cas9 is an Nme1 Cas9, an Nme2 Cas9, or an Nme3 Cas9.

Item 122 is the composition of any one of Items 118-121, wherein the nuclease has a double strand cleaving activity.

Item 123 is the composition of any one of Items 118-122, wherein the nuclease has a nickase activity.

Item 124 is the composition of any one of Items 118-123, wherein the nuclease has a dCas DNA binding domain.

Item 125 is the composition of any one of Items 118-124, wherein the nuclease is modified.

Item 126 is the composition of Item 125, wherein the modified nuclease comprises a heterologous functional domain.

Item 127 is the composition of Item 126 wherein the heterologous functional domain is a deaminase.

Item 128 is the composition of Item 127, further comprising a UGI or a mRNA encoding a UGI.

Item 129 is the composition of any one of Items 127-128, wherein the heterologous functional domain is a cytidine deaminase.

Item 130 is the composition of any one of Items 125-129, wherein the modified nuclease comprises a nuclear localization signal (NLS).

Item 131 is the composition of any one of Items 118-130, comprising an mRNA which encodes the nuclease.

Item 132 is the composition of Item 131, wherein the mRNA comprises the sequence of any one of SEQ ID NOs: 636-638.

Item 133 is a pharmaceutical formulation comprising the gRNA of any one of Items 1-115 or the composition of any one of Items 116-132 and a pharmaceutically acceptable carrier.

Item 134 is a method of modifying a target DNA comprising, delivering a Cas protein or a nucleic acid encoding a Cas protein, and any one or more of the following to a cell:
i. the gRNA of any one of Items 1-115;
ii. the composition of any one of Items 116-132; and
iii. the pharmaceutical formulation of Item 133.

Item 135 is the method of Item 134, wherein the method results in an insertion or deletion in a gene.

Item 136 is the method of Item 134 or 135, wherein the method results in at least one base edit.

Item 137 is the method of any one of Items 134-136, further comprising delivering to the cell a template, wherein at least a part of the template incorporates into a target DNA at or near a double strand break site induced by the Cas protein.

Item 138 is the gRNA of any one of Items 1-115, the composition of Items 116-132, or the pharmaceutical formulation of Item 133 for use in preparing a medicament for treating a disease or disorder.

Item 139 is use of the gRNA of any one of Items 1-115, the composition of Items 116-132, or the pharmaceutical formulation of Item 133 in the manufacture of a medicament for treating a disease or disorder.

We claim:

1. A guide RNA (gRNA) comprising a guide region and a conserved region, the conserved region comprising one or more of:
(a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 500; and
(ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
(i) one or more of nucleotides 82-86 and 91-95 is deleted and optionally one or more of positions 82-96 is substituted relative to SEQ ID NO: 500; and
(ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
(i) one or more of nucleotides 113-121 and 126-134 is deleted and optionally one or more of nucleotides 113-134 is substituted relative to SEQ ID NO: 500; and
(ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

2. The gRNA of claim 1, wherein the gRNA is a single-guide RNA (sgRNA) and wherein the gRNA comprises (a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 500; and
(ii) nucleotide 36 is linked to nucleotide 65 by at least 2 nucleotides.

3. The gRNA of claim 1 or 2, wherein the guide region has (i) an insertion of one nucleotide or a deletion of 1-4 nucleotides within positions 1-24 relative to SEQ ID NO: 500, or (ii) a length of 24 nucleotides.

4. The gRNA of claim 3, wherein the guide region has a length of 25, 24, 23, 22, 21, or 20 nucleotides, optionally wherein the guide region has a length of 25, 24, 23, or 22 nucleotides.

5. The gRNA of claim 4, wherein the guide region has a length of 23-24 nucleotides.

6. The gRNA of any one of claims 1-5, wherein the gRNA further comprises a 3′ tail.

7. The gRNA of claim 6, wherein the 3′ tail comprises 1, 2, 3, 4, 5, 6, 7, 8, 9, or 10 nucleotides.

8. The gRNA of claim 7, wherein the 3′ tail comprises 1, 2, 3, 4, or 5 nucleotides.

9. The gRNA of any one of claims 6-8, wherein the 3′ tail terminates with a nucleotide comprising a uracil or modified uracil.

10. The gRNA of any one of claims 6-9, wherein the 3′ tail is 1 nucleotide in length.

11. The gRNA of any one of claims 6-10, wherein the 3′ tail consists of a nucleotide comprising a uracil or a modified uracil.

12. The gRNA of any one of claims 6-11, wherein the 3′ tail comprises a modification of any one or more of the nucleotides present in the 3′ tail.

13. The gRNA of any one of claims 6-12, wherein the modification of the 3′ tail is one or more of 2′-O-methyl (2′-OMe) modified nucleotide and a phosphorothioate (PS) linkage between nucleotides.

14. The gRNA of any one of claims 6-13, wherein the 3′ tail is fully modified.

15. The gRNA of any one of claims 1-14, wherein the 3′ nucleotide of the gRNA is a nucleotide comprising a uracil or a modified uracil.

16. The gRNA of any one of claims 1-5, wherein one or more of nucleotides 144 and 145 are deleted relative to SEQ ID NO: 500.

17. The gRNA of any one of claims 1-5, wherein both nucleotides 144 and 145 are deleted relative to SEQ ID NO: 500.

18. The gRNA of any one of claims 1-5, wherein the gRNA does not comprise a 3′ tail.

19. The gRNA of any one of claims 1-18, wherein the shortened repeat/anti-repeat region lacks 2-24 nucleotides.

20. The gRNA of any one of claims 1-19, wherein the shortened repeat/anti-repeat region has a length of 28, 29, 30, 31, 32, 33, 34, 35, 36, 37, 38, 39, 40, 41, 42, 43, 44, 45, 46, 47, 48, 49, or 50 nucleotides.

21. The gRNA of any one of claims 1-20, wherein the shortened repeat/anti-repeat region lacks 12-24, optionally 18-24 nucleotides, optionally 20-22 nucleotides.

22. The gRNA of any one of claims 1-21, wherein the shortened repeat/anti-repeat region has a length of 28, 29, 30, 31, 32, 33, 34, 35, 36, 37, 38, 39, or 40 nucleotides.

23. The gRNA of any one of claims 1-22, wherein the shortened repeat/anti-repeat region has a length of 28, 29, 30, 31, 32, 33, or 34 nucleotides, or 30, 31, or 32 nucleotides.

24. The gRNA of any one of claims 1-23, wherein nucleotides 37-64 of SEQ ID NO: 500 form the upper stem, and one or more base pairs of the upper stem of the shortened repeat/anti-repeat region are deleted.

25. The gRNA of any one of claims 1-24, wherein the upper stem of the shortened repeat/anti-repeat region comprises no more than one, two, three, or four base pairs.

26. The gRNA of any one of claims 1-25, wherein all of positions 39-48 and all of positions 53-62 of the upper stem of the shortened repeat/anti-repeat region are deleted, and optionally nucleotide 38 or 63 is substituted.

27. The gRNA of any one of claims 1-26, wherein all of positions 38-48 and all of positions 53-63 of the upper stem of the shortened repeat/anti-repeat region are deleted, and optionally nucleotide 37 or 64 is substituted.

28. The gRNA of any one of claims 1-27, wherein all of nucleotides 37-48 and 53-64 of the upper stem of the shortened repeat/anti-repeat region are deleted, and optionally nucleotides 36 or 65 is substituted.

29. The Grna of any one of claims 1-28, wherein the shortened repeat/anti-repeat region has a duplex portion 11 base paired nucleotides in length.

30. The gRNA of any one of claims 1-29, wherein the shortened repeat/anti-repeat region has a single duplex portion.

31. The gRNA of any one of claims 1-29, wherein the shortened repeat/anti-repeat region has a first duplex portion and a second duplex portion.

32. The gRNA of claim 31, wherein the second duplex portion is 2-3 base paired nucleotides in length.

33. The gRNA of claim 31, wherein the first duplex portion is 11 base paired nucleotides in length and the second duplex portion is 3 base paired nucleotides in length.

34. The gRNA of any one of claims 1-33, wherein the upper stem of the shortened repeat/anti-repeat region includes one or more substitutions relative to SEQ ID NO: 500.

35. The gRNA of any one of claims 1-34, wherein one or more of nucleotides 49-52 is substituted relative to SEQ ID NO: 500.

36. The gRNA of any one of claims 1-33, wherein the shortened repeat/anti-repeat region is unsubstituted.

37. The gRNA of any one of claims 1-36, wherein the shortened repeat/anti-repeat region has 12-22 modified nucleotides

38. The gRNA of claim 37, wherein the shortened repeat/anti-repeat region does not comprise a modification at nucleotide 76.

39. The gRNA of claim 37, wherein the shortened repeat/anti-repeat does not comprise a phosphorothioate (PS) modification at nucleotide 76.

40. The gRNA of any one of claims 1-39, wherein the shortened hairpin 1 region lacks 2-10 nucleotides, optionally 2-8 or 2-4 nucleotides.

41. The gRNA of any one of claims 1-40, wherein the shortened hairpin 1 region has a length of 13, 14, 15, 16, 17, 18, 19, 20, or 21 nucleotides.

42. The gRNA of claim any one of claims 1-41, wherein the shortened hairpin 1 region has a duplex portion 4-8, optionally 7-8 base paired nucleotides in length.

43. The gRNA of claim any one of claims 1-41, wherein the shortened hairpin 1 region has a single duplex portion.

44. The gRNA of any one of claims 1-43, wherein one or two base pairs of the shortened hairpin 1 region are deleted.

45. The gRNA of any one of claims 1-44, wherein the stem of the shortened hairpin 1 region is seven or eight base paired nucleotides in length.

46. The gRNA of any one of claims 1-45, wherein one or more of positions 85-86 and one or more of nucleotides 91-92 of the shortened hairpin 1 region are deleted.

47. The gRNA of any one of claims 1-46, wherein nucleotides 86 and 91 or nucleotides 85 and 92 of the shortened hairpin 1 region are deleted.

48. The gRNA of any one of claims 1-47, wherein one or more of nucleotides 82-95 of the shortened hairpin 1 region is substituted relative to SEQ ID NO: 500.

49. The gRNA of any one of claims 1-48, wherein one or more of nucleotides 87-90 is substituted relative to SEQ ID NO: 500.

50. The gRNA of any one of claims 1-48, wherein the shortened hairpin 1 region is unsubstituted.

51. The gRNA of any one of claims 1-49, wherein the shortened hairpin 1 region has 6-15 modified nucleotides.

52. The gRNA of any one of claims 1-50, wherein the shortened hairpin 2 region lacks 2-18, optionally 2-16 nucleotides.

53. The gRNA of any one of claims 1-51, wherein the shortened hairpin 2 region has a length of 24, 25, 26, 27, 28, 29, 30, 31, 32, 33, 34, 35, 36, 37, 38, 39, or 40 nucleotides.

54. The gRNA of any one of claims 1-52, wherein the shortened hairpin 2 region has a length of 28, 29, 30, 31, 32, 33, or 34 nucleotides.

55. The gRNA of any one of claims 1-53, wherein one or more of nucleotides 113-121 and one or more of nucleotides 126-134 of the shortened hairpin 2 region are deleted.

56. The gRNA of any one of claims 1-54, wherein the shortened hairpin 2 region comprises an unpaired region.

57. The gRNA of any one of claims 1-55, wherein the shortened hairpin 2 region has two duplex portions.

58. The gRNA of any one of claims 1-56, wherein the shortened hairpin 2 region has a duplex portion of 4 base paired nucleotides in length.

59. The gRNA of any one of claims 57-58, wherein the shortened hairpin 2 region has a duplex portion of 4-8 base paired nucleotides in length.

60. The gRNA of any one of claims 57-59, wherein the shortened hairpin 2 region has a duplex portion of 4-6 base paired nucleotides in length.

61. The gRNA of any one of claims 1-60, wherein nucleotides 109-138 of SEQ ID NO: 500 form the upper stem, and the upper stem of the shortened hairpin 2 region comprises one, two, three, or four base pairs.

62. The gRNA of any one of claims 1-61, wherein at least one pair of nucleotides 113 and 134, nucleotides 114 and 133, nucleotides 115 and 132, nucleotides 116 and 131, nucleotides 117 and 130, nucleotides 118 and 129, nucleotides 119 and 128, nucleotides 120 and 127, and nucleotides 121 and 126 are deleted.

63. The gRNA of any one of claims 1-62, wherein all of positions 113-121 and 126-134 of the shortened hairpin 2 region are deleted.

64. The gRNA of any one of claims 1-63, wherein one or more of nucleotides 113-134 of the shortened hairpin 2 region is substituted relative to SEQ ID NO: 500.

65. The gRNA of any one of claims 1-64, wherein one or more of nucleotides 122-125 is substituted relative to SEQ ID NO: 500.

66. The gRNA of any one of claims 1-64, wherein the shortened hairpin 2 region is unsubstituted.

67. The gRNA of claim any one of claims 1-66, wherein the shortened hairpin 2 region has 6-15 modified nucleotides.

68. The gRNA of any one of claims 1-67, wherein the guide region of the gRNA comprises at least two modified nucleotides, optionally at least four modified nucleotides.

69. The gRNA of any one of claims 1-68, wherein the guide region of the gRNA comprises 1, 2, 3, 4, 5, 6, 7, 8, 9, 10, 11, or 12 modified nucleotides, optionally 1, 2, or 3 modified nucleotides.

70. The gRNA of any one of claims 1-69, wherein the guide region of the gRNA comprises 4, 5, 6, 7, 8, 9, 10, 11, or 12 modified nucleotides.

71. The gRNA of any one of claims 1-70, wherein the guide region of the gRNA comprises 6, 7, 8, 9, 10, 11, or 12 modified nucleotides.

72. The gRNA of any one of claims 1-71, wherein the guide region does not comprise a modified nucleotide 3′ of the first three nucleotides of the guide region.

73. The gRNA of any one of claims 1-66, wherein the guide region does not comprise a modified nucleotide.

74. The gRNA of any one of claims 1-72, wherein the gRNA comprises a 5′ end modification.

75. The gRNA of any one of claims 1-74, wherein the gRNA comprises a 3′ end modification.

76. The gRNA of any one of claims 1-75, wherein the gRNA comprises a 5′ end modification and a 3′ end modification.

77. The gRNA of any one of claims 1-76, comprising a modification in the upper stem region of the repeat/anti-repeat region.

78. The gRNA of any one of claims 1-77, comprising a modification in the hairpin 1 region.

79. The gRNA of any one of claims 1-78, comprising a modification in the hairpin 2 region.

80. The gRNA of claim 79, wherein the modification in the hairpin 2 region comprises a modification at 1, 2, 3, or 4 nucleotides of nucleotides 106-109.

81. The gRNA of claim 80, wherein the modification in the hairpin 2 region comprises a modification at each of nucleotides 106-109.

82. The gRNA of any one of claim 80 or 81, wherein the modification comprises a 2′-O-methyl (2′-O-Me) modification.

83. The gRNA of any one of claims 1-82, comprising a 3′ end modification, and comprising a modification in the upper stem region of the repeat/anti-repeat region.

84. The gRNA of any one of claims 1-83, comprising a 3′ end modification, and a modification in the hairpin 1 region.

85. The gRNA of any one of claims 1-83, comprising a 3′ end modification, and a modification in the hairpin 2 region.

86. The gRNA of any one of claims 1-85, comprising a 5′ end modification, and comprising a modification in the upper stem region of the repeat/anti-repeat region.

87. The gRNA of any one of claims 1-86, comprising a 5′ end modification, and a modification in the hairpin 1 region.

88. The gRNA of any one of claims 1-87, comprising a 5′ end modification, and a modification in the hairpin 2 region.

89. The gRNA of any one of claims 1-88, comprising a 5′ end modification, a modification in the upper stem region of the repeat/anti-repeat region, and a 3′ end modification.

90. The gRNA of any one of claims 1-89, comprising a 5′ end modification, a modification in the hairpin 1 region, and a 3′ end modification.

91. The gRNA of any one of claims 1-90, comprising a 5′ end modification, a modification in the hairpin 1 region, a modification in the hairpin 2 region, and a 3′ end modification.

92. The gRNA of any one of claims 1-91, comprising a 5′ end modification, a modification in the repeat/anti-repeat region, a modification in the hairpin 1 region, a modification in the hairpin 2 region, and a 3′ end modification.

93. The gRNA of any one of claims 1-92, wherein the modification in the repeat/anti-repeat region does not comprise a phosphorothioate (PS) modification at nucleotide 76.

94. The gRNA of any one of claims 1-93, wherein the modification in the repeat/anti-repeat region does not comprise a modification at nucleotide 76.

95. The gRNA of any one of claims 74-94, wherein the 5′ end modification comprises a modified nucleotide selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

96. The gRNA of any one of the claims 74-95, wherein the 3′ end modification comprises a modified nucleotide selected from a 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide.

97. The gRNA of any one of the claims 74-96, wherein the 5′ end modification comprises any of:
i. a modification of any one or more of the first 1, 2, 3, or 4 nucleotides;
ii. one modified nucleotide;
iii. two modified nucleotides;
iv. three modified nucleotides; and
v. four modified nucleotides.

98. The gRNA of any one of claims 74-97, wherein the 5′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide; and
v. an inverted abasic modified nucleotide.

99. The gRNA of any one of claims 74-98, wherein the 3′ end modification comprises any of:
i. a modification of any one or more of the last 4, 3, 2, or 1 nucleotides;
ii. one modified nucleotide;
iii. two modified nucleotides;
iv. three modified nucleotides; and
v. four modified nucleotides.

100. The gRNA of any one of claims 74-99, wherein the 3′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide; and
v. an inverted abasic modified nucleotide.

101. The gRNA of any one of claims 74-100, wherein the 5′ end modification comprises at least one PS linkage, and wherein one or more of:
i. there is one PS linkage, and the linkage is between the first and second nucleotides;
ii. there are two PS linkages between the first three nucleotides;
iii. there are PS linkages between any one or more of the first four nucleotides; and
iv. there are PS linkages between any one or more of the first five nucleotides.

102. The gRNA of claim 101, wherein the 5′ end modification further comprises at least one 2′-OMe, 2′-O-moe, inverted abasic, or 2′-F modified nucleotide.

103. The gRNA of any one of claims 1-102, wherein the 5′ end modification comprises:
i. a modification of one or more of the first 1-4 nucleotides, wherein the modification is a PS linkage, inverted abasic nucleotide, 2′-OMe, 2′-O-moe, or 2′-F;
ii. a modification to the first nucleotide with 2′-Ome, 2′-O-moe, or 2′-F, and an optional one or two PS linkages to the next nucleotide or the first nucleotide of the 3′ tail;
iii. a modification to the first or second nucleotide with 2′-Ome, 2′-O-moe, or 2′-F, and optionally one or more PS linkages;
iv. a modification to the first, second, or third nucleotides with 2′-Ome, 2′-O-moe, or 2′-F, and optionally one or more PS linkages; or
v. a modification to the first, second, third, or forth nucleotides with 2′-Ome, 2′-O-moe, or 2′-F, and optionally one or more PS linkages.

104. The gRNA of any one of claims 1-103, wherein the 3′ end modification comprises at least one PS linkage, and wherein one or more of:
vi. there is one PS linkage, and the linkage is between the last and second to last nucleotides;
vii. there are two PS linkages between the last three nucleotides; and
viii. there are PS linkages between any one or more of the last four nucleotides.

105. The gRNA of claim 104, wherein the 3′ end modification further comprises at least one 2′-Ome, 2′-O-moe, inverted abasic, or 2′-F modified nucleotide.

106. The gRNA of any one of claims 1-105, wherein the 3′ end modification comprises:
ix. a modification of one or more of the last 1-4 nucleotides, wherein the modification is a PS linkage, inverted abasic nucleotide, 2′-OMe, 2′-O-moe, or 2′-F;
x. a modification to the last nucleotide with 2′-OMe, 2′-O-moe, or 2′-F, and an optional one or two PS linkages to the next nucleotide or the first nucleotide of the 3′ tail;
xi. a modification to the last or second to last nucleotide with 2′-OMe, 2′-O-moe, or 2′-F, and optionally one or more PS linkages;
xii. a modification to the last, second to last, or third to last nucleotides with 2′-OMe, 2′-O-moe, or 2′-F, and optionally one or more PS linkages; or
xiii. a modification to the last, second to last, third to last, or fourth to last nucleotides with 2′-OMe, 2′-O-moe, or 2′-F, and optionally one or more PS linkages.

107. The gRNA of any one of claims 1-106, wherein the modification in the repeat/anti-repeat region, the hairpin 1 region, or the hairpin 2 region comprises a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, or a phosphorothioate (PS) linkage between nucleotides.

108. The gRNA of any one of claims 1-106, wherein the modification in the repeat/anti-repeat region, the hairpin 1 region, or the hairpin 2 region comprises a modified nucleotide selected from 2′-O-methyl (2′-OMe) modified nucleotide, a 2′-fluoro (2′-F) modified nucleotide, or a phosphorothioate (PS) linkage between nucleotides.

109. The gRNA of any one of claims 1-106, wherein the modification in the repeat/anti-repeat region, the hairpin 1 region, or the hairpin 2 region comprises a modified nucleotide selected from 2′-O-methyl (2′-OMe) modified nucleotide or a phosphorothioate (PS) linkage between nucleotides.

110. The gRNA of any one of claims 1-109, wherein the modification in the repeat/anti-repeat region does not comprise a phosphorothioate modification at nucleotide 76.

111. The gRNA of any one of claims 1-110, wherein the modification in the repeat/anti-repeat region does not comprise a modification at nucleotide 76.

112. The gRNA of any one of claims 1-111, wherein at least 20%, 30%, 40%, or 50% of the nucleotides are modified nucleotides.

113. The gRNA of claim 112, wherein the gRNA comprises modified nucleotides selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, or combinations thereof.

114. The gRNA of any one of claims 1-113, wherein the modification comprises a modification at 1, 2, 3, or 4 nucleotides of nucleotides 106-109.

115. The gRNA of any one of claim 113 or 114, wherein the modification comprises a modification at each of nucleotides 106-109.

116. The gRNA of any one of claims 114-115, wherein the modification comprises a 2′-O-methyl modification.

117. The gRNA of any one of claims 112-116, wherein the gRNA comprises modified nucleotides selected from 2′-O-methyl (2′-Ome) modified nucleotide, a 2′-fluoro (2′-F) modified nucleotide, a phosphorothioate (PS) linkage between nucleotides, or combinations thereof.

118. The gRNA of any one of claims 1-117, wherein nucleotides 1-3 of the guide region are modified and nucleotides in the guide region other than nucleotides 1-3 are not modified.

119. The gRNA of any one of claims 1-118, wherein a 3′ tail of nucleotide 144 is present in the gRNA, and comprises 2′-O-Me modified nucleotides at nucleotides 141-144 and two PS linkages between nucleotides 141-142 and 142-143 respectively.

120. The gRNA of any one of claims 1-120, wherein one or more positions of 49-52, 87-90, or 122-125 is substituted.

121. A single guide RNA (sgRNA) comprising any one of SEQ ID NOs: 1-19 and 21-42.

122. The gRNA of any one of claims 1-121, comprising a nucleotide sequence having at least 99, 98, 97, 96, 95, 94, 93, 92, 91, 90, 85, 80, 75, or 70% identity to the nucleotide sequence of any one of SEQ ID Nos: 1-19 and 21-42.

123. The gRNA of any one of claims 1-121, comprising a nucleotide sequence having at least 99, 98, 97, 96, 95, 94, 93, 92, 91, 90, 85, 80, 75, or 70% identity to the nucleotide sequence of any one of SEQ ID Nos: 1-19 and 21-42, wherein the modification at each nucleotide of the gRNA that corresponds to a nucleotide of the reference sequence identifier in Table 1 is identical to or equivalent to the modification shown in the reference sequence identifier in Table 2.

124. The gRNA of any one of claims 1-122, comprising a nucleotide sequence having at least 99, 98, 97, 96, 95, 94, 93, 92, 91, or 90% identity to the sequence from X to the 3′ end of the nucleotide sequence of any one of SEQ ID Nos: 1-5, 7, 8, 11, 12, 13, 15, 16, 18, 19, 21, 23, 24, 26, 27, 28, 30, 31, 33, 34, 35, 37, 39, 41, 101-291, 301-494, 931-946, 951, and 952, where X is the first nucleotide of the conserved region.

125. The gRNA of any one of claims 121-124, further comprising a 3′ tail comprising a 2′-O-Me modified nucleotide.

126. The gRNA of any one of claims 1-125, wherein the gRNA directs a nuclease to a target sequence for binding.

127. The gRNA of any one of claims 1-126, wherein the gRNA directs a nuclease to a target sequence for inducing a double-strand break within the target sequence.

128. The gRNA of any one of claims 1-127, wherein the gRNA directs a nuclease to a target sequence for inducing a single-strand break within the target sequence.

129. The gRNA of any one of claims 126-129, wherein the nuclease is a Nme Cas9.

130. The gRNA of any one of claims 1-129, wherein the gRNA comprises a conservative substitution, optionally wherein the conservative substitution maintains at least one base pair.

131. A composition comprising a gRNA of any one of claims 1-130, associated with a lipid nanoparticle (LNP).

132. An LNP composition comprising a gRNA of any one of claims 1-130.

133. A composition comprising the gRNA of any one of claims 1-130, or the composition of any one of claims 131-132, further comprising a nuclease or an mRNA which encodes the nuclease.

134. The composition of claim 133, wherein the nuclease is a Cas protein.

135. The composition of claim 134, wherein the Cas protein is a Nine Cas9.

136. The composition of claim 135, wherein the Nine Cas9 is an Nme1 Cas9, an Nme2 Cas9, or an Nme3 Cas9.

137. The composition of any one of claims 133-136, wherein the nuclease has a double strand cleaving activity.

138. The composition of any one of claims 133-137, wherein the nuclease has a nickase activity.

139. The composition of any one of claims 133-138, wherein the nuclease has a dCas DNA binding domain.

140. The composition of any one of claims 133-139, wherein the nuclease is modified.

141. The composition of claim 140, wherein the modified nuclease comprises a heterologous functional domain.

142. The composition of claim 141, wherein the heterologous functional domain is a deaminase.

143. The composition of claim 142, further comprising a UGI or a mRNA encoding a UGI.

144. The composition of any one of claims 142-143, wherein the heterologous functional domain is a cytidine deaminase.

145. The composition of any one of claims 140-144, wherein the modified nuclease comprises a nuclear localization signal (NLS).

146. The composition of any one of claims 133-145, comprising an mRNA which encodes the nuclease.

147. The composition of claim 146, wherein the mRNA comprises the sequence of any one of SEQ ID NOs: 636-638.

148. A pharmaceutical formulation comprising the gRNA of any one of claims 1-130 or the composition of any one of claims 131-147 and a pharmaceutically acceptable carrier.

149. A method of modifying a target DNA comprising, delivering a Cas protein or a nucleic acid encoding a Cas protein, and any one or more of the following to a cell:
i. the gRNA of any one of claims 1-130;
ii. the composition of any one of claims 131-147; and
iii. the pharmaceutical formulation of claim 148.

150. The method of claim 149, wherein the method results in an insertion or deletion in a gene.

151. The method of claim 149 or 150, wherein the method results in at least one base edit.

152. The method of any one of claims 149-151, further comprising delivering to the cell a template, wherein at least a part of the template incorporates into a target DNA at or near a double strand break site induced by the Cas protein.

153. The gRNA of any one of claims 1-130, the composition of claims 131-147, or the pharmaceutical formulation of claim 148 for use in preparing a medicament for treating a disease or disorder.

154. Use of the gRNA of any one of claims 1-130, the composition of claims 131-147, or the pharmaceutical formulation of claim 148 in the manufacture of a medicament for treating a disease or disorder.
